SEQUENCE LISTING (134 sequences)

SEQ ID NO: 1 - protein, 488 aa, Artificial Sequence
  gp120 variant ST-008
GAAEQLWVTVYFGVPVWEEATTTLFCASDAKAYDTEVHNIWATHACVPTDPNPQEVVLENVTENFNMWKNNMVDQMHEDIISLWDQSLKPCIKLIPLCVTLDCIDVGNATNGGDTTNTTSSSGVRMEPGEIKNCSFNITTSIRDKMQKEYALLYKLDLVPIDNGNSNGSNTTNYRLISCNTSVITQACPKVTFEPIPIHYCAPAGFAILKCNNKTFNGTGPCRNVSTVQCTHGIRPVVSTQLLLNGSLAEEEVVIRSENFSDNAKTIIVQLNESVKINCTRPNNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKR

SEQ ID NO: 2 - protein, 478 aa, Artificial Sequence
  gp120 variant ST-051
SAVEKLWVTVYYGVPVWKEATTTLFCASDAKAYDAEAHNVWATHACVPTDPNPQEVVLENVTENFNMWKNNMVEQMHEDIISLWDQSLKPCVKLTPLCVTLNCTNYVKIDTNATVYNWEKMEEGEIKNCSFNVTTAIRDKVQKEYALFYKLDVVPIEENGNTSYRLINCNTSIITQACPKVSFEPIPIHYCAPAGFAILKCNNKTFNGSGPCKNVSTVQCTHGIRPVVSTQLLLNGSLAEEEVILRSENFTSNAKTIIVQLNETIKINCTRPNNNTRKGIHIGPGRAFYTTGQIIGDIRKAHCNLSRAQWNNTLKQIVEKLREQFNNKTIVFTHSSGGDPEIVMHSFNCGGEFFYCNSTQLFNSTWNDTEKSSGTEGNDTIILPCRIKQIVNMWQEVGKAMYAPPIRGKIYCRSNITGLLLTRDGGKNESEIEIFRPGGGDMRDNWRSELYKYKVVKIEPLGIAPTKAKGRVVQREKR

SEQ ID NO: 3 - protein, 479 aa, Artificial Sequence
  gp120 variant ST-080
SATERFWVTVYYGVPVWKETTTTLFCASDAKAYDTEVHNVWATHACVPTDPNPQEVVLENVTENFNMWDNNMVDQMNEDIISLWDQSLKPCVTITPLCVTLHCTNYVKIDTNATVYNWEKMEEGEIKNCSFNVTTAIRDKVQKEYALFYKLDVVPIEENGNTSYRLINCNTSVITQACPKVSFEPIPIHYCAPAGFAILKCNDKKFNGKGPCTNVSTVQCTHGIRPVVSTQLLLNGSLAEEKVVIRSDNFTDNAKTIIVQLNESVKINCTRPSNNTRKSIHIGPGRASYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGNSTETETEIFRPGGGDMRDNWRSELYKYKVVKIEPLGVAPNKAKRRVVQREKR

SEQ ID NO: 4 - protein, 482 aa, Artificial Sequence
  gp120 variant ST-140
SAEENLWVTVYYGVPVWKEADTTLFCASDAKAYDREVHNVWATHACVPTDPNPQEVVLGNVTENFNMWKNSMVDQMREDIISLWDQSLKPCVKLTPLCVTLNCTNYVKIDTNATVYNWEKMEEGEIKNCSFNVTTAIRDKVQKEYALFYKLDVVPIEENGNTSYRLINCNTSIITQACPKVSFEPIPIHYCAPAGFAILKCNNKTFNGTGPCTNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIVNMWQEVGKAMYAPPISGQIRCSSNITGLLLTRDGGENNGTENNETETFGPGGGDMRDNWRSELYKYKVVKIEPLGVAPTRAKRRVVQREKR

SEQ ID NO: 5 - protein, 494 aa, Artificial Sequence
  gp120 variant ST-148
SVAEKMWVTVYYGVPVWKEATTTLFCASDAKAYDTEVHNVWATHACVPTDPNPQEVVLKNVTENFNMWKSNMVEQMHEDIISLWDQSLKPCVKLTPLCVTLNCTNLNITRNTTNPTSSSWGMMEKGEIKNCSFNVTTAIRDKVQKEYALFYKLDVVPIEENGNTSYRLINCNTSTITQACPKVSFEPIPIHYCAPAGFAILKCNNKTFNGTGPCTNVSTVQCTHGIRPAVSTQLLLNGSLAEEEVVIRSENFSDNAKTIIVQLNQSVEINCTRPNNNTRRSINIGPGRALYATGEIIGDIRQAHCNISETQWNNTLKQIVKKLREQFKNKTIAFNQSSGGDPEIVMHSFNCGGEFFYCNTTQLFNSTWYRNGTWYWKGTRIENGTEGLNDTIITLPCRIKQIVNMWQEVGKAMYAPPIKGQIRCSSNITGLLLTRDGGENNGTENNETETFRPGGGNMRDNWRSELYKYKVVKIEPIGVAPTRAKRRTVQREKR

SEQ ID NO: 6 - protein, 486 aa, Artificial Sequence
  gp120 variant ST-161
SAVEQLWVTVYYGVPVWKEATTTLFCASDAKAYKTEVHNVWATHACVPTDPNPQEVVLKNVTENFNMWDNNMVDQMNEDIISLWDQSLKPCVKLTPLCVTLNCTNVKKGTNATNSSEEMRERGEIKNCSFHVTSSIKDKTTTEHALFYKLDVVPIDARNNSENGSYILISCNTSTITQACPKVSFEPIPIHYCAPAGFAILKCNDKTFNGSGPCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGLNDTIITLPCRIKQIVNMWQEVGKAMYAPPISGQIRCSSNITGLLLTRDGGENNGTENNETETFRPGGGDMRDNWRSELYKYKVVKIEPLGVAPTKAKRRVVQREKR

SEQ ID NO: 7 - protein, 479 aa, Artificial Sequence
  gp120 variant ST-188
SVAEKMWVTVYYGVPVWREATTTLFCVSDAKAYDTEVHNVWATHACVPTDPNPQEVVLKNVTENFNMWKNNMVDQMHEDIISLWDESLKPCVKLTPLCVTLNCTNYVKIDTNATVYNWEKMEEGEIKNCSFNVTTAIRDKVQKEYALFYKLDVVPIEENGNTSYRLINCNTSIITQACPKVTFEPIPIHYCAPAGFAILKCNNKTFNGTGPCTNVSTVQCTHGIRPVVSTQLLLNGSLAEEEVVIRSENFTNNAKTIIIQLKESVVINCTRPNNNTRKRISIGPGRVWYTTGQIIGDIRKAHCNLSSTQWNNTLGQIVEKLRAQFGNKTIRFNQPSGGDPEIVLHSFNCGGEFFYCNTSKLFNSTWNDTKRSNIEGTITLPCRIKQFINMWQTVGKAMYAPPIKGQIRCSSKISGLLLTRDGGENNGTENNEIETFRPGGGDMRDNWRSELYKYKVVRIEPIGVAPTRAKRRTVQREKR

SEQ ID NO: 8 - protein, 313 aa, Artificial Sequence
  gp120 core variant L7-010
EVVLENVTENFNMWKNNMVEQMHEDIISLWDQSLKPCVKLTPLCVGAGSCNTSTITQACPKVSFEPIPIHYCAPAGFAILKCNDKKFNGSGPCKNVSTVQCTHGIKPVVSTQLLLNGSLAEEDIVIRSENFTDNAKTIIVQLNESVVINCTGAGHCNLSSTQWNNTLGQIVEKLRAQFGNKTIRFNQPSGGDPEIVLHSFNCGGEFFYCNTSQLFNSTWNDTKRSNIDGTITLPCRIKQIVNMWQEVGKAMYAPPIRGQIRCSSNITGLLLTRDGGENNGTENNETETFRPGGGGMRDNWRSELYKYKVVKIE

SEQ ID NO: 9 - protein, 313 aa, Artificial Sequence
  gp120 core variant L7-028
EVVLENVTENFNMWKNNMVEQMHEDIISLWDQSLKPCVKLTPLCVGAGSCNTSTITQACPKVSFEPIPIHYCAPAGFAILKCNDKKFNGTGPCRNVSTVQCTHGIRPVVSTQLLLNGSPAEEEVVIRSENFTDNAKTIIVQLNESVVINCTGAGHCNISSTQWNNTLGQIVGKLRAQFGNKTIRFNQPSGGDPEIVLHSFNCGGEFFYCNTSKLFNSTWNDTKRPNIDGTITLPCRIKQIVNMWQGVGKAMYAPPIRGQISCSSNITGLLLTRDGGENNGTENNETETFRPGGGDMRDYWRSELYKYKVVKIE

SEQ ID NO: 10 - protein, 312 aa, Artificial Sequence
  gp120 core variant L7-043
EVVLENVAENFNMWKNNMVEQMHEDIIGLWDQSLKPCVKLTPLCVGAGSCNTSTITQARPKVSFEPIPIHYCAPAGFAILKCNDKKFNGTGPCKNVSTVQCTHGIRPVVSTQLLLNGSLAEEEVVIRSENFSDNAKTIIVQLNESVVINCTGAGHCNISSTQWNNTLGQIVEKLRAQFGNKTIRFNQPSGGDPEIVLHSFNCGGEFFYCNTSKLFNSTWNDTKRSNIDGTITLPCRIKQIVNMWQEVGKAMYAPPIKGQIRCSSNITGLLLTRDGGENNGTENNETETFRPGGDMRDNWRSELYKYKVVKIE

SEQ ID NO: 11 - protein, 318 aa, Artificial Sequence
  gp120 core variant L7-068
EVVLENVTENFNMWDNNMVEQMHEDVINLWDQSLKPCVKLTPLCVGAGSCNTSTITQACPKVSFEPIPIHYCAPAGFAILKCNDKKFNGTGPCTNVSTVQCTHGIRPVVSTQLLLNGGLAEEEVVIRSENFTNNAKTIIIQLKESVVINCTGAGHCNLSSTQWNNTLGQIVEKLRARFGNKTIRFNQPSGGDPEIVLHSFNCGGEFFYCNTSKLFNSTWNGTEGLDETNNGTGGNITLPCRIKQIVNMWQEVGKAMYAPPIRGQIRCSSNITGLLLTRDGGENNGTENNETETFRPGGGDMRDYWRSELYKYKVVKIE

SEQ ID NO: 12 - protein, 320 aa, Artificial Sequence
  gp120 core variant L7-084
EVVLENVTENFNMWKNNMVEQMREDVINLWDQSLKPCVKLTPLCVGAGSCDTSVITQACPKVTFEPIPIHHCAPAGFATLKCNDKKFNGTGPCRNVSTVQCTHGIKPVVSTQLLLNGSLAEGDIVIRSENFTDNAKTIIVQLRESVVINCTGAGHCNISSTQWNNTLGQIVEKLRAQFGNKTIRFNQPSGGDPEIVLHSFNCGGEFFYCNTSKLFNSTWNSTWGDTEMNNTEESNNNITLPCRIKQIVNMWQEVGKAMYAPPVRGQISCSSNITGLLLTRDGGENNGTENNETETFRPGGGGMRDNWRSELYKYKVVKIE

SEQ ID NO: 13 - protein, 315 aa, Artificial Sequence
  gp120 core variant L7-098
EVVLENVTENFNMWKNNMVEQMQEDIIGLWDQSLKPRVKLTPLCVGAGSCNTSVITQACPKVTFEPIPIHYCAPAGFAILKCNDKKFNGSGPCKNVSTVQCTHGIRPVVSTQLLLNGSLAEEEVVIRSENFTNNAKTIIVQLNESVKINCTGAGHCNLSSTQWNNTLGQIVGKLRAQFGNKTIRFNQPSGGDPEIVLHSFNCGGEFFYCNTSKLFNSTWNDTKRSNIEGNDTIILPCRIKRIINTWQEVGKAMYTPPVRGQISCSSNITGLLLTRDGGENNGTENNETETFRPGGGDMGDNWRSELYKYKVVKIE

SEQ ID NO: 14 - protein, 313 aa, Artificial Sequence
  gp120 core variant L7-105
EVVLENVTENFNMWKNNMVEQMHEDIIGLWDQSLKPCVKLTPLCVGAGSCNTSTITQACPKVSFEPIPIHYCAPAGFAILKCNDKKFNGTGPCKNVSTVQCTHGIRPVVSTQLLLNGSLAEEDIVIRSENFTDNAKTIIVQLNESVVINCTGAGHCNISSTQWNNTLGQIVGKLRAQFGNKTIRFNQPSGGDPEIVLHSFNCGGEFFYCNTSKLFNSTWNDTKRSNIDGTITLPCRIKQIVNMWQEVGKAMYAPPIRGQIRCSSNITGLLLTRDGGENNGTENNETETFRPEGGDMRDYWRSELYKYKVVKIE

SEQ ID NO: 15 - protein, 345 aa, Artificial Sequence
  gp120 core variant L7-010CDC
EVVLENVTENFNMWKNNMVEQMHEDIISLWDQSLKPCVKLTPLCVGAGSCNTSTITQACPKVSFEPIPIHYCAPAGFAILKCNDKKFNGSGPCKNVSTVQCTHGIKPVVSTQLLLNGSLAEEDIVIRSENFTDNAKTIIVQLNESVVINCTGAGHCNLSSTQWNNTLGQIVEKLRAQFGNKTIRFNQPSGGDPEIVLHSFNCGGEFFYCNTSQLFNSTWNDTKRSNIDGTITLPCRIKQIVNMWQEVGKAMYAPPIRGQIRCSSNITGLLLTRDGGENNGTENNETETFRPGGGGMRDNWRSELYKYKVVKIEAGNRGPPTLDDQPRLCLLVASHLLFAPPPCLP

SEQ ID NO: 16 - protein, 345 aa, Artificial Sequence
  gp120 core variant L7-028CDC
EVVLENVTENFNMWKNNMVEQMHEDIISLWDQSLKPCVKLTPLCVGAGSCNTSTITQACPKVSFEPIPIHYCAPAGFAILKCNDKKFNGTGPCRNVSTVQCTHGIRPVVSTQLLLNGSPAEEEVVIRSENFTDNAKTIIVQLNESVVINCTGAGHCNISSTQWNNTLGQIVGKLRAQFGNKTIRFNQPSGGDPEIVLHSFNCGGEFFYCNTSKLFNSTWNDTKRPNIDGTITLPCRIKQIVNMWQGVGKAMYAPPIRGQISCSSNITGLLLTRDGGENNGTENNETETFRPGGGDMRDYWRSELYKYKVVKIEAGNRGPPTLDDQPRLCLLVASHLLFAPPPCLP

SEQ ID NO: 17 - protein, 344 aa, Artificial Sequence
  gp120 core variant L7-043CDC
EVVLENVAENFNMWKNNMVEQMHEDIIGLWDQSLKPCVKLTPLCVGAGSCNTSTITQARPKVSFEPIPIHYCAPAGFAILKCNDKKFNGTGPCKNVSTVQCTHGIRPVVSTQLLLNGSLAEEEVVIRSENFSDNAKTIIVQLNESVVINCTGAGHCNISSTQWNNTLGQIVEKLRAQFGNKTIRFNQPSGGDPEIVLHSFNCGGEFFYCNTSKLFNSTWNDTKRSNIDGTITLPCRIKQIVNMWQEVGKAMYAPPIKGQIRCSSNITGLLLTRDGGENNGTENNETETFRPGGDMRDNWRSELYKYKVVKIEAGNRGPPTLDDQPRLCLLVASHLLFAPPPCLP

SEQ ID NO: 18 - protein, 350 aa, Artificial Sequence
  gp120 core variant L7-068CDC
EVVLENVTENFNMWDNNMVEQMHEDVINLWDQSLKPCVKLTPLCVGAGSCNTSTITQACPKVSFEPIPIHYCAPAGFAILKCNDKKFNGTGPCTNVSTVQCTHGIRPVVSTQLLLNGGLAEEEVVIRSENFTNNAKTIIIQLKESVVINCTGAGHCNLSSTQWNNTLGQIVEKLRARFGNKTIRFNQPSGGDPEIVLHSFNCGGEFFYCNTSKLFNSTWNGTEGLDETNNGTGGNITLPCRIKQIVNMWQEVGKAMYAPPIRGQIRCSSNITGLLLTRDGGENNGTENNETETFRPGGGDMRDYWRSELYKYKVVKIEAGNRGPPTLDDQPRLCLLVASHLLFAPPPCLP

SEQ ID NO: 19 - protein, 352 aa, Artificial Sequence
  gp120 core variant L7-084CDC
EVVLENVTENFNMWKNNMVEQMREDVINLWDQSLKPCVKLTPLCVGAGSCDTSVITQACPKVTFEPIPIHHCAPAGFATLKCNDKKFNGTGPCRNVSTVQCTHGIKPVVSTQLLLNGSLAEGDIVIRSENFTDNAKTIIVQLRESVVINCTGAGHCNISSTQWNNTLGQIVEKLRAQFGNKTIRFNQPSGGDPEIVLHSFNCGGEFFYCNTSKLFNSTWNSTWGDTEMNNTEESNNNITLPCRIKQIVNMWQEVGKAMYAPPVRGQISCSSNITGLLLTRDGGENNGTENNETETFRPGGGGMRDNWRSELYKYKVVKIEAGNRGPPTLDDQPRLCLLVASHLLFAPPPCLP

SEQ ID NO: 20 - protein, 347 aa, Artificial Sequence
  gp120 core variant L7-098CDC
EVVLENVTENFNMWKNNMVEQMQEDIIGLWDQSLKPRVKLTPLCVGAGSCNTSVITQACPKVTFEPIPIHYCAPAGFAILKCNDKKFNGSGPCKNVSTVQCTHGIRPVVSTQLLLNGSLAEEEVVIRSENFTNNAKTIIVQLNESVKINCTGAGHCNLSSTQWNNTLGQIVGKLRAQFGNKTIRFNQPSGGDPEIVLHSFNCGGEFFYCNTSKLFNSTWNDTKRSNIEGNDTIILPCRIKRIINTWQEVGKAMYTPPVRGQISCSSNITGLLLTRDGGENNGTENNETETFRPGGGDMGDNWRSELYKYKVVKIEAGNRGPPTLDDQPRLCLLVASHLLFAPPPCLP

SEQ ID NO: 21 - protein, 345 aa, Artificial Sequence
  gp120 core variant L7-105CDC
EVVLENVTENFNMWKNNMVEQMHEDIIGLWDQSLKPCVKLTPLCVGAGSCNTSTITQACPKVSFEPIPIHYCAPAGFAILKCNDKKFNGTGPCKNVSTVQCTHGIRPVVSTQLLLNGSLAEEDIVIRSENFTDNAKTIIVQLNESVVINCTGAGHCNISSTQWNNTLGQIVGKLRAQFGNKTIRFNQPSGGDPEIVLHSFNCGGEFFYCNTSKLFNSTWNDTKRSNIDGTITLPCRIKQIVNMWQEVGKAMYAPPIRGQIRCSSNITGLLLTRDGGENNGTENNETETFRPEGGDMRDYWRSELYKYKVVKIEAGNRGPPTLDDQPRLCLLVASHLLFAPPPCLP

SEQ ID NO: 22 - protein, 32 aa, Artificial Sequence
  CDC tail polypeptide
AGNRGPPTLDDQPRLCLLVASHLLFAPPPCLP

SEQ ID NO: 23 - DNA, 1464 bp, Artificial Sequence
  ST-008 coding sequence
ggtgctgcagaacaattgtgggtcacagtctattttggggtacctgtgtgggaagaagcaaccaccactctattttgtgcatcagatgccaaagcatatgatacagaggtacataatatttgggccacacatgcctgtgtacccacagaccccaacccacaagaagtagtactagaaaatgtaacagaaaatttcaacatgtggaaaaataacatggtagatcagatgcatgaggatataatcagtttatgggatcaaagcttaaaaccatgtataaaattaatcccactctgtgttactttagattgcattgatgtggggaatgctactaatgggggggatactactaacaccactagtagtagcggggtaagaatggagccaggagaaataaaaaactgctctttcaatatcaccacaagtataagagataagatgcagaaagaatatgcacttctttataaacttgatttagtaccaatagacaatggtaatagtaatggtagtaatactaccaactataggttgataagttgtaacacctcagtcatcacacaagcctgtcccaaggtaacctttgagccaattcccatacattattgtgctccggctggttttgcgattctaaagtgtaataataagacgttcaatggaacaggaccatgtagaaatgtcagcacagtacaatgtacacatggaattaggccagtagtatcaactcaactgctgttaaatggcagtctagcagaagaagaggtagtaattagatctgaaaatttctcggacaatgctaaaaccataatagtacagctgaatgaatctgtaaaaattaattgtacaagacccaacaacaatacaagaaaaagtatacatataggaccagggagagcattttatacaacaggagaaataataggagatataagacaagcacattgtaacattagtagagcacaatggaataacactttaaaacagatagttgaaaaattaagagaacaatttaataataaaacaatagtctttactcactcctcaggaggggatccagaaattgtaatgcacagttttaattgtggaggggaatttttctactgtaattcaacacaactgtttaatagtacttggaatgatactgaaaagtcaagtggcactgaaggaaatgacaccatcatactcccatgcagaataaaacaaattataaacatgtggcaggaagtgggaaaagcaatgtatgctcctcccattaaaggacaaattagatgttcatcaaatattacagggctgctattaacaagagatggtggtaaaaatgagagtgagatcgagatcttcagacctggaggaggagatatgagggacaattggagaagtgaattatataaatataaagtagtaaaaattgaaccattaggagtagcacccaccaaggcaaagagaagagtggtgcagagagaaaaaaga

SEQ ID NO: 24 - DNA, 1434 bp, Artificial Sequence
  ST-051 coding sequence
agtgctgtagaaaagttgtgggtcacagtctattatggggtacctgtgtggaaagaagcaaccaccactctattttgtgcatcagatgctaaagcatatgatgcagaggcacataatgtctgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaaaccatgtgtaaaattaaccccactttgtgttactttaaattgcactaattatgtgaaaattgatactaatgctactgtgtataactgggaaaagatggaggaaggagaaataaaaaactgctctttcaatgtcaccacagccataagagataaggtgcagaaagaatatgcacttttttataaacttgatgtagtaccaatagaggaaaatggtaatacaagctataggttgataaattgtaacacatcaatcattacacaggcctgtccaaaggtatcctttgagccaattccaatacattattgtgccccggctggttttgcgattctaaagtgtaataataagacgttcaatggatcaggaccatgtaaaaatgtcagcacagtacagtgtacacatggaattaggccagtagtgtcaactcaactgctattaaatggcagtctagcagaagaagaggtaatacttagatctgaaaatttcacaagcaatgctaaaaccataatagtacagctgaatgaaactataaaaattaattgtacaagacctaacaacaatacaagaaaaggtatacatattggaccagggagagcattctatacaacaggacaaataataggagatataagaaaagcacattgtaaccttagtagagcacaatggaataacactttaaaacagatagttgaaaaattaagagaacaatttaataataaaacaatagtctttactcactcctcaggaggggatccagaaattgtaatgcacagttttaattgtggaggggaatttttctactgtaattcaacacaactgtttaatagtacttggaatgatactgaaaagtcaagtggcactgaaggaaatgacaccatcatactcccatgcagaataaaacaaattgtaaacatgtggcaggaagtaggcaaagcaatgtatgcccctcccatcagaggaaaaatttactgtagatcaaatattacagggctgctattaacaagagatggtggtaaaaatgagagtgagatcgagatcttcagacctggaggaggagacatgagggacaattggagaagtgaattatataaatataaagtagtaaaaattgaaccattaggaatagcacccaccaaggcaaagggaagagtggtgcagagagaaaaaaga

SEQ ID NO: 25 - DNA, 1437 bp, Artificial Sequence
  ST-080 coding sequence
agtgctacagaaagattttgggtcacagtctattatggggtacctgtgtggaaagaaacaaccaccactctattttgtgcatcagatgctaaagcatatgatacagaggtacataatgtttgggccacacatgcctgtgtacccacggaccccaacccacaagaagtagtattggaaaatgtgacagaaaattttaacatgtgggacaataacatggtagaccagatgaatgaggatataatcagtttatgggatcaaagcctaaagccatgtgtaacaataacaccactctgtgtgactttacattgcactaattatgtgaaaattgatactaatgctactgtgtataactgggaaaagatggaggaaggagaaataaaaaactgctctttcaatgtcaccacagccataagagataaggtgcagaaagaatatgcacttttttataaacttgatgtagtaccaatagaggaaaatggtaatacaagctataggttgataaattgtaacacctcagtcattacacaagcctgtccaaaggtatcctttgaaccaattcccatacattattgtgccccggctggttttgcgattctaaagtgtaatgataagaagttcaatggaaaaggaccgtgtacaaatgttagcacagtacaatgtacacatggaattaggccagtagtatcaactcaactgctgctaaatggcagtctagcagaagaaaaggttgtaattagatctgacaattttacggacaatgctaaaaccataatagtacagctgaatgaatctgtaaaaattaattgtacaaggcccagcaacaatacaagaaaaagtatacatataggaccagggagagcatcttatacaacaggagaaataataggagatataagacaagcacattgtaacattagtagagcacaatggaataacactttaaaacagatagttgaaaaattaagagaacaatttaataataaaacaatagtctttactcactcctcaggaggggatccagaaattgtaatgcacagttttaattgtggaggggaatttttctactgtaattcaacacaactgtttaatagtacttggaatgatactgaaaagtcaagtggcactgaaggaaatgacaccatcatactcccatgcagaataaaacaaattataaacatgtggcaggaagtaggcaaagcaatgtatgcccctcccatcaaaggacaaattagatgttcatcaaatattacagggctgctactaacaagggatggaggtaatagtactgagactgagactgagatcttcagacctggaggaggagatatgagggacaattggagaagtgaattatataaatataaagtagtaaaaattgaaccattaggagtagcacccaacaaggcaaagagaagagtggtgcaaagagagaaaaga

SEQ ID NO: 26 - DNA, 1446 bp, Artificial Sequence
  ST-140 coding sequence
agtgctgaagaaaacttgtgggtcacagtctattatggggtacctgtgtggaaagaagcagacaccactctattttgtgcatcagatgctaaagcatatgatagagaggtgcataatgtctgggccacacatgcctgtgtacccacagaccccaacccacaagaagtagtattgggaaatgtgacagaaaattttaacatgtggaaaaatagcatggtagatcagatgcgtgaggatataatcagtttatgggatcaaagcctaaaaccatgtgtaaaattaaccccactctgtgttactttaaattgcactaattatgtgaaaattgatactaatgctactgtgtataactgggaaaagatggaggaaggagaaataaaaaactgctctttcaatgtcaccacagccataagagataaggtgcagaaagaatatgcacttttttataaacttgatgtagtaccaatagaggaaaatggtaatacaagctataggttgataaattgtaacacatcaatcattacacaagcctgtccaaaggtatcctttgaaccaattcccatacattattgtgcaccggctggttttgcgatactaaagtgtaataataagacattcaatggaacaggaccatgtacaaatgtcagcacagtacaatgtacacatggaattaggccagtagtgtcaactcaactgctgctaaatggcagtctagcagaagaaaaggttgtaattagatctgacaattttacggacaatgctaaaaccataatagtacagctgaatgaatctgtaaaaattaattgtacaaggcccagcaacaatacaagaaaaagtatacatataggaccagggagagcattttatacaacaggagaaataataggagatataagacaagcacattgtaacattagtagagcacaatggaataacactttaaaacagatagttgaaaaattaagagaacaatttaataataaaacaatagtctttactcactcctcaggaggggatccagaaattgtaatgcacagttttaattgtggaggggaatttttctactgtaattcaacacaactgtttaatagtacttggaatgatactgaaaagtcaagtggcactgaaggaaatgacaccatcatactcccatgcagaataaaacaaattgtaaacatgtggcaggaagtaggcaaagcaatgtatgccccgcccatcagcggacagatcagatgttcgtcaaatattacagggctgttattaacaagagatggtggtgagaataatggaactgagaataacgaaactgagaccttcggacctggaggaggagatatgagggacaattggagaagtgaattatataaatataaagtagtaaaaattgaaccattaggagtagcacccaccagggcaaagagaagagtggtgcagagagaaaaaaga

SEQ ID NO: 27 - DNA, 1482 bp, Artificial Sequence
  ST-148 coding sequence
agtgttgcagaaaaaatgtgggtcacagtctattatggggtacctgtgtggaaagaagcaaccaccactctattttgtgcatcagatgctaaagcatatgatacagaggtacataatgtttgggccacacatgcctgtgtacccacagaccccaacccacaagaagtagtattgaaaaatgtgacagaaaattttaatatgtggaaaagtaacatggtagaacagatgcatgaggatataatcagtttatgggatcaaagcctaaagccatgtgtaaaattaaccccactctgtgttactttaaattgcactaatttgaatatcactaggaatactactaatcccactagtagcagctggggaatgatggagaaaggagaaataaaaaactgctctttcaatgtcaccacagccataagagataaggtgcagaaagaatatgcacttttttataaacttgatgtagtaccaatagaggaaaatggtaatacaagctataggttgataaattgtaatacctcaaccattacacaggcctgtccaaaggtatcctttgagccaattccaatacattattgtgccccggctggttttgcgatactaaagtgtaataataagacgttcaatggaacaggaccatgtacaaatgtcagcacagtacagtgtacacatggaattaggccagcagtgtcaactcaactgttgttaaatggcagtctagcagaagaagaggtagtaattagatctgaaaatttctcggacaatgctaaaactataatagtacagctgaaccaatctgtagaaattaattgtacaagacccaacaacaatacaagaagaagtataaatataggaccagggagagcactttatgcaacaggagaaataataggggatataagacaagcacattgtaacattagtgaaacacaatggaataacactttaaaacagatagttaaaaaattaagagaacaatttaagaataaaacaatagcctttaatcaatcctcaggaggggacccagaaattgtaatgcacagttttaattgtggaggggaatttttctactgtaatacaacacagttattcaatagtacttggtatcggaatggtacttggtattggaaaggtactaggattgagaatggtactgaagggttaaatgacacaattatcactctcccatgcagaataaaacaaattgtaaacatgtggcaggaagtaggaaaagcaatgtatgcccctcccatcaaaggacaaattagatgttcgtcaaatattacagggctgttattaacaagagatggtggtgagaataatggaactgagaataacgaaactgagactttcagacctggaggaggaaatatgagggacaattggagaagtgaattatataaatataaagtagtaaaaattgaaccaataggagtagcacccaccagggcaaagagaagaacagtgcaaagagaaaaaaga

SEQ ID NO: 28 - DNA, 1458 bp, Artificial Sequence
  ST-161 coding sequence
agtgctgtagaacaattgtgggtcacagtctattatggggtacctgtgtggaaagaagcaactaccactctattttgtgcatcagatgctaaagcatataagacagaggtacacaatgtttgggccacacatgcctgcgtacccacagaccccaacccacaagaagtagtattgaaaaatgtgacagaaaattttaacatgtgggacaataacatggtagaccagatgaatgaggatataatcagtttatgggatcaaagcctaaagccatgtgtaaaattaaccccactttgtgttactttaaattgcactaatgtgaagaaaggtactaatgccaccaatagtagtgaggaaatgagagagagaggggaaataaaaaactgctctttccatgtcacctcaagcataaaagataagacaacgacagagcatgcacttttttataaacttgatgtggtgccaatagatgctagaaataatagtgaaaatggtagctatatattgataagttgtaatacctcaaccattacacaggcctgtccaaaggtatcctttgagccaattccaatacattattgtgccccagctggttttgcaattctaaagtgtaatgataagacattcaatggatcaggaccatgtaaaaatgtcagcacagtacaatgtacacatggaattaggccagtagtatcaactcaactgctgctaaatggcagtctagcagaagaaaaggttgtaattagatctgacaattttacggacaatgctaaaaccataatagtacagctgaatgaatctgtaaaaattaattgtacaaggcccagcaacaatacaagaaaaagtatacatataggaccagggagagcattttatacaacaggagaaataataggagatataagacaagcacattgtaacattagtagagcacaatggaataacactttaaaacagatagttgaaaaattaagagaacaatttaataataaaacaatagtctttactcactcctcaggaggggatccagaaattgtaatgcacagttttaattgtggaggggaatttttctactgtaattcaacacaactgtttaatagtacttggaatgatactgaaaagtcaagtggtactgaagggttaaatgacacaattatcactctcccatgcaggataaaacaaattgtaaacatgtggcaggaagtaggcaaagcaatgtatgccccgcccatcagcggacagatcagatgttcgtcaaatattacagggctgctattaacaagagatggtggtgagaataatggaactgagaataacgaaactgagactttcagacctggaggaggagatatgagggacaattggagaagtgaattatataaatataaagtagtaaaaattgaaccattaggagtagcacccaccaaggcaaagagaagagtggtgcagagagaaaaaaga

SEQ ID NO: 29 - DNA, 1437 bp, Artificial Sequence
  ST-188 coding sequence
agtgttgcagaaaaaatgtgggtcacagtctattatggggtacctgtgtggagagaagcaaccaccactctattttgtgtatcagatgctaaagcatatgatacagaggtacataatgtttgggccacacatgcctgtgtacccacagaccccaacccacaagaagtagtattgaaaaatgtgacagaaaattttaacatgtggaaaaataacatggtagatcagatgcatgaggatataatcagtttatgggatgaaagcctaaagccatgtgtaaaattaaccccactctgtgttactttaaattgcactaattatgtgaaaattgatactaatgctactgtgtataactgggaaaagatggaggaaggagaaataaaaaactgctctttcaatgtcaccacagccataagagataaggtgcagaaagaatatgcacttttttataaacttgatgtagtaccaatagaggaaaatggtaatacaagctataggttgataaattgtaacacatcaatcattacacaggcttgtccaaaggtaacctttgaaccaattcccatacattattgtgccccggctggttttgcgattctaaagtgtaacaataagacgttcaatggaacaggaccatgtacaaatgtcagcacagtacaatgtacacatggaattaggccagtggtgtcaactcaactgctgttaaatggcagtctagcagaagaagaggtagtaattaggtctgaaaatttcacaaacaatgctaaaaccataataatacagctgaaggaatctgtagtaattaattgtacaagacccaataacaatacaaggaaaaggatatctataggaccagggagagtatggtatacaacaggacaaataataggagatataagaaaagcacattgtaaccttagtagcacacaatggaataacaccttaggacagatagttgaaaaattaagagcacaatttgggaataaaacaataagatttaatcaaccctcaggaggagacccagaaattgtattgcacagttttaattgtggaggggagtttttctactgtaatacatcaaaactgtttaatagtacttggaatgatactaaaaggtcaaacattgaaggaactatcacactcccatgcagaataaaacaatttataaacatgtggcagacagtaggaaaagcaatgtatgctcctcccatcaaaggacaaattagatgttcatcaaaaatttcagggctactattaacaagagatggtggtgagaataatggaactgagaataacgaaattgagactttcagacctggaggaggagacatgagggacaattggagaagtgaattatataaatataaagtagtaagaattgaaccaataggagtagcacccaccagggcaaagagaagaacagtgcaaagagagaaaaga

SEQ ID NO: 30 - DNA, 1035 bp, Artificial Sequence
  L7-010CDC coding sequence
gaagtagtattggaaaatgtgacagaaaattttaatatgtggaaaaataacatggtagaacagatgcatgaggatataatcagtttatgggatcaaagcctaaaaccatgtgtaaaattaaccccactctgtgttggagctggaagttgtaatacctcaaccattacacaggcctgtccaaaggtatccttcgagccaattccaatacattattgtgccccggctggttttgcaattctaaagtgtaatgataagaagttcaatggatcaggaccatgtaaaaatgtcagcacagtacaatgtacacatggcattaagccagtagtgtcaactcaactgctgctaaatggcagtctagcagaagaagacatagtaattagatctgaaaatttcacagacaatgctaaaaccataatagtacagctaaatgaatctgtagtaattaattgtacaggagctggacattgtaaccttagtagcacacaatggaataacaccttaggacagatagttgaaaaattaagagcacaatttgggaataaaacaataagatttaatcaaccctcaggaggagacccagaaattgtattgcacagttttaattgtggaggggagtttttctactgtaatacatcacaactgtttaatagtacttggaatgatactaaaaggtcaaacattgacggaactatcacactcccatgcagaataaaacaaattgtaaacatgtggcaggaagtaggaaaagcaatgtatgcccctcccattagaggacaaattaggtgttcatcaaatattacagggctgctattaacaagagatggtggtgagaataatggaactgagaataacgaaactgagactttcagacctggaggaggaggcatgagggacaattggagaagtgaattatataaatataaagtagtaaaaattgaagccggtaaccgaggcccaccgactctagatgatcagcctcgactgtgccttctagttgccagccatctgttgtttgcccctcccccgtgccttcct

SEQ ID NO: 31 - DNA, 1035 bp, Artificial Sequence
  L7-028CDC coding sequence
gaagtagtattggaaaatgtgacagaaaattttaacatgtggaaaaataacatggtagaacagatgcatgaggatataatcagtttatgggatcaaagcctaaaaccatgtgtaaaattaaccccactctgcgttggagctggaagttgtaacacctcaaccattacacaggcctgtccaaaggtatcctttgagccaattccaatacattattgtgccccggctggttttgcgattctaaagtgtaatgataagaagttcaatggaacaggaccatgcagaaatgtcagcacagtacaatgtacacatggaattaggccagtagtgtcaactcaactgctattaaatggcagtccagcagaagaagaggtagtaattagatctgaaaatttcacagacaatgctaaaaccataatagtacagctaaatgaatctgtagtaattaattgtacaggagctggacattgtaacattagtagcacacaatggaataacaccttaggacagatagttggaaaattaagagcacaatttgggaataaaacaataagatttaatcaaccctcaggaggagacccagaaattgtattgcacagttttaattgtggaggggagtttttctactgtaatacatcaaaactgtttaatagtacctggaatgatactaaaaggccaaacattgacggaactatcacactcccatgcagaataaaacaaattgtaaacatgtggcagggagtgggaaaagcaatgtacgcccctcccatcagaggacaaattagctgttcatcaaatattacagggctgctattaacaagagatggtggtgagaataatggaactgagaataacgaaactgagactttcagacctggaggaggagatatgagggactattggagaagcgaattatataaatataaagtagtaaaaattgaagccggtaaccgaggcccaccgactctagatgatcagcctcgactgtgccttctagttgccagccatctgttgtttgcccctcccccgtgccttcct

SEQ ID NO: 32 - DNA, 1032 bp, Artificial Sequence
  L7-043CDC coding sequence
gaagtagtattggaaaatgtggcagaaaattttaacatgtggaaaaataacatggtagaacagatgcatgaggatataatcggtttatgggatcaaagcctaaagccatgtgtaaaattaaccccactctgtgttggggctggaagttgtaacacctcaaccattacacaggcccgtccaaaggtatccttcgagccaattccaatacattattgtgccccggctggttttgcgattctaaagtgtaacgataagaagttcaatggaacaggaccatgtaaaaatgtcagcacagtacaatgtacacatggaattaggccagtagtatcaactcaactgctgctaaatggcagtctagcagaagaagaggtagtaattagatctgaaaatttctcggacaatgctaaaaccataatagtacagctaaatgaatctgtagtaattaattgtacaggagctggacattgtaacattagtagcacacaatggaataacaccttaggacagatagttgaaaaattaagagcacaatttgggaataaaacaataagatttaatcaaccctcaggaggagacccagaaattgtattgcacagttttaattgtggaggggaatttttctactgtaatacatcaaaactgtttaatagtacttggaatgatactaaaaggtcaaacattgacggaactatcacactcccatgcagaataaaacaaattgtaaacatgtggcaggaagtaggcaaagcaatgtatgctcctcccattaaaggacaaattagatgttcatcaaatattacagggctgctattaacaagagatggtggtgagaataatggaactgagaataacgaaactgagactttcagacctggaggagatatgagggacaattggagaagtgaattatataaatataaagtagtaaaaattgaagccggtaaccgaggcccaccgactctagatgatcagcctcgactgtgccttctagttgccagccatctgttgtttgcccctcccccgtgccttcct

SEQ ID NO: 33 - DNA, 1050 bp, Artificial Sequence
  L7-068CDC coding sequence
gaagtagtattggaaaatgtgacagaaaattttaacatgtgggacaataacatggtagaacagatgcatgaggatgtaatcaatttatgggatcaaagcttaaagccatgtgtaaaattaaccccactctgtgttggagctggaagttgtaacacctcaaccattacacaggcctgtccaaaggtatccttcgagccaattccaatacattattgtgccccggctggttttgcgattctaaagtgtaatgataagaagttcaatggaacaggaccatgtacaaatgtcagcacagtacagtgtacacatggaattaggcctgtagtatcaactcaactgctgttaaatggcggtctagcagaagaagaggtagtaattagatctgaaaatttcacaaacaatgctaaaaccataataatacagctgaaggaatctgtagtaattaattgtacaggagctggacattgtaaccttagtagcacacaatggaataacaccttaggacagatagttgaaaaattaagagcacgatttgggaataaaacaataagatttaatcaaccctcaggaggagacccagaaattgtattgcacagttttaattgtggaggggagtttttctactgtaatacatcaaaactgtttaatagtacttggaatggtactgaagggttagatgaaacaaataatggcactggaggaaacatcacactcccatgcagaataaaacaaattgtaaacatgtggcaggaagtaggaaaagcaatgtacgcccctcccatcagaggacaaattaggtgttcatcaaatattacagggctgttattaacaagagatggtggtgagaataatggaactgagaataacgaaactgagactttcagacctggaggaggagatatgagggactattggagaagtgaattatataaatataaagtagtaaaaattgaagccggtaaccgaggcccaccgactctagatgatcagcctcgactgtgccttctagttgccagccatctgttgtttgcccctcccccgtgccttcct

SEQ ID NO: 34 - DNA, 1056 bp, Artificial Sequence
  L7-084CDC coding sequence
gaagtagtattggagaatgtgacagaaaattttaacatgtggaaaaataacatggtagaacagatgcgggaggatgtaatcaatttatgggatcaaagcctaaagccatgtgtaaaattaaccccactctgtgttggagctggaagttgtgacacctcagtcatcacacaagcctgtcccaaggtaacctttgagccaattccaatacaccattgtgccccggctggttttgcgactctaaagtgtaatgataagaagttcaatggaacaggaccatgtagaaatgtcagcacagtacagtgtacacatggcattaagccagtagtatcaactcaactgctattaaatggcagtctagcagaaggagacatagtaattagatctgaaaatttcacagacaatgctaaaaccataatagtacagctgagggaatctgtagtaattaattgtacaggagctggacattgtaacattagtagcacacaatggaataacaccttaggacagatagttgaaaaattaagagcacaatttgggaataaaacaataagatttaatcaaccctcaggaggagacccggaaattgtgttgcacagttttaattgtggaggggagtttttctactgtaatacatcaaaactgtttaatagtacttggaatagtacttggggtgatactgaaatgaacaatactgaagagtcaaataacaatatcacactcccatgcagaataaaacaaattgtaaacatgtggcaggaagtaggcaaagcaatgtacgcccctcccgtcaggggacaaattagctgttcatcaaatattacagggctgctattaacaagagatggtggtgagaataatggaactgagaataacgaaactgagactttcagacctggaggaggaggcatgagggacaattggagaagtgaattatataaatataaagtagtaaaaattgaagccggtaaccgaggcccaccgactctagatgatcagcctcgactgtgccttctagttgccagccatctgttgtttgcccctcccccgtgccttcct

SEQ ID NO: 35 - DNA, 1041 bp, Artificial Sequence
  L7-098CDC coding sequence
gaagtagtattggaaaatgtgacagaaaattttaacatgtggaaaaataacatggtagaacagatgcaggaggatataatcggtttatgggatcaaagcctaaaaccacgtgtaaaattaaccccactctgtgttggagctggaagttgtaacacctcagtcatcacacaagcctgtcccaaggtaacctttgagccgattccaatacattattgtgccccggctggttttgcgattctaaagtgtaacgataagaagttcaatggatcaggaccatgtaaaaatgtcagcacggtacaatgtacacatggaattaggcctgtagtatcaactcaactgctgttaaatggcagtctagcagaagaagaggtagtaattaggtctgaaaatttcacaaacaatgctaaaaccataatagtacagctgaatgaatctgtaaaaattaattgtacaggagctggacattgtaaccttagtagcacacaatggaataacaccttaggacagatagttggaaaattaagagcacaatttgggaataaaacaataagatttaatcaaccctcaggaggagacccagaaattgtattgcacagttttaattgtggaggggagtttttctactgtaatacatcaaaactgtttaatagtacttggaatgatactaaaaggtcaaacattgaaggaaatgacaccatcatactcccatgcagaataaaacgaattataaacacgtggcaggaagtgggaaaagcaatgtacacccctcccgtcagaggacaaattagctgttcatcaaatattacagggctgctattaacaagagatggtggtgagaataatggaactgagaataacgaaactgagactttcagacctggaggaggagacatgggggacaattggagaagtgaattatataaatataaagtagtaaaaattgaagccggtaaccgaggcccaccgactctagatgatcagcctcgactgtgccttctagttgccagccatctgttgtttgcccctcccccgtgccttcct

SEQ ID NO: 36 - DNA, 1035 bp, Artificial Sequence
  L7-105CDC coding sequence
gaagtagtattggaaaatgtgacagaaaattttaacatgtggaaaaataacatggtagaacagatgcatgaggatataatcggtttatgggatcaaagcctaaagccatgtgtaaaattaaccccactctgtgttggagctggaagttgtaacacctcaaccattacacaggcctgtccaaaggtatccttcgagccaattccaatacattattgtgccccggctggttttgcgattctaaagtgtaacgataagaagttcaatggaacaggaccatgtaaaaatgtcagcacagtacaatgtacacatggaattaggccagtagtatcaactcaactgctgctaaatggcagtctagcagaagaagacatagtaattagatctgaaaatttcacagacaatgctaaaaccataatagtacagctaaatgaatctgtagtaattaattgtacaggagctggacattgtaacattagtagcacacaatggaataacaccttaggacagatagttggaaaattaagagcacaatttgggaataaaacaataagatttaatcaaccctcaggaggagacccagaaattgtattgcacagttttaattgtggaggggagtttttctactgtaatacatcaaaactgtttaatagtacttggaatgatactaaaaggtcaaacattgacggaactatcacactcccatgcagaataaaacaaattgtaaacatgtggcaggaagtaggcaaagcaatgtacgcccctcccatcagaggacaaattaggtgttcatcaaatattacagggctgttattaacaagagatggcggtgagaataatggaactgagaataacgaaactgagactttcagacctgaaggaggagatatgagggactattggagaagtgaattatataaatataaagtagtaaaaattgaagccggtaaccgaggcccaccgactctagatgatcagcctcgactgtgccttctagttgccagccatctgttgtttgcccctcccccgtgccttcct

SEQ ID NO: 37 - DNA, 1464 bp, Artificial Sequence
  Syn-ST-008 mammalian codon-optimized coding sequence
ggcgccgccgagcagctgtgggtgaccgtgtacttcggcgtgcccgtgtgggaggaggccaccaccaccctgttctgcgccagcgacgccaaggcctacgacaccgaggtgcacaacatctgggccacccacgcctgcgtgcccaccgaccccaacccccaggaggtggtgctggagaacgtgaccgagaacttcaacatgtggaagaacaacatggtggaccagatgcacgaggacatcatcagcctgtgggaccagagcctgaagccctgcatcaagctgatccccctgtgcgtgaccctggactgcatcgacgtgggcaacgccaccaacggcggcgacaccaccaacaccaccagcagcagcggcgtgcgcatggagcccggcgagatcaagaactgcagcttcaacatcaccaccagcatccgcgacaagatgcagaaggagtacgccctgctgtacaagctggacctggtgcccatcgacaacggcaacagcaacggcagcaacaccaccaactaccgcctgatcagctgcaacaccagcgtgatcacccaggcctgccccaaggtgaccttcgagcccatccccatccactactgcgcccccgcaggcttcgccatcctgaagtgcaacaacaagaccttcaacggcaccggcccctgccgcaacgtgagcaccgtgcagtgcacccacggcatccgccccgtggtgagcacccagctgctgctgaacggcagcctggccgaggaggaggtggtgatccgcagcgagaacttcagcgacaacgccaagaccatcatcgtgcagctgaacgagagcgtgaagatcaactgcacccgccccaacaacaacacccgcaagagcatccacatcggccccggccgcgccttctacaccaccggcgagatcatcggcgacatccgccaggcccactgcaacatcagccgcgcccagtggaacaacaccctgaagcagatcgtggagaagctgcgcgagcagttcaacaacaagaccatcgtgttcacccacagcagcggcggcgaccccgagatcgtgatgcacagcttcaactgcggcggcgagttcttctactgcaacagcacccagctgttcaacagcacctggaacgacaccgagaagagcagcggcaccgagggcaacgacaccatcatcctgccctgccgcatcaagcagatcatcaacatgtggcaggaggtgggcaaggccatgtacgccccccccatcaagggccagatccgctgcagcagcaacatcaccggcctgctgctgacccgcgacggcggcaagaacgagagcgagatcgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgc

SEQ ID NO: 38 - DNA, 1434 bp, Artificial Sequence
  Syn-ST-051 mammalian codon-optimized coding sequence
agcgccgtggagaagctgtgggtgaccgtgtactacggcgtgcccgtgtggaaggaggccaccaccaccctgttctgcgccagcgacgccaaggcctacgacgccgaggcccacaacgtgtgggccacccacgcctgcgtgcccaccgaccccaacccccaggaggtggtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccaactacgtgaagatcgacaccaacgccaccgtgtacaactgggagaagatggaggagggcgagatcaagaactgcagcttcaacgtgaccaccgccatccgcgacaaggtgcagaaggagtacgccctgttctacaagctggacgtggtgcccatcgaggagaacggcaacaccagctaccgcctgatcaactgcaacaccagcatcatcacccaggcctgccccaaggtgagcttcgagcccatccccatccactactgcgcccccgccggattcgccatcctgaagtgcaacaacaagaccttcaacggcagcggcccctgcaagaacgtgagcaccgtgcagtgcacccacggcatccgccccgtggtgagcacccagctgctgctgaacggcagcctggccgaggaggaggtgatcctgcgcagcgagaacttcaccagcaacgccaagaccatcatcgtgcagctgaacgagaccatcaagatcaactgcacccgccccaacaacaacacccgcaagggcatccacatcggccccggccgcgccttctacaccaccggccagatcatcggcgacatccgcaaggcccactgcaacctgagccgcgcccagtggaacaacaccctgaagcagatcgtggagaagctgcgcgagcagttcaacaacaagaccatcgtgttcacccacagcagcggcggcgaccccgagatcgtgatgcacagcttcaactgcggcggcgagttcttctactgcaacagcacccagctgttcaacagcacctggaacgacaccgagaagagcagcggcaccgagggcaacgacaccatcatcctgccctgccgcatcaagcagatcgtgaacatgtggcaggaggtgggcaaggccatgtacgccccccccatccgcggcaagatctactgccgcagcaacatcaccggcctgctgctgacccgcgacggcggcaagaacgagagcgagatcgagatcttccgccccggcggcggcgacatgcgcgacaactggcgcagcgagctgtacaagtacaaggtggtgaagatcgagcccctgggcatcgcccccaccaaggccaagggccgcgtggtgcagcgcgagaagcgc

SEQ ID NO: 39 - DNA, 1437 bp, Artificial Sequence
  Syn-ST-080 mammalian codon-optimized coding sequence
agcgccaccgagcgcttctgggtgaccgtgtactacggcgtgcccgtgtggaaggagaccaccaccaccctgttctgcgccagcgacgccaaggcctacgacaccgaggtgcacaacgtgtgggccacccacgcctgcgtgcccaccgaccccaacccccaggaggtggtgctggagaacgtgaccgagaacttcaacatgtgggacaacaacatggtggaccagatgaacgaggacatcatcagcctgtgggaccagagcctgaagccctgcgtgaccatcacccccctgtgcgtgaccctgcactgcaccaactacgtgaagatcgacaccaacgccaccgtgtacaactgggagaagatggaggagggcgagatcaagaactgcagcttcaacgtgaccaccgccatccgcgacaaggtgcagaaggagtacgccctgttctacaagctggacgtggtgcccatcgaggagaacggcaacaccagctaccgcctgatcaactgcaacaccagcgtgatcacccaggcctgccccaaggtgagcttcgagcccatccccatccactactgcgcccccgctggcttcgccatcctgaagtgcaacgacaagaagttcaacggcaagggcccctgcaccaacgtgagcaccgtgcagtgcacccacggcatccgccccgtggtgagcacccagctgctgctgaacggcagcctggccgaggagaaggtggtgatccgcagcgacaacttcaccgacaacgccaagaccatcatcgtgcagctgaacgagagcgtgaagatcaactgcacccgccccagcaacaacacccgcaagagcatccacatcggccccggccgcgccagctacaccaccggcgagatcatcggcgacatccgccaggcccactgcaacatcagccgcgcccagtggaacaacaccctgaagcagatcgtggagaagctgcgcgagcagttcaacaacaagaccatcgtgttcacccacagcagcggcggcgaccccgagatcgtgatgcacagcttcaactgcggcggcgagttcttctactgcaacagcacccagctgttcaacagcacctggaacgacaccgagaagagcagcggcaccgagggcaacgacaccatcatcctgccctgccgcatcaagcagatcatcaacatgtggcaggaggtgggcaaggccatgtacgccccccccatcaagggccagatccgctgcagcagcaacatcaccggcctgctgctgacccgcgacggcggcaacagcaccgagaccgagaccgagatcttccgccccggcggcggcgacatgcgcgacaactggcgcagcgagctgtacaagtacaaggtggtgaagatcgagcccctgggcgtggcccccaacaaggccaagcgccgcgtggtgcagcgcgagaagcgc

SEQ ID NO: 40 - DNA, 1446 bp, Artificial Sequence
  Syn-ST-140 mammalian codon-optimized coding sequence
tccgccgaggagaacctgtgggtgaccgtgtactatggcgtgcccgtgtggaaggaggccgacaccacgctgttctgcgcctccgacgccaaggcctacgaccgcgaagtgcacaacgtctgggccacccacgcctgcgtgcccaccgaccccaacccccaggaagtggtgctgggcaacgtcaccgagaacttcaatatgtggaaaaatagcatggtcgaccagatgcgcgaagacatcatctccctgtgggatcagtccctcaagccgtgcgtgaagctcacccccctgtgcgtcacgctgaactgtaccaattacgtgaagattgacaccaacgccaccgtgtacaattgggagaagatggaagagggcgaaatcaagaattgcagcttcaacgtgaccaccgcgatccgggacaaggtgcagaaagaatacgccctgttctacaaactcgacgtcgtccccattgaggagaacggcaacacgtcctaccgcctcatcaactgtaacacgagcatcattacgcaggcctgtcccaaggtgagcttcgagcccatcccgatccactactgcgcccccgccgggtttgcgatcctcaaatgtaacaataagaccttcaacggcaccggcccctgcaccaacgtcagcaccgtccagtgcacgcatggcatccggcccgtggtgtccacccagctgctgctgaatgggagcctcgccgaggagaaggtggtgatccggtccgacaatttcaccgacaacgccaagaccatcattgtccagctcaacgagtccgtgaaaatcaactgcacccgcccgagcaataatacgcgcaagagcattcacatcgggccggggcgggccttttacaccacgggggagattatcggcgatatccggcaggcgcattgtaacatcagccgcgcgcagtggaacaataccctgaagcagatcgtcgagaagctgcgggagcagttcaacaacaagacgatcgtgttcacccacagcagcggcggcgatcccgagatcgtgatgcactccttcaactgtggcggcgagttcttttactgcaacagcacccagctgttcaactccacgtggaacgacaccgagaagagcagcggcaccgagggcaacgacacgatcatcctgccctgccggattaagcagatcgtcaacatgtggcaggaggtggggaaggccatgtacgccccccccatcagcggccagatccgctgcagcagcaatatcaccgggctcctgctgacccgggacggcggggagaataacgggacggagaacaatgagaccgagaccttcggccccggcgggggcgacatgcgcgacaactggcgcagcgagctgtataagtacaaggtggtgaagatcgagccgctgggcgtggccccgacccgcgccaagcggcgcgtggtccagcgggagaagcgc

SEQ ID NO: 41 - DNA, 1482 bp, Artificial Sequence
  Syn-ST-148 mammalian codon-optimized coding sequence
agcgtggccgagaagatgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggaggtggtgctgaagaacgtgaccgagaacttcaacatgtggaagagcaacatggtggagcagatgcacgaggacatcatcagcctgtgggaccagagcctgaagccctgcgtgaagctgacccccctgtgcgtgaccctgaactgcaccaacctgaacatcacccgcaacaccaccaaccccaccagcagcagctggggcatgatggagaagggcgagatcaagaactgcagcttcaacgtgaccaccgccatccgcgacaaggtgcagaaggagtacgccctgttctacaagctggacgtggtgcccatcgaggagaacggcaacaccagctaccgcctgatcaactgcaacaccagcaccatcacccaggcctgccccaaggtgagcttcgagcccatccccatccactactgcgcccccgccggattcgccatcctgaagtgcaacaacaagaccttcaacggcaccggcccctgcaccaacgtgagcaccgtgcagtgcacccacggcatccgccccgccgtgagcacccagctgctgctgaacggcagcctggccgaggaggaggtggtgatccgcagcgagaacttcagcgacaacgccaagaccatcatcgtgcagctgaaccagagcgtggagatcaactgcacccgccccaacaacaacacccgccgcagcatcaacatcggccccggccgcgccctgtacgccaccggcgagatcatcggcgacatccgccaggcccactgcaacatcagcgagacccagtggaacaacaccctgaagcagatcgtgaagaagctgcgcgagcagttcaagaacaagaccatcgccttcaaccagagcagcggcggcgaccccgagatcgtgatgcacagcttcaactgcggcggcgagttcttctactgcaacaccacccagctgttcaacagcacctggtaccgcaacggcacctggtactggaagggcacccgcatcgagaacggcaccgagggcctgaacgacaccatcatcaccctgccctgccgcatcaagcagatcgtgaacatgtggcaggaggtgggcaaggccatgtacgccccccccatcaagggccagatccgctgcagcagcaacatcaccggcctgctgctgacccgcgacggcggcgagaacaacggcaccgagaacaacgagaccgagaccttccgccccggcggcggcaacatgcgcgacaactggcgcagcgagctgtacaagtacaaggtggtgaagatcgagcccatcggcgtggcccccacccgcgccaagcgccgcaccgtgcagcgcgagaagcgc

SEQ ID NO: 42 - DNA, 1458 bp, Artificial Sequence
  Syn-ST-161 mammalian codon-optimized coding sequence
tccgccgtggagcaactgtgggtgaccgtgtactatggcgtgcccgtgtggaaggaggccacgaccacgctgttctgcgcctccgacgccaaggcctacaagaccgaagtgcacaacgtctgggccacccacgcctgcgtgcccaccgaccccaacccccaggaagtggtgctgaaaaacgtcaccgagaacttcaatatgtgggacaataacatggtcgaccagatgaacgaagacatcatctccctgtgggatcagtccctcaagccgtgcgtgaagctcacccccctgtgcgtcacgctgaactgtaccaatgtcaagaagggcaccaacgccaccaattcctccgaggagatgcgggaacgcggcgaaatcaagaattgcagcttccacgtgacctccagcatcaaggacaagaccaccaccgaacatgccctgttctacaaactcgacgtcgtccccattgacgcccggaacaacagcgaaaatggctcctacatcctcatctcctgtaacacgagcaccattacgcaggcctgtcccaaggtgagcttcgagcccatcccgatccactactgcgcccccgccgggtttgcgatcctcaaatgtaacgacaagaccttcaacggcagcggcccctgcaagaacgtcagcaccgtccagtgcacgcatggcatccggcccgtggtgtccacccagctgctgctgaatgggagcctcgccgaggagaaggtggtgatccggtccgacaatttcaccgacaacgccaagaccatcattgtccagctcaacgagtccgtgaaaatcaactgcacccgcccgagcaataatacgcgcaagagcattcacatcgggccggggcgggccttttacaccacgggggagattatcggcgatatccggcaggcgcattgtaacatcagccgcgcgcagtggaacaataccctgaagcagatcgtcgagaagctgcgggagcagttcaacaacaagacgatcgtgttcacccacagcagcggcggcgatcccgagatcgtgatgcactccttcaactgtggcggcgagttcttttactgcaacagcacccagctgttcaactccacgtggaacgataccgagaagagctccgggaccgagggcctgaacgacaccatcatcaccctgccctgccggattaagcagatcgtcaacatgtggcaggaggtggggaaggccatgtacgccccccccatcagcggccagatccgctgcagcagcaatatcaccgggctcctgctgacccgggacggcggggagaataacgggacggagaacaatgagaccgagaccttccgccccggcgggggcgacatgcgcgacaactggcgcagcgagctgtataagtacaaggtggtgaagatcgagccgctgggcgtggccccgaccaaggccaagcggcgcgtggtccagcgggagaagcgc

SEQ ID NO: 43 - DNA, 1437 bp, Artificial Sequence
  Syn-ST-188 mammalian codon-optimized coding sequence
tccgtggccgagaagatgtgggtgaccgtgtactatggcgtgcccgtgtggcgcgaggccacgaccacgctgttctgcgtgtccgacgccaaggcctacgacaccgaagtgcacaacgtctgggccacccacgcctgcgtgcccaccgaccccaacccccaggaagtggtgctgaaaaacgtcaccgagaacttcaatatgtggaaaaataacatggtcgaccagatgcacgaagacatcatctccctgtgggatgagtccctcaagccgtgcgtgaagctcacccccctgtgcgtcacgctgaactgtaccaattacgtgaagattgacaccaacgccaccgtgtacaattgggagaagatggaagagggcgaaatcaagaattgcagcttcaacgtgaccaccgcgatccgggacaaggtgcagaaagaatacgccctgttctacaaactcgacgtcgtccccattgaggagaacggcaacacgtcctaccgcctcatcaactgtaacacgagcatcattacgcaggcctgtcccaaggtgaccttcgagcccatcccgatccactactgcgcccccgccgggtttgcgatcctcaaatgtaacaataagaccttcaacggcaccggcccctgcaccaacgtcagcaccgtccagtgcacgcatggcatccggcccgtggtgtccacccagctgctgctgaatgggagcctcgccgaggaggaggtggtgatccggtccgagaatttcaccaacaacgccaagaccatcattatccagctcaaggagtccgtggtgatcaactgcacccgcccgaacaataatacgcgcaagcgcattagcatcgggccggggcgggtgtggtacaccacggggcagattatcggcgatatccggaaggcgcattgtaacctgagcagcacccagtggaacaataccctgggccagatcgtcgagaagctgcgggcccagttcgggaacaagacgatccgcttcaatcagcccagcggcggcgatcccgagatcgtgctgcactccttcaactgtggcggcgagttcttttactgcaacacctccaagctgttcaactccacgtggaacgacaccaagcgcagcaacatcgagggcacgatcaccctgccctgccggattaagcagttcatcaacatgtggcagaccgtggggaaggccatgtacgccccccccatcaagggccagatccgctgcagcagcaagatcagcgggctcctgctgacccgggacggcggggagaataacgggacggagaacaatgagatcgagaccttccgccccggcgggggcgacatgcgcgacaactggcgcagcgagctgtataagtacaaggtggtgcgcatcgagccgatcggcgtggccccgacccgcgccaagcggcgcaccgtccagcgggagaagcgc

SEQ ID NO: 44 - DNA, 1035 bp, Artificial Sequence
  Syn-L7-010CDC mammalian codon-optimized coding sequence
gaagtggtgctggagaacgtcaccgagaacttcaatatgtggaaaaataacatggtcgagcagatgcacgaagacatcatctccctgtgggatcagtccctcaagccgtgcgtgaagctcacccccctgtgcgtcggggcggggtcctgtaacacgagcaccattacgcaggcctgtcccaaggtgagcttcgagcccatcccgatccactactgcgcccccgccgggtttgcgatcctcaaatgtaacgacaagaaattcaacggcagcggcccctgcaagaacgtcagcaccgtccagtgcacgcatggcatcaagcccgtggtgtccacccagctgctgctgaatgggagcctcgccgaggaggacatcgtgatccggtccgagaatttcaccgacaacgccaagaccatcattgtccagctcaacgagtccgtggtgatcaactgcaccggggccgggcattgtaacctgagcagcacccagtggaacaataccctgggccagatcgtcgagaagctgcgggcccagttcgggaacaagacgatccgcttcaatcagcccagcggcggcgatcccgagatcgtgctgcactccttcaactgtggcggcgagttcttttactgcaacacctcccagctgttcaactccacgtggaacgataccaagcgcagcaacatcgatggcaccatcaccctgccctgccggattaagcagatcgtcaacatgtggcaggaggtggggaaggccatgtacgccccccccatccgcggccagatccgctgcagcagcaatatcaccgggctcctgctgacccgggacggcggggagaataacgggacggagaacaatgagaccgagaccttccgccccggcgggggcgggatgcgcgacaactggcgcagcgagctgtataagtacaaggtggtgaagatcgaggccggtaaccgaggcccaccgactctagatgatcagcctcgactgtgccttctagttgccagccatctgttgtttgcccctcccccgtgccttcct

SEQ ID NO: 45 - DNA, 1035 bp, Artificial Sequence
  Syn-L7-028CDC mammalian codon-optimized coding sequence
gaagtggtgctggagaacgtcaccgagaacttcaatatgtggaaaaataacatggtcgagcagatgcacgaagacatcatctccctgtgggatcagtccctcaagccgtgcgtgaagctcacccccctgtgcgtcggggcggggtcctgtaacacgagcaccattacgcaggcctgtcccaaggtgagcttcgagcccatcccgatccactactgcgcccccgccgggtttgcgatcctcaaatgtaacgacaagaaattcaacggcaccggcccctgccgcaacgtcagcaccgtccagtgcacgcatggcatccggcccgtggtgtccacccagctgctgctgaatgggagccccgccgaggaggaggtggtgatccggtccgagaatttcaccgacaacgccaagaccatcattgtccagctcaacgagtccgtggtgatcaactgcaccggggccgggcattgtaacatcagcagcacccagtggaacaataccctgggccagatcgtcggcaagctgcgggcccagttcgggaacaagacgatccgcttcaatcagcccagcggcggcgatcccgagatcgtgctgcactccttcaactgtggcggcgagttcttttactgcaacacctccaagctgttcaactccacgtggaacgataccaagcgccccaacatcgatggcaccatcaccctgccctgccggattaagcagatcgtcaacatgtggcagggggtggggaaggccatgtacgccccccccatccgcggccagatctcctgcagcagcaatatcaccgggctcctgctgacccgggacggcggggagaataacgggacggagaacaatgagaccgagaccttccgccccggcgggggcgacatgcgcgactactggcgcagcgagctgtataagtacaaggtggtgaagatcgaggccggtaaccgaggcccaccgactctagatgatcagcctcgactgtgccttctagttgccagccatctgttgtttgcccctcccccgtgccttcct

SEQ ID NO: 46 - DNA, 1032 bp, Artificial Sequence
  Syn-L7-043CDC mammalian codon-optimized coding sequence
gaagtggtgctggagaacgtcgccgagaacttcaatatgtggaaaaataacatggtcgagcagatgcacgaagacatcatcggcctgtgggatcagtccctcaagccgtgcgtgaagctcacccccctgtgcgtcggggcggggtcctgtaacacgagcaccattacgcaggcccgccccaaggtgagcttcgagcccatcccgatccactactgcgcccccgccgggtttgcgatcctcaaatgtaacgacaagaaattcaacggcaccggcccctgcaagaacgtcagcaccgtccagtgcacgcatggcatccggcccgtggtgtccacccagctgctgctgaatgggagcctcgccgaggaggaggtggtgatccggtccgagaatttctccgacaacgccaagaccatcattgtccagctcaacgagtccgtggtgatcaactgcaccggggccgggcattgtaacatcagcagcacccagtggaacaataccctgggccagatcgtcgagaagctgcgggcccagttcgggaacaagacgatccgcttcaatcagcccagcggcggcgatcccgagatcgtgctgcactccttcaactgtggcggcgagttcttttactgcaacacctccaagctgttcaactccacgtggaacgataccaagcgcagcaacatcgatggcaccatcaccctgccctgccggattaagcagatcgtcaacatgtggcaggaggtggggaaggccatgtacgccccccccatcaagggccagatccgctgcagcagcaatatcaccgggctcctgctgacccgggacggcggggagaataacgggacggagaacaatgagaccgagaccttccgccccgggggcgacatgcgcgacaactggcgcagcgagctgtataagtacaaggtggtgaagatcgaggccggtaaccgaggcccaccgactctagatgatcagcctcgactgtgccttctagttgccagccatctgttgtttgcccctcccccgtgccttcct

SEQ ID NO: 47 - DNA, 1050 bp, Artificial Sequence
  Syn-L7-068CDC mammalian codon-optimized coding sequence
gaagtggtgctggagaacgtcaccgagaacttcaatatgtgggacaataacatggtcgagcagatgcacgaagacgtgatcaacctgtgggatcagtccctcaagccgtgcgtgaagctcacccccctgtgcgtcggggcggggtcctgtaacacgagcaccattacgcaggcctgtcccaaggtgagcttcgagcccatcccgatccactactgcgcccccgccgggtttgcgatcctcaaatgtaacgacaagaaattcaacggcaccggcccctgcaccaacgtcagcaccgtccagtgcacgcatggcatccggcccgtggtgtccacccagctgctgctgaatgggggcctcgccgaggaggaggtggtgatccggtccgagaatttcaccaacaacgccaagaccatcattatccagctcaaggagtccgtggtgatcaactgcaccggggccgggcattgtaacctgagcagcacccagtggaacaataccctgggccagatcgtcgagaagctgcgggcccgcttcgggaacaagacgatccgcttcaatcagcccagcggcggcgatcccgagatcgtgctgcactccttcaactgtggcggcgagttcttttactgcaacacctccaagctgttcaactccacgtggaacggcaccgagggcctcgacgagaccaacaacggcaccggcggcaacatcaccctgccctgccggattaagcagatcgtcaacatgtggcaggaggtggggaaggccatgtacgccccccccatccgcggccagatccgctgcagcagcaatatcaccgggctcctgctgacccgggacggcggggagaataacgggacggagaacaatgagaccgagaccttccgccccggcgggggcgacatgcgcgactactggcgcagcgagctgtataagtacaaggtggtgaagatcgaggccggtaaccgaggcccaccgactctagatgatcagcctcgactgtgccttctagttgccagccatctgttgtttgcccctcccccgtgccttcct

SEQ ID NO: 48 - DNA, 1056 bp, Artificial Sequence
  Syn-L7-084CDC mammalian codon-optimized coding sequence
gaagtggtgctggagaacgtcaccgagaacttcaatatgtggaaaaataacatggtcgagcagatgcgcgaagacgtgatcaacctgtgggatcagtccctcaagccgtgcgtgaagctcacccccctgtgcgtcggggcggggtcctgtgacacgagcgtgattacgcaggcctgtcccaaggtgaccttcgagcccatcccgatccaccactgcgcccccgccgggtttgcgaccctcaaatgtaacgacaagaaattcaacggcaccggcccctgccgcaacgtcagcaccgtccagtgcacgcatggcatcaagcccgtggtgtccacccagctgctgctgaatgggagcctcgccgagggggacatcgtgatccggtccgagaatttcaccgacaacgccaagaccatcattgtccagctccgggagtccgtggtgatcaactgcaccggggccgggcattgtaacatcagcagcacccagtggaacaataccctgggccagatcgtcgagaagctgcgggcccagttcgggaacaagacgatccgcttcaatcagcccagcggcggcgatcccgagatcgtgctgcactccttcaactgtggcggcgagttcttttactgcaacacctccaagctgttcaactccacgtggaacagcacctggggcgacaccgagatgaacaacaccgaggagtccaacaacaacatcaccctgccctgccggattaagcagatcgtcaacatgtggcaggaggtggggaaggccatgtacgccccccccgtccgcggccagatctcctgcagcagcaatatcaccgggctcctgctgacccgggacggcggggagaataacgggacggagaacaatgagaccgagaccttccgccccggcgggggcgggatgcgcgacaactggcgcagcgagctgtataagtacaaggtggtgaagatcgaggccggtaaccgaggcccaccgactctagatgatcagcctcgactgtgccttctagttgccagccatctgttgtttgcccctcccccgtgccttcct

SEQ ID NO: 49 - DNA, 1041 bp, Artificial Sequence
  Syn-L7-098CDC mammalian codon-optimized coding sequence
gaagtggtgctggagaacgtcaccgagaacttcaatatgtggaaaaataacatggtcgagcagatgcaagaagacatcatcggcctgtgggatcagtccctcaagccgcgggtgaagctcacccccctgtgcgtcggggcggggtcctgtaacacgagcgtgattacgcaggcctgtcccaaggtgaccttcgagcccatcccgatccactactgcgcccccgccgggtttgcgatcctcaaatgtaacgacaagaaattcaacggcagcggcccctgcaagaacgtcagcaccgtccagtgcacgcatggcatccggcccgtggtgtccacccagctgctgctgaatgggagcctcgccgaggaggaggtggtgatccggtccgagaatttcaccaacaacgccaagaccatcattgtccagctcaacgagtccgtgaaaatcaactgcaccggggccgggcattgtaacctgagcagcacccagtggaacaataccctgggccagatcgtcggcaagctgcgggcccagttcgggaacaagacgatccgcttcaatcagcccagcggcggcgatcccgagatcgtgctgcactccttcaactgtggcggcgagttcttttactgcaacacctccaagctgttcaactccacgtggaacgataccaagcgcagcaacatcgagggcaacgacacgatcatcctgccctgccggattaagcgcatcatcaacacctggcaggaggtggggaaggccatgtacaccccccccgtccgcggccagatctcctgcagcagcaatatcaccgggctcctgctgacccgggacggcggggagaataacgggacggagaacaatgagaccgagaccttccgccccggcgggggcgacatgggggacaactggcgcagcgagctgtataagtacaaggtggtgaagatcgaggccggtaaccgaggcccaccgactctagatgatcagcctcgactgtgccttctagttgccagccatctgttgtttgcccctcccccgtgccttcct

SEQ ID NO: 50 - DNA, 1035 bp, Artificial Sequence
  Syn-L7-105CDC mammalian codon-optimized coding sequence
gaagtggtgctggagaacgtcaccgagaacttcaatatgtggaaaaataacatggtcgagcagatgcacgaagacatcatcggcctgtgggatcagtccctcaagccgtgcgtgaagctcacccccctgtgcgtcggggcggggtcctgtaacacgagcaccattacgcaggcctgtcccaaggtgagcttcgagcccatcccgatccactactgcgcccccgccgggtttgcgatcctcaaatgtaacgacaagaaattcaacggcaccggcccctgcaagaacgtcagcaccgtccagtgcacgcatggcatccggcccgtggtgtccacccagctgctgctgaatgggagcctcgccgaggaggacatcgtgatccggtccgagaatttcaccgacaacgccaagaccatcattgtccagctcaacgagtccgtggtgatcaactgcaccggggccgggcattgtaacatcagcagcacccagtggaacaataccctgggccagatcgtcggcaagctgcgggcccagttcgggaacaagacgatccgcttcaatcagcccagcggcggcgatcccgagatcgtgctgcactccttcaactgtggcggcgagttcttttactgcaacacctccaagctgttcaactccacgtggaacgataccaagcgcagcaacatcgatggcaccatcaccctgccctgccggattaagcagatcgtcaacatgtggcaggaggtggggaaggccatgtacgccccccccatccgcggccagatccgctgcagcagcaatatcaccgggctcctgctgacccgggacggcggggagaataacgggacggagaacaatgagaccgagaccttccgccccgaagggggcgacatgcgcgactactggcgcagcgagctgtataagtacaaggtggtgaagatcgaggccggtaaccgaggcccaccgactctagatgatcagcctcgactgtgccttctagttgccagccatctgttgtttgcccctcccccgtgccttcct

SEQ ID NO: 51 - DNA, 96 bp, Artificial Sequence
  CDC tail mammalian codon-optimized coding sequence
gccggtaaccgaggcccaccgactctagatgatcagcctcgactgtgccttctagttgccagccatctgttgtttgcccctcccccgtgccttcct

SEQ ID NO: 52 - protein, 23 aa, Artificial Sequence
  tPA signal peptide sequence
MDAMKRGLCCVLLLCGAVFVSPS

SEQ ID NO: 53 - DNA, 75 bp, Artificial Sequence
  tPA signal peptide coding sequence
atggatgcaatgaagagagggctctgctgtgtgctgctgctgtgtggagcagtcttcgtttcgcccagcgctagc

SEQ ID NO: 54 - protein, 511 aa, Human Immunodeficiency Virus type 1
  HXB2 gp120 polypeptide
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 55 - protein, 28 aa, Human Immunodeficiency Virus type 1
  HXB2 gp120 signal peptide
MRVKEKYQHLWRWGWRWGTMLLGMLMIC

SEQ ID NO: 56 - protein, 489 aa, Artificial Sequence
  gp120 variant ST-003
SAVEKLWVTIYYGVPVWKEATTTLFCASDAKAYDTEVHNVWATHACVPTDPNPQEVVLGNVTENFNIWKNNMVEQMHGDIISLWDQSLKPCVKLTPLCVTLDCIDVGNATNGGNTTNTTGSGGVRMEPGEIKNCSFNITTSIRDKMQKEYALLYKLDLVPIDNGNSNGSNTTNYRLISCNTSVITQACPKVSFEPIPIHYCTPAGFAILKCNNKKFNGTGPCKNVSTVQCTHGIRPVVSTQLLLNGSLAEEEVVIRSENFSDNAKTIIVQLNESVVINCTRPNNNTRRRLSIGPGRVWYTTGEIIGDIRQAHCNISRAKWNNTLQQIVIKLREKFRNKTIAFNQSLGGDPEIVMHSFNCGGEFFYCNTAQLFNSTWNDTKRSNIEGTITLPCRIKQIVNMWQEVGKAMYAPPIKGQISCSSNITGLLLTRDGGENNGTENNETETFRPGGGDMRDNWRSELYKYKVVKIEPLGVAPTRAKRRVVQREKR

SEQ ID NO: 57 - protein, 492 aa, Artificial Sequence
  gp120 variant ST-057
SVVEQLWVTVYYGVPVWKEATTTLFCASDAKAYDTEVHNVWATHACVPTDPNPQEVVLKNVTENFNMWDNNMVDQMNEDIISLWDQSLKPCVKLTPLCVTLDCIDVGNATNGGNTTNTTSSSGVRMEPGEIKNCSFNITTSIRDKMQKEYALLYKLDLVPIDNGNSNGSNTTNYRLISCNTSVITQACPKVTFEPIPIHYCAPAGFAILKCNDKKFNGSEPCRNVSTVQCTHGIRPVVSTQLLLNGSLAEEEVVIRSENFTNNAKTIIIQLKESVKINCTRPNNNTRKRISIGPGRALYATGDIIGDIRQAHCNISETQWNNTLKQIVKKLREQFKNKTIAFNHSSGGDPEIVMHSFNCGGEFFYCNTSKLFNSTWNGSETEEVNTTDQNITLPCRIKQIVNMWQEVGKAMYAPPISGQIRCSSNITGLLLTRDGGENNGTENNETETFRPGGGDMRDNWRSELYKYKVVKIEPLGVAPTKAKRRVVQRKKR

SEQ ID NO: 58 - protein, 489 aa, Artificial Sequence
  gp120 variant ST-128
SAAKEKTWVTIYYGVPVWKEATTTLFCASDAKAYKTEVHNVWATHTCVPTDPNPQEVVLGNVTENFNMWDNNMVDQMNEDIISLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNTSKLFNSTWEFNSTWNGTEGLDETNNGTGGNITLPCRIKQIVNMWQGVGKAMYAPPISGQIRCSSNITGLLLTRDGGENNGTENNETETFRPGGGDMRDNWRSELYKYKVVKIEPLGVAPTKAKRRVVQREKR

SEQ ID NO: 59 - protein, 487 aa, Artificial Sequence
  gp120 variant ST-168
SAEENLWVTVYYGVPVWKDAITTLFCASDAKAYDREVHNVWATHACVPTDPNPQEVVLENVTEDFNMWKNNMVEQMQEDVINLWDQSLKPCVKLTPLCVTLNCIDVGNATNGGNTTNTTSSSGVRMEPGEIKNCSFNITTSIRDKMQKEYALLYKLDLVPIDNGNSNGSNTTNYRLISCNTSVITQACPKVTFEPIPIHYCAPAGFAILKCNNKTFNGTEPCRNVSTVQCTHGIKPVVSTQLLLNGSLTEDEIVIRSKNFSDNAKIIIVQLNKSVEINCTRPNNNTRKSVSLGPGSAWYATGDIIGDIRQAHCNLSLVQWNNTLRQVAIKLREQFKNKTIVFKNSTGGDPEIVLHSFNCGGEFFYCNTTKLFNSTWNGSETEEVNTTDQNITLPCRIKQFINMWQTVGKAMYAPPIKGQIRCSSKISGLLLTRDGGNTNNTEIFRPGGGDMKDNWRSELYKYKVVKIEPLGIAPNKAKRRVVQRERR

SEQ ID NO: 60 - protein, 482 aa, Artificial Sequence
  gp120 variant ST-173
SANEQTWVTVYYGVPVWKETTTTLFCASDAKAYDTEVHNVWATHACIPTDPNPQEVVLKNVTENFNMWDNNMVEQMQEDVINLWDQSLKPCVKLTPLCVTLNCKDVNATNTTSSSEGMMERGEIKNCSFNITTSIRDKMQKEYALLYKLDLVPIDNGNSNGSNTTNYRLISCNTSVITQACPKVTFEPIPIHYCAPAGFAILKCNDKKFNGTGPCTNVSTVQCTHGIRPVVSTQLLLDGSLAEEEVVIRSENFSDNAKTIIVQLNQSVEINCTRPNNNTRRSTNIGPERALYATGEIIGDIRQAHCNISRAQWNNTLRQVVEKLRAQFGNKTIRFNQPSGGDPEIVLHSFNCGGEFFYCNTSKLFNSTWNDTKRSNIEGTITLPCRIKQIVNMWQEVGKAMYAPPISGQIRCSSNITGLLLTRDGGENNGTENNETETFRPGGGDMRDNWRSELYKYKVVKIEPLGVAPTRAKRRVVQREKR

SEQ ID NO: 61 - protein, 494 aa, Artificial Sequence
  gp120 variant ST-194
SAAKEKTWVTIYYGVPVWKDAITTLFCASDAKAYDAEAHNVWATHACVPTDPNPQEVVLGNVTENFNMWKNNMVDQMHEDIISLWDQSLKPCVKLTPLCVTLNCTNYVKIDTNATVYNWEKMEEGEIKNCSFNVTTAIRDKVQKEYALFYKLDVVPIEENGNTSYRLINCNTSIITQACPKVTFEPIPIHYCAPAGFAILKCNNKTFNGKGPCTNVSTVQCTHGIRPVVSTQLLLNGSLAEKEVILRSENFTSNAKTIIVQLNETIKINCTRPNNNTRKGIHIGPGRAFYTTGEVIGNIRQAHCNISSTDWGKTLEKVVEKLRKQFGSNKTIVFNHSSGGDPEIVMHSFNCGGEFFYCNTTQLFNSTWYRNGTWYWKGTRIENGTEGLNDTIITLPCRIKQIINMWQEVGKAMYAPPISGQIRCSSNITGLLLTRDGGENNGTENNETETFRPGGGDMKDNWRSELYKYKVVKIEPLGVAPTKAKRRVVQREKR

SEQ ID NO: 62 - protein, 492 aa, Artificial Sequence
  gp120 variant ST-199
SAEENLWVTVYYGVPVWREATTTLFCASGAKAYDTEVHNVWATHACVPTDPNPQEVVLENVTENFNMWKNNMVDQMHEDIISLWDQSLKPCVKLTPLCVTLDCIDVGNATNGGNTTNTTSSSGVRMEPGEIKNCSFNITTSIRDKMQKEYALLYKLDLVPIDNGNSNGSNTTNYRLISCNTSVITQACPKVTFEPIPIHYCAPAGFAILKCNNKTFNGTGPCRNVSTVQCTHGIRPVVSTQLLLNGSLAEEEVVIRSENFSDNAKTIIVRLNQSVEINCTRPNNNTRRSINIGPGRALYATGDIIGDIRQAHCNLSLVQWNNTLRQVAIKLREQFKNKTIVFKNSTGGDPEIVLHSFNCGGEFFYCNTTKLFNSTWNGSETEEVNTTDQNITLPCRIKQFINMWQEVGKAMYAPSISGQIRCSSNITGLLLTRDGGENNGTENNETETFRPGGGDMRDNWRSELYKYKVVKIEPLGVAPTRAKRRVVQREKR

SEQ ID NO: 63 - protein, 483 aa, Artificial Sequence
  gp120 variant ST-272
SAVEQLWVTVYYGVPVWKEATTTLFCASDAKAHETEVHNVWATHACVPTDPNPQEVLLENVTENFNMWKNSMVDQMHEDIISLWDQSLKPCTKLTPLCVTLNCTDDLKNFTINTNGSIWQNMEKGEIKNCSFNITTTLKDKVQREYALFYKLDVIPIENNNSSYRLMSCNTSKITQACPKVSFEPIPIHYCTPAGFAILKCNDKKFNGTGPCKNVSTVQCTHGIRPVVSTQLLLNGSLAEEDIVIRSENFTNNAKTIIVQLNESVVINCIRPNNNTRKRISIGPGRVLYATGEIIGDIRQAHCNISSTQWNNTLGQIVEKLRAQFGNKTIRFNQPSGGDPEIVLHSFNCGGEFFYCNTSKLFNGTWYWKGTRIENGTEGLNDTIITLPCRIKQIINMWQKVGKAMYAPPISGQIRCSSNITGLLLTRDGGSSNDNDTEIFRPGGGDMRDNWRSELYKYKVVKIEPLGVAPTKAKRRVVQREKR

SEQ ID NO: 64 - DNA, 1467 bp, Artificial Sequence
  ST-003 coding sequence
agtgctgtagaaaagttgtgggtcacaatctattatggggtacctgtgtggaaagaagcaaccaccactctattttgtgcatcagatgctaaagcatatgatacagaggtacacaatgtctgggccacacatgcctgtgtacctacagaccccaacccacaagaagtagtattgggaaatgtaacagaaaattttaacatatggaaaaataacatggtagaacagatgcatggggatataatcagtttatgggatcaaagcctaaagccatgtgtaaaattaaccccactctgtgttactttagattgcattgatgtggggaatgctactaatggggggaatactactaacaccactggtagtggcggggtaagaatggagccaggagaaataaaaaactgctctttcaatatcaccacaagtataagagataagatgcagaaagaatatgcacttctttataaacttgatttagtaccaatagacaatggtaatagtaatggtagtaatactaccaactataggttgataagctgtaacacctcagtcattacacaagcctgtccaaaggtatcctttgaaccaattcccatacattattgtaccccggctggttttgcgattctaaagtgtaacaataagaagttcaatggaacaggaccatgtaaaaatgtcagcacagtacaatgtacacatggaattaggccagtagtatcaactcaactgctgctaaatggcagtctagcagaagaagaggtagtaattagatctgaaaatttctcggacaatgctaaaaccataatagtacagctaaatgaatctgtagtaattaattgtacaagacccaacaacaatacaagaagaaggttatctataggaccagggagagtatggtatacaacaggagaaataataggagatataagacaagcacattgtaacattagtagagcaaaatggaataacactttacaacagatagttataaaattaagagaaaaatttaggaataaaacaatagcctttaatcaatccttaggaggggacccagaaattgtaatgcacagttttaattgtggaggggagtttttctactgcaatacagcacaactgtttaatagtacttggaatgatactaaaaggtcaaacattgaaggaactatcacacttccatgcagaataaaacaaattgtaaacatgtggcaggaagtaggaaaggcaatgtatgctcctcccatcaaaggacaaattagctgttcgtcaaatattacagggctgttattaacaagagatggtggtgagaataatggaactgagaataacgaaactgagactttcagacctggaggaggagatatgagggacaattggagaagtgaattatataaatacaaagtagtaaaaattgaaccattaggagtagcacccaccagggcaaagagaagagtggtgcagagagaaaaaaga

SEQ ID NO: 65 - DNA, 1476 bp, Artificial Sequence
  ST-057 coding sequence
agtgttgtagaacaattgtgggtcacagtctattatggggtacccgtgtggaaagaagcaaccaccactctattttgtgcatcagatgctaaagcctatgatacagaggtacataatgtttgggccacacatgcctgtgtacccacagaccccaacccacaagaagtagtattgaaaaatgtgacagaaaattttaacatgtgggacaataacatggtagaccagatgaatgaggatataatcagtttatgggatcaaagcctaaagccatgtgtaaaattaaccccactctgtgttactttagattgcattgatgtggggaatgctactaatggggggaatactactaacaccactagtagtagcggggtaagaatggagccaggagaaataaaaaactgctctttcaatatcaccacaagtataagagataagatgcagaaagaatatgcacttctttataaacttgatttagtaccaatagacaatggtaatagtaatggtagtaatactaccaactataggttgataagttgtaacacctcagtcatcacacaagcctgtcccaaggtaacctttgagccaattcccatacattattgtgctccggctggttttgcgatactaaagtgtaatgataagaagttcaatggatcagaaccgtgtagaaatgtcagcacagtacaatgtacacatggaattaggccagtagtatcaactcaattgctgttaaatggcagtctagcagaagaagaggtagtaattagatctgaaaatttcacaaacaatgctaaaaccataataatacagctgaaggaatctgtaaaaattaattgtacaagacctaacaacaatacaaggaaaaggatatctataggaccagggagagcactttatgcaacaggagacataataggagatataagacaggcacattgtaacattagtgaaacacaatggaataacactttaaaacagatagttaaaaaattaagagaacaatttaagaataaaacaatagcctttaatcactcctcaggaggggacccagaaattgtgatgcacagttttaattgtggaggggaatttttctactgcaatacatcaaaactgtttaatagtacttggaatggaagtgagactgaagaagttaatacaactgaccaaaatatcacactcccatgcagaataaaacaaattgtaaacatgtggcaggaagtaggcaaagcaatgtatgccccgcccatcagcggacaaattagatgttcgtcaaatattacagggctgttattaacaagagatggtggtgagaataatggaactgagaataacgaaactgagactttcagacctggaggaggagatatgagggacaattggagaagtgaattatataaatataaagtggtaaaaattgaaccattaggagtagcacccaccaaggcaaagagaagagtggtgcagagaaaaaaaaga

SEQ ID NO: 66 - DNA, 1467 bp, Artificial Sequence
  ST-128 coding sequence
agtgctgcaaaagaaaaaacgtgggtcacaatctattatggggtacctgtgtggaaagaagcaaccaccactctattttgtgcatcagatgctaaagcatataagacagaggtacacaatgtttgggccacacatacctgtgtacccacagaccccaacccacaagaagtagtattgggaaatgtgacagaaaattttaacatgtgggacaataacatggtagaccagatgaatgaggatataatcagtttatgggatcaaagcctaaagccatgtgtaaaattaaccccactctgtgttactttaaattgcaaagatgtgaatgctactaataccactagtagtagtgagggaatgatggagagaggagaaataaaaaactgctctttcaatatcaccaaaagcataagagataaggtgcagaaagaatatgctcttttttataaactggatgtagtaccaatagataataagaataataccaaatataggttaataagttgtaacacctcagtcattacacaagcctgtccaaaggtatcctttgaaccaattcccatacattattgtgccccggctggttttgcgattctaaagtgtaataataagacattcaatggaaaaggacaatgtaaaaatgtcagcacagtacaatgtacacatggaattaggccagtagtatcaactcaactgctgctaaatggcagtctagcagaagaaaaggttgtaattagatctgacaattttacggacaatgctaaaaccataatagtacagctgaatgaatctgtaaaaattaattgtacaaggcccagcaacaatacaagaaaaagtatacatataggaccagggagagcattttatacaacaggagaaataataggagatataagacaagcacattgtaacattagtagagcacaatggaataacactttaaaacagatagttgaaaaattaagagaacaatttaataataaaacaatagtctttactcactcctcaggaggggatccagaaattgtaatgcacagttttaattgtggaggggaatttttctactgcaatacgtcaaaactgtttaatagtacttgggagtttaatagcacttggaatggtactgaagggttagatgaaacaaataatggcactggaggaaacatcacactcccatgcagaataaaacaaattgtaaacatgtggcagggagtaggcaaagcaatgtatgcccctcccatcagcggacagatcagatgttcgtcaaatattacagggctgttattaacaagagatggtggtgagaataatggaactgagaataacgaaactgagactttcagacctggaggaggagatatgagggacaattggagaagtgaattatataaatataaagtagtaaagattgaaccattaggagtagcacccaccaaggcaaagagaagagtggtgcaaagagagaaaaga

SEQ ID NO: 67 - DNA, 1461 bp, Artificial Sequence
  ST-168 coding sequence
agtgctgaagaaaacttgtgggtcacagtctattatggggtacctgtgtggaaagacgcaatcaccactctattttgtgcatcagatgctaaagcatatgatagagaggtacataatgtttgggccacacatgcctgtgtacccacagaccccaacccacaagaagtagtattggaaaatgtaacagaagattttaacatgtggaaaaataacatggtagaacagatgcaggaggatgtaatcaatttatgggatcaaagcttaaagccatgtgtaaaattaaccccactctgtgttactttaaattgcattgatgtggggaatgctactaatggggggaatactactaacaccactagtagtagcggggtaagaatggagccaggagaaataaaaaactgctctttcaatatcaccacaagtataagagataagatgcagaaagaatatgcacttctttataaacttgatttagtaccaatagacaatggtaatagtaatggtagtaatactaccaactataggttgataagttgtaacacctcagtcatcacacaagcctgtcccaaggtaacctttgagccaattcccatacattattgtgctccggctggttttgcgattctaaagtgtaataataagacgttcaatggaacagaaccgtgtagaaatgtcagcacagtacaatgtacacatggaattaagccagtagtatcaactcaactactgttaaatggcagtctaacagaagatgaaatagtaattagatctaaaaatttctcggacaatgctaaaatcataatagtacagctgaacaaatctgtagaaattaattgtacaaggcccaacaacaatacaagaaaaagtgtaagtctaggaccagggagcgcatggtatgcaacaggagacataataggagatataagacaagcacattgtaatcttagccttgtacaatggaacaacactttaagacaggtagctataaaattaagagaacaatttaagaataaaacaatagtctttaaaaactccacaggaggggacccagagattgtactgcacagttttaattgtggaggggaatttttctactgtaacacaacaaaactgtttaatagtacttggaatggaagtgagactgaagaagttaatacaactgaccaaaatatcacactcccatgcaggataaaacaatttataaacatgtggcagacagtaggaaaagcaatgtatgcccctcccatcaaaggacaaattagatgttcatcaaaaatttcagggctactattaacaagagatggtgggaataccaacaataccgagatcttcagacctggaggaggagatatgaaggacaattggagaagtgaattatataaatataaagtagtaaaaattgaaccattaggaatagcacccaacaaggcaaagagaagagtggtgcaaagagaaagaaga

SEQ ID NO: 68 - DNA, 1446 bp, Artificial Sequence
  ST-173 coding sequence
agtgctaatgaacaaacgtgggtcacagtctattatggggtacctgtgtggaaagaaacaaccaccactctattttgtgcatcagatgctaaagcatatgatacagaggtacataatgtttgggccacacatgcctgtatacccacagaccccaacccacaagaagtagtattgaaaaatgtgacagaaaattttaacatgtgggacaataacatggtagaacagatgcaggaggatgtaatcaatttatgggatcaaagcctaaagccatgtgtaaaattaaccccactttgtgttactttaaattgcaaagatgtgaatgctactaataccactagtagtagtgagggaatgatggagagaggagaaataaaaaactgctctttcaatatcaccacaagtataagagataagatgcagaaagaatatgcacttctttataaacttgatttagtaccaatagacaatggtaatagtaatggtagtaatactaccaactataggttgataagttgtaacacctcagtcatcacacaagcctgtcccaaggtaacctttgagccaattcccatacattattgtgccccggctggttttgcgattctaaagtgtaatgataagaagttcaatggaacaggaccatgcacaaatgtcagcacagtacaatgtacacatggaattaggccagtagtgtcaactcaactgttgttagatggcagtctagcagaagaagaggtagtaattagatctgaaaatttctcggacaatgctaaaactataatagtacagctgaaccaatctgtagaaattaattgtacaagacccaacaacaatacaagaagaagtacaaatataggaccagagagagcactttatgcaacaggagaaataataggggatataagacaagcacattgtaacattagtagagcacaatggaataacaccttaagacaggtagttgaaaaattaagagcacaatttgggaataaaacaataagatttaatcaaccctcaggaggagacccagaaattgtattgcacagttttaattgtggaggggagtttttctactgtaatacatcaaaactgtttaatagtacttggaatgatactaaaaggtcaaacattgaaggaactatcacactcccatgcagaataaaacaaattgtaaacatgtggcaggaagtaggcaaagcaatgtatgccccgcccatcagcggacagatcagatgttcgtcaaatattacagggctgttattaacaagagatggtggtgagaataatggaactgagaataacgaaactgagactttcagacctggaggaggagatatgagggacaattggagaagtgaattatataaatataaagtagtaaaaattgaaccattaggagtagcacccaccagggcaaagagaagagtggtgcagagagaaaaaaga

SEQ ID NO: 69 - DNA, 1482 bp, Artificial Sequence
  ST-194 coding sequence
agtgctgcaaaagaaaaaacgtgggtcacaatctattatggggtacctgtgtggaaagacgcaatcaccactctattttgtgcatcagatgctaaagcatatgatgcagaggcacataatgtctgggccacacatgcctgtgtacccacagaccccaacccacaagaagtagtattgggaaatgtgacagaaaattttaacatgtggaaaaataacatggtagatcagatgcatgaggatataatcagtttatgggatcaaagcctaaaaccatgtgtaaaattaaccccactctgtgttactttaaattgcactaattatgtgaaaattgatactaatgctactgtgtataactgggaaaagatggaggaaggagaaataaaaaactgctctttcaatgtcaccacagccataagagataaggtgcagaaagaatatgcacttttttataaacttgatgtagtaccaatagaggaaaatggtaatacaagctataggttgataaattgtaacacatcaatcattacacaggcttgtccaaaggtaacctttgaaccaattccaatacattattgtgccccggctggttttgcgattctaaagtgtaacaataagacgttcaatggaaaaggaccgtgtacaaatgttagcacagtacaatgtacacatggaattaggccagtagtgtcaactcaattgctgttaaatggcagtctagcagaaaaagaggtaatacttagatctgaaaatttcacaagcaatgctaaaaccataatagtacagctgaatgaaactataaaaattaattgtacaagacctaacaacaatacaagaaaaggtatacatattggaccagggagagcattctatacaacaggagaagtaataggaaatataagacaagcacattgtaacattagtagtacagactgggggaaaactctagaaaaagtagttgaaaaattaagaaaacaatttgggagtaataaaacaatagtcttcaatcactcctcaggaggggacccggaaattgtaatgcacagttttaattgtggaggggaatttttctactgtaatacaacacagttattcaatagtacttggtatcggaatggtacttggtattggaaaggtactaggattgagaatggtactgaagggttaaatgacacaattatcactctcccatgcagaataaaacaaattataaacatgtggcaggaagtaggaaaagcaatgtatgccccgcccatcagcggacaaattagatgttcgtcaaatattacagggctgctactaacaagagatggtggtgagaataatggaactgagaataacgaaactgagactttcagacctggaggaggagatatgaaggacaattggagaagtgaattatataaatataaagtggtaaaaattgaaccattaggagtagcacccaccaaggcaaagagaagagtggtgcagagagaaaaaaga

SEQ ID NO: 70 - DNA, 1476 bp, Artificial Sequence
  ST-199 coding sequence
agtgctgaagaaaacttgtgggtcacagtctattatggggtacctgtgtggagagaagcaaccaccactctattttgtgcatcaggtgctaaagcatatgatacagaggtacataatgtttgggccacacatgcctgtgtacccacagaccccaacccacaagaagtagtattggaaaatgtgacagaaaattttaacatgtggaaaaataacatggtagatcagatgcatgaggatataatcagtttatgggatcaaagcctgaagccatgtgtaaaattaaccccactctgtgttactttagattgcattgatgtggggaatgctactaatggggggaatactactaacaccactagtagtagcggggtaagaatggagccaggagaaataaaaaactgctctttcaatatcaccacaagtataagagataagatgcagaaagaatatgcacttctttataaacttgatttagtaccaatagacaatggtaatagtaatggtagtaatactaccaactataggttgataagttgtaacacctcagtcatcacacaagcctgtcccaaggtaacctttgagccaattcccatacattattgtgctccggctggttttgcgattctaaagtgtaataataagacgttcaatggaacaggaccatgtagaaatgtcagcacagtacaatgtacacatggaattaggccagtagtatcaactcaactgctgttaaatggcagtctagcagaagaagaggtagtaattagatctgaaaatttctcggacaatgctaaaactataatagtacggctgaaccaatctgtagaaattaattgtacaagacccaacaacaatacaagaagaagtataaatataggaccagggagagcactttatgcaacaggagacataataggagatataagacaagcacattgtaatcttagccttgtacaatggaacaacactttaagacaggtagctataaaattaagagaacaatttaagaataaaacaatagtctttaaaaactccacaggaggggacccagagattgtactgcacagttttaattgtggaggggaatttttctactgtaacacaacaaaactgtttaatagtacttggaatggaagtgagactgaagaagttaatacaactgaccaaaatatcacactcccatgcaggataaaacaatttataaacatgtggcaggaagtgggaaaagcaatgtatgccccgtccatcagcggacagatcagatgttcgtcaaatattacagggctgttattaacaagagatggtggtgagaataatggaactgagaataacgaaactgagactttcagacctggaggaggagatatgagggacaattggagaagtgaattatataaatataaagtggtaaaaattgaaccattaggagtagcacccaccagggcaaagagaagagtggtgcagagagaaaaaaga

SEQ ID NO: 71 - DNA, 1449 bp, Artificial Sequence
  ST-272 coding sequence
agtgccgtagaacaattgtgggtcacagtctattatggggtacctgtgtggaaagaagcaaccaccactctattttgtgcatcagacgctaaagcacatgaaacagaagtacataatgtctgggccacacatgcctgtgtacccacagaccccaacccacaagaagtgttattggaaaatgtaacagaaaattttaacatgtggaaaaatagcatggtagatcagatgcatgaggatataatcagcttatgggatcaaagcttaaaaccatgtacaaaactaaccccactctgtgttactttaaattgcactgatgatttgaaaaattttactattaataccaatggaagtatctggcaaaatatggagaaaggagaaataaaaaactgctctttcaatatcaccacaaccttgaaagataaggtgcagagagaatatgcactgttttataaacttgatgtaataccaatagaaaataataatagcagttataggttgatgagttgtaacacctcaaaaattacccaggcttgtccaaaggtatcctttgaaccaattcccatacattattgtaccccggctggttttgcgattctaaagtgtaacgataagaagttcaatggaacaggaccatgtaaaaatgtcagcacagtacagtgtacacatggaatcaggccagtagtgtcaactcaactgctgttaaatggcagtctagcagaagaagacatagtaattagatctgaaaatttcacaaacaatgctaaaaccataatagtacagctaaatgaatctgtagtaattaattgtataagacccaataacaatacaaggaaaaggatatctataggaccagggagagtactttatgcaacaggagaaataataggggatataagacaagcacattgtaacattagtagcacacaatggaataacaccttaggacagatagttgaaaaattaagagcacaatttgggaataaaacaataagatttaatcaaccctcaggaggagacccggaaattgtattgcacagttttaattgtggaggggaatttttctactgtaatacatcaaaactgtttaatggtacttggtattggaaaggtactaggattgagaatggtactgaagggttaaatgacacaattatcacactcccatgcagaataaaacaaattataaatatgtggcagaaagtaggaaaagcaatgtatgccccgcccatcagcggacaaattagatgttcatcgaatatcacagggctgctattaacaagagacggtggtagtagtaatgacaacgatactgagatcttcagacctggaggaggagatatgagggacaattggagaagtgaattatataaatacaaagtagtaaaaattgaaccattaggagtagcacccaccaaggcaaagagaagagtggtgcagagagaaaaaaga

SEQ ID NO: 72 - DNA, 1467 bp, Artificial Sequence
  Syn-ST-003 mammalian codon-optimized coding sequence
tccgccgtggagaagctgtgggtgaccatctactatggcgtgcccgtgtggaaggaggccacgaccacgctgttctgcgcctccgacgccaaggcctacgacaccgaagtgcacaacgtctgggccacccacgcctgcgtgcccaccgaccccaacccccaggaagtggtgctgggcaacgtcaccgagaacttcaatatctggaaaaataacatggtcgagcagatgcacggcgacatcatctccctgtgggatcagtccctcaagccgtgcgtgaagctcacccccctgtgcgtcacgctggactgtatcgacgtcgggaatgccaccaacggcggcaacaccaccaacacgaccggctccggcggggtgcgcatggaacccggcgaaatcaagaattgcagcttcaacattaccaccagcatccgggacaagatgcagaaagaatacgccctgctgtacaaactcgacctggtccccattgacaacgggaactccaacggcagcaataccacgaactaccgcctcatctcctgtaacacgagcgtgattacgcaggcctgtcccaaggtgagcttcgagcccatcccgatccactactgcacccccgccgggtttgcgatcctcaaatgtaacaataagaaattcaacggcaccggcccctgcaagaacgtcagcaccgtccagtgcacgcatggcatccggcccgtggtgtccacccagctgctgctgaatgggagcctcgccgaggaggaggtggtgatccggtccgagaatttctccgacaacgccaagaccatcattgtccagctcaacgagtccgtggtgatcaactgcacccgcccgaacaataatacgcgccgccgcctgagcatcgggccggggcgggtgtggtacaccacgggggagattatcggcgatatccggcaggcgcattgtaacatcagccgcgcgaagtggaacaataccctgcagcagatcgtcatcaagctgcgggagaagttccgcaacaagacgatcgcgttcaatcagagcctcggcggcgatcccgagatcgtgatgcactccttcaactgtggcggcgagttcttttactgcaacaccgcgcagctgttcaactccacgtggaacgacacgaagcgcagcaacatcgagggcaccatcaccctgccctgccggattaagcagatcgtcaacatgtggcaggaggtggggaaggccatgtacgccccccccatcaagggccagatctcctgcagcagcaatatcaccgggctcctgctgacccgggacggcggggagaataacgggacggagaacaatgagaccgagaccttccgccccggcgggggcgacatgcgcgacaactggcgcagcgagctgtataagtacaaggtggtgaagatcgagccgctgggcgtggccccgacccgcgccaagcggcgcgtggtccagcgggagaagcgc

SEQ ID NO: 73 - DNA, 1476 bp, Artificial Sequence
  Syn-ST-057 mammalian codon-optimized coding sequence
agcgtggtggagcagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggaggtggtgctgaagaacgtgaccgagaacttcaacatgtgggacaacaacatggtggaccagatgaacgaggacatcatcagcctgtgggaccagagcctgaagccctgcgtgaagctgacccccctgtgcgtgaccctggactgcatcgacgtgggcaacgccaccaacggcggcaacaccaccaacaccaccagcagcagcggcgtgcgcatggagcccggcgagatcaagaactgcagcttcaacatcaccaccagcatccgcgacaagatgcagaaggagtacgccctgctgtacaagctggacctggtgcccatcgacaacggcaacagcaacggcagcaacaccaccaactaccgcctgatcagctgcaacaccagcgtgatcacccaggcctgccccaaggtgaccttcgagcccatccccatccactactgcgcccccgctggcttcgccatcctgaagtgcaacgacaagaagttcaacggcagcgagccctgccgcaacgtgagcaccgtgcagtgcacccacggcatccgccccgtggtgagcacccagctgctgctgaacggcagcctggccgaggaggaggtggtgatccgcagcgagaacttcaccaacaacgccaagaccatcatcatccagctgaaggagagcgtgaagatcaactgcacccgccccaacaacaacacccgcaagcgcatcagcatcggccccggccgcgccctgtacgccaccggcgacatcatcggcgacatccgccaggcccactgcaacatcagcgagacccagtggaacaacaccctgaagcagatcgtgaagaagctgcgcgagcagttcaagaacaagaccatcgccttcaaccacagcagcggcggcgaccccgagatcgtgatgcacagcttcaactgcggcggcgagttcttctactgcaacaccagcaagctgttcaacagcacctggaacggcagcgagaccgaggaggtgaacaccaccgaccagaacatcaccctgccctgccgcatcaagcagatcgtgaacatgtggcaggaggtgggcaaggccatgtacgccccccccatcagcggccagatccgctgcagcagcaacatcaccggcctgctgctgacccgcgacggcggcgagaacaacggcaccgagaacaacgagaccgagaccttccgccccggcggcggcgacatgcgcgacaactggcgcagcgagctgtacaagtacaaggtggtgaagatcgagcccctgggcgtggcccccaccaaggccaagcgccgcgtggtgcagcgcaagaagcgc

SEQ ID NO: 74 - DNA, 1467 bp, Artificial Sequence
  Syn-ST-128 mammalian codon-optimized coding sequence
agcgccgccaaggagaagacctgggtgaccatctactatggcgtgcccgtgtggaaggaggccacgaccacgctgttctgcgcctccgacgccaaggcctacaagaccgaagtgcacaacgtctgggccacccacacctgcgtgcccaccgaccccaacccccaggaagtggtgctgggcaacgtcaccgagaacttcaatatgtgggacaataacatggtcgaccagatgaacgaagacatcatctccctgtgggatcagtccctcaagccgtgcgtgaagctcacccccctgtgcgtcacgctgaactgtaaggacgtcaacgccaccaacacgacctcctcctccgaggggatgatggaacgcggcgaaatcaagaattgcagcttcaacattaccaagagcatccgggacaaggtgcagaaagaatacgccctgttctacaaactcgacgtcgtccccattgacaacaagaacaacacgaagtaccgcctcatctcctgtaacacgagcgtgattacgcaggcctgtcccaaggtgagcttcgagcccatcccgatccactactgcgcccccgccgggtttgcgatcctcaaatgtaacaataagaccttcaacggcaagggccagtgcaagaacgtcagcaccgtccagtgcacgcatggcatccggcccgtggtgtccacccagctgctgctgaatgggagcctcgccgaggagaaggtggtgatccggtccgacaatttcaccgacaacgccaagaccatcattgtccagctcaacgagtccgtgaaaatcaactgcacccgcccgagcaataatacgcgcaagagcattcacatcgggccggggcgggccttttacaccacgggggagattatcggcgatatccggcaggcgcattgtaacatcagccgcgcgcagtggaacaataccctgaagcagatcgtcgagaagctgcgggagcagttcaacaacaagacgatcgtgttcacccacagcagcggcggcgatcccgagatcgtgatgcactccttcaactgtggcggcgagttcttttactgcaacacctccaagctgttcaactccacgtgggaattcaacagcacctggaacgggaccgagggcctggatgagaccaacaacggcacgggcgggaacatcaccctgccctgccggattaagcagatcgtcaacatgtggcagggggtggggaaggccatgtacgccccccccatcagcggccagatccgctgcagcagcaatatcaccgggctcctgctgacccgggacggcggggagaataacgggacggagaacaatgagaccgagaccttccgccccggcgggggcgacatgcgcgacaactggcgcagcgagctgtataagtacaaggtggtgaagatcgagccgctgggcgtggccccgaccaaggccaagcggcgcgtggtccagcgggagaagcgc

SEQ ID NO: 75 - DNA, 1461 bp, Artificial Sequence
  Syn-ST-168 mammalian codon-optimized coding sequence
tccgccgaggagaacctgtgggtgaccgtgtactatggcgtgcccgtgtggaaggatgccatcaccacgctgttctgcgcctccgacgccaaggcctacgaccgcgaagtgcacaacgtctgggccacccacgcctgcgtgcccaccgaccccaacccccaggaagtggtgctggagaacgtcaccgaggacttcaatatgtggaaaaataacatggtcgagcagatgcaagaagacgtgatcaacctgtgggatcagtccctcaagccgtgcgtgaagctcacccccctgtgcgtcacgctgaactgtatcgacgtcgggaatgccaccaacggcggcaacaccaccaacacgacctcctcctccggggtgcgcatggaacccggcgaaatcaagaattgcagcttcaacattaccaccagcatccgggacaagatgcagaaagaatacgccctgctgtacaaactcgacctggtccccattgacaacgggaactccaacggcagcaataccacgaactaccgcctcatctcctgtaacacgagcgtgattacgcaggcctgtcccaaggtgaccttcgagcccatcccgatccactactgcgcccccgccgggtttgcgatcctcaaatgtaacaataagaccttcaacggcaccgaaccctgccgcaacgtcagcaccgtccagtgcacgcatggcatcaagcccgtggtgtccacccagctgctgctgaatgggagcctcaccgaggacgagatcgtgatccggtccaagaatttctccgacaacgccaagatcatcattgtccagctcaacaagtccgtggagatcaactgcacccgcccgaacaataatacgcgcaagagcgtgagcctcgggccggggtccgcctggtacgccacgggggacattatcggcgatatccggcaggcgcattgtaacctgagcctggtgcagtggaacaataccctgcggcaggtcgccatcaagctgcgggagcagttcaagaacaagacgatcgtgttcaagaacagcaccggcggcgatcccgagatcgtgctgcactccttcaactgtggcggcgagttcttttactgcaacaccaccaagctgttcaactccacgtggaacggcagcgagaccgaggaggtcaacaccaccgatcaaaacatcaccctgccctgccggattaagcagttcatcaacatgtggcagaccgtggggaaggccatgtacgccccccccatcaagggccagatccgctgcagcagcaagatcagcgggctcctgctgacccgggacggcgggaacacgaataacaccgagatcttccgccccggcgggggcgacatgaaagacaactggcgcagcgagctgtataagtacaaggtggtgaagatcgagccgctgggcattgccccgaacaaggccaagcggcgcgtggtccagcgggagcggcgc

SEQ ID NO: 76 - DNA, 1446 bp, Artificial Sequence
  Syn-ST-173 mammalian codon-optimized coding sequence
tccgccaacgagcaaacctgggtgaccgtgtactatggcgtgcccgtgtggaaggagaccacgaccacgctgttctgcgcctccgacgccaaggcctacgacaccgaagtgcacaacgtctgggccacccacgcctgcatccccaccgaccccaacccccaggaagtggtgctgaaaaacgtcaccgagaacttcaatatgtgggacaataacatggtcgagcagatgcaagaagacgtgatcaacctgtgggatcagtccctcaagccgtgcgtgaagctcacccccctgtgcgtcacgctgaactgtaaggacgtcaacgccaccaacacgacctcctcctccgaggggatgatggaacgcggcgaaatcaagaattgcagcttcaacattaccaccagcatccgggacaagatgcagaaagaatacgccctgctgtacaaactcgacctggtccccattgacaacgggaactccaacggcagcaataccacgaactaccgcctcatctcctgtaacacgagcgtgattacgcaggcctgtcccaaggtgaccttcgagcccatcccgatccactactgcgcccccgccgggtttgcgatcctcaaatgtaacgacaagaaattcaacggcaccggcccctgcaccaacgtcagcaccgtccagtgcacgcatggcatccggcccgtggtgtccacccagctgctgctggacgggagcctcgccgaggaggaggtggtgatccggtccgagaatttctccgacaacgccaagaccatcattgtccagctcaaccagtccgtggagatcaactgcacccgcccgaacaataatacgcgccgcagcaccaacatcgggccggagcgggccctgtacgccacgggggagattatcggcgatatccggcaggcgcattgtaacatcagccgcgcgcagtggaacaataccctgcggcaggtcgtcgagaagctgcgggcccagttcgggaacaagacgatccgcttcaatcagcccagcggcggcgatcccgagatcgtgctgcactccttcaactgtggcggcgagttcttttactgcaacacctccaagctgttcaactccacgtggaacgacacgaagcgcagcaacatcgagggcaccatcaccctgccctgccggattaagcagatcgtcaacatgtggcaggaggtggggaaggccatgtacgccccccccatcagcggccagatccgctgcagcagcaatatcaccgggctcctgctgacccgggacggcggggagaataacgggacggagaacaatgagaccgagaccttccgccccggcgggggcgacatgcgcgacaactggcgcagcgagctgtataagtacaaggtggtgaagatcgagccgctgggcgtggccccgacccgcgccaagcggcgcgtggtccagcgggagaagcgc

SEQ ID NO: 77 - DNA, 1482 bp, Artificial Sequence
  Syn-ST-194 mammalian codon-optimized coding sequence
agcgccgccaaggagaagacctgggtgaccatctactatggcgtgcccgtgtggaaggatgccatcaccacgctgttctgcgcctccgacgccaaggcctacgacgccgaagcccacaacgtctgggccacccacgcctgcgtgcccaccgaccccaacccccaggaagtggtgctgggcaacgtcaccgagaacttcaatatgtggaaaaataacatggtcgaccagatgcacgaagacatcatctccctgtgggatcagtccctcaagccgtgcgtgaagctcacccccctgtgcgtcacgctgaactgtaccaattacgtgaagattgacaccaacgccaccgtgtacaattgggagaagatggaagagggcgaaatcaagaattgcagcttcaacgtgaccaccgcgatccgggacaaggtgcagaaagaatacgccctgttctacaaactcgacgtcgtccccattgaggagaacggcaacacgtcctaccgcctcatcaactgtaacacgagcatcattacgcaggcctgtcccaaggtgaccttcgagcccatcccgatccactactgcgcccccgccgggtttgcgatcctcaaatgtaacaataagaccttcaacggcaagggcccctgcaccaacgtcagcaccgtccagtgcacgcatggcatccggcccgtggtgtccacccagctgctgctgaatgggagcctcgccgagaaggaggtgatcctgcggtccgagaatttcacctccaacgccaagaccatcattgtccagctcaacgagaccattaaaatcaactgcacccgcccgaacaataatacgcgcaagggcattcacatcgggccggggcgggccttttacaccacgggggaggtgatcggcaacatccggcaggcgcattgtaacatcagcagcaccgattgggggaagaccctggagaaggtcgtcgagaagctgcggaagcagttcgggtccaacaagacgatcgtgttcaatcacagcagcggcggcgatcccgagatcgtgatgcactccttcaactgtggcggcgagttcttttactgcaacaccacccagctgttcaactccacgtggtaccgcaacggcacctggtactggaaggggacccgcatcgagaacggcaccgagggcctgaacgacacgatcatcaccctgccctgccggattaagcagatcatcaacatgtggcaggaggtggggaaggccatgtacgccccccccatcagcggccagatccgctgcagcagcaatatcaccgggctcctgctgacccgggacggcggggagaataacgggacggagaacaatgagaccgagaccttccgccccggcgggggcgacatgaaagacaactggcgcagcgagctgtataagtacaaggtggtgaagatcgagccgctgggcgtggccccgaccaaggccaagcggcgcgtggtccagcgggagaagcgc

SEQ ID NO: 78 - DNA, 1476 bp, Artificial Sequence
  Syn-ST-199 mammalian codon-optimized coding sequence
tccgccgaggagaacctgtgggtgaccgtgtactatggcgtgcccgtgtggcgcgaggccacgaccacgctgttctgcgcctccggcgccaaggcctacgacaccgaagtgcacaacgtctgggccacccacgcctgcgtgcccaccgaccccaacccccaggaagtggtgctggagaacgtcaccgagaacttcaatatgtggaaaaataacatggtcgaccagatgcacgaagacatcatctccctgtgggatcagtccctcaagccgtgcgtgaagctcacccccctgtgcgtcacgctggactgtatcgacgtcgggaatgccaccaacggcggcaacaccaccaacacgacctcctcctccggggtgcgcatggaacccggcgaaatcaagaattgcagcttcaacattaccaccagcatccgggacaagatgcagaaagaatacgccctgctgtacaaactcgacctggtccccattgacaacgggaactccaacggcagcaataccacgaactaccgcctcatctcctgtaacacgagcgtgattacgcaggcctgtcccaaggtgaccttcgagcccatcccgatccactactgcgcccccgccgggtttgcgatcctcaaatgtaacaataagaccttcaacggcaccggcccctgccgcaacgtcagcaccgtccagtgcacgcatggcatccggcccgtggtgtccacccagctgctgctgaatgggagcctcgccgaggaggaggtggtgatccggtccgagaatttctccgacaacgccaagaccatcattgtccgcctcaaccagtccgtggagatcaactgcacccgcccgaacaataatacgcgccgcagcattaacatcgggccggggcgggccctgtacgccacgggggacattatcggcgatatccggcaggcgcattgtaacctgagcctggtgcagtggaacaataccctgcggcaggtcgccatcaagctgcgggagcagttcaagaacaagacgatcgtgttcaagaacagcaccggcggcgatcccgagatcgtgctgcactccttcaactgtggcggcgagttcttttactgcaacaccaccaagctgttcaactccacgtggaacggcagcgagaccgaggaggtcaacaccaccgatcaaaacatcaccctgccctgccggattaagcagttcatcaacatgtggcaggaggtggggaaggccatgtacgcccccagcatcagcggccagatccgctgcagcagcaatatcaccgggctcctgctgacccgggacggcggggagaataacgggacggagaacaatgagaccgagaccttccgccccggcgggggcgacatgcgcgacaactggcgcagcgagctgtataagtacaaggtggtgaagatcgagccgctgggcgtggccccgacccgcgccaagcggcgcgtggtccagcgggagaagcgc

SEQ ID NO: 79 - DNA, 1449 bp, Artificial Sequence
  Syn-ST-272 mammalian codon-optimized coding sequence
tccgccgtggagcaactgtgggtgaccgtgtactatggcgtgcccgtgtggaaggaggccacgaccacgctgttctgcgcctccgacgccaaggcccacgaaaccgaagtgcacaacgtctgggccacccacgcctgcgtgcccaccgaccccaacccccaggaagtgctcctggagaacgtcaccgagaacttcaatatgtggaaaaatagcatggtcgaccagatgcacgaagacatcatctccctgtgggatcagtccctcaagccgtgcaccaagctcacccccctgtgcgtcacgctgaactgtaccgacgatctgaagaatttcaccatcaacacgaacggctccatctggcagaacatggaaaaaggcgaaatcaagaattgcagcttcaacattaccaccaccctgaaggacaaggtgcagcgcgaatacgccctgttctacaaactcgacgtcatccccattgagaacaacaactcctcctaccgcctcatgtcctgtaacacgagcaagattacgcaggcctgtcccaaggtgagcttcgagcccatcccgatccactactgcacccccgccgggtttgcgatcctcaaatgtaacgacaagaaattcaacggcaccggcccctgcaagaacgtcagcaccgtccagtgcacgcatggcatccggcccgtggtgtccacccagctgctgctgaatgggagcctcgccgaggaggacatcgtgatccggtccgagaatttcaccaacaacgccaagaccatcattgtccagctcaacgagtccgtggtgatcaactgcatccgcccgaacaataatacgcgcaagcgcattagcatcgggccggggcgggtgctgtacgccacgggggagattatcggcgatatccggcaggcgcattgtaacatcagcagcacccagtggaacaataccctgggccagatcgtcgagaagctgcgggcccagttcgggaacaagacgatccgcttcaatcagcccagcggcggcgatcccgagatcgtgctgcactccttcaactgtggcggcgagttcttttactgcaacacctccaagctgttcaacggcacgtggtactggaagggcacccgcatcgagaacggcaccgagggcctgaacgacacgatcatcaccctgccctgccggattaagcagatcatcaacatgtggcagaaggtggggaaggccatgtacgccccccccatcagcggccagatccgctgcagcagcaatatcaccgggctcctgctgacccgggacggcgggtccagcaacgacaatgacaccgagatcttccgccccggcgggggcgacatgcgcgacaactggcgcagcgagctgtataagtacaaggtggtgaagatcgagccgctgggcgtggccccgaccaaggccaagcggcgcgtggtccagcgggagaagcgc

SEQ ID NO: 80 - protein, 476 aa, Human Immunodeficiency Virus type 1
  JRCSF gp120 polypeptide
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKR

SEQ ID NO: 81 - protein, 493 aa, Human Immunodeficiency Virus type 1
  93US073 gp120 polypeptide
SVAEKMWVTVYYGVPVWKEATTTLFCASDAKAYDTEVHNVWATHACVPTDPNPQEVVLENVTENFNMWKSNMVEQMHEDIISLWDQSLKPCVKLTPLCVTLDCIDVGNATNGGNTTNTTSSSGVRMEPGEIKNCSFNITTSIRDKMQKEYALLYKLDLVPIDNGNSNGSNTTNYRLISCNTSVITQACPKVTFEPIPIHYCAPAGFAILKCNNKTFNGTGPCRNVSTVQCTHGIRPVVSTQLLLNGSLAEEEVVIRSENFSDNAKTIIVQLNQSVEINCTRPNNNTRRSINIGPGRALYATGEIIGDIRQAHCNISETQWNNTLKQIVKKLREQFKNKTIAFNHSSGGDPEIVMHSFNCGGEFFYCNTSKLFNSTWNSTWGDTEMNNTEESNNNITLPCRIKQIVNMWQEVGKAMYAPPIRGKIYCRSNITGLLLTRDGGSSNDNDTEIFRPGGGDMRDNWRSELYKYKVVKIEPLGIAPNKAKRRVVQRERR

SEQ ID NO: 82 - protein, 494 aa, Human Immunodeficiency Virus type 1
  92HT593 gp120 polypeptide
SAAENLWVTVYYGVPVWKEATTTLFCASDAKAYETEVHNVWATHACVPTDPNPQEVLLRNVTENFNMWKNNMVEQMHEDIISLWDQSLKPCVKLTPLCVTLECHEVNVNGTANNGTTNVTKNSSNATKSNWGTMEKGEIKNSFNITTNIRDKMQKETAQFYKLDIVPIEDQNKTNNTLYRLINCNTSVITQACPKVSFEPIPIHYCTPAGFAILKCNDRNFNGTGPCKNVSTVQCTHGIKPVVSTQLLLNGSLAEAEVVIRSEDFTNNAKTIIIQLNETVEINCTRPNNNTSKRISIGPGRAFRATKIIGNIRQAHRNISRATWSSTLKKIVAKLREQFGNKTIVFQPSSGGDPEIVMHSFNCGGEFFYCNTTQPFNSTWNSTEESSNTRGKDTITLPCRIKQIINMWQEVGKAMYAPPIRGQIRCSSNITGLLLTRDGGNNNSTNGTEIFRPGGGDMRDNWRSELYKYKVVKIEPLGVAPTKAKRRVVQREKR

SEQ ID NO: 83 - protein, 482 aa, Human Immunodeficiency Virus type 1
  92HT594 gp120 polypeptide
SAVEQLWVTVYYGVPVWKKATTTLFCASDAKAYDREVHNVWATHACVPTDPNPQEVVLENVTENFNMWKNNMVEQMHEDIISLWDQSLKPCVKLTPLCVTLNCTNVKKGTNATNSSEKIIEGGEIKNCSFNVTTSIKDKTTKEHALFYKLDAVPIDSKINKSKNGRYILISCNTSTITQACPKVSFEPIPIHYCAPAGFAILKCNNKTFNGTGPCTNVSTVQCTHGIRPVVSTQLLLNGSLAEEEVVIRSENFTNNAKTIIIQLKESVVINCTRPNNNTRKRISIGPGRVWYTTGQIIGDIRKAHCNLSSTQWNNTLGQIVEKLRAQFGNKTIRFNQPSGGDPEIVLHSFNCGGEFFYCNTSKLFNSTWNDTKRSNIDGTITLPCRIKQIVNMWQEVGKAMYAPPISGQIRCSSNITGLLLTRDGGENNGTENNETETFRPGGGDMRDNWRSELYKYKVVKIEPLGVAPTKAKRRVVQRKKR

SEQ ID NO: 84 - protein, 481 aa, Human Immunodeficiency Virus type 1
  92HT596 gp120 polypeptide
SAVEQLWVTVYYGVPVWKEATTTLFCASDAKAYDREVHDVWATHTCVPTDPNPQEVVLENVTENFNMWKNNMVDQMHEDIISLWDQSLKPCVKLTPLCVTLNCTNVKKGTNATNSSEEMRERGEIKNCSFHVTSSIKDKTTTEHALFYKLDVVPIDARNNSENGSYILISCNTSTITQACPKVSFEPIPIHYCAPAGFAILKCNNKTFNGTGPCTNVSTVQCTHGIRPVVSTQLLLNGSLAEEEVVIRSENFTNNAKTIIIQLKESVVINCTRPNNNTRKRISIGPGRVWYTTGQIIGDIRKAHCNLSSTQWNNTLGQIVEKLRAQFGNKTIRFNQPSGGDPEIVLHSFNCGGEFFYCNTSKLFNSTWNDTKRSNIEGTITLPCRIKQIVNMWQEVGKAMYAPPISGQIRCSSNITGLLLTRDGGENNGTENNETETFRPGGGDMRDNWRSELYKYKVVKIEPLGVAPTRAKRRVVQREKR

SEQ ID NO: 85 - protein, 493 aa, Human Immunodeficiency Virus type 1
  92HT599 gp120 polypeptide
SATERFWVTVYYGVPVWKDAITTLFCASDAKAYDAEAHNVWATHACVPTDPNPQEVVLKNVTENFNMWDNNMVDQMNEDIISLWDQSLKPCVTITPLCVTLHCTDARNSTNVTIEVEKVEKGDIKNCSFNIATNIKGKIQKEYALFSKLDVLPIDNNTGSSNSSRYILISCNTTVVKQACPKVSFEPIPIHYCAPAGFAILKCNDKKFNGSGPCKNVSTVQCTHGIRPAVSTQLLLDGSLAEKEVIIRAENFSNNAKTIIVQLNKAVLINCTRPNNNTRRSVHSGHIGGGRTLFTTHIVGDVRKAHCNLSIAQWNNTLRQVAKKLREQLGDNRTIIFKQSSGGDPEIVTHSFTCGGEFFYCNTSKLFNSTWEFNSTWNGTEGLDETNNGTGGNITLPCRIKQIVNMWQGVGKAMYAPPASGQISCSPKITGLLLTRDGGNVNECKDNETFGPGGGDMKDNWRSELYKYKVVKIEPLGVAPTKAKRRVVQREKR

SEQ ID NO: 86 - protein, 494 aa, Human Immunodeficiency Virus type 1
  92US657 gp120 polypeptide
SAEENLWVTVYYGVPVWKEADTTLFCASDAKAYDTEVHNVWATHACVPTDPNPQEMVSGNVTENFNIWKNNMVEQMHEDIISLWDQSLKPCVKLTPLCVTLNCTNYVKIDTNATVYNWEKMEEGEIKNCSFNVTTAIRDKVQKEYALFYKLDVVPIEENGNTSYRLINCNTSIITQACPKVTFEPIPIHYCAPAGFAILKCNNKTFNGKGPCTNVSTVQCTHGIRPVVSTQLLLNGSLAEKEVILRSENFTSNAKTIIVQLNETIKINCTRPNNNTRKGIHIGPGRAFYTTGEVIGNIRQAHCNISSTDWGKTLEKVVEKLRKQFGSNKTIVFNHSSGGDPEIVMHSFNCGGEFFYCNTTQLFNSTWYRNGTWYWKGTRIENGTEGLNDTIITLPCRIKQIINMWQEVGKAMYAPPIKGQISCSSNITGLLLTRDGGNTNKTGTENETEIFRPGGGDMRDNWRSELYKYKVVKIEPLGVAPTRAKRRVVQREKR

SEQ ID NO: 87 - protein, 478 aa, Human Immunodeficiency Virus type 1
  92US712 gp120 polypeptide
NNAEESWVTVYYGVPVWKEATTTLFCASDAKAYDTEVHNIWATHACVPTDPNPQEVVLENVTENFNMKNNMVKQMHKNIISLWDQSLKPCVKLTPLCVTLNCTDLSNATNTNATTTTNSSAGMMMDRGEIKNCSFNVTASIRNKMQREYALFYKLDVIQIDNTSYRLISCNTSVITQACPKVSFEPIPIHYCAPAGFAILKCNDKKFNGTGPCKNVSTVQCTHGIRPVVSTQLLLNGSLAEEGVVIKSANLSDNAKIIIVQLNESVEMNCIRPNNNTRKSIHIGPGRAFYTTGDIIENIRKAHCNISRAKNNTLKQIAIKLKEQFENKTIVFNQSSGGDPEIMTLMFNCGGEFFYCNSTQLFNSTWNSTGSWNDTGENITLQCRIKQIINMWQEVGKAMYAPPIRGQIRCSSNITGLLLTRDGGINKSENETEIFRPGGGDMRENWRSELYKYKVVKIEPLGVAPTRAKRRVVQREKR

SEQ ID NO: 88 - protein, 482 aa, Human Immunodeficiency Virus type 1
  89.6 gp120 polypeptide
SAAKEKTWVTIYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSSYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKR

SEQ ID NO: 89 - protein, 477 aa, Human Immunodeficiency Virus type 1
  92US727 gp120 polypeptide
SANEQTWVTVYYGVPVWKEATTTLFCASDAKAYKTEVHNVWATHACVPTDPNPQEVRLINVTENFNMWKNNMVEQMHDDIISLWDQSLKPCTKLTPLCVTLNCTDDLKNFTINTNGSIWQNMEKGEIKNCSFNITTTLKDKVQREYALFYKLDVIPIENNNSSYRLMSCNTSKITQACPKVSFEPIPIHYCAPAGFAILKCNDKKFNGSEPCRNVSTVQCTHGIKPVVSTQLLLNGSLTEDEIVIRSKNFSDNAKIIIVQLNKSVEINCTRPNNNTRKSVSLGPGSAWYATGDIIGDIRQAHCNLSLVQWNNTLRQVAIKLREQFKNKTIVFKNSTGGDPEIVLHSFNCGGEFFYCNTTKLFNSTWNGSETEEVNTTDQNITLPCRIKQFINMWQTVGKAMYAPPIKGQIRCSSKISGLLLTRDGGNTNNTEIFRPEGGNMRDNWRSELYKYKVVKIEPLGVAPTKAKRRVVQREKR

SEQ ID NO: 90 - protein, 312 aa, Human Immunodeficiency Virus type 1
  JRCSF gp120 core polypeptide
EVVLENVTEDFNMWKNNMVEQMQEDVINLWDQSLKPCVKLTPLCVGAGSCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIE

SEQ ID NO: 91 - protein, 317 aa, Human Immunodeficiency Virus type 1
  93US073 gp120 core polypeptide
EVVLENVTENFNMWKSNMVEQMHEDIISLWDQSLKPCVKLTPLCVGAGSCNTSVITQACPKVTFEPIPIHYCAPAGFAILKCNNKTFNGTGPCRNVSTVQCTHGIRPVVSTQLLLNGSLAEEEVVIRSENFSDNAKTIIVQLNQSVEINCTGAGHCNISETQWNNTLKQIVKKLREQFKNKTIAFNHSSGGDPEIVMHSFNCGGEFFYCNTSKLFNSTWNSTWGDTEMNNTEESNNNITLPCRIKQIVNMWQEVGKAMYAPPIRGKIYCRSNITGLLLTRNGGSSNDNDTEIFRPGGGDMRDNWRSELYKYKVVKIE

SEQ ID NO: 92 - protein, 314 aa, Human Immunodeficiency Virus type 1
  92HT593 gp120 core polypeptide
EVLLENVTENFNMWKNNMVEQMHEDIISLWDQSLKPCVKLTPLCVGAGICNTSVITQACPKVSFEPIPIHYCTPAGFAILKCNDRNFNGTGPCKNVSTVQCTHGIKPVVSTQLLLNGSLAEAEVVIRSEDFTNNAKTIIIQLNETVEINSTGAGHCNISRATWSSTLKKIVAKLREQFGNKTIVFQPSSGGDPEIVMHSFNCGGEFFYCNTTQPFNSTWNSTEESSNTRGKDTITLPCRIKQIINMWQEVGKAMYAPPIRGQIRCSSNITGLLLTRDGGNNNSTNGTEIFRPGGGDMRDNWRSELYKYKVVKIE

SEQ ID NO: 93 - protein, 313 aa, Human Immunodeficiency Virus type 1
  92HT594 gp120 core polypeptide
EVVLENVTENFNMWKNNMVEQMHEDIISLWDQSLKPCVKLTPLCVGAGSCNTSTITQACPKVSFEPIPIHYCAPAGFAILKCNNKTFNGTGPCTNVSTVQCTHGIRPVVSTQLLLNGSLAEEEVVIRSENFTNNAKTIIIQLKESVVINCTGAGHCNLSSTQWNNTLGQIVEKLRAQFGNKTIRFNQPSGGDPEIVLHSFNCGGEFFYCNTSKLFNSTWNDTKRSNIDGTITLPCRIKQIVNMWQEVGKAMYAPPISGQIRCSSNITGLLLTRDGGENNGTENNETETFRPGGGDMRDNWRSELYKYKVVKIE

SEQ ID NO: 94 - protein, 313 aa, Human Immunodeficiency Virus type 1
  92HT596 gp120 core polypeptide
EVVLGNVTENFNMWKNNMVDQMHEDIISLWDQSLKPCVKLAPLCVGAGSCNTSTITQACPKVSFEPIPIHYCAPAGFAILKCNNKTFNGTGPCTNVSTVQCTHGIRPVVSTQLLLNGSLAEEEVVIRSENFTNNAKTIIIQLKESVVINCTGAGHCNLSSTQWNNTLGQIVEKLRAQFGNKTIRFNQPSGGDPEIVLHSFNCGGEFFYCNTSKLFNSTWNDTKRSNIEGTITLPCRIKQIVNMWQEVGKAMYAPPISGQIRCSSNITGLLLTRDGGENNGTENNETETFRPGGGDMRDNWRSELYKYKVVKIE

SEQ ID NO: 95 - protein, 323 aa, Human Immunodeficiency Virus type 1
  92HT599 gp120 core polypeptide
EVVLKNVTENFNMWDNNMVDQMNEDIISLWDQSLKPCVTITPLCVGAGSCNTTVVKQACPKVSFEPIPIHYCAPAGFAILKCNDKKFNGSGPCKNVSTVQCTHGIRPAVSTQLLLDGSLAEKEVIIRAENFSNNAKTIIVQLNKAVLINCTGAGHCNLSIAQWNNTLRQVAKKLREQLGDNRTIIFKQSSGGDPEIVTHSFTCGGEFFYCNTSKLFNSTWEFNSTWNGTEGLDETNNGTGGNITLPCRIKQIVNMWQGVGKAMYAPPASGQISCSPKITGLLLTRDGGNVNECKDNETFGPGGGDMKDNWRSELYKYKVVKIE

SEQ ID NO: 96 - protein, 328 aa, Human Immunodeficiency Virus type 1
  92US657 gp120 core polypeptide
EMVLGNVTENFNIWKNNMVEQMHEDIISLWDQSLKPCVKLTPLCVGAGNCNTSIITQACPKVTFEPIPIHYCAPAGFAILKCNNKTFNGKGPCTNVSTVQCTHGIRPVVSTQLLLNGSLAEKEVILRSENFTSNAKTIIVQLNETIKINCTGAGHCNISSTDWGKTLEKVVEKLRKQFGSNKTIVFNHSSGGDPEIVMHSFNCGGEFFYCNTTQLFNSTWYRNGTWYWKGTRIENGTEGLNDTIITLPCRIKQIINMWQEVGKAMYAPPIKGQISCSSNITGLLLTRDGGNTNKTGTENETEIFRPGGGDMRDNWRSELYKYKVVKIE

SEQ ID NO: 97 - protein, 310 aa, Human Immunodeficiency Virus type 1
  92US712 gp120 core polypeptide
EVVLENVTENFNMKNNMVKQMHKNIISLWDQSLKPCVKLTPLCVGAGSCNTSVITQACPKVSFEPIPIHYCAPAGFAILKCNDKKFNGTGPCKNVSTVQCTHGIRPVVSTQLLLNGSLAEEGVVIKSANLSDNAKIIIVQLNESVEMNCIGAGHCNISRAKNNTLKQIAIKLKEQFENKTIVFNQSSGGDPEIMTLMFNCGGEFFYCNSTQLFNSTWNSTGSWNDTGENITLQCRIKQIINMWQEVGKAMYAPPIRGQIRCSSNITGLLLTRDGGINKSENETEIFRPGGGDMRENWRSELYKYKVVKIE

SEQ ID NO: 98 - protein, 313 aa, Human Immunodeficiency Virus type 1
  89.6 gp120 core polypeptide
EVVLGNVTENFNMWKNNMVDQMHEDIISLWDESLKPCVKLTPLCVGAGSCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTGAGHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIE

SEQ ID NO: 99 - protein, 12 aa, Artificial Sequence
  Polyhistidine / GS linker
SGSHHHHHHGGS

SEQ ID NO: 100 - protein, 340 aa, Artificial Sequence
  JRCSF Core+V3
EVVLENVTEDFNMWKNNMVEQMQEDVINLWDQSLKPCVKLTPLCVGAGSCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIE

SEQ ID NO: 101 - protein, 312 aa, Artificial Sequence
  JRCSF Core(V4-GS2)
EVVLENVTEDFNMWKNNMVEQMQEDVINLWDQSLKPCVKLTPLCVGAGSCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGGSGNDTIILPCRIKQIINMWQEVGKAMYAPPIKGQIRCSSNITGLLLTRDGGKNESEIEIFRPGGGDMRDNWRSELYKYKVVKIE

SEQ ID NO: 102 - protein, 312 aa, Artificial Sequence
  JRCSF Core(V4-GS8)
EVVLENVTEDFNMWKNNMVEQMQEDVINLWDQSLKPCVKLTPLCVGAGSCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTGAGHCNISRAQWNNTLKQIVEKLREQFNNKTIVFTHSSGGDPEIVMHSFNCGGEFFYCNSTQLFNSTWNGSGGSSGGSGGSTIILPCRIKQIINMWQEVGKAMYAPPIKGQIRCSSNITGLLLTRDGGKNESEIEIFRPGGGDMRDNWRSELYKYKVVKIE

SEQ ID NO: 103 - protein, 312 aa, Artificial Sequence
  JRCSF Core(V5-GS3)
EVVLENVTEDFNMWKNNMVEQMQEDVINLWDQSLKPCVKLTPLCVGAGSCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGSGGSEIEIFRPGGGDMRDNWRSELYKYKVVKIE

SEQ ID NO: 104 - protein, 448 aa, Artificial Sequence
  JRCSF gp120(d)V3
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKR

SEQ ID NO: 105 - protein, 385 aa, Artificial Sequence
  JRCSF gp120(d)V1V2V3
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVGAGSCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKR

SEQ ID NO: 106 - protein, 375 aa, Artificial Sequence
  JRCSF Core+V1V2
EVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIE

SEQ ID NO: 107 - protein, 460 aa, Artificial Sequence
  ST-008 gp120(d)V3
GAAEQLWVTVYFGVPVWEEATTTLFCASDAKAYDTEVHNIWATHACVPTDPNPQEVVLENVTENFNMWKNNMVDQMHEDIISLWDQSLKPCIKLIPLCVTLDCIDVGNATNGGDTTNTTSSSGVRMEPGEIKNCSFNITTSIRDKMQKEYALLYKLDLVPIDNGNSNGSNTTNYRLISCNTSVITQACPKVTFEPIPIHYCAPAGFAILKCNNKTFNGTGPCRNVSTVQCTHGIRPVVSTQLLLNGSLAEEEVVIRSENFSDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKR

SEQ ID NO: 108 - protein, 385 aa, Artificial Sequence
  ST-008 gp120(d)V1V2V3
GAAEQLWVTVYFGVPVWEEATTTLFCASDAKAYDTEVHNIWATHACVPTDPNPQEVVLENVTENFNMWKNNMVDQMHEDIISLWDQSLKPCIKLIPLCVGAGSCNTSVITQACPKVTFEPIPIHYCAPAGFAILKCNNKTFNGTGPCRNVSTVQCTHGIRPVVSTQLLLNGSLAEEEVVIRSENFSDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKR

SEQ ID NO: 109 - protein, 312 aa, Artificial Sequence
  ST-008 Core
EVVLENVTENFNMWKNNMVDQMHEDIISLWDQSLKPCIKLIPLCVGAGSCNTSVITQACPKVTFEPIPIHYCAPAGFAILKCNNKTFNGTGPCRNVSTVQCTHGIRPVVSTQLLLNGSLAEEEVVIRSENFSDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIE

SEQ ID NO: 110 - protein, 387 aa, Artificial Sequence
  ST-008 Core+V1V2
EVVLENVTENFNMWKNNMVDQMHEDIISLWDQSLKPCIKLIPLCVTLDCIDVGNATNGGDTTNTTSSSGVRMEPGEIKNCSFNITTSIRDKMQKEYALLYKLDLVPIDNGNSNGSNTTNYRLISCNTSVITQACPKVTFEPIPIHYCAPAGFAILKCNNKTFNGTGPCRNVSTVQCTHGIRPVVSTQLLLNGSLAEEEVVIRSENFSDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIE

SEQ ID NO: 111 - protein, 648 aa, Artificial Sequence
  gp140 JRCSF(all)
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 112 - protein, 648 aa, Artificial Sequence
  gp140 JRCSF(null)
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKQCSFNITKSIRDKVQKEYALFYKLDVVPIDNKQNTKYRLISCQTSVITQACPKVSFEPIPIHYCAPAGFAILKCNNKTFNGKGQCKNVSTVQCTHGIRPVVSTQLLLNGSLAEEKVVIRSDQFTDNAKTIIVQLNESVKIQCTRPSQNTRKSIHIGPGRAFYTTGEIIGDIRQAHCQISRAQWNNTLKQIVEKLREQFNNKTIVFTHSSGGDPEIVMHSFNCGGEFFYCQSTQLFNSTWNDTEKSSGTEGNDTIILPCRIKQIINMWQEVGKAMYAPPIKGQIRCSSQITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 113 - protein, 648 aa, Artificial Sequence
  gp140 L16B-001
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKQNTKYRLISCNTSVITQACPKVSFEPIPIHYCAPAGFAILKCNNKTFNGKGQCKNVSTVQCTHGIRPVVSTQLLLNGSLAEEKVVIRSDQFTDNAKTIIVQLNESVKIQCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSQITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 114 - protein, 648 aa, Artificial Sequence
  gp140 L16B-002
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKQCSFNITKSIRDKVQKEYALFYKLDVVPIDNKQNTKYRLISCQTSVITQACPKVSFEPIPIHYCAPAGFAILKCNNKTFNGKGQCKNVSTVQCTHGIRPVVSTQLLLNGSLAEEKVVIRSDQFTDNAKTIIVQLNESVKIQCTRPSNNTRKSIHIGPGRAFYTTGEIIGDIRQAHCQISRAQWNNTLKQIVEKLREQFNNKTIVFTHSSGGDPEIVMHSFNCGGEFFYCQSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 115 - protein, 648 aa, Artificial Sequence
  gp140 L16B-003
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKQNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCQISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 116 - protein, 648 aa, Artificial Sequence
  gp140 L16B-004
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKQNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKIQCTRPSQNTRKSIHIGPGRAFYTTGEIIGDIRQAHCNISRAQWNNTLKQIVEKLREQFNNKTIVFTHSSGGDPEIVMHSFNCGGEFFYCQSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 117 - protein, 648 aa, Artificial Sequence
  gp140 L16B-005
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKQNTKYRLISCNTSVITQACPKVSFEPIPIHYCAPAGFAILKCNNKTFNGKGQCKNVSTVQCTHGIRPVVSTQLLLNGSLAEEKVVIRSDQFTDNAKTIIVQLNESVKIQCTRPSQNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSQITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 118 - protein, 648 aa, Artificial Sequence
  gp140 L16B-006
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDQFTDNAKTIIVQLNESVKIQCTRPSQNTRKSIHIGPGRAFYTTGEIIGDIRQAHCQISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 119 - protein, 648 aa, Artificial Sequence
  gp140 L16B-007
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKQCSFNITKSIRDKVQKEYALFYKLDVVPIDNKQNTKYRLISCQTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 120 - protein, 648 aa, Artificial Sequence
  gp140 L16B-008
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKQNTKYRLISCQTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSQNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 121 - protein, 648 aa, Artificial Sequence
  gp140 L16B-009
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDQFTDNAKTIIVQLNESVKINCTRPSNNTRKSIHIGPGRAFYTTGEIIGDIRQAHCQISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSQITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 122 - protein, 648 aa, Artificial Sequence
  gp140 L16B-010
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKQCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDQFTDNAKTIIVQLNESVKIQCTRPSQNTRKSIHIGPGRAFYTTGEIIGDIRQAHCNISRAQWNNTLKQIVEKLREQFNNKTIVFTHSSGGDPEIVMHSFNCGGEFFYCQSTQLFNSTWNDTEKSSGTEGNDTIILPCRIKQIINMWQEVGKAMYAPPIKGQIRCSSQITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 123 - protein, 648 aa, Artificial Sequence
  gp140 L16B-024
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKIQCTRPSQNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 124 - protein, 648 aa, Artificial Sequence
  gp140 L16B-199
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 125 - protein, 648 aa, Artificial Sequence
  gp140 L16B-217
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKQNTKYRLISCQTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKIQCTRPSQNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFGITKWLWYIK

SEQ ID NO: 126 - protein, 476 aa, Artificial Sequence
  JRCSF gp120 pMV-0612
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKIQCTRPSQNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKR

SEQ ID NO: 127 - protein, 476 aa, Artificial Sequence
  JRCSF gp120 pMV-0613
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPSNNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKR

SEQ ID NO: 128 - protein, 476 aa, Artificial Sequence
  JRCSF gp120 pMV-0614
SAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRDKVQKEYALFYKLDVVPIDNKQNTKYRLISCQTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKIQCTRPSQNTRKSIHIGPGRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIEPLGVAPTKAKRRVVQREKR

SEQ ID NO: 129 - protein, 312 aa, Artificial Sequence
  JRCSF core pMV-0780
EVVLENVTEDFNMWKNNMVEQMQEDVINLWDQSLKPCVKLTPLCVGAGSCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTGAGHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKQESEIEIFRPGGGDMRDNWRSELYKYKVVKIE

SEQ ID NO: 130 - protein, 341 aa, Artificial Sequence
  92HT594 Core+V3
EVVLENVTENFNMWKNNMVEQMHEDIISLWDQSLKPCVKLTPLCVGAGSCNTSTITQACPKVSFEPIPIHYCAPAGFAILKCNNKTFNGTGPCTNVSTVQCTHGIRPVVSTQLLLNGSLAEEEVVIRSENFTNNAKTIIIQLKESVVINCTRPNNNTRKRISIGPGRVWYTTGQIIGDIRKAHCNLSSTQWNNTLGQIVEKLRAQFGNKTIRFNQPSGGDPEIVLHSFNCGGEFFYCNTSKLFNSTWNDTKRSNIDGTITLPCRIKQIVNMWQEVGKAMYAPPISGQIRCSSNITGLLLTRDGGENNGTENNETETFRPGGGDMRDNWRSELYKYKVVKIE

SEQ ID NO: 131 - protein, 312 aa, Artificial Sequence
  L7-043_V4V5 JR
EVVLENVAENFNMWKNNMVEQMHEDIIGLWDQSLKPCVKLTPLCVGAGSCNTSTITQARPKVSFEPIPIHYCAPAGFAILKCNDKKFNGTGPCKNVSTVQCTHGIRPVVSTQLLLNGSLAEEEVVIRSENFSDNAKTIIVQLNESVVINCTGAGHCNISSTQWNNTLGQIVEKLRAQFGNKTIRFNQPSGGDPEIVLHSFNCGGEFFYCNSTQLFNSTWNDTEKSSGTEGNDTIILPCRIKQIVNMWQEVGKAMYAPPIKGQIRCSSNITGLLLTRDGGKNESEIEIFRPGGGDMRDNWRSELYKYKVVKIE

SEQ ID NO: 132 - protein, 315 aa, Artificial Sequence
  L7-043_V4 JR
EVVLENVAENFNMWKNNMVEQMHEDIIGLWDQSLKPCVKLTPLCVGAGSCNTSTITQARPKVSFEPIPIHYCAPAGFAILKCNDKKFNGTGPCKNVSTVQCTHGIRPVVSTQLLLNGSLAEEEVVIRSENFSDNAKTIIVQLNESVVINCTGAGHCNISSTQWNNTLGQIVEKLRAQFGNKTIRFNQPSGGDPEIVLHSFNCGGEFFYCNSTQLFNSTWNDTEKSSGTEGNDTIILPCRIKQIVNMWQEVGKAMYAPPIKGQIRCSSNITGLLLTRDGGENNGTENNETETFRPGGDMRDNWRSELYKYKVVKIE

SEQ ID NO: 133 - protein, 309 aa, Artificial Sequence
  L7-043_V5 JR
EVVLENVAENFNMWKNNMVEQMHEDIIGLWDQSLKPCVKLTPLCVGAGSCNTSTITQARPKVSFEPIPIHYCAPAGFAILKCNDKKFNGTGPCKNVSTVQCTHGIRPVVSTQLLLNGSLAEEEVVIRSENFSDNAKTIIVQLNESVVINCTGAGHCNISSTQWNNTLGQIVEKLRAQFGNKTIRFNQPSGGDPEIVLHSFNCGGEFFYCNTSKLFNSTWNDTKRSNIDGTITLPCRIKQIVNMWQEVGKAMYAPPIKGQIRCSSNITGLLLTRDGGKNESEIEIFRPGGGDMRDNWRSELYKYKVVKIE

SEQ ID NO: 134 - protein, 340 aa, Artificial Sequence
  L7-043 Core+V3 JR
EVVLENVAENFNMWKNNMVEQMHEDIIGLWDQSLKPCVKLTPLCVGAGSCNTSTITQARPKVSFEPIPIHYCAPAGFAILKCNDKKFNGTGPCKNVSTVQCTHGIRPVVSTQLLLNGSLAEEEVVIRSENFSDNAKTIIVQLNESVVINCTRPSNNTRKSIHIGPGRAFYTTGEIIGDIRQAHCNISSTQWNNTLGQIVEKLRAQFGNKTIRFNQPSGGDPEIVLHSFNCGGEFFYCNTSKLFNSTWNDTKRSNIDGTITLPCRIKQIVNMWQEVGKAMYAPPIKGQIRCSSNITGLLLTRDGGENNGTENNETETFRPGGDMRDNWRSELYKYKVVKIE

(19) United States
(12) Patent Application Publication
(10) Pub. No.: US 2011/0262474 A1
(43) Pub. Date: Oct. 27, 2011

(54) Chimeric HIV Antigens

(76) Inventors: Xiaohan Du, Fremont, CA (US); Li Xu, Cupertino, CA (US); Robert Whalen, Foster City, CA (US); Kristin M. Ostrow, Redwood City, CA (US)

(21) Appl. No.: 12/670,115

(22) PCT Filed: Jul. 23, 2008
(86) PCT No.: PCT/US2008/070947

§ 371 Date: Sep. 21, 2010

Related U.S. Application Data
(60) Provisional application No. 60/951,396, filed on Jul. 23, 2007.
(60) Provisional application No. 60/953,079, filed on Jul. 31, 2007.
(60) Provisional application No. 60/953,069, filed on Jul. 31, 2007.

(51) Int. Cl. A61K 39/21; C07K 19/00; C07H 21/02; C12N 15/63; C12N 7/01; C12N 7/00; C12N 7/04; C12N 5/10; C12N 1/19; C12N 1/21; C07K 16/10; C12N 5/12; C12P 21/02; A61P 37/04; A61P 31/18; C07K 14/16

(52) U.S. Cl. 424/188.1; 530/350; 530/395; 530/405; 536/23.72; 536/23.4; 424/208.1; 435/320.1; 435/235.1; 435/236; 435/325; 435/419; 435/254.2; 435/252.3; 435/348; 435/362; 435/369; 435/366; 435/254.21; 424/199.1; 530/387.9; 435/331; 435/69.3

(57) ABSTRACT

The invention provides polynucleotides and polypeptides encoded therefrom that are capable of inducing immune responses to a human immunodeficiency virus. Compositions and methods for utilizing polynucleotides and polypeptides of the invention are also provided.

### CROSS-REFERENCE TO RELATED APPLICATIONS

Pursuant to 35 U.S.C. §119(e), this application claims the benefit of U.S. Provisional Application Ser. No. 60/953,069 filed on Jul. 31, 2007, U.S. Provisional Application Ser. No. 60/953,079 filed on Jul. 31, 2007, and U.S. Provisional Application Ser. No. 60/951,396 filed on Jul. 23, 2007, the disclosures of each of which are incorporated by reference herein in their entirety for all purposes.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

This invention pertains generally to polypeptides that induce an immune response against one or more human immunodeficiency viruses, polynucleotides encoding such polypeptides, methods of making and using such polypeptides and polynucleotides, and diagnostic assays employing such polypeptide and polynucleotides.

### BACKGROUND OF THE INVENTION

The human immunodeficiency virus (HIV) is the agent that causes acquired immunodeficiency syndrome (AIDS) in humans. The global AIDS epidemic can likely only be mitigated by the development of a vaccine(s) to prevent the spread of the virus. At the present time, there is no effective prophylactic vaccine that prevents HIV infection or transmission following exposure to the virus. Although some progress in the development of therapeutic treatments for HIV-infected individuals has been reported, the need for a therapeutic or prophylactic HIV vaccine or drug candidate that enhances immune response to HIV still exists. The present invention addresses the need for molecules that induce or enhance immune responses to HIV, including molecules that would be of beneficial use in prophylactic and therapeutic treatment regimens and/or as vaccines.

### SUMMARY OF THE INVENTION

In one aspect, the present invention provides an isolated or recombinant polypeptide comprising a polypeptide sequence having at least 90% sequence identity to a polypeptide sequence selected from the group consisting of SEQ ID NOS:1-21 and 56-63, wherein the polypeptide induces an immune response against at least one human immunodeficiency virus (HIV) or pseudovirus. Some such polypeptides of the invention induce an immune response against at least one human immunodeficiency type 1 (HIV-1) or pseudovirus. Some such polypeptides of the invention each induce an immune response against at least two HIV-1 viruses or pseudoviruses that are of the same HIV-1 virus subtype or of different HIV-1 virus subtypes. The immune response may comprise an anti-HIV antibody response or HIV-specific T cell immune response or both. The anti-HIV antibody response may be an anti-HIV neutralizing response. Some such polypeptides induce the production of antibodies capable of binding to at least one HIV virus or pseudovirus (e.g., HIV-1 virus or pseudovirus).

Some such polypeptides of the invention are capable of inducing an immune response against at least one HIV virus or pseudovirus, such as at least one HIV-1 or HIV-1 pseudovirus, in a subject to whom at least one such polypeptide is administered in an amount effective to induce the immune response. The induced immune response may comprise an immune response against at least two HIV-1 viruses or pseudoviruses that are of the same subtype (i.e., clade) or of different subtypes. The induced immune response may comprise a neutralizing antibody response against one or more HIV viruses or pseudoviruses. Such polypeptides that induce an immune response against at least one HIV virus (e.g., HIV-1) are useful in a prophylactic or therapeutic treatment or as a prophylactic or therapeutic vaccine against HIV infection (e.g., HIV-1). Some such polypeptides are capable of inhibiting or preventing HIV infection in a subject to whom an amount of at least one such polypeptide effective to inhibit or prevent infection by at least one HIV virus (e.g., HIV-1) is administered and thus are useful in a prophylactic or therapeutic treatment or as a prophylactic or therapeutic vaccine against infection by the at least one HIV virus (e.g., HIV-1).

The invention also provides an isolated or recombinant polypeptide comprising a polypeptide sequence having at least 90% sequence identity to a polypeptide sequence selected from the group consisting of SEQ ID NOS:1-21 and 56-63, wherein the polypeptide binds to or reacts with an anti-HIV-1 antibody, such as an HIV-1 neutralizing antibody.

The invention further provides an isolated or recombinant polypeptide comprising a polypeptide sequence having at least 90% sequence identity to a polypeptide sequence selected from the group of SEQ ID NOS:1-21 and 56-63, wherein the polypeptide induces an immune response in a subject that is cross reactive against 2, 3, 4, 5, 6, 7, 8, 9, or 10 different HIV-1 viruses.

Also provided is an isolated or recombinant polypeptide comprising a fragment of a gp120 variant polypeptide sequence, the gp120 variant polypeptide sequence having at least 90% sequence identity to a sequence selected from the group consisting of SEQ ID NOS:1-7 and 56-63, wherein the fragment comprises at least those amino acid residues of the selected polypeptide sequence located at positions corresponding by reference to amino acid residues of regions C2, C3, V4, C4, and V5 of the recombinant HIV-1 gp120-HXB2 envelope protein sequence (SEQ ID NO:54) shown in FIGS. 10A-10F, wherein the amino acid residues of the fragment are numbered by reference to amino acid residues of the gp120-HXB2 envelope protein, wherein the polypeptide induces an immune response against at least one HIV virus or pseudovirus.

The invention also provides an isolated or recombinant polypeptide comprising a first, a second, a third, a fourth and a fifth subsequence of a gp120 variant sequence, the gp120 variant sequence comprising a an amino acid sequence having at least 90% sequence identity to a polypeptide sequence selected from the group consisting of SEQ ID NOS:1-21 and 56-63, wherein: (a) the first subsequence of the gp120 variant sequence comprises a sequence corresponding by reference to amino acid residues 83-127 of the C1 region of the recombinant HXB2 gp120 envelope protein (SEQ ID NO:54) shown in FIGS. 10A-10F, wherein the C-terminus of the first subsequence is covalently linked by a peptide bond to the N-terminus of a first linker peptide; (b) the second subsequence of the gp120 variant sequence corresponds by reference to the C2 region of the recombinant HXB2 gp120 envelope protein (SEQ ID NO:54) shown in FIGS. 10A-10F, wherein the N-terminus of the second subsequence is covalently linked by a peptide bond to the C-terminus of the first linker peptide, and the C-terminus of the second subsequence is covalently linked by a peptide bond to the N-terminus of a second linker peptide or a gp120 V3 region sequence; (c) the third subsequence of the gp120 variant sequence corresponds by reference to the C3 region of the recombinant HXB2 gp120 envelope protein (SEQ ID NO:54) shown in FIGS. 10A-10F, and the N-terminus of the third subsequence is covalently linked by a peptide bond to the C-terminus of the second linker polypeptide or the gp120 V3 region sequence, and the C-terminus of the third subsequence is covalently linked by a peptide bond to the N-terminus of a gp120 V4 region sequence; (d) the fourth subsequence of the gp120 variant sequence corresponds by reference to the C4 region of the recombinant HXB2 gp120 envelope protein (SEQ ID NO:54) shown in FIGS. 10A-10F, and the N-terminus of the fourth subsequence is covalently linked by a peptide bond to the C-terminus of the gp120 V4 region sequence, and the C-terminus of the fourth subsequence is covalently linked by a peptide bond to the N-terminus of a gp120 V5 region sequence; and (e) the fifth subsequence of the gp120 variant sequence comprises a sequence corresponding by reference to amino acid residues 472-492 of the C5 region of the recombinant HXB2 gp120 envelope protein (SEQ ID NO:54) shown in FIGS. 10A-10F, and the N-terminus of the fifth subsequence is covalently linked by a peptide bond to the C-terminus of the V5 region sequence; wherein the gp120 V3 region sequence, the gp120 V4 region sequence and the gp120 V5 region sequence correspond by reference to the V3 region, the V4 region, and the V5 region, respectively, of the recombinant HXB2 gp120 envelope protein (SEQ ID NO:54) shown in FIGS. 10A-10F, and one or more of the gp120 V3 region sequence, the gp120 V4 region sequence and the gp120 V5 region sequence is not a subsequence of the selected gp120 variant sequence; and wherein the polypeptide induces an immune response against at least one HIV virus or pseudovirus.

Also provided is an isolated or recombinant polypeptide comprising a polypeptide sequence having at least 90% sequence identity to a sequence selected from the group consisting of SEQ ID NOS:1-21 and 56-63, wherein the polypeptide sequence comprises an amino acid substitution in a glycosylation motif (N-X-S/T) which eliminates N-linked glycosylation at one or more glycosylation sites selected from N156, N188, N197, N276, N295, N301, N332, N386, N448, and N461, wherein the amino acid residues are numbered according to the amino acid residues of the recombinant gp120-HXB2 envelope protein (SEQ ID NO:54) as shown in FIGS. 10A-10F, wherein the polypeptide induces an immune response against at least one HIV virus or pseudovirus. Some such deglycosylated variants induce an increased immune response against at least one human immunodeficiency virus type 1 (HIV-1 virus) or pseudovirus, compared to the immune response induced by the parent polypeptide (i.e., the polypeptide lacking substitutions at any of the glycosylation sites).

The invention further provides an isolated or recombinant HIV-1 gp120 polypeptide variant comprising a polypeptide sequence that differs from the polypeptide sequence of any of the group consisting of SEQ ID NOS:1-21 and 56-63 by no more than 1, 2, 3, 4, 5, 6, 7, 9, 10, 15, 20, or 25 amino acid residues, wherein the polypeptide variant induces the production of neutralizing antibodies against at least one HIV-1 virus in a subject to whom an effective amount of the variant is administered.

The invention also includes an isolated or recombinant nucleic acid comprising a polynucleotide sequence that encodes any polypeptide of the invention, or a complementary polynucleotide sequence thereof.

The invention further includes an isolated or recombinant nucleic acid that induces an immune response against at least one HIV virus or pseudovirus (e.g., HIV-1) in a subject to whom an effective amount of the nucleic acid is administered, wherein the nucleic acid comprises a polynucleotide sequence having at least 80% sequence identity to at least one nucleic acid sequence selected from the group consisting of SEQ ID NOS:23-50 and 64-79, or a complementary polynucleotide sequence thereof. The invention further includes an isolated or recombinant nucleic acid that induces an immune response against at least one HIV virus or pseudovirus (e.g., HIV-1) in a subject to whom an effective amount of the nucleic acid is administered, wherein the nucleic acid comprises a polynucleotide sequence which encodes a polypeptide sequence having at least 90% amino acid sequence identity to a polypeptide sequence selected from the group consisting of SEQ ID NOS:1-21 and 56-63, or a complementary polynucleotide sequence thereof. The immune response may comprise the production neutralizing antibodies against at least one HIV virus or pseudovirus (e.g., HIV-1) in a subject to whom an effective amount of the nucleic acid is administered.

In another aspect, the invention includes an isolated or recombinant nucleic acid that induces an immune response against HIV-1 in a subject to whom an effective amount of the nucleic acid is administered, wherein said nucleic acid comprises a polynucleotide sequence having at least 80% sequence identity to an RNA polynucleotide sequence, said RNA polynucleotide sequence comprising a DNA sequence selected from the group of SEQ ID NOS:23-50 and 64-79 in which all of the thymine nucleotide residues in said DNA sequence are replaced with uracil nucleotide residues, or a complementary polynucleotide sequence thereof. The invention also includes an isolated or recombinant nucleic acid that induces an immune response against HIV-1 in a subject to whom an effective amount of the nucleic acid is administered, wherein said nucleic acid comprises a polynucleotide sequence having at least 80% sequence identity to an RNA polynucleotide sequence, said RNA polynucleotide sequence comprising a DNA sequence which encodes a polypeptide sequence having at least 90% amino acid sequence identity to a polypeptide sequence selected from the group consisting of SEQ ID NOS:1-21 and 56-63, in which all of the thymine nucleotide residues in said DNA sequence are replaced with uracil nucleotide residues, or a complementary polynucleotide sequence thereof.

In another aspect, the invention provides a vector comprising at least one nucleic acid of the invention, including a nucleic acid that encodes a polypeptide having at least 90% sequence identity to a polypeptide sequence selected from the group consisting of SEQ ID NOS:1-21 and 56-63, wherein the polypeptide expressed by the vector is capable of inducing a neutralizing antibody response against one or more HIV-1 viruses in a host to which an effective amount of the vector is administered.

The invention also provides a virus or virus-like particle (VLP) comprising at least one polypeptide and/or at least one nucleic acid of the invention. Also included is an attenuated or replication-deficient virus or pseudovirus comprising at least one polypeptide and/or at least one nucleic acid of the invention. In another aspect, the invention provides a cell comprising at least one polypeptide, nucleic acid, and/or vector of the invention.

The invention also includes compositions comprising at least one polypeptide, nucleic acid, virus, virus-like particle, pseudovirus, vector, and/or cell such as, e.g., those described above, and a carrier or excipient.

In another aspect, the invention provides a method of inducing an immune response against an HIV virus or pseudovirus (e.g., HIV-1) in a mammalian cell or mammalian host comprising administering to the cells or host, respectively, an effective amount of: 1) at least one nucleic acid of the invention, such as a nucleic acid having at least 90% sequence identity to a nucleotide sequence selected from the group consisting of SEQ ID NOS:23-50 and 64-79, wherein the nucleic acid encodes a polypeptide that induces an immune response (e.g., neutralizing antibodies) in the cells or host against at least one HIV-1 virus or pseudovirus; 2) at least one vector comprising a nucleic acid of the invention, such as a nucleic acid having at least 90% sequence identity to a nucleotide sequence selected from the group consisting of SEQ ID NOS:23-50 and 64-79, wherein the nucleic acid encodes a polypeptide that induces an immune response (e.g., neutralizing antibodies) in the cells or host against at least one HIV-1 virus or pseudovirus; 3) at least one polypeptide of the invention, such as a polypeptide having at least 90% sequence identity to a polypeptide sequence selected from the group consisting of SEQ ID NOS:1-21 and 56-63, wherein the polypeptide that induces an immune response (e.g., neutralizing antibodies) in the cells or host against at least one HIV-1 virus or pseudovirus; and/or 4) at least one virus or virus-like particle comprising a polypeptide of the invention, such as a polypeptide having at least 90% sequence identity to a polypeptide sequence selected from the group consisting of SEQ ID NOS:1-21 and 56-63, wherein said polypeptide induces an immune response against at least one HIV-1 virus or pseudovirus.

The invention also provides a method of inducing an immune response against at least one HIV virus or pseudovirus (e.g., HIV-1) in a subject, comprising administering to the subject an effective amount of at least one polypeptide, nucleic acid, vector, virus, virus-like particle, pseudovirus, cell or population of cells of the invention, or any combination thereof of any of the foregoing, sufficient to induce an immune response in the subject. The immune response may comprise production of neutralizing antibodies against at least one HIV virus or pseudovirus and/or an HIV specific T cell response.

The invention further provides a method of preventing a disease associated with HIV-1 infection in a subject, comprising administering to the subject an effective amount of at least one polypeptide, nucleic acid, vector, virus, virus-like particle, pseudovirus, cell or population of cells of the invention, or any combination thereof of any of the foregoing, sufficient to prevent or inhibit the disease.

In another aspect, the invention provides a method of inducing an immune response against HIV-1 in a subject, comprising administering to the subject an amount of a nucleic acid of the invention effective to induce the immune response, wherein the nucleic acid is operably linked to a promoter sequence that controls the expression of said nucleic acid, and the polynucleotide is present in an effective amount such that uptake of the polynucleotide into one or more cells of the subject and sufficient expression of the nucleic acid occurs to produce to induce the immune response.

In another aspect, the invention provides an isolated antibody or antisera which specifically binds a polypeptide comprising a polypeptide sequence selected from the group consisting of SEQ ID NOS:1-21 and 56-63. Also provided is an antibody or antiserum produced by administering an effective amount of a polypeptide of the invention to a subject (e.g., mammal). The invention further provides an immortalized cell line that produces an antibody of the invention.

In another aspect, the invention provides a method of producing a polypeptide, the method comprising: (a) introducing into a population of cells a nucleic acid of the invention, wherein the nucleic acid is operatively linked to a regulatory sequence effective to produce the polypeptide encoded by the nucleic acid; (b) culturing the cells in a culture medium to produce the polypeptide; and (c) isolating the polypeptide from the cells or culture medium.

Also provided is a method of producing a polypeptide, comprising (a) introducing into a population of cells a recombinant expression vector comprising the nucleic acid of the invention; (b) culturing the cells in a culture medium under conditions sufficient to produce the polypeptide encoded by the nucleic acid; and (c) isolating the polypeptide from the cells or culture medium. In another aspect, the invention includes a method of producing a polypeptide, comprising: (a) introducing into a population of cells a recombinant expression vector comprising the nucleic acid of the invention; (b) administering the vector into a mammal; and (c) isolating the polypeptide from the mammal or from a byproduct of the mammal. The invention also includes an immortalized cell line that produces at least one polypeptide of the invention.

In another aspect, the invention provides a method of generating a cytotoxic T cell response in a subject, comprising administering to the subject an amount of a vector effective to generate said response, wherein the vector comprises a nucleotide sequence encoding at least one polypeptide of the invention, wherein the nucleotide sequence is under the control of a promoter that is capable of expressing the polypeptide in the host. Also included is a method of generating a cytotoxic T cell response in a subject, the method comprising administering to the subject an amount of at least one polypeptide of the invention effective to induce the cytotoxic T cell response.

In a further aspect, the invention provides for the use of at least one polypeptide or nucleic acid of the invention for the manufacture of a medicament for inducing an immune response against at least one HIV virus (e.g., HIV-1) of the same or different subtypes. The medicament may inhibit or prevent infection of cells by at least one HIV-1 virus by inducing an immune response against the at least one HIV-1 virus. The immune response may comprise a neutralizing antibody response or a T cell response against the HIV-1 virus(es).

The invention also provides for the use of at least one polypeptide, nucleic acid, vector, virus-like particle, or pseudovirus of the invention for the preparation of a medicament for inhibiting or preventing infection of cells by at least one HIV virus, including one or more HIV-1 viruses of the same or different subtypes, to which an effective amount of the polypeptide or nucleic acid is contacted or administered. In some aspects, the medicament inhibits or prevents infection of cells of the subject by at least one HIV-1 virus by inducing a neutralizing antibody response and/or a T cell response against one or more HIV-1 viruses of the same or different subtypes.

Additional aspects of the invention are described below.

### BRIEF DESCRIPTION OF THE FIGURES

FIG. 1 presents a schematic representation of an exemplary DNA plasmid expression vector for expression of a recombinant HIV-1 gp120 wild-type (WT) polypeptide or recombinant gp120 polypeptide variant of the invention. This vector, which is termed a “pMAmp” vector, comprises the following components: (1) a CMV enhancer (E); (2) a CMV intermediate-early promoter and (optionally) a CMV intron; (3) a human tissue plasminogen activator (“tPA”) signal sequence (MDAMKRGLCCVLLLCGAVFVSPS) (SEQ ID NO:52); (4) a peptide sequence, ASSGS(H)₆GGSTG (SEQ ID NO:99), which encodes a histidine tag sequence comprising an amino acid sequence of 6 histidine residues (His tag) and two Glycine-Serine linkers (the linker at the N-terminus of the (His)₆ tag sequence comprises amino acid residues SGS, and the linker at the C-terminus of the (His)₆ tag sequence comprises amino acid residues GGS); (5) NheI and AgeI restriction sites that flank the peptide sequence ASSGS(H)₆GGSTG (SEQ ID NO:99) (the amino acids created by introduction of the restrictions sites are underlined in FIG. 1); (6) an open reading frame (ORF) flanked by AgeI and NgoMIV restriction sites; (7) a bovine growth hormone (bGH) polyadenylation sequence; (8) an ampicillin resistance gene sequence (Amp^{R}); and (9) a prokaryotic origin of replication sequence from plasmid pUC (pUC ori). The NheI and AgeI restriction sites comprise amino acid residues AS and TG, respectively. The NgoMIV restriction site comprises amino acid residues AG. The open reading frame comprises a nucleic acid sequence encoding a polypeptide of interest. such as a recombinant WT gp120 polypeptide or a recombinant gp120 polypeptide variant of the invention (such as, for example, a WT gp120 full-length polypeptide or a gp120 full-length polypeptide variant, a WT gp120 Core polypeptide or a gp120 Core polypeptide variant, a WT gp120 Core+V3 polypeptide or a gp120 Core+V3 polypeptide variant, a WT gp120ΔV3 polypeptide or a gp120ΔV3 polypeptide variant, a WT gp120ΔV1V2V3 polypeptide or a gp120ΔV1V2V3 polypeptide variant, a WT gp120 Core+V1V2 polypeptide or a gp120 Core+V1V2 polypeptide variant). This plasmid vector can be used for expression of any gp120 polypeptide sequence of the invention.

FIG. 2 shows exemplary antigenic profiles of fifteen representative gp120 full-length polypeptide variants and four WT parental HIV-1 gp120 polypeptides analyzed using single-point dilution dot-immunoblotting method. Culture supernatants were obtained from transient transfection of CHO-K1 cells with a plasmid vector pMAmp comprising a DNA sequence encoding a gp120 full-length polypeptide variant or parental gp120 full-length polypeptide derived from JRCSF, 93US073, 92US727, and 89.6. The transfection supernatants were immobilized on four replicate dot blots and analyzed for percent binding activity to each of four human monoclonal antibodies (mAbs)—2G12, b3, b6 and b12. In this example, the codons of the DNA sequences encoding the gp120 polypeptide variants and WT parental HIV-1 gp120 polypeptides were not optimized for expression in human cells. The plasmid vector pMAmp without a gp120 gene (designated “Empty Vector”) served as a negative control. The percent binding activity of each indicated polypeptide to each of the four mAbs was calculated by normalizing the binding signal of transfection supernatants to that of the JRCSF gp120 transfection control on the same blot. The b3, b6, and b12 mAbs were obtained from The Scripps Research Institute (San Diego, Calif.), and 2G12 was obtained from POLYMUN Scientific, Vienna, Austria.

FIG. 3 shows exemplary antigenic profiles of seven representative recombinant gp120 core polypeptide variants and four recombinant WT parental HIV-1 gp120 core polypeptides analyzed using single-point dilution dot-immunoblotting method. Culture supernatants were obtained from transient transfection of CHO-K1 cells with a plasmid vector pMAmp comprising a DNA sequence encoding a recombinant gp120 core polypeptide variant or gp120 core polypeptide derived from JRCSF, 93US073, 92US727, and 89.6. The transfection supernatants were immobilized on replicate dot blots and analyzed for percent binding activity to three human mAbs—b3, b6 and b12. In this example, the codons of the DNA sequences encoding the gp120 polypeptide variants and WT parental HIV-1 gp120 polypeptides were not optimized for expression in human cells. The plasmid vector pMAmp without a gp120 gene (designated “Empty Vector”) served as a negative control. The percent binding activity of each indicated core polypeptide to each of the three mAbs was calculated by normalizing the binding signal of transfection supernatants to that of the JRCSF gp120 transfection control on the same blot.

FIG. 4 illustrates representative serial-dilution dot-immunoblot analyses of novel gp120 full-length polypeptide variants. Plasmid vectors encoding each of three gp120 variants, ST-080, ST-140, and ST-194, and the parental HIV-1 JRCSF gp120 polypeptide were transiently transfected into CHO-K1 cells in triplicate. After a 1:2 serial dilution of the supernatant from each transfection, four replicate blots were prepared and reacted with mAbs b3, b6, b12, and 2G12. The binding signals in arbitrary units were quantitated and the averages were plotted as a function of supernatant volume. The error bar indicates the standard deviation of binding signal at each dilution.

FIG. 5 presents a comparison of relative binding signals from immunoprecipitation with those from a dot-blot characterization. Twelve gp120 full-length polypeptide variants were analyzed using ³⁵S-Met/Cys metabolic labeling followed by immunoprecipitation using mAbs b3, b6, and b12. The total amount of gp120 expressed from each construct was measured by precipitating the labeled proteins with a saturating level of a mouse polyclonal antiserum to gp120. Arbitrary binding signals to human mAbs were first normalized to expression levels represented by the binding signal to the polyclonal anti-gp120. For each human mAb, the expression-normalized binding signal for each variant was normalized to that of a JRCSF gp120 polypeptide control to obtain the binding activity relative to the JRCSF gp120 polypeptide, which was set as 1. The binding activity relative to the JRCSF gp120 polypeptide for each gp120 polypeptide variant by dot blot was obtained from the experiment described in FIG. 2.

FIG. 6 illustrates an immunoprecipitation analysis of the interaction of representative gp120 core polypeptide variants with human mAbs b3, b6, and b12. The amount of radiolabeled gp120 core polypeptide variants precipitated by the three representative monoclonal antibodies shown in this figure was determined by immunoprecipitation and then normalized to the total amount of labeled gp120 core polypeptide variants determined by dot-immunoblotting method using a monoclonal anti-His tag antibody. For each human mAb, the expression-normalized binding signal for each variant was normalized to that of a JRCSF gp120 core polypeptide control to obtain the binding activity relative to JRCSF gp120 core polypeptide, which was set as 1.

FIG. 7 presents exemplary surface plasmon resonance sensor data for kinetic analysis of antigen-antibody interactions. The sensor data pertain to the interaction of a representative gp120 full-length polypeptide variant or a parental JRCSF gp120 polypeptide with mAb IgG1b12 or IgG b6. Goat anti-human gamma chain antibody (GAH) was immobilized on a CM5 chip and used to capture human mAbs b6 and b12. A range of concentrations of purified gp120 (0 to 200 nM) was injected onto the antibody surface as described in the Examples below. Sensorgrams for each association and dissociation cycle of the different gp120 concentrations were recorded on the Biacore 2000 instrument (GE Healthcare). The raw sensor data shown here were prepared for kinetic analysis by subtracting the binding response collected from a GAH reference surface. Time (X axis) is represented in units of seconds(s). The association and dissociation data were fitted simultaneously to a single-site binding model by using BlAevaluation software from Biacore (GE Healthcare).

FIG. 8 presents exemplary surface plasmon resonance sensor data for kinetic analysis of representative gp120 core polypeptides to human mAbs IgG1b12 and IgG b6. Sensor data pertain to the interaction of a recombinant gp120 core polypeptide variant or JRCSF gp120 core polypeptide with b12 and IgG b6. Goat anti-human gamma chain antibody (GAH) was immobilized on a CM5 chip and used to capture human mAbs b6 and b12. Sensorgrams for each association and dissociation cycle for a range of 10 different gp120 concentrations ranging from 0 to 200 nM that were injected onto the antibody surface in random order were recorded on the Biacore 2000. The raw sensor data were prepared for kinetic analysis by subtracting the binding response collected from a GAH reference surface. Time (X axis) is represented in units of seconds (s). The association and dissociation data were fitted simultaneously to a single-site binding model by using BIAevaluation software from Biacore (GE Healthcare).

FIG. 9 shows a comparison of association constants (K_{A}) for the interactions between either IgG1b12 or IgG b6 and WT parental JRCSF gp120 full-length polypeptide or a gp120 full-length polypeptide variant (i.e., ST-080, ST-140). Also shown is a comparison of association constants (K_{A}) representing the interactions between either IgG1 b12 or IgG b6 and WT JRCSF gp120 core polypeptide or a gp120 core polypeptide variant (i.e., L7-043, L7-043CDC, L7-098, and L7-098CDC). The association and dissociation data were generated by fitting the sensor data (see, e.g., FIGS. 7 and 8) simultaneously to a single-site binding model using BIAevaluation software from Biacore. K_{A} (M⁻¹) was determined by calculating kₐ/k_{d}, where kₐ is the association rate constant and k_{d} is the disassociation rate constant. The units of kₐ and k_{d} are M⁻¹ s⁻¹ and s⁻¹, respectively. The values indicated by the asterisk (*) were obtained from the best curve fit, however the dissociation rate was extremely slow and exceeded the limit of detection of the Biacore 2000 instrument. N.D.B=No detectable binding.

FIGS. 10A-10F present an alignment of representative recombinant polypeptides of the invention, including gp120 full-length polypeptide variants and gp120 core polypeptide variants, with the known polypeptide sequence of HIV-1 gp120-HXB2 (“HXB2 gp120”, SEQ ID NO:54). Amino acid residues of each polypeptide of the invention are numbered by reference to amino acid residues of the gp120-HXB2 polypeptide. The sequences of the exemplary gp120 full-length polypeptide variants and gp120 core polypeptide variants of the invention shown in this figure are identified herein as follows: ST-003, SEQ ID NO:56; ST-008, SEQ ID NO:1; ST-057, SEQ ID NO:57; ST-168, SEQ ID NO:59; ST-199, SEQ ID NO:62; ST-173, SEQ ID NO:60; ST-128, SEQ ID NO:58; ST-161, SEQ ID NO:6; ST-051, SEQ ID NO:24; ST-080, SEQ ID NO:25; ST-140, SEQ ID NO:4; ST-194, SEQ ID NO:61; ST-148, SEQ ID NO:5; ST-188, SEQ ID NO:7; ST-272, SEQ ID NO:63; L7-068, SEQ ID NO:11; L7-084, SEQ ID NO:12; L7-098, SEQ ID NO:13; L7-010, SEQ ID NO:8; L7-043, SEQ ID NO:10; L7-028, SEQ ID NO:9; and L7-105, SEQ ID NO:14.

FIGS. 11A-11C show characterization of gp120 degradation by CHO-K1 cells. CHO-K1 cells stably expressing the JRCSF gp120 were expanded into a roller bottle and protein production was carried out in serum-free CHO III (A) medium for 14 days. FIG. 11A illustrates the monitoring of gp120 degradation through a 14-day period of roller bottle production. Days are shown along the top of the Western blot in FIG. 11A. Culture supernatant was harvested and replaced with fresh serum-free medium every 24 hours during the 14-day period. Ten microliters (μl) of the supernatant aliquots were analyzed by Western blot using a mouse polyclonal anti-gp120 serum generated against HIV-1 gp120 IIIB (Protein Sciences). The solid bar to the left of each blot indicates intact gp120 polypeptide and the open bars indicate degradation products. FIG. 11B presents a comparison of expression media on gp120 degradation. Roller-bottle production of gp120 using two different serum-free media (CHO III (A) and Opti-MEM I) was performed as described in Example 3 below. Supernatants before or after a 30-fold concentration (designated 1 and 30 in FIG. 11B, respectively) were incubated at room temperature for 6 hours and then at 4° C. overnight. Equal amounts, corresponding to 10 μl of the starting volume of supernatant, were analyzed by Western blot using the polyclonal anti-gp120 serum. FIG. 11C illustrates a protease inhibitor test. An aliquot of the day 13 supernatant (FIG. 11A) was pre-incubated with individual or pooled protease inhibitors at different concentrations as indicated. They were then concentrated 100-fold, and incubated at room temperature for 16 hours. The terms “Low” and “High” indicate that inhibitors were pooled at their lower and higher concentrations, respectively, as in individual tests. Equal amounts, corresponding to 10 μl of the original supernatant volume, were analyzed by Western blot using the polyclonal anti-gp120 serum. Lane numbers are shown at the bottom of the blot. Non-inhibitor controls with and without the 100-fold concentration were loaded in lanes 2 and 1, respectively. For all analyses shown in FIGS. 11A-11C, Invitrogen SeeBlue pre-stained protein standards were used as molecular weight markers; molecular weights in kiloDalton (kDa)) are shown on the right side of all three plates.

FIG. 12 shows the percent neutralization of an SF162 HIV-1 pseudovirus by rabbit sera obtained from rabbits immunized with a DNA plasmid vector (pMAmp vector) into which individual chimeric gp120 genes expressing gp120 full-length polypeptide variants had been cloned. Two rabbits were immunized with each clone using the immunization procedures described in Example 5. An empty pMAmp vector (labeled as “pMAmp” in FIG. 12) served as a control. The larger value of the neutralizing activity for the two rabbits for each clone is presented in the histogram.

FIG. 13 presents the results of pseudovirus-based neutralization assays using rabbit sera induced by the JRCSF gp120 full-length polypeptide and by five representative shuffled (chimeric) gp120 full-length variants: ST-008, ST-051, ST-148, ST-161 and ST-188. A stacked-column representation is shown of the neutralization activity (expressed as percent inhibition of infectivity) of nine pseudoviruses (as indicated in the right-hand legend) by a given rabbit sera (as indicated on the x-axis) used at a 1:7.5 dilution.

FIG. 14 presents a comparison of the neutralization activity induced by the ST-008 chimeric gp120 polypeptide variant and the JRCSF gp120 polypeptide. Eight rabbits were immunized with each construct as described in Example 5. Briefly, a plasmid encoding a selected gp120 variant was used to immunize 8 rabbits and the neutralization activity against a particular HIV-1 pseudovirus was measured. Each rabbit received three DNA injections at days 0, 28 and 56 followed at day 84 by one protein boost adjuvanted with alum with purified JRCSF gp120. Rabbit sera were collected at days 70 and 98 and evaluated for neutralizing activity against a panel of HIV-1 pseudoviruses (Table 10). Neutralizing titers (IC50) are plotted on a log scale. The vertical gray lines separate the results for each virus. The viruses are presented along the x-axis from left to right in order of increasing resistance to neutralization (Table 10). The subtype of each non-clade B HIV-1 virus is indicated in parentheses following its strain name. The aMLV controls for each serum were all negative (IC50≦10; not shown). Solid gray triangles designate individual titers of the rabbits immunized with ST-008 gp120, and the solid black circles represent individual titers of the rabbits immunized with JRCSF gp120. The geometric mean titer (GMT) for each set of sera is shown as a black horizontal bar. For statistical analysis, a two-tailed, two-sample equal-variance Student's t-Test was performed for each virus by using log₁₀ transformations of the IC50 values. Different levels of statistical significance are shown as follows: ** p<0.01; * 0.01<p<0.05; ↑0.05<p<0.10. The gray asterisks and arrows indicate that the titer of the ST-008 sera is higher than that of JRCSF sera for a given virus.

FIG. 15 is a table presenting the results of the neutralization assay of eleven pseudoviruses by antibodies induced by a parental JRCSF gp120 core polypeptide and by selected gp120 core polypeptide variants.

FIG. 16 presents a phylogenetic tree constructed using the amino acid sequences of 15 chimeric full-length gp120 polypeptide variants and ten full-length wild-type parental gp120 polypeptides. The amino acid sequences of these ten parental gp120 polypeptides are set forth in SEQ ID NOS:80-89. The amino acid sequences of the parental gp120 polypeptides and chimeric gp120 polypeptide variants were analyzed by MEGA 3.1 software to construct phylogenetic relationships using the neighbor-joining algorithm. The ten parental gp120 sequences are indicated by white letters in solid black boxes; the twelve gp120 variants are indicated by black letters. Filled circles next to particular chimeric sequence names indicate the most immunogenic sequences. This tree demonstrates that chimeric gp120 variants of the invention exhibiting similar properties are genetically diverse.

FIGS. 17A-B demonstrate that the autologous neutralization response against HIV-1 strain JRCSF is independent of the V3 domain as well as the V1/V2 domains. FIG. 17A provides a schematic representation of three envelope constructs of JRCSF: JRCSF full-length gp120, JRCSF gp120 Core, and JRCSF gp120 Core+V3. FIG. 17B shows the neutralizing antibody responses against various HIV-1 pseudoviruses elicited by the three JRCSF gp120-based immunogen constructs. Each immunogen construct was used to immunize eight rabbits. Day 98 sera were analyzed against a panel of eight pseudoviruses. For each pseudovirus tested (indicated on the y-axis of each plot), the IC50 neutralization titers of antisera raised against the JRCSF full-length gp120 immunogen (“JRCSF gp120”, solid squares), the JRCSF gp120 Core immunogen (“JRCSF Core”, open triangles), and the JRCSF gp120 Core+V3 immunogen (“JRCSF Core+V3”, closed triangles) were plotted, with horizontal bars representing the geometric mean of the IC50 titer (GMT) for each immunogen. A two tailed homostedastic t-test was used to examine differences between each immunogen using log 10 values of IC50 titers. *, 0.01<p<0.05; **, 0.001<p<0.01; and ***, p<0.001.

FIGS. 18A-B demonstrate the role of the V4 and V5 regions in the autologous neutralization response against HIV-1 JRCSF. FIG. 18A shows a schematic representation of the various wild-type and mutant Env sequences which were used to generate pseudoviruses for the cell-based neutralization assay. JRFL, wild-type JRFL Env from Monogram Biosciences; MB JRCSF, wild-type JRCSF Env from Monogram Biosciences; MV JRCSF, wild-type JRCSF Env prepared by the inventors; MV tPA-JRCSF, wild-type JRCSF Env with tPA leader sequences prepared by the inventors; JRCSF-V4(FL), wild-type JRCSF Env with the V4 domain replaced with the V4 domain of JRFL; JRCSF-V5(FL), wild-type JRCSF Env with the V5 domain replaced with the V5 domain of JRFL. FIG. 18B demonstrates that an autologous neutralization epitope against HIV-1_{JRCSF} is located in the V5 domain. Two rabbits were each immunized with the JRCSF gp120 Core immunogen construct as described above, and IgG was purified from the resulting antisera. The two IgG preparations, Anti-Core IgG #1 and Anti-Core IgG #2, were tested for neutralization activity against the pseudoviruses shown in FIG. 18A. Broadly neutralizing anti-HIV human plasma and the human neutralizing mAb b12 were used as positive controls, and the human non-neutralizing mAb b6 was used as a negative control.

FIGS. 19A-B demonstrate that substitution in V4 with heterologous sequences affects the sensitivity to autologous neutralization against JRCSF. The neutralization activities of rabbit serum preparations raised against various JRCSF-based immunogens (JRCSF gp120, JRCSF gp140, or JRCSF Core, as indicated on the x-axis) were characterized against pseudoviruses prepared from the five Env constructs described in FIG. 18A (nd=not done).

FIGS. 20A-C show that rabbit serum with strong autologous neutralizing activity against JRCSF has antibodies which bind specifically to the V5 domain of JRCSF. FIG. 20A shows a schematic representation of two Hepatitis B surface antigen (HbsAg)-V5 constructs, VLP-4000 and VLP-4300, in which the V5 domain sequence of JRCSF was inserted at different positions within the immunodominant “a” loop of HBsAg M. VLP-0800 is an HBsAg M control. FIG. 20B shows that anti-JRCSF Core rabbit serum contains antibodies which bind strongly to the denatured HBsAg-V5 polypeptides. Plasmids pMV-0800, pMV-4000, pMV-4300 were transiently transfected into COS-7 cells. Supernatant, with or without digestion with PNGase F, was separated on reducing SDS-PAGE and blotted onto a nitrocellulose membrane, which was then probed with a control anti-HBsAg antibody and with an anti-JRCSF rabbit serum with strong autologous neutralization titer. FIG. 20C demonstrates that rabbit serum binds strongly to the native HBsAg-V5 in VLP form. VLPs in the supernatant from B were precipitated by ultracentrafugation, suspended, and coated onto a 96-well plate. ELISA was performed using the anti-HBsAg antibodies and the anti-JRCSF rabbit serum as in B. Data are averages of two independent transfections.

FIGS. 21A-B demonstrate that Gly and Ser substitutions (“GS substitutions”) in V5 abolished the autologous neutralization response against HIV-1_{JRCSF}. FIG. 21A shows a schematic representation of three GS substitution mutant constructs of JRCSF gp120 core polypeptides. JRCSF Core (V4-GS2) has two GS substitutions in V4, JRCSF Core (V4-GS8) has eight GS substitutions in V4, and JRCSF Core (V5-GS3) has three GS substitutions in V5, as indicated. FIG. 21B shows the neutralization responses elicited by the GS substitution mutants. Each construct was used to immunize 8 or 10 rabbits via 3×DNA+protein boost as described in Example 5. Day 98 Sera were analyzed against a panel of three pseudoviruses. IC50 titers against each pseudovirus were plotted in a dot plot, with the name of the pseudovirus indicated on the y-axis. Horizontal bars represent the geometric means of IC50 titers of eight rabbits immunized with the same construct. Two tailed homostedastic t-test used to examine difference between any GS substitution construct and JRCSF Core using Log 10 values of IC50 titers. *, 0.01<p<0.05; **, 0.001<p<0.01; and ***, p<0.001.

FIGS. 22A-B demonstrate that ST-008 deletion constructs induce stronger neutralization responses than their JRCSF counterparts. FIG. 22A shows a schematic representation of four deletion constructs based on JRCSF gp120 and ST-008 gp120. Since the N-terminal portion of the ST-008 sequence (containing the C1, V1/V2, and C2 domains) is derived through in vitro recombination from clade B sequences other than JRCSF, that region of the ST-008 sequence is depicted with a different filling pattern than the C-terminal portion of that sequence (containing the V3, C3, V4, C4, V5 and C5 domains) which is identical to that portion of the JRCSF sequence. FIG. 22B shows a comparison of the neutralization responses against three heterologous HIV-1 pseudoviruses SF162, NL43 and BAL (shown on the x-axis) by the four sets of deletion constructs. IC50 titers are plotted in a dot plot, with the unfilled circles representing neutralization by the ST-008 immunogen constructs and the semi-filled diamonds representing neutralization by the JRCSF immunogen constructs. Horizontal bars represent the geometric means of IC50 titers of eight rabbits immunized with the same construct. Two tailed homostedastic t-test was used to examine differences between the matching JRCSF and ST-008 pairs using Log 10 values of IC50 titers. *, 0.01<p<0.05; **, 0.001<p<0.01; and ***, p<0.001.

FIGS. 23A-B demonstrate that ST-008 gp120ΔV1V2V3 is a highly immunogenic construct for induction of an autologous neutralization response against the JRCSF virus. FIG. 23A shows the same schematic representation of the JRCSF gp120 and ST-008 gp120 deletion constructs as were shown in FIG. 22A, and is repeated here for convenience. FIG. 23B shows the autologous neutralization responses against the JRCSF virus elicited by the full-length gp120 and deletion constructs shown in A. The IC50 titer against the Day 98 serum from each rabbit was plotted in a dot plot. The neutralization data of the corresponding immunogen constructs from JRCSF and ST-008 were plotted side by side for comparison, with the unfilled circles representing neutralization by the ST-008 immunogens and the semi-filled diamonds representing neutralization by the JRCSF immunogens. Horizontal bars represent the geometric means of IC50 titers of eight rabbits immunized with the same construct.

FIGS. 24A-C demonstrate the generation of deglycosylation variants through DNA shuffling. FIG. 24A shows the known effects of ten N-glycosylation sites on the HIV-1 Env. FIG. 24B provides a schematic representation of glycosylation patterns at the ten sites for both parental and ten exemplary shuffled JRCSF gp140 variants. JRCSFall, parent gene encoding a fully glycosylated JRCSFgp140; JRCSFnull, a mutant parent gene encoding JRCSF gp140 with N→Q substitutions at all 10 N-glycosylation sites. FIG. 24C shows a comparison of the theoretical glycosylation combinatorics and the experimental outcome from DNA shuffling of JRCSFall and JRCSFnull.

FIGS. 25A-B demonstrate that the deglycosylation JRCSF gp140 variants induced significantly improved autologous neutralizing response against HIV-1_{JRCSF}. FIG. 25A shows a histogram ranking of the neutralization activities on JRCSF. Day 98 sera from 306 rabbits immunized with 100 deglycosylation variants of JRCSF gp140 were analyzed for their IC50 neutralization titer against the JRCSF pseudovirus. The arrow indicates the highest neutralization IC50 value from antisera obtained from a rabbit immunized with the parental JRCSF all immunogen construct. FIG. 25B provides an analysis of the three best gp140 glycosylation variants which induced statistically higher neutralization activity against the JRCSF pseudovirus than did the parent JRCSFall immunogen construct. Sera from these constructs were analyzed against a panel of five pseudoviruses as well as MLV as a negative control. The data are the average of six rabbits for JRCSFall and the averages of three rabbits for the deglycosylation variants and for JRCSFnull.

FIGS. 26A-C demonstrate that a deglycosylation mutation in V5 region enhances the immunogenicity of the autologous neutralizing epitope against HIV-1_{JRCSF} in various forms of JRCSF Env. FIG. 26A provides a schematic representation of three forms of Env, gp140 (which contains gp120 plus gp41); gp120, and Core. FIG. 26B shows a schematic representation of the glycosylation patterns at the ten N-glycan sites for the JRCSF gp140, gp120, and Core variants. Notably, with V1, V2, and V3 deleted, the Core constructs were lacking three out of the 10 potential glycosylation sites. FIG. 26C provides a comparison of the neutralization responses against four pseudoviruses, two pseudoviruses generated from the wildtype JRCSF envelope gene (MB JRCSF and MV JRCSF) and two pseudoviruses with either the JRCSF V4 domain or V5 domain swapped with the corresponding V4 or V5 domain from JRFL (JRCSF-V4(FL) and JRCSF-V5(FL), respectively); see FIG. 18. The average log 10 IC50 titer and standard deviation were calculated from eight rabbits per group. Two tailed homostedastic t-test was performed to test if a glycovariant induces a stronger autologous neutralization response compared to the wt (denoted by an asterisk under the variant name) and if the response was targeting V5 (denoted by an asterisk on top of the JRCSF-V5(FL) column). *, 0.01<p<0.05; **, 0.001<p<0.01; and ***, p<0.001.

FIGS. 27A-B demonstrate that deglycosylation of the V5 epitope renders a JRCSF pseudovirus more sensitive to neutralization. FIG. 27A provides a schematic representation of a pseudovirus containing the wt JRCSF gp120 coding region and a pseudovirus a containing the wt JRCSF gp120 coding region with the N461Q mutation. FIG. 27B shows that the N461Q mutation increases the susceptibility of the pseudovirus to neutralization. Pseudoviruses were generated from the JRCSF N461Q Env gene and four other Env gene constructs—wildtype JRCSF Env (MB JRCSF and MV JRCSF), and JRCSF Env with the JRCSF V4 domain or V5 domain swapped with the corresponding V4 or V5 domain from JRFL (JRCSF-V4(FL) and JRCSF-V5(FL), respectively; see FIG. 18. These pseudoviruses were used for neutralization assays testing various antisera. Antisera were obtained from rabbits immunized with different JRCSF Env-based immunogens, as indicated on the x-axis. These antiserum preparations all exhibited autologous neutralization activity against the JRCSF virus. The mAbs b12 and b6 were included in the comparison to show the JRCSF N461Q pseudovirus has overall neutralization properties similar to other pseudoviruses.

FIGS. 28A-B demonstrate that weak autologous neutralization responses induced by various 92HT594 Env forms primarily target the V3 domain. FIG. 28A provides a schematic representation of three envelope constructs of 92HT594: 92HT594 gp120, 92HT594 gp120 Core and 92HT594 gp120 Core+V3. FIG. 28B shows the neutralization activity elicited by the three constructs. Each construct was used to immunize eight rabbits via 3×DNA+protein boost as described in Example 5. Day 98 Sera were analyzed against a panel of eight pseudoviruses. IC50 titers against each pseudovirus were plotted in a dot plot with the name of the pseudovirus indicated on the y-axis. Horizontal bars represent the geometric means of IC50 titers of eight rabbits immunized with the same construct. Two tailed homostedastic t-test was performed to examine difference in the neutralization activities between Core and gp120 immunogens or between Core+V3 and gp120 immunogens using their Log 10 value of IC50 titers. *, 0.01<p<0.05; **, 0.001<p<0.01; and ***, p<0.001.

FIGS. 29A-B demonstrate that shuffled gp120 Cores induce strong autologous neutralization responses against HIV-1_{92HT594} that are independent of domains V1/V2 and V3. FIG. 29A shows a schematic representation of two shuffled Core variants, L7-043 and L7-105, compared to the 92HT594 gp120 Core sequence. Different filling patterns between amino acids 228-290 of the two shuffled Core variants indicate amino acids sequences derived from clade B parents other than 92HT594. Single amino acid mutations which differ from 92HT594 are also indicated. FIG. 29B shows the neutralization responses elicited by the three Core immunogens. Each construct was used to immunize eight rabbits via 3×DNA+protein boost as described in Example 5. Day 98 sera were assayed for neutralization activity against a panel of three pseudoviruses (SF162, NL43, and 92HT594). IC50 titers against each pseudovirus were plotted in a dot plot with the name of the pseudovirus indicated on the y-axis. Horizontal bars represent the geometric means of IC50 titers of eight rabbits immunized with the same construct. Two tailed homostedastic t-test was performed to examine differences between the neutralization activities of the shuffled Core immunogens and the 92HT594 Core immunogen using their Log 10 value of IC50 titers, and no statistically significant differences were found.

FIGS. 30A-B demonstrate the mapping of the autologous neutralization response against HIV-1_{92HT594} to the V4 region. FIG. 30A shows a schematic representation of three L7-043 V4/V5 swap constructs L7-043_V4V5 JR, L7-043_V4 JR and L7-043_V5 JR, compared to the JRCSF gp120 Core, 92HT594 gp120 Core, and L7-043 immunogen constructs. Each construct was used to immunize eight rabbits as described in Example 5. Day 98 sera were assayed for neutralization activity against a panel of four pseudoviruses, SF162, NL43, JRCSF and 92HT594. IC50 titers against each pseudovirus were plotted in a dot plot. Horizontal bars represent the geometric means of IC50 titers of eight rabbits immunized with the same construct. Two tailed homostedastic t-test was performed using log 10 values of IC50 titers. *, 0.01<p<0.05; **, 0.001<p<0.01; and ***, p<0.001.

FIGS. 31A-B demonstrate that the inclusion of a heterologous V3 domain into the L7-043 variant backbone enhances both the autologous neutralization response against HIV-1_{JRCSF} and the V3-dependent neutralization response. FIG. 31A shows a schematic representation of three Core+V3 constructs from JRCSF, 92HT594, and L7-043 (JRCSF Core+V3; 92HT594 Core+V3; and L7-043 Core+V3 JR). FIG. 31B shows the neutralization responses elicited by the three Core+V3 constructs. Day 98 Sera were assayed for neutralization activity against a panel of six pseudoviruses. IC50 titers against each pseudovirus (as indicated on the y-axis) were plotted in a dot plot. Horizontal bars represent the geometric means of IC50 titers of eight rabbits immunized with the same construct. Two tailed homostedastic t-test was performed using their log 10 value of IC50 titers. *, 0.01<p<0.05; **, 0.001<p<0.01; ***, p<0.001.

### DETAILED DESCRIPTION OF THE INVENTION

The human immunodeficiency virus (HIV), which is a member of the Retroviridae family, precipitates persistent infections in humans that lead to acquire immunodeficiency syndrome (AIDS). Within the Retroviridae family, HIV is classified as a lentivirus. HIV viruses include HIV type 1 (HIV-1) and HIV type 2 (HIV-2) viruses. Isolates of HIV-1, which is the more pathogenic subtype, have been found to exhibit extensive genetic heterogeneity and variability. Distinct genetic subtypes (also known as “clades”) of HIV-1 have been defined and organized into three groups: M (major), O (outlier), and N (non-M or O). See, e.g., FIELDS' VIROLOGY, Vol. 2, P. 1973 (D. M. Knipe et al. eds., 4^{th} ed. 2001, Raven Press, Ltd., New York) (hereinafter “FIELDS VIROLOGY”), and the ENCYCLOPEDIA OF VIROLOGY (R. G. Webster et al. eds., Academic Press, 2^{nd} ed., 1999). The HIV-1 M group includes over 95% of the virus isolates worldwide and comprises at least eight discrete subtypes or clades, which are designated as subtypes (or clades) A, B, C, D, F, G, H, and J (FIELDS VIROLOGY, supra). The HIV-1 O group includes virus isolates obtained from individuals in Cameroon, Gabon, and Equatorial Guinea; the genomes of the O group HIV-1 viruses have less than 50% nucleic acid sequence identity with the genomes of viruses of the M group. Id. The genetically distinct N group of HIV-1 viruses has been identified in viral isolates of individuals from Cameroon. Id.

HIV is a protein-encapsidated positive-sense RNA virus. The HIV viral genome includes the group-specific antigen (gag), polymerase (pol), and envelope (env) structural genes flanked by long terminal repeats. See, e.g., U.S. Pat. No. 6,099,847. The gag gene encodes the gag precursor protein, Pr55, and the pol gene encodes proteins having enzymatic functions (e.g., protease, reverse transcriptase, and endonuclease/integrase). FIELDS VIROLOGY, supra, at 1975; U.S. Pat. No. 6,099,847. The env gene encodes the envelope glycoprotein precursor (gp160). Id. The HIV genome also includes a number of nonstructural regulatory genes that encode accessory proteins that may be involved in the pathogenesis of viral infection.

An HIV virus can be divided into two major components: the viral core and viral envelope. Id. The viral core consists principally of the proteins encoded by the gag and pol genes and the viral RNA. Id. In immature HIV viruses, the core is made up principally of the uncleaved Pr55 protein. As the virus matures, viral protease cleaves the Pr55 protein and products of pol into functional domains that are of significance in viral entry and replication. The Pr55 protein is processed into the matrix, capsid, nucleocapsid, and p6^{Gag} proteins. Id.

The HIV viral envelope comprises a lipid bilayer derived from the cell surface membrane into which the gp160 glycoprotein is concentrated. The gp160 glycoprotein comprises a 160-kDa polyprotein precursor that is an integral membrane protein; gp160 is anchored to the viral membrane by a domain that ultimately becomes the mature transmembrane envelope protein, gp41. FIELDS VIROLOGY, supra, at 2015; Kwong et al., Nature 393:648-659 (1998). The gp160 protein is eventually proteolytically cleaved by cellular proteases to form the mature transmembrane glycoprotein gp41 and an exterior mature envelope glycoprotein termed gp120. FIELDS VIROLOGY, supra, at 2015. The gp41 and gp120 proteins have molecular weights of approximately 41 and 120 kDa, respectively. The letters “gp” in the original protein name represented “glycoprotein,” because when the HIV-1 gp41 or gp120 proteins are expressed in human cells (or, e.g., other mammalian or yeast cells), they are glycosylated. However, as used herein, neither the term gp120 nor gp41 is limited to a glycoprotein form of that protein. For example, the reference to a wild-type gp120 protein refers to wild-type HIV envelope protein having a molecular weight of about 120 kDa, which may or may not be glycosylated depending upon the cell in which it is expressed. The gp120 envelope protein is typically shed from the surface of the envelope complex. Id.

The HIV envelope glycoproteins comprise epitopes that induce immune responses that are important in the design of HIV vaccines, prophylactic and therapeutic treatment methods for inducing immune responses against HIV viruses, and diagnostic methods. The HIV envelope glycoprotein is the main target of neutralizing antibodies. That neutralizing epitopes exist on the envelope (env) has been demonstrated by the isolation from HIV-infected individuals of monoclonal antibodies that can broadly cross-neutralize the virus. Ho et al., Cell 110:135-138 (2002).

The HIV-1 viral envelope glycoproteins project from the outer lipid membrane of the virus. The envelope glycoproteins are organized into oligomeric “spikes” that are displayed on the surface of the virus and are believed to facilitate entry of the virus into host cells. Kwong et al., Nature 393:648-659 (1998). The oligomeric structure is believed to be a trimeric structure, and the surfaces of the oligomeric spikes are composed principally of the gp120 envelope protein. Id. The structure of the wild-type gp120 protein has been well characterized. Id.; Wyatt et al., Nature 393:705-711 (1998). The gp120 protein comprises five variable regions, termed V1-V5 respectively, and five constant or conserved regions, termed C1C5, respectively. For example, C1 represents the first constant region, and V1 represents the first variable region. The gp120 constant and variable regions of the HIV-1 virus are typically highly glycosylated. The constant regions are interspersed among the variable regions. In a linear representation, the HIV-1 gp120 Env glycoprotein comprises the following regions from the N terminal to the C terminal: C1-V1-V2-C2-V3-C3-V4-C4-V5-C5. FIELDS VIROLOGY, supra, at 2016. The cysteine residues are present in the gp120s of different isolates are highly conserved and form disulfide bonds. Id. Through these disulfide bonds, the first four variable regions form large loops that are exposed on the virion surface; the loops are anchored at their bases by the disulfide bonds. Kwong et al., Nature 393 at 648; Leonard et al., J. Biol. Chem. 265:10373-10382 (1990).

To facilitate the identification of the position number or precise location of an amino acid residue in any HIV protein, including, e.g., an HIV wild-type or recombinant protein, or any polypeptide of the invention, the guidelines outlined by Bette Korber et al., “Numbering Positions in HIV Relative to HXB2,” as shown in the HIV Sequence Database of the publicly available government Los Alamos HIV Sequence Database, at the website designated by the URL “hiv.lanl.gov/content/hiv-db/mainpage.html”, are typically used. See also the HIV Sequence Compendium (2002) available on that website. Such guidelines can also used to facilitate the identification of the position number or precise location of a nucleic acid residue in any HIV nucleic acid sequence. Under these guidelines, HXB2 is the reference HIV strain. HXB2 was selected as the prototype virus because it is the most commonly used HIV reference strain for a variety of functional studies. Korber et al., Id. Significantly, the envelope structural data published to date translates residue numbers into the HXB2 numbering scheme. Korber et al., Id. The full-length genome of HIV HXB2 is set forth at GenBank Accession No. K03455. The polypeptide sequence of the gp120 envelope protein of HXB2 (gp120-HXB2) is provided herein as SEQ ID NO:54.

The C1-V1-V2-C2-V3-C3-V4-C4-V5-C5 regions of WT gp120-HXB2 envelope protein (SEQ ID NO:54) can be defined as follows, with amino acid residue positions numbered from the N terminal of the gp120-HBX2 envelope protein (see, e.g., Los Alamos HIV Sequence Database at “hiv.lanl.gov/content/hiv-db/mainpage.html”; see also Willey et al., Proc. Natl. Acad. Sci. 83:5038-42 (1986); Modrow et al., J. Virol. 61:570-8 (1987)):

| Signal peptide | 1-29 or 1-28* |
|---|---|
| C1 | 30-130 or 29-130 |
| V1 | 131-157 |
| V2 | 157-196 |
| C2 | 197-295 |
| V3 | 296-331 |
| C3 | 332-384 |
| V4 | 385-418 |
| C4 | 419-459 or 419-460** |
| V5 | 460-471 or 461-471 |
| C5 | 472-511 |

*In one aspect, the signal peptide comprises amino acid residues 1-28, in which case C1 begins at residue 29; in another aspect, the signal peptide comprises residues 1-29 and C1 begins at residue 30.
**In one aspect, V5 begins at amino acid residue 461 (see Los Alamos HIV Sequence Database noted above), in which case C4 ends at residue 460; in another aspect, V5 begins at residue 460, in which case C4 ends at residue 259. Residue 157 is shared by V1/V2.

Entry of an HIV virus into a target cell is brought about by binding of the gp120 glycoprotein to a CD4 glycoprotein, the major cell-surface receptor for HIV. FIELDS VIROLOGY, supra, at 2018. CD4, which is a 55-kDa member of the immunoglobulin (Ig) superfamily, serves to stabilize the interaction between the T-cell receptor on the surface of T lymphocytes and MHC II molecules present on the surface of antigen-presenting cells. Id. Binding of the gp120 glycoprotein to the CD4 receptor promotes attachment of the virus to the target cell and induces conformational changes in the gp120 protein, facilitating the exposure or formation of a binding site for specific chemokine receptors (e.g., CCR5 and CXCR4). Kwong et al., Nature 393 at 648.

The gp120 envelope glycoprotein is important for preventing or inhibiting HIV infection, as it induces antibodies that neutralize the virus. However, this glycoprotein also elicits antibodies that do not neutralize the virus. Antibodies with viral neutralizing activity antibodies that are induced during HIV infection are believed to recognize conserved or variable epitopes of the gp120 glycoprotein positioned at or near the receptor-binding regions. Id. Induced non-neutralizing antibodies are directed against regions of the gp120 protein that obstructed in the trimeric configuration and that only become exposed when the protein is shed from the envelope complex. Wyatt et al., Nature 393 at 705. The variability and possibly glycosylation of the surface of the gp120 glycoprotein likely modulate the immunogenicity and antigenicity of the gp120 glycoprotein. Kwong et al., Nature 393 at 648.

An HIV-1 gp120 core envelope protein has been defined based on X-ray crystallography. Kwong et al., Nature 393:648-659 (1998); Wyatt et al., Nature 393:705-711 (1998). In brief, the gp120 core protein (glycosylated or unglycosylated) is a gp120 derivative that lacks the V1, V2 and V3 variable loops and selected amino- and carboxy-terminal amino acid residues. Reference to a gp120 core polypeptide thus implies a protein construct that does not have the V1, V2 and V3 loops and lacks some amino- and carboxy-terminal amino acid residues. In contrast, the complete or “full-length” gp120 protein includes the V1/V2 and V3 loops and amino- and carboxy-terminal amino acid residues. The gp120 core protein binds the CD4 receptor. Id.

HIV induces a variety of host immune responses in a subject infected with the virus, including HIV-specific humoral and cellular immune responses. The humoral responses include the production of antibodies that neutralize HIV infectivity. Neutralizing antibodies assist in inhibition or control of in vivo viral replication and/or may facilitate a lowering of the level of plasma viremia associated with primary infection. FIELDS VIROLOGY, supra, at 2051. Neutralizing antibodies typically bind to the virus and prevent its attachment to target cells. Neutralizing antibodies may be specific for a particular viral isolate or specific for a group or broad range of viral isolates. That neutralizing antibodies are important in regulating or impeding the course of HIV infection is suggested by the large number of HIV viral variants that resist neutralization which have emerged in an effort by the virus to evade the infected subject's immune response. Id.

Cellular immune responses against HIV include MHC class I HIV-specific CD8+ cytotoxic T lymphocyte (CTL) responses against particular HIV proteins, such as the envelope proteins (e.g., gp120). The HIV-specific CTL response is believed to be involved in the down-regulation of viremia after infection and to slow the progression of disease. Id. at 2052. A CTL response assists in controlling viral replication. HIV proteins also elicit CD4+ T cell responses. For example, HIV proteins include helper T-cell epitopes that can be presented by MHC class II alleles. Id. at 2053. The recognition of CD4+ T cells by such epitopes may induce the production of cytokines. CD4+T-helper cell responses are also associated with inhibition of viral replication and/or a decrease of plasma viremia. Id.

Timely development of an effective prophylactic vaccine to HIV-1 is important for countering the current AIDS epidemic. The results from passive transfer of broadly cross-reactive HIV neutralizing antibodies, which can protect monkeys against SHIV challenge, have demonstrated the potential power of a prophylactic vaccine based on the induction of neutralizing antibodies. Mascola, J. R. et al., Nat Med 6:207-10 (2000); Parren, P. W. et al., J. Virol. 75:8340-7 (2001). As the only viral protein targeted by neutralizing antibodies isolated from AIDS patients, the HIV-1 envelope glycoprotein complex can be an important component of vaccine candidates. Moore, J. P. et al., J. Virol. 75:5721-9 (2001); Srivastava, I. K. et al., J. Virol. 77:11244-59 (2003); Yang, X., et al., J. Virol. 76:4634-42 (2002). The existence of conserved neutralizing epitopes on the viral envelope protein is demonstrated by the existence of broadly neutralizing monoclonal antibodies isolated from infected individuals that recognize conformational epitopes defined by monoclonals b12 and 2G12 (Calarese, D. A. et al., Science 300:2065-71 (2003); Saphire, E. O. et al., Science 293:1155-9 (2001)), or continuous epitopes targeted by Z13, 2F5, 4E10, and 447-52D (Sharon, M., et al., Structure (Camb) 11:225-36 (2003); Zwick, M. B. et al., J. Virol. 75:10892-905 (2001)).

However, to date, the elicitation of broadly crossing neutralizing antibodies by experimental vaccines is extremely rare. Notably, this invention includes recombinant gp120 nucleic acid variants and gp120 polypeptide variants encoded therefrom that are capable of inducing immune responses to human immunodeficiency viruses, in particular an HIV-1 viruses. These immune responses include the production of cross-reactive neutralizing antibodies against such HIV viruses, in particular HIV-1 viruses of the same or different subtypes or clades.

### DEFINITIONS

It is also to be understood that the terminology used herein is for the purpose of describing particular embodiments only and is not intended to be limiting. Unless defined otherwise, all technical and scientific terms used herein have the same meaning as commonly understood by one of ordinary skill in the art to which the invention pertains.

The terms “nucleic acid” and “polynucleotide” are used interchangeably to refer to a polymer of nucleic acid residues (e.g., deoxyribonucleotides or ribonucleotides) in either single- or double-stranded form. Unless specifically limited, the terms encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605 2608 (1985); and Cassol et al. (1992); Rossolini et al., Mol. Cell. Probes 8:91 98 (1994)). The term nucleic acid or polynucleotide is used interchangeably with cDNA or mRNA encoded by a gene.

The term “gene” broadly refers to any nucleic acid segment (e.g., DNA) associated with a biological function. A gene may include a coding sequence and/or regulatory sequence required for their expression. A gene may also include non-expressed DNA nucleic acid segment(s) that, e.g., form recognition sequences for other protein(s) (e.g., promoter, enhancer, or other regulatory region). A gene can be obtained from a variety of sources, including cloning from a source of interest or synthesizing from known or predicted sequence information, and may include one or more sequences designed to have desired parameters.

The terms “polypeptide,” “peptide,” and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

Numbering of a given amino acid polymer or nucleic acid polymer “corresponds to” or is “relative to” the numbering of a selected amino acid polymer or nucleic acid polymer when the position of any given polymer component (e.g., amino acid, nucleotide, also referred to generically as a “residue”) is designated by reference to the same or an equivalent position in the selected amino acid or nucleic acid polymer, rather than by the actual numerical position of the component in the given polymer. Thus, for example, the numbering of a given amino acid position in a given polypeptide sequence corresponds to the same or equivalent amino acid position in a selected polypeptide sequence used as a reference sequence.

An “equivalent position” (for example, an “equivalent amino acid position” or “equivalent nucleic acid position” or “equivalent residue position”) is defined herein as a position (such as an amino acid position or nucleic acid position or residue position) of a test polypeptide (or test polynucleotide) sequence which aligns with a corresponding position of a reference polypeptide (or reference polynucleotide) sequence, when aligned (preferably optimally aligned) using an alignment algorithm as described herein. The equivalent amino acid position of the test polypeptide sequence need not have the same numerical position number as the corresponding position of the test polypeptide. Likewise, the equivalent nucleic acid position of the test polynucleotide sequence need not have the same numerical position number as the corresponding position of the test polynucleotide.

A polypeptide “variant” comprises a polypeptide sequence that differs in one or more amino acid residues from the polypeptide sequence of a parent or reference polypeptide. For example, a polypeptide variant may comprise a sequence which differs from a parent or reference polypeptide sequence in up to 30% of the total number of residues of the parent or reference polypeptide sequence, such as in up to 25% or 20% of the residues, e.g., in up to 15%, 12%, 10%, 9%, 8%, 7%, 6% 4%, 3%, 2%, or 1% of the total number of residues of the parent or reference polypeptide sequence. A polypeptide “variant” of a parent or reference polypeptide sequence may comprise a polypeptide sequence that differs from the parent or reference sequence in at least 1 to 100 or more amino acid residues (e.g., 1, 2, 3, 4, 5, 6, 7, 8, 9, 10, 11, 12, 13, 14, 15, 16, 17, 18, 19, 20, 25, etc. or more amino acid residues). A polypeptide variant may differ from a parent or reference polypeptide by, e.g., deletion, addition, or substitution of one or more amino acid residues of the parent or reference polypeptide, or any combination of such deletion(s), addition(s), and/or substitution(s). A polypeptide variant usually exhibits at least about 70% or 80%, and preferably at least about 90%, 91%, 92%, 93%, 94%, 95%, 96%, 97%, 98%, 99% or more amino acid sequence identity to a parent or reference polypeptide sequence.

A nucleic acid “variant” comprises a nucleic acid sequence comprising a nucleotide sequence that differs in one or more nucleic acid residues the nucleotide sequence of a parent or reference nucleic acid. For example, a nucleic acid variant may comprise a sequence which differs from a parent or reference nucleic acid sequence in up to 30% of the total number of residues of the parent or reference sequence, such as in up to 25% or 20% of the residues, e.g., in up to 15%, 12%, 10%, 9%, 8%, 7%, 6% 4%, 3%, 2%, or 1% of the total number of residues of the parent or reference nucleic acid sequence. A nucleic acid “variant” of a parent or reference nucleic acid sequence may comprise a nucleotide sequence that differs from the parent or reference sequence in at least 1 to 100 or more nucleotide residues (e.g., 1, 2, 3, 4, 5, 6, 7, 8, 9, 10, 11, 12, 13, 14, 15, 20, 25, etc. or more nucleic acid residues). A nucleic acid variant may differ from a patent or reference nucleic acid, by e.g., deletion, addition, or substitution of one or more nucleic acid residues parent or reference nucleic acid, or any combination of such deletion(s), addition(s), and/or substitution(s). A nucleic acid variant usually exhibits at least about 70% or 80%, and preferably at least about 90%, 91%, 92%, 93%, 94%, 95%, 96%, 97%, 98%, 99% or more nucleotide sequence identity to a parent or reference nucleic acid sequence.

“Naturally occurring” as applied to an object refers to the fact that the object can be found in nature as distinct from being artificially produced by man. “Non-naturally occurring” as applied to an object means the object is not naturally occurring (i.e., that the object cannot be found in nature). For example, a non-naturally occurring polypeptide refers to a polypeptide that has been prepared by man, such as, for example, by being synthesized in vitro or prepared artificially.

A “subsequence” or “fragment” of a sequence of interest is any portion of the entire sequence, up to but not including the entire sequence of interest.

A nucleic acid, protein or other component is “isolated” when it is partially or completely separated from components with which it is normally associated (other proteins, nucleic acids, cells, synthetic reagents, etc.). On a molar basis, an isolated species is more abundant than other species in a composition. For example, an isolated species may comprise at least about 50%, 70%, 80%, or 90% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a protein or nucleic acid sample, followed by visualization upon staining. If desired, a high-resolution technique, such as high performance liquid chromatography (HPLC) or a similar means can be utilized for purification of the material.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 75%, 80%, 85%, 90%, 95%, 97%, or 99% pure (e.g., percent by weight on a molar basis).

In a related sense, the invention provides methods of enriching compositions for such molecules. A composition is enriched for a molecule when there is a substantial increase in the concentration of the molecule after application of a purification or enrichment technique. A substantially pure polypeptide or polynucleotide will typically comprise at least about 55%, 60%, 70%, 80%, 85%, 90%, 95%, 97%, 98, or 99% percent by weight (on a molar basis) of all macromolecular species in a particular composition.

A nucleic acid or polypeptide is “recombinant” when it is artificial or engineered, or derived from an artificial or engineered protein or nucleic acid.

The term “recombinant” when used with reference to a cell typically indicates that the cell replicates a heterologous nucleic acid or expresses a polypeptide encoded by a heterologous nucleic acid. Recombinant cells can comprise genes that are not found within the native (non-recombinant) form of the cell. Recombinant cells also include those that comprise genes that are found in the native form of the cell, but are modified and re-introduced into the cell by artificial means. The term also encompasses cells that comprise a nucleic acid endogenous to the cell that has been modified without removing the nucleic acid from the cell; such modifications include those obtained by gene replacement, site-specific mutation, and related techniques known to those of ordinary skill in the art. Recombinant DNA technology includes techniques for the production of recombinant DNA in vitro and transfer of the recombinant DNA into cells where it may be expressed or propagated, thereby producing a recombinant polypeptide.

A “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include at least promoters and optionally transcription termination signals. Typically, the recombinant expression cassette includes a nucleic acid to be transcribed (e.g., a nucleic acid encoding a desired polypeptide) and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

An “exogenous” nucleic acid,” “exogenous DNA segment,” “heterologous sequence,” or “heterologous nucleic acid,” as used herein, is one that originates from a source foreign to the particular host cell, or, if from the same source, is modified from its original form. Thus, a heterologous gene in a host cell includes a gene that is endogenous to the particular host cell, but has been modified. Modification of a heterologous sequence in the applications described herein typically occurs through the use of directed molecular evolution methods. Thus, the terms refer to a DNA segment which is foreign or heterologous to the cell, or homologous to the cell but in a position within the host cell nucleic acid in which the element is not ordinarily found. Exogenous nucleic acids or exogenous DNA are expressed to yield exogenous polypeptides.

A “vector” may be any agent that is able to deliver or maintain a nucleic acid in a host cell and includes, for example, plasmids, naked nucleic acids, viral vectors, viruses, nucleic acids complexed with one or more polypeptide or other molecules, as well as nucleic acids immobilized onto solid phase particles. Vectors are described in detail below. A vector can be useful as an agent for delivering or maintaining an exogenous gene and/or protein in a host cell. A vector may be capable of transducing, transfecting, or transforming a cell, thereby causing the cell to replicate or express nucleic acids and/or proteins other than those native to the cell or in a manner not native to the cell. A vector may include materials to aid in achieving entry of a nucleic acid into the cell, such as a viral particle, liposome, protein coating, or the like. Any method of transferring a nucleic acid into the cell may be used; unless otherwise indicated, the term vector does not imply any particular method of delivering a nucleic acid into a cell or imply that any particular cell type is the subject of transduction. The present invention is not limited to any specific vector for delivery of a gp120 polypeptide variant-encoding nucleic acid and/or gp120 polypeptide variant.

The term “expression vector” typically refers to a nucleic acid construct or sequence, generated recombinantly or synthetically, with a series of specific nucleic acid elements that permit transcription of a particular nucleic acid in a host cell. The expression vector typically includes a nucleic acid to be transcribed operably linked to a promoter. The term “expression” includes any step involved in the production of the polypeptide including, but not limited to, transcription, post-transcriptional modification, translation, post-translational modification, and/or secretion.

A “signal peptide” is a peptide sequence that typically proceeds a polypeptide of interest and is translated in conjunction with the polypeptide and directs or facilitates the polypeptide to the secretory system. A signal peptide is typically cleaved from the polypeptide of interest following translation.

The term “encoding” refers to the ability of a nucleotide sequence to code for one or more amino acids. The term does not require a start or stop codon. An amino acid sequence can be encoded in any one of six different reading frames provided by a polynucleotide sequence and its complement.

The term “control sequence” is defined herein to include all components, which are necessary or advantageous for the expression of a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, a control sequence includes a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “coding sequence” is refers to a nucleotide sequence that directly specifies the amino acid sequence of its protein product. The boundaries of the coding sequence are generally determined by an open reading frame (ORF), which may begin with the ATG start codon.

A nucleic acid is “operably linked” with another nucleic acid sequence when it is placed into a functional relationship with another nucleic acid sequence. For instance, a promoter or enhancer is operably linked to a coding sequence if it directs transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

A “host cell” is any cell that is susceptible to transformation with a nucleic acid.

“Substantially the entire length of a polynucleotide sequence” or “substantially the entire length of a polypeptide sequence” refers to at least about 50%, generally at least about 60%, 70%, or 75%, usually at least about 80%, 85%, 90%, 91%, 92%, 93%, 94%, 95%, 96%, 97%, 98%, 99%, 99.5% or more of the length of a polynucleotide sequence or polypeptide sequence, respectively.

An “antigen” refers to a substance that reacts with the product(s) of an immune response stimulated by a specific immunogen. See, e.g., Julius Cruse et al., ATLAS OF IMMUNOLOGY 60 (1999); Richard Coico et al., IMMUNOLOGY: A SHORT COURSE 27-30 (5^{th} ed. 2003). An immune response may comprise a humoral response and/or a cell-mediated immune response (e.g., cytotoxic T lymphocytes (CTLs)). Product(s) of an immune response may include antibodies and/or CTLs. Antigens are typically macromolecules (e.g., polypeptides, nucleic acids, complex carbohydrates, phospholipids, polysaccharides) that are foreign to the host; that portion of the antigen known as the antigenic determinant reacts with (e.g., binds to) the product(s) of the immune response, such as an antibody or a specific T cell receptor on a T lymphocyte. An antigen may, but not necessarily, induce an immune response as well as react with the product(s) of the immune response. “Antigenicity” refers the state or property of being antigenic—i.e., having the properties of an antigen. Specificity of an antigen may be shown in the relation of an antigen to its antibody or vice versa; an antigen typically reacts in a highly specific fashion with its corresponding antibody and not with the same degree of specificity with other antibodies evoked by the immunogen. An “antigenic amount” is an amount of an antigen that detectably reacts with the product(s) of an immune response stimulated by a specific immunogen.

An “immunogen” is a substance that is capable of inducing an immune response rather than immunological tolerance. See, e.g., Julius Cruse et al., supra at 60-61; Richard Coico, supra at 27-30. Immunogens also reacts with (e.g., bind) the product(s) of the induced immune response that has or have been specifically induced against them. Thus, all immunogens are antigens. “Immunogenicity” refers the state or property of being immunogenic—i.e., having the properties of an immunogen. An “immunogenic amount” is an amount of an immunogen that is effective to induce a detectable an immune response. An immunogen may elicit a strong immune response in a subject, such as at least partial or complete protective immunity to at least one pathogen (e.g., HIV virus).

An “immunomodulator” or “immunomodulatory” molecule, such as an immunomodulatory polypeptide or nucleic acid, modulates an immune response. By “modulation” or “modulating” an immune response is intended that the immune response is altered. For example, “modulation” of or “modulating” an immune response in a subject generally means that an immune response is stimulated, induced, inhibited, decreased, increased, enhanced, or otherwise altered in the subject. Such modulation of an immune response can be assessed by means known to those skilled in the art, including those described below. An “immunostimulator” is a molecule, such as a polypeptide or nucleic acid, that stimulates an immune response.

An “adjuvant” refers to a substance that enhances an immune response. For example, an adjuvant may enhance another substance's immune-stimulating properties (such as, e.g., the immune-stimulating properties of an immunogen) or the pharmacological effect(s) of a compound or drug. An adjuvant may non-specifically enhance the immune response to an antigen. An adjuvant may comprise an oil, emulsifier, killed bacterium, aluminum hydroxide, or calcium phosphate (e.g., in gel form), or any combination of one or more thereof. Examples of adjuvants useful in methods of the invention include “Freund's Complete Adjuvant,” “Freund's incomplete adjuvant,” Alum, CpG, MLP, QS-21, AS02, and other adjuvants described elsewhere herein. Freund's Complete Adjuvant is an emulsion of oil and water containing an immunogen, an emulsifying agent and mycobacteria. Freund's Incomplete Adjuvant is the same, but without mycobacteria. An adjuvant is typically administered to a subject in an amount sufficient to show a detectable enhancement of an immune response.

As used herein, an “antibody” (abbreviated “Ab”) refers to an immunoglobulin (abbreviated “Ig”), whether natural or wholly or partially synthetically produced. The term includes all derivatives thereof that maintain specific binding ability. The term also covers any protein having a binding domain that is homologous or largely homologous to an immunoglobulin binding domain. Such proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. Derivatives of the IgG class, however, are preferred in the present invention. A typical antibody structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains, respectively.

The term “antibody fragment” refers to any derivative of an antibody that is less than full-length. Typically, the antibody fragment retains at least a significant portion of the full-length antibody's specific binding ability. Examples of antibody fragments include, but are not limited to, e.g., Fab, Fab′, F(ab′)₂, scFv, Fv, dsFv diabody, and Fd fragments. An antibody fragment may be produced by any means known in the art. For instance, the antibody fragment may be enzymatically or chemically produced by fragmentation of an intact antibody or it may be recombinantly produced from a gene encoding the partial antibody sequence. For example, fragments of antibodies can be produced by digestion with a peptidase. For example, pepsin digests an antibody below the disulfide linkages in the hinge region to produce F(ab)′₂, a dimer of a Fab fragment which itself is a light chain joined to VH-CH1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially a Fab fragment with part of the hinge region. The Fc portion of the antibody molecule corresponds largely to the constant region of the immunoglobulin heavy chain, and is responsible for the antibody's effector function (see FUNDAMENTAL IMMUNOLOGY, W. E. Paul, ed., Raven Press, N.Y. (1993) for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term also includes antibody fragments either produced by the modification of whole antibodies or synthesized de novo using recombinant DNA methodologies.

Alternatively, the antibody fragment may be wholly or partially synthetically produced. The antibody fragment may optionally be a single chain antibody fragment. Alternatively, the fragment may comprise multiple chains that are linked together, for instance, by disulfide linkages. The fragment may also optionally be a multimolecular complex. A functional antibody fragment will typically comprise at least about 50 amino acids and more typically will comprise at least about 200 amino acids.

Monoclonal or polyclonal antibodies can be prepared any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of the invention. In addition, transgenic mice or other organisms, including mammals, may be used to express humanized antibodies. Phage display technology can be used to identify antibodies and heteromeric Fab fragments that specifically bind to selected antigens (see, e.g., McCafferty et al., Nature 348:552 554 (1990); Marks et al., Biotechnology 10:779 783 (1992)).

The term “epitope” refers to an antigenic determinant capable of specific binding to a part of an antibody. Epitopes usually consist of chemically active surface groupings of molecules such as amino acids or sugar side chains and usually have specific three-dimensional structural characteristics, as well as specific charge characteristics. Conformational and nonconformational epitopes are distinguished in that the binding to the former but not the latter is lost in the presence of denaturing solvents.

A “specific binding affinity” between two molecules, e.g., a ligand and a receptor, means a preferential binding of one molecule for another. The binding of molecules is typically considered specific if the binding affinity (e.g., K_{A}) is about 1×10² M⁻¹ to about 1×10¹² M⁻¹ (i.e., about 10⁻² M to 10⁻¹² M) or greater, including about 10⁴ to 10¹¹ M⁻¹, about 10⁶ to 10¹⁰ M⁻¹, about 10⁸ M⁻¹ to 10¹⁰ M⁻¹ or about 10⁸ to 10⁹ M. The binding affinity of a ligand and a receptor (e.g., such as between an antibody and an antigen) may be measured by standard techniques known to those of skill in the art. Values of K_{A} for the binding interaction between an antigen and an antibody typically range from about 10⁵ M⁻¹ to about 10¹¹ M⁻¹, usually about 10⁷ M⁻¹ to about 10⁹ M⁻¹, and often about 10⁸ M. Non-limiting examples of well-known techniques for measuring binding affinities of molecules include, e.g., surface plasmon resonance such as Biacore technology (GE Healthcare) as discussed elsewhere herein, isothermal titration microcalorimetry (MicroCal LLC, Northampton, Mass. USA), ELISA, and FACS. For example, FACS or other sorting methods may be used to select for populations of molecules (such as for example, cell surface-displayed ligands) that specifically bind to the associated binding pair member (such as a receptor, e.g., a soluble receptor). Ligand-receptor complexes may be detected and sorted e.g., by fluorescence (e.g., by reacting the complex with a fluorescent antibody that recognizes the complex). Molecules of interest which bind an associated binding pair member (e.g., receptor) are pooled and re-sorted in the presence of lower concentrations of receptor. By performing multiple rounds sorting in the presence of decreasing concentrations of receptor (an exemplary concentration range being on the order of 10⁻⁶ M down to 10⁻⁹ M, i.e., 1 micromolar (μM) down to 1 nanomolar (nM), or less, depending on the nature of the ligand-receptor interaction), populations of the molecule of interest exhibiting specific binding affinity for the receptor may be isolated.

An “antigen-binding fragment” of an antibody is a peptide or polypeptide fragment of the antibody that binds or selectively binds an antigen. An antigen-binding site is formed by those amino acids of the antibody that contribute to, are involved in, or affect the binding of the antigen. See Scott, T. A. and Mercer, E. I., CONCISE ENCYCLOPEDIA: BIOCHEMISTRY AND MOLECULAR BIOLOGY (de Gruyter, 3d ed. 1997), and Watson, J. D. et al., RECOMBINANT DNA (2d ed. 1992) (hereinafter “Watson”).

The phrase “specifically (or selectively) binds” to an antibody or “specifically (or selectively) immunoreactive with,” when referring to a protein, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Usually, a specific or selective reaction will be at least twice background signal or noise and more typically more than 10 to 100 times background.

The term “cytokine” includes, e.g., but is not limited to, interleukins, interferons, chemokines, hematopoietic growth factors, tumor necrosis factors and transforming growth factors. In general these are small molecular weight proteins that regulate maturation, activation, proliferation, and differentiation of cells of the immune system.

The term “screening” describes, in general, a process that identifies optimal molecules of the present invention, such as, e.g., including polypeptides of the invention, and related fusion proteins comprising the same, and nucleic acids encoding all such molecules. Several properties of the respective molecules can be used in selection and screening, for example, an ability of a respective molecule to induce or alter a desired immune response in a test system or in an in vitro, ex vivo, or in vivo application. “Selection” is a form of screening in which identification and physical separation are achieved simultaneously by expression of a selection marker, which, in some genetic circumstances, allows cells expressing the marker to survive while other cells die (or vice versa). Screening markers include, for example, luciferase, beta-galactosidase and green fluorescent protein, reaction substrates, and the like. Selection markers include drug and toxin resistance genes, and the like. Another mode of selection involves physical sorting based on a detectable event, such as binding of a ligand to a receptor, reaction of a substrate with an enzyme, or any other physical process which can generate a detectable signal either directly (e.g., by utilizing a chromogenic substrate or ligand) or indirectly (e.g., by reacting with a chromogenic secondary antibody). Selection by physical sorting can by accomplished by a variety of methods, such as by FACS in whole cell or microdroplet formats.

In the case of antigens or immunogens, several properties of an antigen or immunogen (such as, e.g., a gp120 polypeptide variant of the invention), can be used in selection and/or screening methods, including antigenicity (e.g., an ability to bind an antibody against HIV), immunogenicity (e.g., ability to induce an immune response against HIV virus or pseudovirus), expression, folding, and/or stability.

Because of limitations in studying primary immune responses in vitro, in vivo studies are particularly useful screening methods. In some such studies, a polynucleotide or polypeptide of the invention is first introduced to test animals, and an induced immune response is subsequently studied by analyzing the type of immune response in the immunized animal (e.g., antibody production in the immunized animal's serum, proliferation of T cells), or by studying the quality or strength of the induced immune response in the immunized animal (e.g., induced antibody titer level).

The term “subject” as used herein includes, but is not limited to, an organism or animal, including mammals and non-mammals. A mammal includes, e.g., but is not limited to, a human, non-human primate (e.g., baboon, orangutan, monkey), mouse, pig, cow, goat, cat, rabbit, rat, guinea pig, hamster, horse, monkey, sheep or other non-human mammal. A non-mammal includes, e.g., but is not limited to, a non-mammalian invertebrate and non-mammalian vertebrate, such as a bird (e.g., a chicken or duck) or a fish.

The term “pharmaceutical composition” refers to a composition suitable for pharmaceutical use in a subject, including an animal or human. A pharmaceutical composition typically comprises an effective amount of an active agent and a carrier or excipient. The carrier is typically a pharmaceutically acceptable carrier or excipient.

The term “effective amount” refers to a dosage (or dose) or amount of a substance sufficient to produce a desired result. The desired result may comprise an objective or subjective improvement in the recipient of the dosage or amount. For example, the desired result may comprise a measurable, detectable or testable induction, promotion, enhancement or modulation of an immune response in a subject to whom a dosage or amount of a particular antigen or immunogen (or composition thereof) has been administered. A dosage (or dose) or amount of an immunogen sufficient to produce such result can be described as an “immunogenic” dosage (or dose) or amount.

A “prophylactic treatment” is a treatment administered to a subject who does not display signs or symptoms of, or displays only early signs or symptoms of, a disease, pathology, or disorder, such that treatment is administered for the purpose of preventing or decreasing the risk of developing the disease, pathology, or disorder. A prophylactic treatment functions as a preventative treatment against a disease, pathology, or disorder, or as a treatment that inhibits or reduces further development or enhancement of a disease, pathology or disorder. For example, a prophylactic treatment may inhibit or limit further infection of a subject by a pathogen or virus or limit or reduce pathogen or viral replication or population (e.g., viral load or titer) in a subject exposed to a pathogen or virus. A “prophylactic activity” is an activity of an agent that, when administered to a subject who does not display signs or symptoms of, or who displays only early signs or symptoms of, a pathology, disease, or disorder, prevents or decreases the risk of the subject developing the pathology, disease, or disorder. A “prophylactically useful” agent (e.g., nucleic acid or polypeptide) refers to an agent that is useful in preventing development of a disease, pathology, or disorder, or useful in inhibiting or reducing further development or enhancement of a disease, pathology or disorder. For example, a “prophylactically useful” agent may be useful in inhibiting or limiting further infection of a subject by a pathogen or virus or limiting or reducing pathogen or viral replication or population (e.g., viral load or titer) in a subject exposed to a pathogen or virus.

A “therapeutic treatment” is a treatment administered to a subject who displays symptoms or signs of pathology, disease, or disorder, in which treatment is administered to the subject for the purpose of diminishing or eliminating those signs or symptoms. A “therapeutic activity” is an activity of an agent that eliminates or diminishes signs or symptoms of pathology, disease or disorder when administered to a subject suffering from such signs or symptoms. A “therapeutically useful” agent means the agent is useful in decreasing, treating, or eliminating signs or symptoms of a disease, pathology, or disorder.

Generally, the nomenclature used herein and many of the laboratory procedures in cell culture, molecular genetics, molecular biology, nucleic acid chemistry, and protein chemistry described below are well known and commonly employed by those of ordinary skill in the art. Standard techniques, such as described in Sambrook et al., Molecular Cloning—A Laboratory Manual (2^{nd} Ed.), Vols. 1-3, Cold Spring Harbor Laboratory, Cold Spring Harbor, N.Y., 1989 (hereinafter “Sambrook”) and CURRENT PROTOCOLS IN MOLECULAR BIOLOGY, F. M. Ausubel et al., eds., Current Protocols, a joint venture between Greene Publishing Associates, Inc. and John Wiley & Sons, Inc. (1994, supplemented through 1999) (hereinafter “Ausubel”), are used for recombinant nucleic acid methods, nucleic acid synthesis, cell culture methods, and transgene incorporation, e.g., electroporation, injection, gene gun, impressing through the skin, and lipofection. Generally, oligonucleotide synthesis and purification steps are performed according to specifications. The techniques and procedures are generally performed according to conventional methods in the art and various general references that are provided throughout this document. The procedures therein are believed to be well known to those of ordinary skill in the art and are provided for the convenience of the reader.

Various additional terms are defined or otherwise characterized herein.

Polypeptides of the Invention

The present invention provides novel recombinant or isolated polypeptides that are capable of inducing an immune response(s) against HIV, particularly against one or more HIV viruses, including against one or more HIV-1 viruses. In one aspect, the invention provides novel isolated or recombinant polypeptides that are capable of inducing a humoral and/or cellular immune response against one or more HIV viruses, including against one or more HIV-1 viruses. In one aspect, the invention provides novel isolated or recombinant polypeptides that are capable of inducing neutralizing antibodies against HIV (HIV neutralizing antibodies), e.g., neutralizing antibodies against one or more HIV-1 viruses. In another aspect, the invention provides novel isolated or recombinant polypeptides that are capable of inducing a specific T cell response against at least one HIV virus, including against at least one HIV-1 virus.

The invention also provides novel isolated or recombinant polypeptides that are capable of binding to or reacting with an antibody against HIV, including binding or reacting with a neutralizing antibody against HIV (HIV neutralizing antibody) and/or a non-neutralizing antibody against HIV (HIV non-neutralizing antibody). In one aspect, the invention includes novel isolated or recombinant polypeptides that bind to or react with an antibody (Ab) against at least one HIV-1 virus. Some such polypeptides specifically bind to or react with a neutralizing Ab against HIV-1 (HIV-1 neutralizing Ab). Some such polypeptides also or alternatively specifically bind or react with a non-neutralizing Ab against HIV-1 (HIV-1 non-neutralizing Ab).

The polypeptides of the invention, including all mentioned above and discussed throughout, are collectively referred to as “polypeptides of the invention.” The polypeptides of the invention include recombinant or isolated gp120 polypeptide variants, such as, e.g., gp120 full-length polypeptide variants, gp120 core polypeptide variants, gp120 core+V1V2 polypeptide variants, gp120 core+V3 polypeptide variants, gp120ΔV3 polypeptide variants and gp120ΔV1V2V3 polypeptide variants, as described in more detail below. The polypeptide variants may be chimeric gp120 polypeptide variants that comprise amino acid residues from a variety of HIV gp120 antigens. Such polypeptides may be termed chimeric gp120 polypeptide variants or chimeric HIV antigens. Some such chimeric gp120 polypeptide variants are capable of inducing an immune response(s) against one or more HIV viruses, particularly against one or more HIV-1 viral strains. Some such polypeptide variants are capable of inducing antibodies against one or more HIV viruses (e.g., one or more HIV-1 viruses); in particular, some such polypeptide variants are capable of inducing neutralizing antibodies against one or more HIV viruses, such as one or more HIV-1 viruses. Some such polypeptide variants are capable of binding to or reacting with an antibody against one or more HIV viruses, including, but not limited to, binding or reacting with a neutralizing antibody against HIV virus, particularly HIV-1. In one aspect, the invention includes novel isolated or recombinant polypeptides that bind to or react with an antibody (Ab) against at least one HIV-1 virus. Some such polypeptides specifically bind to or react with a neutralizing Ab against HIV-1 (HIV-1 neutralizing Ab). Some such polypeptides also or alternatively specifically bind or react with a non-neutralizing Ab against HIV-1 (HIV-1 non-neutralizing Ab).

Also included are proteins (or polypeptides) that comprise at least one polypeptide of the invention and at least one signal peptide. The signal peptide is typically covalently attached or fused to the N terminus of the polypeptide of the invention of interest. The signal sequence facilitates secretion of an expressed protein from a host cell.

Also included are fusion proteins comprising a polypeptide of the invention and a second polypeptide that is covalently attached to the polypeptide of the invention. The second peptide, which typically is not identical to the polypeptide of the invention, may facilitate identification, purification, or solubility of the polypeptide of the invention. The fusion protein may include a linker or “spacer” peptide to facilitate, e.g., proper protein folding.

The invention also includes a conjugate comprising at least one polypeptide of the invention and at least one non-polypeptide moiety covalently attached to the polypeptide. The non-polypeptide moiety may comprise, e.g., a sugar moiety or a polymer molecule. The polymer may be a natural or synthetic homopolymer or heteropolymer, including, e.g., a linear or branched polyethylene glycol (PEG), polyvinyl alcohol (PVA), polycarboxylic acid, or poly-(vinylpyrrolidone). The PEG group may have a molecular weight of from 300 to 100,000 kDa.

Various HIV-1 gp120-envelope-polypeptide-coding sequences or fragments thereof were recursively recombined to form libraries comprising recombinant polynucleotides, from which some polypeptides of the invention were identified. Identified polypeptides include gp120 polypeptide variants of the invention. As discussed in greater detail infra, recombinant gp120 polypeptide variants of the invention include recombinant gp120 full-length polypeptide variants and recombinant gp120 core polypeptide variants. Some such gp120 variants induce a humoral and/or cellular response against one or more HIV-1 strains of the same subtype or different subtypes. Methods for obtaining libraries of recombinant polynucleotides and/or for obtaining diversity in nucleic acids used as the substrates for molecular evolution (e.g., in vitro recombination) are described infra.

As with a wild-type gp120 polypeptide, the position number or precise location of an amino acid residue of a gp120 polypeptide variant of the invention, including a gp120 full-length polypeptide variant or gp120 core polypeptide variant, can be identified by reference to the polypeptide sequence of gp120-HXB2 (identified herein as SEQ ID NO:54). Thus, for example, the position number or precise location of an amino acid residue of a gp120 full-length polypeptide variant or gp120 core polypeptide variant is determined by reference to the gp120-HXB2 polypeptide sequence. Similarly, the position number or precise location of a nucleic acid residue of a nucleic acid of the invention, which encodes a gp120 polypeptide of the invention, can be identified by reference to the nucleic acid sequence that encodes the gp120-HXB2 polypeptide sequence (GenBank Acc. No. K03455).

In one aspect, the present invention provides an isolated or recombinant polypeptide comprising a polypeptide sequence having at least 85%, 86%, 87%, 88%, 89%, 90%, 91%, 92%, 93%, 94%, 95%, 96%, 97%, 98%, 99%, 99.5%, or 100% sequence identity to a polypeptide sequence selected from the group consisting of SEQ ID NOS:1-7 and 56-63, wherein such polypeptide induces an immune response against at least one HIV virus or pseudovirus (e.g., HIV-1) or binds to at least one anti-HIV antibody, such as an anti-HIV-1 antibody. Such polypeptide variants that induce an immune response against at least one HIV-1 virus or pseudovirus or bind to at least one anti-HIV-1 Ab are “full-length” HIV-1 gp120 polypeptide variants, as they have a sequence length approximating that of the full-length WT HIV-1 HXB2-gp120 polypeptide. The induced immune response may comprise a neutralizing antibody response against one or more HIV-1 viruses of the same or of different subtypes.

The invention also provides an isolated or chimeric HIV-1 gp120 polypeptide variant comprising a polypeptide sequence having at least 90%, 91%, 92%, 93%, 94%, 95%, 96%, 97%, 98%, 99%, 99.5%, or 100% sequence identity to any one of SEQ ID NOS:1-7 and 56-63, wherein the polypeptide variant induces the production of antibodies (Abs) against the at least one HIV-1 virus or pseudovirus. The antibodies may be neutralizing antibodies. The neutralizing antibodies may be produced against two or more HIV-1 viruses or pseudoviruses of the same or different subtypes.

In another aspect, the invention provides an isolated or recombinant polypeptide comprising a polypeptide sequence having at least 85%, 86%, 87%, 88%, 89%, 90%, 91%, 92%, 93%, 94%, 95%, 96%, 97%, 98%, 99%, 99.5%, or 100% sequence identity to a polypeptide sequence selected from the group consisting of SEQ ID NOS:15-21, wherein such polypeptide induces an immune response against at least one HIV virus or pseudovirus (e.g., HIV-1) or binds to an anti-HIV Ab, such as an anti-HIV-1 Ab. Such polypeptides that induce an immune response against at least one HIV-1 virus or pseudovirus or bind to an anti-HIV-1 Ab are HIV-1 gp120 core polypeptide variants, as they have a length approximating that of a WT HXB2-gp120 core polypeptide construct.

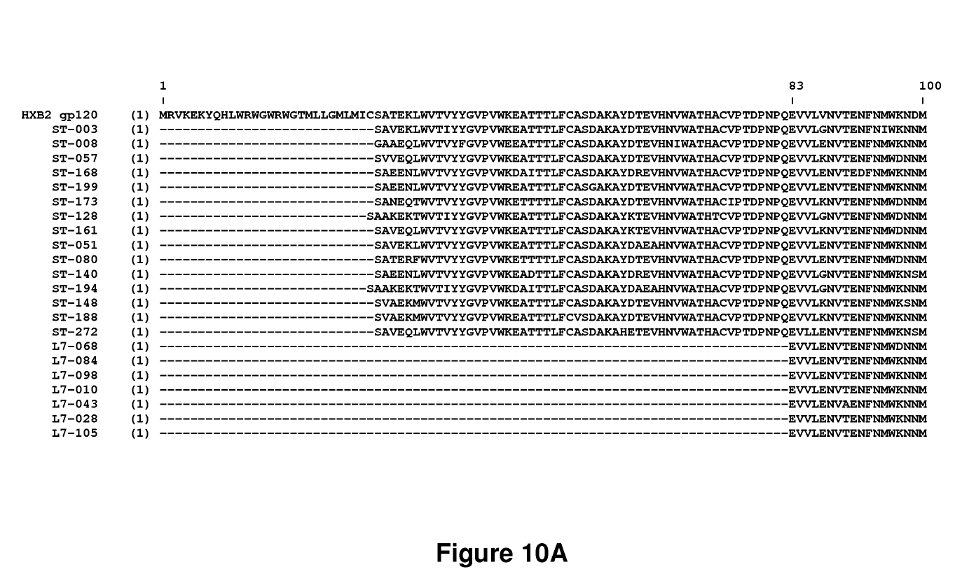

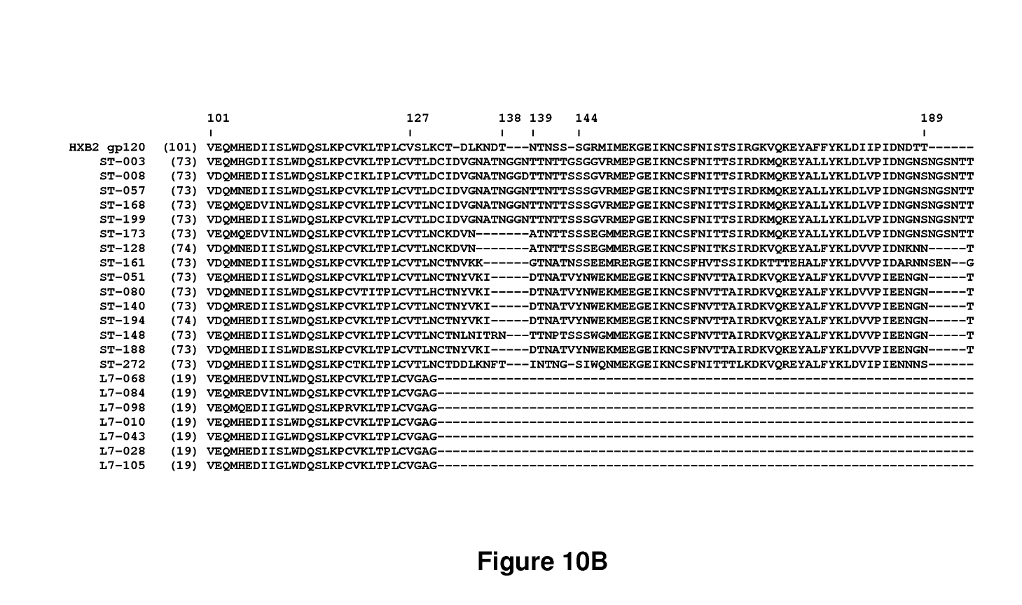

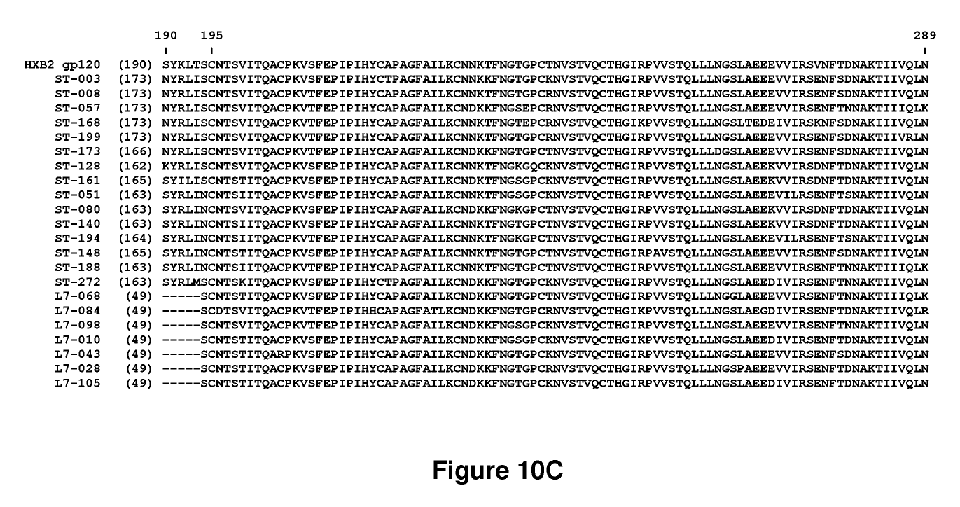

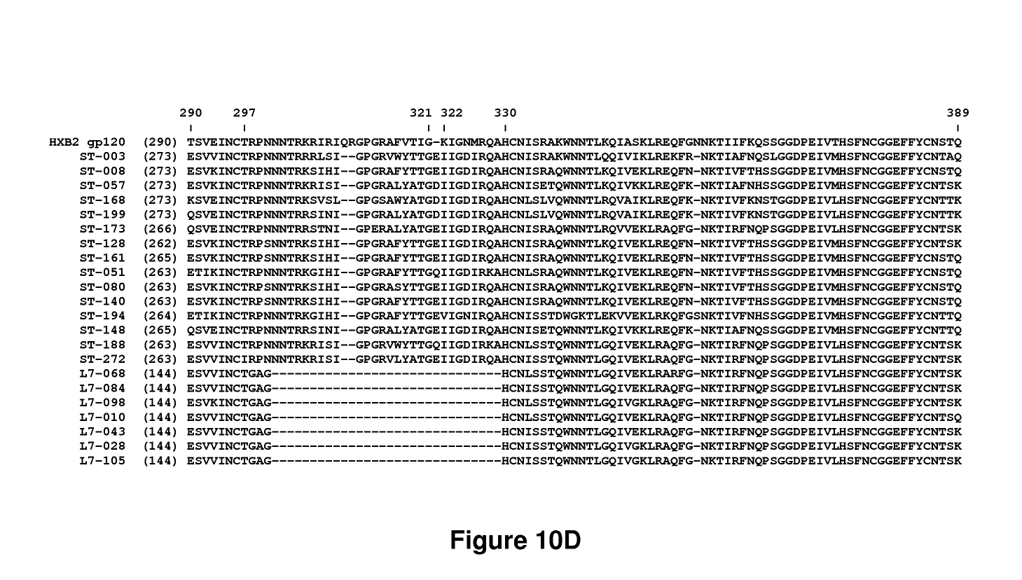

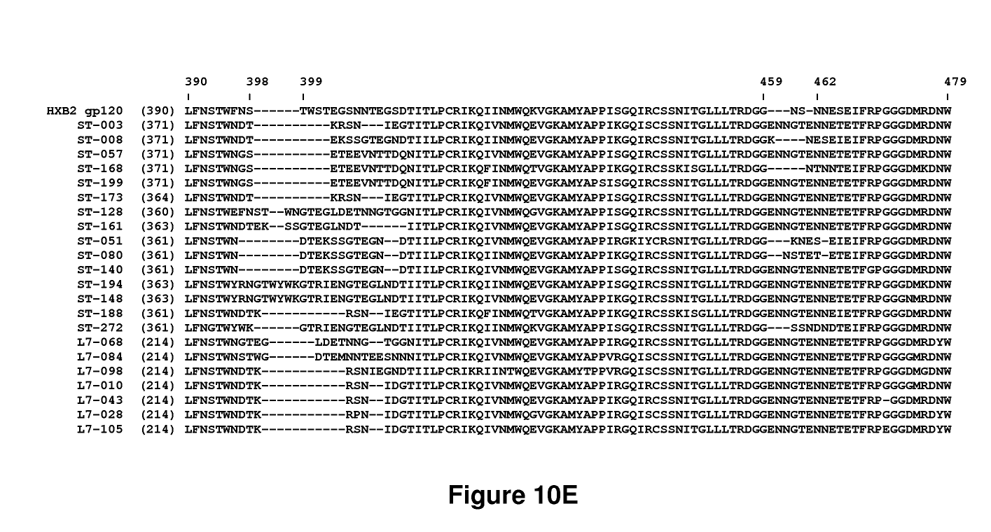

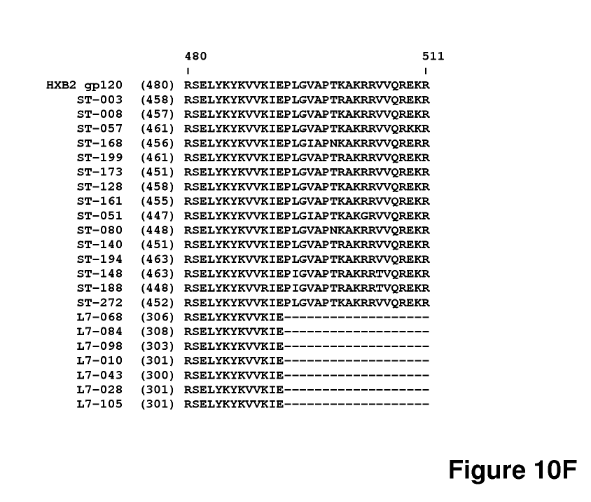

These gp120 core variants include a CDC tail polypeptide at the C terminus, which tail facilitates and/or enhances expression of a gp120 core polypeptide variant. The CDC tail polypeptide comprises a polypeptide sequence comprising 32 amino acid residues. A CDC tail nucleic comprising a polynucleotide sequence encoding a CDC tail polypeptide can be optionally added (e.g., covalently linked) to the C-terminal of a nucleic acid sequence encoding a gp120 core envelope variant. In one embodiment, this CDC tail polypeptide comprises a polypeptide comprising a sequence of 32 amino acids (SEQ ID NO:22), which polypeptide sequence can be optionally added (e.g., covalently linked, such as through a linker, such as a GAG tripeptide linker) to the C-terminus of the polypeptide sequence of a gp120 core variant. For example, a CDC tail comprising the sequence in SEQ ID NO:22 may be added covalently to the C terminus of each exemplary core variant of the invention shown in FIGS. 10A-10F, e.g., to the C terminus of L7-010, L7-028, L7-043, L7-068, L7-084, L7-098, and L7-105 (SEQ ID NOS:11-14, respectively) to produce the sequences identified herein as L7-010CDC, L7-028CDC, L7-043CDC, L7-068CDC, L7-084CDC, L7-098CDC, and L7-105CDC (SEQ ID NOS:15-21, respectively).

In another aspect, the invention provides an isolated or recombinant polypeptide comprising a polypeptide sequence having at least 85%, 86%, 87%, 88%, 89%, 90%, 91%, 92%, 93%, 94%, 95%, 96%, 97%, 98%, 99%, 99.5%, or 100% sequence identity to a polypeptide sequence selected from the group consisting of SEQ ID NOS:8-14, wherein such polypeptide induces an immune response against at least one HIV virus or pseudovirus (e.g., HIV-1) or binds to an anti-HIV Ab, such as an anti-HIV-1 Ab. Such polypeptides are HIV-1 gp120 core polypeptide variants that lack the CDC tail polypeptide at the C terminus.

In another aspect, the invention provides a recombinant or isolated chimeric HIV-1 gp120 polypeptide comprising a polypeptide sequence that differs from the polypeptide sequence of any of the group consisting of SEQ ID NOS:1-21 and 56-63 by no more than 1, 2, 3, 4, 5, 6, 7, 9, 10, 15, 20, or 25 amino acid residues, wherein the polypeptide variant induces the production of neutralizing antibodies against at least one HIV-1 virus in a subject to whom an effective amount of the variant is administered.

The invention provides an isolated or recombinant polypeptide comprising a polypeptide sequence that differs from the polypeptide sequence of any of the group consisting of SEQ ID NOS:1-21 and 56-63 by no more than 1, 2, 3, 4, 5, 6, 7, 9, 10, 15, 20, or 25 amino acid residues, and which includes an amino acid substitution in a glycosylation motif (N-X-S/T) which eliminates N-linked glycosylation at one or more glycosylation sites selected from N156, N188, N197, N276, N295, N301, N332, N386, N448, and N461, wherein the amino acid residues are numbered according to the amino acid residues of the recombinant gp120-HXB2 envelope protein (SEQ ID NO:54) as shown in FIGS. 10A-10F, wherein the polypeptide induces an immune response against at least one HIV virus or pseudovirus.

In one embodiment, the amino acid substitution is a substitution of the N (Asn) in the glycosylation motif with a different amino acid, such as a Q (Gln). In another embodiment, the amino acid substitution is a substitution of the Ser(S) or Thr(T) in the glycosylation motif with a different amino acid, such as an Ala (A). Such deglycosylated polypeptide variant may comprise substitutions which eliminate N-linked glycosylation at 2, 3, 4, 5, 6, 7, 8, 9 or all 10 of said glycosylation sites. In one particular aspect, the deglycosylated polypeptide variant comprises the substitution N461Q. Some such deglycosylated variants induce an increased immune response against at least one human immunodeficiency virus type 1 (HIV-1 virus) or pseudovirus, compared to the immune response induced by the parent polypeptide (i.e., the polypeptide lacking substitutions at the glycosylation sites).

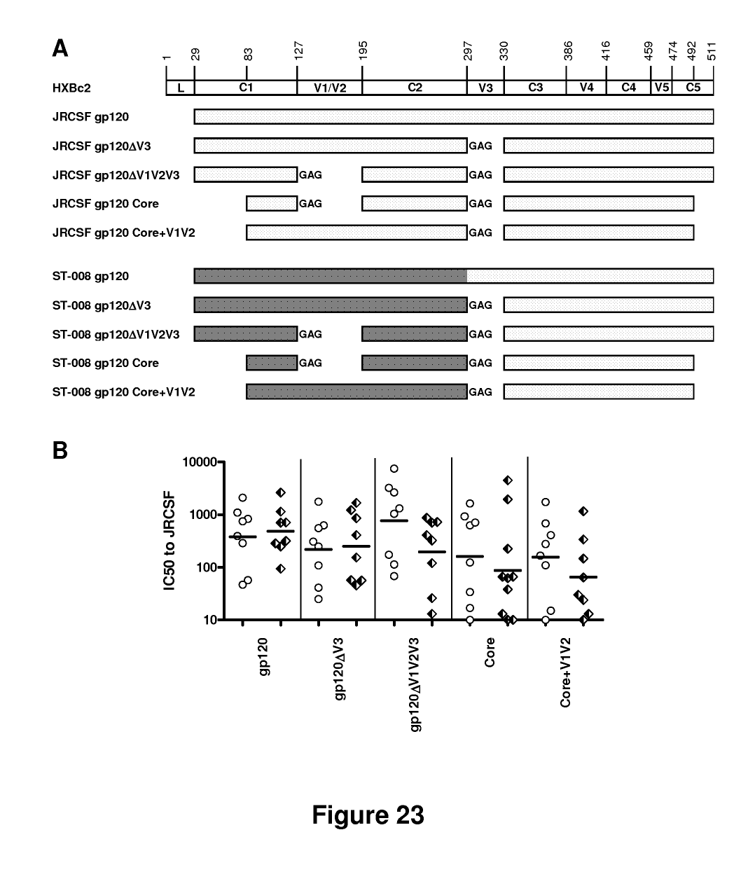

The invention further provides an isolated or recombinant polypeptide comprising a polypeptide fragment of a gp120 full-length polypeptide of the invention (e.g., any of SEQ ID NOS:1-7 and 56-63), wherein the fragment is a core polypeptide construct that binds to an HIV neutralizing antibody and/or induces in a subject to whom an effective amount of it is administered a neutralizing antibody response and/or T cell response against HIV-1. A core polypeptide of any of the gp120 full-length polypeptides of the invention (e.g., SEQ ID NOS:1-7 and 56-63) can be determined by comparison with other core polypeptides of the invention. For example, a core polypeptide of ST-008 (SEQ ID NO:1) comprises those amino acid residues of ST-008 that correspond by alignment to the amino acid residues of a gp120 core polypeptide variant of the invention, such as L7-068 (SEQ ID NO:11), L7-043 (SEQ ID NO:10), etc. (see FIGS. 10A-10F and FIG. 23A). The core polypeptide of ST-008 is identified herein as SEQ ID NO:109.

The invention further provides an isolated or recombinant polypeptide comprising a polypeptide fragment of a gp120 full-length polypeptide of the invention (e.g., any of SEQ ID NOS:1-7 and 56-63), wherein the fragment is a core+V1V2 polypeptide construct that binds to an HIV neutralizing antibody and/or induces in a subject to whom an effective amount of it is administered a neutralizing antibody response and/or T cell response against HIV-1. A core+V1V2 polypeptide of any of the gp120 full-length polypeptides of the invention (e.g., SEQ ID NOS:1-7 and 56-63) can be determined by comparison with other core+V1V2 polypeptides of the invention, such as the core+V1V2 polypeptide fragment of ST-008 (SEQ ID NO:1) which is identified herein as SEQ ID NO:110 (see also FIG. 23A).

The invention further provides an isolated or recombinant polypeptide comprising a polypeptide fragment of a gp120 full-length polypeptide of the invention (e.g., any of SEQ ID NOS:1-7 and 56-63), wherein the fragment is a gp120ΔV1V2V3 polypeptide construct that binds to an HIV neutralizing antibody and/or induces in a subject to whom an effective amount of it is administered a neutralizing antibody response and/or T cell response against HIV-1. A gp120ΔV1V2V3 polypeptide of any of the gp120 full-length polypeptides of the invention (e.g., SEQ ID NOS:1-7 and 56-63) can be determined by comparison with other gp120ΔV1V2V3 polypeptides of the invention, such as the gp120ΔV1V2V3 polypeptide fragment of ST-008 (SEQ ID NO:1) identified herein as SEQ ID NO:108 (see also FIG. 23A).

The invention further provides an isolated or recombinant polypeptide comprising a polypeptide fragment of a gp120 full-length polypeptide of the invention (e.g., any of SEQ ID NOS:1-7 and 56-63), wherein the fragment is a gp120ΔV3 polypeptide construct that binds to an HIV neutralizing antibody and/or induces in a subject to whom an effective amount of it is administered a neutralizing antibody response and/or T cell response against HIV-1. A gp120ΔV3 polypeptide of any of the gp120 full-length polypeptides of the invention (e.g., SEQ ID NOS:1-7 and 56-63) can be determined by comparison with other gp120ΔV3 polypeptides of the invention, such as the gp120ΔV3 polypeptide of ST-008 (SEQ ID NO:1) identified herein as SEQ ID NO:107 (see also FIG. 23A).

Some such gp120 full-length polypeptide variants and gp120 core polypeptide variants and fragments thereof (with or without the CDC tail) discussed above have an ability to induce in a subject to whom an effective amount of such polypeptide(s) is administered an immune response against HIV, e.g., HIV subtype 1 virus (HIV-1). The immune response may comprise a humoral (e.g., antibody) and/or cell-mediated (e.g., T cell) immune response. The immune response may be against one or more HIV viruses, including one or more HIV-1 viruses, and the viruses may be of the same subtype or different subtypes or any combination thereof. Such polypeptides of the invention are useful in prophylactic methods of preventing HIV infection or HIV virus transmission and/or therapeutic methods of treating HIV infection and/or in methods of detecting or diagnosing HIV exposure infection, as described in greater detail elsewhere herein.

Methods for detecting and measuring antibody responses induced by a polypeptide of the invention are well known and described in greater detail below. Methods for detecting and measuring T cell responses, including T cell activation and T cell proliferation, are also known. Briefly, T cell activation is commonly characterized by physiological events including, e.g., T cell-associated cytokine synthesis (e.g., IFN-γ production) and induction of various activation markers such as CD25 (interleukin-2 (IL-2) receptor). CD4+ T cells recognize their immunogenic peptides in the context of MHC class II molecules, whereas CD8+ T cells recognize their immunogenic peptides in the context of MHC class I molecules.

Some such polypeptides of the invention have an ability to induce in the “receiving” subject (a subject to whom an effective amount of at least one such polypeptide or nucleic acid encoding such polypeptide has been administered) an immune response against one or more HIV viruses, which HIV viruses are either of the same HIV virus subtype or different HIV virus subtypes. For example, some such polypeptides are capable of inducing an immune response against two or more HIV viruses of the same subtype (or “clade”), such as HIV-1 subtype B. Some such polypeptides have the ability to induce in the receiving subject an immune response against HIV viruses of multiple different subtypes. For example, some such polypeptides have an ability to induce an immune response in the receiving subject against one or more HIV-1 viruses of two, three, four, five, six, seven or more different HIV-1 subtypes, including, but not limited to, subtypes A, B, C, D, F, G, H, and J.

Some such polypeptides of the invention induce in a subject to whom such polypeptide(s) (or nucleic acid encoding an effective amount of such polypeptide(s)) is administered prior to exposure or transmission of the HIV virus an immune response that is sufficient to inhibit or reduce HIV infection. For example, some such polypeptides are capable of reducing the initial dose of virus transferred (inoculum size) to the subject upon viral transmission, by, e.g., as much as 10%, 20%, 30%, 50%, 60% 70%, 80%, 90%, 95%, 96%, 97%, 98%, or 100%. Such polypeptides are therefore are useful in a prophylactic regimen or method in which an effective amount of the polypeptide(s) (or nucleic acid(s) encoding an effective amount of such polypeptide(s)) is administered to the subject prior to exposure to or transmission of the virus. With such prophylactic treatment regimens, the polypeptide induces in the receiving subject an immune response such that upon subsequent virus transmission to the subject, HIV infection or the HIV virus inoculum size is reduced in the subject (e.g., by the induced humoral and/or T cell response). Some such polypeptides of the invention, when administered in an effective amount, are expected to reduce viral infection or the initial dose of virus transferred (inoculum size) to the subject by at least 10%, 20%, 30%, 50%, 60%, 70%, 80%, 90%, 95%, 97%, 98%, or more. The inoculum size may be reduced such that total viral load and/or the viral set point is reduced, thereby inhibiting or blunting HIV infection—when compared to the viral load and/or set point that would be reached without prior administration of such polypeptide of the invention. With such a reduced viral load or viral set point, the subject would be expected to live longer before developing AIDS or associated diseases. In some instances, the ability of the subject to transmit the virus further to another subject is reduced, since the viral load in the subject has been reduced.

Some such polypeptides of the invention are expected to reduce HIV infection in the subject to whom an effective amount of the polypeptide has been administered and to whom HIV is transmitted by at least 10%, 20%, 30%, 50%, 60%, 70%, 80%, 90%, 95%, 97%, 98%, or more. Such polypeptides are thus are useful in a prophylactic method in which an effective amount of the polypeptide(s) (or nucleic acid(s) encoding an effective amount of such polypeptide(s)) is administered to the subject prior to transmission of the virus. Some such polypeptides of the invention may prevent transmission of HIV, including HIV-1 and multiple HIV-1 viruses of the same or different subtypes or clades. Prevention of transmission of multiple HIV-1 viruses is achieved by the production of cross-reactive neutralizing antibodies against such viruses.

Some such polypeptides of the invention have an ability to induce in a subject who has been immunized with an effective amount of such polypeptide(s) (or nucleic acid encoding such polypeptide(s)) prior to exposure to or transmission of the virus an immune response that provides at least partial or complete immunity to HIV, thereby inhibiting or preventing HIV infection. In one aspect, some such polypeptides of the invention provide partial or complete immunity to or against at least one HIV-1 virus. Such polypeptides are thus useful as a prophylactic vaccine against HIV, including in some instances against one or more HIV-1 viruses. In a further aspect, some such polypeptides of the invention provide partial or complete immunity against multiple (e.g., two or more) HIV-1 viruses of the same subtype. Some polypeptides of the invention provide an HIV neutralizing antibody response and/or HIV-specific T cell response against at least two HIV-1 viruses of the same subtype or at least two HIV-1 viruses of different subtypes. Some such polypeptides of the invention provide partial or complete immunity to multiple HIV-1 viruses of the different subtypes.

Some such polypeptides of the invention are useful in a therapeutic regimen or method for treating a subject to whom an HIV virus has been transmitted. In such regimen or method, the subject is administered an effective amount of at least one such polypeptide of the invention (or nucleic acid(s) encoding an effective amount of such polypeptide) following transmission of the virus. Some such polypeptides induce in subject to whom an HIV virus has been transmitted an immune response that is sufficient to inhibit or reduce further HIV infection. For example, some such polypeptides are capable of reducing the dose of virus transferred (inoculum size) in the subject present following virus transmission, by, e.g., as much as 10%, 20%, 30%, 50%, 60% 70%, 80%, 85%, 90%, 95% or more. A subject to whom the virus has been previously transmitted may be treated by administration of at least one such polypeptide of the invention. Upon administration of an effective amount of at least one such polypeptide to such subject, an immune response (e.g., a humoral and/or T cell response) is sufficiently elicited such that the virus inoculum size, viral load, and/or viral set point is/are reduced. HIV infection is inhibited or blunted. Some such polypeptides of the invention are expected to reduce HIV infection in an HIV-exposed subject, including HIV-1 infection, by at least about 10%, 20%, 30%, 50%, 60%, 70%, 80%, 85%, 90%, 95% or more. With a reduced inoculum size, viral load and/or viral set point, the HIV-exposed subject would be expected to live longer before developing AIDS and/or associated disorders or diseases. In addition, the ability of the HIV-exposed subject to transmit the virus further to another subject would be reduced. Some such polypeptides of the invention may provide a subject to whom an HIV virus has been transmitted least partial or complete immunity against one or more HIV viruses, such as, e.g., HIV-1. Such polypeptides of the invention are believed useful as a therapeutic vaccine against HIV, including in some instances against one or more HIV-1 viruses. Some such polypeptides provide partial or complete immunity against two or more HIV viruses or the same or different subtypes. Some such polypeptides provide an anti-HIV neutralizing Ab response and/or HIV-specific T cell response against at least two HIV viruses of the same subtype or of different subtypes. Some such polypeptides provide a neutralizing antibody response and/or T cell response against at HIV-1 viruses of the same or different subtypes.

Also provided is an isolated or recombinant polypeptide comprising a polypeptide sequence having at least 85%, 86%, 87%, 88%, 89%, 90%, 91%, 92%, 93%, 94%, 95%, 96%, 97%, 98% 99%, or 100% identity to a polypeptide sequence selected from the group consisting of SEQ ID NOS:1-21 and 56-63, wherein the polypeptide induces the production of neutralizing antibodies against at least one HIV virus (e.g., HIV-1 virus) or pseudovirus in a subject to whom an effective amount of the polypeptide is administered. The polypeptide may induce in the subject the production of a titer of HIV-1 neutralizing Abs that is greater than the titer of HIV-1 neutralizing antibodies induced in the subject by recombinant WT HIV-1 gp120 Env polypeptide (e.g., HIV-1_{JRCSF} gp120 polypeptide).

xxx In another aspect, the invention provides an isolated or recombinant chimeric HIV-1 gp120 polypeptide variant comprising a polypeptide sequence having at least 90%, 91%, 92%, 93%, 94%, 95%, 96%, 97%, 98%, 99%, 99.5%, or 100% sequence identity to any one of SEQ ID NOS:1-21 and 56-63, wherein the polypeptide variant induces in the subject to whom an effective amount of the polypeptide is administered production of antibodies capable of binding to at least one HIV virus (e.g., HIV-1 virus) of the same or different subtypes. Some such polypeptide variants induces in the subject to whom an effective amount of the polypeptide is administered antibodies capable of binding 2, 3, 4, 5, 6, 7, 8, 9, 10, 11, 12, 13, 14, 15, 16, 17, 18, 19, 20 or more HIV viruses (e.g., HIV-1 viruses) of the same or different subtypes or any combination thereof. Such subtypes include, but are not limited to, subtypes A, B, C, D, F, G, H, and J. Some such polypeptides induce the production of antibodies against at least 2-11 HIV-1 viruses selected from the group consisting of BAL, Bx08, QZ4589, 1196, JRCSF, 92HT594, 692, 93US073, NL4-3, JR-FL, and SF-162. Antibodies induced by such polypeptides may comprise neutralizing antibodies.

In another aspect, the invention provides an isolated or recombinant polypeptide variant comprising a polypeptide sequence having at least 90%, 91%, 92%, 93%, 94%, 95%, 96%, 97%, 98%, 99%, 99.5% or 100% sequence identity to any one of SEQ ID NOS:1-21 and 56-63, wherein said polypeptide variant induces in a subject to whom an effective amount of the polypeptide variant is administered production of antibodies against at least one HIV-1 pseudovirus, wherein each pseudovirus comprises a gp160 envelope protein of an HIV-1 virus. Each gp160 envelope polypeptide may be obtained from, derived from, or based on a same subtype (e.g., HIV-1 subtype A, B, C, D, F, G, H, or J) or a different subtype or any combination thereof. For example, the pseudovirus may comprise a gp160 envelope protein of an HIV-1 virus subtype B selected from the group consisting of BAL, Bx08, QZ4589, 1196, JRCSF, 92HT594, 692, 93US073, NL4-3, JR-FL and SF-162. The antibodies induced may be neutralizing antibodies against at least 1, 2, 3, 4, 5, 6, 7, 8, 9, 10, or 11 or more HIV-1 pseudoviruses, wherein each pseudovirus comprises a gp160 envelope polypeptide of a different HIV-1 pseudovirus.

In another aspect, the invention provides an isolated or recombinant polypeptide comprising a polypeptide sequence having at least 85%, 86%, 87%, 88%, 89%, 90%, 91%, 92%, 93%, 94%, 95%, 96%, 97%, 98%, 99%, 99.5% or 100% amino acid sequence identity to a polypeptide sequence selected from the group consisting of SEQ ID NOS:8-21, wherein the polypeptide induces in a subject to whom an effective amount of at least one such polypeptide is administered an immune response against at least one HIV (e.g., HIV-1) virus or pseudovirus. The amount of the polypeptide that is administered is an amount effective to induce a detectable immune response. Some such polypeptides induce in the subject an immune response against at least two or more HIV-1 viruses or pseudoviruses. The two or more HIV-1 viruses or pseudoviruses may be of the same HIV-1 virus or pseudovirus subtype (e.g., subtype B) or different subtypes, or any combination thereof. Such a polypeptide may induce in a subject to whom an effective amount of the polypeptide is administered an immune response that comprises an anti-HIV (e.g., HIV-1) neutralizing antibody response or HIV-specific (e.g., HIV-1-specific) T cell immune response or both. In one aspect, a cross reactive response against at least 1, 2, 3, 4, 5, 6, 7, 8, 9, 10, 11 or more different HIV-1 viruses or pseudoviruses is induced. Such cross reactive immune response includes, e.g., the production of neutralizing antibodies and/or a specific T cell response against two or more HIV-1 viruses or pseudoviruses of the same and/or different subtypes. For example, the induced neutralizing Ab response may be against at least 1, 2, 3, 4, 5, 6, 7, 8, 9, 10, or 11 HIV-1 viruses or pseudoviruses of the same or different subtypes or any combination of subtypes. Some such polypeptides induce in such subject the production of a titer of HIV neutralizing antibodies that is greater than the titer of HIV neutralizing antibodies induced in the subject by an HIV gp120 envelope polypeptide (e.g., HIV-1 JRCSF gp120 polypeptide). Some such polypeptides induce in such subject the production of antibodies capable of binding to at least one HIV-1 virus or HIV-1 pseudovirus.

In yet another aspect, the invention provides an isolated or recombinant polypeptide comprising a polypeptide sequence having at least 85%, 86%, 87%, 88%, 89%, 90%, 91%, 92%, 93%, 94%, 95%, 96%, 97%, 98%, 99%, 99.5% or 100% sequence identity to a sequence selected from the group consisting of SEQ ID NOS:1-7 and 56-63, wherein the polypeptide binds or specifically binds an HIV-1 neutralizing Ab. Some such polypeptides have a binding affinity for the HIV-1 neutralizing Ab that is about equal to or greater than the binding affinity of a WT HIV-1 gp120 full-length envelope polypeptide for the HIV-1 neutralizing Ab. HIV-1 neutralizing antibodies are known in the art and include, e.g., mAb IgG1b12 and 2G12. Some such polypeptides alternatively or in addition have a binding affinity for an HIV-1 non-neutralizing Ab that is lower than the binding affinity of a full-length WT HIV-1 gp120 envelope polypeptide for the HIV-1 non-neutralizing Ab. Some such gp120 full-length polypeptide variants have: (1) a binding affinity for an HIV-1 neutralizing Ab that is greater than the binding affinity of a WT HIV-1 JRCSF gp120 full-length envelope polypeptide for said HIV-1 neutralizing Ab; and/or (2) a binding affinity for an HIV-1 non-neutralizing Ab that is lower than the binding affinity of the WT HIV-1 JRCSF gp120 full-length Env polypeptide for the HIV-1 non-neutralizing Ab. HIV-1 non-neutralizing antibodies are known in the art and include, e.g., the mAbs b3 and b6. mAb b3 may comprise IgG b3 or Fab (antibody binding fragment) b3; b6 mAb may comprise IgG b6 or Fab (antibody binding fragment) b6. In some instances, such a polypeptide may exhibit a b12/b3 binding affinity ratio that is greater than the b12/b3 binding affinity ratio of a WT HIV-1 gp120 full-length Env polypeptide. In addition or alternatively, the polypeptide may exhibit a b12/b6 binding affinity ratio that is greater than the b12/b6 binding affinity ratio of a WT HIV-1 gp120 full-length Env polypeptide. An exemplary WT HIV-1 gp120 full-length Env polypeptide is HIV-1 JRCSF gp120 full-length Env polypeptide. Some such polypeptides may induce in a subject to whom an effective amount of at least one such polypeptide is administered the production of neutralizing antibodies against 1, 2, 3, 4, 5, 6, 7, 8, 9, 10 or more HIV-1 viruses of the same or different subtypes.

In another aspect, the invention provides an isolated or recombinant polypeptide comprising a polypeptide sequence having at least 85%, 86%, 87%, 88%, 89%, 90%, 91%, 92%, 93%, 94%, 95%, 96%, 97%, 98%, 99%, 99.5% or 100% sequence identity to a polypeptide sequence selected from the group consisting of SEQ ID NOS:8-21, wherein the polypeptide binds or specifically binds an HIV-1 neutralizing antibody. Some such polypeptides have a binding affinity for the HIV-1 neutralizing Ab that is about equal to or greater than the binding affinity of a WT HIV-1 gp120 core polypeptide (e.g., “core construct” designed based on a WT HIV-1 gp120 sequence as described elsewhere herein) for the HIV-1 neutralizing Ab (e.g., IgG1b12 or 2G12). Some such core polypeptides alternatively or in addition have a binding affinity for an HIV-1 non-neutralizing Ab that is lower than the binding affinity of a WT HIV-1 gp120 core polypeptide for the HIV-1 non-neutralizing Ab (e.g., IgG b6, Fab b6, IgG b3, or Fab b3). For example, in one aspect, a gp120 core polypeptide variant of the invention may have: (1) a binding affinity for an HIV-1 neutralizing antibody that is greater than the binding affinity of a WT HIV-1 JRCSF gp120 core polypeptide for said HIV-1 neutralizing Ab; and/or (2) a binding affinity for an HIV-1 non-neutralizing Ab that is lower than the binding affinity of the WT HIV-1 JRCSF gp120 core polypeptide for said HIV-1 non-neutralizing Ab. Some such polypeptides may exhibit a b12/b3 binding affinity ratio that is greater than the b12/b3 binding affinity ratio of a WT HIV-1 gp120 core polypeptide. In addition or alternatively, such polypeptide may have a b12/b6 binding affinity ratio greater than the b12/b6 binding affinity ratio of a WT HIV-1 gp120 core polypeptide (e.g., JRCSF gp120 core polypeptide). Some such polypeptides induce the production of neutralizing antibodies against two or more HIV-1 viruses of the same or different subtypes in a subject to whom an effective amount of at least one such polypeptide is administered.

Some such polypeptides of the invention that bind an HIV-neutralizing antibody induce the production of antibodies against at least one HIV virus in a subject to whom an effective amount of such polypeptide(s) is administered. In a particular aspect, neutralizing antibodies against at least one HIV virus are induced. In another aspect, neutralizing antibodies against two or more HIV-1 viruses are induced. The induced titer level may be greater than that induced by a WT gp120 polypeptide (e.g., JRCSF gp120).

In another aspect, the invention includes an isolated or recombinant polypeptide comprising a fragment of a gp120 variant polypeptide sequence, the gp120 variant polypeptide sequence having at least 85%, 90%, 91%, 92%, 93%, 94%, 95%, 96%, 97%, 98%, 99%, 99.5%, or 100% sequence identity to a sequence selected from the group consisting of SEQ ID NOS:1-7 and 56-63, wherein said fragment induces production of neutralizing antibodies against HIV-1 in a subject to whom an effective amount of the fragment is administered, and said fragment comprises at least those amino acid residues of the gp120 variant polypeptide sequence located at positions corresponding by reference to amino acid residues of regions C2, C3, V4, C4, and V5 of the HIV-1 gp120-HXB2 envelope protein sequence (SEQ ID NO:54) shown in FIGS. 10A-10F, wherein the amino acid residues of the fragment are numbered by reference to amino acid residues of the gp120-HXB2 envelope protein.

In one particular embodiment, the regions of such a polypeptide of the invention are covalently linked in the same order as regions C2, C3, V4, C4, and V5 of the HIV-1 gp120-HXB2 envelope protein shown in FIGS. 10A-10F. Usually, two adjacent regions are covalently linked using a peptide linker, such as, e.g., a tripeptide linker, using standard techniques known in the art. An exemplary peptide linker comprises amino acid residues Gly-Ala-Gly (i.e., GAG) or the like.

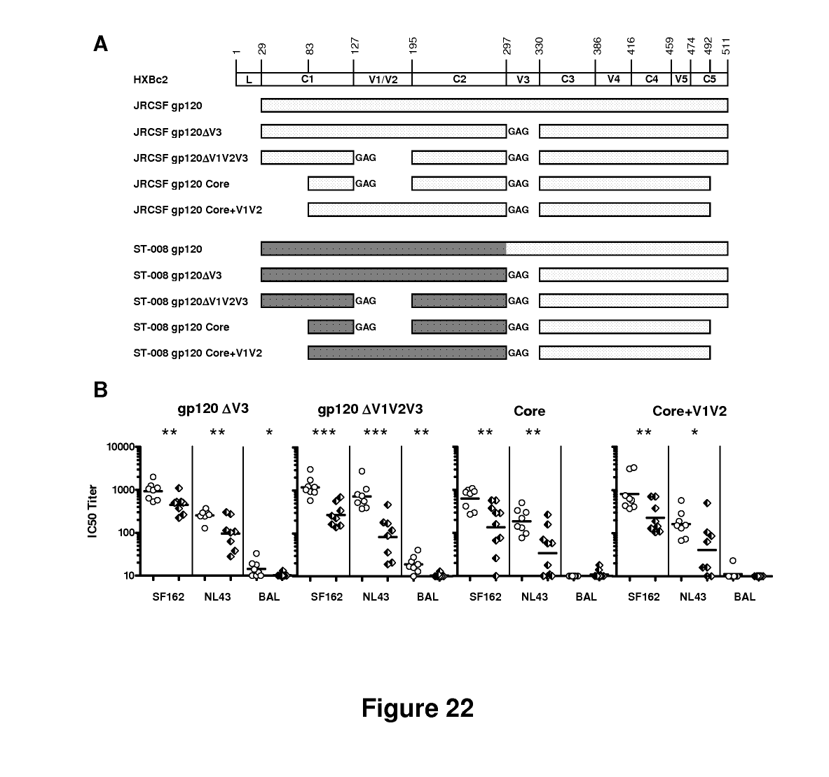

In a particular aspect, the fragment further comprises amino acid residues of the gp120 variant polypeptide sequence which correspond to amino acid residues 83-127 of the C1 region of the HXB2 gp120 envelope protein (SEQ ID NO:54) shown in FIGS. 10A-10F. In another aspect, the fragment further comprises amino acid residues of the gp120 variant polypeptide sequence which correspond to amino acid residues 472-492 of the C5 region of the HXB2 gp120 envelope protein (SEQ ID NO:54) shown in FIGS. 10A-10F. Exemplary fragment polypeptides according to this aspect include “gp120 Core”, “Core+V1V2”, “Core+V3” “gp120ΔV3” and “gp120ΔV1V2V3” polypeptides shown, for example, in FIGS. 22A, 23A, and 31A, such as, for example, fragments of the gp120 variant ST-008 (SEQ ID NO:1), said fragments having the sequences identified herein as SEQ ID NOS:107-110, each of which, as shown in Example 10 and FIGS. 22A-B and 23A-B, induces an immune response (e.g., a neutralizing antibody response) against more than one HIV-1 pseudovirus.

In another embodiment, the fragment further comprises amino acid residues of the gp120 variant polypeptide sequence which correspond to amino acid residues 29-82 of the C1 region of the HXB2 gp120 envelope protein (SEQ ID NO:54) shown in FIGS. 10A-10F. In another embodiment, the fragment further comprises amino acid residues of the gp120 variant polypeptide sequence which correspond to amino acid residues 493-511 of the C5 region of the HXB2 gp120 envelope protein (SEQ ID NO:54) shown in FIGS. 10A-10F. Exemplary polypeptides according to these embodiments include the “gp120ΔV3” and “gp120ΔV1V2V3” fragments described in Example 10 and FIGS. 22A-B and 23A-B, such as, for example, fragments of the gp120 variant ST-008 (SEQ ID NO:1), said fragments having the sequences identified herein as SEQ ID NOS:107-108, each of which, as shown in Example 10 and FIGS. 22A-B and 23A-B, induces an immune response (e.g., a neutralizing antibody response) against more than one HIV-1 pseudovirus.

In another aspect, the invention includes an isolated or recombinant polypeptide comprising a first, a second, a third, a fourth and a fifth subsequence of a gp120 variant sequence, the gp120 variant sequence comprising an amino acid sequence having at least 85%, 86%, 87%, 88%, 89%, 90%, 91%, 92%, 93%, 94%, 95%, 96%, 97%, 98%, 99%, 99.5% or 100% sequence identity to a polypeptide sequence selected from the group consisting of SEQ ID NOS:1-21 and 56-63, wherein: (a) the first subsequence of the gp120 variant sequence comprises a sequence corresponding by reference to amino acid residues 83-127 of the C1 region of the recombinant HXB2 gp120 envelope protein (SEQ ID NO:54) shown in FIGS. 10A-10F, wherein the C-terminus of the first subsequence is covalently linked by a peptide bond to the N-terminus of a first linker peptide; (b) the second subsequence of the gp120 variant sequence corresponds by reference to the C2 region of the recombinant HXB2 gp120 envelope protein (SEQ ID NO:54) shown in FIGS. 10A-10F, wherein the N-terminus of the second subsequence is covalently linked by a peptide bond to the C-terminus of the first linker peptide, and the C-terminus of the second subsequence is covalently linked by a peptide bond to the N-terminus of a second linker peptide or a gp120 V3 region sequence; (c) the third subsequence of the gp120 variant sequence corresponds by reference to the C3 region of the recombinant HXB2 gp120 envelope protein (SEQ ID NO:54) shown in FIGS. 10A-10F, and the N-terminus of the third subsequence is covalently linked by a peptide bond to the C-terminus of the second linker polypeptide or the gp120 V3 region sequence, and the C-terminus of the third subsequence is covalently linked by a peptide bond to the N-terminus of a gp120 V4 region sequence; (d) the fourth subsequence of the gp120 variant sequence corresponds by reference to the C4 region of the recombinant HXB2 gp120 envelope protein (SEQ ID NO:54) shown in FIGS. 10A-10F, and the N-terminus of the fourth subsequence is covalently linked by a peptide bond to the C-terminus of the gp120 V4 region sequence, and the C-terminus of the fourth subsequence is covalently linked by a peptide bond to the N-terminus of a gp120 V5 region sequence; and (e) the fifth subsequence of the gp120 variant sequence comprises a sequence corresponding by reference to amino acid residues 472-492 of the C5 region of the recombinant HXB2 gp120 envelope protein (SEQ ID NO:54) shown in FIGS. 10A-10F, and the N-terminus of the fifth subsequence is covalently linked by a peptide bond to the C-terminus of the V5 region sequence; wherein the gp120 V3 region sequence, the gp120 V4 region sequence and the gp120 V5 region sequence correspond by reference to the V3 region, the V4 region, and the V5 region, respectively, of the recombinant HXB2 gp120 envelope protein (SEQ ID NO:54) shown in FIGS. 10A-10F, and one or more of the gp120 V3 region sequence, the gp120 V4 region sequence and the gp120 V5 region sequence is not a subsequence of the selected gp120 variant sequence.

In one particular embodiment, one or more of the gp120 V3 region sequence, the gp120 V4 region sequence, and the gp120 V5 region sequence is a subsequence of (i) the amino acid sequence of a gp120 variant selected from the group consisting of SEQ ID NOS:1-21 and SEQ ID NOS:56-63 excluding the selected gp120 variant sequence, or (ii) the gp120 amino acid sequence of an HIV-1 viral strain, which subsequence corresponds by reference to the V3 region, the V4 region, or the V5 region, respectively, of the recombinant HXB2 gp120 envelope protein (SEQ ID NO:54) shown in FIGS. 10A-10F, wherein said one or more gp120 V3 region sequence, gp120 V4 region sequence, or gp120 V5 region sequence is not identical to the gp120 V3 region sequence, gp120 V4 region sequence, or gp120 V5 region sequence, respectively, of the selected gp120 variant sequence. The gp120 amino acid sequence of the HIV-1 strain may be the gp120 amino acid sequence of an HIV-1 subtype A strain, an HIV-1 subtype B strain, an HIV-1 subtype C strain, an HIV-1 subtype E strain, an HIV-1 subtype F strain, or an HIV-1 subtype G strain. The gp120 amino acid sequence of the HIV-1 strain may be the gp120 amino acid sequence of, for example, one of the HIV-1 strains listed in Table 10 herein.

In a particular aspect, the gp120 amino acid sequence of the HIV-1 viral strain may be the gp120 sequence of an HIV-1 subtype B strain, such as the gp120 sequence of JRCSF (SEQ ID NO:80), 89.6 (SEQ ID NO:81), 92HT593 (SEQ ID NO:82), 92HT594 (SEQ ID NO:83), 92HT596 (SEQ ID NO:84), 92HT599 (SEQ ID NO:85), 92US657 (SEQ ID NO:86), 92US712 (SEQ ID NO:87), 92US727 (SEQ ID NO:88), or 93US073 (SEQ ID NO:89).

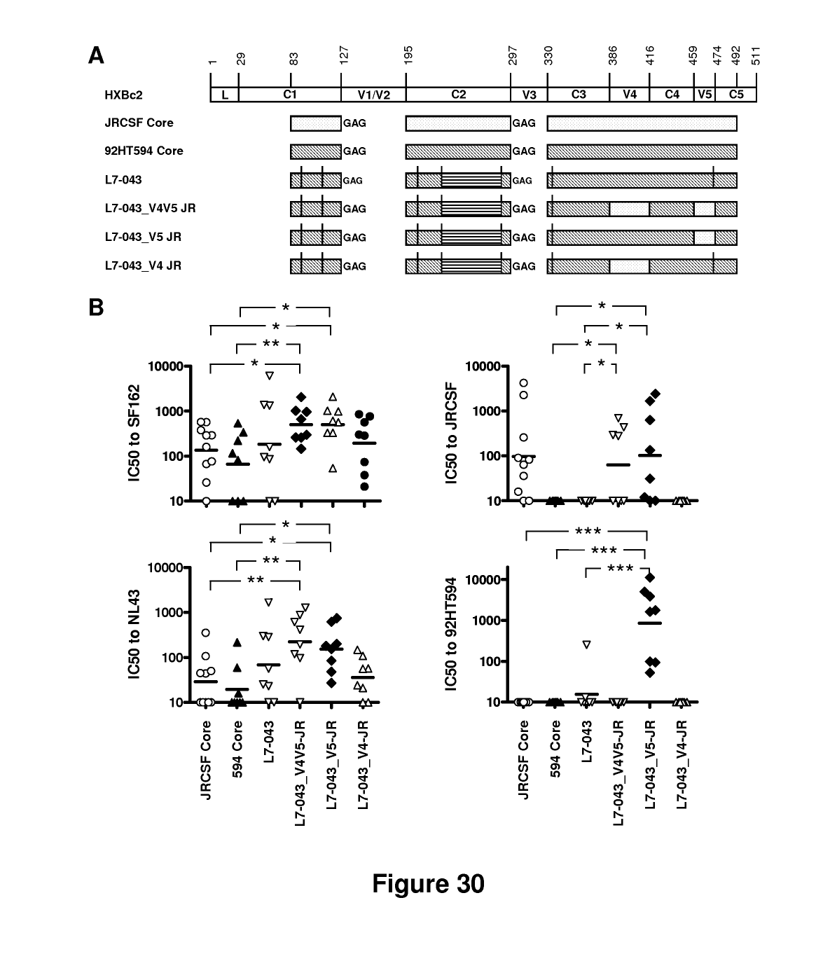

Exemplary polypeptides according to this aspect of the invention include polypeptides identified herein as L7-043_V4V5 JR (SEQ ID NO:131), L7-043_V4 JR (SEQ ID NO:132), L7-043_V5 JR (SEQ ID NO:133) and L7-043 Core+V3 JR (SEQ ID NO:134), in which the selected amino acid sequence is the sequence of variant L7-043 (SEQ ID NO:10) and one or more of the gp120 V3 domain sequence, the gp120 V4 domain sequence, and the gp120 V5 domain sequence is a subsequence of the gp120 amino acid sequence of the HIV-1 viral strain JRCSF. As shown in Example 13 and FIGS. 30A-B and 31A-B, each of these exemplary polypeptides according to this aspect of the invention induces an immune response (e.g., a neutralizing antibody response) against more than one HIV-1 pseudovirus.

In yet another embodiment, two or three of the gp120 V3 domain sequence, the gp120 V4 domain sequence, and the gp120 V5 domain sequence are subsequences of gp120 amino acid sequences of different HIV-1 viral strains, such as different HIV-1 subtype strains, e.g., different HIV-1 subtype B strains, such as one of the HIV-1 subtype B strains specified above.

In yet another aspect, exemplary polypeptides of the invention include chimeric gp120 polypeptide variants comprising the amino acid sequences identified herein as SEQ ID NOS:1-7, such as SEQ ID NO:1, SEQ ID NO:2, SEQ ID NO:3, SEQ ID NO:4, SEQ ID NO:5, SEQ ID NO:6, and SEQ ID NO:7, which are encoded by nucleic acid sequences identified herein as SEQ ID NOS:23-29 or SEQ ID NOS:37-43 (codon-optimized for expression in mammalian cells), respectively. Exemplary polypeptides of the invention also include chimeric gp120 polypeptide variants comprising the amino acid sequences identified herein as SEQ ID NOS:56-63, which are encoded by nucleic acid sequences identified herein as SEQ ID NOS:64-71 or SEQ ID NOS:72-79 (codon-optimized for expression in mammalian cells), respectively. Some such polypeptides have a b12/b3 binding affinity ratio that is equal to or greater than the b12/b3 binding affinity ratio of an HIV-1 gp120 polypeptide (e.g., HIV-1 JRCSF gp120 (SEQ ID NO: 80)) and/or have a b12/b6 binding affinity ratio that is equal to or greater than the b12/b6 binding affinity ratio of an HIV-1 gp120 polypeptide (e.g., HIV-1 JRCSF gp120 (SEQ ID NO:80)). Some such polypeptides induce in a subject to whom an effective amount of such polypeptide(s) is administered at least one immune response as discussed above against HIV-1, such as a humoral (e.g., antibody) or cell-mediated (e.g., T cell lymphocyte) immune response.

As discussed in detail below, the invention also includes an isolated or recombinant nucleic acid that encodes any one of the polypeptide sequences of SEQ ID NOS:1-7 and 56-63, including, e.g., a nucleotide sequence that has been optimized for expression in mammals, such as primates, including humans, as discussed in greater detail infra. Optimization can be achieved by, e.g., selecting codons that are substantially expressed in mammalian cells of interest. Exemplary nucleic acids which have been optimized for expression in mammalian cells and which encode the sequences of SEQ ID NOS:1-7 include those nucleotide sequences identified by SEQ ID NOS:37-43, respectively. Exemplary nucleic acids which have been optimized for expression in mammalian cells and which encode the sequences of SEQ ID NOS:56-63 include those identified by SEQ ID NOS:72-79, respectively.

The invention also includes a recombinant or isolated chimeric polypeptide variant comprising an amino acid sequence which differs from the sequence set forth in any one of SEQ ID NOS:1-21 and 56-63 by 1 to 60 amino acid residues (such as by 1, 2, 3, 4, 5, 6, 7, 8, 9, 10, 11, 12, 13, 14, 15, 16, 17, 18, 19, 20, etc. amino acid residues) and which exhibits the ability to induce an immune response against at least one HIV virus, e.g., HIV-1. The immune response may be humoral or cellular response against one or more HIV-1 strains of the same subtype or different subtypes. The immune response may comprise a neutralizing antibody response and/or specific T cell response against at least one HIV virus (e.g., HIV-1) or both. Such polypeptide variant may be capable of binding a known HIV-1 neutralizing mAb. Such polypeptide variant may induce in a subject to whom an effective amount of the variant is administered the production of a titer of HIV neutralizing antibodies that is greater than the titer of HIV neutralizing antibodies induced in the subject by administration of an equal amount of an HIV gp120 Env polypeptide (e.g., HIV-1 JRCSF gp120 polypeptide). Such polypeptide variant may further comprise an additional amino acid, such as a methionine, added to the N-terminus, and/or a signal peptide and/or may include an immunoglobulin (Ig) domain or portion, thereby forming a fusion protein.

Polypeptides of the invention, including full-length gp120 polypeptides, core gp120 polypeptides, core+V1V2 polypeptides, core+V3 polypeptides, gp120ΔV3 polypeptides and gp120ΔV1V2V3 of the invention, including those discussed above (such as, e.g., those represented by SEQ ID NOS:1-21, 56-63, 107-110 and 131-134), optionally further comprise a signal peptide, such as, e.g., but not limited to, the signal peptide comprising the sequence of SEQ ID NO:52 (tissue plasminogen activator signal peptide) or SEQ ID NO:55 (signal peptide of the HIV-1 HXB2 gp120 full-length polypeptide). See Golden, A. et al., Protein Expression and Purification 14:8-12 (1998); Pennica, D. et al., Nature 301:214-221 (1983). A variety of known signal peptides can be used, including the signal peptide sequences of wild-type HIV-1 gp120 polypeptides.

Polypeptides of the invention, including those discussed above, optionally further comprise an additional amino acid, such as a methionine, added to the N-terminus and/or a peptide tag for purification or identification. Polypeptides of the invention, including those discussed above, optionally further comprise a fusion protein comprising at least one additional amino acid sequence. For example, polypeptides of the invention may comprise a polypeptide purification subsequence, such as, e.g., a subsequence is selected from an epitope tag, a FLAG tag, a polyhistidine sequence, and a GST fusion.

The invention also provides fusion proteins and conjugates comprising these polypeptides. In addition, as discussed in greater detail below, the invention includes isolated, recombinant, or synthetic nucleic acids encoding all polypeptides of the invention. The invention includes a nucleic acid that encodes a protein comprising a signal peptide and a polypeptide of the invention that induces an immune response against HIV or binds to an HIV neutralizing antibody. The encoded signal peptide sequence, which directs secretion of the mature polypeptide through a prokaryotic or eukaryotic cell membrane, is typically covalently linked to the N-terminus of said polypeptide. A variety of signal peptides can be used, including the sequence set forth in SEQ ID NO:52, which is encoded by, e.g., the nucleotide sequence shown in SEQ ID NO:53.

A recombinant or isolated polypeptide of the invention (including, e.g., a chimeric HIV-1 gp120 full-length polypeptide variant, core gp120 polypeptide variant, core+V1V2 polypeptide variant, core+V3 polypeptide variant, gp120ΔV3 polypeptide variant or gp120ΔV1V2V3 polypeptide variant) displays HIV antigen specificity, e.g., if it produces a positive reaction with an antibody to an HIV antigen in any immunoassays disclosed herein or known in the art. A polypeptide of the invention (including, e.g., a chimeric HIV-1 gp120 full-length polypeptide variant, core gp120 polypeptide variant, core+V1V2 polypeptide variant, core+V3 polypeptide variant, gp120ΔV3 polypeptide variant or gp120ΔV1V2V3 polypeptide variant) displays HIV antigenicity, e.g., if it elicits diagnostically useful antibodies for use in the detection of HIV infection in any such immunoassay. A population of recombinant or synthetic polypeptides of the invention (e.g., produced by shuffling various DNA sequences encoding various parental HIV-1 gp120 Env protein antigens) can be screened for a recombinant or synthetic polypeptide that exhibits an antigenicity that is qualitatively and/or quantitatively different from the antigenicity of an HIV-1 gp120 Env protein antigen or other HIV antigen using a standard assay, such as, e.g., ELISA or Ouchterlony immunodiffusion assays (J. M. BREWER ET AL., EXPERIMENTAL TECHNIQUES IN BIOCHEMISTRY 116-121 (1974). For example, such a population can be screened for a recombinant or synthetic polypeptide(s) that induces a greater immune response than that induced by an HIV-1 gp120 Env protein. An ELISA assay is typically used to determine whether two polypeptides induce a quantitatively and/or qualitatively different antibody response in a host.

A population of recombinant polypeptide variants of the invention (e.g., produced by shuffling various DNA sequences encoding various parental HIV-1 gp120 Env protein antigens) can be screened for at least one recombinant polypeptide exhibiting an antigen specificity that differs from the antigen specificity of an HIV antigen, such as HIV-1 gp120 Env protein, using a standard assay, such as, e.g., an Ouchterlony immunodiffusion, ELISA, or RIA assay. Such population of variants can be screened for a recombinant polypeptide variant(s) that exhibits a specificity for antibodies against HIV-1 that is different from the specificity of HIV-1 gp120 Env polypeptide to those same anti-HIV-1 antibodies in Ouchterlony immunodiffusion, ELISA and/or RIA assays.

The Ouchterlony method is frequently used in comparisons of the antigenicity of a series of proteins to see how closely related such proteins are. The Ouchterlony technique can also be used to distinguish between the specificities of two polypeptides for an antibody to wild-type virus (i.e., whether the “highly specific fashion” in which each polypeptide reacts with an antibody to the WT virus differs). This assay can assist in determining whether: 1) the two antigens have no antigenic sites in common; 2) the two antigens have all antigenic sites in common; 3) all of the antigenic sites for the antibody in the first antigen are also present in the second antigen, but the second antigen has antigenic sites that are not present in the first antigen; and/or 4) the two antigens have some sites in common and some not in common. See, e.g., J. M. BREWER ET AL., EXPERIMENTAL TECHNIQUES IN BIOCHEMISTRY 116-121 (1974).

Polypeptides of the invention, including those discussed above (e.g., those represented by polypeptide sequences having at least 90% identity to any one of SEQ ID NOS:1-21, 56-63, 107-110 and 131-134) that induce HIV-specific cellular and/or humoral immune responses are useful in methods of inducing and/or enhancing an immune response in a subject against one or more HIV-1 viruses and are believed useful as a vaccine to inhibit or protect against HIV-1 infection in mammals. Polypeptides of the invention, including those discussed above (e.g., those represented by polypeptide sequences having at least 90% identity to any one of SEQ ID NOS:1-21, 56-63, 107-110 and 131-134) that bind or react with HIV-1 mAbs are useful in diagnostic methods, including methods of determining whether the serum of a subject comprises monoclonal antibodies against HIV-1. These and other aspects of the invention are discussed in greater detail below.

Sequence Identity

As noted above, the invention provides an isolated or recombinant polypeptide which comprises a polypeptide sequence having at least 85%, 86%, 87%, 88%, 89%, 90%, 91%, 92%, 93%, 94%, 95%, 96%, 97%, 98%, 99%, 99.5%, or 100% sequence identity to a polypeptide sequence selected from the group consisting of SEQ ID NOS:1-21 and 56-63, and which induces an immune response against at least one HIV virus or pseudovirus (e.g., HIV-1), such as, e.g., an HIV-1 neutralizing antibody response. Some such polypeptides bind with one or more anti-HIV mAbs to different degrees.

The degree to which a sequence (polypeptide or nucleic acid) is similar to another provides an indication of similar structural and functional properties for the two sequences. Accordingly, in the context of the present invention, sequences that have a similar sequence to any given exemplar sequence are a feature of the present invention. Sequences that have percent sequence identities as defined below are a feature of the invention.

A variety of methods of determining sequence relationships can be used, including manual alignment and computer assisted sequence alignment and analysis. A variety of computer programs for performing sequence alignment are available, or can be produced by one of skill

The sequences of the nucleic acids and polypeptides employed in the subject invention need not be identical, but can be substantially identical to the corresponding sequence of a polypeptide of the invention or nucleic acid of the invention. For example, polypeptides of the invention can be subject to various changes, such as one or more amino acid insertions, deletions, and/or substitutions, either conservative or non-conservative, including where, e.g., such changes might provide for certain advantages in their use, such as, in their therapeutic or prophylactic use or administration or diagnostic application. The nucleic acids of the invention can also be subject to various changes, such as one or more substitutions of one or more nucleic acids in one or more codons such that a particular codon encodes the same or a different amino acid, resulting in either a silent variation (as defined herein) or non-silent variation, or one or more deletions of one or more nucleic acids (or codons) in the sequence. The nucleic acids can also be modified to include one or more codons that provide for optimum expression in an expression system (e.g., bacterial or mammalian), while, if desired, said one or more codons still encode the same amino acid(s). Such nucleic acid changes might provide for certain advantages in their therapeutic or prophylactic use or administration, or diagnostic application. The nucleic acids and polypeptides can be modified in a number of ways so long as they comprise a sequence substantially identical (as defined below) to a sequence in a respective nucleic acid or polypeptide of the invention.

The term “identical” or “identity,” in the context of two or more nucleic acid or polypeptide sequences, refers to two or more sequences that are the same or have a specified percentage of amino acid residues or nucleotides that are the same, when compared and aligned for maximum similarity, as determined using the sequence comparison algorithm described below or by visual inspection.

The “percent sequence identity” (“% identity”) of a subject sequence to a reference (i.e. query) sequence means that the subject sequence is identical (i.e., on an amino acid-by-amino acid basis for a polypeptide sequence, or a nucleotide-by-nucleotide basis for a polynucleotide sequence) by a specified percentage to the query sequence over a comparison length.

The percent sequence identity of a subject sequence to a query sequence can be calculated as follows. First, the optimal alignment of the two sequences is determined using a sequence comparison algorithm with specific alignment parameters. This determination of the optimal alignment may be performed using a computer, or may be manually calculated, as described below. Then, the two optimally aligned sequences are compared over the comparison length, and the number of positions in the optimal alignment at which identical residues occur in both sequences are determined, which provides the number of matched positions. The number of matched positions is then divided by the total number of positions of the comparison length (which, unless otherwise specified, is the length of the query sequence), and then multiplying the result by 100, to yield the percent sequence identity of the subject sequence to the query sequence.

With regard to polypeptide sequences, typically one sequence is regarded as a “query sequence” (for example, a polypeptide sequence of the invention) to which one or more other sequences, i.e., “subject sequence(s)” (for example, sequences present in a sequence database) are compared. The sequence comparison algorithm uses the designated alignment parameters to determine the optimal alignment between the query sequence and the subject sequence(s). When comparing a query sequence against a sequence database, such as, e.g., GENBANK® (Genetic Sequence Data Bank; U.S. Department of Health and Human Services) or GENESEQ® (Thomson Derwent; also available as DGENE® on STN), usually only the query sequence and the alignment parameters are input into the computer; optimal alignments between the query sequence and each subject sequence are returned for up to a specified number of subject sequences.

Two polypeptide sequences are “optimally aligned” when they are aligned using defined parameters, i.e., a defined amino acid substitution matrix, gap existence penalty (also termed gap open penalty), and gap extension penalty, so as to arrive at the highest similarity score possible for that pair of sequences. The BLOSUM62 matrix (Henikoff and Henikoff Proc. Natl. Acad. Sci. USA 89(22):10915-10919 (1992)) is often used as a default scoring substitution matrix in polypeptide sequence alignment algorithms (such as BLASTP, described below). The gap existence penalty is imposed for the introduction of a single amino acid gap in one of the aligned sequences, and the gap extension penalty is imposed for each residue position in the gap. Unless otherwise stated, alignment parameters employed herein are: BLOSUM62 scoring matrix, gap existence penalty=11, and gap extension penalty=1. The alignment score is defined by the amino acid positions of each sequence at which the alignment begins and ends (e.g. the alignment window), and optionally by the insertion of a gap or multiple gaps into one or both sequences, so as to arrive at the highest possible similarity score.

While optimal alignment between two or more sequences can be determined manually (as described below), the process is facilitated by the use of a computer-implemented alignment algorithm such as BLAST® (National Library of Medicine), e.g., BLASTP for polypeptide sequences and BLASTN for nucleic acid sequences, described in Altschul et al. (1997) Nucleic Acids Res. 25:3389-3402, and made available to the public through various sources, such as the National Center for Biotechnology Information (NCBI) Website. When using a computerized BLAST interface, if the option exists to use a “low complexity filter”, this option should be turned off (i.e., no filter).

Optimal alignment between two polypeptide sequences can also be determined by a manual calculation of the BLASTP algorithm (i.e., without aid of a computer) using the same alignment parameters specified above (matrix=BLOSUM62, gap open penalty=11, and gap extension penalty=1). To begin, the two sequences are initially aligned by visual inspection. An initial alignment score is then calculated as follows: for each individual position of the alignment (i.e., for each pair of aligned residues), a numerical value is assigned according to the BLOSUM62 matrix. The sum of the values assigned to each pair of residues in the alignment is the initial alignment score. If the two sequences being aligned are highly similar, often this initial alignment provides the highest possible alignment score. The alignment with the highest possible alignment score is the optimal alignment based on the alignment parameters employed. For example, to calculate an alignment score for two sequences (a “query” sequence and a “subject” sequence), the sequences can be aligned by visual inspection, and a numerical value is assigned by the BLOSUM62 matrix for each aligned pair of amino acids. An optimal alignment is one providing the highest possible alignment score (the sum of the values for each aligned position) can be determined; any other alignment of these two sequences, with or without gaps, would result in a lower alignment score.

In some instances, a higher alignment score might be obtained by introducing one or more gaps into the alignment. Whenever a gap is introduced into an alignment, a gap open penalty is assigned, and in addition a gap extension penalty is assessed for each residue position within that gap. Therefore, using the alignment parameters described above (including gap open penalty=11 and gap extension penalty=1), a gap of one residue in the alignment would correspond to a value of −(11+(1×1))=−12 assigned to the gap; a gap of three residues would correspond to a value of −(11+(3×1))=−14 assigned to the gap, and so on. This calculation is repeated for each gap introduced into the alignment. Introduction of a gap into an alignment can result in a higher alignment score, despite the gap penalty.

It is to be understood that the examples of sequence alignment calculations described above, which use relatively short sequences, are provided for illustrative purposes only; in practice, the alignment parameters employed (BLOSUM62 matrix, gap open penalty=11, and gap extension penalty=1) are optimal for polypeptide sequences of 85 amino acids or longer. The NCBI website provides the following alignment parameters for sequences of other lengths (which are suitable for computer-aided as well as manual alignment calculation, using the same procedure as described above). For sequences of 50-85 amino acids in length, optimal parameters are BLOSUM80 matrix (Henikoff and Henikoff, supra), gap open penalty=10, and gap extension penalty=1. For sequences of 35-50 amino acids in length, optimal parameters are PAM70 matrix (Dayhoff, M. O., Schwartz, R. M. & Orcutt, B. C. (1978) “A model of evolutionary change in proteins.” In Atlas of Protein Sequence and Structure, vol. 5, suppl. 3, M. O. Dayhoff (ed.), pp. 345-352, Natl. Biomed. Res. Found., Washington, D.C.), gap open penalty=10, and gap extension penalty=1. For sequences of less than 35 amino acids in length, optimal parameters are PAM30 matrix (Dayhoff, M. O., supra), gap open penalty=9, and gap extension penalty=1.

Once the sequences are optimally aligned, the percent identity of the subject sequence relative to the query sequence is calculated by counting the number of positions in the optimal alignment which contain identical residue pairs, divide that by the number of residues in the comparison length (which, unless otherwise specified, is the number of residues in the query sequence), and multiplying the resulting number by 100.

An optimal alignment typically is one that provides the highest level of identity between the aligned sequences. In some formats for obtaining an optimal alignment, gaps can be introduced, and some amount of non-identical sequences and/or ambiguous sequences can be ignored to obtain an alignment that provides the highest level of identity between the aligned sequences. The introduction of gaps and/or the ignoring of non-homologous/ambiguous sequences are often associated with a gap penalty, unless otherwise stated herein. In other words, a gap between two sequences will reduce the level of identity by one residue or nucleotide base.

As noted above, alignment and comparison of relatively short sequences (less than about 30 residues) is typically straightforward, and identity between relatively short amino acid or nucleic acid sequences can be easily determined by visual inspection. Comparison of longer sequences can require more sophisticated methods to achieve optimal alignment of two sequences. Analysis with an appropriate algorithm, such as those discussed above, typically facilitated through computer software, commonly is used to determine identity between longer sequences. When using a sequence comparison algorithm, test and reference sequences typically are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. A number of mathematical algorithms for obtaining the optimal alignment and calculating identity between two or more sequences are known and incorporated into a number of available software programs. Examples of such programs include the MATCH-BOX, MULTAIN, GCG, FASTA, and ROBUST programs for amino acid sequence analysis, and the SIM, GAP, NAP, LAP2, GAP2, and PIPMAKER programs for nucleotide sequences. Suitable software analysis programs for both amino acid and polynucleotide sequence analysis include the ALIGN, CLUSTALW (e.g., version 1.6 and later versions thereof, such as version W 1.8 available from European Bioinformatics Institute, Cambridge, UK), and BLAST programs (e.g., BLAST 2.1, BL2SEQ, and later versions thereof). Select examples are further described in the following paragraphs.

For amino acid sequence analysis and amino acid alignments, a weight matrix, such as the BLOSUM matrixes (e.g., the BLOSUM45, BLOSUM50, BLOSUM62, and BLOSUM80 matrixes—as described in, e.g., Henikoff and Henikoff, Proc. Natl. Acad. Sci. USA 89:10915-10919 (1992)), Gonnet matrixes (e.g., the Gonnet40, Gonnet80, Gonnet120, Gonnet160, Gonnet250, and Gonnet350 matrixes), or PAM matrixes (e.g., the PAM30, PAM70, PAM120, PAM160, PAM250, and PAM350 matrixes), are used in determining identity. BLOSUM matrixes, such as the BLOSUM50 and BLOSUM62 matrixes are commonly used. In the absence of availability of such weight matrixes (e.g., in nucleic acid sequence analysis and with some amino acid analysis programs), a scoring pattern for residue/nucleotide matches and mismatches can be used (e.g., a +5 for a match and −4 for a mismatch pattern).

The ALIGN program produces an optimal global (overall) alignment of the two chosen protein or nucleic acid sequences using a modification of the dynamic programming algorithm described by Myers and Miller CABIOS 4:11-17 (1988). The ALIGN program typically, although not necessary, is used with weighted end-gaps. If gap opening and gap extension penalties are available, they are often set between about −5 to −15 and 0 to −3, respectively, more preferably about −12 and −0.5 to −2, respectively, for amino acid sequence alignments, and −10 to −20 and −3 to −5, respectively, more commonly about −16 and −4, respectively, for nucleic acid sequence alignments. The ALIGN program and principles underlying it are further described in, e.g., Pearson et al., Proc. Natl. Acad. Sci. USA 85:2444-48 (1988), and Pearson et al., Meth. Enzymol. 18:63-98 (1990).

Alternatively, and particularly for multiple sequence analysis (i.e., comparison of more than three sequences), the CLUSTALW program (described in, e.g., Thompson et al., Nucl. Acids Res. 22:4673-4680 (1994)) can be used. CLUSTALW is an algorithm suitable for multiple DNA and amino acid sequence alignments is the CLUSTALW program (Thompson et al., Nucl. Acids Res. 22:4673-4680 (1994)). CLUSTALW performs multiple pairwise comparisons between groups of sequences and assembles them into a multiple alignment based on homology. In one aspect, Gap open and Gap extension penalties are set at 10 and 0.05, respectively. Alternatively or additionally, the CLUSTALW program is run using “dynamic” (versus “fast”) settings. Typically, nucleotide sequence analysis with CLUSTALW is performed using the BESTFIT matrix, whereas amino acid sequences are evaluated using a variable set of BLOSUM matrixes depending on the level of identity between the sequences (e.g., as used by the CLUSTALW version 1.6 program available through the San Diego Supercomputer Center (SDSC) or version W 1.8 available from European Bioinformatics Institute, Cambridge, UK). Preferably, the CLUSTALW settings are set to the SDSC CLUSTALW default settings (e.g., with respect to special hydrophilic gap penalties in amino acid sequence analysis). The CLUSTALW program and underlying principles of operation are further described in, e.g., Higgins et al., CABIOS 8(2):189-91 (1992), Thompson et al., Nucleic Acids Res. 22:4673-80 (1994), and Jeanmougin et al., Trends Biochem. Sci. 2:403-07 (1998).

In an alternative format, the identity or percent identity between a particular pair of aligned amino acid sequences refers to the percent amino acid sequence identity that is obtained by CLUSTALW analysis (e.g., version W 1.8)), counting the number of identical matches in the alignment and dividing such number of identical matches by the greater of (i) the length of the aligned sequences, and (ii) 96, and using the following default ClustalW parameters to achieve slow/accurate pairwise alignments—Gap Open Penalty: 10; Gap Extension Penalty: 0.10; Protein weight matrix: Gonnet series; DNA weight matrix: IUB; Toggle Slow/Fast pairwise alignments=SLOW or FULL Alignment.

The polypeptide alignment shown in FIGS. 10A-10F was generated using the CLUSTALW algorithm (Thompson et al., Nucl. Acids Res. 22:4673-4680 (1994)) using for a pairwise alignment a gap opening penalty of 10 and a gap extension penalty of 0.1, using for the multiple (sequence) alignment a gap opening penalty of 10 and a gap extension penalty of 0.05, and the BLOSUM62 scoring substitution matrix (Henikoff and Henikoff, supra).

Another useful algorithm for determining percent identity or percent similarity is the FASTA algorithm, which is described in Pearson et al., Proc Natl. Acad. Sci. USA 85:2444 (1988). See also, Pearson, Methods Enzymol. 266:227-258 (1996). Typical parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Other suitable algorithms include the BLAST and BLAST 2.0 algorithms, which facilitate analysis of at least two amino acid or nucleotide sequences, by aligning a selected sequence against multiple sequences in a database (e.g., GeneSeq), or, when modified by an additional algorithm such as BL2SEQ, between two selected sequences. Software for performing BLAST analyses is publicly available through the National Center for Biotechnology Information (NCBI) (world wide website address ncbi.nlm.nih.gov). The BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) can be used with a word length (W) of 11, an expectation (E) of 10, M=5, N=−4 and a comparison of both strands. For amino acid sequences, the BLASTP program (e.g., BLASTP 2.0.14; Jun. 29, 2000) can be used with a word length of 3 and an expectation (E) of 10. The BLOSUM62 scoring matrix (see Henikoff & Henikoff, (1989) Proc. Natl. Acad. Sci. USA 89:10915) uses alignments (B) of 50, expectation (E) of 10, M=5, N=−4, and a comparison of both strands. Again, as with other suitable algorithms, the stringency of comparison can be increased until the program identifies only sequences that are more closely related to those in the sequence listing herein (e.g., a polypeptide comprising a polypeptide sequence having at least 85, 90, 91, 92, 93, 49, 95, 96, 97, 98, 99%, or 100% identity to a polypeptide sequence selected from SEQ ID NOS:1-21 and 56-63; or nucleic acid comprising a nucleotide sequence having at least 85, 90, 91, 92, 93, 49, 95, 96, 97, 98, 99%, or 100% identity to a nucleotide sequence selected from any of SEQ ID NOS:23-50 and 64-79.

The BLAST algorithm also performs a statistical analysis of the similarity or identity between two sequences (see, e.g., Karlin & Altschul, (1993) Proc. Natl. Acad. Sci. USA 90:5873-5787). One measure of similarity or identity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, such as less than about 0.01 or less than about 0.001.

BLAST program analysis also or alternatively can be modified by low complexity filtering programs such as the DUST or SEG programs, which are preferably integrated into the BLAST program operations (see, e.g., Wootton et al., Comput. Chem. 17:149-63 (1993), Altschul et al., Nat. Genet. 6:119-29 (1991), Hancock et al., Comput. Appl. Biosci. 10:67-70 (1991), and Wootton et al., Meth. Enzymol. 266:554-71 (1996)). In such aspects, if a lambda ratio is used, useful settings for the ratio are between 0.75 and 0.95, including between 0.8 and 0.9. If gap existence costs (or gap scores) are used in such aspects, the gap existence cost typically is set between about −5 and −15, more typically about −10, and the per residue gap cost typically is set between about 0 to −5, such as between 0 and −3 (e.g., −0.5). Similar gap parameters can be used with other programs as appropriate. The BLAST programs and principles underlying them are further described in, e.g., Altschul et al., J. Mol. Biol. 215:403-10 (1990), Karlin and Altschul, Proc. Natl. Acad. Sci. USA 87:2264-68 (199) (as modified by Karlin and Altschul, Proc. Natl. Acad. Sci. USA 90:5873-77 (1993)), and Altschul et al., Nucl. Acids Res. 25:3389-3402 (1997).

Another example of a useful algorithm is incorporated in PILEUP software. The PILEUP program creates a multiple sequence alignment from a group of related sequences using progressive, pair-wise alignments to show relationship and percent sequence identity or percent sequence similarity. PILEUP uses a simplification of the progressive alignment method of Feng & Doolittle (1987) J. Mol. Evol. 35:351-360, which is similar to the method described by Higgins & Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using specified parameters. Exemplary parameters for the PILEUP program are: default gap weight (3.00), default gap length weight (0.10), and weighted end gaps. PILEUP is a component of the GCG sequence analysis software package, e.g., version 7.0 (see, e.g., Devereaux et al. (1984) Nucl. Acids Res. 12:387-395).

Other useful algorithms for performing identity analysis include the local homology algorithm of Smith and Waterman (1981) Adv. Appl. Math. 2:482, the homology alignment algorithm of Needleman and Wunsch (1970) J. Mol. Biol. 48:443, and the search for similarity method of Pearson and Lipman (1988) Proc. Natl. Acad. Sci. USA 85:2444. Computerized implementations of these algorithms (e.g., GAP, BESTFIT, FASTA and TFASTA) are provided in the Wisconsin Genetics Software Package Release 7.0, (Genetics Computer Group, Madison, Wis.).

Several additional commercially available software suites incorporate the ALIGN, BLAST, and CLUSTALW programs and similar functions, and may include significant improvements in settings and analysis. Examples of such programs include the GCG suite of programs and those available through DNASTAR, Inc. (Madison, Wis.), such as the Lasergene® and Protean® programs. One alignment method is the Jotun Hein method, incorporated within the MegaLine™ DNASTAR package (MegaLine™ Version 4.03) used according to the manufacturer's instructions and default values specified in the program.

As applied to polypeptides, the term “substantial identity” (or “substantially identical”) typically means that when two amino acid sequences (i.e. a query sequence and a subject sequence) are optimally aligned using the BLASTP algorithm (manually or via computer) using appropriate parameters described above, the subject sequence has at least 60%, 70%, 75%, 80%, 85%, 90%, 91%, 92%, 93%, 94%, 95%, 96%, 97%, 98%, 99%, or 100% amino acid sequence identity to the query sequence. In some instances, the substantial identity exists over a comparison length of at least 100 amino acid residues, such as at least 110, 120, 125, 130, 135, 140, 145, 150, 200, 250, 300, 350, 400, 450, 500, or 525 amino acid residues.

Similarly, as applied in the context of two nucleic acid sequences, the term substantial identity (or substantially identical) means that when two nucleic acid sequences (i.e. a query and a subject sequence) are optimally aligned using the BLASTN algorithm (manually or via computer) using appropriate parameters described below, the subject sequence has at least about 60%, 70%, 75%, 80%, 85%, 90%, 91%, 92%, 93%, 94%, 95%, 96%, 97%, 98%, 99%, or 100% nucleic acid sequence identity to the query sequence. Parameters used for nucleic acid sequence alignments are: match reward 1, mismatch penalty −3, gap existence penalty 5, gap extension penalty 2 (substitution matrices are not used in the BLASTN algorithm). In some instances, the substantial identity exists over a comparison length of at least 300 nucleotide residues, such as at least 330, 360, 375, 390, 405, 420, 435, 450, 600, 750, 900, 1050, 1200, 1350, 1500, or 1575 nucleotide residues.

Sequence Variation

The invention includes polypeptides that comprise conservatively modified variations of any polypeptide sequence of the invention described herein. In a particular aspect, such polypeptide variants include conservatively modified variations of a polypeptide sequence selected from the group of SEQ ID NOS:1-21, 56-63, 107-110 and 131-134.

A conservative amino acid residue substitution typically involves exchanging a member within one functional class of amino acid residues for a residue that belongs to the same functional class (identical amino acid residues are considered functionally homologous or conserved in calculating percent functional homology).

Conservative substitution tables providing functionally similar amino acids are well known in the art. Table 1 sets forth exemplary functional classes of amino acids and members of those classes that would constitute “conservative substitutions” for one another.

TABLE 1
Amino Acid Residue Classes

| Amino Acid Class | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

An alternative set of conservative amino acid substitutions, delineated by six conservation groups, is provided in Table 2.

TABLE 2
Alternative Amino Acid Residue Substitution Groups

| | | |
|---|---|---|
| Alanine (A) | Serine (S) | Threonine (T) |
| Aspartic acid (D) | Glutamic acid (E) | |
| Asparagine (N) | Glutamine (Q) | |
| Arginine (R) | Lysine (K) | |
| Isoleucine (I) | Leucine (L) | Methionine (M) |
| Phenylalanine (F) | Tyrosine (Y) | Tryptophan (W) |

More conservative substitutions exist within the above-described amino acid residue classes, which also or alternatively can be suitable. Conservation groups for substitutions that are more conservative include: valine-leucine-isoleucine, phenylalanine-tyrosine, lysine-arginine, alanine-valine, and asparagine-glutamine. Thus, for example, the invention provides a polypeptide comprising an amino acid sequence that has at least 90, 91, 92, 93, 94, 95, 96, 97, 98, or 99% identity to SEQ ID NO:1 that differs from SEQ ID NO:1 by mostly (e.g., at least 50%), if not entirely by such more conservative amino acid substitutions.

Additional groups of amino acids substitutions that also can be suitable can be determined using the principles described in, e.g., Creighton (1984) PROTEINS: STRUCTURE AND MOLECULAR PROPERTIES (2d Ed. 1993), W.H. Freeman and Company. In some aspects, at least 33%, at least 50%, at least 65%, or more (e.g., at least 90, 95, 96, 97% or more) of the substitutions in a amino acid sequence variant comprise substitutions of one or more amino acid residues in a polypeptide sequence of the invention with residues that are within the same functional homology class (as determined by any suitable classification system, such as those described above) as the amino acid residues of the polypeptide sequence that they replace.

Conservatively substituted variations of a polypeptide sequence of the present invention include substitutions of a small percentage, typically less than 5%, more typically less than 4%, 3%, 2%, or 1%, of the amino acids of the sequence, with a conservatively selected amino acid of the same conservative substitution group.

One aspect of the invention pertains to a chimeric antigenic polypeptide comprising an antigenic polypeptide sequence having at least 90, 91, 92, 93, 94, 95, 96, 97, 98, 99%, or 100% amino acid sequence identity to a polypeptide sequence selected from the group of SEQ ID NOS:1-21 and 56-63, and wherein such polypeptide induces and/or promotes an immune response against an HIV-1 virus or pseudovirus. The immune response induced against the HIV-1 virus or pseudovirus can be any type of immune response, which can be manifested in any detectable manner. Immune responses include, e.g., a cellular immune response (e.g., a T cell immune response), a humoral (e.g., an antibody-associated and/or antibody-mediated) immune response, or both. The polypeptide may have the ability to induce and/or enhance an HIV-1-specific T cell proliferative response or production of at least one cytokine and/or may have an ability to bind anti-HIV-1 antibodies. Standard methods for evaluating such immune responses are known to those of skill in the art, and selected methods are described below.

Also provided are polypeptide variants of such a chimeric antigenic polypeptide, wherein the amino acid sequence of the polypeptide variant differs from the respective antigenic polypeptide sequence by one or more conservative amino acid residue substitutions, although non-conservative substitutions are sometimes permissible or even preferred (examples of such non-conservative substitutions are discussed further herein). For example, the sequence of the polypeptide variant can vary from such antigenic polypeptide sequence by one or more substitutions of amino acid residues in the antigenic polypeptide sequence with one or more amino acid residues having similar weight (i.e., a residue that has weight homology to the residue in the respective polypeptide sequence that it replaces). The weight (and correspondingly the size) of amino acid residues of a polypeptide can significantly impact the structure of the polypeptide. Weight-based conservation or homology is based on whether a non-identical corresponding amino acid is associated with a positive score on one of the weight-based matrices described herein (e.g., the BLOSUM50 matrix and PAM250 matrix). Similar to the above-described functional amino acid classes, naturally occurring amino acid residues can be divided into weight-based conservations groups (which are divided between “strong” and “weak” conservation groups). The eight commonly used weight-based strong conservation groups are Ser Thr Ala, Asn Glu Gln Lys, Asn His Gln Lys, Asn Asp Glu Gln, Gln His Arg Lys, Met Ile Leu Val, Met Ile Leu Phe, His Tyr, and Phe Tyr Trp. Weight-based weak conservation groups include Cys Ser Ala, Ala Thr Val, Ser Ala Gly, Ser Thr Asn Lys, Ser Thr Pro Ala, Ser Gly Asn Asp, Ser Asn Asp Glu Gln Lys, Asn Asp Glu Gln His Lys, Asn Glu Gln His Arg Lys, Phe Val Leu Ile Met, and His Phe Tyr. Some versions of the CLUSTAL W sequence analysis program provide an analysis of weight-based strong conservation and weak conservation groups in the output of an alignment, thereby offering a convenient technique for determining weight-based conservation (e.g., CLUSTAL W provided by the SDSC, which typically is used with the SDSC default settings). In some aspects, at least 33%, at least 50%, at least 65%, or more (e.g., at least 90%) of the substitutions in such polypeptide variant comprise substitutions wherein a residue within a weight-based conservation replaces an amino acid residue of the antigenic polypeptide sequence that is in the same weight-based conservation group. In other words, such a percentage of substitutions are conserved in terms of amino acid residue weight characteristics.

The sequence of a polypeptide variant can differ from the chimeric antigenic polypeptide sequence by one or more substitutions with one or more amino acid residues having a similar hydropathy profile (i.e., that exhibit similar hydrophilicity) to the substituted (original) residues of the antigenic polypeptide. A hydropathy profile can be determined using the Kyte & Doolittle index, the scores for each naturally occurring amino acid in the index being as follows: I (+4.5), V (+4.2), L (+3.8), F (+2.8), C (+2.5), M (+1.9); A (+1.8), G (−0.4), T (−0.7), S (−0.8), W (−0.9), Y (−1.3), P (−1.6), H (−3.2); E (−3.5), Q (−3.5), D (−3.5), N (−3.5), K (−3.9), and R (−4.5) (see, e.g., U.S. Pat. No. 4,554,101 and Kyte & Doolittle, J. Molec. Biol. 157:105-32 (1982) for further discussion). At least 75%, 85%, 90%, 91%, 92%, 93%, 94%, 95%, 96%, 97% 98%, or 99% of the amino acid residues in the immunogenic amino acid sequence that are not identical to the corresponding residues in the identical or functionally homologous immunogenic amino acid sequence disclosed herein (“most related homolog”), which homolog may be selected from any of SEQ ID NOS:1-21 and 56-63, exhibit less than a +/−2 change in hydrophilicity, including less than a +/−1 change in hydrophilicity and less than a +/−0.5 change in hydrophilicity with respect to the non-identical amino acid residue at the corresponding position in the most related homolog. The polypeptide may exhibit a total change in hydrophilicity with respect to its most related homolog selected from the group of SEQ ID NOS:1-21 and 56-63, of less than about 150, less than about 100, and/or less than about 50 (e.g., less than about 30, less than about 20, or less than about 10).

The polypeptide may comprise a sequence having at least 90%, 91%, 92%, 93%, 94%, 95%, 96% 97%, 98%, 99%, 99.5%, or 100% identity to at least one sequence selected from the group consisting of SEQ ID NOS:1-21 and 56-63, wherein at least 90%, 91%, 92%, 95%, 96%, 97%, 98%, 99% or 100% of the amino acid residues in the composition have a Kyte & Doolittle hydropathy score of above 0, and more preferably of at least about 1.

Examples of typical amino acid substitutions that retain similar or identical hydrophilicity include arginine-lysine substitutions, glutamate-aspartate substitutions, serine-threonine substitutions, glutamine-asparagine substitutions, and valine-leucine-isoleucine substitutions. Algorithms and software, such as the GREASE program available through the SDSC, provide a convenient way for quickly assessing the hydropathy profile of an amino acid sequence. Because a substantial proportion (e.g., at least about 33%), if not most (at least 50%) or nearly all (e.g., about 65, 80, 90, 95, 96, 97, 98, 99%) of the amino acid substitutions in the sequence of a polypeptide variant often will have a similar hydropathy score as the amino acid residue that they replace in the antigenic (reference) polypeptide sequence, the sequence of the polypeptide variant is expected to exhibit a similar GREASE program output as the antigenic polypeptide sequence. For example, in a particular aspect, a polypeptide variant of SEQ ID NO:1 may be expected to have a GREASE program (or similar program) output that is more like the GREASE output obtained by inputting the polypeptide sequence of SEQ ID NO:1 than that obtained by using a WT gp120 polypeptide (e.g., JRCSF gp120 polypeptide), which can be determined by visual inspection or computer-aided comparison of the graphical (e.g., graphical overlay/alignment) and/or numerical output provided by subjecting the test variant sequence and SEQ ID NO:1 to the program.

The conservation of amino acid residues in terms of functional homology, weight homology, and hydropathy characteristics, also apply to other polypeptide sequence variants provided by the invention, including, but not limited to, e.g., polypeptide sequence variants of a polypeptide sequence selected from the group consisting of SEQ ID NOS:1-21 and 56-63, which are discussed further herein.

In a particular aspect, the invention includes at least one such polypeptide variant comprising an amino acid sequence that differs from an antigenic polypeptide sequence selected from the group of SEQ ID NOS:1-21 and 56-63, wherein the amino acid sequence of the variant has at least one such amino acid residue substitution selected according to weight-based conservation or homology or similar hydropathy profile as discussed above. Such polypeptide variants described above typically induce at least one type of immune response against HIV-1 as described previously and in greater detail below in the Examples.

Signal Peptide Sequences

Polypeptides of the invention can also further comprise any suitable number and type of additional amino acid sequences, such as one or more peptide fragments. In one embodiment, such a polypeptide of the invention further comprises a signal peptide. Generally, the signal peptide directs the recombinant polypeptide to the endoplasmic reticulum when the recombinant polypeptide is expressed in an animal cell. A signal sequence that directs organelle trafficking and/or secretion of at least a portion of the polypeptide upon expression in a cell may be included. Such sequences are typically present in the immature (i.e., not fully processed) form of the polypeptide, and are subsequently removed/degraded by cellular proteases to arrive at the mature form of the protein. For example, a gp120 polypeptide variant of the invention can include any suitable signal sequence or combinations of signal sequences that direct the polypeptide to intracellular compartments, such as a sequence that directs the polypeptide to be transported (e.g., translocated) into (e.g., such that the protein is processed by and released from) the endoplasmic reticulum or secretory pathway (e.g., the ER, golgi, and other secretory related organelles and cellular compartments), the nucleus, and/or which directs the polypeptide to be secreted from the cell, translocated in a cellular membrane, or target a second cell apart from the cell the protein is secreted from. In this respect, the polypeptide can include an intracellular targeting sequence (or “sorting signal”) that directs the polypeptide to an endosomal and/or lysosomal compartment(s) or other compartment rich in MHC II to promote CD4+ and/or CD8+ T cell presentation and response, such as a lysosomal/endosomal-targeting sorting signal derived from lysosomal associated membrane protein 1 (e.g., LAMP-1—see, e.g., Wu et al. Proc. Natl. Acad. Sci. USA 92:1161-75 (1995) and Ravipraskash et al., Virology 290:74-82 (2001)), a portion or homolog thereof (see, e.g., U.S. Pat. No. 5,633,234), or other suitable lysosomal, endosomal, and/or ER targeting sequence (see, e.g., U.S. Pat. No. 6,248,565). In some aspects, it may desirable for the intracellular targeting sequence to be located near or adjacent to a proven/identified anti-HIV virus T-cell epitope sequence(s) within the polypeptide, which can be identified by techniques known in the art, thereby increasing the likelihood of T cell presentation of polypeptide fragments that comprise such epitope(s). Such polypeptides may be expressed from an isolated, recombinant, or synthetic DNA or RNA delivered to a host cell by one or more of the nucleotide or viral nucleotide transfer vectors, including, e.g., one or more of the gene transfer vectors, described further herein.

The polypeptide may comprise a signal sequence that directs the polypeptide to the endoplasmic reticulum (ER) (e.g., facilitates ER translocation of the polypeptide) when the polypeptide is expressed in a mammalian cell. The polypeptide can comprise any suitable ER-targeting sequence. Many ER-targeting sequences are known in the art. Examples of such signal sequences are described in U.S. Pat. No. 5,846,540. Commonly employed ER/secretion signal sequences include the STII or Ipp signal sequences of E. coli, yeast alpha factor signal sequence, and mammalian viral signal sequences such as herpes virus gD signal sequence. Further examples of signal sequences are described in, e.g., U.S. Pat. Nos. 4,690,898, 5,284,768, 5,580,758, 5,652,139, and 5,932,445. Suitable signal sequences can be identified using skill known in the art. For example, the SignalP program (described in, e.g., Nielsen et al. (1997) Protein Engineering 10:1-6), which is publicly available through the Center for Biological Sequence Analysis at the website designated cbs.dtu.dk/services/SignalP, or similar sequence analysis software capable of identifying signal-sequence-like domains can be used. Related techniques for identifying suitable signal peptides are provided in Nielsen et al., Protein Eng. 10(1):1-6 (1997). Sequences can be manually analyzed for features commonly associated with signal sequences, as described in, e.g., European Patent Application (Appn) No. 0 621 337, Zheng and Nicchitta (1999) J. Biol. Chem. 274(51): 36623-30, and Ng et al. (1996) J. Cell Biol. 134(2):269-78.

Additional Aspects

Any polypeptide of the invention may be present as part of a larger polypeptide sequence, e.g. a fusion protein, such as occurs upon the addition of one or more domains or subsequences for stabilization or detection or purification of the polypeptide. A polypeptide purification subsequence may include, e.g., an epitope tag, a FLAG tag, a polyhistidine sequence, a GST fusion, or any other detection/purification subsequence or “tag” known in the art. These additional domains or subsequences either have little or no effect on the activity of the polypeptide of the invention, or can be removed by post synthesis processing steps such as by treatment with a protease, inclusion of an intein, or the like.

Any polypeptide of the invention may also comprise one or more modified amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, and/or an amino acid conjugated to an organic derivatizing agent. The presence of modified amino acids may be advantageous in, for example, (a) increasing polypeptide serum half-life and/or functional in vivo half-life, (b) reducing polypeptide antigenicity, (c) increasing polypeptide storage stability, or (d) increasing bioavailability, e.g. increasing the AUC_{sc}. Amino acid(s) are modified, for example, co-translationally or post-translationally during recombinant production (e.g., N-linked glycosylation at N-X-S/T motifs during expression in mammalian cells) or modified by synthetic means.

Polypeptides of the invention described herein can be further modified in a variety of ways by, e.g., post translational modification and/or synthetic modification or variation. For example, polypeptides of the invention may be suitably glycosylated, typically via expression in a mammalian cell. In one aspect, the invention provides glycosylated polypeptides which induce an immune response against an HIV virus or pseudovirus based on an HIV envelope protein as described herein, wherein said glycosylated polypeptides comprise the polypeptide sequence of any of SEQ ID NOS:1-21, 56-63, 107-110 and 131-134.

The invention also provides deglycosylated polypeptide variants which induce an immune response against an HIV virus or pseudovirus based on an HIV envelope protein as described in Example 11 herein, wherein said deglycosylated polypeptide variants comprise the polypeptide sequence of any of SEQ ID NOS:1-21, 56-63, 107-110 and 131-134, and an amino acid substitution in a glycosylation motif (N-X-S/T) which eliminates N-linked glycosylation at one or more glycosylation sites selected from N156, N188, N197, N276, N295, N301, N332, N386, N448, and N461, wherein the amino acid residues are numbered according to the amino acid residues of the recombinant gp120-HXB2 envelope protein (SEQ ID NO:54) as shown in FIGS. 10A-10F, and wherein the deglycosylated polypeptide variant induces the production of neutralizing antibodies against at least one HIV-1 virus in a subject to whom an effective amount of the variant is administered.

In one embodiment, the amino acid substitution is a substitution of the N (Asn) in the glycosylation motif with a different amino acid, such as a Q (Gln). In another embodiment, the amino acid substitution is a substitution of the Ser (S) or Thr (T) in the glycosylation motif with a different amino acid, such as an Ala (A). The deglycosylated polypeptide variant may comprise substitutions which eliminate N-linked glycosylation at 2, 3, 4, 5, 6, 7, 8, 9 or a1110 of said glycosylation sites. In one particular aspect, the deglycosylated polypeptide variant comprises the substitution N461Q. As described in Example 11, some such deglycosylated variants induce an increased immune response against at least one human immunodeficiency virus type 1 (HIV-1 virus) or pseudovirus, compared to the immune response induced by the parent polypeptide (i.e., the polypeptide lacking substitutions at the glycosylation sites).

The polypeptides of the invention can be subject to any number of additional forms suitable of post translational and/or synthetic modification or variation. For example, the invention provides protein mimetics of the polypeptides of the invention. Peptide mimetics are described in, e.g., U.S. Pat. No. 5,668,110 and the references cited therein.

In another aspect, a polypeptide of the invention can be modified by the addition of protecting groups to the side chains of one or more the amino acids of the fusion protein. Such protecting groups can facilitate transport of the fusion peptide through membranes, if desired, or through certain tissues, for example, by reducing the hydrophilicity and increasing the lipophilicity of the peptide. Examples of suitable protecting groups include ester protecting groups, amine protecting groups, acyl protecting groups, and carboxylic acid protecting groups, which are known in the art (see, e.g., U.S. Pat. No. 6,121,236). Synthetic fusion proteins of the invention can take any suitable form. For example, the fusion protein can be structurally modified from its naturally occurring configuration to form a cyclic peptide or other structurally modified peptide.

Polypeptides of the invention also can be linked to one or more nonproteinaceous polymers, typically a hydrophilic synthetic polymer, e.g., polyethylene glycol (PEG), polypropylene glycol, or polyoxyalkylene, using techniques well known in the art, such as described in, e.g., U.S. Pat. Nos. 4,179,337, 4,301,144, 4,496,689, 4,640,835, 4,670,417, and 4,791,192, or a similar polymer such as polyvinylalcohol or polyvinylpyrrolidone (PVP).

As discussed above, polypeptides of the invention can commonly be subject to glycosylation. Polypeptides of the invention can further be subject to (or modified such that they are subjected to) other forms of post-translational modification including, e.g., hydroxylation, lipid or lipid derivative-attachment, methylation, myristylation, phosphorylation, and sulfation. Other post-translational modifications that a polypeptide of the invention can be rendered subject to include acetylation, acylation, ADP-ribosylation, amidation, covalent attachment of flavin, covalent attachment of a heme moiety, covalent attachment of a nucleotide or nucleotide derivative, covalent attachment of phosphotidylinositol, cross-linking, cyclization, disulfide bond formation, demethylation, formylation, GPI anchor formation, iodination, oxidation, proteolytic processing, prenylation, racemization, selenoylation, arginylation, and ubiquitination. Other common protein modifications are described in, e.g., Creighton, supra, Seifter et al., Meth. Enzymol. 18:626-646 (1990), and Rattan et al., Ann. NY Acad. Sci. 663:48-62 (1992). Post-translational modifications for polypeptides expressed from nucleic acids in host cells vary depending what kind of host or host cell type the peptide is expressed in. For instance, glycosylation often does not occur in bacterial hosts such as E. coli and varies considerably in baculovirus systems as compared to mammalian cell systems. Accordingly, when glycosylation is desired (which usually is the case for most polypeptides of the present invention), a polypeptide should be expressed (produced) in a glycosylating host, generally a eukaryotic cell (e.g., a mammalian cell or an insect cell). Modifications to the polypeptide in terms of post-translational modification can be verified by any suitable technique, including, e.g., x-ray diffraction, NMR imaging, mass spectrometry, and/or chromatography (e.g., reverse phase chromatography, affinity chromatography, or GLC).

The polypeptide also or alternatively can comprise any suitable number of non-naturally occurring amino acids (e.g., β amino acids) and/or alternative amino acids (e.g., selenocysteine), or amino acid analogs, such as those listed in the MANUAL OF PATENT EXAMINING PROCEDURE §2422 (7^{th} Revision—2000), which can be incorporated by protein synthesis, such as through solid phase protein synthesis (as described in, e.g., Merrifield, Adv. Enzymol. 32:221-296 (1969) and other references cited herein). A polypeptide of the invention can further be modified by the inclusion of at least one modified amino acid. The inclusion of one or more modified amino acids may be advantageous in, for example, (a) increasing polypeptide serum half-life, (b) reducing polypeptide antigenicity, or (c) increasing polypeptide storage stability. Amino acid(s) are modified, for example, co-translationally or post-translationally during recombinant production (e.g., N-linked glycosylation at N-X-S/T motifs during expression in mammalian cells) or modified by synthetic means. Non-limiting examples of a modified amino acid include a glycosylated amino acid, a sulfated amino acid, a prenylated (e.g., farnesylated, geranylgeranylated) amino acid, an acetylated amino acid, an acylated amino acid, a PEGylated amino acid, a biotinylated amino acid, a carboxylated amino acid, a phosphorylated amino acid, and the like. References adequate to guide one of skill in the modification of amino acids are replete throughout the literature. Example protocols are found in Walker (1998) Protein Protocols on CD-ROM Humana Press, Towata, N.J. The modified amino acid may be selected from a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent.

Recently, the production of fusion proteins comprising a prion-determining domain has been used to produce a protein vector capable of non-Mendelian transmission to progeny cells (see, e.g., Li et al., J. Mol. Biol. 301(3):567-73 (2000)). The inclusion of such prion-determining sequences in a fusion protein comprising immunogenic polypeptide sequences of the invention is contemplated, ideally to provide a hereditable protein vector comprising the fusion protein that does not require a change in the host's genome.

The invention further provides polypeptides having the above-described characteristics that further comprise additional amino acid sequences that impact the biological function (e.g., immunogenicity, targeting, and/or half-life) of the polypeptide. For example, in one aspect the invention provides a polypeptide comprising an immunogenic polypeptide sequence of the invention (including, e.g., but not limited to, any of SEQ ID NOS:1-21 and 56-63 or variant thereof as described herein) and the polypeptide sequence of an Interleukin, such as Interleukin-2 (IL-2), or a fragment thereof that enhances the ability of the polypeptide to generate an immune response to an HIV.

A polypeptide of the invention may further include a targeting sequence other than, or in addition to, a signal sequence. For example, the polypeptide can comprise a sequence that targets a receptor on a particular cell type (e.g., a monocyte, dendritic cell, or associated cell) to provide targeted delivery of the polypeptide to such cells and/or related tissues. Signal sequences are described above, and include membrane localization/anchor sequences (e.g., stop transfer sequences, GPI anchor sequences), and the like.

Another possible advantageous fusion partner for a polypeptide of the invention is an immunogenic heat shock protein (HSP) or portion thereof, such as HSP65, HSP70, HSP110, and gp96 (see, e.g., U.S. Pat. No. 6,335,183).

Also provided is a fusion protein comprising a polypeptide of the invention and a receptor amino acid sequence, such that the polypeptide acts as a chimeric immune receptor (CIR—see, e.g., Patel et al.—Cancer Gene Ther. (2000) 7(8):1127-34 for discussion of similar CIR molecules).

A particularly useful fusion partner for a polypeptide of the invention is a peptide sequence that facilitates purification of the polypeptide, e.g., a polypeptide purification subsequence. A polynucleotide of the invention may comprise a coding sequence fused in-frame to a marker amino acid sequence that, e.g., facilitates purification of the encoded polypeptide. Such purification facilitating peptide domains or polypeptide purification subsequences include, but are not limited to, metal chelating peptides, such as histidine-tryptophan modules that allow purification on immobilized metals, such as a hexa-histidine peptide or other a polyhistidine sequence, a sequence encoding such a tag is incorporated in the pQE vector available from QIAGEN, Inc. (Chatsworth, Calif.), a sequence which binds glutathione (e.g., glutathione-S-transferase (GST)), a hemagglutinin (HA) tag (corresponding to an epitope derived from the influenza hemagglutinin protein; Wilson et al., Cell 37:767 (1984)), maltose binding protein sequences, the FLAG epitope utilized in the FLAGS extension/affinity purification system (Immunex Corp, Seattle, Wash.)(commercially available FLAG epitopes also are available through Kodak (New Haven, Conn.)), an E-epitope tag (E-tag), thioredoxin (TRX), avidin, and the like. Purification-facilitating epitope tags have been described in the art (see, e.g., Whitehorn et al., Biotechnology 13:1215-19 (1995)). A polypeptide can include an e-his tag, which may comprise a polyhistidine sequence and an anti-e-epitope sequence (Pharmacia Biotech Catalog); e-his tags can be made by standard techniques. The inclusion of a protease-cleavable polypeptide linker sequence between the purification domain and the polypeptide is useful to facilitate purification. The histidine residues facilitate purification on IMIAC (immobilized metal ion affinity chromatography (IMAC), as described in Porath et al. Protein Expression and Purification 3:263-281 (1992)), while the enterokinase cleavage site provides a method for separating the polypeptide from the fusion protein. pGEX vectors (Promega; Madison, Wis.) can also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusion) followed by elution in the presence of free ligand. Additional examples of polypeptide purification facilitating subsequences and the use thereof for protein purification are described in, e.g., International Patent Application Publication No. WO 00/15823. After expression of the polypeptide of interest and isolation thereof by such fusion partners or otherwise as described above, protein refolding steps can be used, as desired, in completing configuration of the mature polypeptide.

A fusion protein of the invention also can include one or more additional peptide fragments or peptide portions which promote detection of the fusion protein. For example, a reporter peptide fragment or portion (e.g., green fluorescent protein (GFP), β-galactosidase, or a detectable domain thereof) can be incorporated in the fusion protein. Additional marker molecules that can be conjugated to the polypeptide of the invention include radionuclides, enzymes, fluorophores, small molecule ligands, and the like. Such detection-promoting fusion partners are particularly useful in fusion proteins used in diagnostic techniques discussed elsewhere herein.

In another aspect, a polypeptide of the invention can comprise a fusion partner that promotes stability of the polypeptide, secretion of the polypeptide (other than by signal targeting), or both. For example, the polypeptide can comprise an immunoglobulin (Ig) domain, such as an IgG polypeptide comprising an Fc hinge, a CH2 domain, and a CH3 domain, that promotes stability and/or secretion of the polypeptide.

The fusion protein peptide fragments or peptide portions can be associated in any suitable manner. The various polypeptide fragments or portions of the fusion protein may be covalently associated (e.g., by means of a peptide or disulfide bond). The polypeptide fragments or portions can be directly fused (e.g., the C-terminus of an antigenic or immunogenic sequence of the invention can be fused to the N-terminus of a purification sequence or heterologous immunogenic sequence). The fusion protein can include any suitable number of modified bonds, e.g., isosteres, within or between the peptide portions. Alternatively or additionally, the fusion protein can include a peptide linker between one or more polypeptide fragments or portions that includes one or more amino acid sequences not forming part of the biologically active peptide portions. Any suitable peptide linker can be used. Such a linker can be any suitable size. Typically, the linker is less than about 30 amino acid residues, less than about 20 amino acid residues, and/or less than 10 amino acid residues. The linker predominantly may comprise or consist of neutral amino acid residues. Suitable linkers are generally described in, e.g., U.S. Pat. Nos. 5,990,275, 6,010,883, 6,197,946, and European Patent Application 0 035 384. If separation of peptide fragments or peptide portions is desirable a linker that facilitates separation can be used. An example of such a linker is described in U.S. Pat. No. 4,719,326. “Flexible” linkers, which are typically composed of combinations of glycine and/or serine residues, can be advantageous. Examples of such linkers are described in, e.g., McCafferty et al., Nature 348:552-554 (1990), Huston et al., Proc. Natl. Acad. Sci. USA 85:5879-5883 (1988), Glockshuber et al., Biochemistry 29:1362-1367 (1990), and Cheadle et al., Molecular Immunol. 29:21-30 (1992), Bird et al., Science 242:423-26 (1988), and U.S. Pat. Nos. 5,672,683, 6,165,476, and 6,132,992.

The use of a linker also can reduce undesired immune response to the fusion protein created by the fusion of the two peptide fragments or peptide portions, which can result in an unintended MHC I and/or MHC II epitope being present in the fusion protein. In addition to the use of a linker, identified undesirable epitope sequences or adjacent sequences can be PEGylated (e.g., by insertion of lysine residues to promote PEG attachment) to shield identified epitopes from exposure. Other techniques for reducing immunogenicity of the fusion protein of the invention can be used in association with the administration of the fusion protein include the techniques provided in U.S. Pat. No. 6,093,699.

Making Polypeptides

Recombinant methods for producing and isolating polypeptides of the invention are described below. In addition to recombinant production, the polypeptides may be produced by direct peptide synthesis using solid-phase techniques (see, e.g., Stewart et al. (1969) Solid-Phase Peptide Synthesis, W.H. Freeman Co, San Francisco; Merrifield (1963) J. Am. Chem. Soc 85:2149-2154). Peptide synthesis may be performed using manual techniques or by automation. Automated synthesis may be achieved, for example, using Applied Biosystems 431A Peptide Synthesizer (Applied Biosystems, Foster City, Calif.) in accordance with the instructions provided by the manufacturer. For example, subsequences may be chemically synthesized separately and combined using chemical methods to provide polypeptides of the invention (e.g., gp120 full-length polypeptide variants or gp120 core polypeptide variants or core+V1V2 polypeptide variants or core+V3 polypeptide variants or gp120ΔV3 polypeptide variants or gp120ΔV1V2V3 polypeptide variants of the invention or fragments thereof). Alternatively, such sequences may be ordered from any number of companies that specialize in production of polypeptides. Most commonly, polypeptides of the invention are produced by expressing coding nucleic acids and recovering polypeptides, e.g., as described below.

The invention provides methods for producing polypeptides of the invention. One such method comprises introducing into a population of cells any nucleic acid described herein, which is operatively linked to a regulatory sequence effective to produce the encoded polypeptide, culturing the cells in a culture medium to produce the polypeptide, and isolating the polypeptide from the cells or from the culture medium. An amount of nucleic acid sufficient to facilitate uptake by the cells (transfection) and/or expression of the polypeptide is utilized. The culture medium can be any described herein and in the Examples. Additional media are known to those of skill in the art. The nucleic acid is introduced into such cells by any delivery method described herein, including, e.g., injection, gene gun, electroporation (e.g., DNA electroporation device, Inovio Biomedical Corp. (San Diego)), passive uptake, etc. The nucleic acid of the invention may be part of a vector, such as a recombinant expression vector, including a DNA plasmid vector, viral vector, or any vector described herein. The nucleic acid or vector comprising a nucleic acid of the invention may be prepared and formulated as described herein, above, and in the Examples below. Such a nucleic acid or expression vector may be introduced into a population of cells of a mammal in vivo, or selected cells of the mammal (e.g., tumor cells) may be removed from the mammal and the nucleic acid expression vector introduced ex vivo into the population of such cells in an amount sufficient such that uptake and expression of the encoded polypeptide results. Or, a nucleic acid or vector comprising a nucleic acid of the invention is produced using cultured cells in vitro. In one aspect, the method of producing a polypeptide of the invention comprises introducing into a population of cells a recombinant expression vector comprising any nucleic acid described herein in an amount and formula such that uptake of the vector and expression of the polypeptide will result; administering the expression vector into a mammal by any introduction/delivery format described herein; and isolating the polypeptide from the mammal or from a byproduct of the mammal. Suitable host cells, expression vectors, methods for transfecting host cells with an expression vector comprising a nucleic acid sequence encoding a polypeptide of the invention, cell cultures, and procedures for producing and recovering such polypeptide from a cell culture are described in detail below in the section entitled “Nucleic Acids of the Invention.” Additional methods of production are discussed infra.

As noted above, polypeptides of the invention can be subject to various changes, such as one or more amino acid or nucleic acid insertions, deletions, and substitutions, either conservative or non-conservative, including where, e.g., such changes might provide for certain advantages in their use, e.g., in their therapeutic or prophylactic use or administration or diagnostic application. Procedures for making variants of polypeptides by using amino acid substitutions, deletions, insertions, and additions are routine in the art. Polypeptides and variants thereof having the desired ability to induce an immune response against an HIV virus or pseudovirus (e.g., ability to induce HIV-specific antibodies, anti-HIV antibody binding properties, and/or HIV-specific T cell response) are readily identified by assays known to those of skill in the art and by the assays described herein.

The nucleic acids of the invention, discussed in greater detail infra, can also be subject to various changes, such as one or more substitutions of one or more nucleic acids in one or more codons such that a particular codon encodes the same or a different amino acid, resulting in either a conservative or non-conservative substitution, or one or more deletions of one or more nucleic acids in the sequence. The nucleic acids can also be modified to include one or more codons that provide for optimum expression in an expression system (e.g., mammalian cell or mammalian expression system), while, if desired, said one or more codons still encode the same amino acid(s). Procedures for making variants of nucleic acids by using nucleic acid substitutions, deletions, insertions, and additions, and degenerate codons, are routine in the art, and nucleic acid variants encoding polypeptides having the desired properties described herein (e.g., an ability to induce an immune response against an HIV virus) are readily identified using the assays described herein. Such nucleic acid changes might provide for certain advantages in their therapeutic or prophylactic use or administration, or diagnostic application. In one aspect, the nucleic acids and polypeptides can be modified in a number of ways so long as they comprise a sequence substantially identical to the sequence of a respective gp120 polypeptide variant-encoding nucleic acid or gp120 polypeptide variant of the invention.

Nucleic Acids of the Invention

The invention also provides isolated, recombinant, or non-naturally occurring nucleic acids that are useful in a number of contexts including, e.g., the expression of at least one polypeptide that induces an immune response against an HIV virus, such as HIV-1, or an HIV pseudovirus.

In one aspect, the invention provides an isolated or recombinant nucleic acid comprising a nucleotide sequence encoding any at least one of the polypeptides of the invention described above and elsewhere herein. The invention also provides an isolated or recombinant nucleic acid comprising a nucleotide sequence encoding a combination of two or more of any of the polypeptides of the invention described above and elsewhere herein. Also included is a nucleic acid that encodes any polypeptide of the invention, such as, e.g., a chimeric HIV-1 gp120 polypeptide variant, which comprises a sequence of codons substantially optimized for expression in a mammalian host, such as a human.

The invention includes an isolated or recombinant nucleic acid comprising a polynucleotide sequence that has at least 75, 80, 85, 90, 91, 92, 93, 94, 95, 96, 97, 98, 99, 99.5 or 100% nucleic acid sequence identity or sequence similarity to a polynucleotide sequence that encodes a polypeptide comprising a an amino acid sequence having at least 85, 90, 91, 92, 93, 94, 95, 96, 97, 98, 99, 99.5, or 100% to a polypeptide sequence selected from the group consisting of SEQ ID NOS:1-21 and 56-63, or a complementary polynucleotide sequence thereof. Such nucleic acids may encode a polypeptide that induces an immune response in a subject to whom an effective amount of such nucleic acid(s) is administered (or in which such polypeptide is expressed) against an HIV virus, including a human HIV virus, such as, e.g., HIV-1, or an HIV-1 pseudovirus. Such nucleic acids may express polypeptides that induce an immune response against an HIV virus. Some such nucleic acids may encode a polypeptide that induces an immune response against two or more HIV viruses (or two or more HIV pseudoviruses), including, but not limited to, two or more HIV viruses or pseudoviruses (e.g., HIV-1 viruses or pseudoviruses) of the same subtype or of a different subtype or any combination thereof. The immune response may comprise a humoral immune response (e.g., antibody response) or cellular immune response (e.g., T cell response) or both. The induced immune response may comprise a neutralizing antibody response. Some such nucleic acids express a polypeptide that induces an anti-HIV-1 neutralizing antibody response and/or an HIV-1 specific T cell response. The immune response induced by the effective amount of the administered nucleic acid(s) may be effective to prevent or inhibit HIV infection. Some nucleic acids of the invention express a polypeptide that induces in the subject to an effective amount of whom such nucleic acid(s) has been administered the production of antibodies capable of binding to at least one HIV-1 virus or pseudovirus.

Determining the level of identity of a portion of a nucleic acid to its target (i.e., SEQ ID NO:23) can be accomplished by using any of the sequence alignment techniques and percent identity determination techniques described elsewhere herein (by, e.g., but not limited to, using LFASTA, LALIGN, and/or aligning sequences manually in an optimal local sequence alignment).

In another aspect, the invention provides an isolated or recombinant nucleic acid comprising a polynucleotide sequence that has at least 80, 85, 90, 91, 92, 93, 94, 95, 96, 97, 98, 99, 99.5, or 100% nucleic acid sequence identity to a polynucleotide sequence selected from the group consisting of SEQ ID NOS:23-50 or 64-79, or a complementary polynucleotide sequence thereof, wherein the encoded polypeptide induces an immune response against at least one HIV (e.g., HIV-1) virus or pseudovirus in a subject to whom an amount of the nucleic acid effective to induce such response is administered. The induced immune response may comprise a humoral and/or T cell immune response (such as, e.g., a B cell and T cell immune response to HIV virus in a human host). The induced immune response may be a neutralizing antibody response and/or may be against one or more HIV-1 viruses or pseudoviruses of the same or different subtypes. In one aspect, such nucleic acid encodes a polypeptide that induces a neutralizing antibody response against multiple HIV-1 viruses or HIV-1 pseudoviruses of the same subtype (e.g., subtype B) or one or more different subtypes (e.g., B, C, D, etc.) and/or T cell proliferation response specific to multiple HIV-1 viruses or HIV-1 pseudoviruses of the same subtype or one or more different subtypes or clade (such as 2, 3, 4, 5, 6, or 7 subtypes or clades). In one embodiment, the nucleic acid consists of or consists essentially of the nucleotide sequence of SEQ ID NO:23, 24, or 27, or a complementary nucleotide sequence thereof.

In another aspect, the invention provides an isolated or recombinant nucleic acid comprising a nucleotide sequence that encodes a polypeptide having at least 80, 85, 90, 91, 92, 93, 94, 95, 96, 97, 98, 99, 99.5, or 100% sequence identity to a polypeptide sequence selected from the group consisting of SEQ ID NOS:1-21 and 56-63, or a complementary nucleotide sequence thereof, wherein the polypeptide induces in a subject to whom an effective amount of the nucleic acid is administered an immune response against at one or more HIV viruses (e.g., HIV-1) of the same or different subtypes or of any combination of subtypes. The immune response may comprise an antibody response or T cell response. The immune response may comprise a neutralizing antibody response.

The invention also includes an isolated or recombinant nucleic acid comprising a polypeptide sequence having at least 90, 91, 92, 93, 94, 95, 96, 97, 98, 99, 99.5, or 100% nucleic acid sequence identity to a nucleotide sequence that encodes a polypeptide comprising a polypeptide sequence selected from the group consisting of SEQ ID NOS:1-21 and 56-63, or a complementary nucleotide sequence thereof, wherein the polypeptide induces in a subject to whom an effective amount of the nucleic acid is administered an immune response against at least one HIV virus, including a human HIV virus (e.g., HIV-1), or HIV pseudovirus (e.g., HIV-1 pseudovirus) of the same or different subtypes or any combination of subtypes. Some such nucleic acids encode a polypeptide that induces an immune response against two or more HIV viruses, including, but not limited to, two or more HIV viruses of the same subtype or of a different subtype or any combination thereof. The immune response may comprise a humoral or cellular response (including, e.g., an anti-HIV neutralizing antibody response or HIV-specific T cell immune response) or both, and may prevent or inhibit HIV infection. The immune response may comprise a neutralizing antibody response. Some such nucleic acids express a polypeptide that induces in the subject production of antibodies capable of binding to at least one HIV-1 virus or HIV-1 pseudovirus (e.g., capable of binding up to 2, 3, 4, 5, 6, 7, 8, 9 or 10 different HIV-1 viruses or pseudoviruses).

In another aspect, the invention provides an isolated or recombinant nucleic acid comprising a nucleotide sequence that encodes a fragment of a gp120 variant polypeptide sequence, the gp120 variant polypeptide sequence having at least 90, 91, 92, 93, 94, 95, 96, 97, 98, 99, 99.5 or 100% sequence identity to a sequence selected from the group consisting of SEQ ID NOS:1-7 and 56-63, wherein said fragment induces in a subject to whom an effective amount of said fragment is administered the production of neutralizing antibodies against at least one HIV-1 virus (of the same or different subtypes), and said fragment comprises at least those amino acid residues of the gp120 variant polypeptide sequence located at positions corresponding by reference to amino acid residues of regions C2, C3, V4, C4, and V5 of the HIV-1 gp120-HXB2 envelope protein sequence (SEQ ID NO:54) shown in FIGS. 10A-10F, wherein the amino acid residues of the fragment are numbered by reference to amino acid residues of the gp120-HXB2 envelope protein, or a complementary nucleotide sequence thereof.

Also provided is a isolated or recombinant nucleic acid that encodes a polypeptide comprising a polypeptide sequence having at least 90, 91, 92, 93, 94, 95, 96, 97, 98, 99, or 100% sequence identity to a sequence selected from the group consisting of SEQ ID NOS:1-21 and 56-63, wherein the polypeptide binds to at least one HIV-1 neutralizing antibody and/or non-neutralizing antibody, or a complementary nucleotide sequence thereof. The polypeptide may have a binding affinity for the HIV-1 neutralizing antibody that is about equal to or greater than the binding affinity of a corresponding HIV-1 gp120 full-length or core envelope polypeptide (e.g., JRCSF gp120 full-length or core protein) for the HIV-1 neutralizing antibody (e.g., mAb IgG1b12; 2G12). The polypeptide may have a binding affinity for an HIV-1 non-neutralizing antibody that is lower than the binding affinity of a corresponding HIV-1 gp120 full-length or core Env polypeptide for the HIV-1 non-neutralizing antibody (e.g., mAb b3 or b6). In some instances, the polypeptide exhibits a b12/b3 binding affinity ratio that is greater than the b12/b3 binding affinity ratio of an HIV-1 gp120 polypeptide, a b12/b6 binding affinity ratio that is greater than the b12/b6 binding affinity ratio of a corresponding HIV-1 gp120 full-length or core Env polypeptide.

In another aspect, the invention provides an isolated or recombinant nucleic acid that induces in a subject to whom such an effective amount of nucleic acid is administered an immune response against at least one HIV virus or HIV pseudovirus, wherein the nucleic acid comprises a polynucleotide sequence having at least 85, 90, 91, 92, 93, 94, 95, 96, 97, 98, 99, or 100% sequence identity to at least one sequence selected from the group consisting of SEQ ID NOS:23-50 and 64-79, or a complementary polynucleotide sequence thereof. For some such nucleic acids, the immune response comprises an anti-HIV-1 neutralizing antibody response or an HIV-specific T cell response or both. The immune response may be against at least two HIV-1 viruses or pseudoviruses of the same or different subtypes.

In another aspect, the invention provides an isolated or recombinant nucleic acid that induces in a subject to whom an effective amount of the nucleic acid is administered an immune response against at least one HIV virus or HIV pseudovirus, wherein the nucleic acid comprises a polynucleotide sequence which encodes a polypeptide comprising a first, a second, a third, a fourth and a fifth subsequence of a gp120 variant sequence, the gp120 variant sequence comprising an amino acid sequence having at least 85, 90, 91, 92, 93, 94, 95, 96, 97, 98, 99, or 100% sequence identity to a polypeptide sequence selected from the group consisting of SEQ ID NOS:1-21 and 56-63, wherein: (a) the first subsequence of the gp120 variant sequence comprises a sequence corresponding by reference to amino acid residues 83-127 of the C1 region of the recombinant HXB2 gp120 envelope protein (SEQ ID NO:54) shown in FIGS. 10A-10F, wherein the C-terminus of the first subsequence is covalently linked by a peptide bond to the N-terminus of a first linker peptide; (b) the second subsequence of the gp120 variant sequence corresponds by reference to the C2 region of the recombinant HXB2 gp120 envelope protein (SEQ ID NO:54) shown in FIGS. 10A-10F, wherein the N-terminus of the second subsequence is covalently linked by a peptide bond to the C-terminus of the first linker peptide, and the C-terminus of the second subsequence is covalently linked by a peptide bond to the N-terminus of a second linker peptide or a gp120 V3 region sequence; (c) the third subsequence of the gp120 variant sequence corresponds by reference to the C3 region of the recombinant HXB2 gp120 envelope protein (SEQ ID NO:54) shown in FIGS. 10A-10F, and the N-terminus of the third subsequence is covalently linked by a peptide bond to the C-terminus of the second linker polypeptide or the gp120 V3 region sequence, and the C-terminus of the third subsequence is covalently linked by a peptide bond to the N-terminus of a gp120 V4 region sequence; (d) the fourth subsequence of the gp120 variant sequence corresponds by reference to the C4 region of the recombinant HXB2 gp120 envelope protein (SEQ ID NO:54) shown in FIGS. 10A-10F, and the N-terminus of the fourth subsequence is covalently linked by a peptide bond to the C-terminus of the gp120 V4 region sequence, and the C-terminus of the fourth subsequence is covalently linked by a peptide bond to the N-terminus of a gp120 V5 region sequence; and (e) the fifth subsequence of the gp120 variant sequence comprises a sequence corresponding by reference to amino acid residues 472-492 of the C5 region of the recombinant HXB2 gp120 envelope protein (SEQ ID NO:54) shown in FIGS. 10A-10F, and the N-terminus of the fifth subsequence is covalently linked by a peptide bond to the C-terminus of the V5 region sequence; wherein the gp120 V3 region sequence, the gp120 V4 region sequence and the gp120 V5 region sequence correspond by reference to the V3 region, the V4 region, and the V5 region, respectively, of the recombinant HXB2 gp120 envelope protein (SEQ ID NO:54) shown in FIGS. 10A-10F, and one or more of the gp120 V3 region sequence, the gp120 V4 region sequence and the gp120 V5 region sequence is not a subsequence of the selected gp120 variant sequence. In some instances, one or more of the gp120 V3 region sequence, the gp120 V4 region sequence, and the gp120 V5 region sequence is a subsequence of (i) the amino acid sequence of a gp120 variant selected from the group consisting of SEQ ID NOS:1-21 and SEQ ID NOS:56-63 excluding the selected amino acid sequence or (ii) the gp120 amino acid sequence of an HIV-1 viral strain, which subsequence corresponds by reference to the V3 region, the V4 region, or the V5 region, respectively, of the recombinant HXB2 gp120 envelope protein (SEQ ID NO:54) shown in FIGS. 10A-10F, wherein said one or more gp120 V3 region sequence, gp120 V4 region sequence, or gp120 V5 region sequence is not identical to the gp120 V3 region sequence, gp120 V4 region sequence, or gp120 V5 region sequence, respectively, of the selected amino acid sequence. In some such instances, two or three of the gp120 V3 region sequence, the gp120 V4 region sequence, and the gp120 V5 region sequence are subsequences of gp120 amino acid sequences of different HIV-1 viral strains, such as different HIV-1 subtype strains, e.g., different HIV-1 subtype B strains. For some such nucleic acids, the immune response comprises an anti-HIV-1 neutralizing antibody response or an HIV-specific T cell response or both. The immune response may be against at least two HIV-1 viruses or pseudoviruses of the same or different subtypes.

In yet another aspect, the invention provides an isolated or recombinant nucleic acid that induces in a subject to whom an effective amount of the nucleic acid is administered an immune response against at least one HIV-1 virus or HIV-1 pseudovirus, wherein said nucleic acid comprises a polynucleotide sequence having at least 85, 90, 91, 92, 93, 94, 95, 96, 97, 98, 99 or 100% sequence identity to an RNA polynucleotide sequence, said RNA polynucleotide sequence comprising a DNA sequence selected from the group of SEQ ID NOS:23-50 and 64-79 in which all of the thymine nucleotide residues in said DNA sequence are replaced or substituted with uracil nucleotide residues, or a complementary polynucleotide sequence thereof. Identity is calculated as if thymine residues are equivalent to uracil residues with respect to percent identity. Also provided is an RNA nucleic acid that hybridizes under at least stringent conditions over substantially the entire length of a nucleic acid comprising a nucleotide sequence having at least 80, 85, 90, 91, 92, 93, 94, 95, 96, 97, 98, 99, or 100% identity to a polynucleotide sequence selected from the group consisting of SEQ ID NOS:23-50 and 64-79, or that would so hybridize but for the degeneracy of the genetic code.

The invention also includes an isolated or recombinant nucleic acid encoding a polypeptide that has an ability to induce, promote, and/or enhance in a subject an immune response, wherein an amount effective to induce the immune response is administered, against at least one HIVs (e.g., HIV-1 virus or pseudovirus), wherein the nucleic acid comprises one or more of the following: (a) a polynucleotide sequence that encodes a polypeptide comprising an amino acid sequence having at least 80, 85, 90, 95, 96, 97, 98, 99, or 100% sequence identity to an amino acid sequence comprising of SEQ ID NO:1-21 and 56-63, or a complementary polynucleotide sequence thereof; (b) a polynucleotide sequence comprising nucleotide residues that encode a polypeptide that corresponds to (e.g., by alignment) to the HIV-1 HXB2 gp120 core polypeptide (SEQ ID NO:54), or a complementary polynucleotide sequence thereof; (c) a polynucleotide sequence selected from the group consisting of SEQ ID NOS:23-50 and 64-79, or a complementary polynucleotide sequence of any thereof; (d) a polynucleotide sequence having at least 85, 90, 91, 92, 93, 94, 95, 96, 97, 98, 99 or 100% sequence identity to an RNA polynucleotide sequence, said RNA polynucleotide sequence comprising a DNA sequence selected from the group of SEQ ID NOS:23-50 and 64-79 in which all of the thymine nucleotide residues in said DNA sequence are replaced with uracil nucleotide residues, or a complementary polynucleotide sequence thereof; and (e) a polynucleotide sequence that, but for the degeneracy of the genetic code, hybridizes under at least stringent conditions over substantially the entire length of the polynucleotide sequence of (a), (b), or (c) above. Such nucleic acid may encode at least one polypeptide that induces in the subject an immune response against one or more HIV-1 viruses, including against 1, 2, 3, 4, 5, 6, 7, 8, 9, or 10 HIV-1 viruses or pseudoviruses of the same or different subtypes or clades, or any combination of subtypes or clades. Some such nucleic acids encode at least one polypeptide that induce in the subject a neutralizing antibody immune response against one or more HIV-1 viruses, such as against 1, 2, 3, 4, 5, 6, 7, 8, 9, or 10 HIV-1 viruses or pseudoviruses of the same or different subtypes or any combination of subtypes. The neutralizing antibody response may be against 1, 2, 3, 4, 5, 6, 7, 8, 9, or 10 subtypes.

The invention also includes an isolated or recombinant nucleic acid comprising a polynucleotide sequence encoding a polypeptide comprising a fragment of a gp120 full-length polypeptide of the invention (e.g., any of SEQ ID NOS:1-7 and 56-63), wherein the fragment is a core polypeptide construct that binds to an HIV neutralizing antibody and/or induces in a subject to whom an effective amount of it is administered a neutralizing antibody response and/or T cell response against HIV-1. A core polypeptide of any of the gp120 full-length polypeptides of the invention (e.g., SEQ ID NOS:1-7 and 56-63) can be determined by comparison with other core polypeptides of the invention. For example, a core polypeptide of ST-008 (SEQ ID NO:1) comprises those amino acid residues of ST-008 that correspond by alignment to the amino acid residues of a gp120 core polypeptide variant of the invention, such as L7-068 (SEQ ID NO:11), L7-043 (SEQ ID NO:10), etc. (see FIGS. 10A-10F and FIG. 23A). The core polypeptide of ST-008 is identified herein as SEQ ID NO:109. A nucleic acid comprising the complementary sequence of said polynucleotide sequence is also included.

The invention also includes an isolated or recombinant nucleic acid comprising a polynucleotide sequence encoding a polypeptide comprising a fragment of a gp120 full-length polypeptide of the invention (e.g., any of SEQ ID NOS:1-7 and 56-63), wherein the fragment is a core+V1V2 polypeptide construct that binds to an HIV neutralizing antibody and/or induces in a subject to whom an effective amount of it is administered a neutralizing antibody response and/or T cell response against HIV-1. A core+V1V2 polypeptide of any of the gp120 full-length polypeptides of the invention (e.g., SEQ ID NOS:1-7 and 56-63) can be determined by comparison with other core+V1V2 polypeptides of the invention, such as the core+V1V2 polypeptide fragment of ST-008 (SEQ ID NO:1) which is identified herein as SEQ ID NO:110 (see also FIG. 23A). A nucleic acid comprising the complementary sequence of said polynucleotide sequence is also included.

The invention further provides an isolated or recombinant nucleic acid comprising a polynucleotide sequence encoding a polypeptide comprising a polypeptide fragment of a gp120 full-length polypeptide of the invention (e.g., any of SEQ ID NOS:1-7 and 56-63), wherein the fragment is a gp120ΔV1V2V3 polypeptide construct that binds to an HIV neutralizing antibody and/or induces in a subject to whom an effective amount of it is administered a neutralizing antibody response and/or T cell response against HIV-1. A gp120ΔV1V2V3 polypeptide of any of the gp120 full-length polypeptides of the invention (e.g., SEQ ID NOS:1-7 and 56-63) can be determined by comparison with other gp120ΔV1V2V3 polypeptides of the invention, such as the gp120ΔV1V2V3 polypeptide fragment of ST-008 (SEQ ID NO:1) identified herein as SEQ ID NO:108 (see also FIG. 23A). A nucleic acid comprising the complementary sequence of said polynucleotide sequence is also included.

The invention also includes an isolated or recombinant nucleic acid comprising a polynucleotide sequence encoding a polypeptide comprising a fragment of a gp120 full-length polypeptide of the invention (e.g., any of SEQ ID NOS:1-7 and 56-63), wherein the fragment is a gp120ΔV3 polypeptide construct that binds to an HIV neutralizing antibody and/or induces in a subject to whom an effective amount of it is administered a neutralizing antibody response and/or T cell response against HIV-1. A gp120ΔV3 polypeptide of any of the gp120 full-length polypeptides of the invention (e.g., SEQ ID NOS:1-7 and 56-63) can be determined by comparison with other gp120ΔV3 polypeptides of the invention, such as the gp120ΔV3 polypeptide of ST-008 (SEQ ID NO:1) identified herein as SEQ ID NO:107 (see also FIG. 23A). A nucleic acid comprising the complementary sequence of said polynucleotide sequence is also included.

Immune responses induced against HIV viruses or pseudoviruses by polypeptides encoded by nucleic acids of the invention may comprise a specific antibody response against one or more HIV-1 viruses or pseudoviruses derived from an HIV-1 virus (including, e.g., cross-reactive HIV-1 neutralizing antibody response); a T cell immune proliferation or activation response against one or more HIV-1 viruses (including, e.g., cross-reactive T cell response); an the ability to induce production of antibodies capable of specifically binding two or more HIV-1 viruses of the same or different subtypes (including, e.g., a cross-reactive Ab binding response); and/or the ability to induce or enhance production of other immunomodulatory molecules. For a detailed description regarding how to make a pseudovirus and how to conduct an HIV-1 neutralization assay for identifying/testing nucleic acids and/or polypeptide of the invention, see Richman et al., Proc. Natl. Acad. Sci. USA 100(7):4144-4149 (2003). Some such nucleic acids of the invention encode polypeptides that are capable of inducing an immune response against HIV that is about at least as great as the immune response induced by a WT HIV virus.

Many fragments of these nucleic acids will express polypeptides that induce one or more such immune responses, which can be readily identified with reasonable experimentation. Nucleotide fragments typically comprise at least 500 nucleotide bases, usually at least 600, 650, 700, 800, 900, 1000, 1200, 1300, 1400, 1500 or more bases. The nucleotide fragments, variants, analogs, and homologue derivatives of gp120 polypeptide variant-encoding polynucleotides may have hybridize under highly stringent conditions to another gp120 polypeptide variant-encoding polynucleotide or homologue sequence described herein and/or encode amino acid sequences having at least one of the anti-HIV immune response properties described herein.

A nucleic acid of the invention can further comprise one or more suitable additional nucleotide sequences. For example, given that a polypeptide of the invention can comprise one or more additional polypeptide sequences, such as, e.g., a polypeptide purification subsequence (such as, e.g., a subsequence is selected from an epitope tag, a FLAG tag, a polyhistidine sequence, and a GST fusion), signal peptide sequence, etc., the invention includes nucleic acids that encode all such polypeptides comprising such additional sequences. Exemplary signal peptides, which upon expression are typically covalently linked to the N-terminal of a polypeptide of the invention, are discussed above. For example, a nucleic acid encoding a polypeptide sequence of any of SEQ ID NOS:1-21 and 56-63 can further comprise a nucleic acid encoding a signal peptide, such as the signal peptide sequence of SEQ ID NO:52 or 55, such as, e.g., the nucleotide sequence set forth in SEQ ID NO:53. Such nucleotide sequences can be directly fused together, in appropriate reading frame, such that the resulting nucleic acid comprises a nucleotide sequence encoding a signal peptide of the invention and a nucleotide sequence encoding a polypeptide of the invention.

A nucleic acid of the invention can be isolated by any suitable technique, of which several are known in the art. An isolated nucleic acid of the invention (e.g., a nucleic acid that is prepared in a host cell and subsequently substantially purified by any suitable nucleic acid purification technique) can be re-introduced into a host cell or re-introduced into a cellular or other biological environment or composition wherein it is no longer the dominant nucleic acid species and is no longer separated from other nucleic acids.

Nearly any isolated or recombinant nucleic acid of the invention can be inserted in or fused to a suitable larger nucleic acid molecule (including e.g., but not limited to, a chromosome, a plasmid, an expression vector or cassette, a viral genome, a gene sequence, a linear expression element, a bacterial genome, a plant genome, or an artificial chromosome, such as a mammalian artificial chromosome (MAC), or the yeast and bacterial counterparts thereof (i.e., a YAC or a BAC) to form a recombinant nucleic acid using standard techniques. As another example, an isolated nucleic acid of the invention can be fused to smaller nucleotide sequences, such as promoter sequences, immunostimulatory sequences, and/or sequences encoding other amino acids, such as other antigen epitopes and/or linker sequences to form a recombinant nucleic acid.

A recombinant or synthetic nucleic acid is typically generated by chemical synthesis techniques applied outside of the context of a host cell (e.g., a nucleic acid produced through PCR or chemical synthesis techniques, examples of which are described further herein).

Nucleic acids encoding polypeptides of the invention can have any suitable chemical composition that permits the expression of a polypeptide of the invention or other desired biological activity (e.g., hybridization with other nucleic acids). The polynucleotides of the invention can be in the form of RNA or in the form of DNA, and include mRNA, cRNA, recombinant or synthetic RNA and DNA, and cDNA. The nucleic acids of the invention are typically DNA molecules, and usually double-stranded DNA molecules. However, single-stranded DNA, single-stranded RNA, double-stranded RNA, and hybrid DNA/RNA nucleic acids or combinations thereof comprising any of the nucleotide sequences of the invention also are provided. A nucleic acid of the invention can include any suitable nucleotide base, base analog, and/or backbone (e.g., a backbone formed by, or including, a phosphothioate, rather than phosphodiester, linkage, e.g., DNA comprising a phosphorothioate backbone). A nucleic acid of the invention, if single-stranded, can be the coding strand or the non-coding (i.e., antisense or complementary) strand. In addition to a nucleotide sequence encoding a polypeptide of the invention (e.g., nucleotide sequence that comprise the coding sequence of a gp120 polypeptide variant), the polynucleotide of the invention can comprise one or more additional coding nucleotide sequences, so as to encode, e.g., a fusion protein, targeting sequence (other than a signal sequence), or the like (more particular examples of which are discussed further herein), and/or can comprise non-coding nucleotide sequences, such as introns, terminator sequence, or 5′ and/or 3′ untranslated regions, which regions can be effective for expression of the coding sequence in a suitable host, and/or control elements, such as a promoter (e.g., naturally occurring or recombinant or shuffled promoter).

Modifications to a nucleic acid are particularly tolerable in the 3^{rd} position of an mRNA codon sequence encoding such a polypeptide. In particular aspects, at least a portion of the nucleic acid comprises a phosphorothioate backbone, incorporating at least one synthetic nucleotide analog in place of or in addition to the naturally occurring nucleotides in the nucleic acid sequence. Also or alternatively, the nucleic acid can comprise the addition of bases other than guanine, adenine, uracil, thymine, and cytosine. Such modifications can be associated with longer half-life, and thus can be desirable in nucleic acids vectors of the invention. Thus, in one aspect, the invention provides recombinant nucleic acids and nucleic acid vectors (discussed further below), which nucleic acids or vectors comprise at least one of the aforementioned modifications, or any suitable combination thereof, wherein the nucleic acid persists longer in a mammalian host than a substantially identical nucleic acid without such a modification or modifications. Examples of modified and/or non-cytosine, non-adenine, non-guanine, non-thymine nucleotides that can be incorporated in a nucleotide sequence of the invention are provided in, e.g., the MANUAL OF PATENT EXAMINING PROCEDURE §2422 (7^{th} Revision—2000).

It is to be understood that a nucleic acid encoding at least one of the polypeptides of the invention, including those described above and elsewhere herein, is not limited to a sequence that directly codes for expression or production of a polypeptide of the invention. For example, the nucleic acid can comprise a nucleotide sequence which results in a polypeptide of the invention through intein-like expression (as described in, e.g., Colson and Davis (1994) Mol. Microbiol. 12(3):959-63, Duan et al. (1997) Cell 89(4):555-64, Perler (1998) Cell 92(1):1-4, Evans et al. (1999) Biopolymers 51(5):333-42, and de Grey, Trends Biotechnol. 18(9):394-99 (2000)), or a nucleotide sequence which comprises self-splicing introns (or other self-spliced RNA transcripts), which form an intermediate recombinant polypeptide-encoding sequence (as described in, e.g., U.S. Pat. No. 6,010,884). The nucleic acid also or alternatively can comprise sequences which result in other splice modifications at the RNA level to produce an mRNA transcript encoding the polypeptide and/or at the DNA level by way of trans-splicing mechanisms prior to transcription (principles related to such mechanisms are described in, e.g., Chabot, Trends Genet. (1996) 12(11):472-78, Cooper (1997) Am. J. Hum. Genet. 61(2):259-66, and Hertel et al. (1997) Curr. Opin. Cell. Biol. 9(3):350-57). Due to the inherent degeneracy of the genetic code, several nucleic acids can code for any particular polypeptide of the invention. Thus, for example, any of the particular nucleic acids described herein can be modified by replacement of one or more codons with an equivalent codon (with respect to the amino acid called for by the codon) based on genetic code degeneracy. Other nucleotide sequences that encode a polypeptide having the same or a functionally equivalent sequence as a polypeptide sequence of the invention can also be used to synthesize, clone and express such polypeptide.

In general, any of the nucleic acids of the invention can be modified to increase expression in a particular host, using the techniques exemplified herein with respect to the above-described nucleic acids encoding a polypeptide of the invention (e.g., gp120 polypeptide variant-encoding sequences). Any of the nucleic acids of the invention as described herein may be codon optimized for expression in a particular mammal (normally humans). A variety of techniques for codon optimization are known in the art. Codons that are utilized most often in a particular host are called optimal codons, and those not utilized very often are classified as rare or low-usage codons (see, e.g., Zhang, S. P. et al. (1991) Gene 105:61-72). Codons can be substituted to reflect the preferred codon usage of the host, a process called “codon optimization” or “controlling for species codon bias.” Optimized coding sequence comprising codons preferred by a particular prokaryotic or eukaryotic host can be used to increase the rate of translation or to produce recombinant RNA transcripts having desirable properties, such as a longer half-life, as compared with transcripts produced from a non-optimized sequence. Techniques for producing codon-optimized sequences are known (see, e.g., E. et al. (1989) Nuc. Acids Res. 17:477-508). Translation stop codons can also be modified to reflect host preference. For example, preferred stop codons for S. cerevisiae and mammals are UAA and UGA respectively. The preferred stop codon for monocotyledonous plants is UGA, whereas insects and E. coli prefer to use UAA as the stop codon (see, e.g., Dalphin, M. E. et al. (1996) Nuc. Acids Res. 24:216-218). The arrangement of codons in context to other codons also can influence biological properties of a nucleic acid sequences, and modifications of nucleic acids to provide a codon context arrangement common for a particular host also is contemplated by the inventors. Thus, a nucleic acid sequence of the invention can comprise a codon optimized nucleotide sequence, i.e., codon frequency optimized and/or codon pair (i.e., codon context) optimized for a particular species (e.g., the polypeptide can be expressed from a polynucleotide sequence optimized for expression in humans by replacement of “rare” human codons based on codon frequency, or codon context, such as by using techniques such as those described in Buckingham et al. (1994) Biochimie 76(5):351-54 and U.S. Pat. Nos. 5,082,767, 5,786,464, and 6,114,148). Exemplary techniques for producing codon-optimized nucleic acid sequences is provided in Examples 2 and 7 below.

Nucleic acids of the invention can optionally comprise additional immunogenic acid sequences of the invention as described elsewhere herein. Further, nucleic acids can be modified by truncation or one or more residues of the C-terminus portion of the sequence. Additional, a variety of stop or termination codons may be included at the end of the nucleotide sequence as further discussed below.

One or more nucleic acids of the invention may be included in a vector, cell, or host environment in which a coding nucleotide sequence of the invention is a heterologous gene.

Polynucleotides of the invention include polynucleotide sequences that encode gp120 full-length polypeptide variants and gp120 core polypeptide variants, and fragments thereof that induce at least one immune response in a subject to whom said fragment(s) is administered in an amount effective to induce the immune response, polynucleotides that hybridize under at least stringent conditions to one or more polypeptide sequences defined herein, polynucleotide sequences complementary to these polynucleotide sequences, and variants, analogs, and homologue derivatives of all of the above. A coding sequence refers to a nucleotide sequence encodes a particular polypeptide or a domain, subsequence, region, or fragment of said polypeptide. A coding sequence may code for a gp120 polypeptide or fragment thereof having a functional property, such as the ability to induce an immune response against HIV.

In a particular aspect, a nucleic acid can comprise untranslated sequences associated with wild-type HIV gp120 polypeptide-encoding nucleic acid. The nucleic acid can be linked to the polyA sequence. Alternatively or additionally, the sequence can be associated with the GC rich noncoding sequences of gp120 and/or gp120 intron sequences.

A nucleic acid of the invention may comprise a respective coding sequence of a gp120 full-length or core polypeptide variant, and variants, analogs, and homologue derivatives thereof.

Nucleic acids of the invention can also be found in combination with typical compositional formulations of nucleic acids, including in the presence of carriers, buffers, adjuvants, excipients, and the like, as are known to those of ordinary skill in the art.

Unless otherwise indicated, a particular nucleic acid sequence described herein also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucl. Acid Res. 19:5081; Ohtsuka et al. (1985) J. Biol. Chem. 260:2605-2608; Cassol et al. (1992); Rossolini et al. (1994) Mol. Cell. Probes 8:91-98).

Nucleic Acid Hybridization

As noted above, the invention includes nucleic acids that hybridize to a target nucleic acid of the invention, such as, e.g. a polynucleotide selected from the group consisting of SEQ ID NOS:23-50 and 64-79, or a complementary polynucleotide sequence thereof, wherein hybridization is over substantially the entire length of the target nucleic acid. The hybridizing nucleic acid may hybridize to a nucleotide sequence of the invention, such as, e.g., that of SEQ ID NO:23, under at least stringent conditions or under at least high stringency conditions. Moderately stringent, stringent, and highly stringent hybridization conditions for nucleic acid hybridization experiments are known. Examples of factors that can be combined to achieve such levels of stringency are briefly discussed herein.

Nucleic acids “hybridize” when they associate, typically in solution. Nucleic acids hybridize due to a variety of well-characterized physico-chemical forces, such as hydrogen bonding, solvent exclusion, base stacking and the like. An extensive guide to the hybridization of nucleic acids is found in Tjissen (1993) LABORATORY TECHNIQUES IN BIOCHEMISTRY AND MOLECULAR BIOLOGY-HYBRIDIZATION WITH NUCLEIC ACID PROBES, part I, chapter 2, “Overview of principles of hybridization and the strategy of nucleic acid probe assays,” (Elsevier, New York) (hereinafter “Tjissen”), as well as in Ausubel, supra, Hames and Higgins (1995) GENE PROBES 1, IRL Press at Oxford University Press, Oxford, England (Hames and Higgins 1) and Hames and Higgins (1995) GENE PROBES 2, IRL Press at Oxford University Press, Oxford, England (Hames and Higgins 2) provide details on the synthesis, labeling, detection and quantification of DNA and RNA, including oligonucleotides.

An indication that two nucleic acid sequences are substantially identical is that the two molecules hybridize to each other under at least stringent conditions. The phrase “hybridizing specifically to,” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization wash conditions” and “stringent hybridization conditions” in the context of nucleic acid hybridization experiments, such as Southern and northern hybridizations, are sequence dependent, and are different under different environmental parameters. An extensive guide to hybridization of nucleic acids is found in Tijssen (1993), supra, and in Hames and Higgins 1 and Hames and Higgins 2, supra.

Generally, high stringency conditions are selected such that hybridization occurs at about 5° C. or less than the thermal melting point I for the specific sequence at a defined ionic strength and pH. The Tₘ is the temperature (under defined ionic strength and pH) at which 50% of the test sequence hybridizes to a perfectly matched probe. In other words, the Tₘ indicates the temperature at which the nucleic acid duplex is 50% denatured under the given conditions and its represents a direct measure of the stability of the nucleic acid hybrid. Thus, the Tₘ corresponds to the temperature corresponding to the midpoint in transition from helix to random coil; it depends on length, nucleotide composition, and ionic strength for long stretches of nucleotides. Typically, under “stringent conditions,” a probe will hybridize to its target subsequence, but to no other sequences. “Very stringent conditions” are selected to be equal to the Tₘ for a particular probe.

The Tₘ of a DNA-DNA duplex can be estimated using equation (1): Tₘ (° C.)=81.5° C.+16.6 (log₁₀M)+0.41 (% G+C)−0.72 (% f)−500/n, where M is the molarity of the monovalent cations (usually Na+), (% G+C) is the percentage of guanosine (G) and cytosine (C) nucleotides, (% f) is the percentage of formalize and n is the number of nucleotide bases (i.e., length) of the hybrid. See Rapley, R. and Walker, J. M. eds., MOLECULAR BIOMETHODS HANDBOOK (1998), Humana Press, Inc., Tijssen (1993) LABORATORY TECHNIQUES IN BIOCHEMISTRY AND MOLECULAR BIOLOGY-HYBRIDIZATION WITH NUCLEIC ACID PROBES. (hereinafter Rapley and Walker). The Tₘ of an RNA-DNA duplex can be estimated using equation (2): Tₘ (° C.)=79.8° C.+18.5 (log₁₀M)+0.58 (% G+C)−11.8 (% G+C)²−0.56 (% f)−820/n, where M is the molarity of the monovalent cations (usually Na+), (% G+C) is the percentage of guanosine (G) and cytosine (C) nucleotides, (% f) is the percentage of formamide and n is the number of nucleotide bases (i.e., length) of the hybrid. Id. Equations 1 and 2 above are typically accurate only for hybrid duplexes longer than about 100-200 nucleotides. Id. The Tₘ of nucleic acid sequences shorter than 50 nucleotides can be calculated as follows: Tₘ (° C.)=4(G+C)+2(A+T), where A (adenine), C, T (thymine), and G are the numbers of the corresponding nucleotides.

In general, non-hybridized nucleic acid material is removed by a series of washes, the stringency of which can be adjusted depending upon the desired results, in conducting hybridization analysis. Low stringency washing conditions (e.g., using higher salt and lower temperature) increase sensitivity, but can product nonspecific hybridization signals and high background signals. Higher stringency conditions (e.g., using lower salt and higher temperature that is closer to the hybridization temperature) lower the background signal, typically with only the specific signal remaining. Addition useful guidance concerning such hybridization techniques is provided in, e.g., Rapley and Walker, supra (in particular, with respect to such hybridization experiments, part I, chapter 2, “Overview of principles of hybridization and the strategy of nucleic acid probe assays”), Elsevier, New York, as well as in Ausubel, supra, Sambrook, supra, Watson, supra, Hames and Higgins (1995) GENE PROBES 1, IRL Press at Oxford Univ. Press, Oxford, England, and Hames and Higgins (1995) GENE PROBES 2, IRL Press, Oxford Univ. Press, Oxford, England.

Exemplary stringent (or regular stringency) conditions for analysis of at least two nucleic acids comprising at least 100 nucleotides include incubation in a solution or on a filter in a Southern or northern blot comprises 50% formalin (or formamide) with 1 milligram (mg) of heparin at 42° C., with the hybridization being carried out overnight. A regular stringency wash can be carried out using, e.g., a solution comprising 0.2×SSC wash at about 65° C. for about 15 minutes (see Sambrook, supra, for a description of SSC buffer). Often, the regular stringency wash is preceded by a low stringency wash to remove background probe signal. A low stringency wash can be carried out in, for example, a solution comprising 2×SSC at about 40° C. for about 15 minutes. A highly stringent wash can be carried out using a solution comprising 0.15 M NaCl at about 72° C. for about 15 minutes. An example medium (regular) stringency wash, less stringent than the regular stringency wash described above, for a duplex of, e.g., more than 100 nucleotides, can be carried out in a solution comprising 1×SSC at 45° C. for 15 minutes. An example low stringency wash for a duplex of, e.g., more than 100 nucleotides, is carried out in a solution of 4-6×SSC at 40° C. for 15 minutes. For short probes (e.g., about 10 to 50 nucleotides), stringent conditions typically involve salt concentrations of less than about 1.0 M Na⁺ ion, typically about 0.01 to 1.0 M Na⁺ ion concentration (or other salts) at pH 7.0 to 8.3, and the temperature is typically at least about 30° C. Stringent conditions can also be achieved with the addition of destabilizing agents such as formamide.

Exemplary moderate stringency conditions include overnight incubation at 37° C. in a solution comprising 20% formalin (or formamide), 0.5×SSC, 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook, supra, and/or Ausubel, supra.

High stringency conditions are conditions that use, for example, (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) at 55° C. in 50% formamide and (iii) at 55° C. in 0.1×SSC (preferably in combination with EDTA).

In general, a signal to noise ratio of 2× or 2.5×−5× (or higher) than that observed for an unrelated probe in the particular hybridization assay indicates detection of a specific hybridization. Detection of at least stringent hybridization between two sequences in the context of the present invention indicates relatively strong structural similarity or homology to, e.g., the nucleic acids of the present invention.

As noted, “highly stringent” conditions are selected to be about 5° C. or less lower than the thermal melting point I for the specific sequence at a defined ionic strength and pH. Target sequences that are closely related or identical to the nucleotide sequence of interest (e.g., “probe”) can be identified under highly stringency conditions. Lower stringency conditions are appropriate for sequences that are less complementary. See, e.g., Rapley and Walker; Sambrook, all supra.

Comparative hybridization can be used to identify nucleic acids of the invention, and this comparative hybridization method is a preferred method of distinguishing nucleic acids of the invention. Detection of highly stringent hybridization between two nucleotide sequences in the context of the present invention indicates relatively strong structural similarity/homology to, e.g., the nucleic acids provided in the sequence listing herein. Highly stringent hybridization between two nucleotide sequences demonstrates a degree of similarity or homology of structure, nucleotide base composition, arrangement or order that is greater than that detected by stringent hybridization conditions. In particular, detection of highly stringent hybridization in the context of the present invention indicates strong structural similarity or structural homology (e.g., nucleotide structure, base composition, arrangement or order) to, e.g., the nucleic acids provided in the sequence listing herein. For example, it is desirable to identify test nucleic acids that hybridize to the exemplar nucleic acids herein under stringent conditions.

Thus, one measure of stringent hybridization is the ability to hybridize to a nucleic acid of the invention (e.g., a nucleic acid comprising a polynucleotide sequence selected from the group consisting of SEQ ID NOS:23-50 and 64-79, or a complementary polynucleotide sequence thereof) under highly stringent conditions (or very stringent conditions, or ultra-high stringency hybridization conditions, or ultra-ultra high stringency hybridization conditions). Stringent hybridization (including, e.g., highly stringent, ultra-high stringency, or ultra-ultra high stringency hybridization conditions) and wash conditions can easily be determined empirically for any test nucleic acid.

For example, in determining highly stringent hybridization and wash conditions, the hybridization and wash conditions are gradually increased (e.g., by increasing temperature, decreasing salt concentration, increasing detergent concentration and/or increasing the concentration of organic solvents, such as formalin, in the hybridization or wash), until a selected set of criteria is met. For example, the hybridization and wash conditions are gradually increased until a probe comprising one or more nucleic acid sequences selected from the group consisting of SEQ ID NOS:23-50 and 64-79, and complementary polynucleotide sequences thereof, binds to a perfectly matched complementary target (again, a nucleic acid comprising one or more nucleic acid sequences selected from the group consisting of SEQ ID NOS:23-50 and 64-79, and complementary polynucleotide sequences thereof), with a signal to noise ratio that is at least 2.5×, and optionally 5× or more as high as that observed for hybridization of the probe to an unmatched target. The unmatched target may comprise a nucleic acid corresponding to, e.g., an HIV-1 gp120 nucleic acid sequence.

Preferably, the hybridization analysis is carried out under hybridization conditions selected such that a nucleic acid comprising a sequence that is perfectly complementary to the a disclosed reference (or known) nucleotide sequence (e.g., SEQ ID NO:23) hybridizes with the recombinant antigen-encoding sequence (e.g., a nucleotide sequence variant of the nucleic acid sequence of SEQ ID NO:23) with at least about 5, 7, or 10 times higher signal-to-noise ratio than is observed in the hybridization of the perfectly complementary nucleic acid to a nucleic acid that comprises a nucleotide sequence that is at least about 80 or 90% identical to the reference nucleic acid. Such conditions can be considered indicative for specific hybridization.

The above-described hybridization conditions can be adjusted, or alternative hybridization conditions selected, to achieve any desired level of stringency in selection of a hybridizing nucleic acid sequence. For example, the above-described highly stringent hybridization and wash conditions can be gradually increased (e.g., by increasing temperature, decreasing salt concentration, increasing detergent concentration and/or increasing the concentration of organic solvents, such as formalin, in the hybridization or wash), until a selected set of criteria are met. For example, the hybridization and wash conditions can be gradually increased until a desired probe, binds to a matched complementary target, with a signal-to-noise ratio that is at least about 2.5×, and optionally at least about 5× (e.g., about 10×, about 20×, about 50×, about 100×, or even about 500×), as high as the signal-to-noise ration observed from hybridization of the probe to a nucleic acid not of the invention, such as a wild-type HIV-1 gp120 polypeptide-encoding DNA sequence (e.g., JRCSF gp120 full-length polypeptide-encoding DNA sequence or JRCSF gp120 core polypeptide-encoding DNA sequence).

Making and Modifying Nucleic Acids

Nucleic acids of the invention can be obtained and/or generated by application of any suitable synthesis, manipulation, and/or isolation techniques, or combinations thereof. Exemplary procedures are described infra. For example, polynucleotides of the invention are typically produced through standard nucleic acid synthesis techniques, such as solid-phase synthesis techniques known in the art. In such techniques, fragments of up to about 100 bases usually are individually synthesized, then joined (e.g., by enzymatic or chemical ligation methods, or polymerase mediated recombination methods) to form essentially any desired continuous nucleic acid sequence. The synthesis of the nucleic acids of the invention can be also facilitated (or alternatively accomplished), by chemical synthesis using, e.g., the classical phosphoramidite method, which is described in, e.g., Beaucage et al. (1981) Tetrahedron Letters 22:1859-69, or the method described by Matthes et al. (1984) EMBO J. 3:801-05, e.g., as is typically practiced in automated synthetic methods. The nucleic acid of the invention also can be produced by use of an automatic DNA synthesizer. Other techniques for synthesizing nucleic acids and related principles are described in, e.g., Itakura et al., Annu. Rev. Biochem. 53:323 (1984), Itakura et al., Science 198:1056 (1984), and Ike et al., Nucl. Acid Res. 11:477 (1983).

Conveniently, custom made nucleic acids can be ordered from a variety of commercial sources, such as The Midland Certified Reagent Company (mcrc@oligos.com), the Great American Gene Company (world wide website address genco.com), ExpressGen Inc. (world wide website address expressgen.com), Operon Technologies Inc. (Alameda, Calif.). Similarly, custom peptides and antibodies can be custom ordered from any of a variety of sources, e.g., PeptidoGenic (pkim@ccnet.com), HTI Bio-products, Inc. (world wide website address htibio.com), and BMA Biomedicals Ltd. (U.K.), Bio.Synthesis, Inc.

Certain nucleotides of the invention may also be obtained by screening cDNA libraries using oligonucleotide probes that can hybridize to or PCR-amplify polynucleotides which encode the polypeptides of the invention. Procedures for screening and isolating cDNA clones are well-known to those of skill in the art; exemplary procedures are described infra. Such techniques are described in, e.g., Berger and Kimmel, “Guide to Molecular Cloning Techniques,” in Methods in Enzymol. Vol. 152, Acad. Press, Inc., San Diego, Calif. (“Berger”); Sambrook, supra, and CURRENT PROTOCOLS IN MOLECULAR BIOLOGY, Ausubel, supra. Some nucleic acids of the invention can be obtained by altering a naturally occurring backbone, e.g., by mutagenesis, in vitro recombination (e.g., shuffling), or oligonucleotide recombination. In other cases, such polynucleotides can be made in silico or through oligonucleotide recombination methods as described in the references cited herein.

Recombinant DNA techniques useful in modification of nucleic acids are well known in the art (e.g., restriction endonuclease digestion, ligation, reverse transcription and cDNA production, and PCR). Useful recombinant DNA technology techniques and principles related thereto are provided in, e.g., Mulligan (1993) Science 260:926-932, Friedman (1991) THERAPY FOR GENETIC DISEASES, Oxford University Press, Ibanez et al. (1991) EMBO J. 10:2105-10, Ibanez et al. (1992) Cell 69:329-41 (1992), and U.S. Pat. Nos. 4,440,859, 4,530,901, 4,582,800, 4,677,063, 4,678,751, 4,704,362, 4,710,463, 4,757,006, 4,766,075, and 4,810,648, and are more particularly described in Sambrook et al. (1989) MOLECULAR CLONING: A LABORATORY MANUAL, Cold Spring Harbor Press, and the third edition thereof (2001), Ausubel et al. (1994-1999), Current Protocols in Molecular Biology, Wiley Interscience Publishers (with Greene Publishing Associates for some editions), Berger, supra, and Watson, supra.

Substrates and Formats for Sequence Recombination and Mutagenesis

The polynucleotides of the invention and fragments thereof are optionally used as substrates for any of a variety of recombination methods, in addition to their use in standard cloning methods as set forth in, e.g., Ausubel, Berger, and Sambrook, supra, e.g., to produce additional gp120 polypeptide variant-encoding polynucleotides or fragment thereof, which encode gp120 polypeptide variants or fragments thereof having desired antigenic or immunogenic properties, such as those described herein.

A variety of protocols exist for generating and identifying molecules of the invention having one of more of the properties described herein. These procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics. While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity-generating procedures described herein can be the generation of one or more nucleic acids, which can be selected or screened for nucleic acids with or which confer desirable properties, or that encode proteins with or which confer desirable properties. Following diversification by one or more of the methods herein, or otherwise available to one of skill, any nucleic acids that are produced can be selected for a desired activity or property described herein, including, e.g., an ability to induce, promote, enhance, or modulate an immune response, favorably an immune response against HIV-1, such T cell proliferation and/or activation, cytokine production (e.g., (e.g., IL-3 production and/or IFN-γ production), and/or the production of antibodies that bind (react) with one or more HIV-1 viruses.

Descriptions of a variety of diversity generating procedures for generating modified nucleic acid sequences that encode polypeptides of the invention as described herein are found in the following publications and the references cited therein: Soong, N. et al. (2000) Nat Genet. 25(4):436-439; Stemmer et al. (1999) Tumor Targeting 4:1-4; Ness et al. (1999) Nature Biotechnol. 17:893-896; Chang et al. (1999) Nature Biotechnology 17:793-797; Minshull and Stemmer (1999) Curr. Opin. Chemical Biol. 3:284-290; Christians et al. (1999) Nature Biotechnol. 17:259-264; Crameri et al. (1998) Nature 391:288-291; Crameri et al. (1997) Nature Biotechnol. 15:436-438; Zhang et al. (1997) Proc. Natl. Acad. Sci. USA 94:4504-4509; Patten et al. (1997) Curr. Opin. Biotechnol. 8:724-733; Crameri et al. (1996) Nature Med. 2:100-103; Crameri et al. (1996) Nature Biotechnol. 14:315-319; Gates et al. (1996) J. Mol. Biol. 255:373-386; Stemmer (1996) “Sexual PCR and Assembly PCR” In: The Encyclopedia of Molecular Biology, VCH Publishers, NY pp. 447-457; Crameri and Stemmer (1995) BioTechniq. 18:194-195; Stemmer et al., (1995) Gene 164:49-53; Stemmer (1995) Science 270:1510; Stemmer (1995) Bio/Technology 13:549-553; Stemmer (1994) Nature 370:389-391; and Stemmer (1994) Proc. Natl. Acad. Sci. USA 91:10747-10751.

The term “shuffling” is used herein to indicate recombination between non-identical sequences, in some embodiments shuffling may include crossover via homologous recombination or via non-homologous recombination, such as via cre/lox and/or flp/frt systems. Shuffling can be carried out by employing a variety of different formats, including for example, in vitro and in vivo shuffling formats, in silico shuffling formats, shuffling formats that utilize either double-stranded or single-stranded templates, primer based shuffling formats, nucleic acid fragmentation-based shuffling formats, and oligonucleotide-mediated shuffling formats, all of which are based on recombination events between non-identical sequences and are described in more detail or referenced herein below, as well as other similar recombination-based formats.

DNA-based recombination can be used to generate and identify new polypeptides having (e.g., gp120 polypeptide variants), including those having an ability to induce HIV-1-specific immune responses as described herein.

Mutational methods of generating diversity include, for example, site-directed mutagenesis (Ling et al. (1997) Anal. Biochem. 254(2):157-178; Dale et al. (1996) Mol. Biol. 57:369-374; Smith (1985) Ann. Rev. Genet. 19:423-462; Botstein & Shortle (1985) Science 229:1193-1201; Carter (1986) Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) Proc. Natl. Acad. Sci. USA 82:488-492; Kunkel et al. (1987) Meth. Enzymol. 154, 367-382; and Bass et al. (1988) Science 242:240-245); oligonucleotide-directed mutagenesis (Methods in Enzymol. 100:468-500 (1983); Meth. Enzymol. 154:329-350 (1987); Zoller & Smith (1982) Nucl. Acids Res. 10:6487-6500; Zoller & Smith (1983) Meth. Enzymol. 100:468-500; and Zoller & Smith (1987) Meth. Enzymol. 154:329-350); phosphorothioate-modified DNA mutagenesis (Taylor et al. (1985) Nucl. Acids Res. 13:8749-8764; Taylor et al. (1985) Nucl. Acids Res. 13:8765-8787 (1985); Nakamaye & Eckstein (1986) Nucl. Acids Res. 14:9679-9698; Sayers et al. (1988) Nucl. Acids Res. 16:791-802; and Sayers et al. (1988) Nucl. Acids Res. 16:803-814); mutagenesis using gapped duplex DNA (Kramer et al. (1984) Nucl. Acids Res. 12:9441-9456; Kramer & Fritz (1987) Meth. Enzymol. 154:350-367; Kramer et al. (1988) Nucl. Acids Res. 16:7207; and Fritz et al. (1988) Nucl. Acids Res. 16:6987-6999).

Additional suitable diversity-generating methods include point mismatch repair (Kramer et al. (1984) Cell 38:879-887), mutagenesis using repair-deficient host strains (Carter et al. (1985) Nucl. Acids Res. 13:4431-4443; and Carter (1987) Meth. Enzymol. 154:382-403), deletion mutagenesis (Eghtedarzadeh & Henikoff (1986) Nucl. Acids Res. 14:5115), restriction-selection and restriction-purification (Wells et al. (1986) Phil. Trans. R. Soc. Lond. A 317:415-423), mutagenesis by total gene synthesis (Nambiar et al. (1984) Science 223:1299-1301; Sakamar and Khorana (1988) Nucl. Acids Res. 14:6361-6372; Wells et al. (1985) Gene 34:315-323; and Grundström et al. (1985) Nucl. Acids Res. 13:3305-3316), double-strand break repair (Mandecki (1986) Proc. Natl. Acad. Sci. USA 83:7177-7181; and Arnold (1993) Curr. Opin. Biotechnol. 4:450-455). Additional details on many of the above methods can be found in Methods in Enzymology Volume 154, which also describes useful controls for trouble-shooting problems with various mutagenesis methods.

Additional site-mutagenesis techniques are described in, e.g., Edelman et al., DNA 2:183 (1983), Zoller et al., Nucl. Acids Res. 10:6487-5400 (1982), Veira et al., Meth. Enzymol. 153:3 (1987)). Other useful mutagenesis techniques include alanine scanning, or random mutagenesis, such as iterated random point mutagenesis induced by error-prone PCR, chemical mutagen exposure, or polynucleotide expression in mutator cells (see, e.g., Bornscheueret et al., Biotechnol. Bioeng. 58, 554-59 (1998), Cadwell and Joyce, PCR Methods Appl. 3(6):S136-40 (1994), Kunkel et al., Meth. Enzymol. 204:125-39 (1991), Low et al., J. Mol. Biol. 260:359-68 (1996), Taguchi et al., Appl. Environ. Microbiol. 64(2): 492-95 (1998), and Zhao et al., Nat. Biotech. 16:258-61 (1998) for discussion of such techniques). Suitable primers for PCR-based site-directed mutagenesis or related techniques can be prepared by methods described in Crea et al., Proc. Natl. Acad. Sci. USA 75:5765 (1978).

Other useful techniques for promoting sequence diversity include PCR mutagenesis techniques (as described in, e.g., Kirsch et al., Nucl. Acids Res. 26(7):1848-50 (1998), Seraphin et al., Nucl. Acids Res. 24(16):3276-7 (1996), Caldwell et al., PCR Methods Appl. 2(1):28-33 (1992), Rice et al., Proc. Natl. Acad. Sci. USA. 89(12):5467-71 (1992) and U.S. Pat. No. 5,512,463), cassette mutagenesis techniques based on the methods described in Wells et al., Gene 34:315 (1985), phagemid display techniques (as described in, e.g., Soumillion et al., Appl. Biochem. Biotechnol. 47:175-89 (1994), O'Neil et al., Curr. Opin. Struct. Biol. 5(4):443-49 (1995), Dunn, Curr. Opin. Biotechnol. 7(5):547-53 (1996), and Koivunen et al., J. Nucl. Med. 40(5):883-88 (1999)), reverse translation evolution (as described in, e.g., U.S. Pat. No. 6,194,550), saturation mutagenesis described in, e.g., U.S. Pat. No. 6,171,820), PCR-based synthesis shuffling (as described in, e.g., U.S. Pat. No. 5,965,408) and recursive ensemble mutagenesis (REM) (as described in, e.g., Arkin and Yourvan, Proc. Natl. Acad. Sci. USA 89:7811-15 (1992), and Delgrave et al., Protein Eng. 6(3):327-331 (1993)). Techniques for introducing diversity into a library of homologous sequences also are provided in U.S. Pat. Nos. 6,159,687 and 6,228,639.

Further details regarding various diversity generating methods can be found in the following U.S. patents, PCT publications and applications, and European publications: U.S. Pat. Nos. 5,605,793, 5,811,238, 5,830,721, 5,834,252, 5,837,458, and Int'l Pat. Appn. Publication Nos. WO 95/22625, WO 96/33207, WO 97/20078, WO 97/35966, WO 99/41402, WO 99/41383, WO 99/41368, WO 99/23107, WO 99/21979, WO 98/31837, WO 98/27230, WO 98/27230, WO 00/00632, WO 00/09679, WO 98/42832, WO 99/29902, WO 98/41653, WO 98/41622, WO 98/42727, WO 00/18906, WO 00/04190, WO 00/42561, WO 00/42559, WO 00/42560, PCT/US00/26708, PCT/US01/06775, and European Pat. Appn. Nos. EP 752008, EP 0932670.

Several different general classes of sequence modification methods, such as mutation, recombination, etc. are applicable to the present invention and set forth, e.g., in the references above and below. That is, nucleic acids encoding polypeptides having the desired activities or properties (e.g., such as an ability to enhance an immune response against an HIV virus) can be diversified by any of the methods described herein, e.g., including various mutation and recombination methods, individually or in combination, to generate nucleic acids with a desired activity or property, including, e.g., those described herein. The following exemplify some of the different types of formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 and PCT/U599/15972.

Synthetic recombination methods can also be used in which oligonucleotides corresponding to targets of interest (e.g., gp120 polypeptide antigens) are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561; PCT/US00/26708; WO 00/42560; and WO 00/42559.

In silico methods of recombination can be effected in which genetic algorithms are used in a computer to recombine sequence strings that correspond to homologous (or even non-homologous) nucleic acids. The resulting recombined sequence strings are optionally converted into nucleic acids by synthesis of nucleic acids that correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 and WO 00/42559. Extensive details regarding in silico recombination methods are found in these applications. This methodology is generally applicable to the nucleic acid sequences and polypeptide sequences of the invention.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full-length single-stranded DNA or RNA corresponding to the opposite strand. Assembly of complex chimeric genes from this population is then mediated by nuclease-base removal of non-hybridizing fragment ends, polymerization to fill gaps between such fragments and subsequent single-stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate library-enriched sequences, which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see, e.g., U.S. Pat. No. 5,965,408 and the references above), and can be applied to the present invention. In this approach, double-stranded DNAs corresponding to one or more genes sharing regions of sequence similarity are combined and denatured, in the presence or absence of primers specific for the gene. The single-stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., comprising partially extended chains, are then denatured and re-annealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombination procedure known as “incremental truncation for the creation of hybrid enzymes” (“ITCHY”) described in Ostermeier et al. (1999) Nature Biotech 17:1205. This approach can be used to generate an initial a library of variants, which can optionally serve as a substrate for one or more in vitro or in vivo recombination methods. See also Ostermeier et al. (1999) Proc. Natl. Acad. Sci. USA 96:3562-67; Ostermeier et al. (1999), Biological and Medicinal Chemistry 7:2139-44.

Mutational methods that result in the alteration of individual nucleotides or groups of contiguous or non-contiguous nucleotides can be favorably employed to introduce nucleotide diversity. Many mutagenesis methods are found in the above-cited references; additional details regarding mutagenesis methods can be found in following, which can also be applied to the present invention. For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double-stranded DNA molecule with a synthetic oligonucleotide cassette that differs from the native sequence. The oligonucleotide can include, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above. Alternatively, in vivo recombination techniques can be used. For example, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above. Other techniques that can be used for in vivo recombination and sequence diversification are described in U.S. Pat. No. 5,756,316.

Methods for generating multispecies expression libraries have been described (in addition to the reference noted above, see, e.g., U.S. Pat. Nos. 5,783,431 and 5,824,485 and their use to identify protein activities of interest has been proposed. In addition to the references noted above, see U.S. Pat. No. 5,958,672. Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

Nucleotide sequences of the present invention can be engineered by standard techniques to make additional modifications, such as, the insertion of new restriction sites, the alteration of glycosylation patterns, the alteration of PEGylation patterns, modification of the sequence based on host cell codon preference, the introduction of recombinase sites, and the introduction of splice sites.

In some applications, it is desirable to preselect or prescreen libraries (e.g., an amplified library, a cDNA library, a normalized library, etc.) or other substrate nucleic acids prior to diversification, e.g., by recombination-based mutagenesis procedures, or to otherwise bias the substrates towards nucleic acids that encode functional products. Libraries can also be biased towards nucleic acids that have specified characteristics, e.g., hybridization to a selected nucleic acid probe. For example, after identifying a clone from a library that exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in U.S. Pat. No. 5,939,250. Desired activities can be identified by any method known in the art. For example, WO 99/10539 proposes that gene libraries can be screened by combining extracts from the gene library with components obtained from metabolically rich cells and identifying combinations that exhibit the desired activity. It has also been proposed (e.g., WO 98/58085) that clones with desired activities can be identified by inserting bioactive substrates into samples of the library, and detecting bioactive fluorescence corresponding to the product of a desired activity as described herein using a fluorescent analyzer, e.g., a flow cytometry device, a CCD, a fluorometer, or a spectrophotometer.

Libraries can also be biased towards nucleic acids that have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single-stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides, including proposed non-stochastic polynucleotide reassembly and site-saturation mutagenesis methods, are applicable to the present invention as well. Random or semi-random mutagenesis using doped or degenerate oligonucleotides is also described in, e.g., Arkin and Youvan (1992) Biotechnol. 10:297-300; Reidhaar-Olson et al. (1991) Meth. Enzymol. 208:564-86; Lim and Sauer (1991) J. Mol. Biol. 219:359-76; Breyer and Sauer (1989) J. Biol. Chem. 264:13355-60); and U.S. Pat. Nos. 5,830,650 and 5,798,208, and European Patent No. 0 527 809B1.

It will readily be appreciated that any of the above-described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

A recombinant nucleic acid produced by recombining one or more polynucleotide sequences of the invention with one or more additional nucleic acids using any of the above-described formats alone or in combination also forms a part of the invention. The one or more additional nucleic acids may include another polynucleotide of the invention; or, e.g., any other homologous or non-homologous nucleic acid or fragments thereof (certain recombination formats noted above, notably those performed synthetically or in silico, do not require homology for recombination).

Polynucleotides of the invention, including those produced by the above-described recombination, mutagenesis, and standard nucleotide synthesis techniques described herein can be screened for any suitable characteristic, such as the expression of a recombinant polypeptide able to induce in a subject whom an effective amount of said polynucleotide(s) is administered an immune response against at least one HIV virus, e.g., HIV-1, or HIV pseudovirus. Polypeptides produced by such techniques and having such characteristics are an important feature of the invention. The invention includes a recombinant polypeptide encoded by a recombinant polynucleotide produced by an in vitro recombination method (e.g., shuffling) with any nucleic acid sequence of the invention that induces an immune response against one or more HIV viruses or pseudoviruses as described herein.

Modified Coding Sequences

Where appropriate, nucleic acids of the invention can be modified to increase or enhance expression in a particular host by modification of the sequence with respect to codon usage and/or codon context, given the particular host(s) in which expression of the nucleic acid is desired. Codons that are utilized most often in a particular host are called optimal codons, and those not utilized very often are classified as rare or low-usage codons (see, e.g., Zhang, S. P. et al. (1991) Gene 105:61-72). Codons can be substituted to reflect the preferred codon usage of the host, a process called “codon optimization” or “controlling for species codon bias”.

Optimized coding sequence comprising codons preferred by a particular prokaryotic or eukaryotic host can be used to increase the rate of translation or to produce recombinant RNA transcripts having desirable properties, such as a longer half-life, as compared with transcripts produced from a non-optimized sequence. Techniques for producing codon-optimized sequences are known (see, e.g., Murray, E. et al. (1989) Nucl. Acids Res. 17:477-508). Translation stop codons can also be modified to reflect host preference. For example, preferred stop codons for S. cerevisiae and mammals are UAA and UGA respectively. The preferred stop codon for monocotyledonous plants is UGA, whereas insects and E. coli prefer to use UAA as the stop codon (see, e.g., Dalphin, M. E. et al. (1996) Nucl. Acids Res. 24:216-218, for discussion). The arrangement of codons in context to other codons also can influence biological properties of a nucleic acid sequences, and modifications of nucleic acids to provide a codon context arrangement common for a particular host also is contemplated by the inventors. Thus, a nucleic acid sequence of the invention can comprise a codon optimized nucleotide sequence, i.e., codon frequency optimized and/or codon pair (i.e., codon context) optimized for a particular species (e.g., the polypeptide can be expressed from a polynucleotide sequence optimized for expression in humans by replacement of “rare” human codons based on codon frequency, or codon context, such as by using techniques such as those described in Buckingham et al. (1994) Biochimie 76(5):351-54 and U.S. Pat. Nos. 5,082,767, 5,786,464, and 6,114,148). For example, the invention provides a nucleic acid comprising a nucleotide sequence variant of SEQ ID NO:23, wherein the nucleotide sequence variant differs from SEQ ID NO:23 by the substitution of “rare” codons for a particular host with codons commonly expressed in the host, which codons encode the same amino acid residue as the substituted “rare” codons in SEQ ID NO:23.

Using Nucleic Acids

Nucleic acids of the invention and fragments thereof can be used as substrates for any of a variety of recombination methods described herein, in addition to their use in standard cloning methods as set forth in, e.g., Ausubel, Berger, and Sambrook, e.g., to produce additional polynucleotides or fragments thereof that encode recombinant antigens of the invention having desired properties. A variety of such reactions are known, including those developed by the inventors and their co-workers.

Nucleic acids of the invention, and nucleic acid vectors or other vectors described below comprising at least one nucleic acid of the invention, are also useful in a variety of prophylactic and/or therapeutic methods for inducing in a subject, including a human, to whom an effective amount of such polynucleotide is administered, an immune response to one or more HIV viruses as discussed in more detail below.

The nucleic acids of the invention also can be useful for sense and anti-sense suppression of expression (e.g., to regulate expression of a nucleic acid of the invention once or when expression is no longer require or to control nucleic acid expression levels in tissues away from those in which expression of an administered nucleic acid or vector is desired). A variety of sense and anti-sense technologies are known in the art, e.g., as set forth in Lichtenstein and Nellen (1997) ANTISENSE TECHNOLOGY: A PRACTICAL APPROACH IRL Press at Oxford University, Oxford, England, and in Agrawal (1996) ANTISENSE THERAPEUTICS, Humana Press, NJ, and the references cited therein.

In this respect, the invention provides nucleic acids that comprise a nucleic acid sequence that is the substantial complement (i.e., comprises a nucleotide sequence that complements at least about 90%, 95, 96, 97, 98, 99%), and the complement of any of the above-described nucleic acid sequences. Such complementary nucleic acid sequences are useful in probes, production of the nucleic acid sequences of the invention, and as antisense nucleic acids for hybridizing to nucleic acids of the invention. Short oligonucleotide sequences comprising sequences that complement the nucleic acid, e.g., of about 15, about 20, about 30, or about 50 bases (preferably at least about 12 bases), which hybridize under highly stringent conditions to a nucleic acid of the invention also are useful as probes (e.g., to determine the presence of a nucleic acid of the invention in a particular cell or tissue and/or to facilitate the purification of nucleic acids of the invention). The polynucleotides comprising complementary sequences also can be used as primers for amplification of the nucleic acids of the invention.

Additional uses of the nucleic acids and vectors of the invention are described elsewhere herein.

Vectors, Vector Components, and Expression Systems

The present invention also includes recombinant constructs comprising one or more of the nucleic acids of the invention as broadly described above. Such constructs may comprise a vector, such as a plasmid, a cosmid, a phage, a virus, a viral particle, a virus-like particle, a bacterial artificial chromosome (BAC), a yeast artificial chromosome (YAC), or the like, or a non-replicating vector, such as a liposome, naked or conjugated DNA, DNA-microparticle, into which at least one nucleic acid sequence of the invention has been inserted, in a forward or reverse orientation. In a particular aspect of this embodiment, the construct further comprises one or more regulatory sequences, including, for example, a promoter, operably linked to a nucleic acid sequence of the invention (e.g., nucleic acid encoding a recombinant gp120 polypeptide variant). Large numbers of suitable vectors and promoters are known to those of skill in the art and are commercially available. In some instances, a vector, such as, e.g., a virus or virus-like particle, may also or alternatively include one or more polypeptides of the invention such as, e.g., incorporated into the coat of the virus or virus-like particle. Vectors can be useful as delivery agents for the delivery or administration to a subject of exogenous genes or proteins. Vectors of the present invention, including those described herein, are useful as delivery agents for the delivery or administration of nucleic acids and/or polypeptides of the invention, such as, e.g., recombinant gp120 polypeptide variants and nucleic acids encoding such variants.

General texts that describe molecular biological techniques useful herein, including the use of vectors, promoters, and many other relevant topics, include Berger, supra, Sambrook (1989), supra, and Ausubel, supra. Examples of techniques sufficient to direct persons of skill through in vitro amplification methods, including the polymerase chain reaction (PCR) the ligase chain reaction (LCR), Q∃-replicase amplification and other RNA polymerase mediated techniques (e.g., NASBA), e.g., for the production of the homologous nucleic acids of the invention are found in Berger, Sambrook, and Ausubel, all supra, as well as Mullis et al. (1987) U.S. Pat. No. 4,683,202; PCR Protocols: A Guide to Methods and Applications (Innis et al., eds.) Academic Press Inc. San Diego, Calif. (1990) (“Innis”); Arnheim & Levinson (Oct. 1, 1990) C&EN 36-47; The Journal Of NIH Research (1991) 3:81-94; (Kwoh et al. (1989) Proc. Natl. Acad. Sci. USA 86:1173-1177; Guatelli et al. (1990) Proc. Natl. Acad. Sci. USA 87:1874-1878; Lomeli et al. (1989) J. Clin. Chem. 35:1826-1831; Landegren et al. (1988) Science 241:1077-1080; Van Brunt (1990) Biotechnology 8:291-294; Wu and Wallace (1989) Gene 4:560-569; Barringer et al. (1990) Gene 89:117-122, and Sooknanan and Malek (1995) Biotechnology 13:563-564.

PCR generally refers to a procedure wherein minute amounts of a specific piece of nucleic acid (e.g., RNA or DNA) are amplified by methods well known in the art (see, e.g., U.S. Pat. No. 4,683,195 and the other references cited above). Generally, sequence information from the ends of the region of interest or beyond is used for design of oligonucleotide primers. Such primers will be identical or similar in sequence to the opposite strands of the template to be amplified. The 5′ terminal nucleotides of the opposite strands may coincide with the ends of the amplified material. PCR may be used to amplify specific RNA or specific DNA sequences, recombinant DNA or RNA sequences, DNA and RNA sequences from total genomic DNA, and cDNA transcribed from total cellular RNA, bacteriophage or plasmid sequences, etc. PCR is one example, but not the only example, of a nucleic acid polymerase reaction method for amplifying a nucleic acid test sample comprising the use of another (e.g., known) nucleic acid as a primer. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684 685 and the references cited therein, in which PCR amplicons of up to 40 kilobases (kb) are generated. One of skill will appreciate that essentially any RNA can be converted into a double-stranded DNA suitable for restriction digestion, PCR expansion and sequencing using reverse transcriptase and a polymerase. See Ausubel, Sambrook and Berger, all supra.

The nucleic acids of the present invention can be incorporated into any one of a variety of vectors, e.g., expression vectors, for expressing a polypeptide, including, e.g., a polypeptide of the invention. Such vectors include chromosomal, nonchromosomal and synthetic DNA sequences, e.g., derivatives of SV40, bacterial vectors (e.g., S. typhimurium, S. typhi, S. flexneri, Listeria monocytogenes, B. anthracis); plasmids; bacterial plasmids; phage DNA; baculovirus; yeast plasmids; vectors derived from combinations of plasmids and phage DNA; viral DNA or RNA vectors, including, e.g., vaccinia virus, adeno-associated virus (AAV), adenovirus, Semliki-Forest virus (e.g., Notka et al., Biol. Chem. 380:341-52 (1999), pox virus (e.g., MVA), alphavirus (e.g., Venezuelan equine encephalitis virus (VEE), Western equine encephalitis virus (WEE), Eastern equine encephalitis virus (EEE)), vesicular stomatitis virus (VSV), fowl pox virus, pseudorabies, herpes simplex viruses, retroviruses, HIV pseudoviruses, and many others. Any vector that transduces genetic material into a cell, and, if replication is desired, which is replicable and viable in the relevant host can be used. Viral and bacterial vectors serving as delivery vehicles can be attenuated; attenuation should be sufficient to decrease if not eliminate induction of undesirable disease symptoms. Additional details regarding suitable expression vectors are provided below.

A vector of the invention comprising a nucleic acid sequence of the invention as described herein (e.g., a recombinant nucleic acid sequence encoding a recombinant gp120 polypeptide variant), as well as an appropriate promoter or control sequence, can be employed to transform an appropriate host to permit the host to express the protein. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells, such as Chinese Hamster Ovary (CHO) (e.g., CHO-K1), COS (e.g., COS-1, COS-7), baby hamster kidney (BHK), and Human Embryonic Kidney (HEK) (e.g., HEK 293), Bowes melanoma cells, and plant cells. It is understood that not all cells or cell lines need to be capable of producing fully functional polypeptides of the invention or fragments thereof; for example, a recombinant gp120 full-length or core polypeptide variant or a WT HIV-1 gp120 polypeptide may be produced in a bacterial, viral, mammalian or other expression system. The invention is not limited by the host cells employed. Additional details regarding suitable host cells are provided below.

In bacterial systems, a number of expression vectors may be selected depending upon the use intended for the desired polypeptide or fragment thereof. For example, when large quantities of a particular polypeptide or fragments thereof are needed for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which nucleotide coding sequence of interest (e.g., nucleotide sequence encoding a recombinant gp120 core or full-length polypeptide variant) may be ligated into the vector in-frame with sequences for the amino-terminal Met and the subsequent 7 residues of beta-galactosidase so that a hybrid protein is produced; pIN vectors (Van Heeke & Schuster (1989) J. Biol. Chem. 264:5503-5509); pET vectors (Novagen, Madison Wis.); and the like.

Similarly, in the yeast Saccharomyces cerevisiae, a number of vectors comprising constitutive or inducible promoters such as alpha factor, alcohol oxidase, and PGH may be used for production of the gp120 polypeptide variants of the invention. For reviews, see Ausubel, supra, Berger, supra, and Grant et al. (1987) Meth. Enzymol. 153:516-544.

In mammalian host cells, a number of expression systems, such as viral-based systems, may be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome results in a viable virus capable of expressing a polypeptide of interest (e.g., gp120 polypeptide variant) in infected host cells (Logan and Shenk (1984) Proc. Natl. Acad. Sci. USA 81:3655-3659). In addition, transcription enhancers, such as the Rous sarcoma virus (RSV) enhancer, are used to increase expression in mammalian host cells.

A vector, e.g., expression vector, or polynucleotide of the invention can comprise one or more expression control sequences. An expression control sequence is typically associated with and/or operably linked to a nucleic acid sequence of the invention, such as a nucleic acid sequence encoding a recombinant gp120 polypeptide variant. An expression control sequence is typically a nucleotide sequence that promotes, enhances, or controls expression (typically transcription) of another nucleotide sequence. Suitable expression control sequences that may be employed include a promoter, including a constitutive promoter, inducible promoter, and/or repressible promoter, an enhancer for amplifying expression, an initiation sequence, a termination translation sequence, a splicing control sequence, and the like.

When a nucleic acid of the invention (e.g., a recombinant nucleic acid encoding a recombinant gp120 core or full-length polypeptide variant) is included in a vector, the nucleic acid is typically operatively linked to an appropriate transcription control sequence (promoter) to direct mRNA synthesis. Promoters exert a particularly important impact on the level of recombinant polypeptide expression. Any suitable promoter can be utilized. Examples of suitable promoters include the cytomegalovirus (CMV) promoter with or without the first intron (intron A), the HIV long terminal repeat promoter, the phosphoglycerate kinase (PGK) promoter, Rous sarcoma virus (RSV) promoters, such as RSV long terminal repeat (LTR) promoters, SV40 promoters, mouse mammary tumor virus (MMTV) promoters, HSV promoters, such as the Lap2 promoter or the herpes thymidine kinase promoter (as described in, e.g., Wagner et al. (1981) Proc. Natl. Acad. Sci. 78:144-145), promoters derived from SV40 or Epstein Barr virus, adeno-associated viral (AAV) promoters, such as the p5 promoter, metallothionein promoters (e.g., the sheep metallothionein promoter or the mouse metallothionein promoter (see, e.g., Palmiter et al. (1983) Science 222:809-814), the human ubiquitin C promoter, E. coli promoters, such as the lac and trp promoters, phage lambda P_{L} promoter, and other promoters known to control expression of genes in prokaryotic or eukaryotic cells (either directly in the cell or in viruses which infect the cell). Promoters that exhibit strong constitutive baseline expression in mammals, particularly humans, such as CMV promoters, such as the CMV immediate-early promoter (described in, e.g., U.S. Pat. Nos. 5,168,062, 5,385,839, 5,688,688, and 5,658,759), and promoters having substantial sequence identity with such CMV promoters, can be employed. Recombinant promoters having novel or enhanced properties, such as those described in Int'l Pat. Appn Publ. No. WO 02/00897, may also be used.

A promoter that is operably linked to a nucleic acid of the invention (e.g., nucleic acid encoding a recombinant gp120 core or full-length polypeptide variant) for expression of the nucleic acid can have any suitable mechanism of action. Thus, the promoter can be, for example, an “inducible” promoter, (e.g., a growth hormone promoter, metallothionein promoter, heat shock protein promoter, E1B promoter, hypoxia induced promoter, radiation inducible promoter, or adenoviral MLP promoter and tripartite leader), an inducible-repressible promoter, a developmental stage-related promoter (e.g., a globin gene promoter), or a tissue specific promoter (e.g., a smooth muscle cell α-actin promoter, myosin light-chain 1A promoter, or vascular endothelial cadherin promoter). Suitable inducible promoters include ecdysone and ecdysone-analog-inducible promoters. Ecdysone-analog-inducible promoters are commercially available, e.g., through Stratagene (La Jolla, Calif.). If desired, a nucleic acid of the invention can be induced by using an inducible on- and off-gene expression system. Examples of such on- and off-gene expression systems include the Tet-On™ Gene Expression System and Tet-Off™ Gene Expression System, respectively (Clontech, Palo Alto, Calif.; see, e.g., Clontech Catalog 2000, pg. 110-111 for a detailed description of each such system). The inducible promoter can be any promoter that is up- and/or downregulated in response to an appropriate signal. Additional inducible promoters include arabinose-inducible promoters, a steroid-inducible promoters (e.g., a glucocorticoid-inducible promoters), as well as pH, stress, and heat-inducible promoters.

The promoter can be, and often is, a host-native promoter, or a promoter derived from a virus that infects a particular host (e.g., a human beta actin promoter, human EF1α promoter, or a promoter derived from a human AAV operably linked to the nucleic acid of interest), particularly where strict avoidance of gene expression silencing due to host immunological reactions to sequences that are not regularly present in the host is of concern. A bi-directional promoter system (as described in, e.g., U.S. Pat. No. 5,017,478) linked to multiple nucleotide sequences of interest can also be utilized.

A vector of the invention can comprise a modified or chimeric promoter sequence and a nucleic acid of interest operably linked to the modified or chimeric promoter sequence. A promoter sequence is “chimeric” if it comprises nucleotides or nucleotide sequences obtained from, derived from, or based upon at least two different sources (e.g., two different regions of an organism's genome, sequences of two different organisms, or an organism's sequence combined with a synthetic sequence). Suitable promoters also include recombinant, mutated, recursively recombined, or shuffled promoters. Minimal promoter elements consisting essentially of a particular TATA-associated sequence can be used alone or in combination with additional promoter elements. TATA-less promoters also can be suitable in some contexts. The promoter and/or other expression control sequences can include one or more regulatory elements have been deleted, modified, or inactivated. Promoters include the promoters described in Int'l Patent Application WO 02/00897, one or more of which can be incorporated into and/or used with nucleic acids and vectors of the invention. Other shuffled and/or recombinant promoters also can be usefully incorporated into and used in the nucleic acids and vectors of the invention to facilitate polypeptide expression.

Other suitable promoters and principles related to the selection, use, and construction of suitable promoters are provided in, e.g., Werner (1999) Mamm Genome 10(2):168-75, Walther et al. (1996) J. Mol. Med. 74(7):379-92, Novina (1996) Trends Genet. 12(9):351-55, Hart (1996) Semin. Oncol. 23(1):154-58, Gralla (1996) Curr. Opin. Genet. Dev. 6(5):526-30, Fassler et al. (1996) Methods Enzymol 273:3-29, Ayoubi et al. (1996), 10(4) FASEB J 10(4):453-60, Goldsteine et al. (1995) Biotechnol. Annu. Rev. 1:105-28, Azizkhan et al. (1993) Crit. Rev. Eukaryot. Gene Expr. 3(4):229-54, Dynan (1989) Cell 58(1):1-4, Levine (1989) Cell 59(3):405-8, and Berk et al. (1986) Annu. Rev. Genet. 20:45-79, as well as U.S. Pat. No. 6,194,191. Other suitable promoters can be identified by use of the Eukaryotic Promoter Database (release 68) (available at the world wide website address epd.isb-sib.ch/) and other similar databases, such as the Transcription Regulatory Regions Database (TRRD) (version 4.1) (available at the world wide website address bionet.nsc.ru/trrd/) and the transcription factor database (TRANSFAC) (available at the world wide website address transfac.gbf.de/TRANSFAC/index.html).

As an alternative to a promoter, particularly in RNA vectors and constructs, a vector or nucleic acid of the invention can comprise one or more internal ribosome entry sites (IRESs), IRES-encoding sequences, or RNA sequence enhancers (Kozak consensus sequence analogs), such as the tobacco mosaic virus omega prime sequence.

A vector or polynucleotide of the invention can include an upstream activator sequence (UAS), such as a Gal4 activator sequence (see, e.g., U.S. Pat. No. 6,133,028) or other suitable upstream regulatory sequence (see, e.g., U.S. Pat. No. 6,204,060).

A vector or polynucleotide of the invention can include a Kozak consensus sequence that is functional in a mammalian cell. The Kozak sequence can be a naturally occurring or modified sequence, such as the modified Kozak consensus sequences described in U.S. Pat. No. 6,107,477.

Specific initiation signals can aid in efficient translation of a coding sequence of the invention, such as a gp120 polypeptide variant-encoding nucleotide sequence. Such signals can be included in a vector of the invention. These signals can include, e.g., the ATG initiation codon and adjacent sequences. In cases where a coding sequence, its initiation codon, and upstream sequences are inserted into an appropriate expression vector, no additional translational control signals may be needed. However, in cases where only a coding sequence (e.g., a mature protein coding sequence), or a portion thereof is inserted, exogenous nucleic acid transcriptional control signals including the ATG initiation codon must be provided. Furthermore, the initiation codon must be in the correct reading frame to ensure transcription of the entire insert. Exogenous transcriptional elements and initiation codons can be of various origins—both natural and synthetic. The efficiency of expression can be enhanced by the inclusion of enhancers appropriate to the cell system in use (see, e.g., Scharf et al., Results Probl. Cell. Differ. 20:125-62 (1994); and Bittner et al., Meth. Enzymol. 153:516-544 (1987)). Suitable enhancers include the Rous sarcoma virus (RSV) enhancer and the RTE enhancers described in U.S. Pat. No. 6,225,082.

The skilled artisan will recognize that the introduction of a start codon (ATG) to the 5′ end of a particular nucleotide sequence of interest usually results in the addition of an N-terminal methionine to the encoded amino acid sequence when the sequence is expressed in a mammalian cell (other modifications may occur in bacterial and/or other eukaryotic cells, such as introduction of an formyl-methionine residue at a start codon). For expression of a nucleic acid of the invention in eukaryotic cells, a start codon and a nucleotide sequence encoding a signal peptide are typically be included at the 5′ end of a nucleic acid sequence of the invention (e.g., SEQ ID NO:23), and a termination codon is typically included at the C terminus of the nucleic acid (e.g., SEQ ID NO:23). An exemplary signal peptide sequence is the tissue plasminogen activator signal peptide sequence (SEQ ID NO:52); the nucleic acid sequence encoding the tissue plasminogen activator signal peptide is shown in SEQ ID NO:53. Termination sequences are discussed in detail below.

Such elements can be included in the vector construct of choice. Upon expression, the polypeptide variant encoded by the nucleic acid (e.g., SEQ ID NO:23) will initially include an N-terminal methionine residue and the signal peptide sequence. However, the N-terminal methionine and signal peptide sequence will be cleaved upon secretion, thereby generating the encoded polypeptide (e.g., SEQ ID NO:1).

The expression level of a nucleic acid of the invention (or a corresponding polypeptide of the invention (e.g., recombinant gp120 polypeptide variant) for comparative purposes) can be assessed by any suitable technique. Examples of such techniques include Northern Blot analysis (discussed in, e.g., McMaster et al., Proc. Natl. Acad. Sci. USA 74(11):4835-38 (1977) and Sambrook, infra), reverse transcriptase-polymerase chain reaction (RT-PCR) (as described in, e.g., U.S. Pat. No. 5,601,820 and Zaheer et al., Neurochem. Res. 20:1457-63 (1995)), and in situ hybridization techniques (as described in, e.g., U.S. Pat. Nos. 5,750,340 and 5,506,098). Quantification of proteins also can be accomplished by the Lowry assay and other classification protein quantification assays (see, e.g., Bradford, Anal. Biochem. 72:248-254 (1976) and Lowry et al., J. Biol. Chem. 193:265 (1951)). Western blot analysis of recombinant polypeptides of the invention obtained from the lysate of cells transfected with polynucleotides encoding such recombinant polypeptides is another suitable technique for assessing levels of recombinant polypeptide expression.

A vector, e.g., expression vector, or polynucleotide of the invention can comprise a ribosome-binding site for translation initiation and a transcription-terminating region. A suitable transcription-terminating region is, for example, a polyadenylation sequence that facilitates cleavage and polyadenylation of an RNA transcript produced from a DNA sequence. Any suitable polyadenylation sequence can be used, including a synthetic optimized sequence, as well as the polyadenylation sequence of BGH (Bovine Growth Hormone), human growth hormone gene, polyoma virus, TK (Thymidine Kinase), EBV (Epstein Barr Virus), rabbit beta globin, and the papillomaviruses, including human papillomaviruses and BPV (Bovine Papilloma Virus). Suitable polyadenylation (polyA) sequences also include the SV40 (human Sarcoma Virus-40) polyadenylation sequence and the BGH polyA sequence. Such polyA sequences are described in, e.g., Goodwin et al. (1998) Nucleic Acids Res. 26(12):2891-8, Schek et al. (1992) Mol. Cell. Biol. 12(12):5386-93, and van den Hoff et al. (1993) Nucleic Acids Res. 21(21):4987-8. Additional principles related to selection of appropriate polyadenylation sequences are described in, e.g., Levitt et al. (1989) Genes Dev. 1989 3(7):1019-1025, Jacob et al. (1990) Crit. Rev. Eukaryot. Gene Expr. 1(1):49-59, Chen et al. (1995) Nucleic Acids Res. 23(14):2614-2620, Moreira et al. (1995) EMBO J. 14(15):3809-3819, Carswell et al. (1989) Mol. Cell. Biol. 1989 9(10):4248-4258.

A vector or polynucleotide of the invention can further comprise site-specific recombination sites, which can be used to modulate transcription of a nucleotide sequence of interest, as described in, e.g., U.S. Pat. Nos. 4,959,317, 5,801,030 and 6,063,627, European Patent Application No. 0 987 326 and Int'l Patent Application Publ. No. WO 97/09439.

In one aspect, a vector or polynucleotide of the invention comprises a T7 RNA polymerase promoter operably linked to a nucleic acid sequence of interest, facilitating the synthesis of single-stranded RNAs from the nucleic acid sequence. T7 and T7-derived sequences are known, as are expression systems using T7 (see, e.g., Tabor and Richardson (1986) Proc. Natl. Acad. Sci. USA 82:1074, Studier and Moffat (1986) J. Mol. Biol. 189:113, and Davanloo et al. (1964) Proc. Natl. Acad. Sci. USA 81:2035). Nucleic acids comprising a T7 RNA polymerase and a nucleotide sequence encoding at least one recombinant polypeptide of the invention are provided.

A vector or polynucleotide of the invention can also comprise a nucleic acid encoding a secretion/localization sequence, to target polypeptide expression to a desired cellular compartment, membrane, or organelle, or to direct polypeptide secretion to the periplasmic space or into the cell culture media. Such sequences are known in the art, and include secretion leader peptides or signal peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like. Polynucleotides of the invention can be fused, for example, in-frame to such a nucleic acid encoding a secretion and/or localization sequence. Polypeptides expressed by such polynucleotides of the invention may include the amino acid sequence corresponding to the secretion and/or localization sequence(s).

In addition, a vector or polynucleotide of the invention can comprise one or more selectable marker genes to provide a phenotypic trait for selection of transformed host cells, such as dihydrofolate reductase resistance, neomycin resistance, G418 resistance, puromycin resistance, and/or blasticidin resistance for eukaryotic cell culture, or such as tetracycline or ampicillin resistance in E. coli.

A vector or polynucleotide of the invention can also comprise an origin of replication useful for propagation in a microorganism. The bacterial origin of replication (Ori) utilized is preferably one that does not adversely affect gene expression in mammalian cells. Examples of useful origin of replication sequences include the f1 phage ori, RK2 oriV, pUC ori, and the pSC101 ori. Origin of replication sequences include the CoIEI on and the p15 (available from plasmid pACYC177, New England Biolab, Inc.), alternatively another low copy on sequence (similar to p15) can be desirable in some contexts. The nucleic acid in this respect desirably acts as a shuttle vector, able to replicate and/or be expressed in both eukaryotic and prokaryotic hosts (e.g., a vector comprising an origin of replication sequences recognized in both eukaryotes and prokaryotes).

The invention includes a naked DNA or RNA vector, including, for example, a linear expression element (as described in, e.g., Sykes and Johnston (1997) Nat Biotech 17:355-59), a compacted nucleic acid vector (as described in, e.g., U.S. Pat. No. 6,077,835 and/or Int'l Patent Appn WO 00/70087), a plasmid vector such as pBR322, pUC 19/18, or pUC118/119, a “midge” minimal-sized nucleic acid vector (as described in, e.g., Schakowski et al. (2001) Mol. Ther. 3:793-800) or as a precipitated nucleic acid vector construct, such as a CaPO₄ precipitated construct (as described in, e.g., Int'l Patent Appn WO 00/46147, Benvenisty and Reshef (1986) Proc. Natl. Acad. Sci. USA 83:9551-55, Wigler et al. (1978), Cell 14:725, and Coraro and Pearson (1981) Somatic Cell Genetics 7:603), comprising a nucleic acid of the invention. For example, the invention provides a naked DNA plasmid comprising SEQ ID NO:37 operably linked to a CMV promoter or CMV promoter variant and a suitable polyadenylation sequence. Naked nucleotide vectors and the usage thereof are known in the art (see, e.g., U.S. Pat. Nos. 5,589,466 and 5,973,972).

A vector of the invention typically is an expression vector that is suitable for expression in a bacterial system, mammalian system, or other system (as opposed to a vector designed for replicating the nucleic acid sequence without expression, which can be referred to as a cloning vector). For example, in one aspect, the invention provides a bacterial expression vector comprising a nucleic acid sequence of the invention (e.g., recombinant gp120 polypeptide variant-encoding nucleic acid sequence). Suitable vectors include, for example, vectors which direct high level expression of fusion proteins that are readily purified (e.g., multifunctional E. coli cloning and expression vectors such as BLUESCRIPT (Stratagene), pIN vectors (Van Heeke & Schuster, J. Biol. Chem. 264:5503-5509 (1989); pET vectors (Novagen, Madison Wis.); and the like). While such bacterial expression vectors can be useful in expressing particular polypeptides of the invention, glycoproteins of the invention are preferably expressed in eukaryotic cells and as such the invention also provides eukaryotic expression vectors.

The expression vector can be a vector suitable for expression of the nucleic acid of the invention in a yeast cell. Any vector suitable for expression in a yeast system can be employed. Suitable vectors for use in, e.g., Saccharomyces cerevisiae include, for example, vectors comprising constitutive or inducible promoters such as alpha factor, alcohol oxidase and PGH (reviewed in: Ausubel, supra, Berger, supra, and Grant et al., Meth. Enzymol. 153:516-544 (1987)).

Usually, the expression vector will be a vector suitable for expression of a nucleic acid of the invention (e.g., gp120 polypeptide variant-encoding nucleic acid sequence) in an animal cell, such as an insect cell (e.g., a SF-9 cell) or a mammalian cell (e.g., a CHO cell, 293 cell, HeLa cell, human fibroblast cell, or similar well-characterized cell). Suitable mammalian expression vectors are known in the art (see, e.g., Kaufman, Mol. Biotechnol. 16(2):151-160 (2000), Van Craenenbroeck, Eur. J. Biochem. 267(18):5665-5678 (2000), Makrides, Protein Expr. Purif. 17(2):183-202 (1999), and Yarranton, Curr. Opin. Biotechnol. 3(5):506-511 (1992)). Suitable insect cell plasmid expression vectors also are known (see, e.g., Braun, Biotechniques 26(6):1038-1040, 1042 (1999)).

An expression vector typically can be propagated in a host cell, which may be a eukaryotic cell (such as a mammalian cell, yeast cell, or plant cell) or a prokaryotic cell, such as a bacterial cell. Introduction of a nucleic acid vector or expression vector into the host cell (e.g., transfection) can be effected by calcium phosphate transfection (see, e.g., calcium phosphate co-precipitation method of Graham et al., Virology 52:456-457 (1973)), DEAE-Dextran mediated transfection, electroporation, gene or vaccine gun, injection, lipofection and biolistics or other common techniques (see, e.g., Kriegler, GENE TRANSFER AND EXPRESSION: A LABORATORY MANUAL, Stockton Press (1990); see Davis, L., Dibner, M., and Battey, I., BASIC METHODS IN MOLECULAR BIOLOGY (1986) for a description of in vivo, ex vivo, and in vitro methods). Cells comprising these and other vectors of the invention form an important part of the invention.

Additional nucleic acids provided by the invention include cosmids. Any suitable cosmid vector can be used to replicate, transfer, and express the nucleic acid sequence of the invention. Typically, a cosmid comprises a bacterial oriV, an antibiotic selection marker, a cloning site, and either one or two cos sites derived from bacteriophage lambda. The cosmid can be a shuttle cosmid or mammalian cosmid, comprising a SV40 oriV and, desirably, suitable mammalian selection marker(s). Cosmid vectors are further described in, e.g., Hohn et al. (1988) Biotechnology 10:113-27.

Nucleic acids of the invention can be included in and/or administered to a host or host cell in the form of a suitable delivery vehicle (i.e., a vector). The vector can be any suitable vector, including chromosomal, non-chromosomal, and synthetic nucleic acid vectors, or other vectors described above, and may include any combination of the above-described expression elements and/or other transfection-facilitating and/or expression-promoting sequence elements. Examples of such vectors include viruses, bacterial plasmids, phages, cosmids, phagemids, derivatives of SV40, baculovirus, yeast plasmids, vectors derived from combinations of plasmids and phage DNA, and viral nucleic acid (RNA or DNA) vectors, polylysine, and bacterial cells.

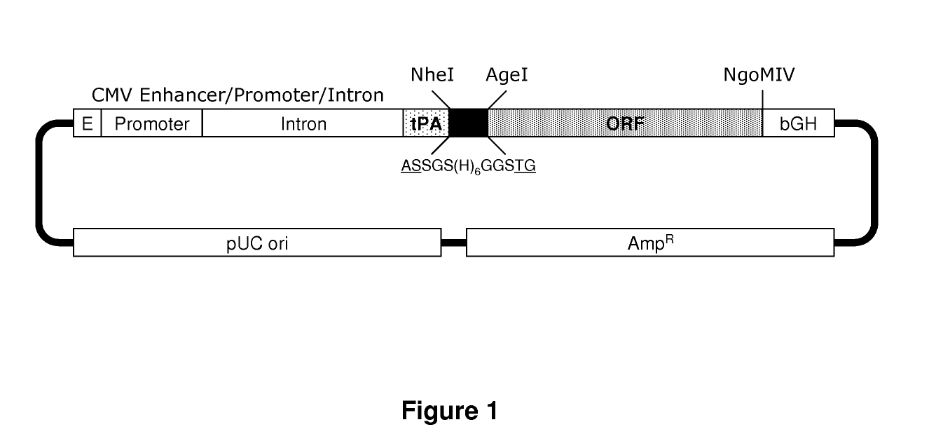

Delivery of a recombinant DNA sequence of the invention can be accomplished with a naked DNA plasmid or plasmid associated with one or more transfection-enhancing agents, as discussed further herein. The plasmid DNA vector can have any suitable combination of features. Plasmid DNA vectors may comprise a strong promoter/enhancer region (e.g., human CMV, RSV, SV40, SL3-3, MMTV, or HIV LTR promoter), an effective poly(A) termination sequence, an origin of replication for plasmid product in E. coli, an antibiotic resistance gene as selectable marker, and a convenient cloning site (e.g., a polylinker). A particular plasmid vector for delivery of the nucleic acid of the invention in this respect is the vector pMAmp, the construction and features of which are described in the Examples and FIG. 1. The plasmid vector typically includes Optionally, such a plasmid vector includes at least one immunostimulatory sequence (ISS) and/or at least one gene encoding a suitable cytokine adjuvant (e.g., a GM-CSF sequence, IL-2 sequence, or both), as further described elsewhere herein.

In another aspect, the invention provides a non-nucleic acid vector comprising at least one nucleic acid or polypeptide of the invention. Such a non-nucleic acid vector includes, e.g., but is not limited to, a recombinant virus, a viral nucleic acid-protein conjugate (which, with recombinant viral particles, may sometimes be referred to as a viral vector), or a cell, such as recombinant (and usually attenuated) Salmonella, Shigella, Listeria, and Bacillus Calmette-Guérin (BCG) bacterial cells. Thus, for example, the invention provides a viral vector or bacterial vector comprising a nucleic acid of the sequence of the invention. Any suitable viral or bacterial vector can be used in this respect and a number are known in the art. A viral vector can comprise any number of viral polynucleotides, alone (a viral nucleic acid vector) or more commonly in combination with one or more (typically two, three, or more) viral proteins, which facilitate delivery, replication, and/or expression of the nucleic acid of the invention in a desired host cell.

In one aspect, intracellular bacteria (e.g., Listeria monocytogenes) can be used to deliver a nucleic acid of the invention. An exemplary bacterial vector for plasmid DNA delivery of one or more nucleic acids of the invention is Listeria monocytogenes (Lieberman et al., Vaccine 20:2007-2010 (2002)). For example, such a bacterium can be engineered to include at least one nucleic acid sequence of the invention that induces an HIV immune response. Such an HIV vaccine vector comprising a nucleic acid of the invention is believed capable of inducing a detectable CTL response, a CD4 helper response, and/or an antibody response.

The invention includes recombinant or isolated viral vectors that have been modified to comprise one or more nucleic acids or polypeptides of the invention. A viral vector may include a polynucleotide comprising all or part of a viral genome, a viral protein/nucleic acid conjugate, a virus-like particle (VLP), a vector similar to those described in U.S. Pat. No. 5,849,586 and Int'l Patent Appn WO 97/04748, or an intact virus particle comprising one or more viral nucleic acids, and the viral vector is typically engineered to include at least one nucleic acid and/or polypeptide of the invention. A viral vector (i.e., a recombinant virus) can comprise a wild-type viral particle or a modified viral particle, particular examples of which are discussed below. Numerous viruses are typically used as vectors for the delivery of exogenous nucleic acids, including at least one nucleic acid of the invention, such as a nucleic acid encoding a gp120 core or full-length polypeptide variant described herein. Such vectors include recombinantly modified enveloped or non-enveloped DNA and RNA viruses, typically selected from baculoviridiae, parvoviridiae, picomoviridiae, herpesveridiae, poxyiridae, adenoviridiae, or picornnaviridiae. Viral vectors may be wild-type or may be modified by recombinant nucleic acid techniques to be replication deficient, replication competent, or conditionally replicating.

The viral vector can be a vector that requires the presence of another vector or wild-type virus for replication and/or expression (i.e., a helper-dependent virus), such as an adenoviral vector amplicon. Typically, such viral vectors consist essentially of a wild-type viral particle, or a viral particle modified in its protein and/or nucleic acid content to increase transgene capacity or aid in transfection and/or expression of the nucleic acid (examples of such vectors include the herpes virus/AAV amplicons). The viral genome may be modified to include inducible promoters that achieve replication or expression only under certain conditions.

The viral vector can be derived from or comprise a virus that normally infects animals, preferably vertebrates, such as mammals, including, e.g., humans. Suitable viral vector particles in this respect, include, for example, adenoviral vector particles (including any virus of or derived from a virus of the adenoviridae), adeno-associated viral vector particles (AAV vector particles) or other parvoviruses and parvoviral vector particles, papillomaviral vector particles, Semliki-Forest viral vector, flaviviral vectors, picornaviral vectors, alphaviral vectors, herpes viral vectors, pox virus vectors, retroviral vectors, including lentiviral vectors. Examples of such viruses and viral vectors are provided in, e.g., Fields Virology, supra, Fields et al., eds., VIROLOGY, Raven Press, Ltd., New York (3^{rd} ed., 1996 and 4^{th} ed., 2001), ENCYCLOPEDIA OF VIROLOGY, R. G. Webster et al., eds., Academic Press (2^{nd} ed., 1999), FUNDAMENTAL VIROLOGY, Fields et al., eds., Lippincott-Raven (3^{rd} ed., 1995), Levine, “Viruses,” Scientific American Library No. 37 (1992), MEDICAL VIROLOGY, D. O. White et al., eds., Academic Press (2^{nd} ed. 1994), and INTRODUCTION TO MODERN VIROLOGY, Dimock, N. J. et al., eds., Blackwell Scientific Publications, Ltd. (1994).

Viral vectors that can be employed with nucleic acids of the invention and the methods described herein include adeno-associated virus vectors, which are reviewed in, e.g., Carter (1992) Curr. Opinion Biotech. 3:533-539 (1992) and Muzcyzka (1992) Curr. Top. Microbiol. Immunol. 158:97-129 (1992). Additional types and aspects of AAV vectors are described in, e.g., Buschacher et al., Blood 5(8):2499-504, Carter, Contrib. Microbiol. 4:85-86 (2000), Smith-Arica, Curr. Cardiol. Rep. 3(1):41-49 (2001), Taj, J. Biomed. Sci. 7(4):279-91 (2000), Vigna et al., J. Gene Med. 2(5):308-16 (2000), Klimatcheva et al., Front. Biosci. 4:D481-96 (1999), Lever et al., Biochem. Soc. Trans. 27(6):841-47 (1999), Snyder, J. Gene Med. 1(3):166-75 (1999), Gerich et al., Knee Surg. Sports Traumatol. Arthrosc. 5(2):118-23 (1998), and During, Adv. Drug Deliv. Review 27(1):83-94 (1997), and U.S. Pat. Nos. 4,797,368, 5,139,941, 5,173,414, 5,614,404, 5,658,785, 5,858,775, and 5,994,136, as well as other references discussed elsewhere herein). Adeno-associated viral vectors can be constructed and/or purified using the methods set forth, for example, in U.S. Pat. No. 4,797,368 and Laughlin et al., Gene 23:65-73 (1983).

Another type of viral vector that can be employed with nucleic acids and methods of the invention is a papillomaviral vector. Suitable papillomaviral vectors are known in the art and described in, e.g., Hewson (1999) Mol. Med. Today 5(1):8, Stephens (1987) Biochem J 248(1):1-11, and U.S. Pat. No. 5,719,054. Exemplary papillomaviral vectors are provided in WO 99/21979.

Another viral vector that can be used with nucleic acids of the invention is the Coxsackie virus (see, e.g., Halim et al., AIDS Research and Human Retroviruses 16(15):1551-1558 (2000)). Such a vector comprising a nucleic acid of the invention is believed able to induce CTL and CD4+ helper T cell responses, such as HIV-specific T cell responses, and/or anti-HIV antibody responses. Some such vectors comprising nucleic acids of the invention are believed capable of inducing T cell immunity against HIV.

Alphavirus vectors can be gene delivery vectors in other contexts. Alphavirus vectors are known in the art and described in, e.g., Carter (1992) Curr Opinion Biotech 3:533-539, Muzcyzka (1992) Curr. Top. Microbiol. Immunol. 158:97-129, Schlesinger Expert Opin. Biol. Ther. (2001) 1(2):177-91, Polo et al., Dev. Biol. (Base1). 2000; 104:181-5, Wahlfors et al., Gene Ther. (2000) 7(6):472-80, Colombage et al., Virology. (1998) 250(1):151-63, and Int'l Patent Appn Publ. Nos. WO 01/81609, WO 00/39318, WO 01/81553, WO 95/07994, and WO 92/10578.

Another advantageous group of viral vectors are the herpes viral vectors. Examples of herpes viral vectors are described in, e.g., Lachmann et al., Curr. Opin. Mol. Ther. (1999) 1(5):622-32, Fraefel et al., Adv. Virus Res. (2000) 55:425-51, Huard et al., Neuromuscul. Disord. (1997) 7(5):299-313, Glorioso et al., Annu. Rev. Microbiol. (1995) 49:675-710, Latchman, Mol. Biotechnol. (1994) 2(2):179-95, and Frenkel et al., Gene Ther. (1994) Suppl 1:S40-6, as well as U.S. Pat. Nos. 6,261,552 and 5,599,691.

Retroviral vectors, including lentiviral vectors, also can be advantageous gene delivery vehicles in particular contexts. There are numerous retroviral vectors known in the art. Examples of retroviral vectors are described in, e.g., Miller, Curr Top Microbiol. Immunol. (1992) 158:1-24; Salmons and Gunzburg (1993) Human Gene Ther. 4:129-141; Miller et al. (1994) Meth. Enzymol. 217:581-599, Weber et al., Curr. Opin. Mol. Ther. (2001) 3(5):439-53, Hu et al., Pharmacol. Rev. (2000) 52(4):493-511, Kim et al., Adv. Virus Res. (2000) 55:545-63, Palu et al., Rev. Med. Virol. (2000) 10(3):185-202, and Takeuchi et al., Adv. Exp. Med. Biol. (2000) 465:23-35, as well as U.S. Pat. Nos. 6,326,195, 5,888,502, 5,580,766, and 5,672,510.

Baculovirus vectors are another advantageous group of viral vectors, particularly for the production of polypeptides of the invention. The production and use of baculovirus vectors is known (see, e.g., Kost, Curr. Opin. Biotechnol. 10(5):428-433 (1999) and Jones, Curr. Opin. Biotechnol. 7(5):512-516 (1996)). Where the vector is used for therapeutic uses (e.g., to induce an immune response against HIV) the vector will be selected such that it is able to adequately infect (or in the case of nucleic acid vectors transfect or transform) target cells in which the desired therapeutic effect is desired.

Adenoviral vectors also can be suitable viral vectors for gene transfer. Adenoviral vectors are well known in the art and described in, e.g., Graham et al. (1995) Mol. Biotechnol. 33(3):207-220, Stephenson (1998) Clin. Diagn. Virol. 10(2-3):187-94, Jacobs (1993) Clin Sci (Lond). 85(2):117-22, U.S. Pat. Nos. 5,922,576, 5,965,358 and 6,168,941 and International Patent Applications WO 98/22588, WO 98/56937, WO 99/15686, WO 99/54441, and WO 00/32754. Adenoviral vectors, herpes viral vectors, and Sindbis viral vectors, useful in the practice of the invention and suitable for organismal in vivo transduction and expression of nucleic acids of the invention, are generally described in, e.g., Jolly (1994) Cancer Gene Therapy 1:51-64, Latchman (1994) Molec. Biotechnol. 2:179-195, and Johanning et al. (1995) Nucl. Acids Res. 23:1495-1501.

Other suitable viral vectors for transduction and expression include pox viral vectors. Examples of such vectors are discussed in, e.g., Berencsi et al., J. Infect. Dis. (2001) 183(8):1171-9; Rosenwirth et al., Vaccine (2001)19(13-14):1661-70; Kittlesen et al., J. Immunol. (2000) 164(8):4204-11; Brown et al., Gene Ther. (2000) 7(19):1680-9; Kanesa-thasan et al., Vaccine (2000) 19(4-5):483-91; Sten (2000) Drug 60(2):249-71. Vaccinia virus vectors (e.g., Modified Vaccinia Ankara (MVA) vectors and MVA-derived vectors) are particularly advantageous pox virus vectors in some contexts, as are fowl pox virus vectors, canary pox virus vectors, and other avipox virus vectors. Examples of such vaccinia virus vectors and uses thereof are provided in, e.g., Venugopal et al. (1994) Res. Vet. Sci. 57(2):188-193, Moss (1994) Dev. Biol. Stand. 82:55-63 (1994), Weisz et al. (1994) Mol. Cell. Biol. 43:137-159, Mahr and Payne (1992) Immunobiology 184(2-3):126-146, Hruby (1990) Clin. Microbiol. Rev. 3(2):153-170, and Int'l Patent Appn Publ. Nos. WO 92/07944, WO 98/13500, and WO 89/08716. Related canary pox, avipox, and fowl pox viruses also are known in the art (see, e.g., Ratliff et al., Acta Urol Belg. (1996) 64(2):85 and Paoletti, Proc. Natl. Acad. Sci. USA (1996) 93(21):11349-53).

The virus vector may be replication-deficient in a host cell. Adeno-associated virus (AAV) vectors, which are naturally replication-deficient in the absence of complementing adenoviruses or at least adenovirus gene products (provided by, e.g., a helper virus, plasmid, or complementation cell), are included. By “replication-deficient” is meant that the viral vector comprises a genome that lacks at least one replication-essential gene function. A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient viral vector. The essential gene functions of the viral vector particle vary with the type of viral vector particle at issue. Examples of replication-deficient viral vector particles are described in, e.g., Marconi et al., Proc. Natl. Acad. Sci. USA 93(21):11319-20 (1996), Johnson and Friedmann, Methods Cell Biol. 43 (pt. A):211-30 (1994), Timiryasova et al., J. Gene Med. 3(5):468-77 (2001), Burton et al., Stem Cells 19(5):358-77 (2001), Kim et al., Virology 282(1):154-67 (2001), Jones et al., Virology 278(1):137-50 (2000), Gill et al., J. Med. Virol. 62(2):127-39 (2000), Chen and Engleman, J. Virol. 74(17):8188-93 (2000), Marconi et al., Gene Ther. 6(5):904-12 (1999), Krisky et al., Gene Ther. 5(11):1517-30 (1998), Bieniasz et al., Virology 235(1):65-72 (1997), Strayer et al., Biotechniques 22(3):447-50 (1997), Wyatt et al., Vaccine 14(15):1451-8 (1996), and Penciolelli et al., J. Virol. 61(2):579-83 (1987). Other replication-deficient vectors are based on simple MLV vectors. See, e.g., Miller et al. (1990) Mol Cell Biol 10:4239 (1990); Kolberg (1992) J. NIH Res. 4:43, and Cornetta et al. (1991) Hum. Gene. Ther. 2:215). Canary pox vectors are advantageous in infecting human cells but being naturally incapable of replication therein (i.e., without genetic modification).

The basic construction of recombinant viral vectors is well understood in the art and involves using standard molecular biological techniques such as those described in, e.g., Sambrook et al., MOLECULAR CLONING: A LABORATORY MANUAL (Cold Spring Harbor Press 1989) and the third edition thereof (2001), Ausubel et al., CURRENT PROTOCOLS IN MOLECULAR BIOLOGY (Wiley Interscience Publishers 1995), and Watson, supra, and several of the other references mentioned herein. For example, adenoviral vectors can be constructed and/or purified using the methods set forth, for example, in Graham et al., Mol. Biotechnol. 33(3):207-220 (1995), U.S. Pat. No. 5,965,358, Donthine et al., Gene Ther. 7(20):1707-14 (2000), and other references described herein. Adeno-associated viral vectors can be constructed and/or purified using the methods set forth, for example, in U.S. Pat. No. 4,797,368 and Laughlin et al., Gene 23:65-73 (1983). Similar techniques are known in the art with respect to other viral vectors, particularly with respect to herpes viral vectors (see e.g., Lachman et al., Curr. Opin. Mol. Ther. 1(5):622-32 (1999)), lentiviral vectors, and other retroviral vectors. In general, the viral vector comprises an insertion of the nucleic acid (for example, a wild-type adenoviral vector can comprise an insertion of up to 3 KB without deletion), or, more typically, comprises one or more deletions of the virus genome to accommodate insertion of the nucleic acid and additional nucleic acids, as desired, and to prevent replication in host cells.

In one aspect, the viral vector is a targeted viral vector, comprising a restricted or expanded tropism as compared to a wild-type viral particle of similar type. Targeting is typically accomplished by modification of capsid and/or envelope proteins of the virus particle. Examples of targeted virus vectors and related principles are described in, e.g., International Patent Applications WO 92/06180, WO 94/10323, WO 97/38723, and WO 01/28569, and WO 00/11201, Engelstadter et al., Gene Ther., 8(15), 1202-6 (2001), van Beusechem et al., Gene Ther. 7(22):1940-6 (2000), Boerger et al., Proc. Natl. Acad. Sci. USA 96(17):9867-72 (1999), Bartlett et al., Nat. Biotechnol. 17(2):181-6 (1999), Girod et al., Nat. Med. 5(9):1052-56 (as modified by the erratum in Nat. Med. 5(12):1438) (1999), J. Gene Med. (1999) 1(5):300-11, Karavanas et al., Crit. Rev. Oncol. Hematol. (1998) 28(1):7-30, Wickham et al., J. Virol. 71(10):7663-9 (1997), Cripe et al., Cancer Res. 61(7):2953-60 (2001), van Deutekom et al., J. Gene Med. 1(6):393-9 (1999), McDonald et al., J. Gene Med. 1(2):103-10 (1999), Peng, Curr. Opin. Biotechnol. (1999) 10(5):454-7, Staba et al., Cancer Gene Ther. 7(1):13-9 (2000), Kibbe et al., Arch. Surg. 135(2):191-7 (2000), Harari et al., Gene Ther. 6(5):801-7 (2000), and Bouri et al., Hum Gene Ther. 10(10):1633-40 (1999), and Laquerre et al., J. Virol. 72(12):9683-97 (1997), Buchholz, Curr. Opin. Mol. Ther. (1999) 1(5):613-21, U.S. Pat. Nos. 6,261,554, 5,962,274, 5,695,991, and 6,251,654, and European Patent Application Nos. 1 002 119 and 1 038 967. Particular targeted vectors and techniques for producing such vectors are provided in International Patent Application WO 99/23107.

A viral vector particle comprising a nucleic acid of the invention can be a chimeric viral vector particle (i.e., a virus encoded by the combination of two or more viral genomes). Examples of chimeric viral vector particles are described in, e.g., Reynolds et al., Mol. Med. Today 5(1):25-31 (1999), Boursnell et al., Gene 13:311-317 (1991), Dobbe et al., Virology 288(2): 283-94 (2001), Grene et al., AIDS Res. Human Retroviruses 13(1), 41-51 (1997), Reimann et al., J. Virol. 70(10):6922-8 (1996), Li et al., J. Virol. 67(11):6659-66 (1993), Dong et al., J. Virol. 66(12):7374-82 (1992), Wahlfors, Hum. Gene Ther. (1999) 10(7):1197-206, Reynolds et al., Mol. Med. Today 5(1):25-31 (1999), Boursnell et al., Gene 13:311-317 (1991). and U.S. Pat. Nos. 5,877,011, 6,183,753, 6,146,643, and 6,025,341.

Non-viral vectors, such as, e.g., DNA plasmids, naked nucleic acids, and nucleic acid complexed with a delivery vehicle such as a liposome, also can be associated with molecules that target the vector to a particular region in the host (e.g., a particular organ, tissue, and/or cell type). For example, a nucleotide can be conjugated to a targeting protein, such as a viral protein that binds a receptor or a protein that binds a receptor of a particular target (e.g., by a modification of the techniques provided in Wu and Wu, J. Biol. Chem. 263(29):14621-24 (1988)). Targeted cationic lipid compositions also are known in the art (see, e.g., U.S. Pat. No. 6,120,799). Other techniques for targeting genetic constructs are provided in Int'l Patent Application Publ. No. WO 99/41402.

Virus-Like Particles

HIV viral proteins are known to form viral-like or virus-like particles (VLPs). See, e.g., Kang, C. Y. et al., Biol. Chem. 380(3):353-64 (1999); Akahata, W. et al., J. Virol. 79(1):626-61 (2005); Doan, L. X. et al., Rev. Med. Virol. 15(2):75-88 (2005); and U.S. Pat. No. 6,099,847. A VLP lacks the viral components that are required for virus replication and thus represents a non-replicating, non-infectious particle. Because VLPs neither replicate nor contain the HIV genome, they offer advantages as vaccines over live-attenuated and whole-inactivated vaccines—both of which pose safety concerns for human use. VLPs have been shown to be safe for administration to animals and human subjects and to induce potent cellular and humoral immune responses in human and animal subjects to whom they are administered. Doan et al., Kang et al., and Akahata et al., all supra. Furthermore, HIV-1 VLPs are believed to have potential use as HIV vaccines. Crooks et al., Virology 366(2):245-62 (2007). For example, chimeric HIV-1 VLPs constructed using either an HIV or SIV capsid protein and HIV immune-stimulating epitopes have been found to enhance immune stimulation. Doan et al., supra. Chimeric VLPs encoded by a recombinant chimeric gene comprising a nucleotide sequence encoding a modified HIV-2 gag pr45 precursor protein and a nucleotide sequence encoding a modified V3 region of the HIV-1 gp120 protein were found to induce neutralizing antibodies in rabbits immunized with these gag-env VLPs. Kang et al., supra. A DNA plasmid expression vector encoding both an HIV-1 gag protein and an HIV-1 envelope protein was shown to produce an HIV-1 VLP, and vaccination of mice with such plasmids was induced cellular and humoral immune responses. Akahata et al., supra. For a review of VLPs as HIV-1 vaccines, see Doan et al., supra.

The invention broadly encompasses a variety of isolated or recombinant chimeric VLPs comprising at least one gp120 core or full-length polypeptide variant of the invention and/or at least one nucleic acid encoding such variant of the invention. Exemplary VLPs of the invention are presented below. However, the invention is not limited to these specific embodiments. VLPs of the invention are believed capable of inducing a detectable humoral and/or cellular immune response(s) against at least one HIV virus (e.g., at least one HIV-1 virus) upon administration to a subject and thus are believed useful in the prophylactic and therapeutic treatment methods for inducing immune responses against HIV viruses described below. VLPs of the invention are believed to be useful as HIV vaccines and may be used in combination with other HIV vaccine or pharmaceutical approaches to prevent or treat disease associated with HIV infection. VLPs of the invention thus serve as convenient vehicles for delivery or administration to a subject of one or more polypeptides and/or nucleic acids of the invention. Exemplary methods for delivering and administration are described in detail below. See also Doan, L. X. et al., supra; U.S. Pat. No. 5,849,586.

In one aspect, the invention provides a recombinant VLP comprising (1) at least one recombinant polypeptide comprising a sequence having at least 85, 90, 91, 92, 93, 94, 95, 96, 97, 98, 99, or 100% sequence identity to a polypeptide sequence selected from any of SEQ ID NO:1-21 and 56-63 and/or (2) at least one recombinant nucleic acid encoding at least one polypeptide comprising a sequence having at least 85, 90, 91, 92, 93, 94, 95, 96, 97, 98, 99, or 100% sequence identity to a polypeptide sequence selected from any of SEQ ID NO:1-21 and 56-63, or a complementary nucleotide sequence thereof, wherein the VLP induces an immune response in a subject to whom an effective amount is administered. The immune response may comprise a T-cell response or humoral response. The humoral response may comprise the production of neutralizing antibodies against one or more HIV (e.g., HIV-1) viruses or pseudoviruses of the same subtype or of different subtypes or any combination thereof.

A gp120 polypeptide variant of the invention, by itself, is typically not sufficient to form a VLP; for proper formation of a VLP, additional viral core and/or envelope protein components (e.g., HIV gp41) are typically needed. Such components may be obtained or derived from a known HIV virus, such as an HIV-1 virus, and/or from a known non-HIV virus, such as, e.g., a hepatitis B virus (HBV), HBV surface antigen, influenza virus, vesicular stomatitis virus, human papilloma virus (HPV), Foamy virus (Spuma virus), etc. Doan et al., supra. The self-assembling core and/or envelope structure(s) of a number of known viruses can be adapted using recombinant techniques to display, associate with, or comprise one of more of the gp120 core or full-length (envelope) polypeptide variants of the invention or nucleic acids encoding such variants. Thus, the invention includes a recombinant VLP comprising a gp120 polypeptide variant of the invention (or a nucleic acid encoding such variant) and a viral core and/or envelope protein component(s) (e.g., HIV gp41) obtained or derived from a known HIV virus or non-HIV virus.

A recombinant chimeric VLP may be formed, for example, by transfection of a host cell with a bicistronic DNA plasmid vector comprising: (1) a first nucleic acid comprising a nucleotide sequence encoding a gp120 polypeptide variant of the invention (e.g., a nucleotide sequence encoding at least one polypeptide comprising a sequence having at least 85, 90, 91, 92, 93, 94, 95, 96, 97, 98, 99, or 100% sequence identity to a polypeptide sequence selected from any of SEQ ID NO:1-21 and 56-63, or a complementary nucleotide sequence thereof), and (2) a second nucleic acid comprising a nucleotide sequence encoding one or more additional viral core and/or envelope components, including those described herein, such that upon expression of both nucleic acids a recombinant chimeric VLP construct is formed. The DNA plasmid vector may include one expression cassette comprising the first and second nucleic acids or, alternatively, two expression cassettes, with the first expression cassette comprising the first nucleic acid and the second expression cassette comprising the second nucleic acid.

Alternatively, a recombinant chimeric VLP may be formed by transfection of a host cell with two separate monocistronic DNA plasmid vectors, e.g., one vector comprising a nucleic acid comprising a nucleotide sequence encoding a gp120 polypeptide variant of the invention and a second vector comprising a nucleic acid comprising a nucleotide sequence one or more additional viral core and/or envelope components, including those described herein, sufficient to form a VLP when associated with the gp120 polypeptide variant, such that upon expression of both vectors a chimeric VLP is formed.

An exemplary bicistronic vector is a DNA plasmid expression vector comprising a Gag gene of the viral core of an HIV-1 virus (or other viral core or envelope component) covalently fused to a nucleic acid encoding a gp120 core or full-length polypeptide variant of the invention. A chimeric VLP is formed following transfection and expression of such vector in a host cell. The vector may include one expression cassette comprising the Gag gene and gp120 polypeptide variant-encoding nucleic acid or two separate expression cassettes, with the first expression cassette comprising the Gag gene and the second expression cassette comprising the gp120 polypeptide variant-encoding nucleic acid.

Alternatively, a chimeric VLP may be formed by transfecting a host cell with a monocistronic vector comprising the Gag gene and a monocistronic vector comprising a nucleic acid encoding a gp120 polypeptide variant of the invention and co-expressing the Gag gene and gp120 polypeptide variant-encoding nucleic acid in the cell.

In another aspect, the invention includes a chimeric VLP comprising an HBV surface antigen (and/or a nucleic acid encoding such antigen) and at least one gp120 core or full-length polypeptide variant of the invention (and/or a nucleic acid encoding such variant). Such VLP may be formed by transfection and expression in a host cell of a bicistronic DNA plasmid vector comprising a nucleic acid comprising a nucleotide sequence encoding an HBV surface antigen and at one nucleic acid comprising a nucleotide sequence encoding at least one gp120 core or full-length polypeptide variant of the invention. The VLP may alternatively be formed by simultaneous or sequential transfection into a host cell of a monocistronic DNA plasmid vector comprising a nucleic acid comprising a nucleotide sequence encoding an HBV surface antigen and a monocistronic DNA plasmid vector comprising at least one nucleic acid comprising a nucleotide sequence encoding at least one gp120 core or full-length polypeptide variant of the invention and co-expression of both such nucleic acids in the cell.

Standard recombinant techniques and expression systems described herein may be used to express such DNA plasmid vectors or nucleic acid constructs described above which comprise, inter alia, a nucleotide sequence encoding a gp120 polypeptide variant of the invention, thereby generating a chimeric HIV VLP comprising a gp120 polypeptide variant of the invention. Exemplary expression systems are set forth in Doan, L. H. et al., supra. Methods for characterizing the functional properties of a VLP of the invention, such as the ability of such a VLP to induce an immune response (e.g., humoral or cellular immune response) against one or more HIV viruses of the same or of different subtypes include as those described elsewhere herein (see, e.g., the Examples, infra) for characterizing analogous properties of gp120 polypeptide variants of the invention.

Alternatively or additionally, viral core and/or envelope components of non-HIV viruses or VLPs can be used to generate recombinant chimeric VLPs of the invention. Non-HIV viral proteins that are known to form VLPs include, but are not limited to, e.g., influenza virus, vesicular stomatitis virus, Semliki-Forest virus (Notka et al., Biol. Chem. 380:341-52 (1999)), human polyomavirus (Goldmann et al., J. Virol. 73:4465-9 (1999)), rotavirus (Jiang et al., Vaccine 17:1005-13 (1999)), parvovirus (Casal, Biotechnology and Applied Biochemistry 29(2):141-150 (1999)), canine parvovirus (Hurtado et al., J. Virol. 70:5422-9 (1996)), hepatitis E virus (Li et al., J. Virol. 71:7207-13 (1997)), HBV (Doan, L. H et al., supra), and HPV (Doan, L. H et al., supra). Thus, for example, a non-HIV VLP (e.g., HBV VLP) can be engineered using known molecular biology techniques to incorporate a heterologous antigen, such as a gp120 core or full-length polypeptide variant of the invention, thereby producing a recombinant chimeric VLP comprising a gp120 core or full-length polypeptide variant of the invention. Also included is a recombinant nucleic acid or DNA plasmid expression vector that encodes such a VLP construct. The chimeric VLP can be readily produced using standard recombinant techniques and expression systems described herein. See also Doan, L. H. et al., and Crooks et al., both supra.

In another aspect, the invention provides a recombinant chimeric VLP comprising a recombinant modified HIV gp160 protein. The modified gp160 protein comprises a gp160 protein sequence of a known HIV virus (e.g., HIV-1 virus) in which the amino acid sequence corresponding to the HIV gp120 core or full-length envelope protein has been substituted with a recombinant gp120 core or full-length polypeptide variant sequence of the invention, respectively. Also included is a recombinant nucleic acid or plasmid expression vector encoding such modified gp160 protein. Included is a DNA plasmid expression vector comprising a nucleic acid comprising a nucleotide sequence encoding an HIV-1 gp160 protein in which the amino acid sequence corresponding to the HIV-1 gp120 core or full-length envelope protein has been substituted with a gp120 core or full-length polypeptide variant sequence of the invention, respectively. The chimeric VLP can be readily generated from such nucleic acid or expression vector by using standard recombinant techniques and an appropriate expression system described herein. See also Doan, L. H. et al., supra.

In another aspect, the invention provides a chimeric VLP comprising a gp120 core or full-length polypeptide variant of the invention fused to or associated with an HIV (e.g., HIV-1) gp41 polypeptide or another trimer-forming motif). Also included is a DNA plasmid expression vector comprising a nucleic acid sequence encoding such gp120 polypeptide variant and a nucleic acid sequence encoding such HIV gp41 polypeptide or other trimer-forming motif.

In another aspect, the invention provides an isolated or recombinant chimeric HIV VLP comprising: (1) a WT HIV gag polyprotein precursor (Pr55) (e.g., HIV-1 gag Pr55) and (2) a WT gp160 envelope protein of an HIV virus (e.g., HIV-1 virus) in which the amino acid sequence corresponding to the WT gp120 core or full-length envelope polypeptide has been substituted with a gp120 core or full-length polypeptide variant of the invention. Also provided is an isolated or recombinant nucleic acid that encodes (1) a WT HIV gag polyprotein precursor and (2) a WT gp160 envelope protein of an HIV virus (e.g., HIV-1 virus) in which the amino acid sequence corresponding to the WT gp120 core or full-length envelope polypeptide has been substituted with a gp120 core or full-length polypeptide variant of the invention. The recombinant chimeric VLP can be produced by expression of such nucleic acid in an appropriate expression system using standard recombinant techniques described herein. Alternatively, such chimeric VLP can be produced by co-expression in the appropriate expression system of two separate nucleic acids: (1) a recombinant nucleic acid encodes a WT HIV Gag polyprotein precursor and (2) a nucleic acid encoding WT gp160 envelope protein of an HIV virus (e.g., HIV-1 virus) in which the amino acid sequence corresponding to the WT gp120 core or full-length envelope polypeptide has been substituted with a gp120 core or full-length polypeptide variant of the invention. The VLP is formed by association of the resulting polypeptides expressed from both such nucleic acids. See also Doan, L. H. et al., supra.

The invention also includes a DNA plasmid expression vector comprising (1) a nucleic acid sequence encoding an HIV-1 Gag protein and (2) a nucleic acid sequence encoding a modified HIV-1 gp 160 Env protein in which the amino acid sequence corresponding to the gp120 core or full-length envelope protein has been substituted with a gp120 core or full-length polypeptide variant of the invention. Also provided is a DNA plasmid expression vector comprising a nucleic acid sequence encoding an HIV-1 Gag polyprotein precursor (Pr55) and a nucleic acid sequence encoding a gp120 polypeptide variant of the invention. Recombinant chimeric VLPs of the invention can be generated by expression of such nucleic acids or expression vectors in recombinant expression systems using known recombinant techniques described herein. See also Doan, L. H. et al., supra.

Compositions comprising one or more VLPs of the invention and a carrier or excipient are included. Also provided are compositions comprising a carrier or excipient and one or more nucleic acids or plasmid expression vectors, as described above, that upon expression that form a VLP. Such compositions may be pharmaceutical compositions that include a pharmaceutically acceptable carrier or excipient. Additional details regarding such compositions are set forth below.

The invention also contemplates defective, replication incompetent pseudoviruses comprising one or more nucleic acids and/or polypeptides of the invention. Included are recombinant chimeric pseudoviruses comprising at least one gp120 core or full-length polypeptide variant of the invention and/or at least one nucleic acid encoding such variant of the invention. Such pseudoviruses are believed capable of inducing a detectable humoral and/or cellular immune response(s) against at least one HIV virus (e.g., at least one HIV-1 virus) upon administration to a subject and thus are believed useful in the prophylactic and therapeutic treatment methods for inducing immune responses against one or more HIV viruses of the same or different subtypes. Such pseudoviruses are believed to be useful as HIV vaccines and may be used in combination with other HIV vaccine or pharmaceutical approaches to prevent or treat disease associated with HIV infection. Such pseudoviruses can serve as advantageous vehicles for delivery to a subject of one or more nucleic acids and/or polypeptides of the invention. Such pseudoviruses may also referred to in the literature as VLPs. See Crooks et al., supra. Methods for making pseudoviruses incorporating at least one gp120 polypeptide variant of the invention and/or at least one nucleic acid encoding such polypeptide variant are known in the art. Richman et al., Proc. Natl. Acad. Sci. USA, 100:4144-4149 (2003); Frost, S. D. et al., J. Virol. 79:6523-6527 (2005). Methods for delivering and administering such pseudoviruses are described in detail below. See also Doan, L. X. et al, supra; U.S. Pat. No. 5,849,586. Methods for characterizing the functional properties of a pseudovirus of the invention, such as the ability of such a VLP to induce a humoral or cellular immune response against one or more HIV viruses of the same or of different subtypes include those described elsewhere herein (see, e.g., the Examples, infra) for characterizing analogous properties of gp120 polypeptide variants of the invention.

In one aspect, the invention provides a pseudovirus comprising (1) at least one recombinant polypeptide comprising a sequence having at least 85, 90, 91, 92, 93, 94, 95, 96, 97, 98, 99, or 100% sequence identity to a polypeptide sequence selected from any of SEQ ID NO:1-21 and 56-63 and/or (2) at least one recombinant nucleic acid encoding at least one polypeptide comprising a sequence having at least 85, 90, 91, 92, 93, 94, 95, 96, 97, 98, 99, or 100% sequence identity to a polypeptide sequence selected from any of SEQ ID NO:1-21 and 56-63, or a complementary nucleotide sequence thereof, wherein the pseudovirus induces an immune response in a subject to whom an effective amount is administered.

Nucleic acids and plasmid expression vectors encoding such pseudoviruses are included. Compositions comprising a carrier or excipient and one or more such pseudoviruses and/or one or more nucleic acids or plasmid expression vectors that, upon expression, form such pseudoviruses are also contemplated. Such compositions may be pharmaceutical compositions that include a pharmaceutically acceptable carrier or excipient. Further details regarding compositions are set forth below.

A variety of expression systems can be utilized for the production of a VLP or pseudovirus of the invention, including those described above. Exemplary expression systems include baculovirus, vaccinia virus, adenovirus, and yeast expression systems. Plasmid expression vectors can also be used to generate VLPs or pseudoviruses of the invention. Typically, the nucleic acid construct(s) or vector(s) encoding the protein(s) of a VLP or pseudovirus is transfected can be transfected into cells of the chosen expression system and transiently or stably expressed in such cells. Monocistronic or bicistonic vectors can be employed as discussed above. The protein(s) are processed and VLPs or pseudoviruses are assembled. The VLPs or pseudoviruses are typically released into the cellular medium and can be isolated from the medium by ultracentrifugation or other standard techniques. The formation of a VLP or pseudovirus can be detected by any suitable technique known in the art including, for example, electron microscopy technique, dynamic light scattering (DLS), selective chromatographic separation (e.g., ion exchange, hydrophobic interaction, and/or size exclusion chromatographic separation of the VLPs or pseudoviruses), and density gradient centrifugation. Exemplary expression systems, methods for producing VLPs and pseudoviruses, and methods for obtaining and purifying VLPs and pseudoviruses are known in the art. See, e.g., Crook et al., supra; Doan, L. H. et al., supra; U.S. Pat. No. 6,099,847; Kang, C. Y. et al, supra; Akahata, W. et al., supra; Goldmann, C. et al., J. Virol. 73(5):4465-4469 (1999); Richman et al., Proc. Natl. Acad. Sci. USA, 100:4144-4149 (2003); Frost, S. D. et al., J. Virol. 79:6523-6527 (2005).

Methods for administering such DNA plasmid vectors or nucleic acid sequences to subjects for producing VLPs or pseudoviruses in the subject, thereby inducing immune responses in the subject, are the same as those described elsewhere herein for administering a DNA plasmid vector or nucleic acid encoding at least gp120 polypeptide variant of the invention. Additionally, VLPs of the invention can be administered by methods known in the art. See, e.g., Crook et al., Doan, L. H. et al., U.S. Pat. No. 6,099,847, Kang, C. Y. et al., Akahata, W. et al., Goldmann, C. et al., Richman et al., Frost, S. D. et al., all supra.

Expression Hosts

The present invention also provides engineered host cells transduced, transfected or transformed with a vector of the invention (e.g., a cloning vector or expression vector) or a nucleic acid of the invention. The engineered host cells can be cultured in conventional nutrient media modified as appropriate for activating promoters, selecting transformants, or amplifying the nucleic acid of interest. The culture conditions, such as temperature, pH, and the like, are those previously used with the host cell selected for expression, and will be apparent to those skilled in the art and in the references cited herein, including, e.g., Freshney (1994) Culture of Animal Cells, a Manual of Basic Technique, 3^{rd} ed., Wiley-Liss, New York and the references cited therein. Polypeptides of the invention encoded by such vectors or nucleic acids of the invention are expressed in such host cells and can be isolated by standard techniques. For example, polypeptides released into the cell culture can be isolated from the culture by ultracentrifugation or similar techniques.

The polypeptides of the invention can be produced in a variety of expression hosts, including, but not limited to, animal cells, such as mammalian cells (e.g., CHO cells), including human and non-human primate cells, and in non-animal cells, such as plants, yeast, fungi, bacteria, and the like. Examples of suitable expression hosts include bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells, such as CHO (e.g., CHO-K1), COS (e.g., COS-1, COS-7), BHK, and HEK (e.g., HEK 293) cells, Bowes melanoma cells, and plant cells. As noted above, the invention is not limited by the host cells employed.

In addition to Sambrook, Berger and Ausubel, all supra, details regarding cell culture are found in, e.g., Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg NY); Atlas & Parks (eds.) The Handbook of Microbiological Media (1993) CRC Press, Boca Raton, Fla.

The invention provides a cell(s) comprising any one or more of the nucleic acids, vectors, or other constructs of the invention (e.g., a construct expressing a gp120 polypeptide variant) described herein or any combination thereof. Also included is a cell comprising one or more of any of the polypeptides, antibodies, or fusion proteins, or other constructs of the invention described herein, or any combination of one or more of these. A cell of the invention is typically an isolated or recombinant cell and may comprise a host cell. Such a cell, e.g., recombinant cell, may be modified by transformation, transfection, and/or infection with at least one nucleic acid, vector, or other construct of the invention. Such a cell can be a eukaryotic cell (e.g., mammalian, yeast, or plant cell) or a prokaryotic cell (e.g., bacterial cell) and can be transformed with any such construct of the invention using a variety of known methods, including, e.g., calcium phosphate transfection (see, e.g., calcium phosphate co-precipitation method), DEAE-Dextran mediated transfection, electroporation (Irving et al., Cell 64:891-901 (1991)), gene or vaccine gun, injection, lipofection and biolistics or other common techniques as noted above. See also Inovio Biomedical Corp. electroporation methods and technology at the website address inovio.com.

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing that cleaves a “pre” or a “prepro” form of the protein may also be important for correct insertion, folding and/or function of the polypeptide, as discussed above, which in the case of many of the antigenic and/or immunogenic amino acid sequences of the invention can be cell type-dependent. Different host cells such as E. coli, Bacillus sp., yeast, or mammalian cells, such as CHO, HeLa, BHK, MDCK, HEK 293, WI38, etc. have specific cellular machinery and characteristic mechanisms for such post-translational activities and may be chosen to ensure the correct modification and processing of the introduced foreign protein.

A nucleic acid of the invention can be inserted into an appropriate host cell (in culture or in a host organism) to permit the host to express the protein of interest (e.g., a recombinant gp120 full-length or core polypeptide variant). Any suitable host cell can be used transformed/transduced by the nucleic acids of the invention. Examples of appropriate expression hosts include: bacterial cells, such as E. coli, Streptomyces, Bacillus sp., and Salmonella typhimurium; fungal cells, such as Saccharomyces cerevisiae, Pichia pastoris, and Neurospora crassa; insect cells such as Drosophila and Spodoptera frugiperda; mammalian cells such as Vero cells, HeLa cells, CHO cells (e.g., CHO-K1), COS cells, WI38 cells, NIH-3T3 cells (and other fibroblast cells, such as MRC-5 cells), MDCK cells, KB cells, SW-13 cells, MCF7 cells, BHK cells, HEK-293 cells, Bowes melanoma cells, and plant cells, etc. For example, a nucleic acid of the invention can be transformed into dicot plant cells by way of a Ti or Ri plasmid in a suitable bacterial vector (e.g., an Agrobacterium tumefaciens bacterial vector), which cells can be in a live plant, an explant, suitable protoplast cells, or other appropriate plant culture. Dicot cells are typically transformed by PEG and/or CaPO₄-mediate transfection and other known techniques (see generally Potrykus, Ciba Found Symp. 154:198-212 (1990)). Techniques for ensuring appropriate glycosylation have been developed with mammalian antibodies (i.e., so-called “plantbodies,” which can generally be applied to polypeptides and antibodies of the invention (with the recognition that some minor differences in glycosylation, such as fructose linkages, will be present in such polypeptides) (see, e.g., Ma et al., Nature Med. 4:601-606 (1998), Cabanes-Macheteau et al. (1999) Glycobiology. 9(4):365-72, Chargelegue et al. (2000), Transgenic Res. 9:187-94, and Khoudi et al. (1999) Biotechnology Bioeng. 64:135-43). It is understood that not all cells or cell lines need to be capable of producing fully functional polypeptides or fragments thereof; for example, antigenic fragments of the polypeptide may be produced in a bacterial or other non-glycosylating and/or non-proteolytic cleaving expression system. Additional examples of suitable host cells are described, for example, in U.S. Pat. No. 5,994,106 and Int'l Patent Application WO 95/34671.

The present invention also provides host cells that are transduced, transformed or transfected with at least one nucleic acid or vector of the invention. As discussed above, a vector of the invention typically comprises a nucleic acid of the invention. Host cells are genetically engineered (e.g., transduced, transformed, infected, or transfected) with the vectors of the invention, which may be, for example, a cloning vector or an expression vector. The vector may be, for example, in the form of a plasmid, a viral particle, a phage, attenuated bacteria, or any other suitable type of vector. Host cells suitable for transduction and/or infection with viral vectors of the invention for production of the recombinant polypeptides of the invention and/or for replication of the viral vector of the invention include the above-described cells.

Examples of cells that have been demonstrated as suitable for packaging of viral vector particles are described in, e.g., Inoue et al., J. Virol. 72(9):7024-31 (1998), Polo et al., Proc. Natl. Acad. Sci. 96(8):4598-603 (1999), Farson et al., J. Gene Med. 1(3):195-209 (1999), Sheridan et al., Mol. Ther. 2(3):262-75 (2000), Chen et al., Gene Ther. 8(9):697-703 (2001), and Pizzaro et al., Gene Ther. 8(10):737-745 (2001). For replication-deficient viral vectors, such as AAV vectors, complementing cell lines, or cell lines transformed with helper viruses, or cell lines transformed with plasmids encoding essential genes, are necessary for replication of the viral vector.

The engineered host cells can be cultured in conventional nutrient media modified as appropriate for activating promoters, selecting transformants, or amplifying the gene of interest. The culture conditions, such as temperature, pH, and the like, are those previously used with the host cell selected for expression, and will be apparent to those skilled in the art and in the references cited herein, including, e.g., ANIMAL CELL TECHNOLOGy, Rhiel et al., eds., (Kluwer Academic Publishers 1999), Chaubard et al., Genetic Eng. News 20(18) (2000), Hu et al., ASM News 59:65-68 (1993), Hu et al., Biotechnol. Prog. 1:209-215 (1985), Martin et al., Biotechnol. (1987), Freshney, CULTURE OF ANIMAL CELLS: A MANUAL OF BASIC TECHNIQUE, 4^{th} ed., (Wiley, 2000), Mather, INTRODUCTION TO CELL AND TISSUE CULTURE: THEORY AND TECHNIQUE, (Plenum Press, 1998), Freshney, CULTURE OF IMMORTALIZED CELLS, 3^{rd} ed., (John Wiley & Sons, 1996), CELL CULTURE: ESSENTIAL TECHNIQUES, Doyle et al., eds. (John Wiley & Sons 1998), and GENERAL TECHNIQUES OF CELL CULTURE, Harrison et al., eds., (Cambridge Univ. Press 1997). The nucleic acid also can be contained, replicated, and/or expressed in plant cells. Techniques related to the culture of plant cells are described in, e.g., Payne et al. (1992) PLANT CELL AND TISSUE CULTURE IN LIQUID SYSTEMS John Wiley & Sons, Inc. New York, N.Y.; Gamborg and Phillips (eds.) (1995) PLANT CELL, TISSUE AND ORGAN CULTURE: FUNDAMENTAL METHODS SPRINGER LAB MANUAL, Springer-Verlag (Berlin Heidelberg New York) and Plant Molecular Biology (1993) R. R. D. Croy (ed.) Bios Scientific Publishers, Oxford, U.K. ISBN 0 12 198370 6. Cell culture media in general are set forth in Atlas and Parks (eds.) THE HANDBOOK OF MICROBIOLOGICAL MEDIA (1993) CRC Press, Boca Raton, Fla.

For long-term, high-yield production of recombinant proteins, stable expression systems can be used. For example, cell lines that stably express a polypeptide of the invention can be transduced with expression vectors comprising viral origins of replication and/or endogenous expression elements and a selectable marker gene. Following the introduction of the vector, cells in the cell line may be allowed to grow for 1-2 days in an enriched media before they are switched to selective media. The purpose of the selectable marker is to confer resistance to selection, and its presence allows growth and recovery of cells that successfully express the introduced sequences. For example, resistant clumps of stably transformed cells can be proliferated using tissue culture techniques appropriate to the cell type.

Host cells transformed with an expression vector and/or polynucleotide are optionally cultured under conditions suitable for the expression and recovery of the encoded protein from cell culture. The polypeptide or fragment thereof produced by such a recombinant cell may be secreted, membrane-bound, or contained intracellularly, depending on the sequence and/or the vector used. Expression vectors comprising polynucleotides encoding mature polypeptides of the invention can be designed with signal sequences (e.g., tPA signal peptide, SEQ ID NO:52) that direct secretion of the mature polypeptides through a prokaryotic or eukaryotic cell membrane. Such signal sequences are typically incorporated into the vector such that the signal sequence is expressed at the N-terminus of the polypeptide of the invention. Principles related to such signal sequences are discussed elsewhere herein. Expression systems useful for production of VLPs of the invention are described in detail in Doan, L. H. et al, supra.

Polypeptide Production and Recovery

Following transduction of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

As noted, many references are available for the culture and production of many cells, including cells of bacterial, plant, animal (especially mammalian) and archebacterial origin. See e.g., Sambrook, Ausubel, and Berger (all supra), as well as Freshney (1994) Culture of Animal Cells, a Manual of Basic Technique, Third edition, Wiley-Liss, New York and the references cited therein; Doyle and Griffiths (1997) MAMMALIAN CELL CULTURE: ESSENTIAL TECHNIQUES John Wiley and Sons, NY; Humason (1979) Animal Tissue Techniques, fourth edition W.H. Freeman and Company; and Ricciardelli, et al., (1989) In vitro Cell Dev. Biol. 25:1016 1024. For plant cell culture and regeneration, Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Plant Molecular Biology (1993) R. R. D. Croy, Ed. Bios Scientific Publishers, Oxford, U.K. ISBN 0 12 198370 6. Cell culture media in general are set forth in Atlas and Parks (eds.) The Handbook of Microbiological Media (1993) CRC Press, Boca Raton, Fla. Additional information for cell culture is found in available commercial literature such as the Life Science Research Cell Culture Catalogue (1998) from Sigma-Aldrich, Inc. (St. Louis, Mo.) (“Sigma-LSRCCC”) and, e.g., the Plant Culture Catalogue and supplement (1997) also from Sigma-Aldrich, Inc (St. Louis, Mo.) (“Sigma-PCCS”).

Polypeptides of the invention can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted supra, a variety of purification methods are well known in the art, including, e.g., those set forth in Sandana (1997) Bioseparation of Proteins, Academic Press, Inc.; and Bollag et al. (1996) Protein Methods, 2.sup.nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3.sup.rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

In Vitro Expression Systems

Cell-free transcription/translation systems can also be employed to produce recombinant polypeptides of the invention or fragments thereof using DNAs and/or RNAs of the present invention or fragments thereof. Several such systems are commercially available. A general guide to in vitro transcription and translation protocols is found in Tymms (1995) IN VITRO TRANSCRIPTION AND TRANSLATION PROTOCOLS: METHODS IN MOLECULAR BIOLOGY, Volume 37, Garland Publishing, New York.

Additional Sequences of the Invention

In another aspect, the invention provides a nucleic acid comprising a first nucleotide sequence encoding at least one polypeptide of the invention and a second nucleotide sequence that is an immunostimulatory sequence, e.g., a sequence according to the sequence pattern (N₁CGN₂)ₓ, wherein N₁ is, 5′ to 3′, any two purines, any purine and a guanine, or any three nucleotides; N₂ is, 5′ to 3′, any two purines, any guanine and any purine, or any three nucleotides; and x is any number greater than 0. Immunomodulatory sequences are known in the art, and described in, e.g., Wagner et al. (2000) Springer Semin. Immunopathol. 22(1-2):147-52, Van Uden et al. (2000) Springer Semin Immunopathol 22(1-2):1-9, and Pisetsky (1999) Immunol. Res. 19(1):35-46, as well as U.S. Pat. Nos. 6,207,646, 6,194,388, 6,008,200, 6,239,116, and 6,218,371. Other immunostimulating unmethylated CpG motifs in immunostimulatory sequences are known, and it is recognized that particular motifs are effective in particular host and/or host cells.

In another aspect, the invention provides a nucleic acid that comprises a first polynucleotide sequence that encodes at least one recombinant polypeptide of the invention and further comprises a second polynucleotide sequence that encodes at least one protein adjuvant. Such nucleic acid may be an expression vector. Alternatively, the invention provides two nucleic acids that are administered separately, with the first nucleic acid comprising a polynucleotide sequence that encodes at least one polypeptide of the invention, and the second nucleic acid comprising a polynucleotide sequence that encodes a protein adjuvant. Each such nucleic acid may be an expression vector. The adjuvant that promotes the immune response that is induced by at least one recombinant polypeptide of the invention may be a cytokine, such as a granulocyte macrophage colony stimulating factor (a GM-CSF, e.g., a human GM-CSF) an interferon (e.g., human interferon (IFN) alpha, IFN-beta, IFN-γ), an Interleukin (e.g., an IL-2, IL-12, IL-15, IL-18, etc.). Genes encoding such cytokines are known. Human GM-CSF sequences are described in, e.g., Wong et al. (1985) Science 228:810, Cantrell et al. (1985) Proc. Natl. Acad. Sci. 82:6250, and Kawasaki et al. (1985) Science 230:291. In one embodiment, such a nucleic acid expresses an amount of GM-CSF or a functional analog thereof that detectably stimulates the mobilization and differentiation of dendritic cells (DCs) and/or T-cells, increases antigen presentation, and/or increases monocytes activity, such that the immune response induced by the immunogenic recombinant polypeptide of the invention is increased. Suitable interferon genes, such as IFN-γ genes also are known (see, e.g., Taya et al. (1982), EMBO J. 1:953-958, Cerretti et al. (1986) J. Immunol. 136(12):4561, and Wang et al. (1992) Sci. China. B. 35(1):84-91). The IFN, such as the IFN-γ, is expressed from the nucleic acid in an amount that increases the immune response of the immunogenic recombinant polypeptide of the invention (e.g., by enhancing a T cell immune response induced by the immunogenic polypeptide). Advantageous IFN-homologs and IFN-related molecules that can be co-expressed or co-administered with a polynucleotide and/or polypeptide of the invention are described in, e.g., International Patent Application Publications WO 01/25438 and WO 01/36001. Co-administration (which herein includes both simultaneous and serial administration) of about 1 to 5 to about 10 μg of a GM-CSF-encoding plasmid with about 5 to about 50 μg of a plasmid encoding one of the polypeptides of the invention is expected to be effective or useful for enhancing the antibody response in a mouse model. In another aspect, co-administration of about 1 μg to about 1 mg, 10 μg to about 500 μg, 100 μg to about 250 μg, 10 μg to about 100 μg of a GM-CSF-encoding plasmid with, respectively, an amount of 5 μg to about 5 mg, 50 μg to about 2.5 mg, 500 μg to about 1 mg, 50 μg to about 1 mg of a plasmid encoding one of the polypeptides of the invention may be effective for enhancing the antibody response in a mouse model.

Methods of the Invention

Polypeptides, nucleic acids, vectors, viruses, virus-like particles (VLPs), and pseudoviruses of the invention exhibit a variety of properties and characteristics and may be useful in a variety of contexts, including in prophylactic or therapeutic methods that generally involve inducing an immune response against at least one HIV virus in a subject in need thereof by administering to the subject an amount of such polypeptide, nucleic acid, vector, virus or VLP effective to induce the immune response. In a therapeutic context, the subject is typically one infected with an HIV virus (e.g., an HIV-1 virus). In a prophylactic context, the subject has not been previously infected with an HIV virus (e.g., an HIV-1 virus), and administration is conducted to prevent infection should the subject come in contact with an HIV virus. A polypeptide, nucleic acid, vector, virus, VLP, or pseudovirus (pseudovirion) of the invention may be able to induce or promote an immune response against at least one HIV virus (e.g., HIV-1) or pseudovirus in such a subject (including, e.g., a mammal, such as a human) or in a population of cells. The term “induce” or “promote” encompasses any detectable increase in an existing immune response in the subject to whom an effective amount of such a polypeptide, nucleic acid, vector, virus, VLP, or pseudovirion is administered. Immune responses include, for example, one or more of the following: the induction or production of one or more antibodies against at least one HIV (e.g., HIV-1) virus or pseudovirus; a T cell response, such as, e.g., T cell activation or proliferation response; the priming and stimulation of CD4+ and/or CD8+ lymphocytes; the production of one or more cytokines, including the production of one or more tumor necrosis factors (TNF), such as, e.g., TNF-alpha; the production of one or more interleukins (IL), such as, e.g., IL-1, IL-2, IL-3, IL-4, IL-5, IL-6, IL-10, IL-12; the production of one or more interferons (IFN), such as, e.g., IFN-gamma, IFN-alpha, and IFN-beta; complement activation; platelet activation; an enhanced and/or decreased Th1 response; and an enhanced and/or decreased Th2 response. Such immune response may be against multiple HIV (e.g., HIV-1) viruses or pseudoviruses of the same subtype or of different subtypes. For example, a recombinant polypeptide or nucleic acid of the invention may induce the production of antibodies against 1, 2, 3, 4, 5, 6, 7, 8, 9, 10 or more HIV-1 viruses or pseudoviruses in a subject to whom an effective amount of the polypeptide or nucleic acid is administered.

An antibody response induced in a subject by a polypeptide, nucleic acid, vector, virus, VLP, or pseudovirion of the invention to whom an effective amount of such molecule is administered may comprise a detectable neutralizing antibody response against one or more HIV-1 viruses or pseudoviruses of the same subtype or of different subtypes. Antibodies induced by a polypeptide, nucleic acid, vector, virus, VLP, or pseudovirus of the invention may be characterized by an ability to bind at least one HIV-1 virus or pseudovirus.

The invention includes a method of inducing an immune response against at least one HIV virus (e.g., HIV-1 virus) in a subject in need thereof, which comprises administering to the subject at least one of the following in an amount effective to induce or promote a detectable immune response against at least one HIV virus (e.g., HIV-1 virus) in the subject: 1) at least one nucleic acid of the invention; 2) at least one vector (e.g., plasmid vector) comprising at least one nucleic acid of the invention; 3) at least one polypeptide of the invention; 4) at least one virus of the invention comprising at least one nucleic acid and/or polypeptide of the invention; 5) at least one VLP or pseudovirion of the invention comprising at least one polypeptide of the invention; 6) at least one viral vector of the invention comprising at least one nucleic acid and/or polypeptide of the invention; 7) at least one composition of the invention comprising an excipient or carrier and at least one nucleic acid or polypeptide of the invention; or any combination thereof of any of the foregoing. The effective amount is typically that amount sufficient to induce a detectable humoral or cellular immune response against the at least one HIV virus (e.g., HIV-1 virus). The subject may be an animal, including a mammal, including, e.g., but not limited to a non-human primate or human.

Depending on the context, the amount of the polypeptide, nucleic acid, vector, virus, VLP, or pseudovirion (i.e., entity) administered to a subject may be a prophylactically effective amount or a therapeutically effective amount of such entity. A prophylactically effective amount of such entity may be an amount of the entity effective to inhibit or prevent HIV (e.g., HIV-1) infection in the subject not previously infected with HIV upon initial exposure to the HIV virus. A therapeutically effective amount of an entity may be an amount effective to inhibit or reduce HIV infection in a subject who has been exposed or infected with an HIV virus or to whom an HIV virus has been transmitted—prior to administration of the polypeptide, nucleic acid, vector, virus, VLP, or pseudovirion.

For some such methods, the effective amount of such polypeptide, nucleic acid, vector, virus, VLP, or pseudovirion administered to a subject is typically an amount sufficient to induce a detectable humoral or cellular immune response in the subject. A humoral immune response may comprise the production of neutralizing antibodies against at least one HIV virus (e.g., HIV-1) and/or an HIV-specific (e.g., HIV-1) T cell response. The immune response may comprise the production of neutralizing antibodies and/or a virus-specific effector T cell response against at least 1, 2, 3, 4, 5, 6, 7, 8, 9, 10 or more HIV viruses of the same subtype or of different subtypes or clades. In one aspect, the immune response may comprise the production of neutralizing antibodies or a virus-specific T cell response against at 1, 2, 3, 4, 5, 6, 7, 8, 9, 10 or more HIV-1 viruses of the same subtype (e.g., subtype B) or of different subtypes or clades (e.g., A, B, C, D, F, G, H, and J).

In one aspect, the invention includes a method of inducing an immune response against at least one HIV-1 virus or HIV-1 pseudovirus in a subject (e.g., a mammal), which comprises administering to the subject at least one nucleic acid having at least 85, 86, 87, 88, 89, 90, 91, 92, 93, 94, 95, 96, 97, 98, 99, or 100% sequence identity to a nucleotide sequence selected from the group consisting of SEQ ID NOS:23-50 and 64-79 in an amount that is effective to induce in the subject an immune response against at least one HIV-1 virus or HIV-1 pseudovirus. The nucleic acid may comprise part of a plasmid vector, viral vector, or virus that is administered to the subject. The effective amount of the nucleic acid is typically an amount sufficient to induce a detectable immune response in the subject, and the immune response may be against one or more HIV-1 viruses or pseudoviruses of the same or different subtypes. In particular, the induced immune response may comprise an anti-HIV-1 neutralizing antibody response or HIV-1-specific T cell immune response or both. In some instances, the immune response may comprise an anti-HIV-1 neutralizing antibody response or HIV-1-specific T cell immune response (or both such responses) against 1, 2, 3, 4, 5, 6, 7, 8, or more HIV-1 viruses or pseudoviruses of the same subtype, of different subtypes, or any combination of subtypes.

Also provided is a method of inducing an immune response against at least one HIV-1 virus or pseudovirus in a subject (e.g., a mammal), which comprises administering to the subject at least one polypeptide having at least 85, 86, 87, 88, 89, 90, 91, 92, 93, 94, 95, 96, 97, 98, 99, or 100% sequence identity to a polypeptide sequence selected from the group consisting of SEQ ID NOS:1-21 and 56-63 in an amount that is effective to induce in the subject an immune response against at least one HIV-1 virus or pseudovirus. The polypeptide may comprise part of a virus or VLP that is administered to the subject. The effective amount of the nucleic acid is typically an amount sufficient to induce a detectable immune response in the subject, and the immune response may be against one or more HIV-1 viruses or pseudoviruses of the same or different subtypes. In particular, the induced immune response may comprise an anti-HIV-1 neutralizing antibody response or HIV-1-specific T cell immune response or both. In some instances, the immune response may comprise an anti-HIV-1 neutralizing antibody response or HIV-1-specific T cell immune response (or both such responses) against 1, 2, 3, 4, 5, 6, 7, 8, or more HIV-1 viruses or pseudoviruses of the same subtype, of different subtypes, or any combination of subtypes.

Also included is a prophylactic treatment method of preventing HIV-1 infection in a subject in need thereof, which comprises administering to the subject prior to exposure to an HIV-1 virus at least one polypeptide, nucleic acid, vector, virus, pseudovirus VLP, or composition of the invention, or any combination of any thereof, in a prophylactically effective amount that prevents HIV-1 infection in the subject. The HIV-1 virus inoculum size (initial HIV-1 viral dose) to which the subject is subsequently exposed may be reduced such that total HIV-1 viral load and/or viral set point is reduced, thereby inhibiting or blunting HIV-1 infection, as compared to the HIV-1 viral load and/or set point that would be reached without prior administration of such polypeptide, nucleic acid, vector, virus, pseudovirus, VLP, or composition. In some such methods, the initial dose of HIV-1 virus (inoculum size) may be reduced in the subject by at least 10%, 20%, 30%, 50%, 60% 70%, 80%, 90%, 95%, 98%, 99%, or 100%.

Also provided is a therapeutic treatment method of inhibiting or reducing HIV-1 infection in a subject previously exposed to an HIV-1 virus or suffering from an HIV-1 infection, which comprises administering to the subject at least one polypeptide, nucleic acid, vector, virus, pseudovirus, VLP, or composition of the invention, or any combination of any thereof, in a therapeutically effective amount that inhibits or reduces HIV-1 infection in the subject. An HIV-1 humoral and/or HIV-1-specific T cell response may be sufficiently elicited such that the HIV-1 infection or HIV-1 viral load is inhibited or reduced in the subject. In some such methods, the HIV-1 infection or viral load may be reduced in the subject by at least 10%, 20%, 30%, 50%, 60%, 70%, 80%, 90%, 95%, 98%, 99%, or more.

Also included is a method of reducing the dose of a HIV-1 virus transmitted (inoculum size) to a subject following exposure to such HIV-1 virus, which comprises administering to the subject at least one polypeptide, nucleic acid, vector, virus, or VLP in a therapeutically effective amount that reduces the dose or inoculum size of HIV-1 virus transmitted to the subject. For some such methods, the therapeutically effective amount may be sufficient to reduce the inoculum size by as much as 5%, 10%, 20%, 30%, 50%, 60% 70%, 80%, 90% or more. An HIV-1 specific humoral and/or T cell response may be elicited that reduces the HIV-1 virus inoculum size.

The invention also provides a method of preventing a disease associated with HIV-1 infection in a subject in need thereof. The method comprises administering to the subject an amount of at least one polypeptide, nucleic acid, vector, virus, pseudovirus, VLP, or composition of the invention, or any combination of any thereof, that is effective amount to prevent the disease.

Also provided is a method of inducing an immune response against at least one HIV-1 virus in a mammal, which comprises administering to the mammal: (1) at least one nucleic acid that encodes an HIV-1 92US657 gp120 full-length or core polypeptide, (2) at least one vector comprising a nucleic acid that encodes an HIV-1 92US657 gp120 full-length or core polypeptide, (3) at least one HIV-1 92US657 gp120 full-length or core polypeptide, (4) at least one virus or virus-like particle that comprises such an HIV-1 92US657 gp120 polypeptide or nucleic acid, and/or (5) at least one cell comprising such an HIV-1 92US657 gp120 polypeptide or nucleic acid, in an amount effective to induce in the subject a detectable immune response against the at least one HIV-1 virus, such as, e.g., the production of neutralizing antibodies against HIV-1 or an HIV-1 specific T cell response. See GenBank No. AAB05049 for the genome of HIV-1 virus 92US657. The nucleic acid encoding the gp120 Env protein of HIV-1 virus 92US657 can be cloned from the genome using methods analogous to those described elsewhere with regard to the cloning of parental HIV-1 gp120 gene sequences. Alternatively, the nucleic acid gp120 Env protein of HIV-1 virus 92US657 can be synthesized using standard synthesis techniques. The encoded 92US657 gp120 polypeptide can also be produced using methods analogous to those described elsewhere for producing parental gp120 polypeptides.

The invention provides a method of reducing or inhibiting HIV-1 infection or transmission in a subject in need thereof, which comprises administering to the subject at least one HIV-1 92US657 gp120 full-length or core polypeptide or nucleic acid encoding such polypeptide in an amount is effective to reduce or inhibit HIV-1 infection or transmission in the subject.

If desired, any method discussed above may further comprise administration to the subject of at least one adjuvant, co-stimulatory molecule, antigen, and/or cytokine (or nucleic acid encoding any of these) in an amount sufficient to enhance the immune response. Additionally, if desired, in any method described above, the polypeptide, nucleic acid, vector, virus, or VLP of the invention (or any combination thereof) may be administered in a composition comprising a carrier or excipient and such polypeptide, nucleic acid, vector, virus, or VLP. The composition may be a pharmaceutical composition comprising a pharmaceutically acceptable carrier or excipient and such polypeptide, nucleic acid, vector, virus, or VLP.

Immune responses induced by a polypeptide, nucleic acid, vector, virus, pseudovirus, VLP, or composition of the invention can be measured by any suitable technique. Examples of useful techniques in assessing humoral immune responses include flow cytometry, immunoblotting assays, immunohistochemistry assays, immunoprecipitation assays, radioimmunoassays (RIA), and enzyme immunoassays. Enzyme immunoassays include enzyme-linked immunoflow assays (ELIFA) and enzyme-linked immunosorbent assays (ELISA), including sandwich ELISA and competitive ELISA assays. ELISA assays involve the reaction of a specific (first) antibody with an antigen. The resulting antibody-antigen complex is detected by using a second antibody against the first antibody. The second antibody is enzyme-labeled, and an enzyme-mediated color reaction is produced by reaction with the first antibody. Suitable antibody labels for such assays include radioisotopes; enzymes, such as horseradish peroxidase (HRP) and alkaline phosphatase (AP), biotin, and fluorescent dyes, such as fluorescein or rhodamine. HPLC and capillary electrophoresis (CE) also can be utilized in immunoassays to detect complexes of antibodies and target substances. General guidance performing such techniques and related principles are described in, e.g., Harlow and Lane (1988) ANTIBODIES, A LABORATORY MANUAL, Cold Spring Harbor Publications, New York, Hampton R et al. (1990) SEROLOGICAL METHODS A LABORATORY MANUAL, APS Press, St. Paul Minn., Stevens (1995) CLINICAL IMMUNOLOGY AND SEROLOGY: A LABORATORY PERSPECTIVE, CRC press, Bjerrum (1988) HANDBOOK OF IMMUNOBLOTTING OF PROTEINS, Vol. 2, Zoa (1995) DIAGNOSTIC IMMUNOPATHOLOGY: LABORATORY PRACTICE AND CLINICAL APPLICATION, Cambridge University Press, Folds (1998) CLINICAL DIAGNOSTIC IMMUNOLOGY: PROTOCOLS IN QUALITY ASSURANCE AND STANDARDIZATION, Blackwell Science Inc., Bryant (1992) LABORATORY IMMUNOLOGY & SEROLOGY 3^{rd} edition, W B Saunders Co., and Maddox D E et al. (1983) J. Exp. Med. 158:1211. Guidance with respect to ELISA techniques and related principles are described in, e.g., Reen (1994) Methods Mol. Biol. 32:461-6, Goldberg et al. (1993) Curr. Opin. Immunol. 5(2):278-81, Voller et al. (1982) Lab. Res. Methods Biol. Med. 5:59-81, Yolken et al. (1983) Ann. NY Acad. Sci. 420:381-90, Vaughn et al. (1999) Am. J. Trop. Med. Hyg. 60(4):693-8, and Kuno et al. (1991) J. Virol. Methods 33(1-2):101-13. Guidance with respect to flow cytometry techniques is provided in, e.g., Diamond (2000) IN LIVING COLOR: PROTOCOLS IN FLOW CYTOMETRY AND CELL SORTING, Springer Verlag, Jaroszeki (1998) FLOW CYTOMETRY PROTOCOLS, 1^{st} Ed., Shapiro (1995) PRACTICAL FLOW CYTOMETRY, 3^{rd} edition, Rieseberg et al. (2001) Appl. Microbiol. Biotechnol. 56(3-4):350-60, Scheffold and Kern (2000) J. Clin. Immunol. 20(6):400-7, and McSharry (1994) Clin. Microbiol. Rev. (4):576-604.

Cytotoxic and other T cell immune responses also can be measured by any suitable technique. Examples of such techniques include ELISpot assay (particularly, IFN-gamma ELISpot), intracellular cytokine staining (ICC) (particularly in combination with FACS analysis), CD8+ T cell tetramer staining/FACS, standard and modified T cell proliferation assays, chromium release CTL assay, limiting dilution analysis (LDA), and CTL killing assays. Guidance and principles related to T cell proliferation assays are described in, e.g., Plebanski and Burtles (1994) J. Immunol. Meth. 170:15, Sprent et al. (2000) Philos. Trans. R. Soc. Lond. B Biol. Sci. 355(1395):317-22 and Messele et al. (2000) Clin. Diagn. Lab. Immunol. 7(4):687-92. LDA is described in, e.g., Sharrock et al. (1990) Immunol. Today 11:281-286. ELISpot assays and related principles are described in, e.g., Czerinsky et al. (1988) J. Immunol. Meth. 110:29-36, Olsson et al. (1990) J. Clin. Invest. 86:981-985, Schmittel et al. (2001) J. Immunol. Meth. 247(1-2):17-24, Ogg and McMichael (1999) Immunol. Lett. 66(1-3):77-80, Schmittel et al. (2001) J. Immunol. Meth. 247(1-2):17-24, Kurane et al. (1989) J. Exp. Med. 170(3):763-75, Chain et al. (1987) J. Immunol. Meth. 99(2):221-8, Czerkinsky et al. (1988) J. Immunol. Meth. 110:29-36, and U.S. Pat. Nos. 5,750,356 and 6,218,132. Tetramer assays are discussed in, e.g., Skinner et al. (2000) J. Immunol. 165(2):613-7. Other T cell analytical techniques are described in Hartel et al. (1999) Scand. J. Immunol. 49(6):649-54 and Parish et al. (1983) J. Immunol. Meth. 58(1-2):225-37.

T cell activation also can be analyzed by measuring CTL activity or expression of activation antigens such as IL-2 receptor, CD69 or HLA-DR molecules. Proliferation of purified T cells can be measured in a mixed lymphocyte culture (MLC) assay. MLC assays are known in the art. Briefly, a mixed lymphocyte reaction (MLR) is performed using irradiated peripheral blood mononuclear cells (PBMC) as stimulator cells and allogeneic PBMC as responders. Stimulator cells are irradiated (2500 rads) and co-cultured with allogeneic PBMC (1×10⁵ cells/well) in 96-well flat-bottomed microtiter culture plates (VWR) at 1:1 ratio for a total of 5 days. During the last 8 hours of the culture period, the cells were pulsed with 1 uCi/well of ³H-thymidine, and the cells are harvested for counting onto filter paper by a cell harvester as described above. ³H-thymidine incorporation is measured by standard techniques. Proliferation of T cells in such assays is expressed as the mean cpm read for the tested wells.

ELISpot assays measure the number of T-cells secreting a specific cytokine, such as interferon-gamma or tumor necrosis factor-alpha, that serves as a marker of T-cell effectors. Cytokine-specific ELISA kits are commercially available (e.g., an IFN-gamma-specific ELISPot is available through R&D Systems, Minneapolis, Minn.).

Production and expression of recombinant polypeptides of the invention can be assessed by using any suitable technique, such as a Western blot assay. Guidance with respect to Western blot techniques can found in, e.g., Ausubel et al., CURRENT PROTOCOLS IN MOLECULAR BIOLOGY (Wiley Interscience Publishers, 1995). Specific exemplary applications of Western blot techniques can be found in, e.g., Churdboonchart et al. (1990) Southeast Asian J. Trop. Med. Public Health 21(4):614-20 and Dennis-Sykes et al. (1985) J. Biol. Stand. 13(4):309-14.

A recombinant polypeptide or nucleic acid of the invention may have the ability to induce a neutralizing antibody response against an HIV-1 virus or pseudovirus. Neutralizing antibodies interfere with HIV-1 infection by binding to virus envelope proteins and perturbing one or more steps in viral attachment and entry Burton et al., Curr. Top. Microbiol. Immunol. 260:109-143 (2001). Neutralizing antibodies are thus analogous to entry inhibitors. Neutralizing antibody responses can be characterized using a virus neutralization assay. Various viral neutralization assays are known in the art (see, e.g., Richman et al., Proc. Natl. Acad. Sci. USA, 100:4144-4149 (2003); Frost, S. D. et al., J. Virol. 79:6523-6527 (2005)) and can be employed to assess the ability of a gp120-encoding nucleic acid variants or gp120 polypeptide variant of the invention to induce a neutralizing antibody response against one or more HIV-1 viruses, including one or more HIV-1 viruses of the same or different subtypes. An exemplary assay for characterizing a neutralizing antibody response to HIV-1 is provided in Richman et al., Proc. Natl. Acad. Sci. USA 100(7):4144-4149. See also Petropoulos et al., Antimicrob. Agents Chemother. 44:920-928 (2002); Parkin et al., Antimicrob. Agents Chemother. 48:437-443 (2004). Assays can be configured to measure the activity of neutralizing antibodies in blood or other tissue/fluid samples. Virus panels can include those HIV-1 viruses that are typically found in humans in early infection (see, e.g., Richman et al., supra) and thus represent viruses against which a prophylactic vaccine would be useful.

An exemplary assay for measuring the neutralizing antibody response induced in a subject (e.g., mammal) by a polypeptide or nucleic acid of the invention or against an HIV-1 pseudovirus derived from patient samples (e.g. plasma, CSF) or from well-characterized laboratory strains (e.g., HXB2) is described in an Example 6 below. This assay is useful in measuring cross-neutralizing antibody responses induced by a polypeptide or nucleic acid of the invention against multiple HIV-1 viruses of the same or different subtypes.

Some recombinant gp120 nucleic acid or polypeptide variants of the invention induce the production of a titer of neutralizing antibodies against at least one HIV-1 virus that is at least equal to or greater than the titer of neutralizing antibodies induced against the at least one HIV-1 virus by a WT HIV-1 gp120 polypeptide (e.g., JRCSF gp120 polypeptide). Some such recombinant gp120 nucleic acid or polypeptide variants of the invention induce a higher titer of neutralizing antibodies against at least two HIV-1 viruses of the same or different subtypes than is induced against the at least two HIV-1 viruses by a WT HIV-1 gp120 polypeptide. Antibody production can be determined using known assays (e.g., ELISA assays).

An injectable pharmaceutical composition comprising a suitable, pharmaceutically acceptable carrier (e.g., PBS) and an effective amount of the polypeptide can be delivered intramuscularly, intraperitoneally, subdermally, transdermally, subcutaneously, or intradermally to the host for in vivo. Alternatively, biolistic protein delivery techniques (vaccine gun delivery) can be used (examples of which are discussed elsewhere herein). Any other suitable technique also can be used. Polypeptide administration can be facilitated via liposomes (examples further discussed below).

While the following discussion is primarily directed to nucleic acids, it will be understood that it applies equally to nucleic acid vectors of the invention. A nucleic acid of the invention or composition thereof can be administered to a host by any suitable administration route. In some aspects of the invention, administration of the nucleic acid is parenteral (e.g., subcutaneous, intramuscular, or intradermal), topical, or transdermal. The nucleic acid can be introduced directly into a tissue, such as muscle, by injection using a needle or other similar device. See, e.g., Nabel et al. (1990), supra; Wolff et al. (1990) Science 247:1465-1468), Robbins (1996) Gene Therapy Protocols, Humana Press, NJ, and Joyner (1993) Gene Targeting: A Practical Approach, IRL Press, Oxford, England, and U.S. Pat. Nos. 5,580,859 and 5,589,466. Other methods such as “biolistic” or particle-mediated transformation (see, e.g., U.S. Pat. Nos. 4,945,050 and 5,036,006, Sanford et al., J. Particulate Sci. Tech. 5:27-37 (1987), Yang et al., Proc. Natl. Acad. Sci. USA 87:9568-72 (1990), and Williams et al., Proc. Natl. Acad. Sci. USA 88:2726-30 (1991)). These methods are useful not only for in vivo introduction of DNA into a subject, such as a mammal, but also for ex vivo modification of cells for reintroduction into a mammal (which is discussed further elsewhere herein).

For standard gene gun administration, the vector or nucleic acid of interest is precipitated onto the surface of microscopic metal beads. The microprojectiles are accelerated with a shock wave or expanding helium gas, and penetrate tissues to a depth of several cell layers. For example, the Accel™ Gene Delivery Device manufactured by Agacetus, Inc. Middleton Wis. is suitable for use in this embodiment. The nucleic acid or vector can be administered by such techniques, e.g., intramuscularly, intradermally, subdermally, subcutaneously, and/or intraperitoneally. Additional devices and techniques related to biolistic delivery International Patent Applications WO 99/2796, WO 99/08689, WO 99/04009, and WO 98/10750, and U.S. Pat. Nos. 5,525,510, 5,630,796, 5,865,796, and 6,010,478.

The nucleic acid can be administered in association with a transfection-facilitating agent, examples of which were discussed above. The nucleic acid can be administered topically and/or by liquid particle delivery (in contrast to solid particle biolistic delivery). Examples of such nucleic acid delivery techniques, compositions, and additional constructs that can be suitable as delivery vehicles for the nucleic acids of the invention are provided in, e.g., U.S. Pat. Nos. 5,591,601, 5,593,972, 5,679,647, 5,697,901, 5,698,436, 5,739,118, 5,770,580, 5,792,751, 5,804,566, 5,811,406, 5,817,637, 5,830,876, 5,830,877, 5,846,949, 5,849,719, 5,880,103, 5,922,687, 5,981,505, 6,087,341, 6,107,095, 6,110,898, and International Pat. Appn Publ. Nos. WO 98/06863, WO 98/55495, and WO 99/57275.

The choice of administration/delivery technique and the form of the antigenic polypeptide of the invention, such as a gp120 polypeptide variant antigen (or polynucleotide encoding such antigen), can influence the type of immune response observed upon administration to a subject. For example, gene gun delivery of many antigens is associated with a Th2-biased response (indicated by higher IgG1 antibody titers and comparatively low IgG2a titers). The bias of a particular immune response enables the physician or artisan to direct the immune response promoted by administration of the polypeptide and/or polynucleotide of the invention.

Alternatively, the nucleic acid can be administered to the host by way of liposome-based gene delivery. Exemplary techniques and principles related to liposome-based gene delivery is provided in, e.g., Debs and Zhu (1993) WO 93/24640; Mannino and Gould-Fogerite (1988) BioTechniques 6(7):682-691; Rose U.S. Pat. No. 5,279,833; Brigham (1991) WO 91/06309; Brigham et al. (1989) Am. J. Med. Sci. 298:278-281; Nabel et al. (1990) Science 249:1285-1288; Hazinski et al. (1991) Am. J. Resp. Cell Molec. Biol. 4:206-209; and Wang and Huang (1987) Proc. Natl. Acad. Sci. USA 84:7851-7855), and Felgner et al. (1987) Proc. Natl. Acad. Sci. USA 84:7413-7414). Suitable liposome pharmaceutically acceptable compositions that can be used to deliver the nucleic acid are further described elsewhere herein.

Any immunogenic amount of nucleic acid can be used in the methods of the invention. Typically, where the nucleic acid is administered by injection, about 50 micrograms (μg) to 10 mg, about 1 mg to 8, about 2 mg to about mg, about 100 μg to about 2.5 mg, typically about 500 μg to about 2 mg or about 800 μg to about 1.5 mg, and often about 2 mg or about 1 mg is administered. In one exemplary application, to induce an immune response against HIV virus, e.g., a pharmaceutical comprising PBS and 10 mg of a DNA vector encoding a gp120 polypeptide variant (e.g., SEQ ID NO:1) (or an effective amount thereof) is administered by injection or electroporation or other suitable delivery method (e.g., gene gun, impressing through the skin, and lipofection) to a human subject in need of treatment (e.g., a human desiring to be immunized so as to prevent or inhibit HIV-1 infection). An exemplary vector is shown in FIG. 1. If desired, following administration of one or more such DNA vectors, one or more protein boosts can be administered to the subject at periodic intervals by injection to enhance the immune response; e.g., a composition comprising PBS (or other carrier) and 500 μg of the same gp120 polypeptide variant (e.g., SEQ ID NO:1) of the invention (i.e., homologous protein boost) or a different gp120 polypeptide variant (e.g., SEQ ID NO:2) (i.e., heterologous protein boost) is administered.

The amount of DNA plasmid for use in the methods of the invention where administration is via a gene gun, e.g., is often from about 100 to about 1000 times less than the amount used for direct injection (e.g., via standard needle injection). Despite such sensitivity, at least about 1 μg of the nucleic acid may be used in such biolistic delivery techniques.

RNA or DNA viral vector systems can be useful for delivery nucleic acids encoding gp120 polypeptide variants of the invention. Viral vectors can be administered directly to a subject in vivo or they can be used to treat cells in vitro and the modified cells are administered to the subject in an ex vivo format. Useful viral vectors include those discussed above, such as adeno-associated, adenoviral, retroviral, lentivirus, and herpes simplex virus vectors. With such viral vectors, a nucleic acid of the invention can be readily transferred into target cells and tissues of the subject. Additionally, with the retrovirus, lentivirus, and adeno-associated virus gene transfer methods, a nucleic acid of the invention can be integrated into the host genome may be possible, thereby resulting in continuing expression of the inserted nucleic acid.

Delivery of a viral vector of the invention comprising at least one nucleic acid of the invention to a subject is believed capable of promoting an immune response to at least one HIV virus of at least one subtype in the subject to whom the vector is administered. For example, the viral vector of this invention can comprising a foreign gp120 polypeptide variant-encoding nucleic acid for the expression of a gp120 polypeptide variant effective in inducing an immune response against one or more HIV viruses (e.g., one or more HIV-1 viruses) of one or more HIV subtypes. Optionally, some prophylactic and/or therapeutic methods of the invention, including vaccination and immunization protocols, are practiced with a dosage of a suitable viral vector sufficient to induce a detectable immune response. Any suitable viral vector, in any suitable concentration, can be used to induce the immune response. For example, to the subject host can be administered a population of retroviral vectors (examples of which are described in, e.g., Buchscher et al. (1992) J. Virol. 66(5) 2731-2739, Johann et al. (1992) J. Virol. 66 (5):1635-1640 (1992), Sommerfelt et al., (1990) Virol. 176:58-59, Wilson et al. (1989) J. Virol. 63:2374-2378, Miller et al., J. Virol. 65:2220-2224 (1991), Wong-Staal et al., PCT/US94/05700, Rosenburg and Fauci (1993) in FUNDAMENTAL IMMUNOLOGY, THIRD EDITION Paul (ed.) Raven Press, Ltd., New York and the references therein), an AAV vector (as described in, e.g., West et al. (1987) Virology 160:38-47, Kotin (1994) Human Gene Therapy 5:793-801, Muzyczka (1994) J. Clin. Invest. 94:1351, Tratschin et al. (1985) Mol. Cell. Biol. 5(11):3251-3260, U.S. Pat. Nos. 4,797,368 and 5,173,414, and Int'l Patent Appn Publ. No. WO 93/24641), or an adenoviral vector (as described in, e.g., Berns et al. (1995) Ann. NY Acad. Sci. 772:95-104; Ali et al. (1994) Gene Ther. 1:367-384; and Haddada et al. (1995) Curr. Top. Microbiol. Immunol. 199 (Pt 3):297-306), such that immunogenic levels of expression of the nucleic acid included in the vector thereby occurs in vivo resulting in the desired immune response. Other suitable types of viral vectors are described elsewhere herein (including alternative examples of suitable retroviral, AAV, and adenoviral vectors).

Suitable infection conditions for these and other types of viral vector particles are described in, e.g., Bachrach et al., J. Virol., 74(18), 8480-6 (2000), Mackay et al., J. Virol., 19(2), 620-36 (1976), and FIELDS VIROLOGY, supra. Additional techniques useful in the production and application of viral vectors are provided in, e.g., “Practical Molecular Virology: Viral Vectors for Gene Expression” in METHODS IN MOLECULAR BIOLOGY, vol. 8, Collins, M. Ed., (Humana Press 1991), VIRAL VECTORS: BASIC SCIENCE AND GENE THERAPY, 1^{st} Ed. (Cid-Arregui et al., Eds.) (Eaton Publishing 2000), “Viral Expression Vectors,” in CURRENT TOPICS IN MICROBIOLOGY AND IMMUNOLOGY, Oldstone et al., Eds. (Springer-Verlag, NY, 1992), and “Viral Vectors” in CURRENT COMMUNICATIONS IN BIOTECHNOLOGY, Gluzman and Hughes, eds. (Cold Spring Harbor Laboratory Press, 1988).

The toxicity and therapeutic efficacy of vectors or viruses that include one or more molecules of the invention are determined using standard pharmaceutical procedures in cell cultures or experimental animals. One can determine the MLD₅₀ (the minimum dose lethal to 50% of the population) and/or the ED₅₀ (the dose therapeutically effective in 50% of the population) using procedures presented herein and those otherwise known in the art. See also S. Plotkin and W. Orenstein, VACClNES (W. B. Saunders Co. 1999 3d ed.) for suggested doses for known viral vaccines. Nucleic acids, polypeptides, proteins, fusion proteins, transduced cells and other formulations of the present invention can be administered in an amount determined, e.g., by the MLD₅₀ of the formulation, and the side-effects thereof at various concentrations, as applied to the mass and overall health of the patient. Thus, for example, the invention provides a method of inducing an immune response by administering a dose equal or greater to the ED₅₀ of a pharmaceutically acceptable composition comprising a population of virus-like particles or viruses (e.g., attenuated or replication-deficient virus) that comprises a polypeptide or nucleic acid of the invention. Administration can be accomplished via single dose or divided doses (either by co-administration, serial administration, or combinations thereof). Administration techniques and protocols are described in, e.g., Plotkin (VAcciNEs) supra and other references cited herein. In a related sense, techniques for assessing dosage of the nucleic acid, polypeptide, vector, and cell compositions effective for inducing immunity are described in, e.g., European Patent Appn No. 1 156 333 and references cited therein.

The viral vector can be targeted to particular tissues, cells, and/or organs of a subject, e.g., mammal. Examples of such vectors are described above. For example, the viral vector or nucleic acid vector can be used to selectively deliver the nucleic acid sequence of the invention to monocytes, dendritic cells, cells associated with dendritic cells (e.g., keratinocytes associated with Langerhans cells), T-cells, and/or B-cells. The viral vector can be a replication-deficient viral vector. The viral vector particle also can be modified to reduce host immune response to the viral vector, thereby achieving persistent gene expression. Such “stealth” vectors are described in, e.g., Martin, Exp. Mol. Pathol. 66(1):3-7 (1999), Croyle et al., J. Virol. 75(10):4792-801 (2001), Rollins et al., Hum. Gene Ther. 7(5):619-26 (1996), Ikeda et al., J. Virol. 74(10):4765-75 (2000), Halbert et al., J. Virol. 74(3):1524-32 (2000), and Int'l Patent Appn Publ. No. WO 98/40509. Alternatively or additionally, the viral vector particles can be administered by a strategy selected to reduce host immune response to the vector particles. Strategies for reducing immune response to the viral vector particle upon administration to a host are provided in, e.g., Maione et al., Proc. Natl. Acad. Sci. USA 98(11):5986-91 (2001), Morral et al., Proc. Natl. Acad. Sci. USA 96(22):2816-21 (1999), Pastore et al., Hum. Gene Ther. 10(11):1773-81 (1999), Morsy et al., Proc. Natl. Acad. Sci. USA 95(14):7866-71 (1998), Joos et al., Hum.

Gene Ther. 7(13):1555-66 (1996), Kass-Eisler et al., Gene Ther. 3(2):154-62 (1996), U.S. Pat. Nos. 6,093,699, 6,211,160, 6,225,113, U.S. Patent Application Publ. No. 2001-0066947A1.

Any suitable population and concentration (dosage) of viral vector particles can be used to induce the immune response in the subject host. In some aspects of the invention, at least about 1×10² particles are typically used (e.g., the method can comprises administering a composition comprising at least from about 1×10² particles/ml to about 1×10¹¹ particles/ml of a suitable viral vector particle in about 1-2 milliliters (m1) injectable and pharmaceutically acceptable solution). When delivered to a host, the population of viral vector particles is such that the multiplicity of infection (MOI) is at least from about 1 to about 100, including from at least about 5 to about 30. Considerations in viral vector particle dosing are described elsewhere herein.

The term “prime” generally refers to the administration or delivery of a polypeptide of the invention, a nucleic acid of the invention encoding such polypeptide, or a virus or VLP comprising such a polypeptide or nucleic acid in vitro to a cell culture or population of cells, or in vivo to a subject, or ex vivo to a tissue or cells of a subject. The first administration or delivery (primary contact) may not be sufficient to induce or promote a measurable immune response (e.g., antibody response), but may be sufficient to induce a memory response or an enhanced secondary response. The term “challenge” generally refers to any procedure that induces, promotes, or modulates an immune response.

The initial delivery or administration of a polypeptide, nucleic acid, virus, or VLP of the invention is followed by one or more secondary (repeated) administrations of the polypeptide or nucleic acid. For example, initial administration of a polypeptide can be followed with a repeat administration (“prime boost”) of a substantially similar or identical dose of the polypeptide (e.g., about 5 μg to about 1 mg, or about 5 μg to 0.1 mg of polypeptide in a 1-2 ml injectable and pharmaceutically acceptable solution). Typically, the prime boost is administered at least 7 days after the initial polypeptide administration. The prime boost may be administered about 14-35 days or about 2, 4, 6, 12, or 24 months after initial polypeptide administration.

If desired, a second repeat administration (or “secondary boost”) is performed with a substantially similar or identical dose of the polypeptide at about 2-9, 3-6 months, 9-18 months, or about 12 or 24 months after the initial polypeptide administration. A similar procedure can be used to administer a nucleic acid, virus or VLP of the invention. Additional administration strategies and doses are discussed throughout.

Alternatively, a different nucleic acid, polypeptide, vector, cell, or antibody of the invention is used to boost the immune response induced by the first dosage of a nucleic acid, polypeptide, vector, cell, or antibody of the invention. For example, administration to a subject of an initial dosage of a composition comprising a polypeptide comprising the polypeptide sequence of SEQ ID NO:1 or SEQ ID NO:2, or other suitable antigenic or immunogenic polypeptide of the invention, can be followed by administration to the subject of an immunogenic second dose of a pox virus, such as a vaccinia virus, canary pox virus, or MVA viral vector, which second dose can further be followed by a third, fourth, or even fifth boost of such a pox virus, wherein such further doses of pox virus enhance the immune response against HIV induced by the initial dose of the immunogenic polypeptide of the invention.

The following strategies, summarized in Table 3, provide additional and particular examples of such prime-boosting administration regimens. These strategies are particular examples and do not in any way restrict the ability to use other prime-boosting or different administration strategies, examples of which are provided elsewhere herein.

TABLE 3
Exemplary Prime-Boost Administration Strategies

| 1^{st} Administration | | | |
|---|---|---|---|
| (Prime) | Boost 1 | Boost 2 | Boost 3 |
| DNA | DNA injection (i.m.) | DNA | Adenovirus (Ad) injection |
| injection | | injection | (i.m.) (e.g., injection with |
| (i.m.) | | (i.m.) | about 1 × 10⁹-1 × 10¹¹ |
| | | | plaque forming units (PFU) |
| | | | adenoviral (Ad) vector |
| | | | comprising a heterologous |
| | | | or homologous protein or |
| | | | comprising a nucleic acid |
| | | | encoding a heterologous or |
| | | | homologous protein for the |
| | | | boost)* (e.g., about 1 × 10⁹- |
| | | | 1 × 10¹¹ PFU Ad vector |
| | | | comprising a nucleic acid |
| | | | that encodes the polypeptide |
| | | | sequence of SEQ ID NO: 1) |
| DNA | Pox virus (e.g., MVA, canary | Repeat | |
| injection | pox, avipox, or ALVAC) boost | boost 1 if | |
| (i.m.) | (i.m.) (virus comprises a | desired | |
| | heterologous or homologous | | |
| | protein or comprises a nucleic | | |
| | acid encoding a heterologous | | |
| | or homologous protein for the | | |
| | boost)* (e.g., about 2 × 10⁷ | | |
| | PFU canary pox vector | | |
| | comprising a nucleic acid that | | |
| | encodes the polypeptide | | |
| | sequence of SEQ ID NO: 1) | | |
| Adenovirus | DNA boost (i.n.) | | |
| (i.n.) | | | |
| DNA | Protein boost* | | |
| injection | (i.m., i.d., i.n., or s.c.) | | |
| (i.m., i.d., | | | |
| i.n., or s.c.) | | | |
| DNA | Protein boost* | Protein | Protein boost* |
| injection | (i.m., i.d., i.n., or s.c.) | boost* | (i.m., i.n., i.d., or s.c.) |
| (i.m., i.d., | | (i.m., i.d., | |
| i.n., or s.c.) | | i.n., or | |
| | | s.c.) | |
| Protein | Protein boost* | | |
| prime (s.c. | (i.m. or s.c.) | | |
| or i.m.) | | | |
| DNA | DNA boost | Protein | |
| prime | | boost* | |
| DNA | Protein boost* | | |
| prime | | | |
| DNA | Adenovirus boost (comprising | | |
| prime | a heterologous or homologous | | |
| | protein for the boost or | | |
| | comprising a nucleic acid | | |
| | encoding a heterologous or | | |
| | homologous protein)* | | |
| Liposome- | Protein boost* | | |
| associated | | | |
| Nucleic | | | |
| acid vector | | | |
| prime | | | |
| DNA | DNA | DNA | Protein boost* |
| (i.m. - e.g., | (i.m. - e.g., 1 to 10 mg | (i.m. - | (0.1 to 0.5 mg) |
| 1 mg pMAmp) | pMAmp) | e.g., 1 to | |
| | | 10 mg | |
| | | pMAmp) | |
| DNA(i.d. - e.g., | DNA | DNA | Protein boost* |
| 1 mg | (i.d. - e.g., 1 to 10 mg | (i.d. - | (0.1 to 0.5 mg) |
| pMAmp) | pMAmp) | e.g., 1 to | |
| | | 10 mg | |
| | | pMAmp) | |

i.m.: intramuscular
i.d.: intradermal
i.n.: intranasal
s.c.: subcutaneous
*A protein boost may comprise a heterologous or homologous protein. A heterologous protein boost typically comprises a protein comprising a polypeptide sequence that differs from the polypeptide sequence of the protein encoded by the nucleic acid (e.g., DNA) used for the prime immunization (e.g., DNA prime or vector prime). A homologous protein boost typically comprises a protein comprising a polypeptide sequence that is identical or substantially identical to the polypeptide sequence of the protein encoded by the nucleic acid (e.g., DNA) used for the prime immunization.

The term “DNA injection” in Table 3 refers to injection of a nucleic acid or nucleic acid vector of the invention. For example, a DNA injection can include injection of a monocistronic pMAmp expression vector encoding SEQ ID NO:1 or bicistronic pMAmp vector comprising a sequence encoding SEQ ID NO:1 and a second sequence encoding an immunostimulatory/anti-tumor cytokine (e.g., GM-CSF or TNF-α) or a costimulatory polypeptide (e.g., B7-1 or B7-2). A heterologous protein boost in Table 3 refers to the administration of a second polypeptide of the invention that differs from the polypeptide(s) of the invention administered in the prime administration or expressed by the DNA, plasmid, or viral vector in the prime administration. Routes of administration (e.g., s.c. (subcutaneous)) provided in Table 3 are exemplary only—any suitable route of administration can be used for these or any other prime-boosting strategy described herein. The type of administration strategy can influence the type of immune response. For example, administration of a recombinant adenovirus is expected to provide effective antibody production, whereas administration of a DNA vector (e.g., a pMAmp vector) followed by a protein, DNA, and/or viral vector boost is expected to provide effective T cell responses.

Adjuvants

Adjuvant may be useful in augmenting an immune response induced in a subject by the adminisatration of a nucleic acid or polypeptide of the invention. An adjuvant may increase the subject's immune system response to such nucleic acid or polypeptide. One or more adjuvants may be administered simultaneously or sequentially in any immunization or immune-stimulating method described herein. Thus, for example, any method comprising administering to a subject a polypeptide or nucleic acid of the invention described herein can also include the co-administration to the subject of one or more suitable adjuvants. For example, priming by immunization of a subject with nucleic acid encoding an immunogen or antigen of the invention (e.g., DNA encoding a specific gp120 polypeptide variant) followed by administration to the subject of a recombinant immunogenic or antigenic protein of the invention (e.g., the same gp120 polypeptide variant (i.e., homologous protein) or a different gp120 polypeptide variant (i.e., heterologous protein)) is one representative method for achieving sufficient antibody titers and/or broadening the immune response. Alternatively, a subject may simply be immunized with nucleic acid encoding an immunogen or antigen of the invention to induce an immune response. Or, a recombinant immunogenic or antigenic protein of interest may be administered to a subject in an amount effective to induce an immune response. In each such method, if desired, one or more adjuvants may also be administered to the subject simultaneously or sequentially with the nucleic acid or polypeptide. Exemplary amounts of nucleic acid or polypeptide typically administered in such immunization protocols is described elsewhere. The amount of adjuvant administered may depend on the adjuvant and may be the amount of the adjuvant utilized in commercially available vaccines or clinical therapeutic or prophylactic treatment regimens. Typically, a gp120 immunogen is adjuvanted (mixed) with an amount of the adjuvant that is sufficient to boost the immune response (e.g., antibody titer) beyond the immune response induced by the gp120 immunogen alone. The amount of adjuvant used in the immunization protocols involving rabbits, which are discussed in the Examples below, may be scaled up appropriately for a larger mammal, such as human, based upon the mammal's weight. In one exemplary aspect, a recombinant gp120 polypeptide variant of the invention is co-administered to a subject with a suitable amount of an adjuvant (e.g., alum, MPL+alum, etc.) sufficient to augment the immune response (e.g., antibody titers) induced in the subject by the gp120 polypeptide variant alone.

Numerous types of adjuvants that can be suitable for co-administration or serial administration with one or more polypeptides or nucleic acids of the invention are known in the art. Adjuvants can be selected based on their ability to augment or enhance the immune response induced by a particular immunization method (e.g., DNA prime only; DNA prime/protein boost; protein administration only). Examples of suitable adjuvants include Freund's emulsified oil adjuvants (complete and incomplete), inorganic adjuvants, such as, e.g., alum (aluminum hydroxide and/or aluminum phosphate), lipopolysaccharides (e.g., bacterial LPS), liposomes (including dried liposomes and cytokine-containing (e.g., IFN-γ-containing and/or GM-CSF-containing) liposomes), endotoxins, calcium phosphate and calcium compound microparticles (see, e.g., Int'l Patent Appn Publ. No. WO 00/46147), mycobacterial adjuvants, Arlacel A, mineral oil, emulsified peanut oil adjuvant (adjuvant 65), Bordetella pertussis products/toxins, Cholera toxins, non-ionic block polymer surfactants, Corynebacterium granulosum derived P40 component, fatty acids, aliphatic amines, paraffinic and vegetable oils, beryllium, and immunostimulating complexes (ISCOMs—reviewed in, e.g., Hoglund et al. “ISCOMs and immuno stimulation with viral antigens” in SUBCELLULAR BIOCHEMISTRY (Ed. Harris, J. R.) Plenum, New York, 1989, pp. 39-68), Morein et al., “The ISCOM—an approach to subunit vaccines” in RECOMBINANT DNA VACClNES: RATIONALE AND STRATEGY (Ed. Isaacson, R. E.) Marcel Dekker, New York, 1992, pp. 369-386, and Morein et al., Clin. Immunotherapeutics 3:461-75 (1995)). Recently, monophosphoryl lipid A, ISCOMs with Quil-A, and Syntex adjuvant formulations (SAFs) containing the threonyl derivative or muramyl dipeptide also have been under consideration for use in human vaccines.

Additional examples of suitable adjuvants are described in, e.g., Vogel et al., A COMPENDIUM OF VACCINE ADJUVANTS AND EXCIPIENTS (2d Ed) (see world wide website address niaid.nih.gov/aidsvaccine/pdf/compendium.pdf), Bennet et al., J. Immun. Meth. 153:31-40 (1992), Bessler et al., Res. Immunol. 143(5):519-25 (1992), Woodard, Lab. Animal Sci. 39(3):222-5 (1989), Vogel, AIDS Res. and Human Retroviruses 11(10):1277 1278 (1995), Leenaars et al., Vet. Immunol. Immunopath. 40:225-241 (1995), Linblak et al., Scandinavian J. Lab. Animal Sci. 14:1-13 (1987), Buiting et al., Res. Immunol. 143(5):541-548 (1992), Gupta and Siber, Vaccine (14):1263-1276 (1996), and U.S. Pat. Nos. 6,340,464, 6,328,965, 6,299,884, 6,083,505, 6,080,725, 6,060,068, 5,961,970, 5,814,321, 5,747,024, 5,690,942, 5,679,356, 5,650,155, 5,585,099, 4,395,394, and 4,370,265.

Table 4 presents additional exemplary adjuvants that may be utilized in the immunization methods of the invention, including the prophylactic and therapeutic methods discussed elsewhere herein.

TABLE 4
Exemplary Adjuvants for Administration with gp120 Immunogens

| Adjuvant | Source | Comments | Reference |
|---|---|---|---|
| Monophosphoryl | Corixa Corp. | MPL + alum | Thoelen et al., Vaccine |
| lipid A (MPL) | | | 19: 2400-2403 (2001) |
| MF59 | Chiron Corp. | Used in an approved | Singh et al., Nat. |
| | | product in Europe | Biotechnol. 17: 1075-1081 |
| | | | (1999) |
| CpG | Coley Pharma. | Useful to stimulate | Krieg et al., Annu. Rev. |
| Oligonucleotides | | innate immunity and | Immunol. 20: 709-760 |
| | | TH1-like responses | (2002) |
| QS21 | Antigenics, Inc. | Has been tested in | Singh et al., Nat. |
| | | several clinical trials | Biotechnol. 17: 1075-1081 |
| | | | (1999) |
| Montanide ISA | Seppic | Commercially | Toledo et al. , Vaccine |
| 720 | | available | 19: 4328-4336 (2001) |
| Ribi Adjuvant | Corixa Corp. | RAS is | Bennett et al., J. Immunol. |
| System (RAS) | | recommended for | Methods 153: 31-40 (1992) |
| | | use in rabbits (e.g., | |
| | | for comparative | |
| | | purposes) | |
| AS02* | GlaxoSmithKline | AS02 includes | Garcon et al., Expert Rev. |
| (*formerly | | monophosphoryl | Vaccines 2(2): 231-8 (2003); |
| known as | | lipid A (MPL), a | Gerard et al., Vaccine |
| SBAS 2) | | bacterial cell wall | 19: 2583-2589 (2001) (see |
| | | component with | also SBAS 2 described |
| | | adjuvant activity, | therein) |
| | | and QS21 | |

An exemplary protocol for testing an adjuvant comprises two different plasmids encoding two gp120 polypeptide variants, respectively, wherein each plasmid is administered to a rabbit (or other host) host by three monthly injections of DNA by electroporation up to 2 months into groups of 30 rabbits per plasmid. For each plasmid, the homologous recombinant protein suitably formulated in each of the chosen adjuvants is then injected at 3, 4, 5, and 6 months into six rabbits. For exemplary suitable amount of adjuvant to be used, see the Examples below. Test bleeds are withdrawn two weeks after each injection beginning at 2.5 months. IgG is purified from each serum sample and submitted to the viral neutralization assay discussed in detail elsewhere herein. From such experiments, an adjuvant gives the highest, most persistent and/or broadest neutralization activity, as well as the number of preferred protein immunizations, can be determined.

Administration Formats

As indicated above, administration of a nucleic acid of the invention also may be followed by boosting (at least a prime, or at least a prime and secondary boost). A “prime” is typically the first immunization. An initial nucleic acid administration can be followed by a repeat administration of the nucleic acid at least about 7 days, about 14-35 days, or about 2, 4, 6, 9, or 12 months, after the initial nucleic acid administration. The amount administered in the repeat administration is typically substantially similar (if not identical) to the dose of the nucleic acid initially administered, (e.g., about 50 μg to about 15 or 20 mg, or 1 mg to about 10 mg, or 2-5 mg in a 1-2 ml volume injectable and pharmaceutically acceptable solution).

Alternatively, the initial administration of the nucleic acid can be followed by a prime boost of an immunogenic or antigenic amount of polypeptide at such a time. A secondary boost also may be performed with nucleic acid and/or polypeptide, in an amount similar to that used in the primary boost and/or the initial nucleic acid administration, at about 2-9, 3-6 months, 9-18 months, or about 12 or 24 months after the initial polypeptide administration. Any number of boosting administrations of nucleic acid and/or polypeptide can be performed.

The polypeptide, nucleic acid, and/or vector of the invention can be used to promote any suitable immune response to at least HIV-1 virus of one or more subtypes in any suitable context. For example, at least one polypeptide, nucleic acid, and/or vector of the invention can be administered as a prophylactic in an immunogenic or antigenic amount to a mammal (e.g., a human) that has not been infected with an HIV-1 virus. The administration of the at least one polypeptide, nucleic acid, and/or vector may be in an amount effective to induce in the subject to whom it is administered a detectable immune response that provides partial or inhibitory immune response against a challenge with at least one HIV-1 of at least one subtype, and, as such, can be considered a “vaccine” against infection by that HIV-1 virus. The administration of an effective amount of at least one polypeptide, nucleic acid, and/or vector of the invention may induce in the subject a protective immune response against challenge with two or more HIV-1 viruses and, as such, can be considered a “vaccine” against infection by those viruses. The polypeptide, nucleic acid, and/or vector may be administered to the subject in an amount effective to induce a protective immune response in a human at risk for HIV-1 infection.

At least one polypeptide, polynucleotide, and/or vector of the invention may be administered to a mammal (e.g., a human) that has been previously infected with at least one HIV-1 virus of at least one particular subtype, in an effective amount such that the infection is decreased and/or inhibited. The polypeptide, nucleic acid, and/or vector may be administered to the subject in an amount effective to induce an immune response that effectively reduces or inhibits the initial dose (inoculum) of the virus.

The polynucleotides and vectors of the invention can be delivered by ex vivo delivery of cells, tissues, or organs. As such, the invention provides a method of promoting an immune response to an HIV-1 virus comprising inserting at least one nucleic acid of the invention and/or a vector of the invention into a population of cells and implanting the cells in a mammal. Ex vivo administration strategies are known in the art (see, e.g., U.S. Pat. No. 5,399,346 and Crystal et al., Cancer Chemother. Pharmacol. 43(Suppl.):S90-S99 (1999)). Cells or tissues can be injected by electroporation or by a needle or gene gun or implanted into a mammal ex vivo. Briefly, in ex vivo techniques, a culture of cell (e.g., organ cells, cells of the skin, muscle, etc.) or target tissue is provided, or removed from the host, contacted with the vector or polynucleotide composition, and then reimplanted into the host (e.g., using techniques described in or similar to those provided in). Ex vivo administration of the nucleic acid can be used to avoid undesired integration of the nucleic acid and to provide targeted delivery of the nucleic acid or vector. Such techniques can be performed with cultured tissues or synthetically generated tissue. Alternatively, cells can be provided or removed from the host, contacted (e.g., incubated with) an amount of a polypeptide of the invention that is effective in prophylactically inducing an immune response to an HIV-1 virus (e.g., a protective immune response, such as a protective neutralizing antibody response, against an HIV-1 virus) when the cells are implanted or reimplanted to the host. The contacted cells are then delivered or returned to the subject to the site from which they were obtained or to another site (e.g., including those defined above) of interest in the subject to be treated. If desired, the contacted cells may be grafted onto a tissue, organ, or system site (including all described above) of interest in the subject using standard and well-known grafting techniques or, e.g., delivered to the blood or lymph system using standard delivery or transfusion techniques. Such techniques can be performed with any suitable type of cells. For example, in one aspect, activated T cells can be provided by obtaining T cells from a subject (e.g., mammal, such as a human) and administering to the T cells an amount of one or more polypeptides of the invention effective to activate the T cells (or administering an effective amount of one or more nucleic acids of the invention with a promoter such that uptake of the nucleic acid into one or more such T cells occurs and sufficient expression of the nucleic acid results to produce an amount of a polypeptide effective to activate said T cells). The activated T cells are then returned to the subject. T cells can be obtained or isolated from the subject by a variety of methods known in the art, including, e.g., by deriving T cells from peripheral blood of the subject or obtaining T cells directly from a tumor of the subject. Other cells for ex vivo methods include explanted lymphocytes, particularly B cells, antigen presenting cells (APCs), such as dendritic cells, and more particularly Langerhans cells, monocytes, macrophages, bone marrow aspirates, or universal donor stem cells. A preferred aspect of ex vivo administration of a polynucleotide or polynucleotide vector can be the assurance that the polynucleotide has not integrated into the genome of the cells before delivery or readministration of the cells to a host. If desired, cells can be selected for those where uptake of the polynucleotide or vector, without integration, has occurred, using standard techniques known in the art.

The invention includes a method of inducing an immune response in a subject to at least one HIV-1 virus of at least one subtype comprising: (a) providing a population of B cells, dendritic cells, or both; (b) transforming the cells with at least one nucleic acid of the invention such that the nucleic acid does not integrate into a genome of any of the cells, and (c) delivering an effective amount of the cells to the subject, wherein the cells express the at least one nucleic acid after delivery and induce an immune response to the at least one HIV-1 virus in the subject. In some such methods, prior to transforming the cells with the nucleic acid, the cells are obtained from a subject, and after transformation with the at least one nucleic acid, the cells are delivered to the same subject.

In another aspect, the invention provides a method of inducing an immune response by administering an effective amount of a population of recombinant VLPs or attenuated viruses of the invention, formed by populations of polypeptides of the invention. The administration of VLPs or attenuated viruses is carried out using techniques similar to those used for the administration of polypeptides and viral vectors as described above. VLPs can be administered in a pharmaceutically acceptable injectable solution into or through the skin, intramuscularly, or intraperitoneally. The skin and muscle are generally preferred targets for administration of the polypeptides, vectors, and polynucleotides of the invention, by any suitable technique. Thus, the delivery of the polypeptide, polynucleotide, or vector of the invention into or through the skin of a subject (e.g., mammal), is a feature of the invention. Such administration can be accomplished by transdermal devices, or, more typically, biolistic delivery of the polypeptide, polynucleotide, and/or vector to, into, or through the skin of the mammal, or into exposed muscle of the subject. Transdermal devices provided by the invention, described elsewhere herein, for example, can be applied to the skin of a host for a suitable period such that sufficient transfer of a polynucleotide and/or vector to the mammal occurs, thereby promoting an immune response to at least one HIV virus. Muscular administration is more typically facilitated by injection of a liquid solution comprising a polypeptide, polynucleotide, or vector of the invention. Particular cells that can be targeted include dendritic cells, other APCs, B cells, monocytes, T cells (including T helper cells), and cells associated with such immune system cells (e.g., keratinocytes or other skin cells associated with Langerhans cells). Targeting of vectors and nucleic acids of the invention is described elsewhere herein. Such targeted administration can be performed with nucleic acids or vectors comprising nucleic acids operably linked to cell and/or tissue-specific promoters, examples of which are known in the art.

The polynucleotide of the invention can be administered by any suitable delivery system, such that expression of a recombinant polypeptide occurs in the host resulting in an immune response to at least one HIV-1 virus. For example, an effective amount of a population of bacterial cells comprising a nucleic acid of the invention can be administered to a subject, resulting in expression of a recombinant polypeptide of the invention, and induction of an immune response to HIV viruses in the subject, e.g., mammal. Bacterial cells developed for mammalian gene delivery are known in the art.

Administration of a polynucleotide or vector of the invention is facilitated by application of electroporation to an effective number of cells or an effective tissue target, such that the nucleic acid and/or vector is taken up by the cells, and expressed therein, resulting in production of a recombinant polypeptide of the invention therein and subsequent induction of an immune response to one or more HIV-1 viruses in the mammal.

The nucleic acid, polypeptide, and/or vector of the invention may be co-administered with an effective amount of an additional nucleic acid or additional nucleic vector comprising an additional nucleic acid that increases the immune response to an HIV virus upon administration of the nucleic acid, polypeptide, and/or vector of the invention. Such a second nucleic acid may comprise a sequence encoding a GM-CSF, an interferon (e.g., IFN-gamma) or both, examples of which are discussed elsewhere herein. Alternatively, the second nucleic acid can comprise immunostimulatory (CpG) sequences, as described elsewhere herein. GM-CSF, IFN-gamma, or other polypeptide adjuvants also can be co-administered with the polypeptide, polynucleotide, and/or vector. Co-administration in this respect encompasses administration before, simultaneously with, or after, the administration of the polynucleotide, polypeptide, and/or vector of the invention, at any suitable time resulting in an enhancement of an immune response to an HIV virus, including HIV-1.

In another aspect, the invention provides a method of generating a cytotoxic T cell response in a subject, such as a mammal (e.g., human). The method comprises administering to the subject a nucleic acid comprising a nucleotide sequence encoding at least one polypeptide of the invention, or a vector comprising a nucleotide sequence encoding at least one polypeptide of the invention, in an amount effective to induce a detectable cytotoxic T cell response in the subject, wherein a detectable cytotoxic T cell response is generated. The nucleotide sequence is typically under the control of a promoter that is capable of expressing the polypeptide in the host. The vector may comprise any suitable vector as described elsewhere herein, such as a plasmid vector, viral vector, bacterial vector, yeast vector or plant vector.

Also provided by the invention is a method of generating a cytotoxic T cell response in a subject, such as a mammal (e.g., human), which comprises administering to the subject at least one polypeptide of the invention in an amount effective to induce a detectable cytotoxic T cell response in the subject, wherein a detectable cytotoxic T cell response is generated.

Diagnostic Applications

The invention also provides a diagnostic assay for detecting anti-HIV-1 antibodies. Some polypeptides of the invention are recognized one or more antibodies against one or more HIV-1 viruses. Such polypeptides are recognized by type-specific antisera. These polypeptides of the invention are useful as diagnostic tools to capture antibodies against one or more HIV-1 viruses. Such polypeptides can be used to detect serum antibodies against any of HIV-1 in a biological sample obtained from a mammal, such as a human.

In one aspect, the invention provides a diagnostic method of screening a composition, including as a biological sample obtained from a subject, such as blood or serum, for the presence or absence of one or more anti-HIV antibodies of one or more subtypes by contacting the biological sample with a polypeptide of the invention that is conjugated to a detectable labels. A variety of labels known in the art can be used, and methods for conjugation are also well known. In one aspect, the invention provides a method that comprises contacting a biological sample obtained from a subject (e.g., human), such as blood or serum, with a labeled polypeptide of the invention under conditions such if the sample comprises anti-HIV antibodies (e.g., anti-HIV-1 antibodies), such antibodies bind to the polypeptide to form a mixed composition. The mixed composition is then contacted with at least one affinity-molecule that binds to an anti-HIV antibody. Unbound affinity-molecule is then removed from the mixed composition, and the presence or absence of affinity molecules in the composition is detected, wherein the presence of an affinity molecule is indicative of the presence of anti-HIV antibodies in the sample. The presence of anti-HIV antibodies indicates the subject has been exposed to or infected with HIV (e.g., HIV-1). Any suitable biological sample (i.e., that includes a sufficient quantity of antibodies for analysis, if present) can be used. Serum from a mammal can be obtained and used for such analysis. Alternatively, tissues where antibody concentrations are expected to be high, such as lymphoid tissues, can be analyzed.

The invention also includes an immunoassay for at least one anti-HIV antibody (e.g., HIV-1 antibody), which comprises the use of a polypeptide of the invention as a test sample. The above-described methods can further be modified to form any suitable type of immunoassay, examples of which are described above. Preferred immunoassays include dot-blot assays, ELISA assays (e.g., competitive ELISA assays), and dipstick EIAs. In preparation of such assays, a polypeptide of the invention (e.g., gp120 polypeptide variant) is bound to or associated with a solid or semisolid matrix, to promote antigen-antibody complex formation. The detection of such antibody-antigen complexes is typically facilitated with a reagent suitable for visualization, such as dyes used in ELISA and FACS assays described elsewhere herein. Compositions comprising such elements are provided by the invention. For example, the invention provides a composition comprising at least one polypeptide of the invention bound to a solid matrix, and optionally including a reagent for visualizing an antibody bound to the polypeptide.

The invention also includes a kit for performing such an immunoassay comprising a composition of a polypeptide of the invention, bound to a solid matrix, in combination with a reagent suitable for visualization of antigen-antibody complexes after incubation of the matrix with a biological sample suspected of comprising anti-HIV antibodies (e.g., HIV-1 antibodies).

A suitable substrate for performing an immunoassay to detect one or more anti-HIV virus antibodies in a sample composition is provided by obtaining cell free medium, aspirated from a culture of cells transformed with a polynucleotide of the invention (including a nucleic acid vector), or infected with a viral vector of the invention, which cells at least partially secrete a polypeptide of the invention into the cell medium such that the aspirated medium (supernatant) comprises a sufficient amount of polypeptide for use in the immunoassay. In most instances, as little as about 10 μl of such a cell supernatant can be used as a substrate for a sensitive immunoassay, which is able to detect the presence of antibodies to HIV in a sample of serum obtained from a mammalian host (e.g., a human). Also contemplated is the use of larger amounts of such supernatant (e.g., about 20 μA about 50 μl, about 100 μl, or more), as well as the use of cell lysates of cells transfected with nucleic acids (or nucleic acid vectors) of the invention, as well as of cells infected with viral vectors of the invention. The supernatant can be associated with a matrix for performing EIAs (e.g., with an ELISA plate for ELISA assay or with a suitable membrane for dot-blot assay) or can be directly used in an immunoprecipitation or other direct detection immunohistochemical technique. Similar techniques that can be modified with reference to the polypeptides of the invention are described in, e.g., U.S. Pat. No. 5,939,254 and other references cited herein.

The invention also includes a method of identifying the presence of antibodies to an HIV virus (e.g., HIV-1 virus) in a biological sample obtained from a subject, such as a mammal, comprising contacting at least one polypeptide of the invention (or composition comprising at least one such polypeptide and a carrier or a solid matrix) with a biological sample obtained from the subject under conditions such that an antibody capable of binding to a flavivirus in the biological sample binds to the polypeptide and forms an antibody-polypeptide complex; and detecting the presence of the antibody-polypeptide complex in the biological sample, thereby indicating the presence of antibodies in the biological sample (e.g., blood or serum).

Pools or libraries of two or more polypeptides of the invention also can be used in diagnosis techniques. Alternatively, a polypeptide of the invention can be added to a pool of other molecules (e.g., a pool of polypeptides, such as a collection of viral antigens). Thus, a library comprising two or more polypeptides of the invention is a feature of the invention. Another feature of the invention is a library of polypeptides of the invention (e.g., a collection of fragments of polypeptides of the invention or a collection of substantially identical polypeptides of the invention). The polypeptide(s) of the invention can be used in such libraries for diagnostic techniques (e.g., multiple diagnostic techniques for viral infection and/or other disease diagnosis). For example, a library of pathogenic antigens from pathogens associated with fever (or other disease states), comprising at least one polypeptide of the invention, can be used to diagnose infection of a mammal (e.g., human) by reaction of a biological sample obtained from the mammal with such a library in a manner that a detectable biological reaction between the sample and at least one component of the library will occur, thereby indicating what type of infection the mammal suffers from. The incorporation of one or more polypeptides of the invention in diagnostic chips (“protein chips”) for such diagnostic techniques is a feature of the invention.

Production and Purification Methods

The invention further provides methods of making and purifying the polypeptides, nucleic acids, vectors, viruses, pseudoviruses VLPs, and cells of the invention. In one aspect, the invention provides a method of making a recombinant polypeptide of the invention by introducing a nucleic acid of the invention into a population of cells in a culture medium, culturing the cells in the medium (for a time and under conditions suitable for desired level of gene expression) to produce the polypeptide, and isolating the polypeptide from the cells, culture medium, or both. The nucleic acid is typically operatively linked to a regulatory sequence effective to express the polypeptide encoded by the nucleic acid.

The polypeptide can be isolated from cell lysates, cell supernatants, and/or cell culture medium a variety of suitable techniques known in the art, including, e.g., various chromatography of cell lysates and/or cell supernatants. For example, the polypeptide can be isolated from cell lysates and/or cell culture medium by first concentrating the culture medium using centrifugal filters (Amicon), alternatively, by precipitating the polypeptides with ammonium sulfate or polyethylene glycol and then resuspending the polypeptides in PBS or other suitable buffers. The polypeptide can then be purified using either size-exclusion chromatography on Sephacryl S-400 column (Amersham Biosciences) as described in, e.g., Hjorth, R. and J. Moreno-Lopez, J. Virol. Methods 5:151-158 (1982), or another affinity chromatography, or by centrifugation through 20-60% sucrose gradients as described in, e.g., Konish et al., Virology 188:714-720 (1992). Fractions containing the desired polypeptides can be identified by ELISA or SDS-PAGE followed by protein silver stain and immunoblotting. The desired fractions are pooled and further concentrated. Sucrose in gradient centrifugation fractions can be removed using PD-10 column (Amersham Biosciences) gel filtration. Additional purification techniques include those described in the Examples below and hydrophobic interaction chromatography (Diogo, M. M, et al., J. Gene Med. 3:577-584 (2001)), and any other suitable technique known in the art.

Any suitable purification technique that is known in the art can also be used. Polypeptide purification methods known in the art include those set forth in, e.g., Sandana (1997) BIOSEPARATION OF PROTEINS, Academic Press, Inc., Bollag et al. (1996) PROTEIN METHODS, 2^{nd} Edition Wiley-Liss, NY, Walker (1996) THE PROTEIN PROTOCOLS HANDBOOK Humana Press, NJ, Harris and Angal (1990) PROTEIN PURIFICATION APPLICATIONS: A PRACTICAL APPROACH IRL Press at Oxford, Oxford, England, Scopes (1993) PROTEIN PURIFICATION: PRINCIPLES AND PRACTICE 3^{rd} Edition Springer Verlag, NY, Janson and Ryden (1998) PROTEIN PURIFICATION: PRINCIPLES, HIGH RESOLUTION METHODS AND APPLICATIONS, Second Edition Wiley-VCH, NY; and Walker (1998) PROTEIN PROTOCOLS ON CD-ROM Humana Press, NJ. Cells suitable for polypeptide production are known in the art and are discussed elsewhere herein (e.g., Vero cells, 293 cells, BHK, CHO, and COS cells can be suitable). Cells can be lysed by any suitable technique including, e.g., sonication, microfluidization, physical shear, French press lysis, or detergent-based lysis.

In one aspect, the invention provides a method of purifying a polypeptide of the invention (e.g., a recombinant gp120 polypeptide variant), which comprises transforming a suitable host cell with a nucleic acid of the invention (e.g., a recombinant nucleic acid that encodes a recombinant polypeptide comprising the polypeptide sequence of SEQ ID NO:1) in the host cell (e.g., a CHO cell or 293 cell), lysing the cell by a suitable lysis technique (e.g., sonication, detergent lysis, or other appropriate technique), and subjecting the lysate to affinity purification with a chromatography column comprising a resin that includes at least one novel antibody of the invention (usually a monoclonal antibody of the invention) or antigen-binding fragment thereof, such that the lysate is enriched for the desired polypeptide (e.g., a polypeptide comprising the polypeptide sequence of SEQ ID NO:1).

In another aspect, the invention provides a method for purifying such target polypeptides, which method differs from the above-described method in that a nucleic acid comprising a nucleotide sequence encoding a fusion protein that comprises a polypeptide of the invention (e.g., SEQ ID NO:1) and a suitable tag (e.g., an e-epitope/his tag), and purifying the polypeptide by immunoaffinity, lentil-lectin affinity column chromatography, immobilized metal affinity chromatography (IMAC), or metal-chelating affinity chromatography (MCAC) enrichment techniques. Additional purification methods are disclosed elsewhere herein.

In another aspect, the invention provides a method of producing a polypeptide of the invention, which method comprises introducing into a population of cells a recombinant expression vector comprising a nucleic acid of the invention, culturing the cells in a culture medium under appropriately sufficient conditions for expression of the nucleic acid from the vector and production of the polypeptide encoded by the nucleic acid, and isolating the polypeptide from the cells, culture medium, or both. The cells chosen are based on the desired processing of the polypeptide and based on the appropriate vector (e.g., E. coli cells are preferred for bacterial plasmids, whereas 293 cells are preferred for mammalian shuttle plasmids and/or adenoviruses, particularly E1-deficient adenoviruses).

In yet another aspect, the invention includes a method of producing a polypeptide, the method comprising: (a) introducing into a population of cells a recombinant expression vector comprising at least one nucleic acid of the invention the encodes a polypeptide of the invention; (b) administering the expression vector into a mammal; and (c) isolating the polypeptide from the mammal or from a byproduct of the mammal.

A polypeptide of the invention can also be produced by culturing a cell or population of cells of the invention (which, e.g., have been transformed with a nucleic acid of the invention that encodes such polypeptide) under conditions sufficient for expression of the polypeptide and recovering the polypeptide expressed in or by the cell using standard techniques known in the art.

In addition to recombinant production, the polypeptides of the invention may be produced by direct peptide synthesis using solid-phase techniques (see, e.g., Stewart et al. (1969) SOLID-PHASE PEPTIDE SYNTHESIS, W.H. Freeman Co, San Francisco and Merrifield J. (1963) J. Am. Chem. Soc. 85:2149-2154). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. For example, subsequences may be chemically synthesized separately and combined using chemical methods to produce a polypeptide of the invention or fragments thereof. Alternatively, synthesized polypeptides may be ordered from any number of companies that specialize in production of polypeptides. Most commonly, polypeptides of the invention are produced by expressing coding nucleic acids and recovering polypeptides, e.g., as described above.

The invention includes a method of producing a polypeptide of the invention comprising introducing a nucleic acid of the invention, a vector of the invention, or a combination thereof, into an animal, such as a mammal (including, e.g., rat, nonhuman primate, bat, marmoset, pig, or chicken), such that a polypeptide of the invention is expressed in the animal, and the polypeptide is isolated from the animal or from a byproduct of the animal. Isolation of the polypeptide from the animal or animal byproduct can be by any suitable technique, depending on the animal and desired recovery strategy. For example, the polypeptide can be recovered from sera of mice, monkeys, or pigs expressing the polypeptide of the invention. Transgenic animals (including the aforementioned mammals) comprising at least one nucleic acid of the invention are provided by the invention. The transgenic animal can have the nucleic acid integrated into its host genome (e.g., by an AAV vector, lentiviral vector, biolistic techniques performed with integration-promoting sequences, etc.) or can have the nucleic acid in maintained epichromosomally (e.g., in a non-integrating plasmid vector or by insertion in a non-integrating viral vector). Epichromosomal vectors can be engineered for more transient gene expression than integrating vectors. RNA-based vectors offer particular advantages in this respect.

Also provided is method of producing an isolated polypeptide of the invention which comprises introducing a nucleic acid encoding said polypeptide into a population of cells in a medium, which cells are permissive for expression of the nucleic acid, maintaining the cells under conditions in which the nucleic acid is expressed, and thereafter isolating the polypeptide from the medium.

Some secreted recombinant gp120 polypeptide variants of the invention are secreted more efficiently than a WT HIV-1 gp120 polypeptide. Analysis of polypeptide or protein secretion can be performed by any suitable technique. For example, secretion levels can be determined by comparing the results of a Western blots/immunoblots performed with the supernatant of cells transfected with polynucleotides encoding such polypeptides and similar supernatants obtained from cells transfected with polynucleotides expressing a corresponding WT gp120 polypeptide, where both such recombinant and WT polypeptides are expressed from a substantially identical expression cassette (e.g., an expression cassette comprising or consisting essentially of an identical promoter, enhancer, and polyA region sequences), such as the pMAmp vector described herein. Measuring the expression level of a recombinant gp120 polypeptide variant of the invention or a recombinant WT gp120 polypeptide can be carried out by any suitable technique as discussed in detail above in the section entitled “Vectors, Vector Components, and Expression Systems.”

Antibodies

The present invention also provides novel or recombinant antibodies that are useful in a number of respects, including, e.g., diagnostic, therapeutic, or prophylactic uses. For example, in one aspect, the invention provides at least one antibody induced in response to the administration or expression of at least one polypeptide of the invention (e.g., at least one polypeptide comprising a sequence having at least 85, 86, 87, 88, 90, 91, 92, 93, 94, 95, 96, 97, 98, or 99% sequence identity to a polypeptide sequence selected from the group consisting of SEQ ID NOS:1-21 and 56-63).

In another aspect, the invention provides a population of such antibodies, expressed by antibody-producing cells (e.g., human B cells) in response to the administration and/or expression of at least one such polypeptide of the invention in an area where such polypeptide can induce such an immune response from such antibody-producing cells.

In another aspect, the invention provides at least one monoclonal antibody that binds to a polypeptide of the invention. Such a monoclonal antibody(ies) typically is produced by a hybridoma that is generated by the fusion of an antibody-producing cell exposed to a polypeptide of the invention by administration or expression near the antibody-producing cell.

In another aspect, the invention provides an isolated antibody (or population of antibodies) or antisera that specifically binds a polypeptide of the invention, such as a polypeptide comprising a polypeptide sequence selected from the group consisting of SEQ ID NOS:1-21 and 56-63. The antibody may be a monoclonal antibody. Also provided is an antibody or antiserum produced by administering at least one polypeptide of the invention to a mammal. An immortalized cell line that produces any antibody of the invention is also contemplated, as is an immortalized cell line comprising at least one polypeptide of the invention.

In another aspect of the invention, a polypeptide of the invention (or antigenic or immunogenic fragment thereof) is used to produce one or more antibodies which have, e.g., diagnostic, therapeutic, or prophylactic uses, e.g., related to the activity, distribution, and expression of polypeptides and fragments thereof. Antibodies can be induced following expression of the polypeptide encoded by a nucleic acid of the invention.

Antibodies to polypeptides of the invention may be generated by methods well known in the art. Such antibodies may include, but are not limited to, polyclonal, monoclonal, chimeric, humanized, single chain, Fab fragments and fragments produced by a Fab expression library. Antibodies, e.g., those that block receptor binding, may be employed for therapeutic and/or prophylactic use.

Polypeptides for antibody induction do not require biological activity; however, the polypeptides or peptides should be antigenic. Peptides used to induce specific antibodies may have an amino acid sequence consisting of at least about 10 amino acids, 15 or 20 amino acids, or 25 or 30 amino acids. Short stretches of a polypeptide may be fused with another protein, such as keyhole limpet hemocyanin, and antibody produced against the chimeric molecule.

Antibodies of the invention can be characterized by the ability to detectably bind to an HIV virus, such as an HIV-1 virus or an HIV gp120 polypeptide, such as an HIV-1 gp120 polypeptide. Such antibodies may be capable of binding 1, 2, 3, 4, 5, 6, 7, 8, 9, or 10 or more HIV viruses (e.g., HIV-1) of the same and/or different subtypes. Some such antibodies may be able to facilitate an immune response against an HIV virus in a subject to whom an effective amount of such antibodies are administered.

Also provided is a hybridoma that expresses an antibody that binds to at least one HIV-1 virus or HIV-1 gp120 envelope polypeptide and a method of producing such a hybridoma. The method of producing such a hybridoma includes the steps of exposing an antibody-producing cell (e.g., a spleen B cell in a mammalian host or mammalian host tissue) to a polypeptide of the invention for a suitable period of time, fusing the antibody-expressing B cell to a myeloma cell (usually a selectable “tumor partner” myeloma cell), using standard hybridoma generation techniques (e.g., PEG-induced fusion—see, e.g., METHODS IN ENZYMOLOGY: IMMUNOCHEMICAL TECHNIQUES, PART I: HYBRIDOMA TECHNOLOGY AND MONOCLONAL ANTIBODIES, Langone et al. (Eds.), Academic Press (1997) and HYBRIDOMA TECHNOLOGY IN THE BIOSCIENCES AND MEDICINE, Springer, Plenum Pub. Corp. (1985) for discussion and other techniques). Included are hybridomas that express monoclonal antibodies that bind at least one HIV-1 virus or HIV-1 gp120 envelope polypeptide with high optical density values (as measured, e.g., in an ELISA) and/or with efficient production.

Also included are methods of producing such antibodies. For example, such antibodies can be produced, e.g., by administering an effective amount (e.g., an antigenic or immunogenic amount) of at least one polypeptide of the invention or an antigenic or immunogenic fragment thereof, or an effective amount of a vector or nucleic acid encoding such at least one such polypeptide, or composition comprising an effective amount of such at least one polypeptide or nucleic acid or polynucleotide encoding said at least polypeptide, to a suitable animal host or host cell. The host cell is cultured or the animal host is maintained under conditions permissive for formation of antibody-antigen complexes. Subsequently produced antibodies are recovered from the cell culture, the animal, or a byproduct of the animal (e.g., sera from a mammal). The production of antibodies can be carried out with either at least one polypeptide of the invention, or a peptide or polypeptide fragment thereof comprising at least 10, 15, 20, 30, 50, 75, or 100 amino acids in length. Alternatively, a nucleic acid or vector can be inserted into appropriate cells, which are cultured for a sufficient time and under periods suitable for transgene expression, such that a nucleic acid sequence of the invention is expressed therein resulting in the production of antibodies that bind to the recombinant antigen encoded by the nucleic acid sequence. Antibodies thereby obtained can have diagnostic and/or prophylactic uses. Such antibodies, and compositions and pharmaceutical compositions comprising such antibodies (by use of the principles described above with respect to other compositions and pharmaceutically acceptable compositions), are features of the invention.

Additional methods of producing polyclonal and monoclonal antibodies are known to those of skill in the art. See, e.g., Current Protocols in Immunology, John Colligan et al., eds., Vols. I-IV (John Wiley & Sons, Inc., NY, 1991 and 2001 Supplement), and Harlow and Lane (1989) ANTIBODIES: A LABORATORY MANUAL, Cold Spring Harbor Press, NY, Stites et al. (eds.) BASIC AND CLINICAL IMMUNOLOGY (4^{th} ed.) Lange Medical Publications, Los Altos, Calif., and references cited therein, Goding (1986) MONOCLONAL ANTIBODIES: PRINCIPLES AND PRACTICE (2d ed.) Academic Press, New York, N.Y., and Kohler and Milstein (1975) Nature 256:495-497. Other suitable techniques for antibody preparation include selection of libraries of recombinant antibodies in phage or similar vectors. See Huse et al. (1989) Science 246:1275-1281; Ward et al. (1989) Nature 341:544-546. Specific monoclonal and polyclonal antibodies and antisera will usually bind with a K_{D} of at least about 0.1 μM, 0.01 μM, or 0.001 μM.

Detailed methods for preparation of chimeric (humanized) antibodies can be found in, e.g., U.S. Pat. No. 5,482,856. Additional details on humanization and other antibody production and engineering techniques can be found in Borrebaeck (ed.) (1995) Antibody Engineering, 2^{nd} Edition Freeman and Company, NY (Borrebaeck); McCafferty et al. (1996) Antibody Engineering, A Practical Approach IRL at Oxford Press, Oxford, England (McCafferty), and Paul (1995) Antibody Eng'g Protocols Humana Press, Towata, N.J. (Paul).

Humanized antibodies are especially desirable in applications where the antibodies are used as therapeutics and/or prophylactics in vivo in mammals (e.g., such as humans) and ex vivo in cells or tissues that are delivered to or transplanted into mammals (humans). Human antibodies consist of characteristically human immunoglobulin sequences. The human antibodies of this invention can be produced in using a wide variety of methods (see, e.g., Larrick et al., U.S. Pat. No. 5,001,065, and Borrebaeck McCafferty and Paul, supra, for a review). In one embodiment, the human antibodies of the present invention are produced initially in trioma cells. Genes encoding the antibodies are then cloned and expressed in other cells, such as nonhuman mammalian cells. The general approach for producing human antibodies by trioma technology is described by Ostberg et al. (1983), Hybridoma 2:361-367, U.S. Pat. Nos. 4,634,664 and 4,634,666. The antibody-producing cell lines obtained by this method are called triomas because they are descended from three cells—two human and one mouse. Triomas have been found to produce antibody more stably than ordinary hybridomas made from human cells.

Additional useful techniques for preparing antibodies are described in, e.g., Gavilodono et al., Biotechniques 29(1):128-32, 134-6, and 138 (passim) (2000), Nelson et al., Mol. Pathol. 53(3):111-7 (2000), Laurino et al., Ann. Clin. Lab. Sci. 29(3):158-66 (1999), Rapley, Mol. Biotechnol. 3(2):139-54 (1995), Zaccolo et al., Int. J. Clin. Lab. Res. 23(4):192-8 (1993), Morrison, Annu. Rev. Immunol. 10:239-65 (1992), “Antibodies, Annigene, and Molecular Mimiery,” Meth. Enzymol. 178 (John J. Langone, Ed. 1989), Moore, Clin. Chem. 35(9):1849-53 (1989), Rosalki et al., Clin. Chim. Acta 183(1):45-58 (1989), and Tami et al., Am. J. Hosp. Pharm. 43(11):2816-25 (1986), as well as U.S. Pat. Nos. 4,022,878, 4,350,683, and 4,022,878. A technique for producing antibodies with remarkably high binding affinities is provided in Border et al., Proc. Natl. Acad. Sci. USA 97(20):10701-05 (2000).

The invention also provides an antibody fusion protein, wherein an antibody of the invention is expressed as a fusion protein with an anti-tumor cytokine (e.g., TNF-αc) and/or a pro-coagulant factor.

In general, the polypeptides of the invention provide structural features that can be recognized, e.g., in immunological assays. The production of antisera comprising at least one antibody (for at least one antigen) that binds or specifically binds a polypeptide of the invention, and the polypeptides that are bound by such antisera, are features of the invention. Binding agents, including the novel antibodies described herein, may bind a polypeptide of the invention with an affinity of about 1×10² M⁻¹ to about 1×10¹² M⁻¹ (i.e., about 10⁻²-10⁻¹² M) or greater, including a range of from about 10⁴ M⁻¹ to 10¹¹ M⁻¹, about 10⁶ M⁻¹ to 10¹⁰

M⁻¹, about 10⁵ M⁻¹ to about 10¹¹ M⁻¹, about 10⁷ M⁻¹ to 10⁹ M⁻¹, about 10⁸ M⁻¹ to 10¹⁰ M⁻¹, or about 10⁸ M⁻¹ to 10⁹ M⁻¹. Conventional hybridoma technology can be used to produce antibodies having affinities of up to about 10⁹ M⁻¹. However, other technologies, including phage display and transgenic mice, can be used to achieve higher affinities (e.g., up to at least about 10¹² M⁻¹). In many aspects of the invention a higher binding affinity is advantageous. However, in other aspects, discussed elsewhere herein, lower affinities are preferred. As noted previously, the binding affinity of a ligand and a receptor (e.g., between an antibody and antigen) can be measured by standard techniques known to those of skill in the art.

In order to produce antiserum or antisera for use in an immunoassay, at least one antigenic or immunogenic polypeptide (or antigenic or immunogenic polypeptide-encoding polynucleotide) of the invention is produced and purified as described herein. For example, a polypeptide of the invention may be produced in a mammalian cell line. Alternatively, a rabbit or an inbred strain of mice can immunized with the antigenic or immunogenic polypeptide(s) in combination with a standard adjuvant, such as Freund's adjuvant or alum, and a standard mouse immunization protocol (see Harlow and Lane, supra, for a standard description of antibody generation, immunoassay formats and conditions that can be used to determine specific immunoreactivity). Alternatively, at least one polypeptide derived from at least one polypeptide sequence disclosed herein or expressed from at least one polynucleotide sequence disclosed herein can be conjugated to a carrier protein and used as an immunogen for the production of antiserum. Polyclonal antisera typically are collected and titered against the antigenic or immunogenic polypeptide in an immunoassay, for example, a solid phase immunoassay with one or more of the antigenic or immunogenic proteins immobilized on a solid support. In the above-described methods where novel antibodies and antisera are provided, antisera resulting from the administration of the polypeptide (or polynucleotide and/or vector) with a titer of about 10⁶ or more typically are selected, pooled and subtracted with the control co-stimulatory polypeptides to produce subtracted pooled titered polyclonal antisera.

Some antisera raised or induced by an immunizing antigen are not totally specific for their inducing antigen, but bind related (cross-reacting) antigens, either because the cross-reacting antigens share epitopes, or the epitopes are sufficiently similar in shape or structure to bind the same antibody. David Male, IMMUNOLOGY: AN ILLUSTRATED OUTLINE (Gower Medical Publishing, London & NY, 1986).

Some antibodies of the invention can cross-react with one or more HIV-1 viruses or pseudoviruses, with one or more WT HIV-1 gp120 envelope proteins, and/or with one or more antigenic or immunogenic polypeptide sequences of the invention (e.g., a polypeptide comprising a polypeptide sequence having at least 90, 91, 92, 93, 94, 95, 96, 97, 98, 99, or 100% sequence identity to a polypeptide sequence selected from the group consisting of SEQ ID NOS:1-21 and 56-63). Cross-reactivity of a population of antibodies and/or a particular antibody can be determined using standard techniques, such as competitive binding immunoassays and/or parallel binding assays, and standard calculations for determining the percent cross-reactivity. Usually, where the percent cross-reactivity is at least 5-10× as high for the test polypeptides, the test polypeptides are said to specifically bind the pooled subtracted antisera or antibody. That polypeptides, nucleic acids, recombinant cells, and vectors of the invention are able to induce the production of a population of antibodies that cross-react (i.e., bind both) with multiple HIV viruses of the same or different subtypes or combination thereof is an important feature of the invention. Another significant feature attendant the polypeptides, nucleic acids, vectors, and cells of the invention is an ability to induce a cross-reactive T cell-mediated immune response, such as a T cell proliferative immune response against multiple HIV viruses of the same or different subtypes or combination thereof.

In yet another aspect, the invention provides anti-idiotype antibodies related to antibodies produced in response to an antigenic or immunogenic polypeptide of the invention. An anti-idiotype antibody will usually bear the internal image of the Ab₁ epitope-recognition site (i.e., the image of the antigen-binding site of an antibody raised against, e.g., an immunogenic polypeptide of the invention) and, as such, can often mimic the immunological properties of the portion of the antigen comprising the recognized epitope(s). Techniques for the production of anti-idiotype antibodies are known. Briefly, the invention provides a method of producing such an antibody comprising providing an Ab₁ antibody, as described above (e.g., a murine hybridoma cell monoclonal antibody to a polypeptide comprising or consisting essentially of SEQ ID NO:1), introducing such an antibody to a tissue system or host comprising antibody-producing cells, wherein the Ab₁ antibody is foreign (e.g., to a human tissue, goat, or other mammal) to produce the anti-idiotype antibody. Alternatively, hybridomas that produce such antibodies can be generated by exposure of a suitable type of hybridoma to the antibody. Such antibodies can be subject to modification or fragmentation as described above with respect to other antibodies of the invention.

In a further aspect, the invention provides an anti-anti-idiotype antibody and a method for producing the same. Anti-anti-idiotype antibodies can be produced by exposing an anti-idiotype antibody of the invention to a foreign host or host tissue comprising antibody-producing cells, and isolating resulting antibodies, or through the use of hybridomas generated from such cells (to produce monoclonal anti-anti-idiotype antibodies). Anti-anti-idiotype antibodies comprise a portion that resembles the epitope recognition sequence of an Ab₁ antibody and can be used in a manner similar to such antibodies of the invention.

Such anti-idiotype and anti-anti-idiotype antibodies of the invention are useful inasmuch as human antibodies to mouse or other non-human mammal Ab₁ antibodies do not induce production of human anti-mouse antibodies during therapeutic administration.

Compositions

The invention further provides novel and useful compositions comprising at least one polypeptide, nucleic acid, vector, virus, pseudovirus, VLP, cell, and/or antibody of the invention, or any combination thereof. Such a composition can comprise a carrier, excipient, or diluent. Such a composition can comprise any suitable amount of any suitable number of polypeptides, nucleic acids, vectors, viruses, pseudoviruses, VLPs, cells, and/or antibodies of the invention. Also provided are pharmaceutical compositions comprising at least one polypeptide, nucleic acid, vector, virus, pseudovirus, VLP, cell, or antibody of the invention, or any combination thereof, and a pharmaceutically acceptable carrier, excipient, or diluent.

For example, in one embodiment, the invention provides composition comprising an excipient or carrier and at least one polypeptide of the invention (e.g., 1, 2, 3, 4, 5, 6, 7, 8, 9, 10 more polypeptides), wherein the at least one polypeptide is present in the composition in an amount effective to induce in a subject to whom the composition has been administered an antibody and/or T cell immune response against at least one HIV-1 virus of the same or different subtypes. The composition may induce s in the subject a neutralizing antibody response against at least one HIV-1 virus of the same or of different subtype (or any combination of subtypes thereof). Corresponding pharmaceutical compositions comprising a pharmaceutically acceptable excipient or carrier and at least one such polypeptide are also provided.

In another aspect, the invention provides compositions (including pharmaceutical compositions) that comprise an excipient or carrier (or pharmaceutically acceptable excipient or carrier) and at least one nucleic acid of the invention (e.g., 1, 2, 3, 4, 5, 6, 7, 8, 9, 10 or more nucleic acids), wherein the nucleic acid is present in the composition in an amount effective to induce in a subject to whom the composition has been administered an antibody and/or T cell immune response against at least one HIV-1 virus of the same or different subtypes. The composition may induce s in the subject a neutralizing antibody response against at least one HIV-1 virus of the same or of different subtype (or any combination of subtypes thereof). Corresponding pharmaceutical compositions comprising a pharmaceutically acceptable excipient or carrier and at least one such nucleic acid are also provided.

A pharmaceutical composition of the invention may comprise a pharmaceutically acceptable excipient or carrier and an antigenic or immunogenic amount of at least one polypeptide, nucleic acid, vector, virus, or VLP of the invention (or a combination thereof) effective to induce a immune response to at least one HIV-1 virus in a subject to whom the pharmaceutical composition is administered. The immune response may comprise a neutralizing antibody and/or T cell response against one, two, three, or more HIV-1 viruses of the same or different subtypes (or any combination of subtypes thereof).

The composition (or pharmaceutical composition) can be any non-toxic composition that does not interfere with the antigenicity or immunogenicity of the at least one polypeptide, nucleic acid, vector, virus, pseudovirus, VLP, or cell of the invention included therein. The composition can comprise one or more excipients or carriers, and the pharmaceutical composition comprises one or more pharmaceutically acceptable carriers. A wide variety of acceptable carriers, diluents, and excipients are known in the art and can be included in the compositions and pharmaceutical compositions of the invention. For example, a variety of aqueous carriers can be used, e.g., buffered saline, such as PBS, and the like are advantageous in injectable formulations of the polypeptide, nucleic acid, vector, virus, pseudovirus, VLP, and/or cell of the invention. Such solutions are preferably sterile and generally free of undesirable matter. Compositions may be sterilized by conventional, well-known sterilization techniques. Compositions of the invention may comprise pharmaceutically acceptable auxiliary substances, as required, to approximate physiological conditions. Such substances include, e.g., pH adjusting agents, buffering agents, and toxicity adjusting agents, including, for example, sodium acetate, sodium chloride, potassium chloride, calcium chloride, sodium lactate, and the like. Any suitable carrier can be used in the administration of a polypeptide, nucleic acid, vector, virus, VLP, and/or cell of the invention. Numerous carriers for administration of therapeutic proteins are known in the art.

Compositions of the invention, including pharmaceutical compositions, can also include diluents, fillers, salts, buffers, detergents (e.g., a nonionic detergent, such as Tween-80), stabilizers (e.g., sugars or protein-free amino acids), preservants, tissue fixatives, solubilizers, and/or other materials suitable for inclusion in a pharmaceutically composition. Examples of suitable components of the pharmaceutical composition are described in, e.g., Berge et al., J. Pharm. Sci. 66(1):1-19 (1977), Wang and Hanson, J. Parenteral. Sci. Tech. 42:S4-S6 (1988), U.S. Pat. Nos. 6,165,779 and 6,225,289, and elsewhere herein. Pharmaceutical compositions also can include preservatives, antioxidants, and/or other additives known to those of skill in the art. Examples of suitable pharmaceutically acceptable carriers for use in the pharmaceutical compositions are described in, e.g., Urquhart et al., Lancet 16:367 (1980), Lieberman et al., PHARMACEUTICAL DOSAGE FORMS —DISPERSE SYSTEMS (2^{nd} ed., Vol. 3, 1998), Ansel et al., PHARMACEUTICAL DOSAGE FORMS & DRUG DELIVERY SYSTEMS (7^{th} ed. 2000), Martindale, THE EXTRA PHARMACOPEIA (31^{st} edition), Remington's PHARMACEUTICAL SCIENCES (16^{th}-20^{th} editions), THE PHARMACOLOGICAL BASIS OF THERAPEUTICS, Goodman and Gilman, Eds. (9^{th} ed.-1996), WILSON AND GISVOLDS TEXTBOOK OF ORGANIC MEDICINAL AND PHARMACEUTICAL CHEMISTRY, Delgado and Remers, Eds. (10^{th} ed.-1998), and U.S. Pat. Nos. 5,708,025 and 5,994,106. Principles of formulating pharmaceutically acceptable compositions are described in, e.g., Platt, Clin. Lab Med. 7:289-99 (1987), Aulton, PHARMACEUTICS: THE SCIENCE OF DOSAGE FORM DESIGN, Churchill Livingstone (New York) (1988), EXTEMPORANEOUS ORAL LIQUID DOSAGE PREPARATIONS, CSHP (1998), and “Drug Dosage,” J. Kans. Med. Soc. 70(1):30-32 (1969). Additional pharmaceutically acceptable carriers particularly suitable for administration of vectors are described in, e.g., Int'l Patent Appn Publ. No. WO 98/32859.

The composition or pharmaceutical composition of the invention can comprise or be in the form of a liposome. Suitable lipids for liposomal formulation include, without limitation, monoglycerides, diglycerides, sulfatides, lysolecithin, phospholipids, saponin, bile acids, and the like. Preparation of such liposomal formulations is described in, e.g., U.S. Pat. Nos. 4,837,028 and 4,737,323.

The form of the compositions or pharmaceutical composition can be dictated, at least in part, by the route of administration of the polypeptide, nucleic acid, vector, virus, pseudovirus, VLP, or cell of interest. Because numerous routes of administration are possible, the form of the pharmaceutical composition and its components can vary. For example, in transmucosal or transdermal administration, penetrants appropriate to the barrier to be permeated can be included in the composition. Such penetrants are generally known in the art, and include, for example, for transmucosal administration, detergents, bile salts, and fusidic acid derivatives. In contrast, in transmucosal administration can be facilitated through the use of nasal sprays or suppositories.

A common administration form for compositions of the invention, including pharmaceutical compositions, is by injection. Injectable pharmaceutically acceptable compositions typically comprise one or more suitable liquid carriers such as water, petroleum, physiological saline, bacteriostatic water, Cremophor ELTM (BASF, Parsippany, N.J.), PBS, or oils. Liquid pharmaceutical compositions can further include physiological saline solution, dextrose (or other saccharide solution), polyols, or glycols, such as ethylene glycol, propylene glycol, PEG, coating agents which promote proper fluidity, such as lecithin, isotonic agents, such as mannitol or sorbitol, organic esters such as ethyoleate, and absorption-delaying agents, such as aluminum monostearate and gelatins. The injectable composition can be in the form of a pyrogen-free, stable, aqueous solution. An injectable aqueous solution may comprise an isotonic vehicle such as sodium chloride, Ringer's injection solution, dextrose, lactated Ringer's injection solution, or an equivalent delivery vehicle (e.g., sodium chloride/dextrose injection solution). Formulations suitable for injection by intraarticular, intravenous, intramuscular, intradermal, subdermal, intraperitoneal, and subcutaneous routes, include aqueous and non-aqueous, isotonic sterile injection solutions, which can include antioxidants, buffers, bacteriostats, and solutes that render the formulation isotonic with the blood of the intended recipient (e.g., PBS and/or saline solutions, such as 0.1 M NaCl), and aqueous and non-aqueous sterile suspensions that can include suspending agents, solubilizers, thickening agents, stabilizers, and preservatives.

The administration of a polypeptide, nucleic acid, vector, virus, pseudovirus, VLP or cell of the invention (or a composition of the invention comprising any such component) can be facilitated by a delivery device formed of any suitable material. Examples of suitable matrix materials for producing non-biodegradable administration devices include hydroxapatite, bioglass, aluminates, or other ceramics. In some applications, a sequestering agent, such as carboxymethylcellulose (CMC), methylcellulose, or hydroxypropylmethylcellulose (HPMC), can be used to bind the polypeptide, nucleic acid, vector, virus, VLP or cell to the device for localized delivery.

A nucleic acid or vector of the invention can be formulated with one or more poloxamers, polyoxyethylene/polyoxypropylene block copolymers, or other surfactants or soap-like lipophilic substances for delivery of the nucleic acid or vector to a population of cells or tissue or skin of a subject. See e.g., U.S. Pat. Nos. 6,149,922, 6,086,899, and 5,990,241.

Nucleic acids and vectors of the invention can be associated with one or more transfection-enhancing agents. In some embodiments, a nucleic acid and/or nucleic acid vector of the invention typically is associated with one or more stability-promoting salts, carriers (e.g., PEG), and/or formulations that aid in transfection (e.g., sodium phosphate salts, dextran carriers, iron oxide carriers, or biolistic delivery (“gene gun”) carriers, such as gold bead or powder carriers). See, e.g., U.S. Pat. No. 4,945,050. Additional transfection-enhancing agents include viral particles to which the nucleic acid or nucleic acid vector can be conjugated, a calcium phosphate precipitating agent, a protease, a lipase, a bipuvicaine solution, a saponin, a lipid (e.g., a charged lipid), a liposome (e.g., a cationic liposome), a transfection facilitating peptide or protein-complex (e.g., a poly(ethylenimine), polylysine, or viral protein-nucleic acid complex), a virosome, or a modified cell or cell-like structure (e.g., a fusion cell).

Nucleic acids and vectors of the invention can also be delivered by in vivo or ex vivo electroporation methods, including, e.g., those described in U.S. Pat. Nos. 6,110,161 and 6,261,281 and Widera et al., J. of Immunol. 164:4635-4640 (2000).

Transdermal administration of at least one recombinant polypeptide, nucleic acid, vector, virus, pseudovirus, VLP and/or cell of the invention can be facilitated by a transdermal patch comprising such component in any suitable composition in any suitable form. Such transdermal patch devices are provided by the invention. For example, at least one polypeptide, nucleic acid, vector, virus, pseudovirus, VLP, and/or cell can be contained in a liquid reservoir in a drug reservoir patch device, or, alternatively, the polypeptide, nucleic acid, vector, virus, pseudovirus, VLP, and/or cell can be dispersed throughout a material suitable for incorporation in a simple monolithic transdermal patch device. Typically, the patch comprises an immunogenic or antigenic amount of the polypeptide, nucleic acid, vector, virus, pseudovirus, VLP, and/or cell—such as an amount effective to induce an immune response in a subject contacted with the patch. Examples of such patch devices are known in the art. The patch device can be either a passive device or a device capable of iontophoretic delivery of at least one polypeptide, nucleic acid, vector, virus, pseudovirus, VLP, and/or cell to the skin or tissue of the subject. Methods of promoting immunity to at least one HIV-1 virus in a subject comprise administering such a transdermal patch to the skin of the subject for a period of time and under conditions sufficient to promote immunity to at least one HIV-1 virus.

The composition, particularly the pharmaceutical composition, may comprise an amount of at least one polypeptide, nucleic acid, vector, virus, pseudovirus, VLP, and/or cell in an amount or dose effective to induce in a subject to whom it is (they are) administered an immune response against HIV. Preferably, the induced response is one that inhibits or protects against HIV-1 infection in the subject following administration of the composition. The composition can comprise any suitable dose or amount of the at least one polypeptide, nucleic acid, vector, virus, pseudovirus, VLP, and/or cell (i.e., component) sufficient to induce the desired immune response. Proper dosage can be determined by any suitable technique and considerations for determining the proper are known in the art. In a simple dosage testing regimen, low doses of the composition are administered to a test subject or system (e.g., an animal model, cell-free system, or whole-cell assay system). Dosage is commonly determined by the efficacy of the particular component to be administered, the condition of the subject, the body weight of the subject, and/or target area of the subject to be treated. The size of the dose is also determined by the existence, nature, and extent of any adverse side-effects that accompany the administration of any such particular component in a particular subject. Principles related to dosage of therapeutic and prophylactic agents are provided in, e.g., Platt, Clin. Lab Med. 7:289-99 (1987), “Drug Dosage,” J. Kans. Med. Soc. 70(1):30-32 (1969), and other references described herein (e.g., Remington's, supra).

By way of example, an effective amount of a polypeptide of the invention for an initial dosage may be about 100 μg-10 mg, 100-600 μg, usually about 300-500 μg (e.g., about 400 or 500 μg), which dosage can be by any suitable protocol, e.g., such as administered at about 0, 2, 4, and 6 weeks, e.g., through electroporation or a subcutaneous, i.m., or i.p. injection. Such a composition may further comprise an adjuvant. A polypeptide of the invention is typically administered as a soluble polypeptide (such as a fusion protein comprising a polypeptide of the invention covalently linked to an Ig molecule)).

An effective amount of an antibody of the invention may be about 500 mg for an initial dose to a human. Such antibody dose can be formulated in PBS and/or in an adjuvant such as Freund's incomplete adjuvant or alum. Normally, such a dose will be followed by subsequent administrations of smaller doses (e.g., about 100-400 mg) about ever 2-3 days or week for a period of months. In some situations, a period of higher initial doses over several (e.g., 5) consecutive days can be used (e.g., 5 consecutive daily doses of about 400-450 mg antibody). Additionally or alternatively, about 300-500 mg can be administered every 4-6 weeks thereafter the initial dosage of antibody.

By way of example, a composition comprising an effective amount of a nucleic acid of the invention typically comprises from about 0.1 μg to about 50 mg of at least one nucleic acid of the invention, including about 0.5 μg to about 45 mg, about 1 μg to about 30 mg, about 1 μg to about 25 mg, about 1 μg to about 20 mg, about 1 μg to about 15 mg, about 1 μg to about 10 mg, about 500 μg to about 10 mg, about 500 μg to about 5 mg, about 1 mg to about 10 mg, about 1 mg to about 5 mg, about 2 mg to about 5 mg, about 1 μg to about 2 mg, including about 1 μg to about 1 mg, about 1 μg to about 500 μg, 1 μg to about 100 μg, 1 μg to about 50 μg, and 1 μg to about 10 μg of the nucleic acid. For delivery of a vector comprising a nucleic acid of the invention, the same amount(s) can be administered. The composition administered to a subject may comprise about 1, 2, 5, or 10 mg of a nucleic acid or vector of the invention. A mixture of two or more nucleic acids of the invention (or mixture of two or more vectors, each encoding a nucleic acid of the invention) can be administered in such amount(s). The volume of carrier or diluent in which such nucleic acid is administered depends upon the amount of nucleic acid to be administered. For example, 2 mg nucleic acid is typically administered in a 1-ml volume of carrier or diluent. The amount of nucleic acid in the composition depends on the host to which the nucleic acid composition is to be administered, the characteristics of the nucleic acid (e.g., gene expression level as determined by the encoded peptide, codon optimization, and/or promoter profile), and the form of administration. For example, biolistic or “gene gun” delivery methods of as little as about 1 μg of nucleic acid dispersed in or on suitable particles is effective for inducing an immune response even in large mammals such as humans. In some instances, biolistic delivery of at least about 5 μg, 10 μg, or more of the nucleic acid may be desirable. Biolistic delivery of nucleic acids is discussed further elsewhere herein.

For injection of a nucleic acid composition, a larger dose of nucleic acid typically will be desirable. In general, an injectable nucleic acid composition comprises at least about 1 μg, 5 μg, 25 μg, 30 μg, 50 μg, 75 μg, 80 μg, 100 μg, 150 μg, 500 μg, 1 mg, 2 mg, 5 mg, 10 mg, 25 mg, or 30 mg nucleic acid. In some instances, the injectable nucleic acid composition comprises about 0.25-5 mg nucleic acid, typically in a volume of diluent, carrier, or excipient of about 0.5-1 ml. An injectable nucleic acid solution can comprise about 0.5 mg, 1 mg, 1.5 mg, or 2 mg nucleic acid in a volume of about 0.25 ml, 0.5 ml, 0.75 ml, or 1 ml. In one format, 2 mg nucleic acid is administered in a 1 ml volume of carrier, diluent, or excipient (e.g., PBS or saline). However, in some instances, lower injectable doses (e.g., less than about 5, 4, 3, 2, or 1 μg) of the nucleic acid of the invention are about equally or more effective in producing an antibody response than the above-described higher doses. Following administration of one or more nucleic acids of the invention, one or more polypeptides of the invention may optionally be administered (e.g., as a protein boost) in a dose(s) ranging from about 0.01 mg to about 10 mg, including 0.1 mg to 5 mg, or 0.5 mg to 1 mg protein. Each polypeptide is typically delivered in a composition comprising PBS and, if desired, an adjuvant, such as Alum. Such composition optionally has a pH of 7.4. DNA and protein immunizations are typically administered independently and at 4-week intervals.

Effective doses of a nucleic acid vector of the invention (e.g., a pMAmp monocistronic or bicistronic vector) normally range from about 1-15 mg (including, e.g., 1, 2, 5, 8, or 10 mg). Nucleic acid vectors are usually delivered in a concentration of about 2, 5, or 10 mg/ml. As an example, one such method comprises administering to a subject a first dose of 10 mg DNA vector comprising 5 mg of a nucleotide sequence encoding the polypeptide of SEQ ID NO:1. The first dose is optionally followed by a second dose of the DNA vector administered to the subject about 4 weeks after the first dose, followed by one or more protein boosts, each of which is administered to the subject approximately 4 weeks after the previous nucleic acid or protein administration. A protein boost may comprise the protein encoded by the nucleic acid (e.g., homologous protein boost) or another protein of the invention (e.g., heterologous protein boost) in an amount of about 400 or 500 μg protein in a 0.5-2 ml solution. Two rounds of DNA-DNA-protein immunizations at 4-week intervals may be administered. Polypeptides encoded by nucleic acids typically (although not necessarily) also include a functional signal peptide sequence (e.g., SEQ ID NO:52 or 55) as described above. A nucleic acid vector of the invention (e.g., pMAmp vector) is typically formulated in sterile PBS at pH 7.4. A polypeptide of the invention is typically formulated in sterile PBS, optionally with alum or another adjuvant, and optionally at pH 7.4.

Also included is a composition comprising a first nucleic acid encoding an antigenic or immunogenic polypeptide of the invention (e.g., a polypeptide comprising a polypeptide sequence having at least 90% identity to one or more of the sequences set forth in SEQ ID NOS:1-21 and 56-63) and a second nucleic acid encoding a second antigenic or immunogenic polypeptide of the invention (e.g., a polypeptide comprising a polypeptide sequence having at least 90% identity to one or more of the sequences set forth in SEQ ID NOS:1-21 and 56-63), wherein the first nucleic acid and second nucleic acid encode polypeptides comprising different amino acid sequences and each polypeptide independently induces an immune response against one or more HIV-1 viruses. The invention also includes a composition comprising a pool or library of such nucleic acids.

Also provided is a viral vector composition, which comprises a carrier or excipient and a viral vector of the invention. Pharmaceutical compositions comprising a pharmaceutically acceptable carrier or excipient and a viral vector are also provided. The amount or dosage of viral vector particles or viral vector particle-encoding nucleic acid depends on: (1) the type of viral vector particle with respect to origin of vector, including, but not limited to, e.g., whether the vector is an alphaviral vector, Semliki-Forest viral vector, adenoviral vector, adeno-associated (AAV) viral vector, flaviviral vector, papillomaviral vector, and/or herpes simplex viral (HSV) vector, (2) whether the vector is a transgene expressing or recombinant peptide displaying vector, (3) the host, and (4) other considerations discussed above. Generally, with respect to gene transfer vectors, the pharmaceutically acceptable composition comprises at least about 1×10² viral vector particles in a volume of about 1 ml (e.g., at least about 1×10² to 1×10⁸ particles in about 1 ml). Higher dosages also can be suitable (e.g., at least about 1×10⁶, about 1×10⁸, about 1×10⁹, about 1×10¹⁰ particles/ml).

Nucleic acid compositions of the invention comprise can comprise one or more additional nucleic acids encoding non-gp120 proteins. For example, a nucleic acid can be co-administered with a second immunostimulatory nucleotide sequence or a second cytokine- or adjuvant-encoding nucleotide sequence, such as a nucleotide sequence encoding an IFN-gamma, interleukin, GM-CSF, or human B7-1 or B7-2 protein.

The invention further provides a composition comprising a carrier or excipient (e.g., pharmaceutically acceptable carrier or excipient) and at least one VLP formed from at least one polypeptide of the invention. Such composition may comprise an amount of VLP effective to induce in a subject to whom it is administered an immune response against at least one HIV-1 virus or that inhibits or reduces HIV-1 infection. Dosage considerations for VLPs are similar to those described above with respect to viral vector particles and other compositions of the invention. Pharmaceutical compositions comprising a pharmaceutically acceptable carrier or excipient and at least one VLP are also provided.

The invention also provides a composition comprising an aggregate of two or more polypeptides of the invention. Moreover, the invention provides a composition comprising a population of one or more multimeric polypeptides of the invention.

Vaccines

The present invention provides vaccines that may have the ability to induce both specific antibodies and/or T cells against at least one HIV-1. In one aspect, the invention provides vaccines that comprise at least polypeptide, nucleic acid, vector, virus, VLP or cell of the invention. Some such vaccines may be useful in prophylactic methods for inhibiting, reducing, or protecting against HIV-1 infection in a subject not yet exposed to such HIV-1 virus. Some such vaccines may be useful in therapeutic methods for inhibiting or reducing HIV-1 infection in a subject already exposed to or infected with such HIV-1 virus. Various vaccine formats are contemplated. Exemplary vaccines comprise: (1) at least one polypeptide of the invention (e.g., SEQ ID NOS: 1-21, 56-63, 107-110 and 131-134); (2) at least one nucleic acid of the invention (e.g., SEQ ID NOS:23-50 and 64-79); (3) at least one vector comprising at least one nucleic acid of the invention (e.g., SEQ ID NOS:23-50 and 64-79); (4) at least one virus comprising at least one nucleic acid and/or at least one polypeptide of the invention; (5) at least one VLP comprising at least one polypeptide of the invention; and (6) at least one cell comprising at least one nucleic acid or polypeptide of the invention. Such vaccines may optionally include an adjuvant. Such vaccines may be formulated as compositions that further include an excipient or carrier or as pharmaceutical compositions that further include a pharmaceutically acceptable excipient or carrier.

Dosage and Administration: In one embodiment, a single dose of a DNA sequence of the invention (such as that set forth SEQ ID NO:23) (i.e., DNA vaccine) is typically about 10 mg; a single dose of a protein of the invention (such as that set forth in SEQ ID NO:1) (protein vaccine) is typically about 500 μg. Each dose may be administered to a subject by injection. The immunization schedule may comprise two or more rounds each of DNA-DNA-Protein immunizations. Alternatively, an effective amount of the protein is administered 2-6 times, at intervals to be determined, and no DNA is administered. One of skill will understand that other immunization protocols and formats can be utilized. A gp120 variant of the invention may be administered to a subject as a DNA vector encoding a gp120 polypeptide variant (e.g., administered by injection) followed by a second administration of a DNA vector encoding a gp120 polypeptide variant (e.g., administered by injection) after an appropriate interval of time. If desired, the second DNA vector administration may be followed (after an appropriate interval of time) by the administration of a gp120 polypeptide variant (e.g., administered by injection). See, e.g., discussion of exemplary DNA/DNA/protein immunization schedules above. Alternatively, a gp120 variant may be administered only as a gp120 polypeptide variant—without administration of the corresponding DNA vector. Multiple administrations of the protein or DNA vector may be given at appropriate intervals.

A bicistronic DNA vector encoding both a gp120 polypeptide variant of the invention and an immunomodulatory molecule (e.g., a co-stimulatory molecule, such as B7-1, or a cytokine, such as GM-CSF) can also be utilized. Such a bicistronic vector is administered first at a 10-mg total DNA dose. Alternatively, the gp120 variant and the immunomodulatory molecule can be administered via two separate DNA vectors; in this case, each vector is administered in a 5-mg dose. After a desired period of time following the first DNA immunization, a second identical DNA immunization is given using the bicistronic vector. If desired, after a desired period of time, the second DNA immunization is followed by administration of 500 μg of a gp120 protein variant (e.g., the recombinant polypeptide variant shown in SEQ ID NO:1). This round of immunization is optionally followed by one or more additional rounds of DNA/DNA/protein boost immunizations. Immunizations are typically performed by injection.

Formulation: The DNA vaccine (e.g., DNA expression vector encoding a gp120 protein variant) is formulated in sterile, phosphate-buffered saline at a pH 7.4. The protein vaccine (e.g., gp120 protein variant) is formulated in a suitable amount of an adjuvant (e.g., Alum) sufficient to augment an immune response (e.g., antibody titer) induced by administration of the DNA and/or protein vaccine alone.

In an exemplary embodiment, the DNA vector comprises the gp120 variant-encoding nucleotide sequence shown in SEQ ID NO:37, and the gp120 polypeptide variant comprises the sequence shown in SEQ ID NO:1. A subject is immunized by administering the DNA variant sequence once via injection or other suitable delivery method (e.g., electroporation, gene gun, impressing through the skin, lipofection). After a desired period of time, the subject is immunized again via injection with the same DNA sequence in the same amount. After a desired period of time, the gp120 protein variant is administered to the subject via injection. A second round of identical DNA-DNA-protein immunizations can be administered to the subject if desired. The doses of nucleic acid and protein for each immunization can be the same as those discussed above or can be varied. In such administration format, the vaccine induces high titers of anti-HIV-1 antibodies in rabbits, including antibodies that cross-react with two or more HIV-1 viruses or pseudoviruses. The protein boost augments the HIV-specific immune response(s) induced by the DNA vaccine alone.

Among other uses, the vaccine may have the ability to prevent or inhibit HIV-1 infection in subjects not previously infected with an HIV-1 virus. In subjects exposed to an HIV-1 virus, the vaccine may have the ability to reduce the initial dose of virus transferred, and thus to prolong the median time to development of an HIV-1 related disease or AIDS. The above vaccine approaches may overcome limitations of current approaches for preventing or inhibiting HIV-1 infection or inhibiting or preventing HIV-1 related disease (including) AIDS or the progression of such disease.

Kits

The present invention also provides kits including one or more of the polypeptides, nucleic acids, vectors, viruses, VLPs, pseudoviruses, cells, vaccines, and/or compositions of the invention. Kits of the invention optionally comprise: (1) at least one polypeptide, nucleic acid, vector, virus, pseudovirus, VLP, cell, vaccine, or composition of the invention, optionally mixed with one or more adjuvants; (2) instructions for practicing any method described herein, including a therapeutic or prophylactic method, instructions for using any component identified in (1); (3) a container for holding said at least one such component or composition, and/or (4) packaging materials. One or more of the polypeptides, nucleic acids, vectors, viruses, pseudoviruses, VLPs, cells, vaccines, and/or compositions of the invention can be packaged in packs, dispenser devices, and kits for administration to a subject, such as a mammal. The polypeptides, nucleic acids, vectors, viruses, pseudoviruses, VLPs, cells, and/or vaccines can be formulated with an excipient or carrier (including, e.g., a pharmaceutically acceptable excipient or carrier), thereby forming a composition (including, e.g., pharmaceutical composition). Packs or dispenser devices that comprise one or more unit dosage forms are provided. Typically, instructions for administration of the compounds are provided with the packaging, along with a suitable indication on the label that the compound is suitable for treatment of an indicated condition. For example, the label may state that the active compound within the packaging is believed useful in prophylactic methods for inhibiting or protecting against HIV-1 infection and/or therapeutic methods for inhibiting or reducing HIV-1 infection as discussed elsewhere herein.

### EXAMPLES

The following examples further illustrate the invention, but should not be construed as limiting its scope in any way.

### Example 1

### A. Cloning of Parental HIV-1 gp120 Envelope Gene Sequences for In vitro Recombination

The parental gene sequences selected for in vitro DNA recombination should introduce adequate diversity, be similar enough to ensure sufficiently extensive recombination, and represent the various functionalities of the gene family. We focused on different HIV-1 subtype B primary-isolate strains that were completely sequenced and readily available. These strains were chosen based on their relatedness, viral phenotype, and co-receptor usages. These ten strains include both syncytium-inducing (SI) and non-syncytium-inducing (NSI) phenotypes and different co-receptor usages.

TABLE 5
Subtype B HIV-1 Strains used for in vitro DNA recombination

| | GenBank Accession | | Co-receptor |
|---|---|---|---|
| Viral Strain | Number | Phenotype* | Usage |
| JRCSF | AY426125 | NSI | R5 |
| 89.6 | U39362 | SI | R5X4 |
| 92HT593 (“593”) | AY669721 | SI | R5X4 |
| 92HT594 (“594”) | U08445 | SI | R5X4 |
| 92HT596 (“596”) | U08446 | SI | R5X4 |
| 92HT599 (“599”) | U08447 | SI | X4 |
| 92US657 (“657”) | U04908 | NSI | R5 |
| 92US712 (“712”) | AY669725 | NSI | R5 |
| 92US727 (“727”) | U79720 | NSI | R5 |
| 93US073 (“073”) | AY669727 | NSI | R5 |

*NSI: non-syncytium inducing; SI: syncytium inducing
R5 refers to the chemokine receptor CCR5; X4 refers to the chemokine receptor CXCR4; R5X4 refers to dual chemokine receptors CCR5 and CXCR4.

The envelope gp 160 gene for each of these HIV-1 subtype B primary-isolated strains can be synthesized by using published nucleotide sequence provided in GenBank (see, e.g., Table 5) using standard nucleotide synthesis procedures or cloned from genomic DNA isolated from the corresponding HIV-1 strain-infected human PBMCs using standard polymerase chain reaction (PCR) procedures.

Each HIV-1 gp160 protein comprises a gp120 envelope protein and a gp41 envelope protein. Based on the obtained envelope gp160-encoding nucleotide sequences, nucleic acids encoding the gp120 full-length Env polypeptide and gp120 core Env polypeptide of each wild-type HIV-1 strain were generated by PCR and cloned into a pMAmp vector. A wild-type full-length gp120 polypeptide comprises the complete gp120 polypeptide sequence of a wild-type HIV-1 envelope protein. For example, the wild-type full-length gp120 polypeptide sequence of HIV-1 gp120-HXB2 is shown in FIGS. 10A-10F (SEQ ID NO:54). Full-length WT gp120 polypeptide-encoding constructs were utilized as parental sequences for in vitro DNA recombination. The amino acid sequences of the full-length parental gp120 polypeptides shown in Table 5 are provided in SEQ ID NOS:80-89.

The corresponding “core” form (gp120 core) of each wild-type full-length gp120 polypeptide was designed based on the deletion construct model used for the crystallization studies by Kwong et al., Nature 393:648-659 (1998) and Wyatt et al., Nature 393:705-711 (1998) and prepared using a PCR-base approach. To generate a gp120 core polypeptide, portions of the C1 and C5 regions were partially deleted. In addition, the V1/V2 and V3 loop residues were replaced with Gly-Ala-Gly tripeptides; the conserved bases of the V1/V2 and V3 loops formed by the disulfide bond remain intact. Once the gp120 core polypeptide constructs were designed, nucleic acids encoding these WT gp120 core polypeptide constructs were designed and constructed using a PCR-based approach as discussed above. The amino acid sequences of the core forms of the parental gp120 polypeptides shown in Table 5 (except for 92US727) are set forth in SEQ ID NOS:90-98.

The uppermost sequence of the alignment shown in FIGS. 10A-10F is the polypeptide sequence of an exemplary parental full-length wild-type gp120 polypeptide, designated HXB2 gp120 (SEQ ID NO:54). The corresponding core form of this full-length gp120 sequence comprises the following elements from N terminus to C terminus: 1) a first amino acid segment of the parental HIV-1 gp120 HXB2 envelope protein, which corresponds to amino acids 83-127; 2) a first GAG tripeptide, the N terminal of which is covalently linked to C terminal of the first amino acid segment; 3) a second amino acid segment of the parental HIV-1 gp120 HXB2 envelope protein, which corresponds to amino acids 195-297, wherein the N terminal of the second segment is covalently linked to the C terminal of the first GAG tripeptide; 4) a second GAG tripeptide, wherein the N terminal of the second GAG tripeptide is covalently linked to the C terminal of the second amino acid segment; and 5) a third amino acid segment of the parental HIV-1 gp120 HXB2 envelope protein, which corresponds to amino acids 330-492, wherein the N terminal of the third amino acid segment is covalently linked to the C terminal of the second GAG tripeptide. Residues 83-127 of gp120-HXB2 represents part of the C1 domain. Residues 83-127 of gp120-HXB2 represents part of the C1 domain, residues 195-297 of gp120-HXB2 represent the C2 domain, and residues 330-492 of gp120-HXB2 represent the C3, V4, C4, and V5, domains and part of C5 domain.

### B. pMAmp Expression Vector Used for Protein Expression

The 10 parental nucleic acids encoding gp120 full-length Env polypeptides and 9 parental nucleic acids encoding gp120 core Env polypeptides were cloned into the mammalian expression vector termed “pMAmp” shown in FIG. 1. This vector can be used for expression in mammalian cells of any nucleic acid of interest, including any nucleic acid encoding an gp120 full-length polypeptide variant or gp120 core polypeptide variant of the invention. This vector is based on pcDNA3 (Invitrogen) and is suitable for use in cell culture and DNA-based immunization in animals. A nucleic acid encoding an HIV-1 gp120 full-length or gp120 core polypeptide of interest is cloned between the Agel and NgoMIV restriction sites using standard techniques known in the art. If desired, a nucleic acid sequence of interest can be codon-optimized based on codons chosen from highly expressed human genes (Haas, J., et al., Curr. Biol. 6:315-24 (1996)) prior to being cloned into the vector.

Each vector was named according to the gp120-encoding nucleotide sequence cloned into the vector. For example, vectors comprising a WT parental full-length gp120-encoding nucleic acid from various HIV-1 strains were designated as: pMAmp-gp120-30 JRCSF; pMAmp-gp120-93US073; pMAmp-gp120-92HT593; pMAmp-gp120-92HT594; pMAmp-gp120-92HT596; pMAmp-gp120-92HT599; pMAmp-gp120-92US657; pMAmp-gp120-92US712; pMAmp-gp120-92US727; and pMAmp-gp120-89.6.

### C. DNA Preparation, Sequencing and Sequence Analysis

Plasmid DNA was purified using Qiaprep Spin Miniprep Kits (Qiagen) and quantitated with DU640 Spectrophotometer (Beckman). DNA sequencing was performed using ABI BigDye 3.1 chemistry on an ABI 3700 DNA analyzer. Synthetic genes are verified by DNA sequencing using multiple primers to produce overlapping sequence reads. The raw sequence data are quality controlled and assembled into contigs using appropriate software. The number of times that a given nucleotide is read in the sequence is included in the quality control process.

Sequence alignment and similarity for both nucleotide and amino acid sequences were analyzed in Vector NTI 9.0 (Invitrogen). Phylogenetic and molecular evolutionary analyses were conducted using MEGA version 3.0. See Kumar, S. et al, Brief Bioinform 5, 150-163 (2004). To avoid any artificial homology, all gp120 protein sequences used, subtype B parents and the recombined variants, were mature peptide sequences without leader peptide, histidine-tag and linker sequences. Sequences were aligned in Vector NTI 9.0 (Invitrogen) and the alignment file exported in MSF format. Each file was unlocked in BioEdit version 7.0.5 (freeware by Tom Hall, Ibis Therapeutics), saved in FASTA format, opened and converted into MEGA format for construction of phylogenetic trees using the neighbor-joining method and tested by bootstrap analysis using 1,000 replications. Kumar, S. et al, Brief Bioinform. 5, 150-163 (2004).

### D. Numbering of Amino Acid Residues in HIV Proteins

To identify the position or location of an amino acid residue in an HIV protein, including, e.g., in a wild-type protein or polypeptide of the invention (such as, for example, in an gp120 polypeptide variant or gp120 core polypeptide variant), the guidelines outlined by Korber et al., “Numbering Positions in HIV Relative to HXB2,” supra (see the Los Alamos HIV Sequence Database at hiv.lanl.gov/content/hiv-db/num-hxb2/numbering.html) can be used. These guidelines can also be used to identify the position or location of a nucleic acid residue in an HIV nucleic acid sequence, including, e.g., in an HIV genome sequence, wild-type HIV sequence, or in any nucleic acid of the invention, such as in nucleic acid encoding a gp120 full-length or core polypeptide variant.

Applying these guidelines, amino acid residues of wild-type gp120 polypeptides and gp120 polypeptide variants of the invention are numbered sequentially by reference to the amino acid residues of the HXB2 gp120 envelope protein (SEQ ID NO:54), which is also referred to herein as “gp120-HXB2”, as shown in FIGS. 10A-10F. In FIGS. 10A-10F, the sequence of gp120-HXB2 (SEQ ID NO:54) is numbered, beginning with the number 1 for the first amino acid residue of the sequence. FIGS. 10A-10F also shows representative polypeptides of the invention aligned with the gp120-HXB2 protein sequence. Numbering of each amino acid residue of a particular polypeptide is readily determined by reference to the amino acid position of the corresponding gp120-HXB2 amino acid residue using the numbering convention adopted by Los Alamos HIV Sequence Database. Korber et al., supra.

For example, the polypeptide sequence of the gp120 polypeptide variant ST-008 (SEQ ID NO:1) begins with the three amino residues GAA, which are aligned with and correspond to amino acid residues at position numbers 29-31 of the gp120-HXB2 protein sequence. By reference, amino acid residues GAA of ST-008 are designated with numbers 29-31, respectively.

A polypeptide sequence of interest may have a deletion of one or more amino acid residues relative to the gp120-HXB2 reference polypeptide sequence (SEQ ID NO:54). Suppose, e.g., a region of a polypeptide sequence of interest is aligned with the gp120-HXB2 sequence over gp120-HXB2 amino acid residue positions 30-40, but the sequence of interest has a two-amino acid residue deletion at positions 8-9 relative to the reference polypeptide sequence gp120-HXB2. To indicate this explicitly, such region of the polypeptide sequence of interest would be designated with the annotation 30-40(del 8-9). A polypeptide sequence of interest may have an insertion of one or more amino acid residues relative to the gp120-HXB2 reference polypeptide sequence. For example, ST-008 (SEQ ID NO:1) comprises a segment of 3 amino acids (GGD), which is positioned between amino acids at positions 138 (N) and 139 (N) of gp120-HXB2. This GGD segment does not exist in HXB2, as is indicated by the 3 dashes included in the gp120-HXB2 sequence shown in FIG. 10A. This GGD segment in the ST-008 (SEQ ID NO:1) is referred to as corresponding to gp120-HXB2 amino acid residue positions 139a-139c, with the residues of the GGD segment numbered as 139a, 139b, and 139c, respectively. These residues insertions may be designated by the notation 139a=G, 139b=G, and 139c=D.

The numbering of amino acid residues of WT gp120 core polypeptides and gp120 core polypeptides of the invention (e.g., L7-068, L7-084, L7-098, L7-028, L7-043, L7-010, and L7-105) can be determined similarly by alignment with and/or reference to the corresponding amino acid residue positions of gp120-HXB2, as shown in FIGS. 10A-10F.

### Example 2

### A. Generating Recombinant Libraries Using Multigene In Vitro Homologous DNA Recombination

Libraries of recombinant nucleic acid sequences encoding chimeric gp120 variants were generated by multigene in vitro recombination using the 10 purified wild-type HIV-1 recombinant gp120-encoding nucleic acids as parental nucleotide sequences. Recombination was carried out as essentially described in Stemmer, Proc. Natl. Acad. Sci. USA 91:10747-10751 (1994). The cloned env genes to be recombined are amplified by PCR and randomly fragmented to 50 and 200 base pairs. Various dilutions of the fragments are submitted to a PCR-like thermal cycling reaction (Id.; Minshull, J. et al., Methods 32:416-427 (2004)) to allow the chimeric recombined genes to reassemble by successive elongation reactions with Taq or equivalent DNA polymerases. Once gel analysis confirms an accumulation of products in the range of the expected size, the recombined genes are recovered by PCR using two primers that are positioned inside the original PCR primers used to prepare the fragments. The PCR products of this reaction are then digested with AgeI and NgoMIV, gel purified, and ligated at different ratios with the pMAmp vector digested with the same pair of enzymes. The ligation mixtures were used to transform Escherichia coli XL10-Gold® ultracompetent cells (Stratagene).

Bacterial colonies were picked using robotics and plasmid DNA was isolated from each clone using standard high-throughput (HTP) screening methods. The individual plasmid DNAs were transiently transfected into CHO cells (as discussed in greater detail below) to allow for expression and secretion of the shuffled gp120 polypeptide variants. The supernatants were used for the source of gp120 polypeptide variants, which were detected by a variety of human neutralizing and non-neutralizing monoclonal antibodies as described in greater detail below.

Expressed gp120 full-length polypeptides and wild-type HIV-1 full-length gp120 polypeptides were then screened for their respective abilities to bind or react with known human mAbs to wild-type HIV-1 gp120, including mAbs that are known to broadly neutralize HIV-1 (e.g., b12, 2G12) and HIV-1 non-neutralizing mAbs (e.g., b3 and b6) using the dot-immunoblotting method described in Example 4 below. The antigenicity characteristics of representative variants identified in the dot blots were analyzed further using the immunoprecipitation and surface plasmon resonance methods described in Example 4 below.

Nucleic acid constructs encoding the gp120 core polypeptides were designed and made as discussed above. DNA shuffling was similarly conducted using the nine nucleic acids encoding the nine wild-type HIV-1 gp120 core polypeptides. Expressed shuffled (chimeric) gp120 core polypeptide variants and wild-type HIV-1 gp120 core polypeptides were then screened for their respective abilities to bind or react with human mAbs to wild-type HIV-1 gp120 (e.g., 2G12, b12, b3, and b6) using dot-immunoblotting methods described in Example 4 below. The antigenicity characteristics of representative variants identified in the dot blots were analyzed further using the immunoprecipitation and surface plasmon resonance methods described in Example 4.

Polypeptide variants of the invention were also screened for their ability to induce a neutralizing antibody response after immunization of rabbits with plasmid DNA as described in Example 6.

### B. Further Rounds of Recombination and Screening

The basic module of in vitro DNA recombination (e.g., DNA shuffling) and screening comprised: (i) creating recombinant libraries of shuffled nucleic acids; (ii) prescreening with various human monoclonal antibodies known to broadly neutralize HIV-1; (iii) immunizing rabbits using electroporation-assisted DNA-based immunization techniques followed by heterologous protein boosting; (iv) purifying IgG from the individual test bleeds; and (v) conducting neutralization assays on primary HIV-1 viral isolates. Details of these procedures are presented elsewhere herein. This module (i.e., steps i-v above) was used to identify further antigens having antigenic and immunogenic properties of interest, including an ability to bind neutralizing and non-neutralizing HIV-1 antibodies and/or to induce a neutralizing antibody response against at least one HIV-1 virus.

Genes encoding shuffled gp120 variants chosen from the first round of recombination/shuffling are used to create libraries of chimeric genes. Wild-type parental gp120 genes may optionally be included in additional rounds of recombination/shuffling. The resultant libraries are then prescreened in vitro by transiently transfecting the shuffled genes into CHO cells in culture, recovering the expressed and secreted gp120 proteins in the supernatant, applying aliquots to membranes and characterizing them with respect to their ability to bind various monoclonal antibodies that broadly neutralize HIV-1. Selected molecules are then used to immunize rabbits to identify additional shuffled gp120 variants that can induce neutralizing antibodies against HIV-1, including those antigenic variants have the ability to induce an immune response against more HIV-1 viruses of the same subtype and/or against HIV-1 viruses of a greater number of different subtypes.

### C. Use of Codon-Optimized Nucleotide Sequences

Human codon usage has been shown to be typically superior for the expression of the HIV-1 env gene in mammalian cell systems (Haas et al., Current Biol. 6:315-324 (1996)). Env gp120 polypeptide variants identified in the first round of recombination/shuffling by screening procedures as having an ability to react with known human mAb(s) against HIV-1 and/or induce a neutralizing antibody response against HIV-1 primary isolates can be synthesized using an optimized human codon set, as discussed in Example 7, to maximize protein expression for protein production. Such synthetic gp120-coding nucleotide sequences comprising codons optimized for expression in human or mammalian cells usually have a high GC-nucleotide residue content.

Codon-optimized shuffled gp120 and gp120 core variants having antigenic or immunogenic properties of interest (e.g., which were positive for b12 binding and negative for binding with b3 and/or b6, or that induced a neutralizing immune response against at least one HIV-1) were used as parents for a further round of recombination/shuffling. To achieve both robustness and high fidelity in the enzymatic DNA shuffling reactions, Platinum® Pfx DNA polymerase (Invitrogen) was used. Recombinant libraries were produced comprising clones expressing additional chimeric gp120 full-length polypeptide variants or further chimeric gp120 core polypeptide variants exhibiting increased (positive) binding to b12 and decreased (negative) binding to b6. Antigenic prescreening of these libraries showed a further improvement in the proportion of b12-positive over the previous rounds. Among the b12 positive clones, over 50% of showed no reactivity to the non-neutralizing b6 monoclonal antibody in both shuffled libraries.

The use of codon-optimized nucleic acids allowed the production of high levels of recombinant protein, improved the sensitivity of the antigenicity prescreening assay, and improved the expression and thus immunogenicity of the genes when injected into rabbits.

### D. Library Evaluation and Maintenance

Before performing high throughput pre-screening, each library was evaluated for its expression and antigenicity using forty clones that were randomly chosen from a pilot transformation of the library ligation. Plasmids were digested with AgeI and NgoMIV to estimate the proportion of clones with inserts. Simultaneously these plasmids were transfected into CHO cells and the resultant supernatants were analyzed for gp120 secretion by dot blot using mAbs to the His tag and to gp120 (b3, b6, and b12). Libraries were processed further only if at least 30 clones (≧75%) had the correct insertion and 20 or more clones (≧50%) produced secreted protein as indicated by anti-His mAbs.

Transformed libraries were plated onto Q Trays (Genetix) and grown overnight. Individual colonies were identified, picked and inoculated with the aid of Q-Bot (Genetix) into the first ten columns of 96-well plates containing 0.1 ml LB broth with carbenicillin (50 μg/ml). Columns 11 and 12 of 96-well plates were left empty for controls to be added in the subsequent steps. After overnight incubation at 37° C., cultures were transferred into 96-deep-well blocks containing 1.2 ml of LB with carbenicillin (50 μg/ml) using a Multimek 96/384-Channel Automated Pipettor (Beckman Coulter) for plasmid preparation. Glycerol stocks of the library transformants were prepared. The 96-well block cultures were grown for 20 h at 37° C., and plasmid DNA was purified using Qiaprep 96 Turbo Miniprep Kits (Qiagen). DNA concentrations were determined using 96-well UV plates (Costar) on Spectra Max 190 (Molecular Devices) and normalized to 100 ng/μl using Tecan Genesis RSP 100.

### Example 3

This example illustrates procedures for generating stable mammalian cell lines and producing recombinant proteins from such cell lines.

### A. Gene Synthesis

Genes encoding gp120 polypeptides to be used to generate stable cell lines were synthesized by assembly PCR (Stemmer, W. et al., Gene 164:49-53 (1995)) from large numbers of overlapping 40-mer oligodeoxyribonucleotides using codons chosen from highly expressed human genes (Haas, J. et al., Curr. Biol. 6:315-24 (1996)). Errors that occurred during DNA synthesis were corrected using the QuikChange® Site-Directed Mutagenesis Kit (Stratagene). The synthetic genes were cloned into the pMAmp vector as described above.

### B. Cell Culture, Media, and Stable Cell Lines Expressing gp120 Polypeptides

Chinese Hamster Ovary (CHO)-K1 cells (ATCC No. CRL-61) were maintained in a 1:1 mixture of Dulbecco's Modified Eagle Medium and Nutrient Mixture F-12 (D-MEM/F-12; Invitrogen) supplemented with 10% (v/v) fetal bovine serum, 100 units/ml of penicillin (Invitrogen), and 100 μg/ml of streptomycin (Invitrogen), and incubated at 37° C. in a humidified incubator containing 5% CO₂ unless otherwise specified.

CHO-K1 cell lines stably expressing gp120 protein sequences were generated as follows. A mixture of 0.5 μg of the expression construct and 0.5 μg pPUR (Clontech Laboratories) containing the puromycin resistance gene was co-transfected into a 24-well culture of CHO-K1 cells using LipofectAMINE 2000 (Invitrogen) according to the manufacturer's instructions. One day after transfection, the cells were diluted (based on a predetermined rate of resistant colony formation) into 96-well plates in the presence of 10 μg/ml puromycin (Sigma). After 8-10 days of growth and selection in the presence of puromycin, the serum-containing medium was removed and the cells were covered with serum-free Opti-MEM I medium without puromycin for 3 days. The supernatants were then harvested and tested for the presence of secreted gp120 proteins by an automated dot-blot protocol (described below) followed by detection with the anti-Env polyclonal serum. The cells showing the highest expression levels were expanded to 24-well culture plates and then to T-75 culture flasks under puromycin selection pressure. Expression levels of expanded cultures were evaluated using Western blot as described above. The cell lines consistently exhibiting the highest expression levels were frozen for later use as production cell lines.

### C. Production of gp120 Proteins in CHO Cells

Stable CHO cells were thawed and passaged, and the gp120 expression levels were confirmed by Western blot before expansion into T-175 culture flasks (Falcon) in serum-containing medium with 10 μg/ml puromycin. After reaching confluency, each T-175 flask was transferred to a T-1750 roller bottle (Corning) containing 300 ml of serum-containing medium with 10 μg/ml puromycin. Two days post-inoculation, the medium was removed and replaced with 300 ml of fresh serum-containing medium for two additional days. Cells were then washed and covered with 300 ml of serum-free Opti-MEM I. The entire volume of culture supernatant was harvested every 24 hours and replaced with an equivalent volume of fresh serum-free Opti-MEM I medium for up to 14 days. Daily harvests of the supernatant were centrifuged at 3,400 rpm for 20 minutes at room temperature, pooled, and stored at −80° C. until purification.

### D. Affinity Purification of gp120 Proteins

As discussed above, the gp120 proteins are expressed with an N-terminal hexahistidine tag. The N-terminal hexahistidine tag allows the gp120 proteins to be readily purified by one-step Metal-Chelating Affinity Chromatography with a 5 ml Hi-Trap Ni-columns (Amersham Biosciences, Piscataway, N.J. or GE Healthcare) running on a BioLogic LP Chromatography System (BioRad, Hercules, Calif.). The stored culture supernatant was thawed, filtered through a 0.22 μm membrane, and concentrated 20-fold using Centricon® PLUS-80 Centrifugal Filter Units with a PL-10 membrane (Millipore). The concentrate was then loaded onto a Hi-Trap column freshly charged with Ni⁺⁺, washed with 20 mM NaH₂PO₄ (pH 7.4) containing 500 mM NaCl, pre-eluted with the same buffer containing 0.025 M imidazole, and eluted with 0.25 M imadazole in the same buffer. The progress of the chromatographic run was monitored through UV recording of the column effluent. The peak of eluted protein was collected and concentrated to a final concentration of approximately 1 mg/ml. Gel electrophoresis was used to verify the purity of the preparation. A single relatively homogenous band representing at least 90% or 95% of the material on the gel is taken as an acceptable level of purity of proteins for use in the immunization experiments.

In the course of purifying different recombinant gp120 proteins, we observed that metal-chelating affinity chromatography occasionally does not produce protein of sufficient purity (>90%) for immunization. In those instances, batch absorption of the protein preparation on High Q anion exchange resin (Bio-Rad) was usually sufficient to remove most of the residual impurities from recombinant gp120 and gp120 core protein preparations. If impurities remained after this step, an additional step of absorption with hydroxyapatite CHT II resin (Bio-Rad) was optionally used to achieve the desired level of purity. Under both conditions, gp120 remained unbound. For the surface plasmon resonance analysis, all proteins were further purified using High Q anion exchange resin and hydroxyapatite CHT II resin to achieve a minimum of 95% purity. Other strategies to remove impurities from envelope protein reparations, such as those described in Srivastava et al., J. Virol. 77:11244-11259 (2003), can also be employed.

Alternatively, gp120 proteins can be purified using a different purification protocol using two runs of lentil-lectin affinity chromatography (LLAC) because some histidine-tagged variants may not bind well to the metal-chelating affinity matrix. It is possible, although unlikely, that the hexahistidine tag is inaccessible in some protein constructs, and this would prevent such constructs from binding to the metal-chelating affinity column. The LLACs method requires two passes of gp120-containing CHO culture supernatant over the lentil-lectin affinity column (LLAC).

As an illustration, CHO-K1 cells stably expressing gp120 JRCSF were grown in T-75 flasks. The culture supernatant was collected, concentrated and loaded onto a Lentil lectin Sepharose 4B (Sigma) column using a BioLogic LP system (BioRad). After washing with 1 M NaCl, gp120 was recovered by washing with a buffer containing 500 mM of alpha-methyl-mannoside and 500 mM of alpha-methylglucoside (LLAC-1). Fraction analysis of LLAC-1 suggested that elution of the column by buffer containing alpha-methyl-mannoside and alpha-methylglucoside leads to the recovery of about 30% of the gp120 JRCSF. The remainder of the gp120 JRCSF actually bound to the lectin column initially but came off during the washing step (data not shown).

To maximize yield, we pooled the fractions from the first wash and applied them a second time to the lectin columns (LLAC-2); after elution this resulted in another 16% recovery of gp120 JRCSF (data not shown). The combined recovery of gp120-JRCSF was around 45%. A comparison of purified gp120 JRCSF from metal-chelating affinity chromatography (MCAC) and LLAC was performed. For each gel, one μg of each purified gp120 sample was separated on 4-12% SDS-PAGE followed by Coomassie Blue staining. For all gel analyses, Invitrogen SeeBlue pre-stained protein standards were loaded as molecular weight markers and one μg of gp120 IIIB (Protein Sciences) was included as a control (data not shown). Comparison of purified gp120 JRCSF from MCAC and LLAC revealed a similar purity, which was sufficient for the rabbit immunization methods discussed elsewhere herein.

### E. Prevention of Proteolytic Degradation of gp120

1. Characterization of gp120 Degradation in CHO-K1 Cells.

Cell associated V3-loop cleavage activity has previously been identified in T-lymphoid cell lines, primary lymphocytes and macrophages (Gu et al., AIDS Res. Hum. Retroviruses 9:1007-1015 (1993)) and is thought to contribute to HIV-1 infectivity (Kido et al., Biomed. Biochim. Acta 50:781-789 (1991); Stephens et al., Nature 343:219 (1990)). V3-loop specific cleavage of gp120 between the arginine and alanine of the GPGRAF in the V3 loop was reported during the development of a gp120 production process in CHO cells (Stephens et al., Nature 343:219 (1990)). The cleavage was found to be insignificant during a scale-up of roller bottle production and even absent after a 10- or 20-fold concentration of supernatant (Rhodes et al., J. Gen. Virol. 75(Pt 1):207-213 (1994); Scandella et al., AIDS Res. Hum. Retroviruses 9:1233-1244 (1993).

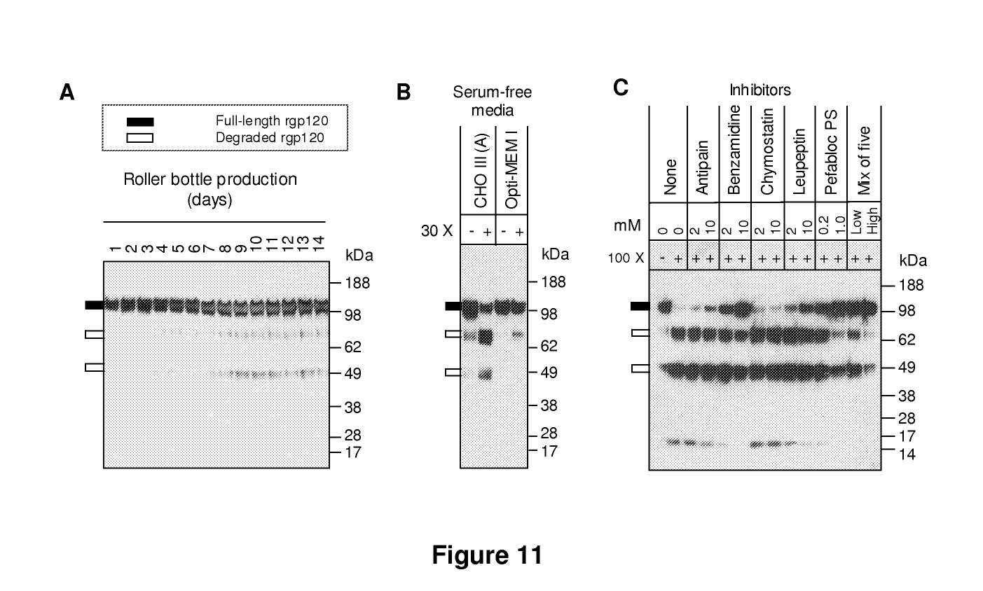

While developing a production process for multiple gp120 variants produced from stably transfected CHO-K1 lines, we occasionally observed some minor gp120 degradation using T-75 or T-175 flasks, characterized by two polypeptide fragments having sizes of 50 kDa and 70 kDa. However, when we scaled up to continuous culture using roller bottles, the degradation appeared more extensive and was found in most of the culture supernatants. To study the time course of the degradation activity, a stable CHO-K1 cell line expressing JRCSF gp120 was expanded to T-1750 roller bottles and protein production was carried out in serum-free CHO III (A) medium for 14 days. Equal amounts of the daily supernatant harvest were analyzed by Western blots to monitor gp120 expression (FIG. 11A). Throughout the 14 days of roller-bottle production, the gp120 level more than doubled from 7-8 mg/liter on day 1 to about 20 mg/liter on days 8-14 as estimated by Western blot analyses (FIG. 11A). The gp120 degradation activity was not observed on days 1 to 3, but became detectable on day 4 and reached plateau on day 9 (FIG. 11A). During the same period of time, the cell density increased significantly from a 100% confluent transparent single layer on day 1 to translucent multiple layers on day 14, in parallel with an increase in gp120 expression level and gp120 degradation activity.

The degradation became much more pronounced after the gp120 protein was purified by immobilized Ni-affinity chromatography. Gel analysis of the purified gp120 revealed that the degradation products were approximately 50 kDa and 70 kDa in size and were apparent only under reducing conditions (data not shown). The degraded gp120 had a molecular weight identical to intact gp120 under non-reducing conditions. Our results are similar to the observations reported by others (Clements, G. J., et al., AIDS Res. Hum. Retroviruses 7:3-16 (1991); Stephens, P. E., et al., Nature 343:219 (1990)), suggesting that a V3-specific proteolytic activity in CHO cell supernatant results in gp120 degradation. To confirm that the two degradation fragments co-purified from the Ni-affinity column were derived from N- and C-termini of gp120, respectively, partially degraded gp120 was analyzed for reactivity with an anti-His tag monoclonal antibody by Western blot analysis. While both degradation products reacted with the polyclonal anti-gp120 serum, only the 70 kDa polypeptide comprises the histidine tag and therefore is derived from N-terminus of gp120 (data not shown), matching our expectation of a V3-specific degradation of gp120.

2. Attempts to Separate Intact gp120 from Degraded gp120.

Linked by a disulfide bond, the unreduced degradation products are identical in molecular weight to intact gp120. As a result, the degradation products cannot be separated by gel-filtration chromatography except under reducing conditions (Scandella, C. J., et al., AIDS Res. Hum. Retroviruses 9:1233-44 (1993)). We tested whether the gp120 degradation products could be separated from the intact gp120 by using conventional anion-exchange chromatography and subsequent hydroxyapatite chromatography. Our results suggested that once V3-specific degradation occurred, it was not possible to separate the degraded gp120 from the intact gp120 using these chromatographic techniques.

3. Effects of Supernatant Concentration and Protease Inhibitors on gp120 Degradation.

Degradation of gp120 was relatively minor and sometime undetectable in culture supernatants (FIG. 11A). However, after purification, degradation of gp120 was more pronounced and purification appeared to induce complete degradation of intact gp120. This led us to hypothesize that the supernatant concentration step prior to purification might result in an increase in the local concentration of CHO-K1 protease components, which in turn might accelerate the degradation reaction. To test this hypothesis, aliquots of the day 14 supernatant that showed a minor but visible V3-loop degradation from the roller bottle production (FIG. 11A) were concentrated by 1-, 5-, 10-, 20-, 50-, and 100-fold. Two aliquots of 100 μl each of the concentrated samples were then prepared; one set was supplemented with a Sigma Protease Inhibitor Cocktail (Sigma), which includes five components, aprotinin, bestatin, leupeptin, E-64, and pepstatin A, at the recommended concentration, and the other set served as a control. The incubation was carried out for 10 hr at room temperature and then continued overnight at 4° C. At the end of incubation, all samples were brought back to the original supernatant volume using CHO III (A) serum-free medium and equal amounts corresponding to 10 μl of the starting supernatant volume were analyzed on Western blot. The results indicated that the degree of gp120 degradation is dependent on the supernatant concentration: the higher the concentration, the greater the extent of gp120 degradation (data not shown). After a 5- to 10-fold concentration, 50% or more of gp120 was degraded after incubation. A 100-fold concentration led to >95% degradation.

Adding the Sigma Protease Inhibitor Cocktail after concentration but prior to the incubation step partially blocked the proteolytic activity (data not shown), confirming the involvement of protease(s) in this process. The fact that the protection was not complete suggests either that a significant level of degradation occurred during the 30-minute concentration step before the addition of inhibitor cocktail, or that the inhibitor cocktail could not fully inhibit the proteolytic activity in the culture supernatant.

Two well-characterized V3-specific proteolytic activities identified in lymphatic cell lines belong to serine proteases (Kido, H., et al., J. Biol. Chem. 265:21979-85 (1990); Avril, L. E., et al., FEBS Lett. 317:167-72 (1993)). To determine if the CHO-cell associated gp120 V3 degradation activity also is caused by the same class of proteases, we chose a panel of five proteinase inhibitors (antipain, benzamidine, chymostatin, leupeptin, and Pefabloc PS; all from Sigma) based on their different specificities for serine proteases. We tested (1) each inhibitor individually at the two different concentrations and (2) one pool of all five inhibitors at one concentration and one pool of all five inhibitors at the second concentration. Selected concentrations were based principally on the study of Kido et al., J. Biol. Chem. 265:21979-85 (1990), which showed that antipain, leupeptin, and benzamidine at the higher concentrations we tested were able to inhibit at least 85% of the Arg degradation activity of tryptase TL₂ with a concentration equivalent to 20 mg/ml, and 62% of the Tyr degradation activity and 35% of the Arg degradation activity of chymostatin.

Each of the five protease inhibitors or a pool of them (at either the high or low concentration) was pre-incubated at room temperature for 1 hr with a 20 ml aliquot of the day 13 supernatant that showed a minor but visible V3-loop degradation from the roller bottle production (FIG. 11A). The mixtures were then concentrated 100-fold and incubated at room temperature for 16 hr. Controls lacking inhibitor, with and without a 100-fold concentration, were also incubated in parallel. At the end of the incubation period, all samples were brought back to the original supernatant volume using CHO III (A) serum-free medium. Equal amounts (corresponding to 10 μl of the start supernatant volume) were analyzed by Western blot. Concentrating the supernatant by 100-fold followed by room temperature incubation led to complete degradation of gp120 (FIG. 11C, compare lanes 1 & 2). Pre-incubation of supernatant with four inhibitors (antipain, benzamidine, leupeptin and Pefabloc PS) attenuated the gp120 degradation activity to different extents in a dose-dependent manner (FIG. 11C, compare lanes 3 & 4, 5 & 6, 9 & 10, and 11 & 12, respectively). Chymostatin showed no inhibitory effect (data no shown). Our results also indicated that a mixture of all five inhibitors at the higher concentrations tested completely prevented V3 degradation associated with 100-fold concentration of the supernatants (data not shown). From these tests of serine protease inhibitors, we concluded that the V3-degradation activity from CHO cell was not inhibited by chymostatin, a reversible inhibitor of chymotrypsin-like serine and some cysteine proteases, but rather by antipain, leupeptin, benzamidine and Pefabloc. While antipain and leupeptin are reversible inhibitors of cysteine and serine proteases, Pefabloc PS is an irreversible inhibitor of thrombin and other serine proteases, suggesting that the CHO cell V3-proteolytic activity may come from serine protease(s). The fact that benzamidine is a potent inhibitor of trypsin and trypsin-like enzymes further suggested that the V3-specific serine protease activity could be due to trypsin-like serine protease(s).

We concluded that the degradation of gp120 was localized to the V3 loop mainly based on molecular weight calculations of the degradation product and existing literature. Further substantiation came from a separate study in which we expressed both the JRCSF gp120 core molecule with deletions of V1, V2, and V3 and partial deletions in C1 and C5, which is homologous to what has been described by Kwong, P. D. et al., Nature 393:648-59 (1998), and a V3-containing JRCSF gp120 core, which is homologous to that described by Huang, C. C. et al., Science 310:1025-8 (2005). Only the V3-containing JRCSF gp120 core, but not gp120 core itself, showed a distinct and predictable degradation pattern. In addition, we successfully expressed over ten different HIV-1 subtype B gp120 core variants, none of which showed any sign of protein degradation throughout the expression, supernatant concentration, and purification process. All these observations further support the idea that our CHO-K1 protease activity targets the V3 variable loop of gp120.

4. Preventing Degradation of gp120.

Limited V3 degradation of gp120 has already occurred during roller-bottle production (FIG. 11A), suggesting that proteases accumulate during cell growth. Protease inhibitors are effective during supernatant processing. However, adding the inhibitors to cell culture is not a realistic choice, since the highest recommended working concentrations for mammalian cells are much less than optimal. Therefore, different strategies (such as medium optimization) must be investigated to minimized gp120 degradation at the production stage. Roller-bottle production of gp120 using stable CHO-K1 cell lines was performed for four days using two different serum-free media: CHO III (A) and Opti-MEM I. Aliquots from supernatants of both production runs were concentrated 30-fold and incubated at room temperature for 6 hr and than at 4° C. overnight. Aliquots of supernatants without concentration were incubated in parallel. At the end of incubation, all concentrated samples were brought back to the original supernatant volume using the serum-free medium corresponding to the production run, and equal amounts were analyzed by Western blot. The results showed that while use of CHO III (A) consistently resulted a detectable degradation of gp120, use of Opti-MEM prevented it without significantly affecting the expression level (FIG. 11B). After supernatant concentration, a lower level of gp120 degradation was observed in the run with Opti-MEM I than in the run with CHO III (A), suggesting that Opti-MEM I medium is less stimulatory for expression of the V3-specific protease(s) in CHO cells (FIG. 11B).

Having replaced CHO III (A) with Opti-MEM I, we were able to successfully scale up roller-bottle production of JRCSF gp120 to a total of 100 liters of supernatant without any detectable degradation of gp120. Based on the results of the inhibitor study (FIG. 11B), we established an efficient pre-incubation step using only a mixture of the two most potent inhibitors—benzamidine at 10 mM and Pefabloc PS at 1 mM—and purified >200 mg of protein. Use of optimized medium during production and use of potent protease inhibitors (e.g., a mixture of inhibitors) post-production can effectively prevent V3-specific degradation of gp120 by CHO cells. Use of a mixture of the two most potent proteinase inhibitors, benzamidine and Pefabloc PS as described above—e.g., 10 mM (2.2 g per liter) Benzamidine (Sigma) and 1 mM (0.24 g per liter) of Pefabloc (Sigma)—during gp120 protein production appears optimal. For example, by using optimized medium during production and the inhibitor mix post-production, we have been able to prevent V3-specific degradation for at least twenty HIV-1 subtype B gp120 variants expressed in CHO cells and to achieve milligram-level purification with a purity exceeding 90%. These procedures can be used to prevent V3-specific degradation of parental gp120 polypeptides and gp120 polypeptide variants of the invention that are expressed in CHO cells.

During production of these gp120 proteins, we also found that the degree of gp120 degradation seems to be intrinsic feature of different V3 sequences, with some gp120 sequences appearing to be more sensitive to degradation than others. This is expected, since it has been reported that the sequences surrounding these basic residues in the V3 loop, and possibly the conformation they form together with the GPxR sequence, can determine the sensitivity to cleavage (Schulz, T. F., et al., AIDS Res. Hum. Retroviruses 9:159-66 (1993)). The strain-to-strain difference in susceptibility of V3-specific degradation could also explain why only some reports in the literature mention proteolysis for gp120 molecules expressed in CHO cells (15, 22, 26). Accordingly, care needs to be taken in optimization of the scale-up method, day of harvest, production medium, protease inhibitor and supernatant processing method, to ensure an expression and purification of protein with an intact structure.

### Example 4

This example illustrates in vitro methods used to screen and characterize the antigenicity and antigenic phenotypes of parental gp120 proteins and gp120 protein variants in both full-length and core forms. Exemplary methods include dot-immunoblotting screening methods, immunoprecipitation assays, and surface plasmon resonance analyses—each of which utilizes human mAbs to assess the antibody binding characteristics of gp120 proteins.

### A. Human Monoclonal Antibodies

A variety of monoclonal antibodies (mAbs) can be used to characterize the mAb binding characteristics and reactivities of parental gp120 proteins and gp120 protein variants, including those shown in Table 6. Monoclonal antibody b12 recognizes the CD4 binding site (“CD4BS”) on env (Saphire et al., Science 293:1155-1159 (2001); Roben et al., J. Virol. 68:4821-4828 (1994); Burton et al., Science 266:1024-1027 (1994); Zhou et al., Nature 445 (7129):732-737 (2007)). Monoclonal antibody 2G12 reacts with carbohydrate moieties on gp120 (Trkola et al., J. Virol. 70:1100-1108 (1996); Scanlan et al., J. Virol. 76:7306-7321 (2002)). Both b12 and 2G12 neutralize HIV-1 relatively broadly. Two other CD4BS mAbs, b3 and b6, which compete with b12 (Roben et al., J. Virol. 68:4821-4828 (1994)), but do not neutralize HIV-1 or neutralize only tissue culture-line adapted (TCLA) strains and a limited number of atypical primary isolates grown in PBMCs, can also be used.

TABLE 6
Monoclonal Antibodies

| | | HIV-1 | |
|---|---|---|---|
| | | Neutralizing | |
| Antibody | Epitope* | ability? | Reference |
| F105 | CD4BS | No | Posner et al., J. Acquir. |
| | | | Immune Defic. |
| | | | Syndr. 6: 7-14 (1993) |
| b3 | CD4BS | No | Roben et al., J. Virol. |
| | | | 68: 4821-4828 (1994) |
| b6 | CD4BS | No | Roben et al., J. Virol. |
| | | | 68: 4821-4828 (1994) |
| b12 | CD4BS | Yes | Saphire et al., Science |
| | | | 293: 1155-1159 (2001) |
| X5 | CD4i | Yes | Moulard et al., Proc. Natl. |
| | | | Acad. Sci. USA 99: 6913-6918 |
| | | | (2002) |
| 447-52D | V3 Loop | Yes | Gorny et al., J. Immunol. |
| | | | 150: 635-643 (1993) |
| 2G12*** | Outside | Yes | Trkola et al., J. Virol. |
| | CD4BS | | 69: 6609-6617 (1995) |

*CD4BS: CD4-binding site; CD4i: CD4-induced co-receptor binding site;
***Binds carbohydrate

The b3, b6, and b12 human mAbs were obtained from The Scripps Research Institute (San Diego, Calif.). All three antibodies recognized discontinuous epitopes that overlap the highly conserved CD4BS (Roben et al., J. Virol. 68:4821-8 (1994); Pantophlet et al., J. Virol. 77:642-58 (2003)). In this study, the IgG1 form of b12 (“IgG1b12”) and the Fab form of b3 (“Fab b3”) and b6 (“Fab b6”) were used in all experiments involving dot blots and immunoprecipitation. In the surface plasmon resonance analysis, the IgG1 forms of b6 (“IgG1b6”) and b12 (“IgG1b12”) were used in parallel to IgG1b12. Human mAb 2G12, which recognizes a mannose-dependent epitope (Scanlan et al., J. Virol. 76:7306-21 (2002); Trkola et al., J. Virol. 70:1100-8 (1996)), was obtained from POLYMUN Scientific, Vienna, Austria. These mAbs were used to characterize the antigenic and structural diversity of parental gp120 polypeptides and gp120 polypeptide variants and to identify properly folded gp120 polypeptide variants. Although antigenicity of a protein is not necessarily predictive of its immunogenicity, the presence of the above epitopes in gp120 polypeptide variants provides reassurance that the relevant epitopes are present.

### B. Dot-Immunoblot Screening Methods

Dot-immunoblot methods for detecting antibody reactivity of the recombinant parental and shuffled gp120 proteins were used initially to evaluate the results of DNA shuffling of gp120 genes. The concentrations of the plasmids comprising nucleic acids encoding wild-type HIV-1 gp120 polypeptides or polypeptide variants of the invention were determined using a 96-well-based dot-immunoblot method automated by use of a liquid handling system (Tecan). Such plasmids were used to transiently transfect CHO-K1 cells in a high-throughput 96-well format to express the wild-type gp120 polypeptides and shuffled chimeric gp120 polypeptides in both core and full-length forms. If desired, transfection can be automated using the Biomek® FX Laboratory Automation Workstation (Beckman Coulter).

For library screening, CHO-K1 cells (ATCC) were seeded 24 hours before transfection into the first 11 columns of 96-well tissue culture-treated microtiter plates (Falcon) at a concentration of 4×10⁴ cells/Il in 100 μl of serum-containing medium [DMEI/F12 medium (Invitrogen), 10% fetal bovine serum (Hyclone) and 1% 100× penicillin and streptomycin stock (Invitrogen)]/well. After incubation at 37° C. in a humidified incubator with 5% CO₂ for 20-24 hours, 200 nanograms (ng) of each plasmid DNA expression vector (i.e., DNA vector comprising a gp120- or gp120 core polypeptide-encoding nucleic acid of interest) were transfected into a designated well using Lipofectamine 2000 transfection reagent (Invitrogen) according to the manufacturer's instructions. As a control, 200 ng of a plasmid DNA expression vector encoding HIV-1 JRCSF gp120 polypeptide were transfected into a separate well in the same plate. Transfected cells were incubated for another 24 hr and then washed with 200 μl of serum-free OptiMEM I (Invitrogen). Transfected cells were then covered with 200 μl of serum-free OptiMEM I and incubated at 37° C. for three days to allow for protein production/secretion. At the end of the incubation, plates were centrifuged at 2000 rpm for 10 minutes (min) at room temperature and supernatants were harvested.

Supernatants from the transfected cells were analyzed using the dot-blot method to assess the ability of the expressed wild-type and shuffled gp120 molecules to bind to certain anti-gp120 mAbs, such as those shown in Table 6.

Dot-blotting was carried out at neutral pH to maximally retain the natural conformation of the gp120 antigen in solution. Nitrocellulose membranes and a 96-well Minifold® Dot-Blot System were obtained from Schleicher & Schuell. Briefly, cell culture supernatants were suitably diluted and applied to the membranes (e.g., by a Tecan robot) to prepare multiple replicates of the dot-blot membranes for analysis with multiple antibody reagents. The membranes were removed from the dot-blot apparatus and blocked with 5% non-fat milk in PBS-Tween. The membranes were then reacted with mAbs at dilutions ranging from, e.g., 0.06-0.3 μg/ml of mAb in blocking buffer, washed, and reacted with the appropriate secondary antibody conjugated with horseradish peroxidase. Antibody binding was visualized using ECL Plus™ (Amersham Biosciences) followed by exposure to Kodak XAR films as described in greater detail below.

1. Single-Point Dilution Dot-Blot Method.

For each monoclonal antibody (mAb) to be detected, the following procedure was used. 96-well V-bottom plates (Costar) having 12 columns labeled 1-12 and 8 rows labeled A-H were used. On each 96-well dot-blot, supernatants from the transfection of 80 individual chimeric genes were applied in the 8 rows of columns 1 to 10. Column 11 contained supernatant from transient transfections of control DNAs, including the prototype parental gp120 JRCSF, which is defined as the 100% standard. In column 12, a 1:2 dilution of gp120 JRCSF expressed and purified from CHO cells in PBS (Invitrogen) was made from wells A12 to H12 so that the starting protein concentration of A12 was 100 ng in 200 μl of PBS. This protein served both as a control for each dot blot and as an indicator for the exposure time. Forty μl of each transfection supernatant was then diluted into 160 μl of PBS in the same V-bottom plate. The liquids in all wells were transferred and blotted onto a presoaked Qptitran membrane (Schleicher & Schull) using a Minifold® I Dot-Blot System (Schleicher & Schull) in accordance with the manufacturer's recommendations. The membranes were removed from the dot-blot apparatus and blocked with 100 ml of 5% non-fat milk in PBS-Tween (PBS+0.5% Tween 20) at room temperature for 1 hr and the 4° C. for overnight. The immunodetection procedure is described in detail in the brochure for ECL Plus Western blotting detection reagents (Amersham Biosciences, RPN2132). Briefly, each membrane was washed with PBS-Tween, reacted with 4 μg of a human monoclonal antibody diluted in 16 ml of blocking buffer, washed with PBS-Tween, and reacted with a secondary anti-human IgG antibody conjugated with horseradish peroxidase (Vector). After washing with PBS-Tween, antibody binding was visualized with ECL Plus™ (Amersham Biosciences) and the membranes were then exposed to Kodak XAR films. Each blot was exposed multiple times to achieve a signal for quantitation that was within the linear range of the film, using the protein controls in column 12 as a guide.

Images from the exposed films were then quantitated using Phoretix Array v2.00 image analysis software (Nonlinear Dynamics Ltd.). The binding activity of the polypeptide variants with each mAb was normalized to that of the internal gp120 JRCSF control set to 100%.

2. Eight-Point Serial Dilution Dot-Blot Method.

CHO-K1 cells at a concentration of 2.4×10⁵ cells/ml in serum-containing medium were seeded at 0.5 ml per well into a 24-well tissue-culture-treated plate (Costar). After an incubation at 37° C. in a humidified incubator with 5% CO₂ for 20-24 hr, 1.0 μg of plasmid DNA expression vector comprising a gp120 polypeptide-encoding nucleic acid of interest (e.g., one of the recombinant gp120 polypeptide variants of the invention) was transfected into a designated well using Lipofectamine 2000 transfection reagent.

Transfection for each gp120 variant was performed in triplicate. As a control, triplicate transfection of 1.0 μg of expression vector comprising the prototype HIV-1 JRCSF gp120 gene was also performed in three separate wells in the same plate. Transfected cells were incubated for another 24 hr, followed by one wash with 1 ml of serum-free OptiMEM I. Cells were then covered with the same amount of the serum free OptiMEM I and incubated at 37° C. for three days to allow for protein production. At the end of the incubation, the plates were centrifuged at 3,000 rpm for 10 min at room temperature and supernatants were harvested.

In column 12 of a clean 96-well V-bottom plate, a serial 8-point 1:2 dilution of gp120 JRCSF expressed and purified from CHO cells was made in PBS from wells labeled A12 to H12 so that the starting protein concentration of A12 was 100 ng in 200 μl of PBS. This protein served both as a control for each dot blot and as an indicator for the exposure time. Columns 1 to 11 were used for making a 8-point 1:2 serial dilutions of transfected supernatants in PBS, from well A to well H of each column. The starting volume of supernatants in well A was 200 μl. The liquids in all wells were transferred and blotted onto a presoaked Qptitran membrane (Schleicher & Schull) using a Minifold® I Dot-Blot System (Schleicher & Schull) in accordance with the manufacturer's recommendation. This procedure was carried out for each mAb to be detected.

The immunodetection procedures and image analysis procedures used for the 8-point serial dilution dot-blot method were the same as those used for the single-point dilution dot-blot method.

### C. Characterization of Parental gp120 Polypeptides

We transfected plasmids expressing each of the ten parental gp120 genes into CHO-K1 cells and analyzed the resulting supernatants by a dot-blot immunostaining procedure for their respective abilities to bind an anti-His-tag mAb, a polyclonal serum against HIV-1 IIIB gp120, and the human mAbs b3, b6, b12 and 2G12 (Trkola, A. et al., J. Virol. 70:1100-1108 (1996); Scanlan, C. N. et al., J. Virol. 76:7306-7321 (2002)). Analysis of the full-length gp120 and gp120 core parental constructs showed that all of the parents except 92HT593 and 92US712 produced secreted proteins that were readily detected with mouse anti-gp120 polyclonal serum, the anti-His-tag mAb and at least one of the four human mAbs (data not shown). The 92HT593 and 92US712 parental sequences were found to have internal stop codons, probably introduced during the PCR amplification steps. The expression levels of the remaining eight parental gp120 proteins were similar as judged by the anti-His-tag signals and their reactivity to 2G12 (data not shown). The differences in binding of the polyclonal anti-Env serum are likely due the sequences differences of up to 32% among these gp120 proteins.

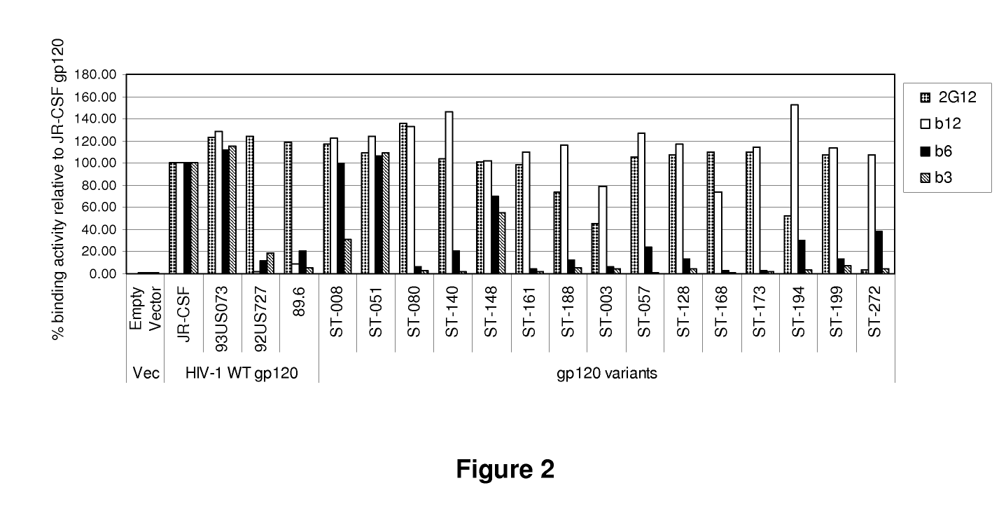

We found that the binding activities and/or affinities of the three anti-CD4BS mAbs (b3, b6 and b12) were typically linked among the wild-type parental full-length gp120 polypeptides. The binding activities and/or affinities between the wild-type gp120 polypeptides and these three mAbs were either approximately equally strong (see JRCSF and 93US073) or approximately equally weak (92US727 and 89.6). See FIG. 2. The gp120 core polypeptide sequence prepared from each respective parental sequence exhibited b12 reactivity that was reduced by at least 8-fold based on a dot-blot analysis. However, the reactivity of b3 and b6 with the gp120 core constructs remained linked.

### D. Characterization of Chimeric gp120 Polypeptide Variants

The 10 parental sequences were used in in vitro DNA recombination procedures to generate two libraries of recombined full-length chimeric gp120 genes that were cloned directly into the expression vector. One library contained all ten starting genes, whereas the second library was generated using only the eight sequences that encoded secreted gp120 (the 92HT593 and 92US712 env genes found to have internal stop codons were excluded). The first step in the screening procedure (Tier 1) involved the analysis of thousands of clones for reactivity with mAb b3, b6, or b12. In this first tier of screening, similar amounts of DNA were transfected into CHO-K1 cells and the supernatants were harvested and screened for binding to b12 and a pool of b3 and b6. The plasmids that produced variants with positive binding to either reagent were selected and retransfected into CHO-K1 cells for a second tier of dot-blot screening using four human mAbs 2G12, b3, b6 and b12. As expected, the library that included the truncated gp120 genes 92HT593 and 92US712 had a low positive rate: only 89 of 2520 clones screened (3.5%) were found to react with at least one mAb. For the library from which 92HT593 and 92US712 were omitted, 160 of 1000 clones (16%) reacted with at least one of these antibodies. We combined clones from both libraries that were positive in the Tier 1 screening and confirmed their reactivity in the Tier 2 screening. All plasmids encoding variants with detectable binding to at least one of the four mAbs were retransformed into E. coli to isolate single colonies. DNAs were prepared, the concentrations normalized, and each was retransfected into CHO-K1 cells in culture to confirm and quantitate binding to the same four human mAbs.

A number of recombinant gp120 polypeptide variants as well as four representative HIV-1 subtype B primary isolate gp120 recombinant constructs were analyzed for their antigenic profiles using the single-point dot-immunoblotting method (FIG. 2).

Compared to the representative wild-type parental HIV-1 gp120 polypeptides, 14 out of 15 of our representative polypeptide variants exhibited novel or unique antigenic profiles with a moderately to significantly weakened binding activity and/or affinity for non-neutralizing mAbs b3 and b6, while exhibiting an increased or at least unchanged binding activity and/or affinity for the most potent neutralizing antibody b12 (FIG. 2). ST-051 had an antigenic profile similar to that of JRCSF. In vitro recombination of HIV-1 gp120 genes thus created multiple examples of novel gp120 proteins with antigenic profiles not found among the proteins encoded by the parental gene sequences.

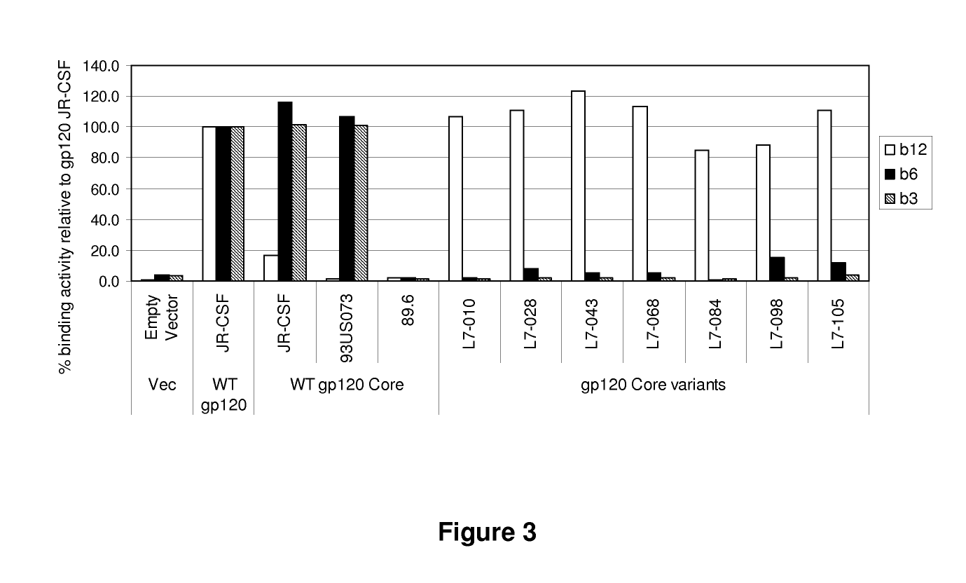

Using the same system, recombinant gp120 core polypeptides constructed from HIV-1 subtype B primary isolates showed reduced b12 binding for the originally b12-positive parents, although their binding to each of b3 and b6 remained linked, as was observed in the corresponding full-length polypeptides (FIG. 3). All 7 representative gp120 core polypeptide variants shown in FIG. 3, however, exhibited a significantly increased binding to b12 when compared to representative wild-type parental HIV-1 gp120 core constructs. The binding activity or affinity of each gp120 core polypeptide variant to each of mAb b3 and b6 was either significantly weakened or completely undetectable in the assay system.

Interaction of seven representative chimeric full-length gp120 polypeptide variants of the invention (ST-008, ST-0051, ST-080, ST-140, ST-148, ST-151, and ST-188) with another human monoclonal neutralizing mAb IgG X5 was also determined using the single-point dilution dot-blot method. While the binding of X5 to HIV-1 wild-type gp120 molecules was detectable for 89.6, JRCSF, and 93US073, only one of the gp120 polypeptide variants tested showed detectable binding to X5 (data not shown). Our results suggested that the binding of a gp120 polypeptide variant to b12 and to X5 could be separated. X5-binding activity was negative for all gp120 core polypeptides tested, including all 7 gp120 core polypeptide variants and 3 gp120 core polypeptide variants constructed from WT HIV-1 isolates (data not shown).

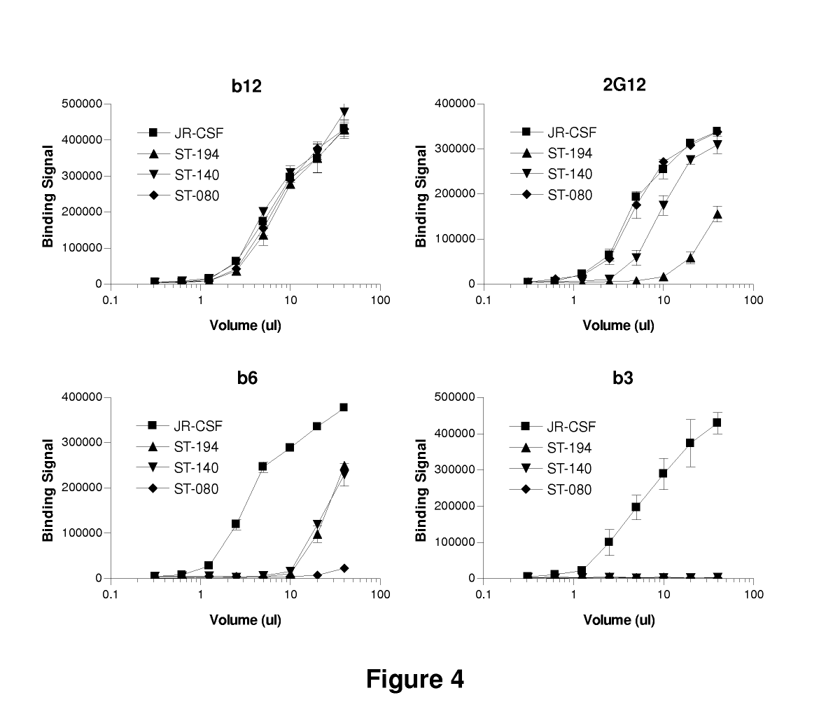

The conditions of the high-throughout dot-blot screening method were chosen to allow for quick and reproducible quantitative estimates of mAb binding with a single dilution of the supernatants from transfected cells. To verify that the novel antigenic properties could be confirmed over a broader range of concentrations using the same dot-blot assay, we selected three b12-binding gp120 full-length polypeptide variants with different degrees of reduction in binding to b3, b6, b12, and 2G12. Transient transfections were performed in triplicate using plasmid DNA vectors encoding the variants ST-080, ST-140, and ST-194 and the JRCSF parent, respectively. An 8-point serial dilution of each supernatant was prepared and four replicate blots were made and reacted with b3, b6, b12, and 2G12. As shown in FIGS. 4A-4D, b12 bound strongly to all three full-length gp120 variants as well as to the JRCSF gp120. The differences of b6 and 2G12 binding were also consistent with the earlier dot-blot results using a single dilution. It is noteworthy that b3 binding was undetectable for all three variant proteins, representing a ≧128-fold reduction in b3-binding activity. The binding of b6 to ST-194 and ST-140 was weakened by about 8-fold and to ST-080 by more than 64-fold.

### E. Immunoprecipitation Analyses

Since the dot-blot prescreening method involved immobilization of antigen to a solid-phase nitrocellulose matrix, we also investigated whether the antibody-binding characteristics of the gp120 variants could be confirmed in a liquid-phase immunoprecipitation assay, which has been successfully used by others to evaluate the antigenicity of modified HIV-1 Envelope (Env) constructs (Yang, X. et al., J. Virol. 76:4634-4642 (2002)).

1. Immunoprecipitation Assays.

Healthy CHO-K1 cells at a concentration of 6×10⁵ cells/ml in serum-containing medium were seeded into 6-well plates, 2 ml per well, and incubated at 37° C. for 20 hr. Plasmids (6 μg) expressing a gp120 full-length or core polypeptide were transfected into a designated well using Lipofectamine 2000 transfection reagent. As a control, a plasmid (6 μg) expressing the corresponding JRCSF gp120 full-length polypeptide or JRCSF gp120 core polypeptide was transfected into a separate well in parallel. Cells were maintained at 37° C. and twenty-four hours after transfection, the cells were washed once with labeling medium containing 90% DMEM without methionine (Invitrogen), 10% fetal bovine serum dialyzed (Invitrogen), 1% 100× L-glutamine (Invitrogen), 1% 100× Sodium Pyruvate (Invitrogen) and 1% 100× penicillin and streptomycin (Invitrogen) for 37° C. for 1 hr. Old medium was then removed and replaced with 1 ml labeling medium+200 microCuries (μCi) of ³⁵5-Met/Cys labeling (GE Healthcare). After 24 hr of incubation, culture supernatants were harvested and stored at −20° C.

For each immunoprecipitation reaction, 30 μl of Protein L-agarose suspension (Genomics One International) was washed once with 300 μl of PBS to remove storage buffer residue. All wash buffers in this and the following steps were removed by a 20 second spin at 5,000 rpm and careful aspiration. Protein L-aragose was resuspended into 300 μl of PBS along with either 4 μg of human mAb or 5 μl of the mouse anti-gp120 polycloncal serum and incubated with slow rotation for 1 hr at 4° C. to allow the formation of an antibody-Protein L-agarose complex. The complex was washed three times with 300 μl of CHO cell serum-containing medium and resuspended into 300 μl of the same medium. After mixing with 300 μl of ³⁵S-Met/Cys-labeled culture supernatant, the mixture was incubated with slow rotation for 2 hr at 4° C., followed by three 10-minute washes with slow rotation with 500 mM NaCl, 0.02% sodium azide, 0.1% Triton X100 in PBS, and one final wash with PBS. After the final spin, the gp120-antibody-Protein L-agarose complex was resuspended into 15 μl of 1×LDS loading buffer (Invitrogen) and separated on a 4-12% SDS-PAGE gel (Invitrogen). After drying, the gel was exposed to a low-energy storage phosphor screen (GE Healthcare) at room temperature overnight. The image on the screen was acquired and quantitated using a Storm PhosphorImager with ImageQuant System (GE Healthcare).

We quantitated the total amount of gp120 polypeptide variant expressed from each construct by precipitating the labeled proteins with a saturating level of a polyclonal anti-gp120. The expression level of each gp120 core polypeptide was estimated using a serial-dilution dot-immunoblotting method with a using a monoclonal anti-His tag antibody (Amersham Biosciences) according to the manufacturer's instructions.

2. Immunoprecipitation Analyses of gp120 Antigens.

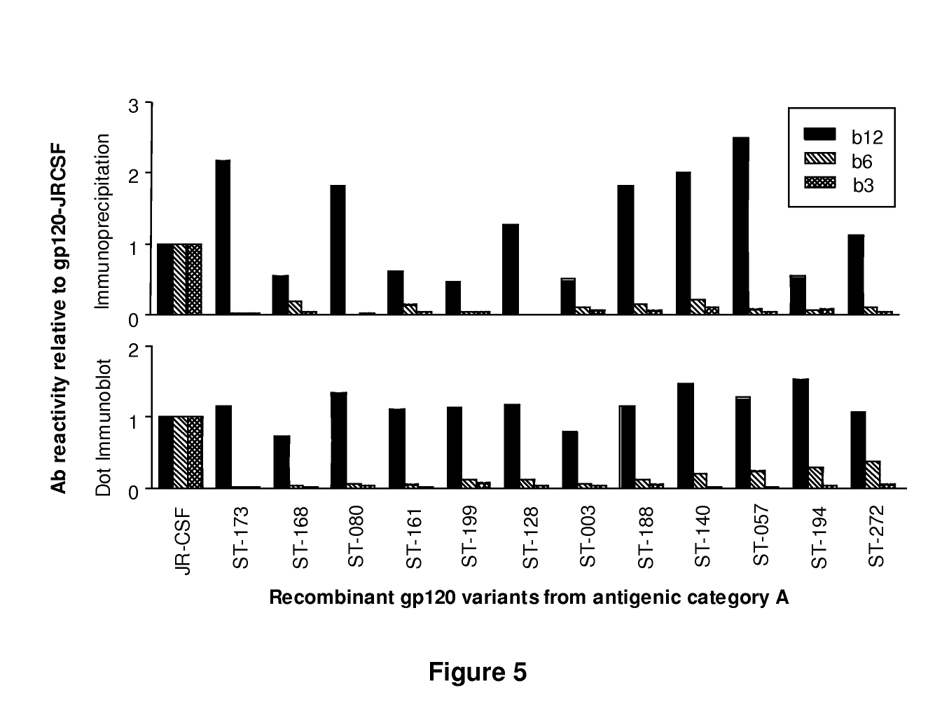

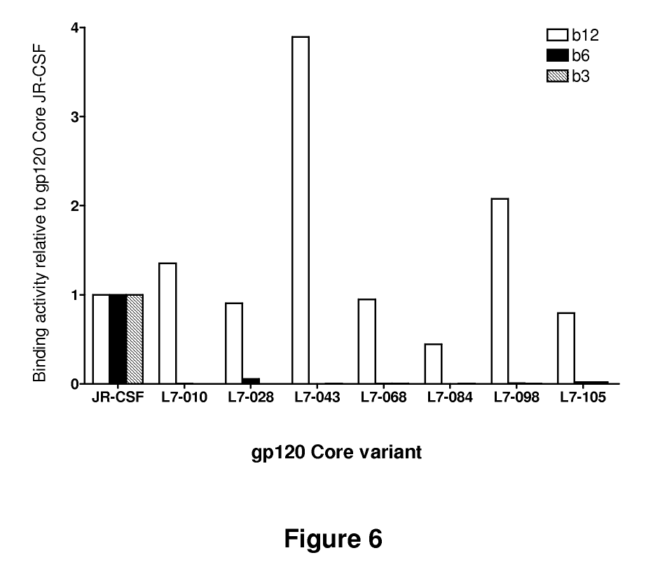

A number of representative gp120 full-length and gp120 core polypeptide variants were analyzed using ³⁵S-Met/Cys labeling followed by immunoprecipitation with Mabs b3, b6, and b12 using the procedure discussed above. Representative gp120 full-length variants included ST-173, ST-168, ST-080, ST-161, ST-199, ST-128, ST-003, ST-188, ST-140, ST-057, ST-194, and ST-272; representative gp120 core polypeptide variants included L7-010, L7-028, L7-043, L7-068, L7-084, L7-098, and L7-105. As discussed below, the gp120 polypeptide precipitated by each of the CD4BS mAbs was quantitated by SDS-gel electrophoresis and normalized to the expression level determined by precipitating the labeled proteins with a saturating level of a polyclonal anti-gp120 antibody. As shown by the normalized data (FIG. 5A), none of the clones was precipitated by the non-neutralizing b3 and b6 mAbs, whereas all of the proteins were precipitated by b12. These immunoprecipitation results indicate that the high-throughput dot-blot assays used for in vitro screening are reflective of the solution-phase antigenicity characteristics of the chimeric variants.

### F. Surface Plasmon Resonance Analyses

After confirming the antigenic profiles of gp120 full-length and core variants with the immunoprecipitation assay discussed above, humanized codon-optimized genes for selected gp120 full-length polypeptide variants (e.g., ST-008, ST-051, ST-080, ST-040, ST-140, ST-148, ST-161, ST-188 and gp120 core variants (e.g., L7-010, L7-028, L7-043, L7-068, L7-084, L7-098, and L7-105) were generated for protein expression. Using the procedures described in Example 3, we generated stable CHO K1 cell lines expressing gp120 full-length or core polypeptide variants and purified the expressed gp120 full-length and core polypeptides.

We then characterized the kinetics of interaction of the wild-type gp120 polypeptides and gp120 polypeptide variants with b12 (neutralizing) and b6 (non-neutralizing) mAbs to investigate the basis for the altered antigenicity observed by the dot-blot and immunoprecipitation assays. Antigen-antibody interaction kinetics were analyzed for representative variants (e.g., ST-080 and ST-140) using a Biacore 2000 system (GE Healthcare) and compared to those of wild-type JRCSF gp120 as follows.

All four flow paths of a CM5 Chip (Biacore/GE Healthcare) were immobilized with 18,000 RU (response units) of goat anti-human gamma chain antibody (KPL, Inc.) by flowing 50 μg/ml of the antibody in acetate buffer (pH 5.0, Biacore/GE Healthcare), at a rate of 10 μl/min. The human mAbs b12 and b6 (100 RU per cycle) were captured on the surfaces of flow paths 2 and 4, respectively, at a flow rate of 10 μl/min. Flow paths 1 and 3 remained unbound to serve as a reference surface for b12- and b6-binding, respectively. The human mAbs b12 and b6 were diluted in Biacore system buffer (HBS-EA buffer, Biacore/GE Healthcare) to a concentration of 0.4 μg/ml. Purified gp120 full-length or gp120 core polypeptides were diluted into HBS-EA buffer at 10 different concentrations: 0, 0.78, 1.56, 3.13, 6.25, 12.5, 25, 50, 100, and 200 nanoMolar (nM). A duplicate dilution was made for 25 nM. This range of concentrations of the gp120 polypeptide (0, 0.78, 1.56, 3.13, 6.25, 12.5, 25, 50, 100 and 200 nM) was injected onto the antibody surface in random order, with 25 nM repeated as one more cycle, at a flow rate of 30 μl/min for 2 min, followed by a 5-min dissociation time. After each association and dissociation cycle, the goat anti-human gamma chain surface was regenerated by a 1 m run followed by 30 s run of 20 mM HCl at a flow rate of 10 μl/m. Then, 100 RU of fresh b12 and b6 were recaptured on the surfaces of flow paths 2 and 4, respectively, for the next cycle. Sensor data were prepared for kinetic analysis by subtracting the binding response collected from the corresponding goat anti-human gamma chain reference surface. The association and dissociation data were fitted simultaneously to a single-site binding with local mass transfer model by using BlAevaluation software from Biacore (GE Healthcare).

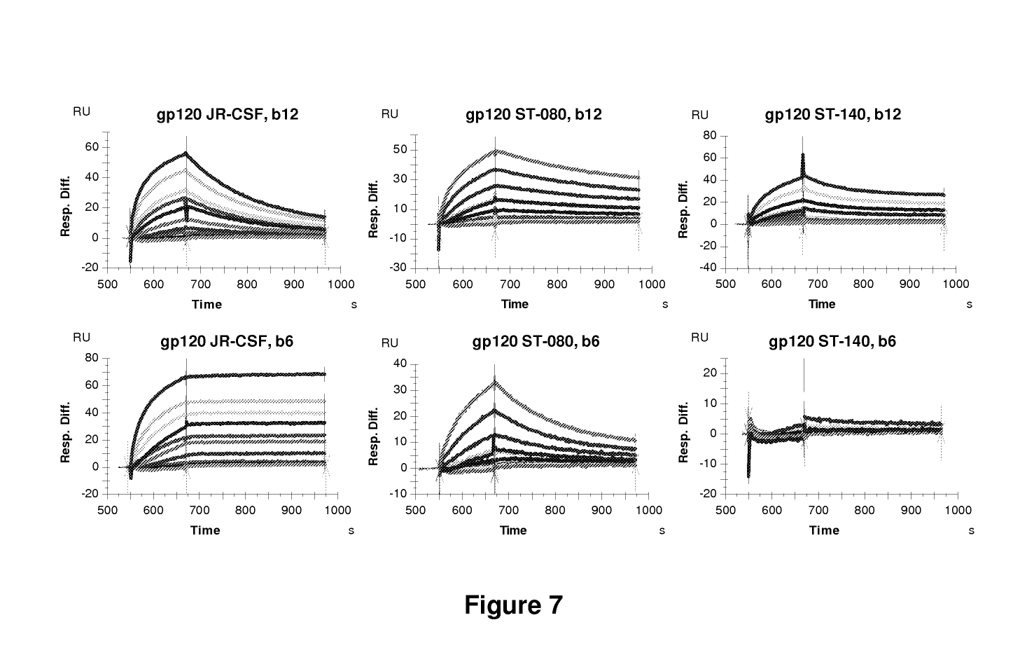

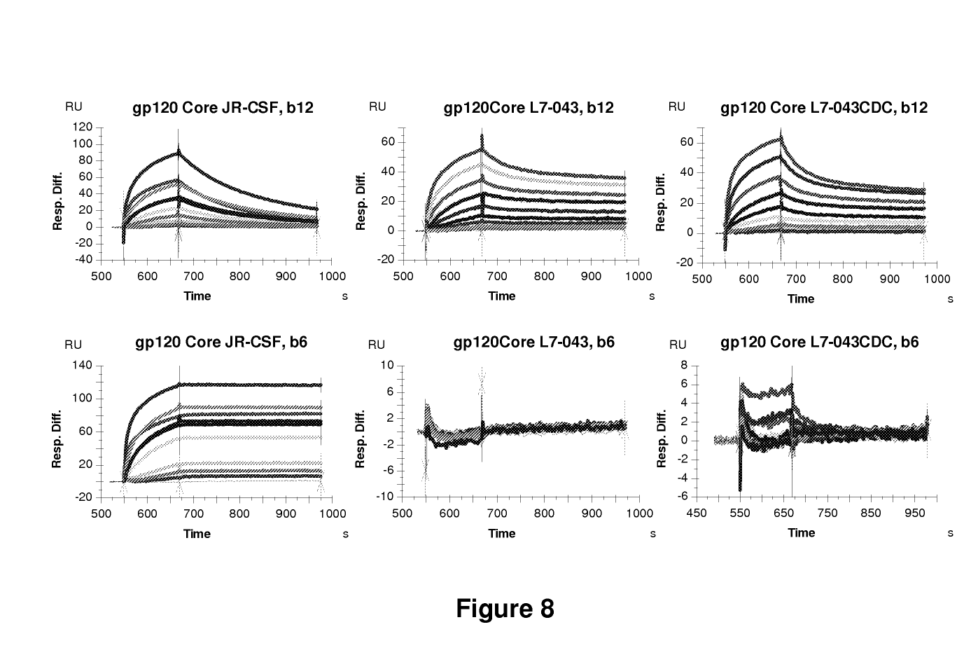

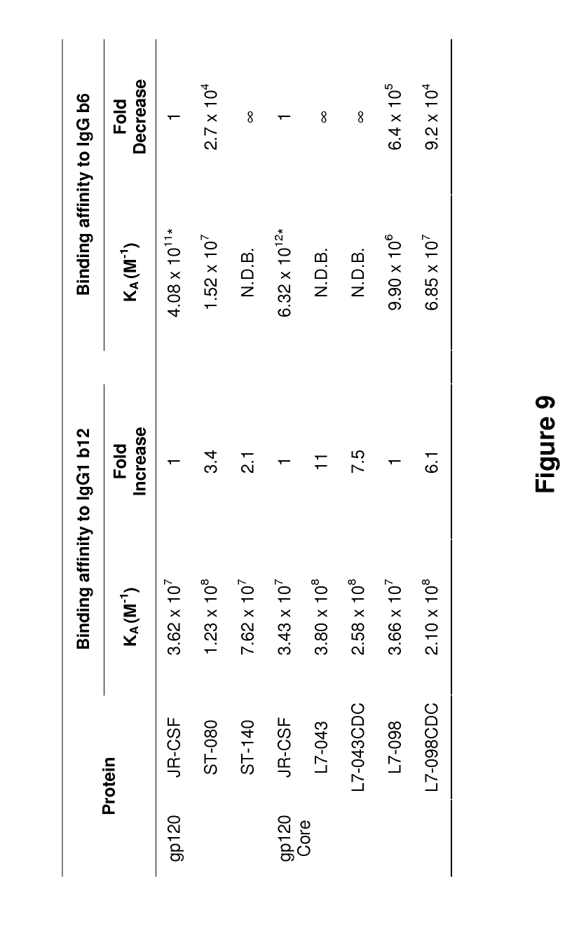

Using the real-time surface plasmon resonance (SPR) technology-based Biacore 2000, we characterized the kinetics of interaction between gp120 full-length or gp120 core polypeptide variants and human mAb IgG1b12 or IgG b6. The sensor data of two representative gp120 full-length polypeptide variants (ST-080 and ST-140) and two representative gp120 core polypeptide variants (L7-043 and L7-043CDC) were compared side-by-side with the gp120 full-length and gp120 core polypeptides of HIV-1 JRCSF, separately (see FIGS. 7 and 8). The quantitative values for K_{A} for HIV-1 JRCSF gp120 full-length and gp120 core polypeptides and the ST-080, ST-040, L7-043, L7-043CDC (with tail), L7-098, and L7-098CDC (with tail) polypeptide variants were determined and are summarized in FIG. 9.

The JRCSF gp120 full-length and JRCSF gp120 core polypeptides bind to b12 with an affinity commonly seen in other antigen-antibody interactions (K_{A}>10⁷). Their binding affinities for b6 were much stronger than their binding affinities for b12, although the magnitude of each difference was not accurately represented due to the extremely slow off-rate that is beyond the detection limit of the Biacore instrument (GE Healthcare) used in the method. Compared to gp120 JRCSF full-length polypeptide, the gp120 ST-080 and gp120 ST-140 full-length polypeptide variants showed a 3.4-fold and a 2.1-fold increase in b12 affinity, respectively, due to a combination of an increased on-rate and a decreased off-rate. In contrast, the affinity of each of the gp120 ST-080 and gp120 ST-140 polypeptide variants for b6 was significantly reduced or completely abolished.

A greater improvement in b12 binding affinity was observed for the representative gp120 core polypeptide variants, similar to our observations from the single-point dilution dot-blot and immunoprecipitation assays. Compared to the gp120 core JRCSF polypeptide, the gp120 core L7-043CDC and gp120 core L7-098CDC polypeptide variants exhibited at least about 7.5-fold and about 6-fold increase in their respective b12 affinities. In contrast, the affinity of each of the gp120 core L7-043CDC and gp120 core L7-098CDC polypeptide variants for b6 was substantially reduced or completely abolished.

In transient transfection experiments, we found that the 32-amino acid CDC tail (SEQ ID NO:22), when present on the C-terminus of a gp120 core polypeptide, generally enhanced expression of such gp120 core polypeptide (data not shown). For certain gp120 core polypeptide variants, such as L7-098, the CDC tail (as shown in L7-098CDC) also increased its mAb binding affinities, likely through improving and stabilizing protein conformation. Comparing the results for L7-098 and L7-098CDC (see FIG. 9), it is seen that the addition of the CDC tail increased the binding affinity of the L7-098 polypeptide variant to b12 by 6-fold and to b6 by 7 fold. L7-098CDC exhibited increased binding affinity relative to the binding affinity of L7-098.

In summary, the novel antigenic phenotypic profiles of the novel gp120 full-length and gp120 core polypeptide variants, which were initially demonstrated with the single-point dilution dot-blot method, have been confirmed by three independent methods including quantitative dot-blot, immunoprecipitation, and Biacore kinetic analyses. The binding activity of a polypeptide for a monoclonal antibody or other ligand or receptor, as observed in the dot-blot and immunoprecipitation assays, is reflective of the binding affinity of the polypeptide for the antibody, ligand, or receptor. A change in the binding activity of a polypeptide, as observed in these assays, is also reflective of a change in the binding affinity of the polypeptide.

Chimeric gp120 variants from the recombinant libraries were identified with a diverse range of novel antigenic phenotypes defined by the extent of binding of broadly neutralizing and non-neutralizing human monoclonal antibodies. Chimeric gp120 variants having a range of antigenic phenotypes were selected for rabbit immunization studies and neutralization assays as described below.

### Example 5

This example describes methods for immunization of rabbits with gp120 nucleic acid and protein constructs of the invention and methods for purifying immunoglobulins (IgG) in serum of rabbits induced by such immunizations.

### A. Methods for Rabbit Immunizations

Electroporation-facilitated DNA-based immunization and protein boosting methods were used to screen wild-type parental gp120 polypeptides and chimeric gp120 full-length polypeptide variants and gp120 core polypeptide variants. Rabbits were chosen for the present studies because rabbit antibodies often contain heavy-chain CDR3 loops equal to or longer than 18 amino acids, similar to the CDR3 loops of existing HIV-1 broadly neutralizing human mAbs. For example, the b12 mAb has an 18 amino acid sequence that rises 15 Å above the surface of the antigen-binding site forming an unique extended finger-like loop. Saphire, E. O., et al., Science 293:1155-1159 (2001). Indeed, Barbas et al. showed that nearly all members of a panel of 32 antibodies against the CD4-binding site developed from phage display have a V(H) CDR3 length of 18 to 22 residues (Barbas, C. F., et al., J. Mol. Biol. 230:812-823 (1993)), which is in marked contrast to the range seen in other human antibodies (Wu, T. T., et al., Proteins 16:1-7 (1993)). The CDR3 loops for the two most potent anti-gp41 neutralizing antibodies are also unusually long: 22 amino acids for 2F5 and 18 amino acids for 4E10 (Kunert, R., et al., AIDS Res. Hum. Retroviruses 20:755-762 (2004). The heavy-chain CDR3 seems to enable neutralizing antibodies to penetrate otherwise occluded regions and bind to their respective epitopes. Id. Wu et al. have shown that CDR3 sequences of this length are very rare in mice (0.2%), but much more frequent in rabbits (8.6%) and humans (15%) (Wu, T. T., et al., Proteins 16:1-7 (1993)).

Rabbits can be immunized with gp120 protein-encoding nucleic acids using other standard delivery procedures, including by needle, injection, or gene gun administration. With electroporation, it is possible to achieve detectable neutralizing activity even with plasmid DNA; neutralizing activity is especially observed when nucleic acids comprising codons optimized for expression in mammalian cells are used.

Protein boosting after plasmid DNA immunization is an effective way to boost the antibody titers. Recombinant proteins useful for boosting can be made using protein production or expression procedures known in the art for each of the chimeric gp120 variants. A recombinant homologous and/or heterologous gp120 protein can be administered after DNA immunization. A homologous protein boost typically comprises administration of a recombinant gp120 protein comprising a polypeptide sequence that is identical or substantially identical to the polypeptide sequence of the protein expressed by the gp120-encoding nucleic acid sequence included in the plasmid vector used for DNA immunization. A heterologous protein boost typically comprises administration of a recombinant gp120 protein comprising a polypeptide sequence that differs from the polypeptide sequence of the protein expressed by the gp120-encoding nucleic acid sequence included in the plasmid vector used for DNA immunization.

Alternatively, a mixture of an effective amount of each of at least two recombinant gp120 variants can be administered as a protein boost following immunization of rabbits with DNA plasmids and/or one or more protein boosts. The homologous or heterologous recombinant protein used for a protein boost can be adjuvanted (mixed) with a suitable amount of an adjuvant (e.g., alum, AS02, CpG, MPL, QS21, etc.), if desired, to boost antibody titers. Typically, 100 μg of the homologous or heterologous recombinant protein for a protein boost is mixed with a suitable amount of an adjuvant for the injection of one rabbit.

Rabbit Immunization Protocols

Certified parasite-free female New Zealand White rabbits were obtained at 8 weeks of age with an average weight of 1.8 to 2.3 kg. Rabbits were housed at the pathogen-free facility at Aldevron LLC (Fargo, N. Dak.). Selected plasmid constructs expressing gp120 variants were used for mg-scale endotoxin-free DNA preparation (i.e., less than 50 endotoxin units/mg) and subsequent animal immunization at Aldevron LLC using protocols approved by the local Animal Care and Use Committee. For DNA immunization, each rabbit was immunized with one single type of HIV-1 gp120-encoding plasmid expression vector (e.g., pMAmp) followed by a homologous or heterologous gp120 protein boost.

After a one-week acclimation period, each rabbit was immunized with three intramuscular injections of a DNA plasmid encoding a gp120 polypeptide using electroporation on days 0, 28, and 56. Each injection comprised a total of 400 μg of DNA injected bilaterally with 200 μg in each of the rabbit's hind muscles. The same procedure was used for administration of a DNA plasmid encoding a wild-type gp120 full-length polypeptide, wild-type gp120 core polypeptide, gp120 full-length polypeptide variant, or gp120 core polypeptide variant. Standard electroporation procedures were employed (see, e.g., Mathiesen, I., Gene Ther. 6:508-514 (1999)).

On day 84, each rabbit received an intraperitoneal (I.P.) injection of 100 μg of recombinant JRCSF gp120 protein mixed with 20 μl of aluminum hydroxide gel adjuvant (ALHYDROGEL ‘85’ 2%, Brenntag Biostor) in a final volume of 1 ml of endotoxin-free PBS (Sigma). Other adjuvants known to those of skill in the art can also be used. About 5 ml of test bleeds were drawn from the ear vein of each rabbit on day 0 and 2 weeks after the third DNA injection (day 70). Terminal bleeds were collected 2 weeks after protein boosting on day 98.

We performed in vivo screening of numerous chimeric gp120 polypeptide variants and gp120 core polypeptide variants and corresponding parental gp120 polypeptides using the above protocol. Typically, for a particular variant clone, if a homologous gp120 protein was not available, we used a recombinant JRCSF gp120 protein for the protein boost. We typically administered one or two booster injections of either the homologous chimeric gp120 polypeptide variant or a heterologous JRCSF polypeptide to the rabbit. Sera were obtained from each immunized rabbit following immunization. In some cases, serum immunoglobulins were purified from the rabbit sera prior to conducting neutralization activity analyses.

### B. Purification of Serum Immunoglobulins

If desired, mammalian serum immunoglobulins (IgG) can be purified before assessing neutralization activity against HIV-1, whatever neutralization assay format is employed. A variety of standard purification methods can be used, including, e.g., DEAE Affi-Gel Blue (BioRad Laboratories) and Protein-A-based IgG purification method (PROSEP-A, Millipore) following the manufacturer's specific instructions for rabbit IgG. Amphotropic MLV virus serves as a control for specificity and is included in the assay of each serum immunoglobulin sample.

Purified IgGs were resuspended in standard PBS and their concentrations were adjusted to reflect those of the original serum sample. All purified serum IgGs were checked on standard SDS-PAGE gel (Invitrogen) and stained with Simply Blue SafeStain (Invitrogen). Two bands of 50 kDa for IgG heavy chain and 25 kDa for IgG light chain were visualized in such gels.

Two exemplary formats are provided below:

(1) DEAE Affi-Gel Blue Column Protocol. One ml of rabbit serum diluted with 2 ml of rabbit-application buffer was desalted by Econo-Pac 10DG column (BioRad Laboratories) and eluted in 4 ml rabbit-application buffer. The desalted serum was applied to a prewashed and equilibrated DEAE Affi-Gel Blue column. The IgG fraction was then Iluted in 20 ml Iabbit-application buffer. Purified IgG was desalted and concentrated by centrifugation using an Amicon Ultro-15 centrifugal filter device with 30,000 NMWL (Millipore) and the final volume was adjusted to 0.5 ml.

(2) An example of the Protein A-based protocol for serum IgG purification is the PROSEP-A antibody purification kit (Millipore), which is a centrifugation-based approach. One ml of rabbit serum was diluted in 1 ml of Binding Buffer A and applied to pre-equilibrated Protein A column. After centrifugation at 100-150×g for 20 minutes, the column was washed and the bound IgG was eluted with Elution Buffer B2 into a fresh tube containing 1.3 ml Neutralization Buffer. Purified IgG was desalted and concentrated by centrifugation using Amicon Ultro-15 centrifugal filter device with 30,000 NMWL (Millipore) and the final volume was adjusted to 0.5 ml.

### Example 6

This example illustrates the neutralizing activities against a variety of HIV-1 pseudoviruses of immunoglobulins (IgG) purified from rabbit serum obtained after immunization of rabbits with at least one chimeric gp120 full-length polypeptide variant-encoding nucleic acid or chimeric gp120 core polypeptide variant-encoding nucleic acid.

### A. Recombinant HIV Pseudovirus-Based Neutralization Assay

The neutralizing activities against HIV-1 viruses of antibodies induced in rabbits by the administered gp120 WT polypeptides and gp120 polypeptide variants were analyzed on our behalf by Monogram Biosciences, Inc. (South San Francisco, Calif.) using an HIV-1 pseudovirus-based neutralization assay (see, e.g., Richman et al., Proc. Natl. Acad. Sci. USA, 100:4144-4149 (2003); Frost, S. D. et al., J. Virol. 79:6523-6527 (2005)). This assay makes use of various HIV-1 pseudoviruses to assess neutralization activities from sera or purified immunoglobulin (IgG) preparations. The assay utilizes recombinant viruses that carry a luciferase reporter gene and are pseudotyped with HIV-1 gp160 envelope proteins. A pseudovirus can be made that is based on any HIV-1 virus (e.g., JRCSF, Bx08). The degree to which an IgG-containing serum or IgG preparation can inhibit entry into cells by a particular HIV-1 pseudovirus is determined by incubating the serum or IgG preparation with the pseudovirus and measuring the percent inhibition of luciferase expression, which corresponds to inhibition of pseudoviral entry into cells.

Details of an exemplary pseudovirus-based neutralization assay for characterizing neutralizing antibody responses to HIV-1 type infection are provided in Richman et al., Proc. Natl. Acad. Sci. 100(7):4144-4169 (2003). Briefly, HIV-1 full-length gp160 envelope (env) sequences are derived from an HIV-1 infected patient plasma or HIV-1 virus stocks or can represent reference sequences from HIV-1 laboratory strains or primary isolates. For example, in one exemplary format, HIV genomic RNA is isolated from virus stocks or patient plasma using standard procedures known in the art. Id. The corresponding cDNA is synthesized by using known reverse transcription methods and oligonucleotide primers. Id. Full-length gp160 env DNA is amplified by PCR using standard amplification methods and primers.

Expression vectors each expressing a gp160 Env amplified from patient plasmas are constructed. Any suitable expression vector can be used; each gp160 env gene is ligated into the vector such that it is operably linked to a suitable promoter in the vector for expression of the gp160 envelope. See, e.g., Richman et al., supra, FIG. 1. A replication-defective, gp160-envelope-gene-defective HIV genomic vector that includes a firefly luciferase expression cassette inserted within the deleted region of the gp160 env gene and a self-inactivating deletion in the U3 region of the 3′ LTR is constructed by known methods. See, e.g., Richman et al., supra, FIG. 1. Stocks of pseudovirus particles comprising gp160 Env proteins are prepared by co-transfecting HEK293 cell cultures with the luciferase-containing HIV genomic vector and a plasmid that expresses HIV envelope under standard conditions for virus particle production. Recombinant pseudotyped virus particles are recovered from the transfected cell cultures 48 hours after transfection using procedures known in the art. Serial dilutions of sera (or immunoglobulin preparations discussed above) are incubated with individual pseudoviruses containing the HIV-1 virus envelopes for 18 hr at 37° C. The pseudovirus/antibody dilution mixtures are then used to infect U87 cells that express the CD4 receptor and CCR5 and CXCR4 co-receptors. Id. Production of luciferase in the U87 cells is dependent on pseudovirus entry and expression of the virus genome. Pseudovirus infectivity is measured 72 hours after inoculation by measuring the amount of luciferase activity expressed in infected U87 cells. Neutralizing activity is calculated by plotting percent inhibition of luciferase activity (which reflects inhibition of pseudoviral infection of cells) versus antibody concentration (1/dilution) compared with an antibody-negative control. Id. The percent inhibition of pseudoviral infection of cells is calculated as (1−[luciferase activity in the presence of ant-body/luciferase activity in the absence of antibody])×100. Id. Inhibition curves defined by the four-parametric sigmoidal function: f(x)=a −[b/(1+(x/c)d)] are fit to the data by nonlinear least squares and bootstrapping and used to calculate the antibody concentration required to inhibit pseudovirus infection by 50%. Neutralizing titers are expressed as the reciprocal of the dilution of antibody or plasma producing 50% inhibition (IC₅₀) of pseudovirus infectivity. Id. A pseudovirus containing a non-HIV retroviral envelope from the aMLV is included in the assay of each serum or IgG as a negative control. The aMLV Env proteins are able to mediate virus entry in U87 CD4/CCR5/CXCR4 cells but are not inhibited by anti-HIV Env antibodies. An IC50 value is considered positive if the percentage of neutralization was 3-fold greater than that of the aMLV control. Negative sera and sera that fail to reach 50% neutralization are assigned a value of 10, which is the lowest dilution tested for each serum. Neutralizing activity of an IgG preparation or IgG-containing serum against a particular HIV-1 gp160 env-based pseudovirus (for example, a pseudovirus derived from JRCSF gp160 env) is believed to be representative of the neutralizing activity against the corresponding HIV-1 virus.

### B. Neutralization Characteristics of IgG Induced by WT gp120 and Chimeric gp120 Full-Length Polypeptide Variants

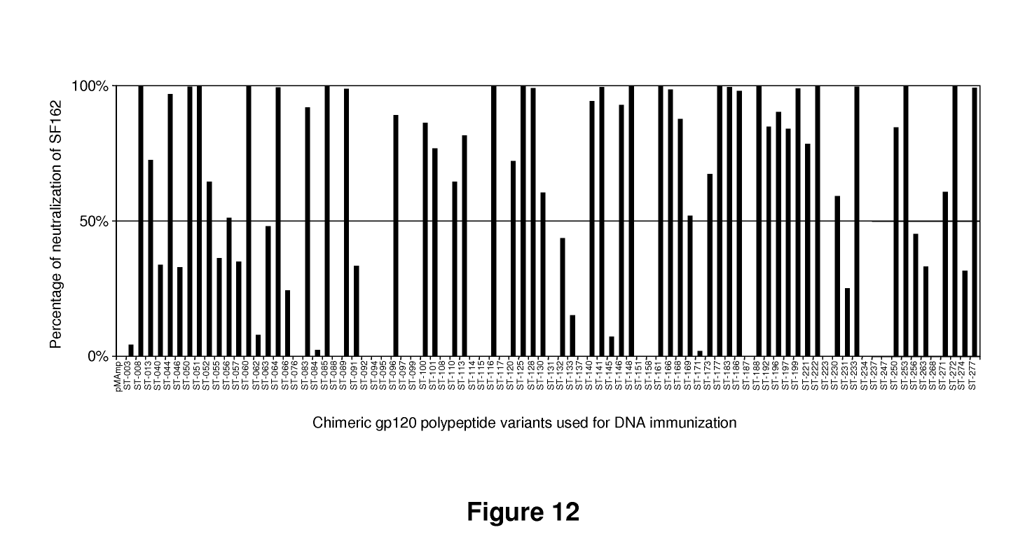

DNA plasmid pMAmp vectors each encoding one of 97 representative chimeric gp120 full-length polypeptide variants were used to immunize rabbits. Two rabbits were immunized with a DNA plasmid encoding each chimeric gp120 polypeptide using the immunization procedures described in Example 5. Briefly, each rabbit received three DNA injections at days 0, 28 and 56 followed at day 84 by one protein boost with purified JRCSF gp120. In some cases, antibodies were purified from sera collected two weeks after the protein boost as described in Example 3 and tested for neutralization activity against the SF162 strain of HIV-1 at the equivalent of a 1 to 7.5 dilution of the original serum volume. The result for the better of the two rabbits for each clone is presented in the histogram shown in FIG. 12. An empty pMAmp vector served as a control.

Out of the 97 variants screened in rabbits (FIG. 12), over 50% showed positive neutralizing activity against the SF162 HIV-1 pseudovirus isolate, demonstrating that these molecules possess structures that are immunologically relevant to HIV-1. This is not surprising, since in vitro DNA recombination creates chimeric genes in which the individual coding segments typically retain their original relative positions in the final reassembled products; the overall primary, secondary and tertiary structures of the chimeric proteins are largely preserved. A high proportion of the novel gp120 variants encoded by the chimeric sequences are functional as determined by antigenic and immunogenic criteria.

To further characterize the neutralization activity of the SF162-positive rabbit sera, we selected a panel of 17 viruses, including eleven from HIV-1 subtype B (Table 7) and six from HIV-1 subtypes A, C, E, F, and G (Table 8). These viruses represent a wide spectrum of sensitivity to a broadly neutralizing human plasma (N16) as well as to the broadly neutralizing human b12 mAb.

Table 7 summarizes the results of pseudovirus-based neutralization assays using eleven representative HIV-1 subtype B (clade B) pseudoviruses and rabbit IgG induced by various wild-type parental gp120 full-length polypeptides and selected chimeric gp120 full-length polypeptides. The subtype B pseudoviruses were: 692, 1196, 92HT594, 93US073, BaL, Bx08, JRCSF, NL4-3, QZ4589, and SF162. The parental gp120 full-length polypeptides were derived from JRCSF, 93US073, 92HT594, 92US657, and 89.6. The chimeric gp120 full-length polypeptide variants were: ST-008, ST-051, ST-148, ST-161, and ST-188. A DNA plasmid encoding each parental gp120 polypeptide or chimeric gp120 polypeptide variant was used to immunize each of two rabbits. Immunizations were performed as described in Example 5, and IgG were purified from the resulting rabbit sera as described in Example 3. The results from both rabbits for each clone are presented in Table 7.

Table 7 shows the percent inhibition of infectivity for each pseudovirus by antibodies induced by each clone at a concentration equivalent to a 1:7.5 dilution of serum. The values correspond to the percent of pseudovirus neutralization compared to the 100% value given by a control sample of the corresponding pseudovirus that contains no immunoglobulin. The results in Table 7 demonstrate that the chimeric gp120 polypeptide variants are able to induce a neutralizing immune response (i.e., a virus-specific neutralizing antibody response) against one or more HIV-1 subtype B pseudoviruses (or viruses).

TABLE 7
Clade B Pseudoviruses Used in Neutralization Assay

| Gp120 | | | | | | | | | | | | |
|---|---|---|---|---|---|---|---|---|---|---|---|---|
| Clone | | | | | | | | | | | | |
| Name | | 692 | 1196 | 92HT594 | 93US073 | BaL | Bx08 | JRCSF | JRFL | NL4-3 | QZ4589 | SF162 |
| Parental | JRCSF | 0% | 22% | 17% | 0% | 71% | 84% | 2% | 0% | 0% | 28% | 100% |
| Clones | JRCSF | 1% | 35% | 0% | 0% | 68% | 84% | 0% | 0% | 0% | 37% | 100% |
| | 93US073 | 0% | 3% | 0% | 0% | 41% | 65% | 0% | 0% | 98% | 13% | 100% |
| | 93US073 | 0% | 0% | 0% | 12% | 60% | 82% | 0% | 0% | 99% | 40% | 100% |
| | 92HT594 | 0% | 0% | 17% | 0% | 0% | 0% | 0% | 0% | 0% | 0% | 98% |
| | 92HT594 | 0% | 0% | 12% | 0% | 0% | 53% | 0% | 0% | 10% | 0% | 86% |
| | 92US657 | 17% | 40% | 8% | 0% | 84% | 92% | 1% | 2% | 100% | 48% | 100% |
| | 92US657 | 21% | 47% | 31% | 0% | 92% | 95% | 12% | 1% | 82% | 52% | 100% |
| | 89.6 | 0% | 0% | 0% | 0% | 0% | 0% | 0% | 0% | 39% | 0% | 82% |
| | 89.6 | 0% | 0% | 15% | 0% | 0% | 67% | 0% | 0% | 70% | 0% | 99% |
| Shuffled | ST-008 | 0% | 73% | 30% | 8% | 90% | 97% | 46% | 6% | 100% | 56% | 100% |
| Clones | ST-008 | 0% | 35% | 15% | 8% | 79% | 96% | 53% | 8% | 99% | 47% | 100% |
| | ST-051 | 0% | 10% | 2% | 4% | 69% | 95% | 38% | 5% | 100% | 28% | 100% |
| | ST-051 | 0% | 0% | 34% | 7% | 55% | 91% | 4% | 0% | 100% | 32% | 100% |
| | ST-148 | 0% | 0% | 18% | 0% | 0% | 43% | 0% | 0% | 16% | 3% | 98% |
| | ST-148 | 11% | 65% | 72% | 14% | 97% | 98% | 12% | 15% | 47% | 75% | 100% |
| | ST-161 | 0% | 30% | 0% | 1% | 68% | 89% | 7% | 2% | 37% | 15% | 100% |
| | ST-161 | 0% | 7% | 36% | 0% | 61% | 86% | 6% | 0% | 64% | 8% | 100% |
| | ST-188 | 6% | 46% | 82% | 6% | 92% | 96% | 7% | 4% | 1% | 63% | 100% |
| | ST-188 | 0% | 0% | 0% | 0% | 0% | 0% | 0% | 5% | 0% | 0% | 0% |

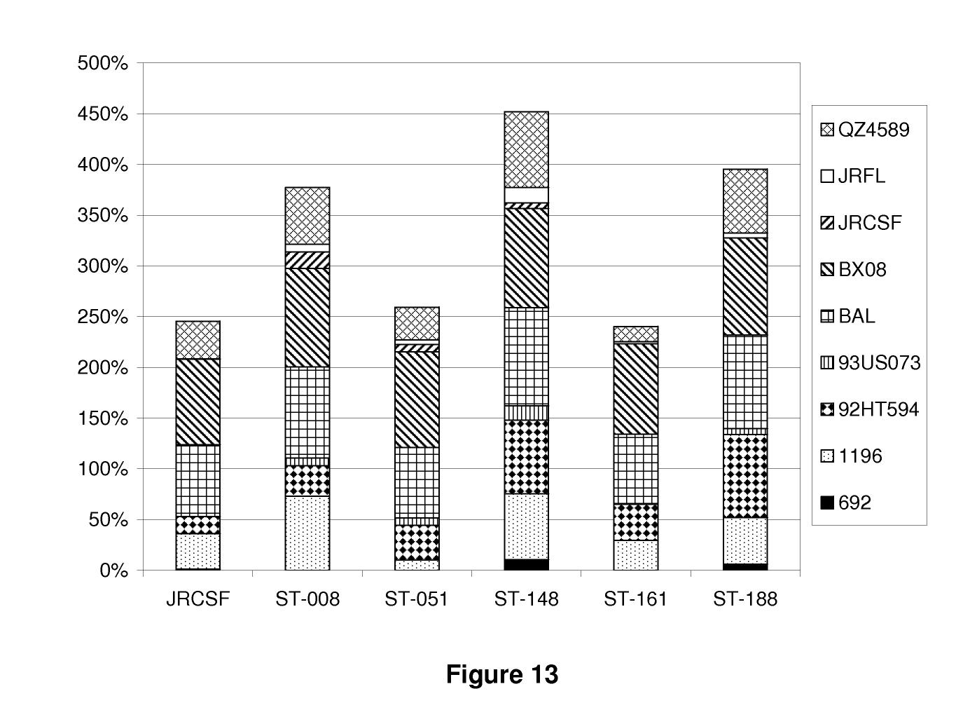

FIG. 13 graphically illustrates the neutralization data for nine of the eleven HIV-1 pseudoviruses in Table 7 in a stacked-column representation. This Figure shows the neutralization (percent inhibition of infectivity) of nine of the eleven HIV-1 pseudoviruses in Table 7. The data relating to SF162 and NL4-3 were omitted from the representation in FIG. 13, since these pseudoviruses are more sensitive to neutralization than the other pseudoviruses and thus tend to obscure the results of the other pseudoviruses. Results are shown for rabbit antibodies induced by a control parental JRCSF gp120 full-length polypeptide and by each of the five representative gp120 full-length variants (ST-008, ST-051, ST-148, ST-161, and ST-188). It is believed these results are representative of the neutralizing activity by each such IgG or serum preparation against the corresponding HIV-1 viruses (e.g., JRCSF, 92HT594, 93US073, etc.). It is believed that these chimeric clones if administered—e.g., as DNA plasmid expressing ST-008 with or without one or more subsequently administered homologous or heterologous protein boosts—to a mammal (e.g., human) would induce antibodies that would neutralize (e.g., inhibit cellular infection of) at least these ten HIV-1 viruses in such mammal to a similar if not equivalent degree.

Table 8 summarizes the results of pseudovirus-based neutralization assays using six HIV-1 non-subtype B (non-clade B) pseudoviruses and rabbit IgG induced by various wild-type parental gp120 full-length polypeptides and selected chimeric gp120 full-length polypeptides. The non-subtype B pseudoviruses were: 92RW020 Rwanda (HIV-1 subtype A); 93BR020Brazil (HIV-1 subtype F); 93BR029Brazil (HIV-1 subtype F); 93MW960 (301960) (HIV-1 subtype C); 93TH305 Thailand (HIV-1 subtype E); and VLGCG4 (HIV-1 subtype G). The parental gp120 full-length polypeptides were derived from JRCSF, 93US073, 92HT594, 92US657, and 89.6. The chimeric gp120 full-length polypeptide variants were: ST-008, ST-051, ST-148, ST-161, and ST-188. A plasmid encoding each parental gp120 polypeptide or chimeric gp120 polypeptide variant was used to immunize each of two rabbits. Immunizations were performed as described in Example 5, and IgG were purified from the resulting rabbit sera as described in Example 3. The results from both rabbits for each clone are presented in Table 8.

Table 8 shows the percent inhibition of infectivity for each pseudovirus by antibodies induced by each clone at a concentration equivalent to a 1:7.5 dilution of serum. The values correspond to the percent of pseudovirus neutralization compared to the 100% value given by a control sample of the corresponding pseudovirus that contains no immunoglobulin. The results in Table 8 demonstrate that the chimeric gp120 polypeptide variants were able to induce an immune response (i.e., a virus-specific neutralizing antibody response) against one or more HIV-1 pseudoviruses (or viruses) of subtypes E, A, C, F, and G. Among the five parental gp120 polypeptides tested, JRCSF gp120 consistently outperformed other parents; the 92US657 gp120 polypeptide also occasionally gave a strong immune response (data not shown). Each of the gp120 polypeptide variants ST-008, ST-051, ST-148, ST-161 and ST-188 had at least one serum that performed as well or better than the JRCSF gp120 polypeptide.

TABLE 8
Non-Clade B Pseudoviruses Used in Neutralization Assay

| gp120 | | | | | | | |
|---|---|---|---|---|---|---|---|
| Clone Name | | 92RW020 | 93BR020 | 93BR029 | 93MW960 | 93TH305 | VLGCG4 |
| Parental | JRCSF | 0% | 0% | 18% | 0% | 14% | 12% |
| Clones | JrCSF | 0% | 0% | 6% | 0% | 3% | 8% |
| | 93US073 | 0% | 0% | 2% | 0% | 2% | 2% |
| | 93US073 | 0% | 0% | 16% | 11% | 12% | 11% |
| | 92HT594 | 2% | 0% | 1% | 4% | 0% | 3% |
| | 92HT594 | 0% | 0% | 13% | 8% | 0% | 0% |
| | 92US657 | 0% | 0% | 26% | 16% | 11% | 42% |
| | 92US657 | 0% | 0% | 35% | 2% | 0% | 31% |
| | 89.6 | 0% | 0% | 0% | 0% | 0% | 0% |
| | 89.6 | 7% | 0% | 0% | 0% | 0% | 2% |
| Shuffled | ST-008 | 2% | 13% | 42% | 20% | 37% | 57% |
| Clones | ST-008 | 2% | 0% | 36% | 11% | 18% | 40% |
| | ST-051 | 24% | 0% | 31% | 9% | 14% | 22% |
| | ST-051 | 0% | 9% | 25% | 15% | 19% | 45% |
| | ST-148 | 2% | 0% | 0% | 0% | 0% | 0% |
| | ST-148 | 0% | 0% | 46% | 11% | 40% | 50% |
| | ST-161 | 0% | 1% | 25% | 11% | 14% | 26% |
| | ST-161 | 0% | 0% | 26% | 11% | 7% | 19% |
| | ST-188 | 6% | 0% | 19% | 0% | 13% | 6% |
| | ST-188 | 0% | 11% | 0% | 2% | 0% | 0% |

To confirm the initial screening results above showing these fives clones—ST-008, ST-051, ST-148, ST-161, and ST-188—are biologically active, we analyzed these five clones in additional rabbits. Table 9 shows the results of neutralization assay involving rabbit sera obtained following immunization of rabbits with one of the five clones using the immunization protocols discussed above. The following Clade B pseudoviruses were tested in the neutralization assay: 692, 1196, 92HT594, 94US073, JRCSF, JRFL, NL4-3, Qz4589, and SF162. Table 9 presents the GMT for each group of rabbits (N=number of animals per group). The data are presented as IC50 values (i.e., the reciprocal of the serum dilution at which 50% neutralization of each indicated virus was obtained). In all experimental groups, the indicated clone was used for DNA injections at Days 0, 28, and 56. All groups were then boosted with gp120 JRCSF at Day 84, and sera were collected at Day 98.

TABLE 9
Clade B Pseudoviruses Used in Neutralization Assay

| gp120 Clone | | | | | | | | | | | |
|---|---|---|---|---|---|---|---|---|---|---|---|
| Name | | GMT* | 692 | 1196 | 92HT594 | 93US073 | JRCSF | JRFL | NL4-3 | QZ4589 | SF162 |
| Parental | JRCSF | GMT | 28 | 60 | 33 | 10 | 40 | 10 | 1514 | 89 | 5063 |
| Clones | | (N) | (6) | (6) | (6) | (6) | (6) | (6) | (6) | (6) | (6) |
| | 93US073 | GMT | 13 | 27 | 13 | 10 | 10 | 10 | 249 | 91 | 2180 |
| | | (N) | (2) | (2) | (2) | (2) | (2) | (2) | (2) | (2) | (2) |
| | 92HT594 | GMT | 26 | 39 | 127 | 11 | 10 | 10 | 270 | 67 | 2087 |
| | | (N) | (2) | (2) | (2) | (2) | (2) | (2) | (2) | (2) | (2) |
| | 92US657 | GMT | 16 | 32 | 18 | 10 | 10 | 10 | 42 | 69 | 3276 |
| | | (N) | (6) | (6) | (6) | (6) | (8) | (6) | (8) | (6) | (6) |
| | 89.6 | GMT | 10 | 33 | 57 | 10 | 10 | 10 | 1558 | 56 | 4988 |
| | | (N) | (2) | (2) | (2) | (2) | (2) | (2) | (2) | (2) | (2) |
| Shuffled | ST008 | GMT | 38 | 71 | 36 | 14 | 54 | 11 | 1656 | 154 | 5558 |
| Clones | | (N) | (6) | (6) | (6) | (6) | (6) | (6) | 4 | (6) | (6) |
| | ST051 | GMT | 17 | 28 | 30 | 10 | 12 | 10 | 1705 | 74 | 2303 |
| | | (N) | (6) | (6) | (6) | (6) | (6) | (6) | (6) | (6) | (6) |
| | ST148 | GMT | 24 | 26 | 116 | 12 | 10 | 11 | NA | 63 | 1837 |
| | | (N) | (6) | (6) | (6) | (6) | (6) | (6) | | (6) | (6) |
| | ST161 | GMT | 17 | 46 | 19 | 10 | 10 | 10 | 50 | 106 | 3908 |
| | | (N) | (6) | (6) | (6) | (6) | (11) | (6) | (11) | (6) | (6) |
| | ST188 | GMT | 14 | 14 | 44 | 11 | 10 | 10 | NA | 20 | 546 |
| | | (N) | (6) | (6) | (6) | (6) | (6) | (6) | | (6) | (6) |

NA: not available

Clone ST-008 was one of the best immunogens in this set of five clones. To further characterize the neutralization activity of the rabbit sera obtained following immunization of rabbits with clone ST-008, we selected a panel of 15 pseudoviruses, including 9 from HIV-1 subtype B and 6 from HIV-1 subtypes A, C, E, F, and G. These pseudoviruses, which are shown in Table 10, represent a wide spectrum of sensitivity to a broadly neutralizing human plasma (N16) as well as to the broadly neutralizing human b12 mAb.

TABLE 10
IC50 Neutralization Titers of Pseudoviruses Used in This Study

| | | GMT | μg/ml | Day 70 GMT | | |
|---|---|---|---|---|---|---|
| Pseudovirus | Subtype | N16 | b12 | JRCSF | ST-008 | P value |
| SF162 | B | 12012 | 0.01 | 1314 | 4211 | 0.010 |
| NL4-3 | B | 2128 | 0.01 | 89 | 198 | 0.041 |
| BaL | B | 1176 | 0.01 | 99 | 172 | 0.056 |
| VLGCG4 | G | 576 | 0.04 | 31 | 50 | 0.032 |
| 1196 | B | 411 | 0.48 | 41 | 69 | 0.058 |
| QZ4589 | B | 365 | 0.03 | 37 | 64 | 0.028 |
| JRCSF | B | 351 | 0.06 | 32 | 101 | 0.038 |
| 93BR020 | F | 298 | 2.08 | 14 | 47 | 0.005 |
| 93MW960 | C | 261 | 0.06 | 14 | 12 | >0.100 |
| 93BR029 | F | 242 | 1.67 | 17 | 27 | 0.039 |
| 6535 | B | 226 | 0.64 | 10 | 41 | 0.001 |
| 92HT594 | B | 147 | 0.02 | 15 | 19 | >0.100 |
| QH0692 | B | 105 | 0.10 | 13 | 20 | 0.009 |
| 93TH305 | E | 87 | 0.71 | 21 | 25 | >0.100 |
| 92RW020 | A | 56 | 1.67 | 16 | 12 | >0.100 |
| AMLV | — | <20 | >2.50 | 10 | 10 | >0.100 |

The ST-008 and JRCSF gp120 clones were then compared in an experiment involving the immunization of 8 rabbits with each clone. As before, three electroporation-mediated DNA injections plus one JRCSF protein boost were carried out. Rabbit sera were collected at two weeks following the third DNA injection (Day 70) and two weeks after the protein boost (Day 98). All sixteen sera were tested for neutralization activity against the complete panel of 15 HIV-1 pseudoviruses shown in Table 10 and the IC50 values were recorded.

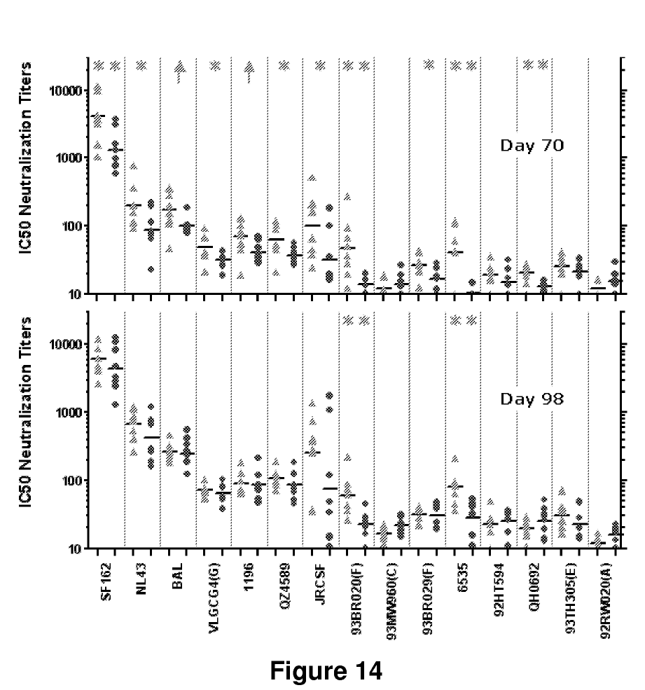

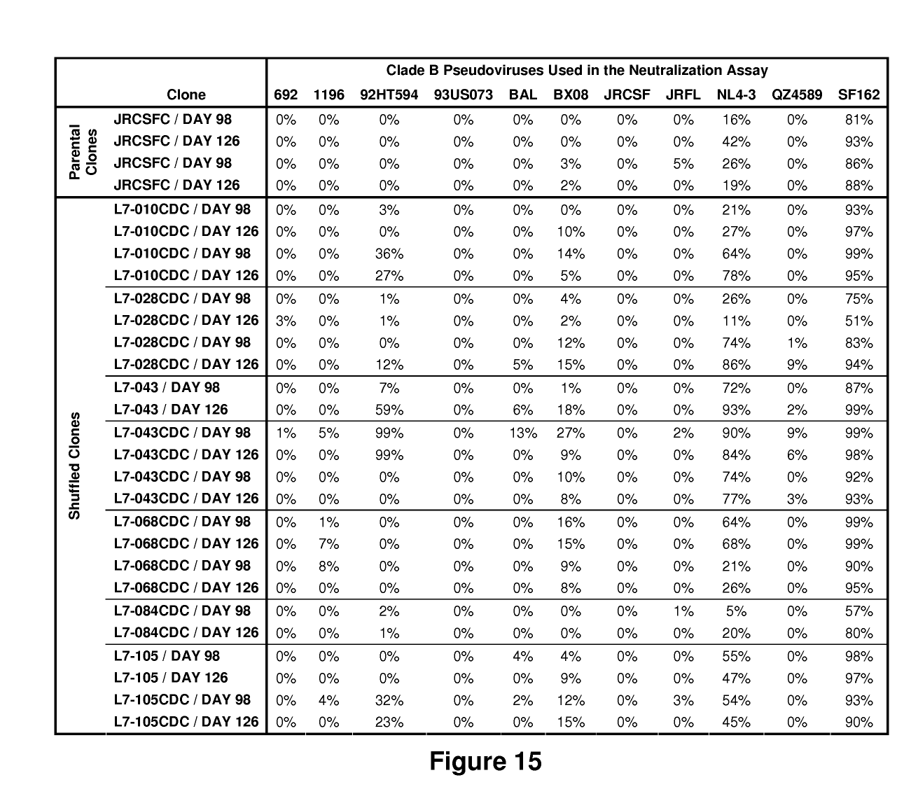

The neutralization activity of the Day 70 sera from JRCSF-immunized rabbits seemed to track the neutralization sensitivity of these pseudoviruses, with exception of the JRCSF pseudovirus, which represents autologous neutralization (FIG. 14). The Day 70 sera of ST-008-immunized rabbits neutralized all nine subtype B viruses and all six non-subtype B pseudoviruses to varying degrees. The ST-008 sera had significantly improved neutralization potency compared to sera induced by JRCSF. We compared the logged IC50 values produced by the sera from each group against individual viruses using a two-tailed, two-sample equal variance Student's t-Test. We found that among the 15 pseudoviruses examined (Table 10), ST-008 was significantly better than JRCSF at inducing neutralizing antibodies for 4 pseudoviruses with P<0.01 and for 5 pseudoviruses with P<0.05. On 2 additional pseudoviruses, the neutralizing activity induced by ST-008 trended higher that those of JRCSF with P<0.10. The superior neutralization activity induced by ST-008 was seen among both the sensitive and more resistant pseudoviruses. The ratio of the Geometric Mean Titer (GMT) values for ST-008 sera versus the JRCSF sera were particularly notable in the cases of the 6535 (3.9-fold), JRCSF (3.2-fold), and 93BR020 (3.3-fold) pseudoviruses; these three pseudoviruses were moderately resistant to neutralization (see Table 10). In no instance was neutralization by JRCSF sera significantly greater than that given by ST-008 sera.

After the protein boost (Day 98 sera), the differences in neutralization induced by ST-008 and JRCSF were less striking (FIG. 14). Nevertheless, the Day 98 sera of ST-008-immunized rabbits still demonstrated significantly improved neutralization potency on two relatively resistant viruses, 6535 and 93BR020 (P=0.004 and 0.003, respectively). In the case of the 92RW020 and 93MW960 pseudoviruses, neutralization by JRCSF sera was significantly better than that by ST-008 sera (P=0.039 and 0.047, respectively).

### C. Neutralization Characteristics of IgG Induced by WT gp120 Core Polypeptides and Chimeric gp120 Core Polypeptides

The nucleic acid sequences encoding selected chimeric gp120 core polypeptides and the parental JRCSF core polypeptide, respectively, were synthesized to introduce codons optimized for mammalian expression, and the corresponding recombinant proteins were prepared as outlined above. Exemplary procedures for synthesizing these high-expressing codon-optimized synthetic gp120 core nucleic acid sequences are presented in Example 7.

We immunized rabbits with a pMAmp DNA plasmid expression vector comprising a codon-optimized nucleotide sequence encoding one of each of representative different chimeric gp120 core polypeptide variants or the WT parental JRCSF gp120 core (shown in FIG. 15 as “JRCSFC”). Two rabbits were used for each immunization protocol with each core variant or parental core clone. Each rabbit received 3 separate injections by electroporation of a plasmid DNA vector comprising a high-expressing codon-optimized synthetic gp120 core gene or the JRCSF gp120 core gene, followed by two separate protein boosts of recombinant gp120 core polypeptide administered by I.P. injection using the immunization protocols described in Example 5. Specifically, the immunization protocol for each rabbit was as follows: On days 0, 28, and 56, a DNA plasmid vector comprising a specific clone was administered by electroporation to each rabbit. On day 84, the first homologous protein boost (adjuvanted with alum as described in Example 5) was administered by I.P. injection. On day 98, each rabbit was bled, serum was collected, antibodies (IgG) were purified from the collected serum, and neutralization assay was conducted using a 1:7.5 dilution of purified antibodies. On day 112, the second homologous protein boost (adjuvanted with alum as in Ex. 4) was administered by I.P. injection. On day 126, each rabbit was subjected to a second bleed, serum was collected, antibodies were purified from the collected serum, and neutralization assay was conducted using a 1:7.5 dilution of antibodies. IgG was purified from each immunized rabbit's serum using Protein A as described previously.

The neutralization activity of the purified rabbit IgG induced by each of the parental JRCSF gp120 core and selected chimeric gp120 core variants against eleven pseudoviruses are summarized in FIG. 15. The percent inhibition of pseudoviral infectivity for each pseudovirus by antibodies induced by each clone is shown.

The WT JRCSF gp120 core clone showed various degrees of inhibition of pseudoviral infection against the TCLA SF162 and NL4-3 pseudoviruses, each of which is easy to neutralize. Each of the selected chimeric gp120 core variants induced antibodies that strongly neutralized the SF162 pseudovirus, as evidenced by the high degree of percent inhibition of pseudoviral infection of cells of the SF162 pseudovirus. For example, chimeric gp120 core variants L7-010CDC, L7-043CDC, L7-068CDC, and L7-105CDC showed the ability to induce antibodies producing at least 90% or more inhibition of SF162 pseudoviral infection at day 98. Each of these chimeric core constructs included the CDC tail (SEQ ID NO:22) described previously. L7-028CDC was able to induce antibodies capable of producing 75% or more inhibition of SF162 pseudoviral infection at day 98 and in one instance 94% inhibition at day 126. Antibodies induced by L7-084CDC also showed significant neutralization activity against the SF162 pseudovirus. Antibodies induced by L7-010CDC, L7-028CDC, L7-043CDC, L7-068CDC, L7-084CDC, and L7-105CDC also showed significant ability to neutralize the NL4-3 pseudovirus. In addition, antibodies induced by L7-010CDC, L7-028CDC, L7-043CDC, L7-084CDC, and L7-105CDC had the ability to neutralize the 92HT594 pseudovirus to varying degrees (as reflected by percent inhibition of pseudoviral infection). Further, antibodies induced by L7-010CDC, L7-028CDC, L7-043CDC, L7-068CDC, and L7-105CDC had the ability to neutralize the BX08 pseudovirus to varying degrees. In contrast, antibodies induced by the JRCSF gp120 core showed no neutralization activity against the 92HT594 pseudovirus and very minimal neutralization activity against the BX08 pseudovirus. As shown in FIG. 15, some of the chimeric polypeptide variants of the invention were also able to induce an immune response against some of the other pseudoviruses.

Antibodies induced by chimeric constructs that did not include the CDC tail (e.g., L7-105 and L7-043) also had ability to neutralize the NL4-3, SF162, 92HT594, and BX08 pseudoviruses to varying degrees. By comparison, constructs that included the CDC tail were typically better able to induce neutralizing antibody responses.

These results demonstrate that chimeric (e.g., shuffled) core forms of the HIV-1 envelope (which do not contain the three variable regions—V1, V2, and V3—that are involved in type-specific neutralization activity) are capable of inducing neutralizing antibodies against one or more HIV-1 pseudoviruses. In particular, these results demonstrate that chimeric gp120 core variants induce neutralizing antibody immune responses against at least two, three, four, or more HIV-1 pseudoviruses, and it is believed these core variants would induce similar immune responses against the corresponding HIV-1 viruses.

### Example 7

This example illustrates procedures for synthesizing nucleic acids encoding polypeptides of the invention, wherein the nucleic acids comprises codons optimized for expression in mammalian cells.

The invention also includes nucleic acids whose codons have been optimized for expression in mammalian cells, such as in, e.g., Chinese hamster ovary (CHO) cells, non-human primate cells, and/or human cells. In one aspect of the invention, synthetic codon-optimized nucleic acids encoding HIV envelope proteins polypeptides of interest or fragments thereof (such as, e.g., gp120 full-length polypeptide variants or gp120 core polypeptide variants of the invention, including, e.g., those exhibiting altered or enhanced binding to HIV neutralizing antibodies and/or HIV non-neutralizing antibodies and/or an ability to induce neutralizing antibodies against one or more HIV-1 viruses) were designed and constructed for improved expression in mammalian cells, such as non-human primate and/or human cells (see, e.g., Haas, J. et al., Curr. Biol. 6:315-324 (1996)). In one aspect, synthetic codon-optimized nucleic acids encoding HIV envelope proteins polypeptides of interest or fragments thereof were constructed for optimum expression in human cells based on nucleotide codons found in highly expressed human genes, as described in Haas et al., supra. Specific codon-optimized nucleotide sequences were determined, e.g., by back translation of a particular gp120 polypeptide sequence of interest using the codon usage table developed by Haas et al., supra.

Because such codon-optimized nucleic acids are effectively expressed in mammalian cells, they are particularly useful for inducing antibodies against HIV viruses in mammals and thus are useful as DNA vaccines and in prophylactic methods for preventing or inhibiting HIV infection in mammals. The codon-optimized nucleic acids can be assembled from oligonucleotides, which can be synthesized using standard nucleic acid synthesis techniques, using standard known protocols and PCR-based methods or purchased from a commercial entity, such as, e.g., Qiagen, Inc. (Valencia, Calif.; Geneart, Toronto, Canada). See, e.g., Stemmer et al., Gene 164:49-53 (1995).

Codon-optimized nucleic acids were characterized with respect to gene expression by transient transfection of a CHO-K1 mammalian cell line with e.g., a DNA plasmid comprising a codon-optimized nucleic acid that encodes a gp120 polypeptide of interest, and the antigenicity of the resultant secreted protein was determined by dot-immunoblotting. The codon-optimized nucleic acids were used for stable transfection of CHO cells; such CHO cells and cell lines thereof can be used for protein production. Such codon-optimized nucleic acids were also used for DNA rabbit immunization by electroporation (discussed in detail above). The invention also includes CHO cells stably transfected with at least one codon-optimized nucleic acid encoding a polypeptide of interest (e.g., a gp120 polypeptide variant or wild-type gp120 polypeptide (core or full-length) and CHO cell lines thereof. Such CHO cells and cell lines are useful for production of such polypeptides.

Alternatively, to obtain a human codon optimized DNA sequence for any amino acid sequence of interest, the amino acid sequence can be back translated to a nucleotide sequence using a standard human codon frequency table (see the codon usage database at the website having the web address kazusa. or .jp/codon).

### Example 8

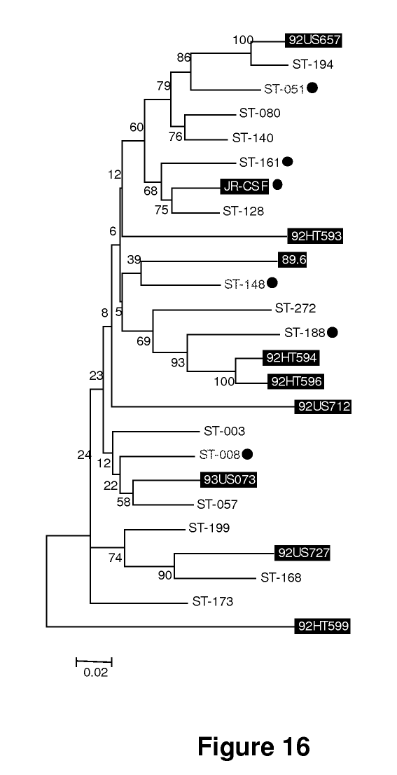

To investigate the genetic relatedness of variants with similar phenotypes, we constructed a phylogenetic tree using the amino acid sequences of 15 chimeric gp120 polypeptide variants and the ten wild-type parental gp120 polypeptides. As shown in FIG. 16, the sequences of the chimeric gp120 polypeptide variants were distributed relatively evenly throughout the sequence space formed by wild-type sequences without any apparent clustering around particular parents. For each of the gp120 variants, we identified the parental sequences from which the individual variants were derived (data not shown). This analysis confirmed the highly chimeric nature of the proteins encoded by the recombined chimeric genes. The gp120 variants showed no apparent similarity to each other and no preference for one parent over the others. Together with the phylogenetic analysis shown in FIG. 16, these results illustrate the ability of in Iitro DNA recombination to give rise to multiple novel solutions (i.e., different sequence variants) for a given antigenic phenotype.

We also examined the relatedness of the most immunogenic variants using the same phylogenic and chimeric analyses. The gp120 variants ST-161 and ST-188 (which were included in the analysis above), along with the three other immunogenic variants ST-008, ST-051 and ST-148, showed no similarity in their phylogenetic relationships and were not related in any obvious way in their pattern of chimerism. These five immunogenic variants also showed several differences in the pattern of binding to the non-neutralizing mAbs b3 and b6 (see FIG. 2). However, these five immunogenic variants and the parent JRCSF all bind strongly to the neutralizing antibodies b12 and 2G12 (FIG. 2).

### Example 9

This example describes the discovery of previously unknown autologous neutralization epitopes on the “silent face” of gp120. We have characterized the relationship between various structural elements of the HIV-1 gp120 envelope glycoprotein and the immunogenicity of this protein. These structure-immunogenicity studies have led to novel observations that provide additional insight into the way the envelope protein can be used as an immunogen in vaccines designed to induce antibodies that can prevent infection by HIV-1.

### A. Immunization of Rabbits with WT JRCSF gp120 Core Induces Autologous Neutralization Activity Against HIV-1_{JRCSF} Which is Independent of Domains V1, V2, and V3

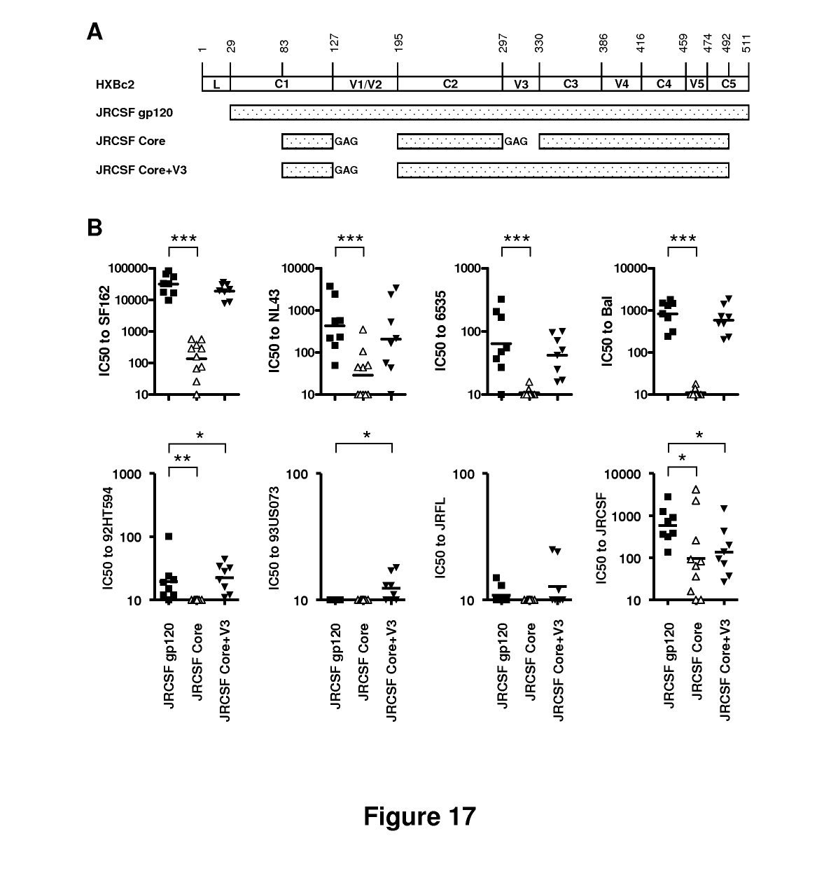

We first investigated the effect of the V3 domain sequences on the immunogenicity of various modified versions of the envelope protein based on the gp120 polypeptide of the JRCSF strain of HIV-1. Using techniques described above, we created three plasmids that expressed: (1) a recombinant polypeptide comprising the entire 504 amino acid polypeptide of gp120 (“JRCSF gp120”, SEQ ID NO:80); (2) a recombinant Core polypeptide form of gp120, lacking parts of C1 and C5 and the variable sequences of V1, V2, and V3 (“JRCSF Core”, SEQ ID NO:90); and (3) a recombinant JRCSF Core gp120 polypeptide with the V3 sequence intact (“JRCSF Core+V3”, SEQ ID NO:100). See FIG. 17A.

We immunized three groups of 8 rabbits with one of the three plasmids at days 0, 28 and 56. A single injection of the recombinant homologous gp120, Core or Core+V3 protein (using AS02A as adjuvant) was given at day 84. Sera were collected at day 98 and analyzed in pseudovirus-based assays to assess the neutralization activity of each serum against a panel of pseudoviruses, as described in Example 6 above. For a number of the pseudoviruses tested (e.g., 6535, 92HT594, BaL, NL43, SF162), the Core immunogen (open triangles), which lacks the V3 sequence, does not induce antibodies that neutralize these viruses as well as the antibodies induced by the full-length gp120 immunogen (filled squares) or the Core+V3 immunogen (filled triangles), both of which contain V3 domain sequences (p<0.05; FIG. 17B). From these results, we conclude that antibodies against the V3 domain of JRCSF are largely responsible for the neutralization of these heterologous (that is, non-JRCSF) viruses.

With respect to autologous neutralization activity (that is, neutralization of the JRCSF virus—or, in this case, the JRCSF pseudovirus—by antisera raised against a JRCSF gp120 polypeptide immunogen), it was found that the JRCSF Core immunogen, which lacks the V3 domain, induced antibodies which neutralize the JRCSF virus. Furthermore, the level of neutralization activity induced by the JRCSF Core immunogen (which lacks V3) was similar to that induced by the JRCSF Core+V3 immunogen (FIG. 17B), suggesting that antibodies against the V3 domain do not contribute to the autologous neutralization activity to any significant extent. The autologous neutralization activity was reduced somewhat in the Core+V3 immunogen compared to the full-length gp120 immunogen (p<0.05; FIG. 17B), suggesting that the V1V2 region and/or parts of C1 or C5 may participate in the neutralization response, or may be involved in a structural configuration resulting in a more potent immunogen. Nevertheless, the fact that the JRCSF gp120 Core immunogen can elicit a strong autologous neutralizing response (which, in some rabbits, approach IC50 values of 5000, FIG. 17B) suggests the presence of autologous neutralization epitope(s) on the Core sequence which are independent of the V1, V2, and V3 domains. It is possible that autologous neutralization epitope(s) are more immunogenic on the Core protein than on the full-length gp120 protein, due to reduced competition among B-cell epitopes in the Core, or due to structural changes that expose autologous epitopes upon the modification of the gp120 molecule.

### B. The JRCSF Autologous Neutralization Epitopes Comprise the V4 and V5 Domain Sequences within the gp120 “Silent Face”

The two remaining variable sequence domains in the gp120 Core, V4 and V5, remain candidates for the autologous neutralization epitope. To investigate this possibility, JRCSF pseudoviruses were constructed with mutations in either the V4 or the V5 domain of gp120 to determine if these pseudoviruses remained sensitive to neutralization by antisera raised against JRCSF immunogens.

FIG. 17B shows that antisera elicited by the JRCSF gp120 immunogen and by the JRCSF Core immunogen both showed strong neutralization activity against a JRCSF Env-containing pseudovirus, but poor to nonexistent neutralization activity against a pseudovirus containing the Env from a closely-related HIV strain, JRFL.

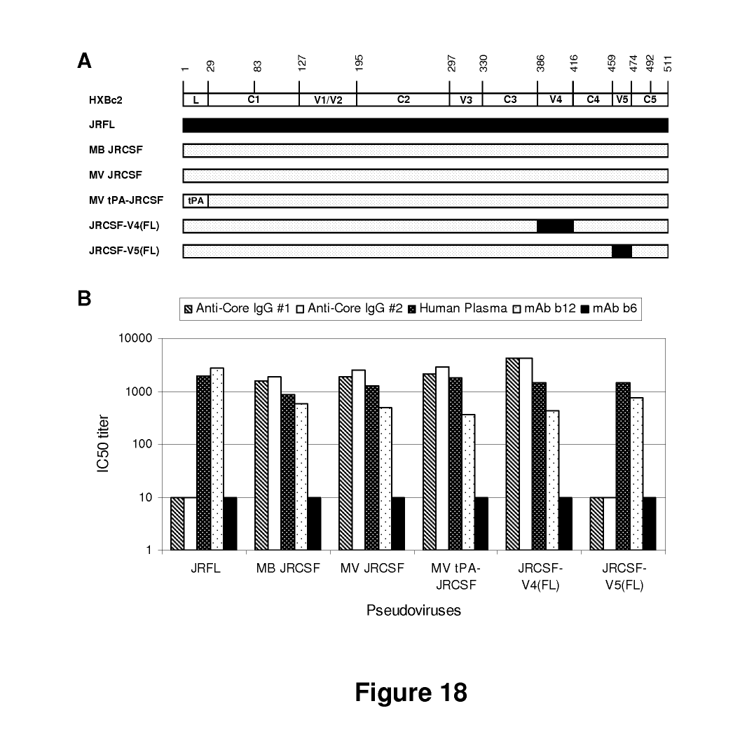

We then generated modified versions of the JRCSF gp160 env gene in which the V4 or V5 sequence of JRCSF was replaced with the heterologous sequence from JRFL (FIG. 18A). These chimeric Env constructs were used to create pseudoviruses for the viral neutralization assays. As controls, we compared the pseudovirus made from the wild-type JRCSF gp160 which is used by Monogram Biosciences (MB-JRCSF) with those from the Maxygen JRCSF gp160 constructs (MV-JRCSF and MV-tPA-JRCSF) to ensure there were no artefacts among the various JRCSF pseudoviral constructs.

IgG preparations were purified from two rabbits immunized with the JRCSF Core immunogen. These IgG preparations strongly neutralized the MB-JRCSF, MV-JRCSF, and MV-tPA-JRCSF Env pseudoviruses to similar extents, whereas essentially no neutralization of the JRFL Env pseudovirus was detected by these IgG preparations (FIG. 18B). Replacing the V4 domain sequence in JRCSF with the V4 domain sequence from JRFL (JRCSF V4 (FL); FIG. 18A) had no negative effect on neutralization by the anti-JRCSF Core IgG preparations—that is, the anti-JRCSF Core IgG preparations strongly neutralized the JRCSF V4 (FL) pseudovirus. However, replacing the V5 domain sequence in JRCSF with the V5 domain sequence from JRFL (JRCSF V5 (FL); FIG. 18A) completely abolished neutralization by the anti-Core IgG preparations.

Importantly, both the JRCSF V4 (FL) and JRCSF V5 (FL) pseudoviruses showed comparable sensitivity towards broadly HIV-1 neutralizing human plasma and the neutralizing mAb b12, but were not neutralized by the non-neutralizing mAb b6. These controls suggest that the heterologous substitutions within the JRCSF Env did not interfere with the overall conformation and neutralization sensitivity of the chimeric Env pseudoviruses.

We further analyzed neutralization of the JRCSF V4(FL) and JRCSF V5(FL) pseudoviruses by a large number of antisera preparations which exhibit autologous JRCSF neutralization activity. We found that approximately 70% of such sera exhibited a 50% or greater reduction in neutralization of the JRCSF V5(FL) pseudovirus compared to the neutralization of the wild-type JRCSF pseudovirus (data not shown), indicating that autologous neutralizing antibodies were most frequently directed against an epitope in the V5 domain.

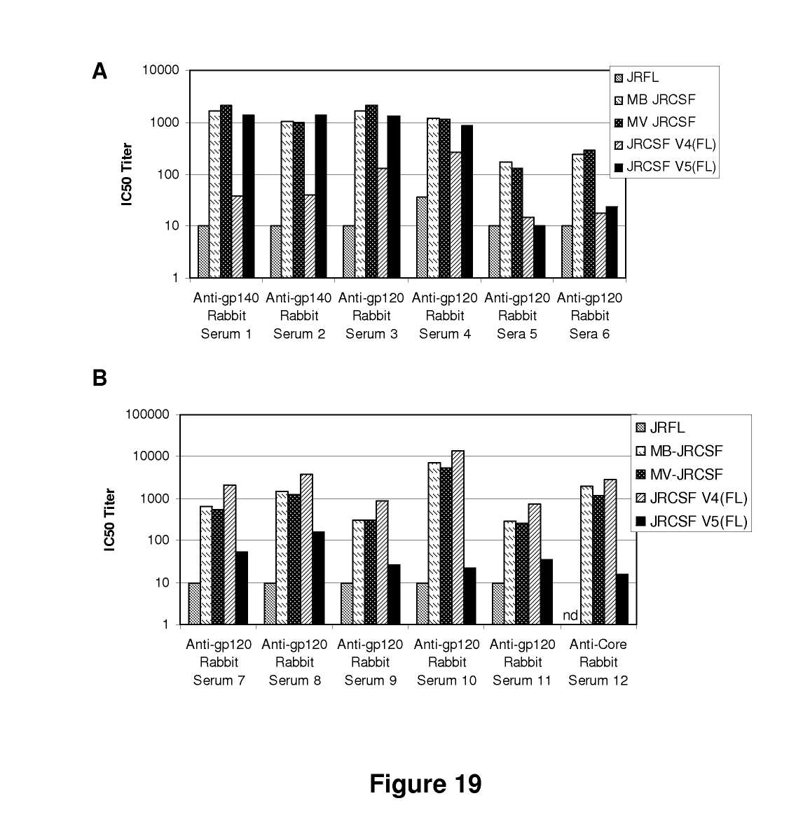

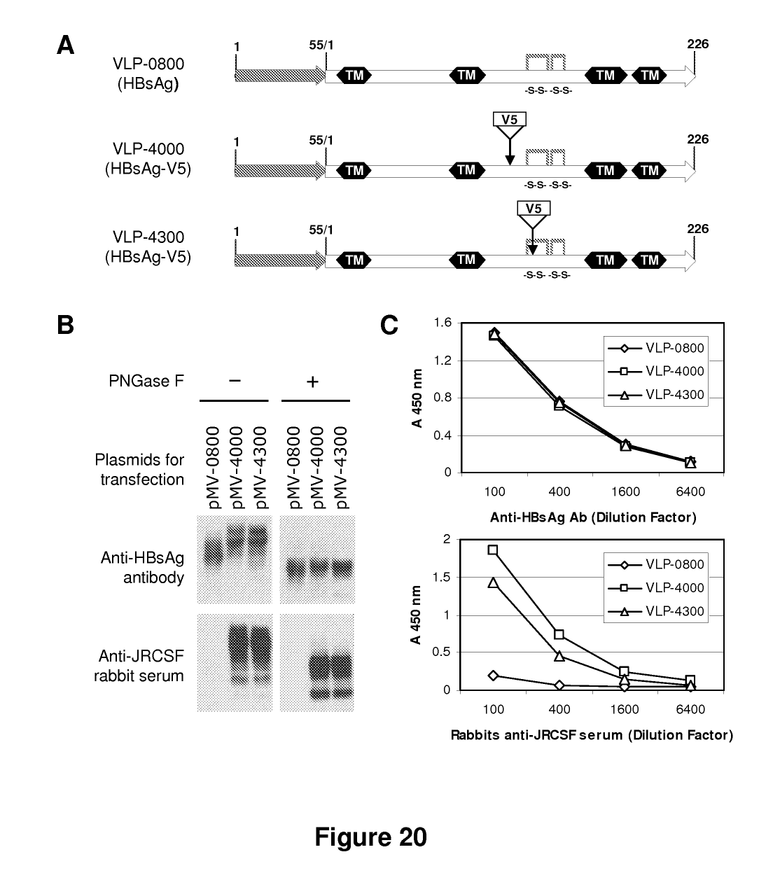

We have also characterized sera which neutralize via epitopes in the V4 domain or in both the V4 and V5 domains. FIG. 19A shows the neutralization activity of serum preparations obtained from rabbits innoculated with the JRCSF Core immunogen. Some antisera (e.g., Sera 1-4) exhibited reduced neutralization activity against the JRCSF V4 (FL) pseudovirus, as evidenced by the large decrease in IC50 titer when such antiserum preparations were assayed against JRCSF V4 (FL) pseudovirus (which contains the JRFL V4 domain and the JRCSF V5 domain), compared to the IC50 titers of these antisera against the JRCSF V5 (FL) pseudovirus (which contains the JRCSF V4 domain and the JRFL V5 domain) and the wild-type JRCSF pseudoviruses (which contain the JRCSF V4 and V5 domains). Furthermore, some of the anti-JRCSF Core sera (e.g., Sera 5-6) exhibit reduced neutralization activity against both the JRCSF V4 (FL) and JRCSF V5 (FL) pseudoviruses (e.g., Sera 5 and 6). Overall, only about 10% of the serum preparations raised against the JRCSF Core immunogen exhibited a 50% or greater reduction in the level of neutralization towards the JRCSF V4 (FL) pseudovirus relative to the level of neutralization of the wild-type JRCSF pseudoviruses, and only about 5% of the anti-JRCSF Core serum preparations tested exhibited a 50% or greater reduction in neutralization of both the JRCSF V4 (FL) and JRCSF V5 (FL) pseudoviruses relative to the wild-type JRCSF pseudoviruses. Although autologous neutralization activity against the V4 domain is less common than autologous neutralization activity against the V5 domain, these findings indicate that V4 can participate in the autologous neutralization response, either directly as a target of neutralizing antibodies, or indirectly by affecting Env structure and antibody recognition of the V5 domain epitope.

Alterations in the V4 domain can in some instances influence the extent of neutralization through the V5 domain epitope. Several anti-JRCSF Core serum preparations actually exhibited a 2- to 4-fold enhancement in neutralization against the JRCSF V4 (FL) pseudovirus relative to the level of neutralization of the wild-type JRCSF pseudoviruses (FIG. 19B). About 30% of the antiserum preparations which exhibit autologous neutralization activity directed against the JRCSF V5 domain (as evidenced by the pronounced decrease in neutralization titer against the JRCSF V5 (FL) pseudovirus, which contains the JRCSF V4 domain and the JRFL V5 domain, compared to the neutralization titer against the wild type JRCSF pseudoviruses, which contain the JRCSF V4 and V5 domains), also exhibit a 2-fold or greater increase in IC50 neutralization titers against the JRCSF V4 (FL) pseudovirus compared to the wild-type JRCSF pseudovirus. These results thus suggest that modifications in the V4 domain may alter Env structure and assist neutralization through an autologous epitope in the V5 domain.

### C. Sera with Strong Autologous Neutralization Activity Contains Antibodies which Bind to Isolated V5 Sequences

If the V5 domain is indeed part of an autologous neutralizing epitope, then anti-JRCSF Core serum preparations with strong autologous neutralizing activity should contain antibodies which specifically bind to JRCSF V5 domain sequences. To test this, the JRCSF V5 domain sequence was inserted into two different sites within the exposed loop of the Hepatitis B surface antigen (HbsAg) polypeptide sequence, to create two V5-HBsAg virus like particle (VLP) fusion constructs capable of forming VLPs, denoted VLP-4000 and VLP-4300 (FIG. 20A). Wildtype HBsAg VLPs and VLPs carrying the V5 sequence were partially purified from transient transfection culture supernatants by ultracentrifugation and used to coat plates for ELISA. Based on the binding of a polyclonal antibody to HBsAg, both of the V5-HBsAg constructs form VLp at levels similar to the wt HBsAg (FIG. 20C). Furthermore, antisera with strong autologous JRCSF neutralizing activity isolated from JRCSF Core-immunized rabbits bound strongly to both of the fusion VLPs, suggesting that the V5 sequences were presented on the surface of the VLPs (FIG. 20C). The fusion constructs were transiently expressed in COS-7 cells, separated on SDS-PAGE and analyzed on Western blot again using the autologous neutralizing antisera from the JRCSF Core-immunized rabbits. FIG. 20B shows that the antisera reacted strongly with V5 sequences in the denatured fusion proteins after SDS-PAGE, with and without prior treatment with N-Glycosidase F (PNGase F; FIG. 20B), indicating that the autologous neutralization epitope formed by the V5 domain is a linear peptide epitope.

### D. Mutations in the V5 Domain Significantly Reduce the Immunogenicity of the JRCSF Autologous Neutralization Epitope

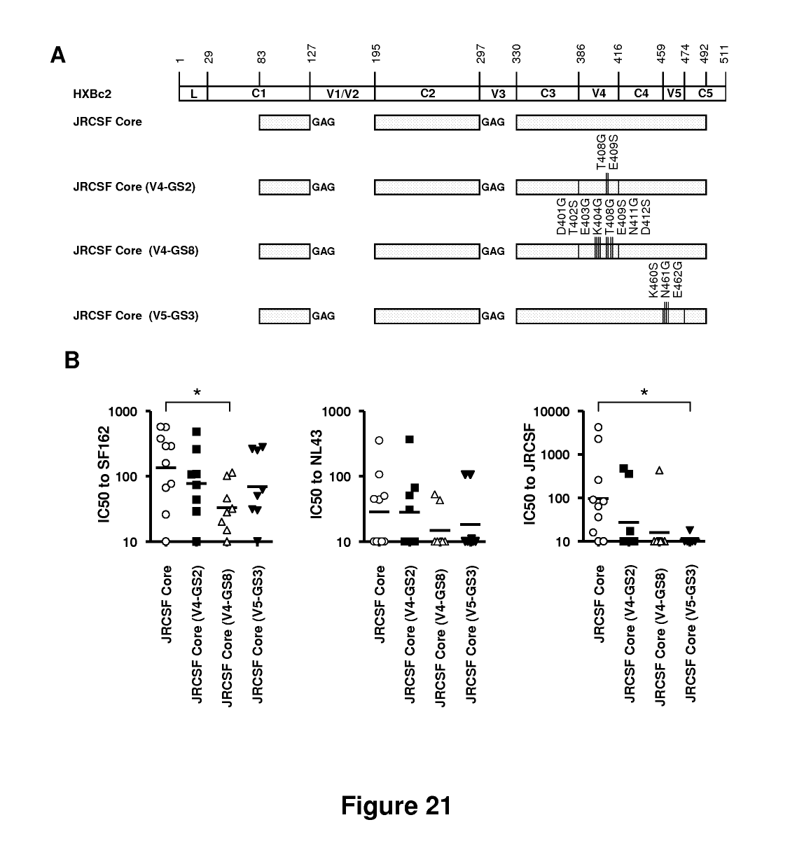

From the experiments described above, both the V4 and V5 domains appear to be involved in autologous neutralization, with V5 playing a dominant role. To determine whether amino acid mutations in V4 or in V5 affect the immunogenicity of the JRCSF autologous neutralization epitope(s), we generated three constructs in which some amino acids in the V4 and V5 domain sequences were replaced with potentially less immunogenic Gly and Ser amino acids as shown in FIG. 21A. JRCSF Core (V4-GS2) (SEQ ID NO:101) has two substitutions in V4, at T408G and D409S; JRCSF Core (V5-GS3) (SEQ ID NO:103) has three substitutions in V5, at K460S, N461G, and D462G; and JRCSF Core (V4-GS8) (SEQ ID NO:102) has eight substitutions in the V4 domain, replacing eight charged or polar amino acids between positions 401 and 412 with Gly or Ser (FIG. 21A). Plasmids encoding the three variants were used to immunize three groups of 8 rabbits each, followed by homologous protein boosting as described in Example 5.

Analysis of the resultant sera showed that sera raised against the JRCSF Core (V5-GS3) immunogen exhibited no neutralization activity against the JRCSF pseudovirus (FIG. 21B). Thus, three amino acid substitutions in V5 was sufficient to abolish the autologous neutralization response against JRCSF (p<0.05), suggesting that the V5 domain is critical to the immunogenicity of the JRCSF autologous neutralization epitope.

Mutations in V4 also negatively influenced the immunogenicity of the JRCSF autologous epitope but were not able to completely abolish the autologous response against JRCSF (FIG. 21B), suggesting that V4 might be indirectly involved in the neutralization response. Between the two V4 mutants, the immunogenicity of the JRCSF autologous epitope on the JRCSF Core (V4-GS8) immunogen was reduced further than that on JRCSF Core (V4-GS2) immunogen (FIG. 21B), suggesting that fewer amino acid mutations in V4 might favor proper presentation of the JRCSF epitope. However, since the neutralization response generated by the JRCSF Core (V4-GS8) against the heterologous pseudovirus SF162 was also significantly reduced (p<0.05; FIG. 21B), it is reasonable to conclude that mutations in V4 also have a global effect on the gp120 Core immunogen. This conclusion is in agreement with what we observed in FIG. 19B, where replacing the V4 domain of JRCSF with the V4 domain of JRFL could sometimes enhance VS-based neutralization, and further supports our conclusion that the V4 domain plays a role in maintaining the structure of gp120 and serves to enhance the immunogenicity of the V5 domain.

### Example 10

The following example demonstrates that deletion constructs (i.e., fragments) of polypeptide variants of the invention may exhibit improved immunogenicity over that of the parent variant polypeptide.

As described in Examples 4 and 5 above, variant ST-008 was created by DNA shuffling and encodes a gp120 protein. Compared to JRCSF gp120, which is capable of inducing the most potent neutralization responses among more than twenty wild-type gp120 proteins tested, ST-008 introduced even stronger neutralization responses (Examples 5 and 6). To investigate whether such improved immunogenicity was solely based on the apparent immunogenic variable loops, such as the V3, V1 and V2, we prepared the following gp120 deletion constructs based on the full-length JRCSF and ST-008 gp120 sequences, shown schematically in FIG. 22A: (i) gp120 lacking the V3 domain sequence (JRCSF gp120ΔV3, SEQ ID NO:104 and ST-008 gp120ΔV3, SEQ ID NO:107), (ii) gp120 lacking the V1, V2 and V3 domain sequences (JRCSF gp120ΔV1V2V3, SEQ ID NO:105 and ST-008 gp120ΔV1V2V3, SEQ ID NO:108), (iii) gp120 lacking the V1, V2, V3, and part of the C1 and C5 domain sequences (JRCSF Core, SEQ ID NO:90 and ST-008 Core, SEQ ID NO:109), and (iv) Core plus the V1 and V2 domain sequences (JRCSF Core+V1V2, SEQ ID NO:106; ST-008 Core+V1V2, SEQ ID NO:110). Each deletion construct plasmid was used to immunize 8 rabbits as described in Example 5.

The neutralization results obtained using the resultant rabbit antisera showed that all four of the deletion constructs derived from ST-008 induced statistically stronger neutralization responses than did the four JRCSF counterparts (FIG. 22B) against the heterologous (pseudo-)viruses SF162, NL43 and BAL. Our results demonstrate that ST-008 has a more immunogenic core backbone that JRCSF, suggesting that the improved neutralizing responses induced by the ST-008 immunogen were not solely based on immunodominant regions such as the V3 domain sequence but is also indicative of a global improvement in the immunogen structure.

The ST-008 variant protein (SEQ ID NO:1) contains the JRCSF amino acid sequence from V3 through C5 (FIG. 23A), and therefore contains the silent face of JRCSF, including the V5 and V4 domain sequences which (as shown in Example 9) are involved in the JRCSF autologous neutralization epitope. We therefore compared ST-008 and its deletion constructs as well as their JRCSF counterparts for their neutralizing responses against the JRCSF strain of HIV-1.

The neutralization results showed that all ten immunogens induced strong autologous neutralization activity against the JRCSF pseudovirus (FIG. 23B). However, the ST-008 gp120ΔV1V2V3 immunogen (which is similar to the Core immunogen in that it lacks V1, V2 and V3, but, unlike the Core, retains the entire C1 and C5 domain sequences) appears to be a superior immunogen for inducing autologous neutralization activity against the JRCSF pseudovirus, compared to the other four ST-008 immunogen constructs and all five of the JRCSF immunogen constructs.

These results support two conclusions. First, a shuffled variant (such as ST-008 and its deletion constructs shown in FIG. 23B) containing JRCSF sequences can induce autologous neutralization activity against the JRCSF virus. Second, the immunogen that lacks the V1, V2 and V3 sequences but retains C1 and C5 (ST-008 gp120ΔV1V2V3) appears to be a more immunogenic form of ST-008 than the Core form (FIG. 23B). This could be due to the influence of C1 and C5 in creating a more stable protein.

### Example 11

The following example demonstrates that mutations which decrease or abolish glycosylation of the JRCSF V5 epitope further improve the immunogenicity of JRCSF autologous neutralization epitope.

The Env protein of HIV-1 is extensively glycosylated with 18-33 glycosylation sites in gp120 alone (Zhang, M et al. Glycobiology 14(12):1229-1246 (2004)). This post-translational modification is associated with various functional consequences. Numerous studies have shown that changes in glycosylation patterns of Env can impact viral fitness, alter protein folding and conformation (Cole, K. S. et al., J. Virol. 78:1525-1539 (2004); Land, A. et al., Biochimie 83:783-790 (2001); McCaffrey, R. A. et al., J. Virol. 78:3279-3295 (2004); Ohgimoto, S. et al., J. Virol. 72:8365-8370 (1998)), and expose neutralizing epitopes on Env rendering the virus more sensitive to neutralization (Derdeyn, C. A. et al., Science 303:2019-2022 (2004); Koch, M. et al., Virology 313:387-400 (2003); Ly, A., and L. Stamatatos, J. Virol. 74:6769-6776 (2000); McCaffrey, R. A. et al., J. Virol. 78:3279-3295 (2004); Reynard, F. et al., Virology 324:90-102 (2004); Song, B. et al., Virology 322:168-181 (2004); and Wei, X. et al., Nature 422:307-312 (2003)). It has therefore been hypothesized that the greater sensitivity of epitopes to neutralizing antibodies that results from altering glycosylation patterns could also render those epitopes more immunogenic (Reitter, J. N. et al., Nat. Med. 4:679-684 (1998)).

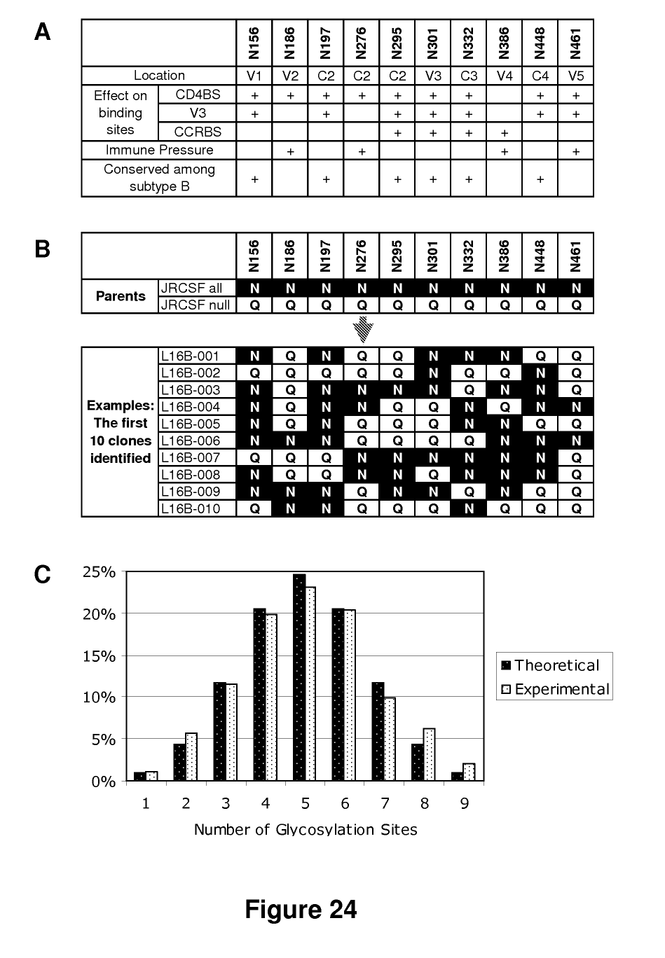

Reitter et al. (supra) showed that an SIV virus lacking three N-linked glycosylation sites induced more potent neutralizing antibodies. However, comprehensive and systematic studies of the immunogenicity of glycosylation mutants of HIV-1 Env have not been carried out. FIG. 24A summarizes the results of various studies which were performed previously on primary isolate and laboratory-adapted HIV-1 virus strains that have identified up to 10 glycosylation sites within the gp120 moiety of Env (N156, N188, N197, N276, N295, N301, N332, N386, N448, and N461) that can affect the sensitivity of the virus to neutralization by monoclonal or polyclonal antibodies (Koch, M. et al., supra; Ly, A., and L. Stamatatos, supra; McCaffrey, R. A. et al., supra; Reynard, F. et al., supra).

### A. Use of DNA Shuffling to Create a Library of JRCSF gp140 Glycosylation Variants

The objective of present work was to systematically test whether certain combination of glycosylation mutations at these ten sites can generate Env immunogens that are more effective than the wild-type sequence in inducing neutralizing antibodies against the homologous viruses. We synthesized the JRCSF env gene (using a human codon set) encoding fully glycosylated JRCSF gp140 protein (called JRCSFₐₗₗ; SEQ ID NO:111). A second sequence was synthesized that encodes JRCSF gp140 with ten N→Q mutations in the glycosylation motif (N-X-S/T) to eliminate N-linked glycosylation at 10 sites (JRCSFₙᵤₗₗ; SEQ ID NO:112) (FIG. 24B). The JRCSFₐₗₗ and JRCSFₙᵤₗₗ genes were shuffled and the resulting library was screened using a tiered DNA sequencing strategy to identify novel combinations of N-glycosylation patterns at the 10 sites while ensuring a 100% correct backbone sequence elsewhere. A total of 191 variants with unique glycosylation patterns were identified.

Visual inspection of ten exemplary gp140 glycosylation variants (shown in FIG. 24B, designated L16B-001 through L16B-010 and having the sequences SEQ ID NOs:113-122, respectively) suggested that recombination could (i) occur between any two adjacent glycosylation sites and (ii) occur multiple times with any given variant. The distribution of the number of glycosylation sites on these library variants agrees with the theoretical expectation (FIG. 24C). If all possible recombination events between JRCSFₐₗₗ and JRCSFₙᵤₗₗ genes occur, a total of 2¹⁰ or 1,024 combinatory possibilities will be formed. Since we isolated only a subset (191) of the total possible number of variants, we sought to determine if they were representative of the expected theoretical distribution.

We thus compared the distribution of the 191 experimental variants to that of the 1,024 theoretical combinatory possibilities. Besides JRCSFₐₗₗ and JRCSFₙᵤₗₗ which have all ten or zero N-glycans at the ten designated glycosylation site, the remaining combinatory possibilities can be divided into nine groups that have 1 to 9 out of the 10 N-glycans remaining intact. For each of the nine groups, the number of theoretical combinatory possibilities was calculated based on binomial coefficient. The theoretical distribution of the number of glycosylation sites was expressed as the percent over the total possibilities as shown in FIG. 24C. The distribution of our 191 variants tracked closely with the theoretical distribution, suggesting that recombination between JRCSFₐₗₗ and JRCSFₙᵤₗₗ occurred randomly and that the 191 variants isolated, as well as the entire library, were representative of the total theoretical possibilities.

### B. Immunization Screening to Identify Glycosylation Variants with Significantly Enhanced Ability to Neutralize JRCSF

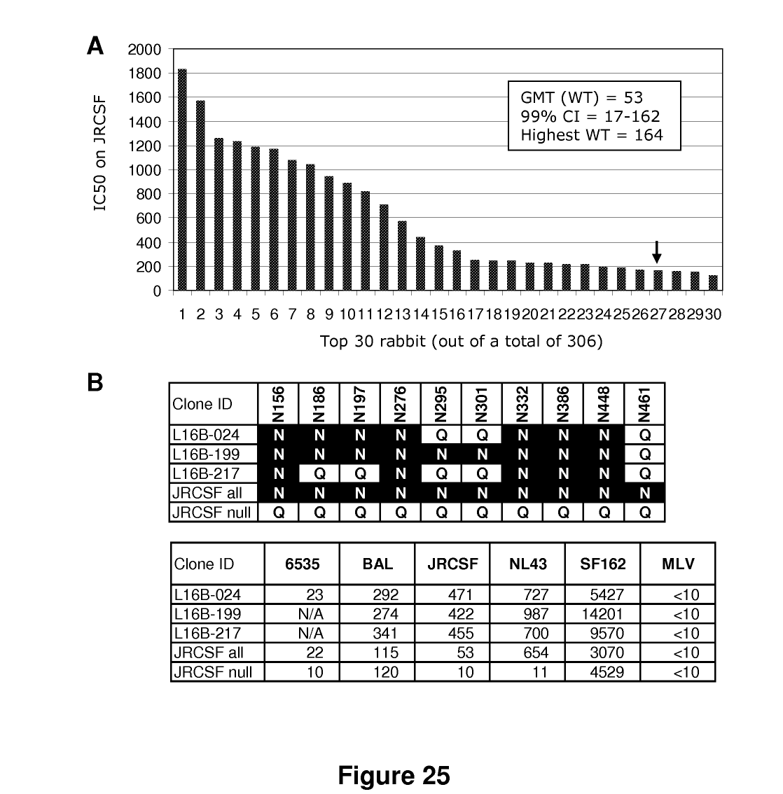

Immunization screening identified three variants with a significantly enhanced ability to neutralize JRCSF. Although different in glycosylation pattern, all three shared an N-glycan modification (N461Q) located within the V5 domain (FIGS. 25A and 25B).

Out of the 191 experimental variants, we selected 99 variants representative of each of the nine groups based on their expression-level and CD4-binding activity and immunized 3 rabbits per variant, along with both JRCSFₐₗₗ and JRCSFₙᵤₗₗ as controls. Analysis of the resultant day 98 sera showed that more that 20 individual rabbits showed a stronger autologous neutralization response to JRCSF than the best rabbit immunized with JRCSFₐₗₗ (FIG. 25A). The best autologous neutralization response achieved by the glycosylation variants is ˜10-fold higher than the best response achieved by the parental immunogen (FIG. 25A).

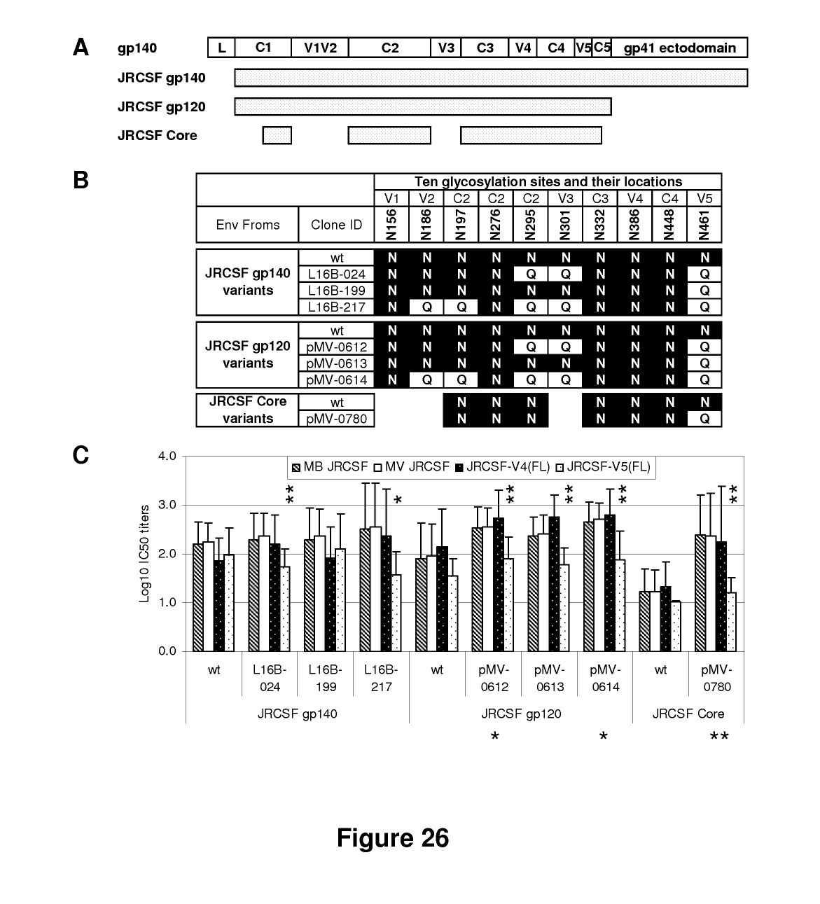

We identified three gp140 variants, L16B-024 (SEQ ID NO:123), L16B-199 (SEQ ID NO:124) and L16B-217 (SEQ ID NO:125) that were capable of eliciting autologous neutralizing responses that were statistically higher than that achieved by the parental immunogen JRCSFall (FIGS. 25B & C). A comparison of their glycosylation patterns reveals that although two of them have N→Q mutations in the V2, C2, and/or V3 regions, all three of them share a N461Q mutation located in the V5 region (FIG. 25B and FIG. 26B). This observation reinforces our previous observation that the V5 domain is a major JRCSF autologous neutralization epitope (Example 9) and implies that removal of the N-glycan alone might be sufficient to expose the JRCSF autologous neutralization epitope.

### C. Deglycosylation of the V5 Domain Significantly Enhances the Immunogenicity of the JRCSF Autologous Neutralization Epitope

Although some gp140 glycovariant immunogens showed an improvement in the autologous neutralization response compared to the wild-type gp140 immunogens, we have also observed that autologous activity can be elicited by different forms of Env (gp140, gp120, and Core, depicted in FIG. 26A). We therefore wanted to determine how deglycosylation, particularly at the N461 site of the V5 domain, within the gp140, gp120, or Core immunogens might affect the autologous response. Therefore, we performed immunizations with ten different constructs, including wild-type and glycovariant Env immunogens in the gp140, gp120, and Core formats, with ten groups of rabbits, each group containing eight rabbits. The immunogens used were the JRCSF gp140 variants L16B-024 (SEQ ID NO:123), L16B-199 (SEQ ID NO:124) and L16B-217 (SEQ ID NO:125); the JRCSF gp120 variants pMV-0612 (SEQ ID NO:126), pMV-0613 (SEQ ID NO:127), and pMV-0614 (SEQ ID NO:128), and the JRCSF core variant pMV-0780 (SEQ ID NO:129), as shown in FIG. 26B. This parallel comparison of glycovariant constructs in different Env forms allowed us to evaluate how different deglycosylated immunogens induce autologous neutralizing activity.

We found that deglycosylated gp120 and Core Env forms induced greater autologous JRCSF activity over the wild-type gp120 and Core immunogens, respectively, compared to the difference in autologous activity between deglycosylated and wild-type gp140 immunogens (FIG. 26C). Immunization of the gp120 glycosylation variants pMV-0612 and pMV-0614 (see FIG. 26B) induced a statistically significant increase in neutralization of JRCSF compared to the wild-type gp120 construct. Furthermore, neutralization of chimeric pseudoviruses shows the autologous activity elicited by gp120 glycosylation variants is directed against the V5 domain, as there is a reduction in neutralization of the JRCSF V5 (FL) pseudovirus by these antisera.

Immunization with the Core variant containing the N461Q mutation (plasmid pMV-0780) resulted in a large improvement in JRCSF neutralization compared to immunization with wild-type Core. This improvement appears to be due to autologous activity directed against the V5 epitope as demonstrated by the loss of neutralization activity on JRCSF V5 (FL). We did not observe a statistically significant improvement in neutralization of the JRCSF pseudovirus by sera from rabbits immunized with gp140 glycosylation variants compared to wild-type gp140ₐₗₗ. However, for gp140 glycosylation variants L16B-024 and L16B-217, it is clear that autologous activity is directed against V5, as neutralization of JRCSF V5 (FL) is reduced compared to the JRCSF pseudovirus for these sera.

Overall, these results demonstrate that deglycosylation enhances the autologous neutralization activity of the gp120 and Core forms of Env immunogens and that most of the autologous response elicited by these immunogens is directed against the V5 domain. Our results also suggested that, among the three Env forms, gp140, gp120 and gp120 Core, gp120 Core is the best platform to study how glycosylation might influence the immunogenicity of autologous neutralizing epitope(s) located on the silent face of gp120 (FIG. 26C).

### D. Deglycosylation of the V5 Epitope Renders a JRCSF Pseudovirus More Sensitive to Neutralization

A JRCSF pseudovirus with the N461Q mutation in Env was created. This mutation did not appear to be deleterious for proper Env production or stability since the pseudovirus was produced to the same level as that containing the wild-type JRCSF Env sequence (data not shown). Previous studies by others have shown that the extensive glycosylation of Env on the surface of viral particles can interfere with neutralization, potentially shielding antigenic sites from antibody binding (Derdeyn, C. A. et al., supra; Koch, M. et al., supra; Ly, A., and L. Stamatatos, supra; McCaffrey, R. A. et al., supra; Reynard, F et al., supra; Song, B. et al., supra; and Wei, X. et al., supra. Since V5 contains a glycosylation site at position Asn 461, removal of which increases the immunogenicity of the autologous epitope(s) that are largely located in the V5 domain (see above), we sought to gain insight into how glycosylation at N461 affected sensitivity to the autologous neutralization activity directed against the V5 domain.

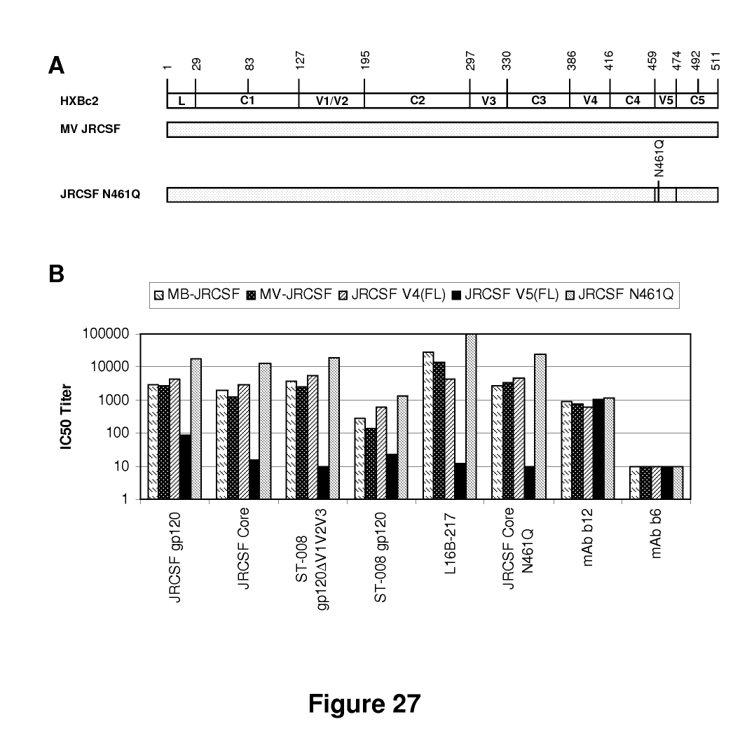

We generated a JRCSF Env construct with the N461Q mutation in V5 to remove N-linked glycosylation at this position (FIG. 27A), and this construct was used to generate the pseudovirus, JRCSF N461Q, which we tested for neutralization by sera which are known to have autologous neutralization activity directed against the V5 domain.

FIG. 27B shows that a JRCSF pseudovirus lacking the glycosylation site at N461 is 7-10 fold more sensitive, compared to the wt JRCSF pseudoviruses MB-JRCSF and MV-JRCSF, to neutralization by antisera which was prepared by immunization with various JRCSF-based immunogens. This result, to the best of our knowledge, is the first direct demonstration that deglycosylation of a neutralization epitope can increase the susceptibility of the virus to autologous neutralization activity. Our results furthermore suggest that the presence of the N461 N-linked glycan could be the result of a viral evasion mechanism occurring during natural infection (Liu, S. et al., AIDS Res. Hum. Retroviruses 24:521-527 (2008); and Wei, X. et al., Nature 422:307-12 (2003)).

To summarize, our results demonstrate that removal of the N641 N-glycan (i) increases the neutralization susceptibility of JRCSF pseudovirus (a measure of antigenicity) and (ii) enhances the autologous neutralization response towards the pseudovirus (a measure of immunogenicity). This represents the first direct evidence showing that the antigenicity and the immunogenicity of an HIV-1 Env can be directly correlated.

### Example 12

The following example demonstrates that DNA shuffling can create gp120 Core variants which expose V1V2V3-independent autologous neutralization epitope(s) that are otherwise cryptic on the wild-type gp120 Core immunogen.

### A. Not All gp120-Based Autologous Neutralization Epitopes Located on the Silent Face are Immunogenic in the Wild-Type Sequence

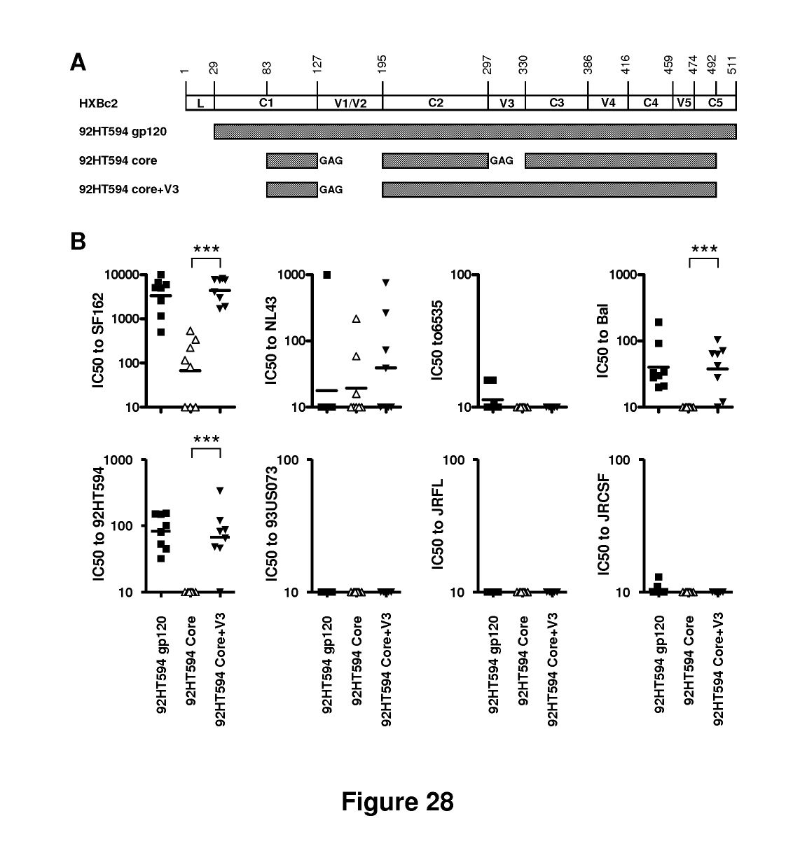

Autologous neutralizing antibodies are not simply generated from autologous Core immunogens. For example, FIGS. 28A-B shows an experiment, similar to that of FIGS. 17A-B, using three immunogens (gp120, Core, and Core+V3) based on the 92HT594 strain of HIV-1. The 92HT594 gp120 immunogen induces weak autologous neutralization activity against the 92HT594 pseudovirus which appears to be based on the V3 epitope, based on the comparable neutralization activity of 92HT594 Core+V3 immunogen antiserum against the 92HT594 pseudovirus. However, the 92HT594 Core immunogen (which lacks the V3 domain) fails to induce autologous neutralization activity against the 92HT594 pseudovirus (FIG. 28B).

### B. DNA Shuffling of the gp120 Core Can Expose Previously Cryptic Autologous Neutralization Epitope(s)

The inability of the 92HT594 Core immunogen to induce an autologous neutralization response raised the question of whether DNA shuffling could create variants containing 92HT594 sequences that would expose epitopes which are required for autologous neutralization of that strain. To investigate this idea, we shuffled Core sequences from eight known strains of HIV-1, including the 92HT594 and JRCSF strains, and obtained gp120 Core variant polypeptides as described in Example 4. Plasmids expressing these Core variant sequences were used to immunize rabbits as described above, using the homologous Core variant proteins for boosting at day 84.

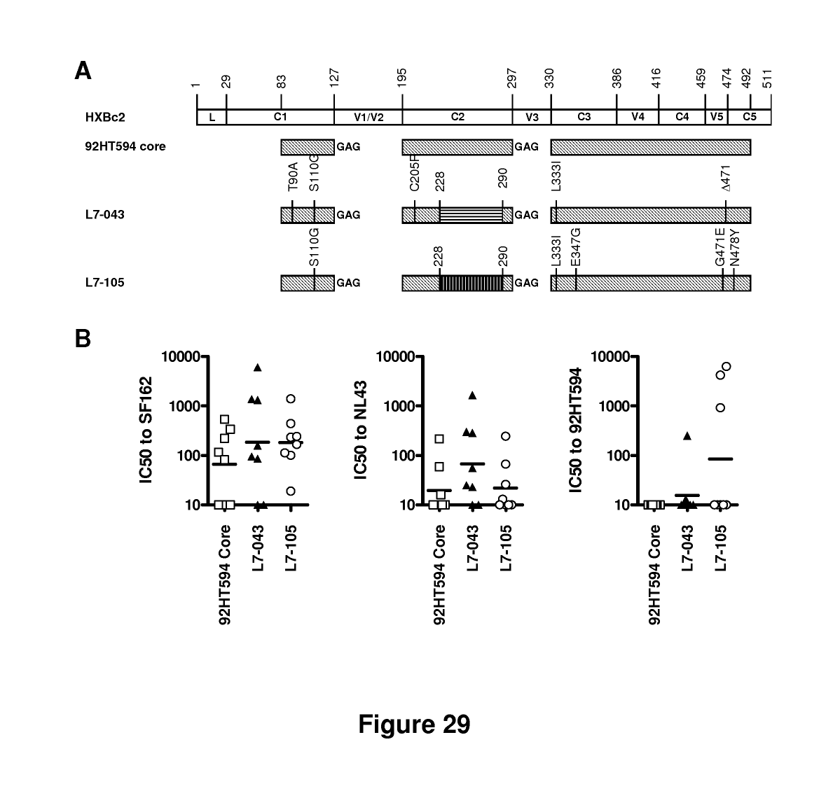

Two of these Core variants, L7-043 (SEQ ID NO:10) and L7-105 (SEQ ID NO:14), induced autologous neutralizing antibodies against the 92HT594 virus, while the 92HT594 Core immunogen did not (FIG. 29B). In contrast, all three Core immunogens induced antibodies with similar neutralizing activities against the SF162 and NL43 viruses (FIG. 29B), suggesting such change is not global but rather epitope-specific. Both the L7-043 and L7-105 variants thus contain non-V3 based autologous neutralization epitope(s) that is/are cryptic in the wild-type 92HT594 Core immunogen. L7-105 appears to give a stronger neutralizing response to 92HT594 than does L7-043 (FIG. 29B), probably due to subtle difference since they differ by only 9 amino acids (3% overall). The strong neutralizing responses by some rabbits do not result in statistically significant differences for the L7-043 and L7-105 groups as a whole. However, the variable nature of the immune response to the autologous neutralization epitope of 92HT594 was similar to that of JRCSF autologous epitopes based on V5 and V4 (see, e.g., the autologous neutralization responses against the JRCSF pseudovirus obtained by the JRCSF gp120 Core immunogen in FIG. 17B).

### C. The 92HT594 Autologous Neutralization Epitope Involves the V4 Domain

Sequence comparisons show that, except for a region from amino acids 228 through 290 containing sequences not derived from 92HT594, both the L7-043 and L7-105 variants contain mostly 92HT594 Env sequence with limited point mutations as indicated in FIG. 29A. The V4 and V5 domains are identical to that of 92HT594 (FIG. 29A), suggesting the potential involvement of these regions in the autologous neutralization activity against 92HT594. To investigate whether the V4 and V5 domains of 92HT594 are responsible for the autologous neutralization activity against the 92HT594 virus, we created three variants using L7-043 immunogen as a backbone, in which the V4 and/or V5 sequences were replaced with those of JRCSF (FIG. 30A). Each of the three constructs, L7-043 V4V5 JR (SEQ ID NO: 130), L7-043_V4 JR (SEQ ID NO:131) and L7-043_V5 JR (SEQ ID NO:132) along with controls immunogens JRCSF gp120 Core, 92HT594 gp120 Core, and L7-043, were used to immunize 8 rabbits each.

Replacement of the 92HT594 V5 domain with the JRCSF V5 domain in the L7-043_V5 JR immunogen did not diminish the immunogenicity of the 92HT594 autologous neutralization epitope, suggesting that the V5 domain does not play a major role in the autologous neutralization of the 92HT594 virus (FIG. 30B). Replacing the 92HT594 V4 domain with the JRCSF V4 domain in the L7-043_V4 JR immunogen did, however, abolish its neutralization activity against the 92HT594 virus, suggesting that V4 is involved in the formation of the 92HT594 autologous neutralizing epitope (FIG. 29B). The fact that the V4 swap did not affect the neutralization responses of the L7-043_V4 JR immunogen against the heterologous viruses SF162 and NL43 (compared to the neutralization responses induced by the JRCSF Core, 92HT594 Core, and L7-043 immunogens against these viruses) suggests that the V4 replacement did not have a significant global effect on immunogenicity (FIG. 30B). Therefore, the involvement of the V4 domain in the autologous neutralization of the 92HT594 virus is more likely due to specific sequences within or around V4 loop.

Interestingly, replacing 92HT594 V5 with JRCSF V5 in the L7-043_V5 JR immunogen was sufficient to generate an autologous neutralization response against the JRCSF virus, which was as strong as that induced by the JRCSF Core immunogen (FIG. 30B), suggesting that the V5 domain of JRCSF alone is a highly immunogenic autologous neutralizing epitope. In contrast, the fact that the V4 swap in L7-043_V4 JR did not produce any detectable neutralization response against the JRCSF virus (FIG. 30B) suggests that V4 alone is not sufficient for the V4-based autologous neutralization activity against JRCSF. These results confirm and extend our conclusions on the nature of the JRCSF autologous neutralization epitope discussed in Example 9.

### Example 13

The following example demonstrates that multiple structural modifications may enhance the autologous neutralization potency of immunogen polypeptides.

Sequence comparison of the two variant Core polypeptides, L7-043 and L7-105, revealed they differ in only nine amino acid positions, none of which are in the V4 domain. Nonetheless, the best autologous neutralization response against the 92HT594 virus induced by the L7-105 immunogen is 10-fold better than that induced by the L7-043 immunogen (FIG. 29B), even though, as demonstrated in Example 13 above, both of these polypeptides induce autologous immunization through the V4 domain. This suggests that sequence differences outside of the V4 domain play a role in enhancing the immunogenicity of the 92HT594 autologous neutralizing epitope in the variant polypeptide immunogens.

The L7-043_V5 JR immunogen, in which 92HT594 V5 was replaced with JRCSF V5, induced a neutralizing response against the 92HT594 virus that was 50-fold stronger than that induced by the L7-043 immunogen (p<0.0001, FIG. 30B), suggesting that inclusion of the heterologous JRCSF V5 domain sequence results in a better presentation of the 92HT594 autologous epitope to the immune system. The potency and homogeneity of the neutralization response induced by the L7-043_V5 JR immunogen was also improved when tested against both the SF162 virus and the NL43 virus, compared to the neutralization response induced by the JRCSF Core, 92HT594 Core, and L7-043 immunogens against those same viruses (FIG. 30B). A similar phenomenon was also observed for L7-043_V4V5 JR, suggesting that replacing either the V5 or the V4V5 domains may have a positive global effect on epitopes located outside the V4 and V5 domains.

L7-043_V5 JR also induced an autologous response against the JRCSF virus (FIG. 30B). In some rabbits immunized with the L7-043_V5 JR immunogen (which as noted above contains the 92HT594 V4 domain and the JRCSF V5 domain), a strong dual neutralization response against both the JRCSF virus and the 92HT594 virus was induced (FIG. 30B). The results demonstrate for the first time that two specific autologous neutralization epitopes are simultaneously immunogenic when combined into a single immunogen backbone (e.g., the L7-043_V5-JR Core construct).

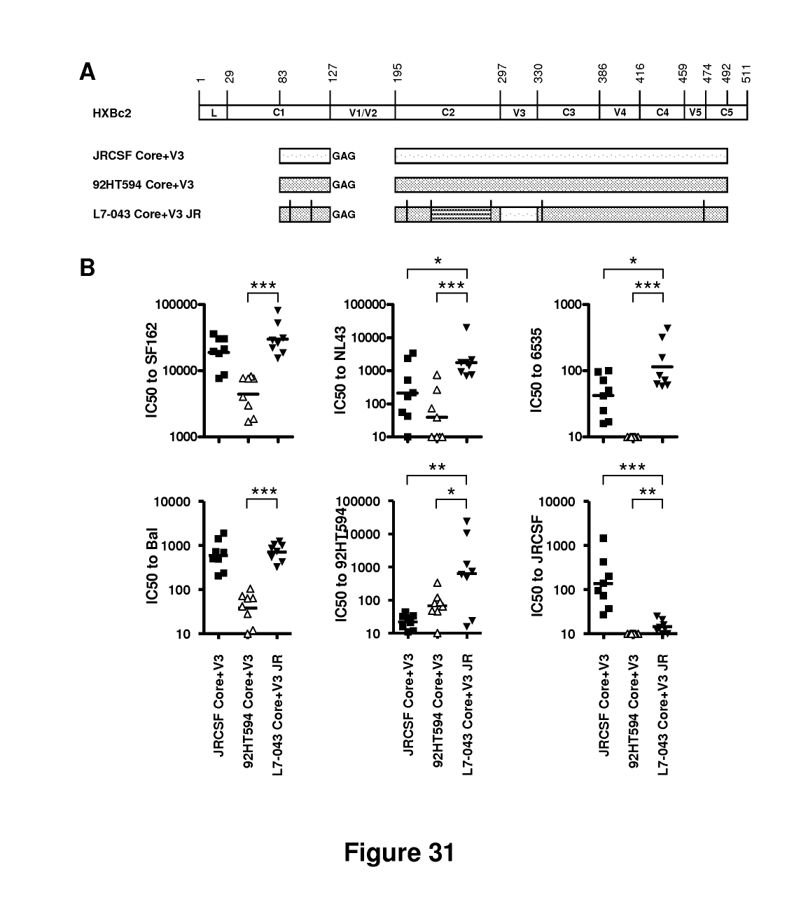

In a separate experiment, we tested if the anti-V3 neutralization response could be combined with the autologous neutralization response against the 92HT594 virus. Since the V3 loop of 92HT594 was not as immunogenic as that of JRCSF (compare FIGS. 17A-B to FIGS. 28A-B), we inserted the V3 domain sequence of JRCSF into L7-043 to generate L7-043 Core+V3 JR (SEQ ID NO:134, FIG. 31A). We used the L7-043 Core+V3 JR immunogen to immunize eight rabbits and analyzed the day 98 sera for their neutralizing activities against a panel of 6 pseudoviruses. Our results suggest that the immune responses against JRCSF domain V3 and 92HT594 domain V4 not only can be combined on a single Core+V3 construct, but also can have a synergistic effect on each other.

Compared to the JRCSF Core+V3 immunogen, the L7-043 Core+V3 JR immunogen induced either equally strong or a significantly better V3-based neutralization response (p<0.05 for 6535; p<0.05 for NL-43; FIG. 31B), suggesting that immunogenicity of the JRCSF V3 domain epitope(s) was enhanced in the L7-043 Core+V3 JR immunogen construct.

The L7-043 Core+V3 JR immunogen also induced a significantly better neutralizing response against the 92HT594 virus than did JRCSF Core+V3 immunogen (p<0.0001; FIG. 31B), suggesting that the JRCSF V3-based neutralization response against the 92HT594 virus does not play a major role in the autologous neutralization of the 92HT594 virus. However, when compared to the L7-043 immunogen, the neutralization response against the 92HT594 virus induced by the L7-043 Core+V3 JR immunogen is 50-fold stronger (p=0.002), suggesting that the insertion of the JRCSF V3 sequence into the L₇-043 backbone enhances the immunogenicity of the autologous neutralization epitope of 92HT594, similar to what was observed with the JRCSF V5 domain replacement in the L7-043_V5 JR construct (FIG. 30B).

In summary, our results show that modifications to the shuffled Core variants can have synergistic effects on the immunogenicity of various neutralizing epitopes. Additionally, the combination of various neutralization antibodies may also act cooperatively or synergistically in the neutralization of a given virus or viruses. Similar synergy in neutralization was previously observed between anti-V3 and anti-CD4 antibodies and among anti-V2, anti-V3 and anti-CD4 antibodies (Montefiori, D. et al., PLoS Medicine 4(12):1867-1871 (2007); Thali, M. et al., J. Acquir. Immun. Defic. Syndr. 5:591-599 (1992); Tilley, S. A. et al., AIDS Res. Hum. Retroviruses 8:461-467 (1992); and Vijh-Warrier, S. et al., J. Virol. 70:4466-4473 (1996)). The constructs resulting from these modification approaches can be used as components or platforms to develop polyvalent vaccines against HIV-1.

While the foregoing invention has been described in some detail for purposes of clarity and understanding, it will be clear to one skilled in the art from a reading of this disclosure that various changes in form and detail can be made without departing from the true scope of the invention. It is understood that the materials, examples, and embodiments described herein are for illustrative purposes only and not intended to be limiting and that various modifications or changes in light thereof will be suggested to persons skilled in the art and are to be included within the spirit and purview of this application and scope of the appended claims. All publications, patent applications, patents, or other documents mentioned herein are incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including definitions, will control.

What is claimed is:

1. An isolated or recombinant polypeptide comprising a polypeptide sequence having at least 95% sequence identity to a sequence selected from the group consisting of SEQ ID NOS:1-21 and 56-63, wherein the polypeptide
(a) induces an immune response against at least one human immunodeficiency virus type 1 (HIV-1) or HIV-1 pseudovirus in a subject to whom an effective amount of the polypeptide is administered; and/or
(b) binds to an HIV-1 neutralizing antibody.

2. The polypeptide of claim 1, wherein the polypeptide induces an immune response against at least two HIV-1 viruses or HIV-1 pseudoviruses.

3. The polypeptide of claim 2, wherein the at least two HIV-1 viruses or HIV-1 pseudoviruses comprise the same HIV-1 virus subtype.

4. The polypeptide of claim 2, wherein the at least two HIV-1 viruses or HIV-1 pseudoviruses comprise different HIV-1 virus subtypes.

5. The polypeptide of claim 1, wherein the polypeptide induces an immune response that prevents infection of cells by at least one HIV-1 virus.

6. The polypeptide of claim 1, wherein the immune response comprises production of antibodies against at least one HIV-1 virus or HIV-1 pseudovirus.

7. The polypeptide of claim 6, wherein the immune response comprises production of neutralizing antibodies against at least one HIV-1 virus or HIV-1 pseudovirus.

8. The polypeptide of claim 7, wherein the polypeptide induces production of neutralizing antibodies against at least two HIV-1 viruses or at least two HIV-1 pseudoviruses in the subject.

9. The polypeptide of claim 8, wherein each of the at least two HIV-1 viruses comprises the same HIV-1 virus subtype.

10. The polypeptide of claim 9, wherein each of the at least two HIV-1 viruses comprises an HIV-1 subtype B virus.

11. The polypeptide of claim 10, wherein the at least two HIV-1 subtype B viruses are selected from the group consisting of BAL, Bx08, QZ4589, 1196, JRCSF, 92HT594, 692, 93US073, NL43, JRFL, and SF-162.

12. The polypeptide of claim 8, wherein each of the at least two HIV-1 pseudoviruses comprises a gp160 envelope protein of an HIV-1 virus.

13. The polypeptide of claim 12, wherein each of the at least two HIV-1 pseudoviruses comprises a gp160 envelope protein of an HIV-1 virus of the same subtype.

14. The polypeptide of claim 13, wherein each of the at least two HIV-1 pseudoviruses comprises a gp160 envelope protein of an HIV-1 subtype B virus.

15. The polypeptide of claim 14, wherein each of the at least two HIV-1 pseudoviruses comprises a gp160 envelope protein of an HIV-1 subtype B virus selected from the group consisting of BAL, Bx08, QZ4589, 1196, JRCSF, 92HT594, 692, 93US073, NL43, JRFL, and SF-162.

16. The polypeptide of claim 7, wherein the polypeptide induces the production of neutralizing antibodies in a subject to whom an effective amount of the polypeptide is administered, wherein the neutralizing antibodies are active against at least two HIV-1 pseudoviruses, each pseudovirus comprising a gp160 envelope polypeptide of a different HIV-1 virus.

17. The polypeptide of claim 1, wherein the polypeptide induces in a subject to whom it is administered an immune response that is cross reactive against at least two different HIV-1 viruses.

18. The polypeptide of claim 7, wherein the polypeptide induces in a subject to whom an effective amount of the polypeptide is administered the production of a titer of HIV-1 neutralizing antibodies that is greater than the titer of HIV-1 neutralizing antibodies induced in the subject by administration of an equal amount of a recombinant WT HIV-1 gp120 polypeptide.

19. The polypeptide of claim 1, wherein the polypeptide has a binding affinity for the HIV-1 neutralizing antibody that is greater than the binding affinity of a recombinant WT HIV-1 gp120 polypeptide for the HIV-1 neutralizing antibody.

20. The polypeptide of claim 1, wherein the HIV-1 neutralizing antibody is monoclonal antibody b12 or 2G12.

21. The polypeptide of claim 19, wherein the polypeptide has a binding affinity for an HIV-1 non-neutralizing antibody that is lower than the binding affinity of a recombinant WT HIV-1 gp120 polypeptide for the HIV-1 non-neutralizing antibody.

22. The polypeptide of any of claims 18-21, wherein the recombinant WT HIV-1 gp120 polypeptide is the recombinant HIV-1 gp120 polypeptide of HIV-1_{JRCSF} (SEQ ID NO:80).

23. The polypeptide of claim 21, wherein the polypeptide has (1) a binding affinity for the HIV-1 non-neutralizing antibody that is lower than the binding affinity of the recombinant gp120 envelope polypeptide of HIV-1_{JRCSF} for the HIV-1 non-neutralizing antibody and (2) a binding affinity for the HIV-1 neutralizing antibody that is greater than the binding affinity of the recombinant gp120 envelope polypeptide of HIV-1_{JRCSF} for the HIV-1 neutralizing antibody.

24. The polypeptide of any of claims 21-23, wherein the HIV-1 non-neutralizing antibody is monoclonal antibody b3 or b6.

25. The polypeptide of claim 21, wherein the polypeptide exhibits a b12/b6 binding affinity ratio that is greater than the b12/b6 binding affinity ratio of a recombinant HIV-1 gp120 polypeptide, wherein the recombinant WT HIV-1 gp120 polypeptide is the recombinant HIV-1 gp120 polypeptide of HIV-1_{JRCSF}.

26. The polypeptide of claim 1, wherein the polypeptide comprises a sequence having at least 98% sequence identity to a sequence selected from the group consisting of SEQ ID NOS:1-21 and 56-63.

27. The polypeptide of claim 33, wherein the polypeptide comprises a sequence selected from the group consisting of SEQ ID NOS:1-21 and 56-63.

28. An isolated or recombinant polypeptide comprising a fragment of a gp120 variant polypeptide sequence, said gp120 variant polypeptide sequence having at least 95% sequence identity to a sequence selected from the group consisting of SEQ ID NOS:1-7 and 56-63, wherein the fragment comprises at least those amino acid residues of the gp120 variant polypeptide sequence located at positions corresponding by reference to amino acid residues of regions C2, C3, V4, C4, and V5 of the recombinant HIV-1 gp120-HXB2 envelope protein sequence (SEQ ID NO:54) shown in FIGS. 10A-10F, wherein the amino acid residues of the fragment are numbered by reference to amino acid residues of the recombinant gp120-HXB2 envelope protein, and
wherein the polypeptide induces an immune response against at least one HIV-1 virus or pseudovirus in a subject to whom an effective amount of the polypeptide is administered.

29. The polypeptide of claim 28 wherein the gp120 variant polypeptide sequence has at least 98% sequence identity to a sequence selected from the group consisting of SEQ ID NOS:1-7 and 56-63.

30. The polypeptide of claim 29 wherein the gp120 variant polypeptide sequence is selected from the group consisting of SEQ ID NOS:1-7 and 56-63.

31. The polypeptide of any of claims 28-30, wherein the fragment further comprises amino acid residues of the gp120 variant polypeptide sequence which correspond to amino acid residues 83-127 of the C1 region of the recombinant HXB2 gp120 envelope protein (SEQ ID NO:54) shown in FIGS. 10A-10F.

32. The polypeptide of any of claims 28-31, wherein the fragment further comprises amino acid residues of the gp120 variant polypeptide sequence which correspond to amino acid residues 472-492 of the C5 region of the recombinant HXB2 gp120 envelope protein (SEQ ID NO:54) shown in FIGS. 10A-10F.

33. The polypeptide of claim 31, wherein the fragment further comprises amino acid residues of the gp120 variant polypeptide sequence which correspond to amino acid residues 29-82 of the C1 region of the recombinant HXB2 gp120 envelope protein (SEQ ID NO:54) shown in FIGS. 10A-10F.

34. The polypeptide of claim 32, wherein the fragment further comprises amino acid residues of the gp120 variant polypeptide sequence which correspond to amino acid residues 493-511 of C5 region of the recombinant HXB2 gp120 envelope protein (SEQ ID NO:54) shown in FIGS. 10A-10F.

35. The polypeptide of claim 28, wherein the recombinant gp120-HXB2 envelope protein (SEQ ID NO:54) comprises the following regions:
(a) the C2 region comprises amino acid residues 197 to 295;
(b) the C3 region comprises amino acid residues 332 to 384;
(c) the V4 region comprises amino acid residues 385 to 418;
(d) the C4 region comprises amino acid residues 419 to 459 or 419 to 460; and
(e) the V5 region comprises amino acid residues 460 to 471 or 461 to 471.

36. The polypeptide of claim 28, wherein the regions of the polypeptide are covalently linked in the same order as domains C2, C3, V4, C4, and V5 of the recombinant HIV-1 gp120-HXB2 envelope protein shown in FIGS. 10A-10F.

37. The polypeptide of any of claims 28-36, wherein the immune response comprises production of neutralizing antibodies against at least one HIV-1 virus in the subject.

38. The polypeptide of any of claims 28-37, wherein the polypeptide induces neutralizing antibodies against at least two HIV-1 viruses of the same subtype.

39. The polypeptide of any of claims 28-37, wherein the polypeptide induces neutralizing antibodies against at least two HIV-1 viruses of different subtypes.

40. The polypeptide of claim 39, wherein the HIV-1 viruses of different subtypes are selected from the group consisting of subtype A, subtype B, subtype C, subtype E, subtype F, and subtype G.

41. The polypeptide of any of claims 28-40, wherein the polypeptide induces the production of neutralizing antibodies in a subject to whom an effective amount of the polypeptide is administered, wherein the neutralizing antibodies are active against at least two HIV-1 pseudoviruses, each pseudovirus comprising a gp160 envelope polypeptide of a different HIV-1 virus.

42. The polypeptide of claim 41, wherein each gp160 envelope polypeptide is of a different HIV-1 virus subtype.

43. The polypeptide of any of claims 28-42, wherein the polypeptide induces in a subject to whom an effective amount of the polypeptide is administered the production of a titer of HIV-1 neutralizing antibodies that is greater than the titer of HIV-1 neutralizing antibodies induced in the subject by administration of an equal amount of a recombinant WT HIV-1 gp120 polypeptide.

44. The polypeptide of claim 43, wherein the recombinant WT HIV-1 gp120 polypeptide is the recombinant HIV-1 gp120 polypeptide of HIV-1_{JRCSF} (SEQ ID NO:80).

45. An isolated or recombinant polypeptide comprising a first, a second, a third, a fourth and a fifth subsequence of a gp120 variant sequence, the gp120 variant sequence comprising a an amino acid sequence having at least 95% sequence identity to a polypeptide sequence selected from the group consisting of SEQ ID NOS:1-21 and 56-63, wherein:
(a) the first subsequence of the gp120 variant sequence comprises a sequence corresponding by reference to amino acid residues 83-127 of the C1 region of the recombinant HXB2 gp120 envelope protein (SEQ ID NO:54) shown in FIGS. 10A-10F, wherein the C-terminus of the first subsequence is covalently linked by a peptide bond to the N-terminus of a first linker peptide;
(b) the second subsequence of the gp120 variant sequence corresponds by reference to the C2 region of the recombinant HXB2 gp120 envelope protein (SEQ ID NO:54) shown in FIGS. 10A-10F, wherein the N-terminus of the second subsequence is covalently linked by a peptide bond to the C-terminus of the first linker peptide, and the C-terminus of the second subsequence is covalently linked by a peptide bond to the N-terminus of a second linker peptide or a gp120 V3 region sequence;
(c) the third subsequence of the gp120 variant sequence corresponds by reference to the C3 region of the recombinant HXB2 gp120 envelope protein (SEQ ID NO:54) shown in FIGS. 10A-10F, and the N-terminus of the third subsequence is covalently linked by a peptide bond to the C-terminus of the second linker polypeptide or the gp120 V3 region sequence, and the C-terminus of the third subsequence is covalently linked by a peptide bond to the N-terminus of a gp120 V4 region sequence;
(d) the fourth subsequence of the gp120 variant sequence corresponds by reference to the C4 region of the recombinant HXB2 gp120 envelope protein (SEQ ID NO:54) shown in FIGS. 10A-10F, and the N-terminus of the fourth subsequence is covalently linked by a peptide bond to the C-terminus of the gp120 V4 region sequence, and the C-terminus of the fourth subsequence is covalently linked by a peptide bond to the N-terminus of a gp120 V5 region sequence; and
(e) the fifth subsequence of the gp120 variant sequence comprises a sequence corresponding by reference to amino acid residues 472-492 of the C5 region of the recombinant HXB2 gp120 envelope protein (SEQ ID NO:54) shown in FIGS. 10A-10F, and the N-terminus of the fifth subsequence is covalently linked by a peptide bond to the C-terminus of the V5 region sequence;
wherein the gp120 V3 region sequence, the gp120 V4 region sequence and the gp120 V5 region sequence correspond by reference to the V3 region, the V4 region, and the V5 region, respectively, of the recombinant HXB2 gp120 envelope protein (SEQ ID NO:54) shown in FIGS. 10A-10F, and one or more of the gp120 V3 region sequence, the gp120 V4 region sequence and the gp120 V5 region sequence is not a subsequence of the selected gp120 variant sequence; and wherein the polypeptide induces an immune response against at least one HIV-1 virus or pseudovirus in a subject to whom an effective amount of the polypeptide is administered.

46. The polypeptide of claim 45, wherein the first and second linker peptides comprise the amino acid sequence Gly-Ala-Gly.

47. The polypeptide of any of claims 45-46, wherein one or more of the gp120 V3 region sequence, the gp120 V4 region sequence, and the gp120 V5 region sequence is a subsequence of (a) the amino acid sequence of a gp120 variant selected from the group consisting of SEQ ID NOS:1-21 and SEQ ID NOS:56-63 excluding the selected gp120 variant sequence or (b) the gp120 amino acid sequence of an HIV-1 strain, which subsequence corresponds by reference to the V3 region, the V4 region, or the V5 region, respectively, of the recombinant HXB2 gp120 envelope protein (SEQ ID NO:54) shown in FIGS. 10A-10F,
and wherein said one or more gp120 V3 region sequence, gp120 V4 region sequence, or gp120 V5 region sequence is not identical to the gp120 V3 region sequence, gp120 V4 region sequence, or gp120 V5 region sequence, respectively, of the selected gp120 variant sequence.

48. The polypeptide of claim 47, wherein the gp120 amino acid sequence of an HIV-1 strain is selected from the gp120 amino acid sequence of an HIV-1 subtype A strain, an HIV-1 subtype B strain, an HIV-1 subtype C strain, an HIV-1 subtype E strain, an HIV-1 subtype F strain, and an HIV-1 subtype G strain.

49. The polypeptide of claim 48, wherein the gp120 amino acid sequence is the gp120 amino acid sequence of an HIV-1 subtype B strain.

50. The polypeptide of claim 49, wherein the gp120 amino acid sequence of the HIV-1 subtype B strain is selected from the group consisting of the gp120 sequence of JRCSF (SEQ ID NO:80), 89.6 (SEQ ID NO:81), 92HT593 (SEQ ID NO:82), 92HT594 (SEQ ID NO:83), 92HT596 (SEQ ID NO:84), 92HT599 (SEQ ID NO:85), 92US657 (SEQ ID NO:86), 92US712 (SEQ ID NO:87), 92US727 (SEQ ID NO:88), and 93US073 (SEQ ID NO:89).

51. The polypeptide of claim 47, wherein two or three of the gp120 V3 region sequence, the gp120 V4 region sequence, and the gp120 V5 region sequence are subsequences of gp120 amino acid sequences of different HIV-1 viral strains.

52. The polypeptide of claim 51, wherein the two or three of the gp120 V3 domain sequence, the gp120 V4 domain sequence, and the gp120 V5 domain sequence are subsequences of gp120 sequences of different HIV-1 subtype B strains.

53. The polypeptide of any of claims 45-52, wherein the immune response comprises production of neutralizing antibodies against at least one HIV-1 virus in the subject.

54. The polypeptide of any of claims 45-52, wherein the polypeptide induces neutralizing antibodies against at least two HIV-1 viruses of the same subtype.

55. The polypeptide of any of claims 45-52, wherein the polypeptide induces neutralizing antibodies against at least two HIV-1 viruses of different HIV-1 virus subtypes.

56. The polypeptide of claim 55, wherein the different HIV-1 virus subtypes are selected from the group consisting of subtype A, subtype B, subtype C, subtype E, subtype F, and subtype G.

57. The polypeptide of any of claims 45-56, wherein the polypeptide induces the production of neutralizing antibodies in a subject to whom an effective amount of the polypeptide is administered, wherein the neutralizing antibodies are active against at least two HIV-1 pseudoviruses, each pseudovirus comprising a gp160 envelope polypeptide of a different HIV-1 virus.

58. The polypeptide of claim 57, wherein each gp160 envelope polypeptide is of a different HIV-1 virus subtype.

59. The polypeptide of any of claims 45-58, wherein the polypeptide induces in a subject to whom an effective amount of the polypeptide is administered the production of a titer of HIV-1 neutralizing antibodies that is greater than the titer of HIV-1 neutralizing antibodies induced in the subject by administration of an equal amount of a recombinant WT HIV-1 gp120 polypeptide.

60. The polypeptide of claim 59, wherein the recombinant WT HIV-1 gp120 polypeptide is the recombinant HIV-1 gp120 polypeptide of HIV-1_{JRCSF} (SEQ ID NO:80).

61. An isolated or recombinant polypeptide comprising a polypeptide sequence having at least 95% sequence identity to a sequence selected from the group consisting of SEQ ID NOS:1-21 and 56-63, wherein the polypeptide sequence comprises an amino acid substitution in a glycosylation motif (N-X-S/T) which eliminates N-linked glycosylation at one or more glycosylation sites selected from N156, N188, N197, N276, N295, N301, N332, N386, N448, and N461, wherein the amino acid residues are numbered according to the amino acid residues of the recombinant gp120-HXB2 envelope protein (SEQ ID NO:54) as shown in FIGS. 10A-10F, wherein the polypeptide
(a) induces an immune response against at least one human immunodeficiency virus type 1 (HIV-1) or HIV-1 pseudovirus in a subject to whom an effective amount of the polypeptide is administered; and/or
(b) binds to an HIV-1 neutralizing antibody.

62. The polypeptide of claim 61, wherein the amino acid substitution is a substitution of the N (Asn) in the glycosylation motif with a different amino acid.

63. The polypeptide of claim 62, wherein the amino acid substitution is a substitution of the N (Asn) in the glycosylation motif with a Q (Gln).

64. The polypeptide of claim 63, comprising the substitution N461Q.

65. The polypeptide of claim 61, wherein the amino acid substitution is a substitution of the Ser(S) or Thr(T) in the glycosylation motif with a different amino acid.

66. The polypeptide of claim 65, wherein the amino acid substitution is a substitution of the Ser(S) or Thr(T) in the glycosylation motif with an A (Ala).

67. The polypeptide of any of claims 59-64, comprising substitutions which eliminate N-linked glycosylation at two or more of said glycosylation sites.

68. The polypeptide of any of claims 61-67, wherein the polypeptide induces an increased immune response against at least one HIV-1 virus or pseudovirus compared to the immune response induced by the polypeptide lacking the substitution(s) which eliminate N-linked glycosylation at the one or more glycosylation sites.

69. A recombinant or isolated HIV-1 gp120 polypeptide variant comprising a polypeptide sequence that differs from the polypeptide sequence of any of the group consisting of SEQ ID NOS:1-21 and 56-63 by no more than 1, 2, 3, 4, 5, 6, 7, 9, 10, 15, 20, or amino acid residues, wherein the polypeptide variant induces the production of neutralizing antibodies against at least one HIV-1 virus in a subject to whom an effective amount of the variant is administered.

70. The polypeptide of any of claims 1-69, said polypeptide further comprising a signal peptide comprising the sequence of SEQ ID NO:22.

71. The polypeptide of any one of claims 1-70, wherein said polypeptide is linked to at least one additional amino acid sequence, thereby forming a fusion protein.

72. The polypeptide of any one of claims 1-71, comprising a polypeptide purification subsequence.

73. The polypeptide of claim 72, wherein the polypeptide purification subsequence is selected from: an epitope tag, a FLAG tag, a polyhistidine sequence, and a GST fusion.

74. An isolated or recombinant nucleic acid comprising a polynucleotide sequence that encodes the polypeptide of any of claims 1-73 or a complementary polynucleotide sequence thereof.

75. An isolated of recombinant nucleic acid encoding a polypeptide that induces an immune response against an HIV virus, the nucleic acid comprising a polynucleotide sequence having at least 90% sequence identity to at least one sequence selected from the group consisting of SEQ ID NOS:23-50 and 64-79, or a complementary polynucleotide sequence thereof.

76. An isolated or recombinant nucleic acid that induces an immune response against HIV-1 in a subject to whom an effective amount of the nucleic acid is administered, wherein said nucleic acid comprises a polynucleotide sequence having at least 90% sequence identity to an RNA polynucleotide sequence, said RNA polynucleotide sequence comprising a DNA sequence selected from the group of SEQ ID NOS:23-50 and 64-79 in which all of the thymine nucleotide residues in said DNA sequence are replaced with uracil nucleotide residues, or a complementary polynucleotide sequence thereof.

77. The nucleic acid of any of claims 74-76, the nucleic acid further comprising a nucleotide sequence that encodes a signal peptide sequence.

78. The nucleic acid of claim 77, wherein the nucleotide sequence encoding the signal sequence is joined or covalently linked to the polynucleotide sequence that encodes the polypeptide.

79. A composition comprising a polypeptide of any of claims 1-73 and an excipient or carrier.

80. A composition comprising a nucleic acid of any of claims 74-78 and an excipient or carrier.

81. A composition comprising (a) a pharmaceutically acceptable excipient or pharmaceutically acceptable carrier and (b) a polypeptide of any of claims 1-73 and/or a nucleic acid of any of claims 74-78.

82. A vector comprising a nucleic acid of any of claims 74-78 or a nucleic acid that encodes a polypeptide of any of claims 1-73.

83. The vector of claim 82, wherein the vector is a DNA vector.

84. The vector of claim 82, wherein the vector is a RNA vector.

85. The vector of claim 82, wherein the nucleic acid is operably linked to a promoter.

86. The vector of claim 85, wherein the vector is an expression vector.

87. The vector of claim 86, wherein the expression vector comprises the vector shown in FIG. 1.

88. The vector of claim 82, wherein the vector comprises a plasmid, phage, linear expression element, nucleic acid-protein conjugate, virus, pseudovirus, or virus-like particle (VLP).

89. The vector of claim 82, wherein the vector comprises a viral vector, yeast vector, plant vector, or bacterial vector.

90. The vector of claim 89, wherein the viral vector is a replication-deficient or attenuated viral vector.

91. A composition comprising a vector of any of claims 82-90 and an excipient or carrier.

92. A composition comprising a pharmaceutically acceptable excipient or pharmaceutically acceptable carrier and a vector of any of claims 82-90.

93. A virus comprising at least one polypeptide of any of claims 1-73 and/or at least one nucleic acid of any of claims 74-78.

94. A virus-like particle (VLP) comprising at least one polypeptide of any of claims 1-73 and/or at least one nucleic acid of any of claims 74-78.

95. An attenuated or replication deficient virus comprising at least one polypeptide of any of claims 1-73 and/or at least one nucleic acid of any of claims 74-78.

96. A cell comprising at least one polypeptide of any of claims 1-73 and/or at least one nucleic acid of any of claims 74-78.

97. A cell comprising at least one vector of any of claims 82-90.

98. A method of inducing an immune response against at least one HIV-1 virus or HIV-1 pseudovirus in a subject which comprises administering to the subject an effective amount of:
a) at least one polypeptide of any of claims 1-73;
b) at least one nucleic acid of any of claims 74-78;
c) at least one vector comprising a nucleic acid that encodes a polypeptide of any of claims 1-73; or
d) at least one virus or virus-like particle comprising a polypeptide of any of claims 1-73;
or any combination thereof of any of the foregoing, wherein the effective amount induces an immune response against at least one HIV-1 virus or HIV-1 pseudovirus in the subject

99. The method of claim 98, wherein the vector is a DNA vector, RNA vector, plasmid expression vector, phage, linear expression element, nucleic-acid-protein conjugate, viral vector, plant vector, yeast vector, bacterial vector, virus, or virus-like particle.

100. The method of claim 98, wherein the immune response comprises an anti-HIV neutralizing antibody response or an HIV-specific T cell immune response or both.

101. The method of claim 98, wherein the subject is a mammal and the nucleic acid comprises codons optimized for expression in a mammal.

102. A method of inducing an immune response against an HIV-1 virus or HIV-1 pseudovirus in a subject comprising administering to the subject an amount effective to induce said immune response of:
1) at least one nucleic acid having at least 90% sequence identity to a nucleotide sequence selected from the group consisting of SEQ ID NOS:23-50 and 64-79, or a vector comprising the nucleic acid, or a polypeptide encoded by the nucleic acid, wherein the polypeptide encoded by the nucleic acid induces an immune response against at least one HIV-1 virus or pseudovirus;
2) at least one nucleic acid encoding a polypeptide having at least 95% sequence identity to a polypeptide sequence selected from the group consisting of SEQ ID NOS:1-21 and 56-63, or a vector comprising the nucleic acid, or the polypeptide encoded by the nucleic acid, wherein the polypeptide encoded by the nucleic acid induces an immune response against at least one HIV-1 virus or pseudovirus;
3) at least one nucleic acid encoding a polypeptide comprising a fragment of a gp120 variant polypeptide sequence, said gp120 variant polypeptide sequence having at least 95% sequence identity to a sequence selected from the group consisting of SEQ ID NOS:1-7 and 56-63, and the fragment comprises at least those amino acid residues of the gp120 variant polypeptide sequence located at positions corresponding by reference to amino acid residues of regions C2, C3, V4, C4, and V5 of the recombinant HIV-1 gp120-HXB2 envelope protein sequence (SEQ ID NO:54) shown in FIGS. 10A-10F, wherein the amino acid residues of the fragment are numbered by reference to amino acid residues of the recombinant gp120-HXB2 envelope protein, or a vector comprising the nucleic acid, or the polypeptide encoded by the nucleic acid, wherein the polypeptide encoded by the nucleic acid induces an immune response against at least one HIV-1 virus or pseudovirus;
4) at least one nucleic acid encoding a polypeptide comprising a first, a second, a third, a fourth and a fifth subsequence of a gp120 variant sequence, the gp120 variant sequence comprising an amino acid sequence having at least 95% sequence identity to a polypeptide sequence selected from the group consisting of SEQ ID NOS:1-21 and 56-63, wherein: (a) the first subsequence of the gp120 variant sequence comprises a sequence corresponding by reference to amino acid residues 83-127 of the C1 region of the recombinant HXB2 gp120 envelope protein (SEQ ID NO:54) shown in FIGS. 10A-10F, wherein the C-terminus of the first subsequence is covalently linked by a peptide bond to the N-terminus of a first linker peptide; (b) the second subsequence of the gp120 variant sequence corresponds by reference to the C2 region of the recombinant HXB2 gp120 envelope protein (SEQ ID NO:54) shown in FIGS. 10A-10F, wherein the N-terminus of the second subsequence is covalently linked by a peptide bond to the C-terminus of the first linker peptide, and the C-terminus of the second subsequence is covalently linked by a peptide bond to the N-terminus of a second linker peptide or a gp120 V3 region sequence; (c) the third subsequence of the gp120 variant sequence corresponds by reference to the C3 region of the recombinant HXB2 gp120 envelope protein (SEQ ID NO:54) shown in FIGS. 10A-10F, and the N-terminus of the third subsequence is covalently linked by a peptide bond to the C-terminus of the second linker polypeptide or the gp120 V3 region sequence, and the C-terminus of the third subsequence is covalently linked by a peptide bond to the N-terminus of a gp120 V4 region sequence; (d) the fourth subsequence of the gp120 variant sequence corresponds by reference to the C4 region of the recombinant HXB2 gp120 envelope protein (SEQ ID NO:54) shown in FIGS. 10A-10F, and the N-terminus of the fourth subsequence is covalently linked by a peptide bond to the C-terminus of the gp120 V4 region sequence, and the C-terminus of the fourth subsequence is covalently linked by a peptide bond to the N-terminus of a gp120 V5 region sequence; and (e) the fifth subsequence of the gp120 variant sequence comprises a sequence corresponding by reference to amino acid residues 472-492 of the C5 region of the recombinant HXB2 gp120 envelope protein (SEQ ID NO:54) shown in FIGS. 10A-10F, and the N-terminus of the fifth subsequence is covalently linked by a peptide bond to the C-terminus of the V5 region sequence; wherein the gp120 V3 region sequence, the gp120 V4 region sequence and the gp120 V5 region sequence correspond by reference to the V3 region, the V4 region, and the V5 region, respectively, of the recombinant HXB2 gp120 envelope protein (SEQ ID NO:54) shown in FIGS. 10A-10F, and one or more of the gp120 V3 region sequence, the gp120 V4 region sequence and the gp120 V5 region sequence is not a subsequence of the selected gp120 variant sequence, or, a vector comprising the nucleic acid, or the polypeptide encoded by the nucleic acid, wherein the polypeptide encoded by the nucleic acid induces an immune response against at least one HIV-1 virus or pseudovirus;
5) at least one virus or virus-like particle (VLP) comprising the nucleic acid of (1), (2), (3), or (4) or the polypeptide encoded by the nucleic acid of (1), (2), (3), or (4) wherein the polypeptide encoded by the nucleic acid induces an immune response against at least one HIV-1 virus or pseudovirus;

103. The method of claim 102, wherein the nucleic acid is selected from the group consisting of SEQ ID NOS:23-50 and 64-79 and the polypeptide is selected from the group consisting of SEQ ID NOS:1-21 and 56-63.

104. The method of claim 102, wherein the immune response comprises an anti-HIV neutralizing antibody response or an HIV-specific T cell immune response or both.

105. The method of claim 104, wherein the immune response comprises production of neutralizing antibodies against at least two HIV-1 viruses.

106. The method of claim 105, wherein the at least two HIV-1 viruses comprise the same HIV-1 virus subtype.

107. The method of claim 105, wherein each of the at least two HIV-1 viruses comprises HIV-1 virus subtype B.

108. The method of claim 105, wherein the at least two HIV-1 viruses comprise different HIV-1 subtypes.

109. A method of reducing or inhibiting HIV-1 transmission or infection of cells by HIV-1 in a subject, the method comprising administering to the subject an effective amount of:
a) at least one polypeptide of any of claims 1-73;
b) at least one nucleic acid of any of claims 74-78;
c) at least one vector comprising a nucleic acid that encodes a polypeptide of any of claims 1-73; or
d) at least one virus or virus-like particle comprising a polypeptide of any of claims 1-73; or any combination thereof of any of the foregoing,
wherein the effective amount reduces or inhibits HIV-1 cellular infection or transmission in the subject.

110. A method of inducing an immune response against HIV-1 in a subject, comprising administering to the subject an amount of a nucleic acid of any of claims 74-78 effective to induce the immune response, wherein the nucleic acid is operably linked to a promoter sequence that controls the expression of said nucleic acid, and the polynucleotide is present in an amount sufficient such that uptake of the polynucleotide into one or more cells of the subject and expression of the nucleic acid occurs to induce the immune response.

111. The method of claim 110, further comprising administering to the subject an amount of a polypeptide of any of claims 1-73 effective to induce or enhance the immune response.

112. An isolated antibody which specifically binds a polypeptide comprising a polypeptide sequence selected from the group consisting of SEQ ID NOS:1-21 and 56-63.

113. The isolated antibody of claim 112, wherein the antibody is a monoclonal antibody.

114. An isolated antiserum which specifically binds a polypeptide comprising a polypeptide sequence selected from the group consisting of SEQ ID NOS:1-21 and 56-63.

115. An isolated antibody or antiserum produced by administering a polypeptide of any of claims 1-73 to a subject.

116. An immortalized cell line that produces the antibody of claim 112.

117. A method of producing a polypeptide comprising culturing the cell of claim 97 in an appropriate medium and recovering the polypeptide expressed by the cell.

118. A method of producing a polypeptide, comprising:
(a) introducing into a population of cells a nucleic acid of any of claims 74-78, wherein the nucleic acid is operatively linked to a regulatory sequence effective to produce the polypeptide encoded by the nucleic acid;
(b) culturing the cells in a culture medium to produce the polypeptide; and
(c) isolating the polypeptide from the cells or culture medium.

119. A method of producing a polypeptide, the method comprising:
(a) introducing into a population of cells an expression vector comprising the nucleic acid of any of claims 74-78;
(b) administering the expression vector into a subject; and
(c) isolating the polypeptide from the subject or from a byproduct of the subject.

120. A method of generating a cytotoxic T cell response in a subject, the method comprising administering to the subject an effective amount of a nucleic acid comprising a nucleotide sequence encoding at least one polypeptide of any of claims 1-73, wherein the nucleotide sequence is under the control of a promoter that is capable of expressing the polypeptide in the subject.

121. The method of claim 120, wherein the vector is a plasmid vector, viral vector, bacterial vector, yeast vector, or plant vector.

122. A method of generating a cytotoxic T cell response in a subject, the method comprising administering to the subject at least one polypeptide of any of claims 1-73.

123. Use of a polypeptide of any of claims 1-73 or a nucleic acid of any of claims 74-78 or a vector of any of claims 82-90 for the manufacture of a medicament for inducing an immune response against at least one HIV virus.

124. Use according to claim 123, wherein the immune response is a neutralizing antibody response or a T cell response against at least one HIV-1 virus.

125. Use of a polypeptide of any of claims 1-73 or a nucleic acid of any of claims 74-78 or a vector of any of claims 82-90 for the preparation of a medicament for inhibiting or preventing infection of cells in a subject by at least one HIV-1 virus.

126. A pseudovirus comprising at least one polypeptide of any of claims 1-73 and/or at least one nucleic acid of any of claims 74-78.

127. Use of a pseudovirus of claim 126 for the manufacture of a medicament for inducing an immune response against at least one HIV virus.

128. Use of a virus-like particle (VLP) of claim 94 for the manufacture of a medicament for inducing an immune response against at least one HIV virus.
